(19) United States
(12) Patent Application Publication
(10) Pub. No.: US 2024/0368121 A1
(43) Pub. Date: Nov. 7, 2024

(54) ESTROGEN RECEPTOR DEGRADERS, PHARMACEUTICAL COMPOSITIONS THEREOF, AND THEIR THERAPEUTIC APPLICATIONS

(71) Applicant: BioTheryX, Inc., San Diego, CA (US)

(72) Inventors: Kyle W.H. Chan, San Diego, CA (US); Aparajita Hoskote Chourasia, San Diego, CA (US); Paul E. Erdman, San Diego, CA (US); Leah M. Fung, San Diego, CA (US); Imelda Lam, San Diego, CA (US); Patrick Papa, Carlsbad, CA (US); Angela Schoolmeesters, San Diego, CA (US); Eduardo Torres, San Diego, CA (US)

(21) Appl. No.: 18/684,902

(22) PCT Filed: Aug. 16, 2022
(86) PCT No.: PCT/US2022/075043

§ 371 Date: Feb. 20, 2024

Related U.S. Application Data
(60) Provisional application No. 63/235,624, filed on Aug. 20, 2021.

(51) Int. Cl. C07D 401/14; A61K 31/451; A61K 31/454; A61K 31/4545; A61K 31/513; C07D 211/88; C07D 239/22; C07D 401/04; C07D 495/04

(52) U.S. Cl. CPC C07D 401/14; A61K 31/451; A61K 31/454; A61K 31/4545; A61K 31/513; C07D 211/88; C07D 239/22; C07D 401/04; C07D 495/04

(57) ABSTRACT

Provided herein are estrogen receptor degraders, for example, a compound of Formula (I), R^{T}-L-R^{E}; and pharmaceutical compositions thereof. Also provided herein are methods of their use for treating, preventing, or ameliorating one or more symptoms r of an estrogen receptor-mediated disorder, disease, or condition.

### CROSS REFERENCE TO RELATED APPLICATION

This application claims the benefit of the priority of U.S. Provisional Application No. 63/235,624, filed Aug. 20, 2021; the disclosure of which is incorporated herein by reference in its entirety.

### FIELD

Provided herein are estrogen receptor degraders and pharmaceutical compositions thereof. Also provided herein are methods of their use for treating, preventing, or ameliorating one or more symptoms of an estrogen receptor-mediated disorder, disease, or condition.

### BACKGROUND

Estrogen is a steroid hormone that plays a critical role in reproductive development, bone homeostasis, cardiovascular remodeling, and brain functions. Hua et al., Exp. Hematol. Oncol. 2018, 7, 24. Estrogen also plays a vital role in cancer development and progression. Folkerd and Dowsett, J. Clin. Oncol. 2010, 28, 4038-44; Hua et al., Exper. Hemat. Oncol. 2018, 7, 24; Piggoot et al., Clin. Cancer Res. 2018, 24, 2452-63. The biological effects of estrogen are mostly mediated by its binding and activation of an estrogen receptor (ER). Hewitt and Korach, Endocr. Rev. 2018, 39, 664-75. In mammals, there are two ERs, ERα and Erβ, both members of the nuclear receptor superfamily of hormone-inducible transcription factors. Aranda and Pascual, Physiol. Rev. 2001, 81, 1269-304; Hewitt and Korach, Endocr. Rev. 2018, 39, 664-75.

Breast cancer is one of the most common cancer in women worldwide. Harbeck et al., Nat. Rev. Dis. Primers 2019, 5, 66. According to the World Health Organization, in 2020, there were 2.3 million women diagnosed with breast cancer and 685 000 deaths globally. Approximately 75% of breast cancer are ERα-positive at diagnosis. Hua et al., Exp. Hematol. Oncol. 2018, 7, 24; Harbeck et al., Nat. Rev. Dis. Primers 2019, 5, 66. Endocrine therapy is the first-line treatment for ERα-positive breast cancer. Shagufta et al., RSCMed. Chem. 2020, 11, 438-54. Unfortunately, up to 50% of ERα-positive breast cancer patients following initial responsiveness become resistant to endocrine therapy within 5 years. Piggoot et al., Clin. Cancer Res. 2018, 24, 2452-63; Szostakowska et al., Breast Cancer Res. Treat. 2019, 173, 489-97; Lu and Liu, J. Med. Chem. 2020, 63, 15094-114. Such acquired resistance is associated with poor prognosis and remains an area of an unmet clinical need. Piggoot et al., Clin. Cancer Res. 2018, 24, 2452-63; Harbeck et al., Nat. Rev. Disease Primers 2019, 5. 66; Shagufta et al., RSC Med. Chem. 2020, 11, 438-54. Therefore, there is a need for an effective therapy for breast cancer, especially recurrent ERα-positive breast cancer.

### SUMMARY OF THE DISCLOSURE

Provided herein is a compound of Formula (I):

R^{T}-L-R^{E} (I)

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
L is a linker;
R^{E} is an E3 ubiquitin ligase binding moiety; and
R^{T} is a moiety having the structure of any one of Formulae (i) to (xv):

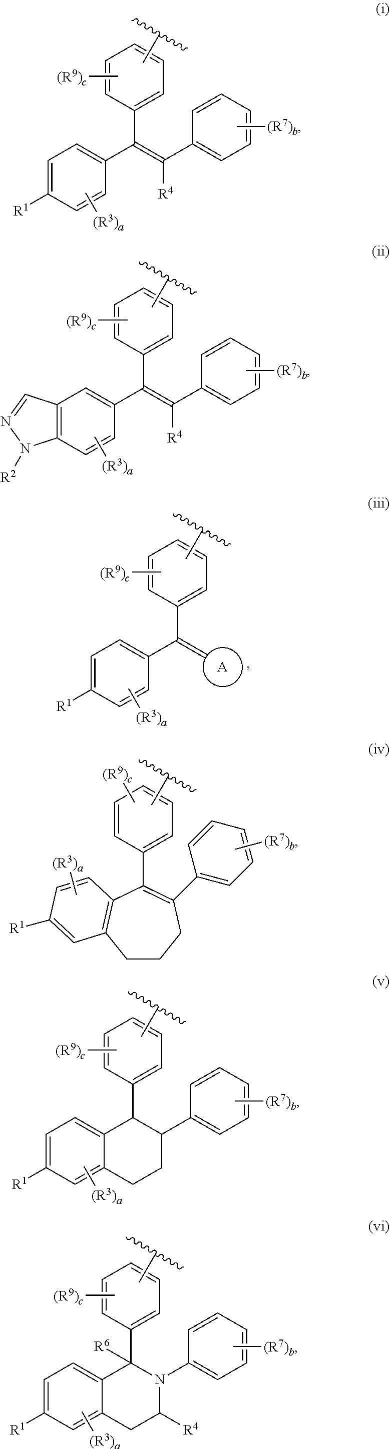

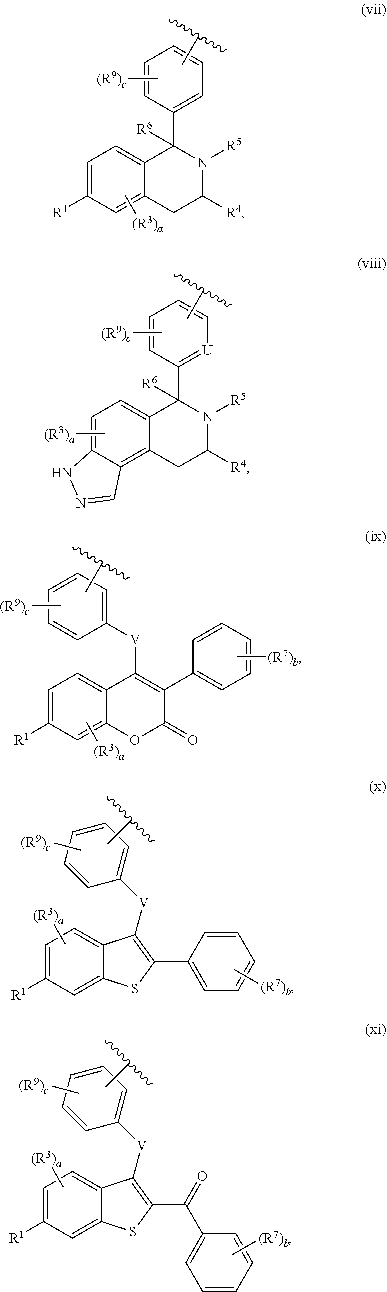

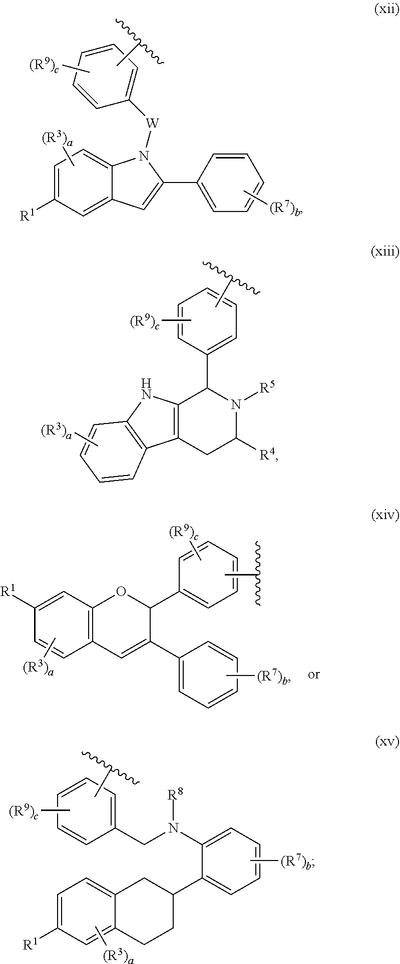

wherein:
ring A is C₃₋₁₀ cycloalkylene or heterocyclylene;
U is —CH═ or —N═;
each V is independently —O—, —S—, —N(R^{1a})—, —C(O)—, or C₁₋₆ alkylene;
W is —O—, —N(R^{1a})—, —C(O)—, or C₁₋₆ alkylene;
each R¹ is independently hydrogen, deuterium, halo, —C(O)OR^{1a}, —OR^{1a}, or —NR^{1b}R^{1c};
each R², R⁵, and R⁸ is independently (i) hydrogen; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c};
each R³, R⁷, and R⁹ is independently (i) hydrogen, deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each R⁴ and R⁶ is independently hydrogen, deuterium, C₁₋₆ alkyl, or C₁₋₆ heteroalkyl;
each a and c is independently an integer of 0, 1, 2, 3, or 4;
each b is independently an integer of 0, 1, 2, 3, 4, or 5; and
each R^{1a}, R^{1b}, R^{1c}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
wherein each alkyl, alkylene, heteroalkyl, alkenyl, alkynyl, cycloalkyl, cycloalkylene, aryl, aralkyl, heteroaryl, heterocyclyl, and heterocyclylene is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{b})OR^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR)NR^{f}R^{g}, —OC(S)R^{e}, —OC(S)OR^{e}, —OC(S)NR^{f}R^{g}, —OP(O)(OR^{f})OR⁹, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

Also provided herein is a pharmaceutical composition comprising a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; and a pharmaceutically acceptable excipient.

Additionally, provided herein is a method of treating, preventing, or ameliorating one or more symptoms of a disorder, disease, or condition mediated by an estrogen receptor in a subject, comprising administering to the subject in need thereof a therapeutically effective amount of a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

Furthermore, provided herein is a method of treating, preventing, or ameliorating one or more symptoms of a proliferative disease in a subject, comprising administering to the subject in need thereof a therapeutically effective amount of a compound of Formula (I), or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof.

Provided herein is a method of inhibiting the growth of a cell, comprising contacting the cell with an effective amount of a compound of Formula (I), or an enantiomer, a mixture of enantiomers, a mixture of two or more diastereomers, a tautomer, a mixture of two or more tautomers, or an isotopic variant thereof; or a pharmaceutically acceptable salt, solvate, hydrate, or prodrug thereof.

Provided herein is a method of inducing degradation of an estrogen receptor, comprising contacting the estrogen receptor with an effective amount of a compound of Formula (I), or an enantiomer, a mixture of enantiomers, a mixture of two or more diastereomers, a tautomer, a mixture of two or more tautomers, or an isotopic variant thereof, or a pharmaceutically acceptable salt, solvate, hydrate, or prodrug thereof.

### DETAILED DESCRIPTION

To facilitate understanding of the disclosure set forth herein, a number of terms are defined below.

Generally, the nomenclature used herein and the laboratory procedures in organic chemistry, medicinal chemistry, biochemistry, biology, and pharmacology described herein are those well-known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject. In one embodiment, the subject is a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “alleviate” and “alleviating” refer to easing or reducing one or more symptoms (e.g., pain) of a disorder, disease, or condition. The terms can also refer to reducing adverse effects associated with an active ingredient. Sometimes, the beneficial effects that a subject derives from a prophylactic or therapeutic agent do not result in a cure of the disorder, disease, or condition.

The term “contacting” or “contact” is meant to refer to bringing together of a therapeutic agent and a biological molecule (e.g., a protein, enzyme, RNA, or DNA), cell, or tissue such that a physiological and/or chemical effect takes place as a result of such contact. Contacting can take place in vitro, ex vivo, or in vivo. In one embodiment, a therapeutic agent is contacted with a biological molecule in vitro to determine the effect of the therapeutic agent on the biological molecule. In another embodiment, a therapeutic agent is contacted with a cell in cell culture (in vitro) to determine the effect of the therapeutic agent on the cell. In yet another embodiment, the contacting of a therapeutic agent with a biological molecule, cell, or tissue includes the administration of a therapeutic agent to a subject having the biological molecule, cell, or tissue to be contacted.

The term “therapeutically effective amount” or “effective amount” is meant to include the amount of a compound that, when administered, is sufficient to prevent development of, or alleviate to some extent, one or more of the symptoms of the disorder, disease, or condition being treated. The term “therapeutically effective amount” or “effective amount” also refers to the amount of a compound that is sufficient to elicit a biological or medical response of a biological molecule (e.g., a protein, enzyme, RNA, or DNA), cell, tissue, system, animal, or human, which is being sought by a researcher, veterinarian, medical doctor, or clinician.

The term “IC₅₀” or “EC₅₀” refers to an amount, concentration, or dosage of a compound that is required for 50% inhibition of a maximal response in an assay that measures such a response.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of a subject (e.g., a human) without excessive toxicity, irritation, allergic response, immunogenicity, or other problems or complications, and commensurate with a reasonable benefit/risk ratio. See, e.g., Remington: The Science and Practice of Pharmacy, 23rd ed.; Adejare Ed.; Academic Press, 2020; Handbook of Pharmaceutical Excipients, 9th ed.; Sheskey et al., Eds.; Pharmaceutical Press, 2020; Handbook of Pharmaceutical Additives, 3rd ed.; Ash and Ash Eds.; Synapse Information Resources, 2007; Pharmaceutical Preformulation and Formulation, 1st ed.; Gibson Ed.; CRC Press, 2015.

The term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 25%, 20%, 15%, 10%, 9%, 8%, 7%, 6%, 5%, 4%, 3%, 2%, 1%, 0.5%, or 0.05% of a given value or range.

The term “alkyl” refers to a linear or branched saturated monovalent hydrocarbon radical, wherein the alkyl is optionally substituted with one or more substituents Q as described herein. For example, C₁₋₆ alkyl refers to a linear saturated monovalent hydrocarbon radical of 1 to 6 carbon atoms or a branched saturated monovalent hydrocarbon radical of 3 to 6 carbon atoms. In certain embodiments, the alkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms, e.g., n-propyl and isopropyl), butyl (including all isomeric forms, e.g., n-butyl, isobutyl, sec-butyl, and t-butyl), pentyl (including all isomeric forms, e.g., n-pentyl, isopentyl, sec-pentyl, neopentyl, and tert-pentyl), and hexyl (including all isomeric forms, e.g., n-hexyl, isohexyl, and sec-hexyl).

The term “heteroalkyl” refers to a linear or branched saturated monovalent hydrocarbon radical that contains one or more heteroatoms on its main chain, each independently selected from O, S, and N. The heteroalkyl is optionally substituted with one or more substituents Q as described herein. For example, C₁₋₆ heteroalkyl refers to a linear saturated monovalent hydrocarbon radical of 1 to 6 carbon atoms or a branched saturated monovalent hydrocarbon radical of 3 to 6 carbon atoms. In certain embodiments, the heteroalkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ heteroalkyl groups are also referred as “lower heteroalkyl.” Examples of heteroalkyl groups include, but are not limited to, —OCH₃, —OCH₂CH₃, —CH₂OCH₃, —NHCH₃, —ONHCH₃, —NHOCH₃, —SCH₃, —CH₂NHCH₂CH₃, and —NHCH₂CH₂CH₃. Examples of substituted heteroalkyl groups include, but are not limited to, —CH₂NHC(O)CH₃ and —NHC(O)CH₂CH₃.

The terms “alkylene” and “alkanediyl” are used interchangeably herein in reference to a linear or branched saturated divalent hydrocarbon radical, wherein the alkanediyl is optionally be substituted with one or more substituents Q as described herein. For example, C₁₋₆ alkanediyl refers to a linear saturated divalent hydrocarbon radical of 1 to 6 carbon atoms or a branched saturated divalent hydrocarbon radical of 3 to 6 carbon atoms. In certain embodiments, the alkanediyl is a linear saturated divalent hydrocarbon radical that has 1 to 30 (C₁₋₃₀), 1 to 20 (C₁₋₂₀), 1 to 15 (C₁₋₁₅), 1 to 10 (C₁₋₁₀), or 1 to 6 (C₁₋₆) carbon atoms, or branched saturated divalent hydrocarbon radical of 3 to 30 (C₃₋₃₀), 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkanediyl groups are also referred as “lower alkanediyl.” Examples of alkanediyl groups include, but are not limited to, methanediyl, ethanediyl (including all isomeric forms, e.g., ethane-1,1-diyl and ethane-1,2-diyl), propanediyl (including all isomeric forms, e.g., propane-1,1-diyl, propane-1,2-diyl, and propane-1,3-diyl), butanediyl (including all isomeric forms, e.g., butane-1,1-diyl, butane-1,2-diyl, butane-1,3-diyl, and butane-1,4-diyl), pentanediyl (including all isomeric forms, e.g., pentane-1,1-diyl, pentane-1,2-diyl, pentane-1,3-diyl, and pentane-1,5-diyl), and hexanediyl (including all isomeric forms, e.g., hexane-1,1-diyl, hexane-1,2-diyl, hexane-1,3-diyl, and hexane-1,6-diyl). Examples of substituted alkanediyl groups include, but are not limited to, —C(O)CH₂—, —C(O)(CH₂)₂—, —C(O)(CH₂)₃—, —C(O)(CH₂)₄—, —C(O)(CH₂)₅—, —C(O)(CH₂)₆—, —C(O)(CH₂)₇—, —C(O)(CH₂)₈—, —C(O)(CH₂)₉—, —C(O)(CH₂)₁₀—, —C(O)CH₂C(O)—, —C(O)(CH₂)₂C(O)—, —C(O)(CH₂)₃C(O)—, —C(O)(CH₂)₄C(O)—, or —C(O)(CH₂)₅C(O)—.

The terms “heteroalkylene” and “heteroalkanediyl” are used interchangeably herein in reference to a linear or branched saturated divalent hydrocarbon radical that contains one or more heteroatoms in its main chain, each independently selected from O, S, and N. The heteroalkylene is optionally substituted with one or more substituents Q as described herein. For example, C₁₋₆ heteroalkylene refers to a linear saturated divalent hydrocarbon radical of 1 to 6 carbon atoms or a branched saturated divalent hydrocarbon radical of 3 to 6 carbon atoms. In certain embodiments, the heteroalkylene is a linear saturated divalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ heteroalkylene groups are also referred as “lower heteroalkylene.” Examples of heteroalkylene groups include, but are not limited to, —CH₂O—, —(CH₂)₂O—, —(CH₂)₃O—, —(CH₂)₄O—, —(CH₂)₅O—, —(CH₂)₆O—, —(CH₂)₇O—, —(CH₂)₈O—, —(CH₂)₉O—, —(CH₂)₁₀O—, —CH₂OCH₂—, —CH₂CH₂O—, —(CH₂CH₂O)₂—, —(CH₂CH₂O)₃—, —(CH₂CH₂O)₄—, —(CH₂CH₂O)₅—, —CH₂NH—, —CH₂NHCH₂—, —CH₂CH₂NH—, —CH₂S—, —CH₂SCH₂—, and —CH₂CH₂S—. Examples of substituted heteroalkylene groups include, but are not limited to, —C(O)CH₂O—, —C(O)(CH₂)₂O—, —C(O)(CH₂)₃O—, —C(O)(CH₂)₄O—, —C(O)(CH₂)₅O—, —C(O)(CH₂)₆O—, —C(O)(CH₂)₇O—, —C(O)(CH₂)₈O—, —C(O)(CH₂)₉O—, —C(O)(CH₂)₁₀O—, —C(O)CH₂OCH₂CH₂O—, —C(O)CH₂O(CH₂CH₂O)₂—, —C(O)CH₂O(CH₂CH₂O)₃—, —C(O)CH₂O(CH₂CH₂O)₄, —C(O)CH₂O(CH₂CH₂O)₅—, —CH₂NHC(O)CH₂—, or —CH₂CH₂C(O)NH—.

The term “alkenyl” refers to a linear or branched monovalent hydrocarbon radical, which contains one or more, in one embodiment, one, two, three, or four, in another embodiment, one, carbon-carbon double bond(s). The alkenyl is optionally substituted with one or more substituents Q as described herein. The term “alkenyl” embraces radicals having a “cis” or “trans” configuration or a mixture thereof, or alternatively, a “Z” or “E” configuration or a mixture thereof, as appreciated by those of ordinary skill in the art. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenyl groups include, but are not limited to, ethenyl, propenyl (including all isomeric forms, e.g., propen-1-yl, propen-2-yl, and allyl), and butenyl (including all isomeric forms, e.g., buten-1-yl, buten-2-yl, buten-3-yl, and 2-buten-1-yl).

The terms “alkenylene” and “alkenediyl” are used interchangeably herein in reference to a linear or branched divalent hydrocarbon radical, which contains one or more, in one embodiment, one, two, three, or four, in another embodiment, one, carbon-carbon double bond(s). The alkenediyl is optionally substituted with one or more substituents Q as described herein. The term “alkenediyl” embraces radicals having a “cis” or “trans” configuration or a mixture thereof, or alternatively, a “Z” or “E” configuration or a mixture thereof, as appreciated by those of ordinary skill in the art. For example, C₂₋₆ alkenediyl refers to a linear unsaturated divalent hydrocarbon radical of 2 to 6 carbon atoms or a branched unsaturated divalent hydrocarbon radical of 3 to 6 carbon atoms. In certain embodiments, the alkenediyl is a linear divalent hydrocarbon radical of 2 to 30 (C₂₋₃₀), 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 3 to 30 (C₃₋₃₀), 3 to 20 (C₃₋₂₀), 3 to 15 (C₃₋₁₅), 3 to 10 (C₃₋₁₀), or 3 to 6 (C₃₋₆) carbon atoms. Examples of alkenediyl groups include, but are not limited to, ethenediyl (including all isomeric forms, e.g., ethene-1,1-diyl and ethene-1,2-diyl), propenediyl (including all isomeric forms, e.g., 1-propene-1,1-diyl, 1-propene-1,2-diyl, and 1-propene-1,3-diyl), butenediyl (including all isomeric forms, e.g., 1-butene-1,1-diyl, 1-butene-1,2-diyl, and 1-butene-1,4-diyl), pentenediyl (including all isomeric forms, e.g., 1-pentene-1,1-diyl, 1-pentene-1,2-diyl, and 1-pentene-1,5-diyl), and hexenediyl (including all isomeric forms, e.g., 1-hexene-1,1-diyl, 1-hexene-1,2-diyl, 1-hexene-1,3-diyl, 1-hexene-1,4-diyl, 1-hexene-1,5-diyl, and 1-hexene-1,6-diyl).

The terms “heteroalkenylene” and “heteroalkenediyl” are used interchangeably herein in reference to a linear or branched divalent hydrocarbon radical, which contains one or more, in one embodiment, one, two, three, or four, in another embodiment, one, carbon-carbon double bond(s), and which contains one or more heteroatoms each independently selected from O, S, and N in the hydrocarbon chain. The heteroalkenylene is optionally substituted with one or more substituents Q as described herein. The term “heteroalkenylene” embraces radicals having a “cis” or “trans” configuration or a mixture thereof, or alternatively, a “Z” or “E” configuration or a mixture thereof, as appreciated by those of ordinary skill in the art. For example, C₂₋₆ heteroalkenylene refers to a linear unsaturated divalent hydrocarbon radical of 2 to 6 carbon atoms or a branched unsaturated divalent hydrocarbon radical of 3 to 6 carbon atoms. In certain embodiments, the heteroalkenylene is a linear divalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of heteroalkenylene groups include, but are not limited to, —CH═CHO—, —CH═CHOCH₂—, —CH═CHCH₂O—, —CH═CHS—, —CH═CHSCH₂—, —CH═CHCH₂S—, or —CH═CHCH₂NH—.

The term “alkynyl” refers to a linear or branched monovalent hydrocarbon radical, which contains one or more, in one embodiment, one, two, three, or four, in another embodiment, one, carbon-carbon triple bond(s). An alkynyl group does not contain a carbon-carbon double bond. The alkynyl is optionally substituted with one or more substituents Q as described herein. For example, C₂₋₆ alkynyl refers to a linear unsaturated monovalent hydrocarbon radical of 2 to 6 carbon atoms or a branched unsaturated monovalent hydrocarbon radical of 4 to 6 carbon atoms. In certain embodiments, the alkynyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 4 to 20 (C₄₋₂₀), 4 to 15 (C₄₋₁₅), 4 to 10 (C₄-10), or 4 to 6 (C₄₋₆) carbon atoms. Examples of alkynyl groups include, but are not limited to, ethynyl (—C≡CH), propynyl (including all isomeric forms, e.g., 1-propynyl (—C≡CCH₃) and propargyl (—CH₂C≡CH)), butynyl (including all isomeric forms, e.g., 1-butyn-1-yl and 2-butyn-1-yl), pentynyl (including all isomeric forms, e.g., 1-pentyn-1-yl and 1-methyl-2-butyn-1-yl), and hexynyl (including all isomeric forms, e.g., 1-hexyn-1-yl and 2-hexyn-1-yl).

The terms “alkynylene” and “alkynediyl” are used interchangeably herein in reference to a linear or branched divalent hydrocarbon radical, which contains one or more, in one embodiment, one, two, three, or four, in another embodiment, one, carbon-carbon triple bond(s). An alkynylene group does not contain a carbon-carbon double bond. The alkynediyl is optionally substituted with one or more substituents Q as described herein. For example, C₂₋₆ alkynediyl refers to a linear unsaturated divalent hydrocarbon radical of 2 to 6 carbon atoms or a branched unsaturated divalent hydrocarbon radical of 4 to 6 carbon atoms. In certain embodiments, the alkynediyl is a linear divalent hydrocarbon radical of 2 to 30 (C₂₋₃₀), 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 4 to 30 (C₄₋₃₀), 4 to 20 (C₄₋₂₀), 4 to 15 (C₄₋₁₅), 4 to 10 (C₄₋₁₀), or 4 to 6 (C₄₋₆) carbon atoms. Examples of alkynediyl groups include, but are not limited to, ethynediyl, propynediyl (including all isomeric forms, e.g., 1-propyne-1,3-diyl and 1-propyne-3,3-diyl), butynediyl (including all isomeric forms, e.g., 1-butyne-1,3-diyl, 1-butyne-1,4-diyl, and 2-butyne-1,1-diyl), pentynediyl (including all isomeric forms, e.g., 1-pentyne-1,3-diyl, 1-pentyne-1,4-diyl, and 2-pentyne-1,1-diyl), and hexynediyl (including all isomeric forms, e.g., 1-hexyne-1,3-diyl, 1-hexyne-1,4-diyl, and 2-hexyne-1,1-diyl).

The terms “heteroalkynylene” and “heteroalkynediyl” are used interchangeably herein in reference to a linear or branched divalent hydrocarbon radical, which contains one or more, in one embodiment, one, two, three, or four, in another embodiment, one, carbon-carbon triple bond(s), and which contains one or more heteroatoms in its main chain, each independently selected from O, S, and N. A heteroalkynylene group does not contain a carbon-carbon double bond. The heteroalkynylene is optionally substituted with one or more substituents Q as described herein. For example, C₂₋₆ heteroalkynylene refers to a linear unsaturated divalent hydrocarbon radical of 2 to 6 carbon atoms or a branched unsaturated divalent hydrocarbon radical of 4 to 6 carbon atoms. In certain embodiments, the heteroalkynylene is a linear divalent hydrocarbon radical of 2 to 30 (C₂₋₃₀), 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 4 to 30 (C₄₋₃₀), 4 to 20 (C₄₋₂₀), 4 to 15 (C₄₋₁₅), 4 to 10 (C₄₋₁₀), or 4 to 6 (C₄₋₆) carbon atoms. Examples of heteroalkynylene groups include, but are not limited to, —C≡CCH₂O—, —C≡CCH₂S—, or —C≡CCH₂NH—.

The term “cycloalkyl” refers to a cyclic monovalent hydrocarbon radical, which is optionally substituted with one or more substituents Q as described herein. In one embodiment, the cycloalkyl is a saturated or unsaturated but non-aromatic, and/or bridged or non-bridged, and/or fused bicyclic group. In certain embodiments, the cycloalkyl has from 3 to 20 (C₃₋₂₀), from 3 to 15 (C₃₋₁₅), from 3 to 10 (C₃₋₁₀), or from 3 to 7 (C₃₋₇) carbon atoms. In one embodiment, the cycloalkyl is monocyclic. In another embodiment, the cycloalkyl is bicyclic. In yet another embodiment, the cycloalkyl is tricyclic. In still another embodiment, the cycloalkyl is polycyclic. Examples of cycloalkyl groups include, but are not limited to, cyclopropyl, cyclobutyl, cyclopentyl, cyclopentenyl, cyclohexyl, cyclohexenyl, cyclohexadienyl, cycloheptyl, cycloheptenyl, bicyclo[1.1.1]pentyl, bicyclo[2.1.1]hexyl, bicyclo[2.2.1]heptyl, bicyclo[2.2.2]octyl, decalinyl, and adamantyl.

The terms “cycloalkylene” and “cycloalkanediyl” are used interchangeably herein in reference to a cyclic divalent hydrocarbon radical, which may be optionally substituted with one or more substituents Q as described herein. In one embodiment, cycloalkanediyl groups may be saturated or unsaturated but non-aromatic, and/or bridged, and/or non-bridged, and/or fused bicyclic groups. In certain embodiments, the cycloalkanediyl has from 3 to 30 (C₃₋₃₀), 3 to 20 (C₃₋₂₀), from 3 to 15 (C₃₋₁₅), from 3 to 10 (C₃₋₁₀), or from 3 to 7 (C₃₋₇) carbon atoms. Examples of cycloalkanediyl groups include, but are not limited to, cyclopropanediyl (including all isomeric forms, e.g., cyclopropane-1,1-diyl and cyclopropane-1,2-diyl), cyclobutanediyl (including all isomeric forms, e.g., cyclobutane-1,1-diyl, cyclobutane-1,2-diyl, and cyclobutane-1,3-diyl), cyclopentanediyl (including all isomeric forms, e.g., cyclopentane-1,1-diyl, cyclopentane-1,2-diyl, and cyclopentane-1,3-diyl), cyclohexanediyl (including all isomeric forms, e.g., cyclohexane-1,1-diyl, cyclohexane-1,2-diyl, cyclohexane-1,3-diyl, and cyclohex-1,4-diyl), cycloheptanediyl (including all isomeric forms, e.g., cycloheptane-1,1-diyl, cycloheptane-1,2-diyl, cycloheptane-1,3-diyl, and cycloheptane-1,4-diyl), decalinediyl (including all isomeric forms, e.g., decaline-1,1-diyl, decaline-1,2-diyl, and decaline-1,8-diyl), and adamantdiyl (including all isomeric forms, e.g., adamant-1,2-diyl, adamant-1,3-diyl, and adamant-1,8-diyl).

The term “aryl” refers to a monovalent monocyclic aromatic hydrocarbon radical and/or monovalent polycyclic aromatic hydrocarbon radical that contain at least one aromatic carbon ring. In certain embodiments, the aryl has from 6 to 20 (C₆₋₂₀), from 6 to 15 (C₆₋₁₅), or from 6 to 10 (C₆₋₁₀) ring carbon atoms. Examples of aryl groups include, but are not limited to, phenyl, naphthyl, fluorenyl, azulenyl, anthryl, phenanthryl, pyrenyl, biphenyl, and terphenyl. The aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl). In one embodiment, the aryl is monocyclic. In another embodiment, the aryl is bicyclic. In yet another embodiment, the aryl is tricyclic. In still another embodiment, the aryl is polycyclic. In certain embodiments, the aryl is optionally substituted with one or more substituents Q as described herein.

The terms “arylene” and “arenediyl” are used interchangeably herein in reference to a divalent monocyclic aromatic hydrocarbon radical or divalent polycyclic aromatic hydrocarbon radical that contains at least one aromatic hydrocarbon ring. In certain embodiments, the arylene has from 6 to 20 (C₆₋₂₀), from 6 to 15 (C₆₋₁₅), or from 6 to 10 (C₆₋₁₀) ring atoms. Examples of arylene groups include, but are not limited to, phenylene (including all isomeric forms, e.g., phen-1,2-diyl, phen-1,3-diyl, and phen-1,4-diyl), naphthylene (including all isomeric forms, e.g., naphth-1,2-diyl, naphth-1,3-diyl, and naphth-1,8-diyl), fluorenylene (including all isomeric forms, e.g., fluoren-1,2-diyl, fluoren-1,3-diyl, and fluoren-1,8-diyl), azulenylene (including all isomeric forms, e.g., azulen-1,2-diyl, azulen-1,3-diyl, and azulen-1,8-diyl), anthrylene (including all isomeric forms, e.g., anthr-1,2-diyl, anthr-1,3-diyl, and anthr-1,8-diyl), phenanthrylene (including all isomeric forms, e.g., phenanthr-1,2-diyl, phenanthr-1,3-diyl, and phenanthr-1,8-diyl), pyrenylene (including all isomeric forms, e.g., pyren-1,2-diyl, pyren-1,3-diyl, and pyren-1,8-diyl), biphenylene (including all isomeric forms, e.g., biphen-2,3-diyl, biphen-3,4′-diyl, and biphen-4,4′-diyl), and terphenylene (including all isomeric forms, e.g., terphen-2,3-diyl, terphen-3,4′-diyl, and terphen-4,4′-diyl). Arylene also refers to bicyclic or tricyclic carbon rings, where one of the rings is aromatic and the others of which may be saturated, partially unsaturated, or aromatic, for example, dihydronaphthylene (including all isomeric forms, e.g., dihydronaphth-1,2-diyl and dihydronaphth-1,8-diyl), indenylene (including all isomeric forms, e.g., inden-1,2-diyl, inden-1,5-diyl, and inden-1,7-diyl), indanylene (including all isomeric forms, e.g., indan-1,2-diyl, indan-1,5-diyl, and indan-1,7-diyl), or tetrahydronaphthylene (tetralinylene) (including all isomeric forms, e.g., tetrahydronaphth-1,2-diyl, tetrahydronaphth-1,5-diyl, and tetrahydronaphth-1,8-diyl). In certain embodiments, arylene is optionally substituted with one or more substituents Q as described herein.

The term “aralkyl” or “arylalkyl” refers to a monovalent alkyl group substituted with one or more aryl groups. In certain embodiments, the aralkyl has from 7 to 30 (C₇₋₃₀), from 7 to 20 (C₇₋₂₀), or from 7 to 16 (C₇₋₁₆) carbon atoms. Examples of aralkyl groups include, but are not limited to, benzyl, phenylethyl (including all isomeric forms, e.g., 1-phenylethyl and 2-phenylethyl), and phenylpropyl (including all isomeric forms, e.g., 1-phenylpropyl, 2-phenylpropyl, and 3-phenylpropyl). In certain embodiments, the aralkyl is optionally substituted with one or more substituents Q as described herein.

The term “aralkylene” or “arylalkylene” refers to a divalent alkyl group substituted with one or more aryl groups. In certain embodiments, the aralkylene has from 7 to 30 (C₇₋₃₀), from 7 to 20 (C₇₋₂₀), or from 7 to 16 (C₇₋₁₆) carbon atoms. Examples of aralkylene groups include, but are not limited to, benzylene (including all isomeric forms, e.g., phenylmethdiyl), phenylethylene (including all isomeric forms, e.g., 2-phenyl-ethan-1,1-diyl and 2-phenyl-ethan-1,2-diyl), and phenylpropylene (including all isomeric forms, e.g., 3-phenyl-propan-1,1-diyl, 3-phenyl-propan-1,2-diyl, and 3-phenyl-propan-1,3-diyl). In certain embodiments, the aralkylene is optionally substituted with one or more substituents Q as described herein.

The term “heteroaryl” refers to a monovalent monocyclic aromatic group or monovalent polycyclic aromatic group that contain at least one aromatic ring, wherein at least one aromatic ring contains one or more heteroatoms, each independently selected from O, S, and N, in the ring. For a heteroaryl group containing a heteroaromatic ring and a nonaromatic heterocyclic ring, the heteroaryl group is not bonded to the rest of a molecule through its nonaromatic heterocyclic ring. Each ring of a heteroaryl group can contain one or two O atoms, one or two S atoms, and/or one to four N atoms; provided that the total number of heteroatoms in each ring is four or less and each ring contains at least one carbon atom. In certain embodiments, the heteroaryl has from 5 to 20, from 5 to 15, or from 5 to 10 ring atoms. In one embodiment, the heteroaryl is monocyclic. Examples of monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. In another embodiment, the heteroaryl is bicyclic. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyrindyl (including all isomeric forms, e.g., furo[2,3-b]pyridinyl, furo[2,3-c]pyridinyl, furo[3,2-b]pyridinyl, furo[3,2-c]pyridinyl, furo[3,4-b]pyridinyl, and furo[3,4-c]pyridinyl), imidazopyridinyl (including all isomeric forms, e.g., imidazo[1,2-a]pyridinyl, imidazo[4,5-b]pyridinyl, and imidazo[4,5-c]pyridinyl), imidazothiazolyl (including all isomeric forms, e.g., imidazo[2,1-b]thiazolyl and imidazo[4,5-d]thiazolyl), indazolyl, indolizinyl, indolyl, isobenzofuranyl, isobenzothienyl (i.e., benzo[c]thienyl), isoindolyl, isoquinolinyl, naphthyridinyl (including all isomeric forms, e.g., 1,5-naphthyridinyl, 1,6-naphthyridinyl, 1,7-naphthyridinyl, and 1,8-naphthyridinyl), oxazolopyridinyl (including all isomeric forms, e.g., oxazolo[4,5-b]pyridinyl, oxazolo[4,5-c]pyridinyl, oxazolo[5,4-b]pyridinyl, and oxazolo[5,4-c]pyridinyl), phthalazinyl, pteridinyl, purinyl, pyrrolopyridyl (including all isomeric forms, e.g., pyrrolo[2,3-b]pyridinyl, pyrrolo[2,3-c]pyridinyl, pyrrolo[3,2-b]pyridinyl, and pyrrolo[3,2-c]pyridinyl), quinolinyl, quinoxalinyl, quinazolinyl, thiadiazolopyrimidyl (including all isomeric forms, e.g., [1,2,5]thiadiazolo[3,4-d]pyrimidinyl and [1,2,3]thiadiazolo[4,5-d]pyrimidinyl), and thienopyridyl (including all isomeric forms, e.g., thieno[2,3-b]pyridinyl, thieno[2,3-c]pyridinyl, thieno[3,2-b]pyridinyl, and thieno[3,2-c]pyridinyl). In yet another embodiment, the heteroaryl is tricyclic. Examples of tricyclic heteroaryl groups include, but are not limited to, acridinyl, benzindolyl, carbazolyl, dibenzofuranyl, perimidinyl, phenanthrolinyl, phenanthridinyl (including all isomeric forms, e.g., 1,5-phenanthrolinyl, 1,6-phenanthrolinyl, 1,7-phenanthrolinyl, 1,9-phenanthrolinyl, and 2,10-phenanthrolinyl), phenarsazinyl, phenazinyl, phenothiazinyl, phenoxazinyl, and xanthenyl. In certain embodiments, the heteroaryl is optionally substituted with one or more substituents Q as described herein.

The terms “heteroarylene” and “heteroarenediyl” are used interchangeably herein in reference to a divalent monocyclic aromatic group or divalent polycyclic aromatic group that contains at least one aromatic ring, wherein at least one aromatic ring contains one or more heteroatoms in the ring, each of which is independently selected from O, S, and N. For a heteroarylene group containing a heteroaromatic ring and a nonaromatic heterocyclic ring, the heteroarylene group is not bonded to the rest of a molecule via its nonaromatic heterocyclic ring. Each ring of a heteroarylene group can contain one or two O atoms, one or two S atoms, and/or one to four N atoms, provided that the total number of heteroatoms in each ring is four or less and each ring contains at least one carbon atom. In certain embodiments, the heteroarylene has from 5 to 20, from 5 to 15, or from 5 to 10 ring atoms. Examples of monocyclic heteroarylene groups include, but are not limited to, furandiyl, imidazoldiyl, isothiazoldiyl, isoxazoldiyl, oxadiazoldiyl, oxazoldiyl, pyrazindiyl, pyrazoldiyl, pyridazindiyl, pyridindiyl, pyrimidindiyl, pyrroldiyl, thiadiazoldiyl, thiazoldiyl, thiendiyl, tetrazoldiyl, triazinediyl, and triazoldiyl. Examples of bicyclic heteroarylene groups include, but are not limited to, benzofurandiyl, benzimidazoldiyl, benzoisoxazoldiyl, benzopyrandiyl, benzothiadiazoldiyl, benzothiazoldiyl, benzothiendiyl, benzotriazoldiyl, benzoxazoldiyl, furopyridindiyl (including all isomeric forms, e.g., furo[2,3-b]pyridindiyl, furo[2,3-c]pyridindiyl, furo[3,2-b]pyridindiyl, furo[3,2-c]-pyridindiyl, furo[3,4-b]pyridindiyl, and furo[3,4-c]pyridindiyl), imidazopyridindiyl (including all isomeric forms, e.g., imidazo[1,2-a]pyridindiyl, imidazo[4,5-b]pyridindiyl, and imidazo[4,5-c]-pyridindiyl), imidazothiazoldiyl (including all isomeric forms, e.g., imidazo[2,1-b]thiazoldiyl and imidazo[4,5-d]thiazoldiyl), indazoldiyl, indolizindiyl, indoldiyl, isobenzofurandiyl, isobenzothiendiyl (i.e., benzo[c]thiendiyl), isoindoldiyl, isoquinolindiyl, naphthyridindiyl (including all isomeric forms, e.g., 1,5-naphthyridindiyl, 1,6-naphthyridindiyl, 1,7-naphthyridindiyl, and 1,8-naphthyridindiyl), oxazolopyridindiyl (including all isomeric forms, e.g., oxazolo[4,5-b]pyridindiyl, oxazolo[4,5-c]pyridindiyl, oxazolo[5,4-b]pyridindiyl, and oxazolo[5,4-c]pyridindiyl), phthalazindiyl, pteridindiyl, purindiyl, pyrrolopyridindiyl (including all isomeric forms, e.g., pyrrolo[2,3-b]pyridindiyl, pyrrolo[2,3-c]pyridindiyl, pyrrolo[3,2-b]-pyridindiyl, and pyrrolo[3,2-c]pyridindiyl), quinolindiyl, quinoxalindiyl, quinazolindiyl, thiadiazolopyrimidindiyl (including all isomeric forms, e.g., [1,2,5]thiadiazolo[3,4-d]-pyrimidindiyl and [1,2,3]thiadiazolo[4,5-d]pyrimidindiyl), and thienopyridindiyl (including all isomeric forms, e.g., thieno[2,3-b]pyridindiyl, thieno[2,3-c]pyridindiyl, thieno[3,2-b]pyridindiyl, and thieno[3,2-c]pyridindiyl). Examples of tricyclic heteroarylene groups include, but are not limited to, acridindiyl, benzindoldiyl, carbazoldiyl, dibenzofurandiyl, perimidindiyl, phenanthrolindiyl (including all isomeric forms, e.g., 1,5-phenanthrolindiyl, 1,6-phenanthrolindiyl, 1,7-phenanthrolindiyl, 1,9-phenanthrolindiyl, and 2,10-phenanthrolindiyl), phenanthridindiyl, phenarsazindiyl, phenazindiyl, phenothiazindiyl, phenoxazindiyl, and xanthendiyl. In certain embodiments, heteroarylene is optionally substituted with one or more substituents Q as described herein.

The term “heterocyclyl” or “heterocyclic” refers to a monovalent monocyclic non-aromatic ring system or monovalent polycyclic ring system that contains at least one non-aromatic ring, wherein one or more of the non-aromatic ring atoms are heteroatoms, each independently selected from O, S, and N; and the remaining ring atoms are carbon atoms. For a heterocyclyl group containing a heteroaromatic ring and a nonaromatic heterocyclic ring, the heterocyclyl group is not bonded to the rest of a molecule through the heteroaromatic ring. In certain embodiments, the heterocyclyl or heterocyclic group has from 3 to 20, from 3 to 15, from 3 to 10, from 3 to 8, from 4 to 7, or from 5 to 6 ring atoms. In certain embodiments, the heterocyclyl is a monocyclic, bicyclic, tricyclic, or tetracyclic ring system, which may be fused or bridged, and in which nitrogen or sulfur atoms may be optionally oxidized, nitrogen atoms may be optionally quaternized, and some rings may be partially or fully saturated, or aromatic. The heterocyclyl may be attached to the main structure at any heteroatom or carbon atom which results in the creation of a stable compound. Examples of heterocyclyls and heterocyclic groups include, but are not limited to, azepinyl, benzodioxanyl, benzodioxolyl, benzofuranonyl, chromanyl, decahydroisoquinolinyl, dihydrobenzofuranyl, dihydrobenzisothiazolyl, dihydrobenzisoxazinyl (including all isomeric forms, e.g., 1,4-dihydrobenzo[d][1,3]oxazinyl, 3,4-dihydrobenzo[c][1,2]-oxazinyl, and 3,4-dihydrobenzo[d][1,2]oxazinyl), dihydrobenzothienyl, dihydroisobenzofuranyl, dihydrobenzo[c]thienyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isochromanyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, thiochromanyl, tetrahydroquinolinyl, and 1,3,5-trithianyl. In certain embodiments, the heterocyclyl is optionally substituted with one or more substituents Q as described herein.

The term “heterocyclylene” refers to a divalent monocyclic non-aromatic ring system or divalent polycyclic ring system that contains at least one non-aromatic ring, wherein one or more of the non-aromatic ring atoms are heteroatoms independently selected from O, S, and N; and the remaining ring atoms are carbon atoms. For a heterocyclylene group containing a heteroaromatic ring and a nonaromatic heterocyclic ring, the heterocyclylene group has at least one bond to the rest of a molecule via its nonaromatic heterocyclic ring. In certain embodiments, the heterocyclylene group has from 3 to 20, from 3 to 15, from 3 to 10, from 3 to 8, from 4 to 7, or from 5 to 6 ring atoms. In certain embodiments, the heterocyclylene is a monocyclic, bicyclic, tricyclic, or tetracyclic ring system, which may be fused or bridged, and in which nitrogen or sulfur atoms may be optionally oxidized, nitrogen atoms may be optionally quaternized, and some rings may be partially or fully saturated, or aromatic. The heterocyclylene may be attached to the main structure at any heteroatom or carbon atom which results in the creation of a stable compound. Examples of such heterocyclylene groups include, but are not limited to, azepindiyl, benzodioxandiyl, benzodioxoldiyl, benzofuranondiyl, chromandiyl, decahydroisoquinolindiyl, dihydrobenzofurandiyl, dihydrobenzisothiazoldiyl, dihydrobenzisoxazindiyl (including all isomeric forms, e.g., 1,4-dihydrobenzo[d][1,3]oxazindiyl, 3,4-dihydrobenzo[c][1,2]oxazindiyl, and 3,4-dihydrobenzo[d][1,2]oxazindiyl), dihydrobenzothiendiyl, dihydroisobenzofurandiyl, dihydrobenzo[c]thiendiyl, dihydrofurdiyl, dihydroisoindoldiyl, dihydropyrandiyl, dihydropyrazoldiyl, dihydropyrazindiyl, dihydropyridindiyl, dihydropyrimidindiyl, dihydropyrroldiyl, dioxolandiyl, 1,4-dithiandiyl, furanondiyl, imidazolidindiyl, imidazolindiyl, indolindiyl, isochromandiyl, isoindolindiyl, isothiazolidindiyl, isoxazolidindiyl, morpholindiyl, octahydroindoldiyl, octahydroisoindoldiyl, oxazolidinondiyl, oxazolidindiyl, oxirandiyl, piperazindiyl, piperidindiyl, 4-piperidondiyl, pyrazolidindiyl, pyrazolindiyl, pyrrolidindiyl, pyrrolindiyl, quinuclidindiyl, tetrahydrofurdiyl, tetrahydroisoquinolindiyl, tetrahydropyrandiyl, tetrahydrothiendiyl, thiamorpholindiyl, thiazolidindiyl, thiochromandiyl, tetrahydroquinolindiyl, and 1,3,5-trithiandiyl. In certain embodiments, the heterocyclylene is optionally substituted with one or more substituents Q as described herein.

The term “halogen”, “halide,” or “halo” refers to fluoro, chloro, bromo, and/or iodo.

The term “optionally substituted” is intended to mean that a group or substituent, such as an alkyl, heteroalkyl, alkylene, heteroalkylene, alkenyl, alkenylene, heteroalkenylene, alkynyl, alkynylene, heteroalkynylene, cycloalkyl, cycloalkylene, aryl, arylene, aralkyl, aralkylene, heteroaryl, heteroarylene, heterocyclyl, or heterocyclylene group, may be substituted with one or more, in one embodiment, one, two, three, or four, substituents Q, each of which is independently selected from, e.g., (a) deuterium (—D), cyano (—CN), halo, imino (═NH), nitro (—NO₂), and oxo (═O); (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{b})OR^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl optionally substituted with one or more, in one embodiment, one, two, three, or four, substituents Q^{a}. As used herein, all groups that can be substituted are “optionally substituted.”

In one embodiment, each Q^{a} is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OP(O)(OR^{f})OR^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, “optically active” and “enantiomerically active” refer to a collection of molecules, which has an enantiomeric excess of no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, an optically active compound comprises about 95% or more of one enantiomer and about 5% or less of the other enantiomer based on the total weight of the enantiomeric mixture in question. In certain embodiments, an optically active compound comprises about 98% or more of one enantiomer and about 2% or less of the other enantiomer based on the total weight of the enantiomeric mixture in question. In certain embodiments, an optically active compound comprises about 99% or more of one enantiomer and about 1% or less of the other enantiomer based on the total weight of the enantiomeric mixture in question.

In describing an optically active compound, the prefixes R and S are used to denote the absolute configuration of the compound about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the compound, R and S.

The term “isotopically enriched” refers to a compound that contains an unnatural proportion of an isotope at one or more of the atoms that constitute such a compound. In certain embodiments, an isotopically enriched compound contains unnatural proportions of one or more isotopes, including, but not limited to, hydrogen (H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an isotopically enriched compound is in a stable form, that is, non-radioactive. In certain embodiments, an isotopically enriched compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an isotopically enriched compound is in an unstable form, that is, radioactive. In certain embodiments, an isotopically enriched compound contains unnatural proportions of one or more isotopes, including, but not limited to, tritium (3H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I), iodine-125 (¹²⁵I), iodine-129 (¹²⁹I), and iodine-131 (¹³¹I). It will be understood that, in a compound as provided herein, any hydrogen can be ²H, as example, or any carbon can be ¹³C, as example, or any nitrogen can be ¹⁵N, as example, or any oxygen can be ¹⁸O, as example, where feasible according to the judgment of one of ordinary skill in the art.

The term “isotopic enrichment” refers to the percentage of incorporation of a less prevalent isotope (e.g., D for deuterium or hydrogen-2) of an element at a given position in a molecule in the place of a more prevalent isotope (e.g., ¹H for protium or hydrogen-1) of the element. As used herein, when an atom at a particular position in a molecule is designated as a particular less prevalent isotope, it is understood that the abundance of that isotope at that position is substantially greater than its natural abundance.

The term “isotopic enrichment factor” refers the ratio between the isotopic abundance in an isotopically enriched compound and the natural abundance of a specific isotope.

The term “hydrogen” or the symbol “H” refers to the composition of naturally occurring hydrogen isotopes, which include protium (¹H), deuterium (²H or D), and tritium (³H), in their natural abundances. Protium is the most common hydrogen isotope having a natural abundance of more than 99.98%. Deuterium is a less prevalent hydrogen isotope having a natural abundance of about 0.0156%.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156% on average, deuterium enrichment at any position in a compound synthesized using non-enriched starting materials is about 0.0156% on average. As used herein, when a particular position in an isotopically enriched compound is designated as having deuterium, it is understood that the abundance of deuterium at that position in the compound is substantially greater than its natural abundance (0.0156%).

The term “carbon” or the symbol “C” refers to the composition of naturally occurring carbon isotopes, which include carbon-12 (¹²C) and carbon-13 (¹³C) in their natural abundances. Carbon-12 is the most common carbon isotope having a natural abundance of more than 98.89%. Carbon-13 is a less prevalent carbon isotope having a natural abundance of about 1.11%.

The term “carbon-13 enrichment” or “¹³C enrichment” refers to the percentage of incorporation of carbon-13 at a given position in a molecule in the place of carbon. For example, carbon-13 enrichment of 10% at a given position means that 10% of molecules in a given sample contain carbon-13 at the specified position. Because the naturally occurring distribution of carbon-13 is about 1.11% on average, carbon-13 enrichment at any position in a compound synthesized using non-enriched starting materials is about 1.11% on average. As used herein, when a particular position in an isotopically enriched compound is designated as having carbon-13, it is understood that the abundance of carbon-13 at that position in the compound is substantially greater than its natural abundance (1.11%).

The terms “substantially pure” and “substantially homogeneous” mean, when referred to a substance, sufficiently homogeneous to appear free of readily detectable impurities as determined by a standard analytical method used by one of ordinary skill in the art, including, but not limited to, thin layer chromatography (TLC), gel electrophoresis, high performance liquid chromatography (HPLC), gas chromatography (GC), nuclear magnetic resonance (NMR), and mass spectrometry (MS); or sufficiently pure such that further purification would not detectably alter the physical, chemical, biological, and/or pharmacological properties, such as enzymatic and biological activities, of the substance. In certain embodiments, “substantially pure” or “substantially homogeneous” refers to a collection of molecules, wherein at least about 95%, at least about 96%, at least about 97%, at least about 98%, at least about 99%, or at least about 99.5% by weight of the molecules are a single compound, including a single enantiomer, a racemic mixture, or a mixture of enantiomers, as determined by standard analytical methods. As used herein, when an atom at a particular position in an isotopically enriched molecule is designated as a particular less prevalent isotope, a molecule that contains other than the designated isotope at the specified position is an impurity with respect to the isotopically enriched compound. Thus, for a deuterated compound that has an atom at a particular position designated as deuterium, a compound that contains a protium at the same position is an impurity.

The term “solvate” refers to a complex or aggregate formed by one or more molecules of a solute, e.g., a compound provided herein, and one or more molecules of a solvent, which are present in a stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

For a divalent group described herein, no orientation is implied by the direction in which the divalent group is presented. For example, unless a particular orientation is specified, the formula —C(O)NH— represents both —C(O)NH— and —NHC(O)—.

The phrase “an enantiomer, a mixture of enantiomers, a mixture of two or more diastereomers, a tautomer, a mixture of two or more tautomers, or an isotopic variant thereof; or a pharmaceutically acceptable salt, solvate, hydrate, or prodrug thereof” has the same meaning as the phrase “(i) an enantiomer, a mixture of enantiomers, a mixture of two or more diastereomers, a tautomer, a mixture of two or more tautomers, or an isotopic variant of the compound referenced therein; (ii) a pharmaceutically acceptable salt, solvate, hydrate, or prodrug of the compound referenced therein; or (iii) a pharmaceutically acceptable salt, solvate, hydrate, or prodrug of an enantiomer, a mixture of enantiomers, a mixture of two or more diastereomers, a tautomer, a mixture of two or more tautomers, or an isotopic variant of the compound referenced therein.”

### Compounds

In one embodiment, provided herein is a compound of Formula (I):

R^{T}-L-R^{E} (I)

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
L is a linker;
R^{E} is an E3 ubiquitin ligase binding moiety; and
R^{T} is a moiety of the structure of any one of Formulae (i) to (xv):

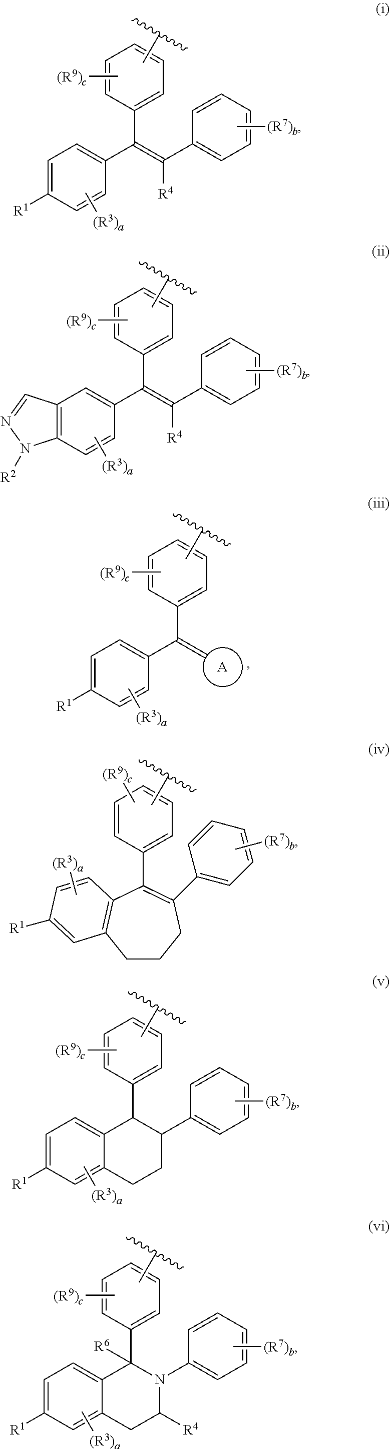

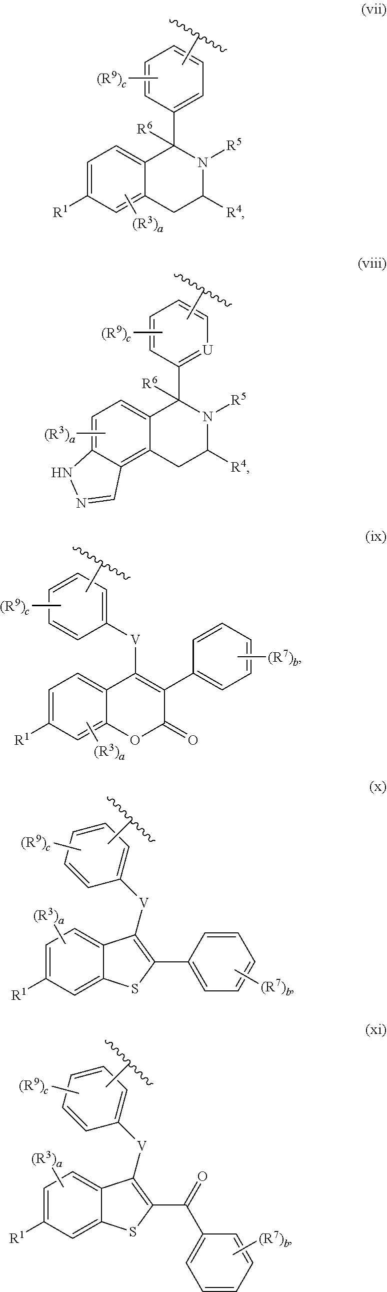

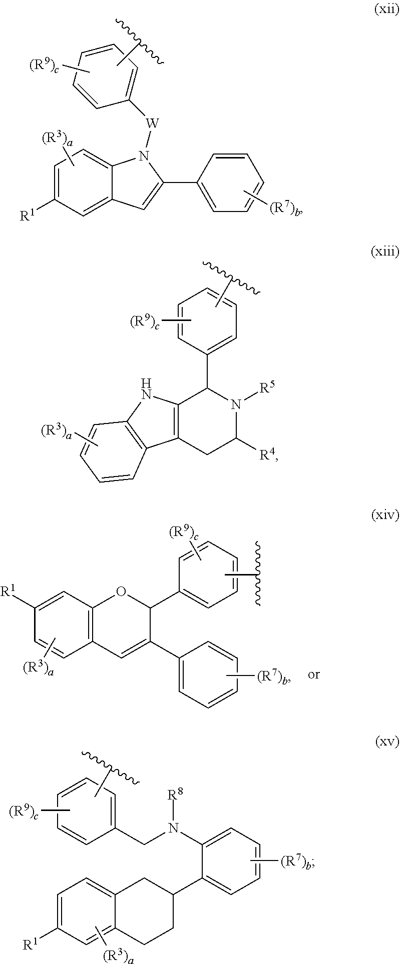

wherein:
ring A is C₃₋₁₀ cycloalkylene or heterocyclylene;
U is —CH═ or —N═;
each V is independently —O—, —S—, —N(R^{1a})—, —C(O)—, or C₁₋₆ alkylene;
W is —O—, —N(R^{1a})—, —C(O)—, or C₁₋₆ alkylene;
each R¹ is independently hydrogen, deuterium, halo, —C(O)OR^{1a}, —OR^{1a}, or —NR^{1b}R^{1c};
each R², R⁵, and R⁸ is independently (i) hydrogen; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c};
each R³, R⁷, and R⁹ is independently (i) hydrogen, deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each R⁴ and R⁶ is independently hydrogen, deuterium, C₁₋₆ alkyl, or C₁₋₆ heteroalkyl;
each a and c is independently an integer of 0, 1, 2, 3, or 4;
each b is independently an integer of 0, 1, 2, 3, 4, or 5; and
each R^{1a}, R^{1b}, R^{1c}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
wherein each alkyl, alkylene, heteroalkyl, alkenyl, alkynyl, cycloalkyl, cycloalkylene, aryl, aralkyl, heteroaryl, heterocyclyl, and heterocyclylene is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{b})OR^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR)NR^{f}R^{g}, —OC(S)R^{e}, —OC(S)OR^{e}, —OC(S)NR^{f}R^{g}, —OP(O)(OR^{f})OR^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In one embodiment, in Formula I,
ring A is C₃₋₁₀ cycloalkylene;
U is —CH═ or —N=;
each V is independently —O—, —C(O)—, or C₁₋₆ alkylene;
W is —C(O)— or C₁₋₆ alkylene;
each R¹ is independently hydrogen, deuterium, halo, —C(O)OR^{1a}, —OR^{1a}, or —NR^{1b}R^{1c};
R² and R⁸ are each independently hydrogen or C₁₋₆ alkyl;
each R³, R⁷, and R⁹ is independently halo, C₁₋₆ alkyl, C₁₋₆ heteroalkyl, or —OR^{1a};
each R⁴ and R⁶ is independently hydrogen, deuterium, C₁₋₆ alkyl, or C₁₋₆ heteroalkyl;
each R⁵ is independently C₁₋₆ alkyl, C₁₋₆ heteroalkyl, or C₇₋₁₅ aralkyl;
each a and c is independently an integer of 0, 1, 2, or 3;
each b is independently an integer of 0, 1, 2, 3, or 4; and
each R^{1a}, R^{1b}, and R^{1c} is as defined herein;
wherein each alkyl, alkylene, heteroalkyl, cycloalkylene, and aralkyl is optionally substituted with one or more substituents Q.

In another embodiment, in Formula I,
ring A is cyclohexan-1,1-diyl, bicyclo[3.3.1]nonan-9,9-diyl, or adamantan-2,2-diyl;
U is —CH═ or —N=;
each V is independently —O—, —C(O)—, or methanediyl;
W is —C(O)— or methanediyl;
each R¹ is independently hydrogen, fluoro, iodo, —CO₂H, —OH, or —NH₂;
R² is hydrogen or methyl;
each R³ is independently fluoro, chloro, hydroxyl, or methyl;
each R⁴ is independently hydrogen, methyl, ethyl, or 2-chloroethyl;
each R⁵ is independently 2,2,2-trifluoroethyl, 2,2-difluoropropyl, 2,2-difluoro-3-hydroxylpropyl, isobutyl, 2-fluoroisobutyl, or benzyl;
each R⁶ is independently hydrogen or methyl;
each R⁷ is independently fluoro, chloro, bromo, methyl, trifluoromethyl, ethyl, 2,2-filuoropropyl, isopropyl, isobutyl, t-butyl, cyclopropylmethyl, hydroxyl, methoxy, or trifluoromethoxy;
R⁸ is hydrogen or ethyl;
each R⁹ is independently fluoro, methyl, or methoxy;
each a and c is independently an integer of 0, 1, or 2; and
each b is independently an integer of 0, 1, 2, or 3.

In another embodiment, provided herein is a compound of Formula (II):

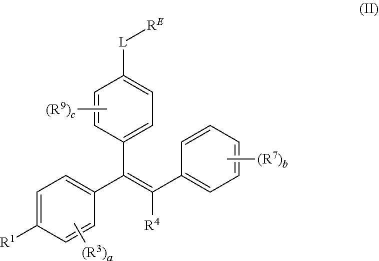

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁴, R⁷, R⁹, R^{E}, L, a, b, and c are each as defined herein.

In one embodiment, in Formula (i) or (II),
R¹ is hydrogen, deuterium, halo, —C(O)OR^{1a}, —OR^{1a}, or —NR^{1b}R^{1c};
each R³, R⁷, and R⁹ is independently halo, C₁₋₆ alkyl, C₁₋₆ heteroalkyl, or —OR^{1a};
R⁴ is C₁₋₆ alkyl or C₁₋₆ heteroalkyl;
a and c are each independently an integer of 0, 1, 2, or 3;
b is an integer of 0, 1, 2, 3, or 4; and
each R^{1a}, R^{1b}, and R^{1c} is as defined herein;
wherein each alkyl and heteroalkyl is optionally substituted with one or more substituents Q.

In another embodiment, in Formula (i) or (II),
R¹ is independently hydrogen, fluoro, iodo, —CO₂H, —OH, or —NH₂;
each R³ is independently fluoro, chloro, hydroxyl, or methyl;
R⁴ is ethyl or 2-chloroethyl;
each R⁷ is independently fluoro, chloro, bromo, methyl, trifluoromethyl, ethyl, 2,2-fluoropropyl, isopropyl, isobutyl, t-butyl, cyclopropylmethyl, hydroxyl, methoxy, or trifluoromethoxy;
each R⁹ is independently fluoro, methyl, or methoxy;
a and c are each independently an integer of 0, 1, or 2; and
b is an integer of 0, 1, 2, or 3.

In yet another embodiment, in Formula (i) or (II),
R¹ is independently hydrogen, —CO₂H, or —OH;
each R³ is independently fluoro, chloro, hydroxyl, or methyl;
R⁴ is ethyl or 2-chloroethyl;
each R⁷ is independently fluoro, chloro, bromo, methyl, trifluoromethyl, ethyl, 2,2-filuoropropyl, isopropyl, isobutyl, t-butyl, cyclopropylmethyl, hydroxyl, methoxy, or trifluoromethoxy;
each R⁹ is independently fluoro, methyl, or methoxy;
a and c are each independently an integer of 0, 1, or 2; and
b is an integer of 0, 1, 2, or 3.

In still another embodiment, the moiety of Formula (i) in a compound provided herein is:

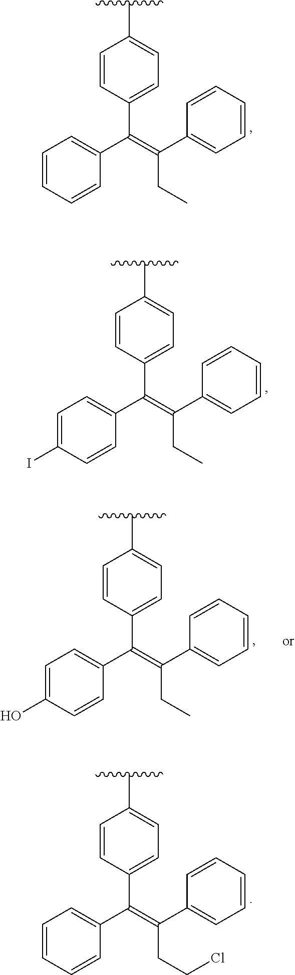 [SMILES: *c1ccc(/C(=C(/CC)c2ccccc2)c2ccccc2)cc1 ; *c1ccc(/C(=C(/CC)c2ccccc2)c2ccc(I)cc2)cc1 ; *c1ccc(/C(=C(/CC)c2ccccc2)c2ccc(O)cc2)cc1 ; *c1ccc(/C(=C(/CCCl)c2ccccc2)c2ccccc2)cc1]

In yet another embodiment, provided herein is a compound of Formula (III):

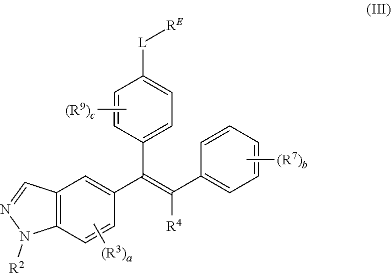

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R², R³, R⁴, R⁷, R⁹, R^{E}, L, a, b, and c are each as defined herein.

In one embodiment, in Formula (ii) or (III),
R² is hydrogen or C₁₋₆ alkyl;
each R³, R⁷, and R⁹ is independently halo, C₁₋₆ alkyl, C₁₋₆ heteroalkyl, or —OR^{1a}; wherein R^{1a} is as defined herein;
R⁴ is C₁₋₆ alkyl or C₁₋₆ heteroalkyl;
a and c are each independently an integer of 0, 1, 2, or 3; and
b is an integer of 0, 1, 2, 3, or 4;
wherein each alkyl and heteroalkyl is optionally substituted with one or more substituents Q.

In another embodiment, in Formula (ii) or (III),
R² is hydrogen or methyl;
each R³ is independently fluoro, chloro, hydroxyl, or methyl;
R⁴ is ethyl or 2-chloroethyl;
each R⁷ is independently fluoro, chloro, bromo, methyl, trifluoromethyl, ethyl, 2,2-filuoropropyl, isopropyl, isobutyl, t-butyl, cyclopropylmethyl, hydroxyl, methoxy, or trifluoromethoxy;
each R⁹ is independently fluoro, methyl, or methoxy;
a and c are each independently an integer of 0, 1, or 2; and
b is an integer of 0, 1, 2, or 3.

In yet another embodiment, the moiety of Formula (ii) in a compound provided herein is:

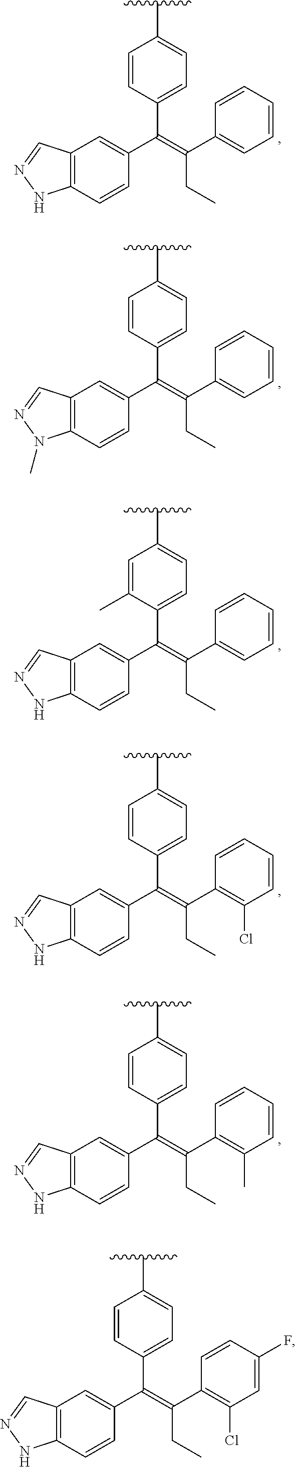 [SMILES: *c1ccc(/C(=C(/CC)c2ccccc2)c2ccc3[nH]ncc3c2)cc1 ; *c1ccc(/C(=C(/CC)c2ccccc2)c2ccc3c(cnn3C)c2)cc1 ; *c1ccc(/C(=C(/CC)c2ccccc2)c2ccc3[nH]ncc3c2)c(C)c1 ; *c1ccc(/C(=C(/CC)c2ccccc2Cl)c2ccc3[nH]ncc3c2)cc1 ; *c1ccc(/C(=C(/CC)c2ccccc2C)c2ccc3[nH]ncc3c2)cc1 ; *c1ccc(/C(=C(/CC)c2ccc(F)cc2Cl)c2ccc3[nH]ncc3c2)cc1]

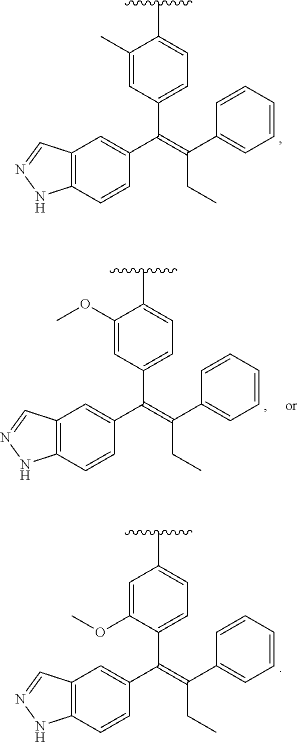 [SMILES: *c1ccc(/C(=C(/CC)c2ccccc2)c2ccc3[nH]ncc3c2)cc1C ; *c1ccc(/C(=C(/CC)c2ccccc2)c2ccc3[nH]ncc3c2)cc1OC ; *c1ccc(/C(=C(/CC)c2ccccc2)c2ccc3[nH]ncc3c2)c(OC)c1]

In yet another embodiment, provided herein is a compound of Formula (IV):

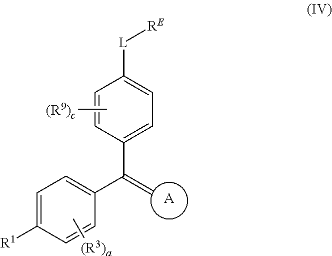

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof, wherein ring A, R¹, R³, R⁹, R^{E}, L, a, and c are each as defined herein.

In one embodiment, in Formula (iii) or (IV),
ring A is C₃₋₁₀ cycloalkylene;
R¹ is hydrogen, deuterium, halo, —C(O)OR^{1a}, —OR^{1a}, or —NR^{1b}R^{1c};
each R³ and R⁹ is independently halo, C₁₋₆ alkyl, C₁₋₆ heteroalkyl, or —OR^{1a};
a and c are each independently an integer of 0, 1, 2, or 3; and
each R^{1a}, R^{1b}, and R^{1c} is as defined herein;
wherein each alkyl, heteroalkyl, and cycloalkylene is optionally substituted with one or more substituents Q.

In another embodiment, in Formula (iii) or (IV),
ring A is cyclohexan-1,1-diyl, bicyclo[3.3.1]nonan-9,9-diyl, or adamantan-2,2-diyl;
R¹ is hydrogen, fluoro, iodo, —CO₂H, —OH, or —NH₂;
each R³ is independently fluoro, chloro, hydroxyl, or methyl;
each R⁹ is independently fluoro, methyl, or methoxy; and
a and c are each independently an integer of 0, 1, or 2.

In yet another embodiment, in Formula (iii) or (IV),
ring A is cyclohexan-1,1-diyl, bicyclo[3.3.1]nonan-9,9-diyl, or adamantan-2,2-diyl;
R¹ is hydrogen, —CO₂H, or —OH;
each R³ is independently fluoro, chloro, hydroxyl, or methyl;
each R⁹ is independently fluoro, methyl, or methoxy; and
a and c are each independently an integer of 0, 1, or 2.

In still another embodiment, the moiety of Formula (iii) in a compound provided herein is:

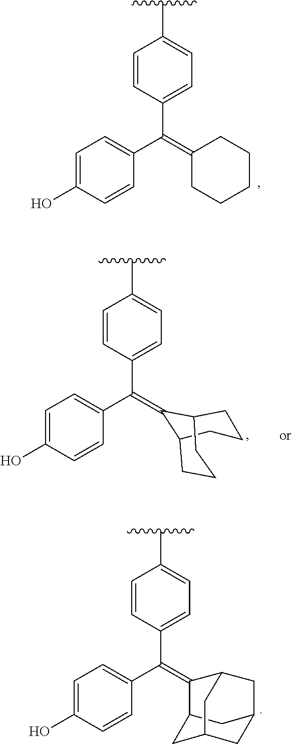 [SMILES: *c1ccc(C(=C2CCCCC2)c2ccc(O)cc2)cc1 ; *c1ccc(C(=C2C3CCCC2CCC3)c2ccc(O)cc2)cc1 ; *c1ccc(C(=C2C3CC4CC(C3)CC2C4)c2ccc(O)cc2)cc1]

In yet another embodiment, provided herein is a compound of Formula (V):

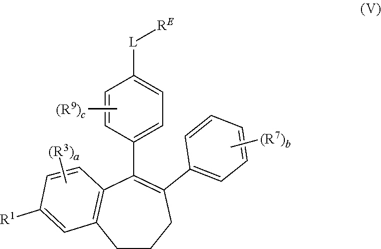

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R⁹, R^{E}, L, a, b, and c are each as defined herein.

In one embodiment, in Formula (iv) or (V),
R¹ is hydrogen, deuterium, halo, —C(O)OR^{1a}, —OR^{1a}, or —NR^{1b}R^{1c};
each R³, R⁷, and R⁹ is independently halo, C₁₋₆ alkyl, C₁₋₆ heteroalkyl, or —OR^{1a};
a and c are each independently an integer of 0, 1, 2, or 3;
b is an integer of 0, 1, 2, 3, or 4; and
each R^{1a}, R^{1b}, and R^{1c} is as defined herein;
wherein each alkyl and heteroalkyl is optionally substituted with one or more substituents Q.

In another embodiment, in Formula (iv) or (V),
R¹ is independently hydrogen, fluoro, iodo, —CO₂H, —OH, or —NH₂;
each R³ is independently fluoro, chloro, hydroxyl, or methyl;
each R⁷ is independently fluoro, chloro, bromo, methyl, trifluoromethyl, ethyl, 2,2-filuoropropyl, isopropyl, isobutyl, t-butyl, cyclopropylmethyl, hydroxyl, methoxy, or trifluoromethoxy;
each R⁹ is independently fluoro, methyl, or methoxy;
a and c are each independently an integer of 0, 1, or 2; and
b is an integer of 0, 1, 2, or 3.

In yet another embodiment, in Formula (iv) or (V),
R¹ is independently hydrogen, —CO₂H, or —OH;
each R³ is independently fluoro, chloro, hydroxyl, or methyl;
each R⁷ is independently fluoro, chloro, bromo, methyl, trifluoromethyl, ethyl, 2,2-filuoropropyl, isopropyl, isobutyl, t-butyl, cyclopropylmethyl, hydroxyl, methoxy, or trifluoromethoxy;
each R⁹ is independently fluoro, methyl, or methoxy;
a and c are each independently an integer of 0, 1, or 2; and
b is an integer of 0, 1, 2, or 3.

In yet another embodiment, the moiety of Formula (iv) in a compound provided herein is:

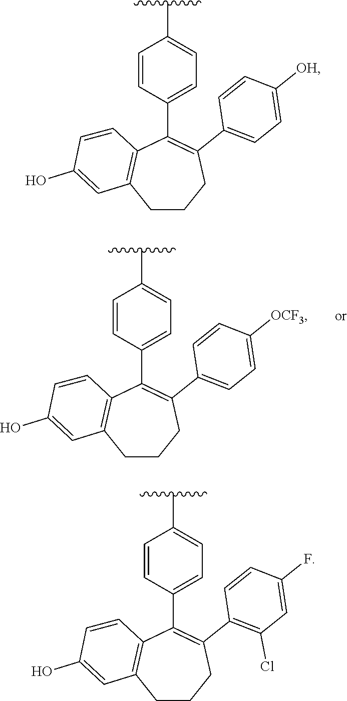

In still another embodiment, the moiety of Formula (iv) in a compound provided herein is:

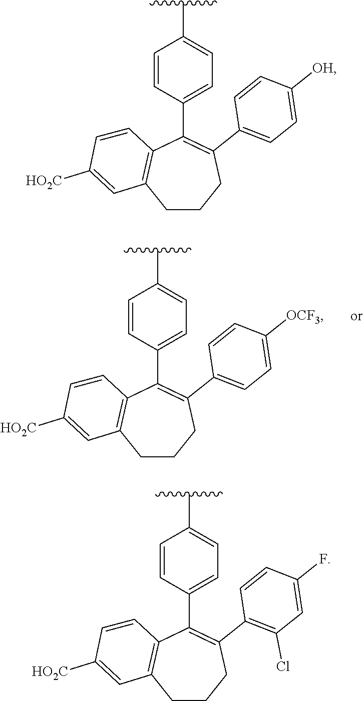

In yet another embodiment, provided herein is a compound of Formula (VI):

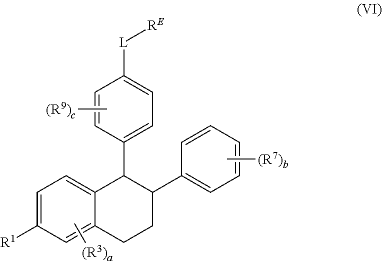

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R⁹, R^{E}, L, a, b, and c are each as defined herein.

In one embodiment, in Formula (v) or (VI),
R¹ is hydrogen, deuterium, halo, —C(O)OR^{1a}, —OR^{1a}, or —NR^{1b}R^{1c};
each R³, R⁷, and R⁹ is independently halo, C₁₋₆ alkyl, C₁₋₆ heteroalkyl, or —OR^{1a};
a and c are each independently an integer of 0, 1, 2, or 3;
b is an integer of 0, 1, 2, 3, or 4; and
each R^{1a}, R^{1b}, and R^{1c} is as defined herein;
wherein each alkyl and heteroalkyl is optionally substituted with one or more substituents Q.

In another embodiment, in Formula (v) or (VI),
R¹ is independently hydrogen, fluoro, iodo, —CO₂H, —OH, or —NH₂;
each R³ is independently fluoro, chloro, hydroxyl, or methyl;
each R⁷ is independently fluoro, chloro, bromo, methyl, trifluoromethyl, ethyl, 2,2-filuoropropyl, isopropyl, isobutyl, t-butyl, cyclopropylmethyl, hydroxyl, methoxy, or trifluoromethoxy;
each R⁹ is independently fluoro, methyl, or methoxy;
a and c are each independently an integer of 0, 1, or 2; and
b is an integer of 0, 1, 2, or 3.

In yet another embodiment, in Formula (v) or (VI),
R¹ is independently hydrogen, —CO₂H, or —OH;
each R³ is independently fluoro, chloro, hydroxyl, or methyl;
each R⁷ is independently fluoro, chloro, bromo, methyl, trifluoromethyl, ethyl, 2,2-filuoropropyl, isopropyl, isobutyl, t-butyl, cyclopropylmethyl, hydroxyl, methoxy, or trifluoromethoxy;
each R⁹ is independently fluoro, methyl, or methoxy;
a and c are each independently an integer of 0, 1, or 2; and
b is an integer of 0, 1, 2, or 3.

In still another embodiment, the moiety of Formula (v) in a compound provided herein is:

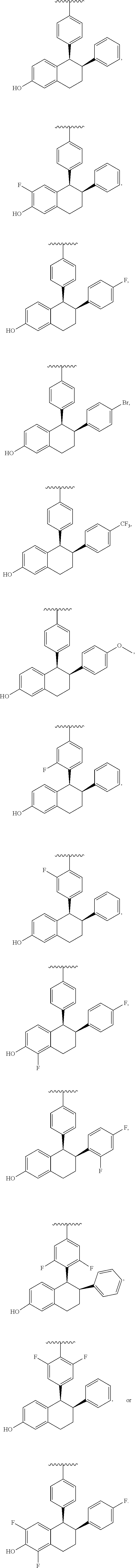

In yet another embodiment, provided herein is a compound of Formula (VII):

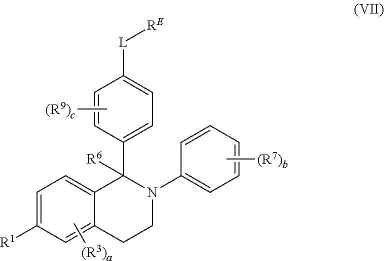

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁶, R⁷, R⁹, R^{E}, L, a, b, and c are each as defined herein.

In one embodiment, in Formula (vi) or (VII),
R¹ is hydrogen, deuterium, halo, —C(O)OR^{1a}, —OR^{1a}, or —NR^{1b}R^{1c};
each R³, R⁷, and R⁹ is independently halo, C₁₋₆ alkyl, C₁₋₆ heteroalkyl, or —OR^{1a};
R⁶ is hydrogen, deuterium, C₁₋₆ alkyl, or C₁₋₆ heteroalkyl;
a and c are each independently an integer of 0, 1, 2, or 3;
b is an integer of 0, 1, 2, 3, or 4; and
each R^{1a}, R^{1b}, and R^{1c} is as defined herein;
wherein each alkyl and heteroalkyl is optionally substituted with one or more substituents Q.

In another embodiment, in Formula (vi) or (VII),
R¹ is independently hydrogen, fluoro, iodo, —CO₂H, —OH, or —NH₂;
each R³ is independently fluoro, chloro, hydroxyl, or methyl;
R⁶ is hydrogen or methyl;
each R⁷ is independently fluoro, chloro, bromo, methyl, trifluoromethyl, ethyl, 2,2-filuoropropyl, isopropyl, isobutyl, t-butyl, cyclopropylmethyl, hydroxyl, methoxy, or trifluoromethoxy;
each R⁹ is independently fluoro, methyl, or methoxy;
a and c are each independently an integer of 0, 1, or 2; and
b is an integer of 0, 1, 2, or 3.

In yet another embodiment, in Formula (vi) or (VII),
R¹ is independently hydrogen, —CO₂H, or —OH;
each R³ is independently fluoro, chloro, hydroxyl, or methyl;
R⁶ is hydrogen or methyl;
each R⁷ is independently fluoro, chloro, bromo, methyl, trifluoromethyl, ethyl, 2,2-filuoropropyl, isopropyl, isobutyl, t-butyl, cyclopropylmethyl, hydroxyl, methoxy, or trifluoromethoxy;
each R⁹ is independently fluoro, methyl, or methoxy;
a and c are each independently an integer of 0, 1, or 2; and
b is an integer of 0, 1, 2, or 3.

In still another embodiment, the moiety of Formula (vi) in a compound provided herein is:

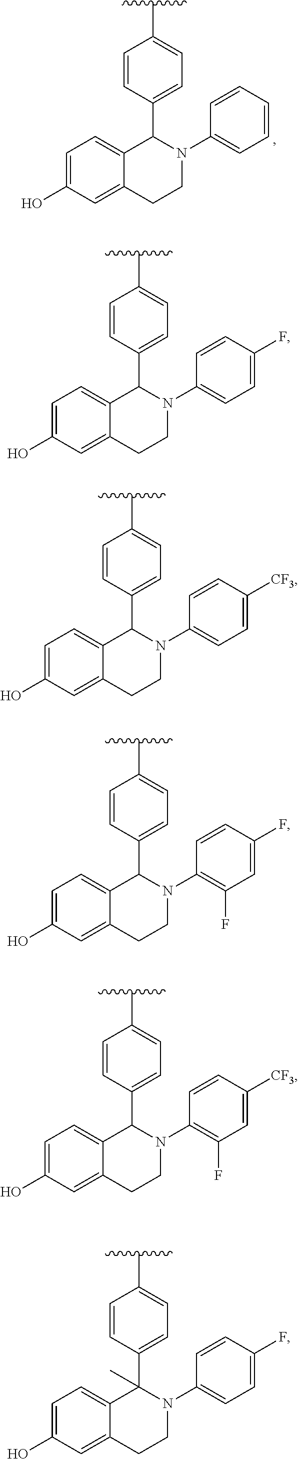

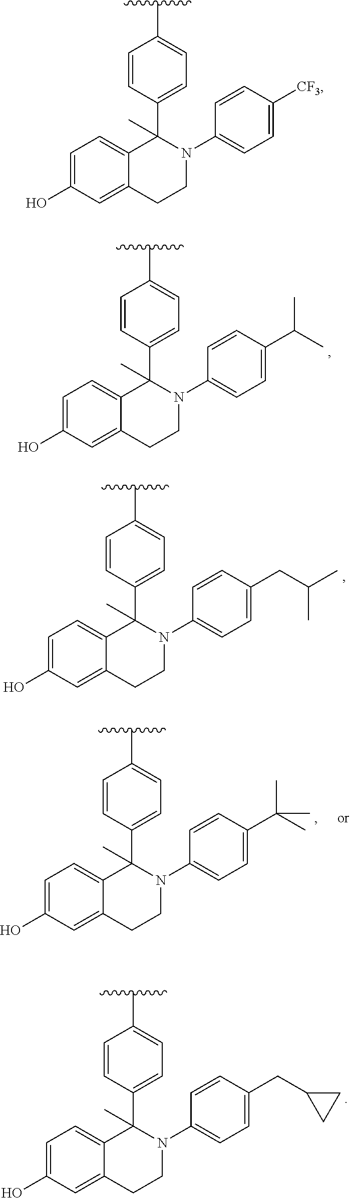

In yet another embodiment, provided herein is a compound of Formula (VIII):

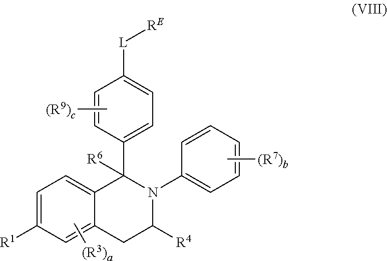

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁴, R⁵, R⁶, R⁹, R^{E}, L, a, and c are each as defined herein.

In one embodiment, in Formula (vii) or (VIII),
R¹ is hydrogen, deuterium, halo, —C(O)OR^{1a}, —OR^{1a}, or —NR^{1b}R^{1c};
each R³ and R⁹ is independently halo, C₁₋₆ alkyl, C₁₋₆ heteroalkyl, or —OR^{1a};
R⁴ and R⁶ are each independently hydrogen, deuterium, C₁₋₆ alkyl, or C₁₋₆ heteroalkyl;
R⁵ is C₁₋₆ alkyl, C₁₋₆ heteroalkyl, or C₇₋₁₅ aralkyl;
a and c are each independently an integer of 0, 1, 2, or 3; and
each R^{1a}, R^{1b}, and R^{1c} is as defined herein;
wherein each alkyl, heteroalkyl, and aralkyl is optionally substituted with one or more substituents Q.

In another embodiment, in Formula (vii) or (VIII),
R¹ is independently hydrogen, fluoro, iodo, —CO₂H, —OH, or —NH₂;
each R³ is independently fluoro, chloro, hydroxyl, or methyl;
R⁴ is hydrogen, methyl, ethyl, or 2-chloroethyl;
R⁵ is 2,2,2-trifluoroethyl, 2,2-difluoropropyl, 2,2-difluoro-3-hydroxylpropyl, isobutyl, 2-fluoroisobutyl, or benzyl;
R⁶ is hydrogen or methyl;
each R⁹ is independently fluoro, methyl, or methoxy; and
a and c are each independently an integer of 0, 1, or 2.

In yet another embodiment, in Formula (vii) or (VIII),
R¹ is independently hydrogen, —CO₂H, or —OH;
each R³ is independently fluoro, chloro, hydroxyl, or methyl;
R⁴ is hydrogen, methyl, ethyl, or 2-chloroethyl;
R⁵ is 2,2,2-trifluoroethyl, 2,2-difluoropropyl, 2,2-difluoro-3-hydroxylpropyl, isobutyl, 2-fluoroisobutyl, or benzyl;
R⁶ is hydrogen or methyl;
each R⁹ is independently fluoro, methyl, or methoxy; and
a and c are each independently an integer of 0, 1, or 2.

In still another embodiment, the moiety of Formula (vii) in a compound provided herein is:

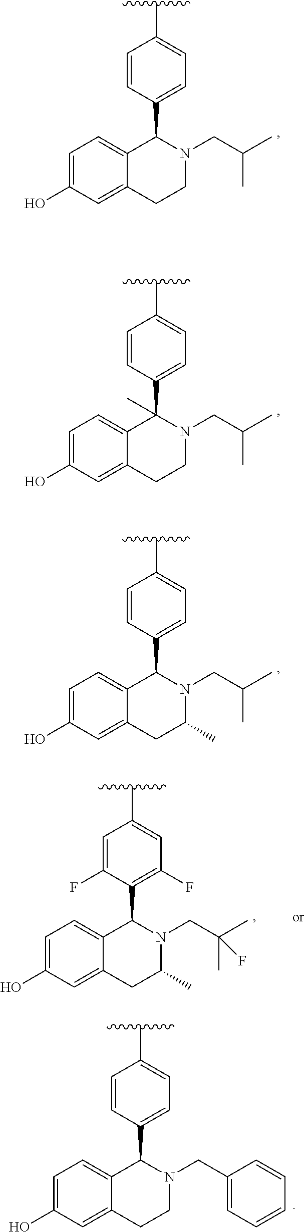

In yet another embodiment, provided herein is a compound of Formula (IX):

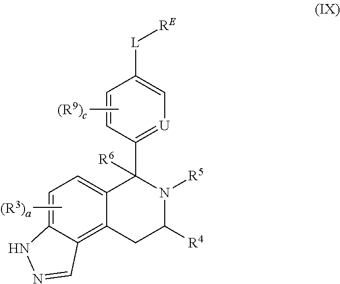

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R³, R⁴, R⁵, R⁶, R⁹, R^{E}, L, U, a, and c are each as defined herein.

In one embodiment, in Formula (viii) or (IX),
each U is independently —CH═ or —N=;
each R³ and R⁹ is independently halo, C₁₋₆ alkyl, C₁₋₆ heteroalkyl, or —OR^{1a}; wherein R^{1a} is as defined herein;
R⁴ and R⁶ are each independently is hydrogen, deuterium, C₁₋₆ alkyl, or C₁₋₆ heteroalkyl;
R⁵ is C₁₋₆ alkyl, C₁₋₆ heteroalkyl, or C₇₋₁₅ aralkyl; and
a and c are each independently an integer of 0, 1, 2, or 3;
wherein each alkyl, heteroalkyl, and aralkyl is optionally substituted with one or more substituents Q.

In another embodiment, in Formula (viii) or (IX),
each U is independently —CH═ or —N═;
each R³ is independently fluoro, chloro, hydroxyl, or methyl;
R⁴ is hydrogen, methyl, ethyl, or 2-chloroethyl;
R⁵ is 2,2,2-trifluoroethyl, 2,2-difluoropropyl, 2,2-difluoro-3-hydroxylpropyl, isobutyl, 2-fluoroisobutyl, or benzyl;
R⁶ is hydrogen or methyl;
each R⁹ is independently fluoro, methyl, or methoxy; and
a and c are each independently an integer of 0, 1, or 2.

In yet another embodiment, the moiety of Formula (viii) in a compound provided herein is:

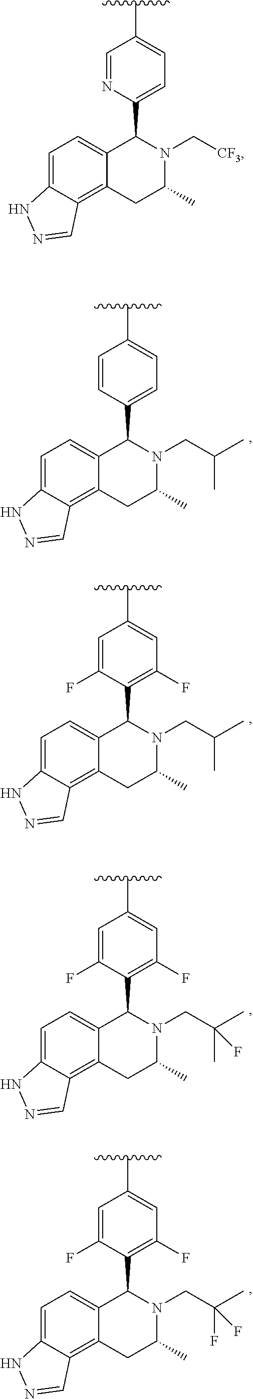 [SMILES: *c1ccc([C@@H]2c3ccc4[nH]ncc4c3C[C@@H](C)N2CC(F)(F)F)nc1 ; *c1ccc([C@@H]2c3ccc4[nH]ncc4c3C[C@@H](C)N2CC(C)C)cc1 ; *c1cc(F)c([C@@H]2c3ccc4[nH]ncc4c3C[C@@H](C)N2CC(C)C)c(F)c1 ; *c1cc(F)c([C@@H]2c3ccc4[nH]ncc4c3C[C@@H](C)N2CC(C)(C)F)c(F)c1 ; *c1cc(F)c([C@@H]2c3ccc4[nH]ncc4c3C[C@@H](C)N2CC(C)(F)F)c(F)c1]

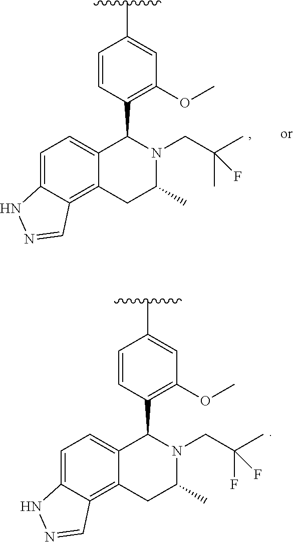 [SMILES: *c1ccc([C@@H]2c3ccc4[nH]ncc4c3C[C@@H](C)N2CC(C)(C)F)c(OC)c1 ; *c1ccc([C@@H]2c3ccc4[nH]ncc4c3C[C@@H](C)N2CC(C)(F)F)c(OC)c1]

In yet another embodiment, provided herein is a compound of Formula (X):

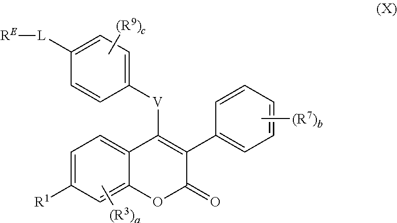

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R⁹, R^{E}, L, V, a, b, and c are each as defined herein.

In one embodiment, in Formula (ix) or (X),
V is —O—, —C(O)—, or C₁₋₆ alkylene;
R¹ is hydrogen, deuterium, halo, —C(O)OR^{1a}, —OR^{1a}, or —NR^{1b}R^{1c};
each R³, R⁷, and R⁹ is independently halo, C₁₋₆ alkyl, C₁₋₆ heteroalkyl, or —OR^{1a};
a and c are each independently an integer of 0, 1, 2, or 3;
b is an integer of 0, 1, 2, 3, or 4; and
each R^{1a}, R^{1b}, and R^{1c} is as defined herein;
wherein each alkyl, alkylene, and heteroalkyl is optionally substituted with one or more substituents Q.

In another embodiment, in Formula (ix) or (X),
V is —O—, —C(O)—, or methanediyl;
R¹ is independently hydrogen, fluoro, iodo, —CO₂H, —OH, or —NH₂;
each R³ is independently fluoro, chloro, hydroxyl, or methyl;
each R⁷ is independently fluoro, chloro, bromo, methyl, trifluoromethyl, ethyl, 2,2-filuoropropyl, isopropyl, isobutyl, t-butyl, cyclopropylmethyl, hydroxyl, methoxy, or trifluoromethoxy;
each R⁹ is independently fluoro, methyl, or methoxy;
a and c are each independently an integer of 0, 1, or 2; and
b is an integer of 0, 1, 2, or 3.

In yet another embodiment, in Formula (ix) or (X),
V is —O—, —C(O)—, or methanediyl;
R¹ is independently hydrogen, —CO₂H, or —OH;
each R³ is independently fluoro, chloro, hydroxyl, or methyl;
each R⁷ is independently fluoro, chloro, bromo, methyl, trifluoromethyl, ethyl, 2,2-filuoropropyl, isopropyl, isobutyl, t-butyl, cyclopropylmethyl, hydroxyl, methoxy, or trifluoromethoxy;
each R⁹ is independently fluoro, methyl, or methoxy;
a and c are each independently an integer of 0, 1, or 2; and
c is an integer of 0, 1, 2, or 3.

In yet another embodiment, the moiety of Formula (ix) in a compound provided herein is:

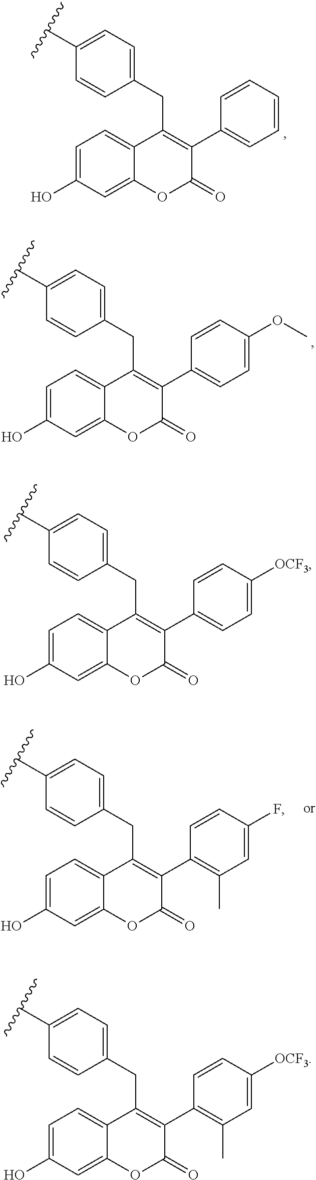 [SMILES: *c1ccc(Cc2c(-c3ccccc3)c(=O)oc3cc(O)ccc23)cc1 ; *c1ccc(Cc2c(-c3ccc(OC)cc3)c(=O)oc3cc(O)ccc23)cc1 ; *c1ccc(Cc2c(-c3ccc(OC(F)(F)F)cc3)c(=O)oc3cc(O)ccc23)cc1 ; *c1ccc(Cc2c(-c3ccc(F)cc3C)c(=O)oc3cc(O)ccc23)cc1 ; "or" ; *c1ccc(Cc2c(-c3ccc(OC(F)(F)F)cc3C)c(=O)oc3cc(O)ccc23)cc1]

In still another embodiment, the moiety of Formula (ix) in a compound provided herein is:

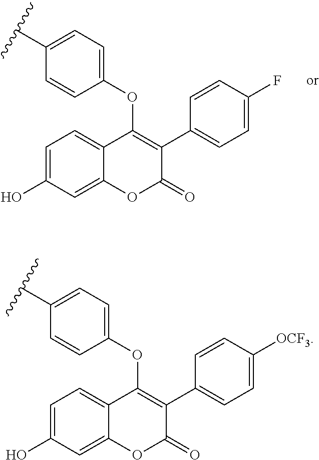 [SMILES: *c1ccc(Oc2c(-c3ccc(F)cc3)c(=O)oc3cc(O)ccc23)cc1 ; *c1ccc(Oc2c(-c3ccc(OC(F)(F)F)cc3)c(=O)oc3cc(O)ccc23)cc1]

In yet another embodiment, provided herein is a compound of Formula (XI):

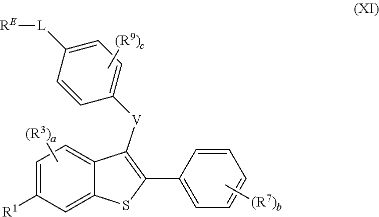

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R⁹, R^{E}, L, V, a, b, and c are each as defined herein.

In one embodiment, in Formula (x) or (XI),
V is —O—, —C(O)—, or C₁₋₆ alkylene;
R¹ is hydrogen, deuterium, halo, —C(O)OR^{1a}, —OR^{1a}, or —NR^{1b}R^{1c};
each R³, R⁷, and R⁹ is independently halo, C₁₋₆ alkyl, C₁₋₆ heteroalkyl, or —OR^{1a};
a and c are each independently an integer of 0, 1, 2, or 3;
b is an integer of 0, 1, 2, 3, or 4; and
each R^{1a}, R^{1b}, and R^{1c} is as defined herein;
wherein each alkyl, alkylene, and heteroalkyl is optionally substituted with one or more substituents Q.

In another embodiment, in Formula (x) or (XI),
V is —O—, —C(O)—, or methanediyl;
R¹ is independently hydrogen, fluoro, iodo, —CO₂H, —OH, or —NH₂;
each R³ is independently fluoro, chloro, hydroxyl, or methyl;
each R⁷ is independently fluoro, chloro, bromo, methyl, trifluoromethyl, ethyl, 2,2-filuoropropyl, isopropyl, isobutyl, t-butyl, cyclopropylmethyl, hydroxyl, methoxy, or trifluoromethoxy;
each R⁹ is independently fluoro, methyl, or methoxy;
a and c are each independently an integer of 0, 1, or 2; and
b is an integer of 0, 1, 2, or 3.

In yet another embodiment, in Formula (x) or (XI),
V is —O—, —C(O)—, or methanediyl;
R¹ is independently hydrogen, —CO₂H, or —OH;
each R³ is independently fluoro, chloro, hydroxyl, or methyl;
each R⁷ is independently fluoro, chloro, bromo, methyl, trifluoromethyl, ethyl, 2,2-filuoropropyl, isopropyl, isobutyl, t-butyl, cyclopropylmethyl, hydroxyl, methoxy, or trifluoromethoxy;
each R⁹ is independently fluoro, methyl, or methoxy;
a and c are each independently an integer of 0, 1, or 2; and
b is an integer of 0, 1, 2, or 3.

In yet another embodiment, the moiety of Formula (x) in a compound provided herein is:

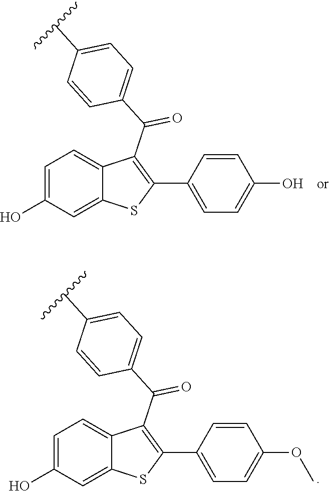 [SMILES: *c1ccc(C(=O)c2c(-c3ccc(O)cc3)sc3cc(O)ccc23)cc1 ; *c1ccc(C(=O)c2c(-c3ccc(OC)cc3)sc3cc(O)ccc23)cc1]

In still another embodiment, the moiety of Formula (x) in a compound provided herein is:

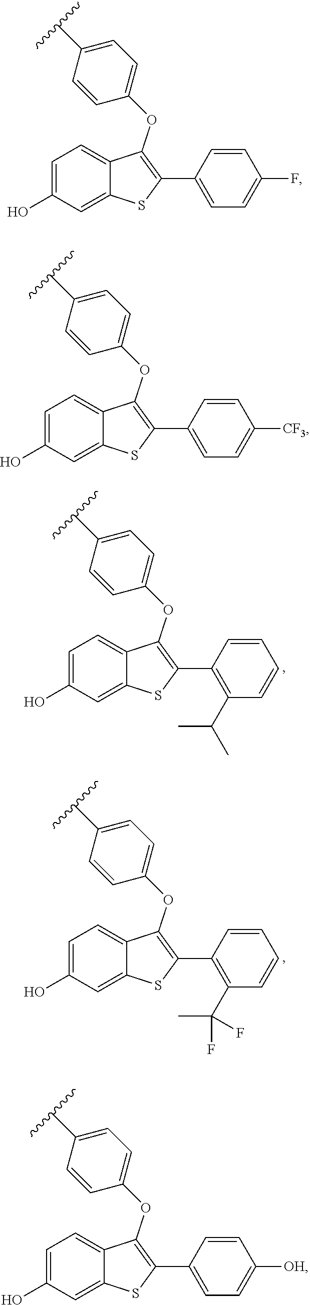 [SMILES: *c1ccc(Oc2c(-c3ccc(F)cc3)sc3cc(O)ccc23)cc1 ; *c1ccc(Oc2c(-c3ccc(C(F)(F)F)cc3)sc3cc(O)ccc23)cc1 ; *c1ccc(Oc2c(-c3ccccc3C(C)C)sc3cc(O)ccc23)cc1 ; *c1ccc(Oc2c(-c3ccccc3C(C)(F)F)sc3cc(O)ccc23)cc1 ; *c1ccc(Oc2c(-c3ccc(O)cc3)sc3cc(O)ccc23)cc1]

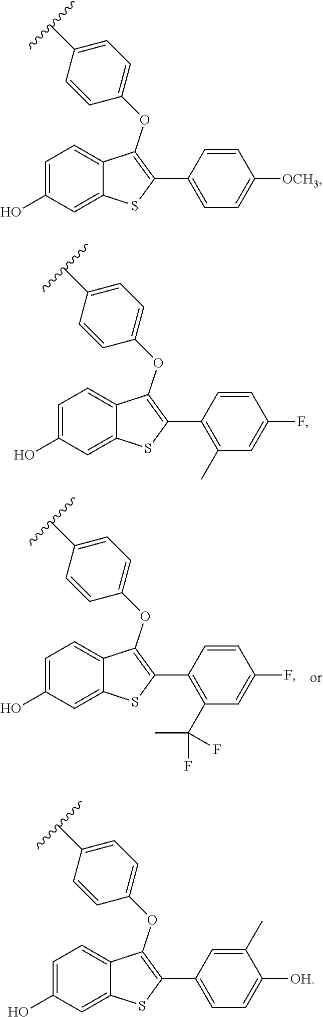 [SMILES: *c1ccc(Oc2c(-c3ccc(OC)cc3)sc3cc(O)ccc23)cc1 ; *c1ccc(Oc2c(-c3ccc(F)cc3C)sc3cc(O)ccc23)cc1 ; *c1ccc(Oc2c(-c3ccc(F)cc3C(C)(F)F)sc3cc(O)ccc23)cc1 ; *c1ccc(Oc2c(-c3ccc(O)c(C)c3)sc3cc(O)ccc23)cc1]

In yet another embodiment, provided herein is a compound of Formula (XII):

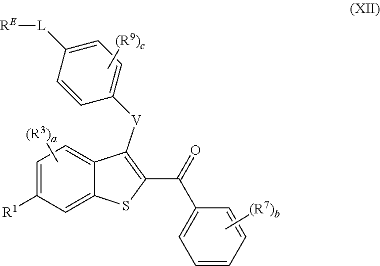

or an enantiomer, a mixture of enantiomers, a diastereomer, a mixture of two or more diastereomers, a tautomer, a mixture of two or more tautomers, or an isotopic variant thereof or a pharmaceutically acceptable salt, solvate, hydrate, or prodrug thereof; wherein R¹, R³, R⁷, R⁹, R^{E}, L, V, a, b, and c are each as defined herein.

In one embodiment, in Formula (xi) or (XII),
V is —O—, —C(O)—, or C₁₋₆ alkylene;
R¹ is hydrogen, deuterium, halo, —C(O)OR^{1a}, —OR^{1a}, or —NR^{1b}R^{1c};
each R³, R⁷, and R⁹ is independently halo, C₁₋₆ alkyl, C₁₋₆ heteroalkyl, or —OR^{1a};
a and c are each independently an integer of 0, 1, 2, or 3;
b is an integer of 0, 1, 2, 3, or 4; and
each R^{1a}, R^{1b}, and R^{1c} is as defined herein;
wherein each alkyl, alkylene, and heteroalkyl is optionally substituted with one or more substituents Q.

In another embodiment, in Formula (xi) or (XII),
V is —O—, —C(O)—, or methanediyl;
R¹ is independently hydrogen, fluoro, iodo, —CO₂H, —OH, or —NH₂;
each R³ is independently fluoro, chloro, hydroxyl, or methyl;
each R⁷ is independently fluoro, chloro, bromo, methyl, trifluoromethyl, ethyl, 2,2-filuoropropyl, isopropyl, isobutyl, t-butyl, cyclopropylmethyl, hydroxyl, methoxy, or trifluoromethoxy;
each R⁹ is independently fluoro, methyl, or methoxy;
a and c are each independently an integer of 0, 1, or 2; and
b is an integer of 0, 1, 2, or 3.

In yet another embodiment, in Formula (xi) or (XII),
V is —O—, —C(O)—, or methanediyl;
R¹ is independently hydrogen, —CO₂H, or —OH;
each R³ is independently fluoro, chloro, hydroxyl, or methyl;
each R⁷ is independently fluoro, chloro, bromo, methyl, trifluoromethyl, ethyl, 2,2-filuoropropyl, isopropyl, isobutyl, t-butyl, cyclopropylmethyl, hydroxyl, methoxy, or trifluoromethoxy;
each R⁹ is independently fluoro, methyl, or methoxy;
a and c are each independently an integer of 0, 1, or 2; and
b is an integer of 0, 1, 2, or 3.

In still another embodiment, the moiety of Formula (xi) in a compound provided herein is:

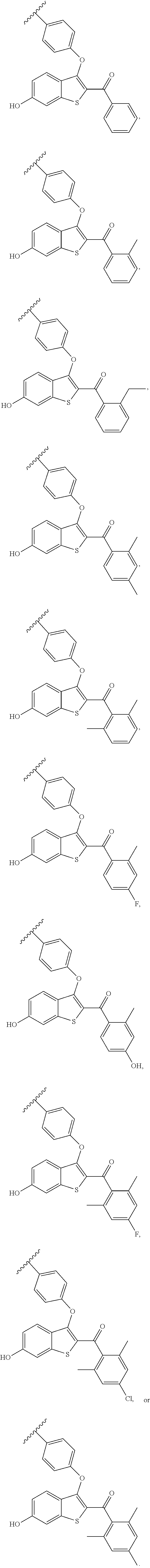

In yet another embodiment, provided herein is a compound of Formula (XIII):

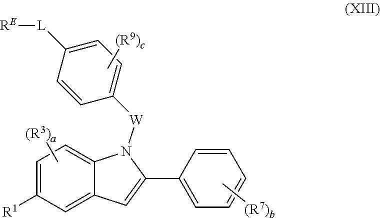

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R⁹, R^{E}, L, W, a, b, and c are each as defined herein.

In one embodiment, in Formula (xii) or (XIII),
W is —C(O)— or C₁₋₆ alkylene;
R¹ is hydrogen, deuterium, halo, —C(O)OR^{1a}, —OR^{1a}, or —NR^{1b}R^{1c};
each R³, R⁷, and R⁹ is independently halo, C₁₋₆ alkyl, C₁₋₆ heteroalkyl, or —OR^{1a};
a and c are each independently an integer of 0, 1, 2, or 3;
b is an integer of 0, 1, 2, 3, or 4; and
each R^{1a}, R^{1b}, and R^{1c} is as defined herein;
wherein each alkyl, alkylene, and heteroalkyl is optionally substituted with one or more substituents Q.

In another embodiment, in Formula (xii) or (XIII),
W is —C(O)— or methanediyl;
R¹ is independently hydrogen, fluoro, iodo, —CO₂H, —OH, or —NH₂;
each R³ is independently fluoro, chloro, hydroxyl, or methyl;
each R⁷ is independently fluoro, chloro, bromo, methyl, trifluoromethyl, ethyl, 2,2-filuoropropyl, isopropyl, isobutyl, t-butyl, cyclopropylmethyl, hydroxyl, methoxy, or trifluoromethoxy;
each R⁹ is independently fluoro, methyl, or methoxy;
a and c are each independently an integer of 0, 1, or 2; and
b is an integer of 0, 1, 2, or 3.

In yet another embodiment, in Formula (xii) or (XIII),
W is —C(O)— or methanediyl;
R¹ is independently hydrogen, —CO₂H, or —OH;
each R³ is independently fluoro, chloro, hydroxyl, or methyl;
each R⁷ is independently fluoro, chloro, bromo, methyl, trifluoromethyl, ethyl, 2,2-filuoropropyl, isopropyl, isobutyl, t-butyl, cyclopropylmethyl, hydroxyl, methoxy, or trifluoromethoxy;
each R⁹ is independently fluoro, methyl, or methoxy;
a and c are each independently an integer of 0, 1, or 2; and
b is an integer of 0, 1, 2, or 3.

In yet another embodiment, the moiety of Formula (xii) in a compound provided herein is

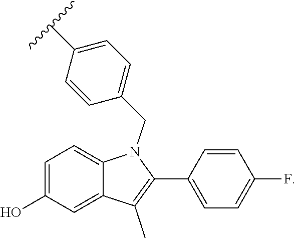 [SMILES: *c1ccc(Cn2c(-c3ccc(F)cc3)c(C)c3cc(O)ccc32)cc1]

In still another embodiment, the moiety of Formula (xii) in a compound provided herein is

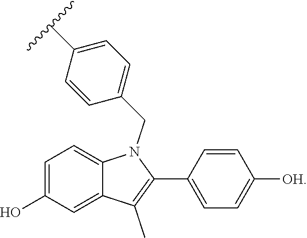 [SMILES: *c1ccc(Cn2c(-c3ccc(O)cc3)c(C)c3cc(O)ccc32)cc1]

In yet another embodiment, provided herein is a compound of Formula (XIV):

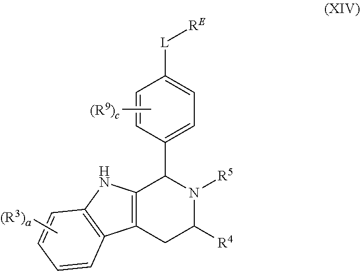

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R³, R⁴, R⁵, R⁹, R^{E}, L, a, and c are each as defined herein.

In one embodiment, in Formula (xiii) or (XIV),
each R³ and R⁹ is independently halo, C₁₋₆ alkyl, C₁₋₆ heteroalkyl, or —OR^{1a}; wherein R^{1a} is as defined herein;
R⁴ is hydrogen, deuterium, C₁₋₆ alkyl, or C₁₋₆ heteroalkyl;
R⁵ is C₁₋₆ alkyl, C₁₋₆ heteroalkyl, or C₇₋₁₅ aralkyl; and
a and c are each independently an integer of 0, 1, 2, or 3;
wherein each alkyl, heteroalkyl, and aralkyl is optionally substituted with one or more substituents Q.

In another embodiment, in Formula (xiii) or (XIV),
each R³ is independently fluoro, chloro, hydroxyl, or methyl;
R⁴ is hydrogen, methyl, ethyl, or 2-chloroethyl;
R⁵ is 2,2,2-trifluoroethyl, 2,2-difluoropropyl, 2,2-difluoro-3-hydroxylpropyl, isobutyl, 2-fluoroisobutyl, or benzyl;
each R⁹ is independently fluoro, methyl, or methoxy; and
a and c are each independently an integer of 0, 1, or 2.

In yet another embodiment, the moiety of Formula (xiii) in a compound provided herein is:

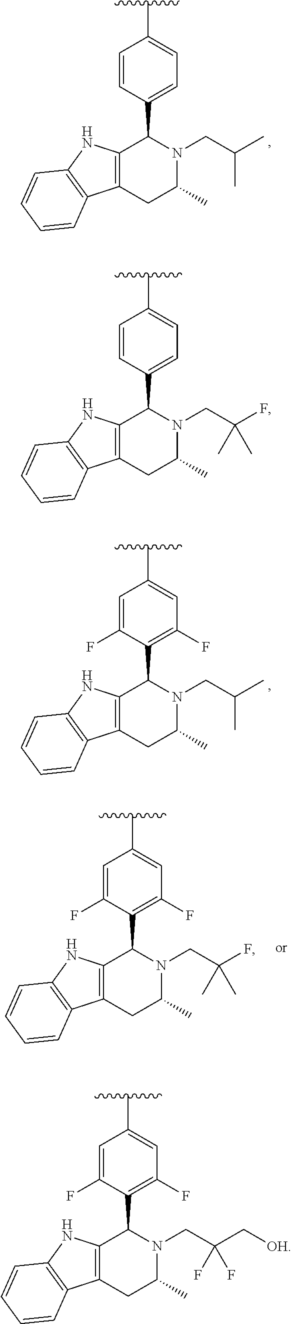 [SMILES: *c1ccc([C@@H]2c3[nH]c4ccccc4c3C[C@@H](C)N2CC(C)C)cc1 ; *c1ccc([C@@H]2c3[nH]c4ccccc4c3C[C@@H](C)N2CC(C)(C)F)cc1 ; *c1cc(F)c([C@@H]2c3[nH]c4ccccc4c3C[C@@H](C)N2CC(C)C)c(F)c1 ; *c1cc(F)c([C@@H]2c3[nH]c4ccccc4c3C[C@@H](C)N2CC(C)(C)F)c(F)c1 ; "or" ; *c1cc(F)c([C@@H]2c3[nH]c4ccccc4c3C[C@@H](C)N2CC(F)(F)CO)c(F)c1]

In yet another embodiment, provided herein is a compound of Formula (XV):

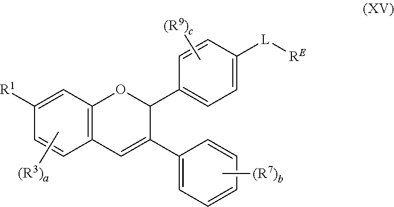

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R⁹, R^{E}, L, a, b, and c are each as defined herein.

In one embodiment, in Formula (xiv) or (XV),
R¹ is hydrogen, deuterium, halo, —C(O)OR^{1a}, —OR^{1a}, or —NR^{1b}R^{1c};
each R³, R⁷, and R⁹ is independently halo, C₁₋₆ alkyl, C₁₋₆ heteroalkyl, or —OR^{1a};
a and c are each independently an integer of 0, 1, 2, or 3;
b is an integer of 0, 1, 2, 3, or 4; and
each R^{1a}, R^{1b}, and R^{1c} is as defined herein;
wherein each alkyl and heteroalkyl is optionally substituted with one or more substituents Q.

In another embodiment, in Formula (xiv) or (XV),
R¹ is independently hydrogen, fluoro, iodo, —CO₂H, —OH, or —NH₂;
each R³ is independently fluoro, chloro, hydroxyl, or methyl;
each R⁷ is independently fluoro, chloro, bromo, methyl, trifluoromethyl, ethyl, 2,2-filuoropropyl, isopropyl, isobutyl, t-butyl, cyclopropylmethyl, hydroxyl, methoxy, or trifluoromethoxy;
each R⁹ is independently fluoro, methyl, or methoxy;
a and c are each independently an integer of 0, 1, or 2; and
b is an integer of 0, 1, 2, or 3.

In yet another embodiment, in Formula (xiv) or (XV),
R¹ is independently hydrogen, —CO₂H, or —OH;
each R³ is independently fluoro, chloro, hydroxyl, or methyl;
each R⁷ is independently fluoro, chloro, bromo, methyl, trifluoromethyl, ethyl, 2,2-filuoropropyl, isopropyl, isobutyl, t-butyl, cyclopropylmethyl, hydroxyl, methoxy, or trifluoromethoxy;
each R⁹ is independently fluoro, methyl, or methoxy;
a and c are each independently an integer of 0, 1, or 2; and
b is an integer of 0, 1, 2, or 3.

In still another embodiment, the moiety of Formula (xiv) in a compound provided herein is:

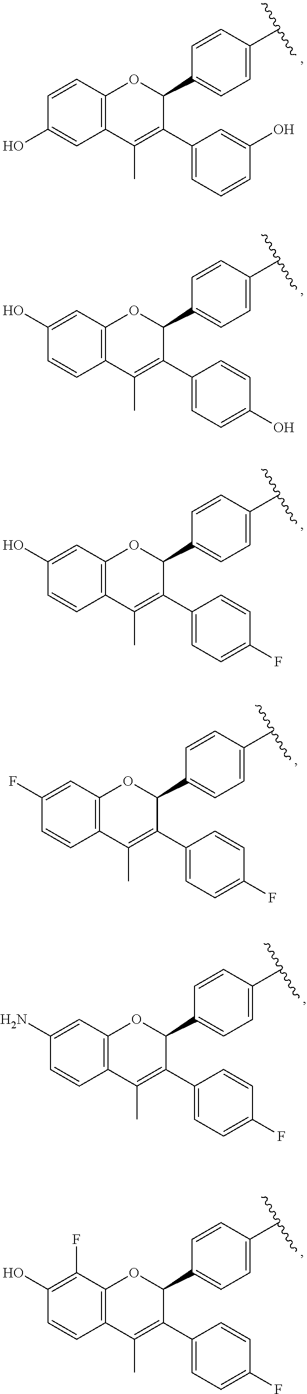 [SMILES: *c1ccc([C@@H]2Oc3ccc(O)cc3C(C)=C2c2cccc(O)c2)cc1 ; *c1ccc([C@@H]2Oc3cc(O)ccc3C(C)=C2c2ccc(O)cc2)cc1 ; *c1ccc([C@@H]2Oc3cc(O)ccc3C(C)=C2c2ccc(F)cc2)cc1 ; *c1ccc([C@@H]2Oc3cc(F)ccc3C(C)=C2c2ccc(F)cc2)cc1 ; *c1ccc([C@@H]2Oc3cc(N)ccc3C(C)=C2c2ccc(F)cc2)cc1 ; *c1ccc([C@@H]2Oc3c(ccc(O)c3F)C(C)=C2c2ccc(F)cc2)cc1]

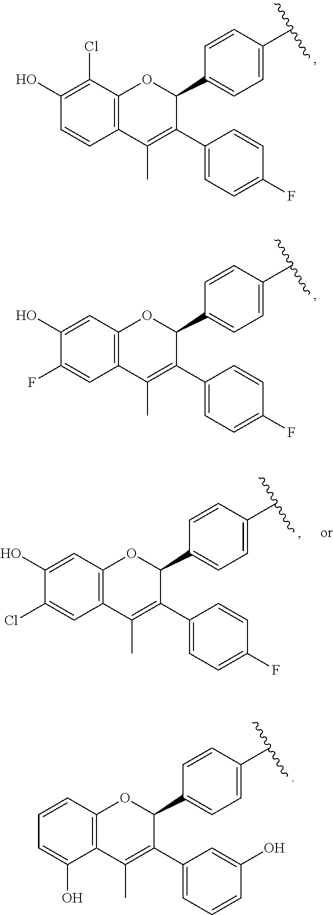 [SMILES: *c1ccc([C@@H]2Oc3c(ccc(O)c3Cl)C(C)=C2c2ccc(F)cc2)cc1 ; *c1ccc([C@@H]2Oc3cc(O)c(F)cc3C(C)=C2c2ccc(F)cc2)cc1 ; *c1ccc([C@@H]2Oc3cc(O)c(Cl)cc3C(C)=C2c2ccc(F)cc2)cc1 ; *c1ccc([C@@H]2Oc3cccc(O)c3C(C)=C2c2cccc(O)c2)cc1]

In still another embodiment, provided herein is a compound of Formula (XVI):

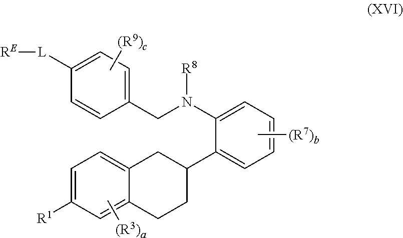

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R⁸, R⁹, R^{E}, L, a, b, and c are each as defined herein.

In one embodiment, in Formula (xv) or (XVI),
R¹ is hydrogen, deuterium, halo, —C(O)OR^{1a}, —OR^{1a}, or —NR^{1b}R^{1c};
each R³, R⁷, and R⁹ is independently halo, C₁₋₆ alkyl, C₁₋₆ heteroalkyl, or —OR^{1a};
R⁸ is hydrogen or C₁₋₆ alkyl;
a and c are each independently an integer of 0, 1, 2, or 3;
b is an integer of 0, 1, 2, 3, or 4; and
each R^{1a}, R^{1b}, and R^{1c} is as defined herein;
wherein each alkyl and heteroalkyl is optionally substituted with one or more substituents Q.

In another embodiment, in Formula (xv) or (XVI),
R¹ is independently hydrogen, fluoro, iodo, —CO₂H, —OH, or —NH₂;
each R³ is independently fluoro, chloro, hydroxyl, or methyl;
each R⁷ is independently fluoro, chloro, bromo, methyl, trifluoromethyl, ethyl, 2,2-filuoropropyl, isopropyl, isobutyl, t-butyl, cyclopropylmethyl, hydroxyl, methoxy, or trifluoromethoxy;
R⁸ is hydrogen or ethyl;
each R⁹ is independently fluoro, methyl, or methoxy;
a and c are each independently an integer of 0, 1, or 2; and
b is an integer of 0, 1, 2, or 3.

In yet another embodiment, in Formula (xv) or (XVI),
R¹ is independently hydrogen, —CO₂H, or —OH;
each R³ is independently fluoro, chloro, hydroxyl, or methyl;
each R⁷ is independently fluoro, chloro, bromo, methyl, trifluoromethyl, ethyl, 2,2-filuoropropyl, isopropyl, isobutyl, t-butyl, cyclopropylmethyl, hydroxyl, methoxy, or trifluoromethoxy;
R⁸ is hydrogen or ethyl;
each R⁹ is independently fluoro, methyl, or methoxy;
a and c are each independently an integer of 0, 1, or 2; and
b is an integer of 0, 1, 2, or 3.

In still another embodiment, the moiety of Formula (xv) in a compound provided herein is

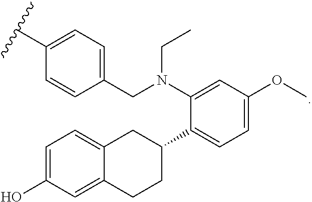 [SMILES: *c1ccc(CN(CC)c2cc(OC)ccc2[C@@H]2CCc3cc(O)ccc3C2)cc1]

In certain embodiments, R^{E} is a moiety of a cereblon (CRBN) E3 ligand, an inhibitors-of-apoptosis protein (IAP) E3 ligand, a mouse double minute 2 homolog (MDM2) E3 ligand, or a von Hippel-Lindau (VHL) E3 ligand.

In certain embodiments, R^{E} is a moiety of a CRBN E3 ligand.

In certain embodiments, R^{E} is a moiety having the structure of Formula (EC-I):

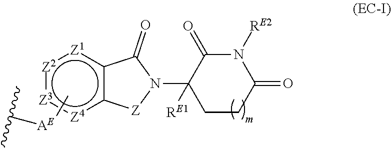

or an enantiomer, a mixture of enantiomers, a diastereomer, a mixture of two or more diastereomers, a tautomer, a mixture of two or more tautomers, or an isotopic variant thereof; wherein:
A^{E} is a bond, —O—, —N(R^{1b})—, —S—, C₁₋₆ alkylene, C₁₋₆ heteroalkylene, C₂₋₆ alkenylene, C₂₋₆ heteroalkenylene, C₂₋₆ alkynylene, C₂₋₆ heteroalkynylene, C₃₋₁₀ cycloalkylene, C₆₋₁₄ arylene, C₇₋₁₅ aralkylene, heteroarylene, heterocyclylene, C₁₋₆ heteroalkylene-C₆₋₁₄ arylene, C₁₋₆ heteroalkylene-heterocyclylene, or C₂₋₆ alkynylene-heterocyclylene;
Z is —CH₂— or —C(O)—;
one of Z¹, Z², Z³, and Z⁴ is —C═ and the remaining three of Z¹, Z², Z³, and Z⁴ are each independently —C(R^{E5})=; or Z¹ is a bond; one of Z², Z³, and Z⁴ is —C═, and the remaining two of Z², Z³, and Z⁴ are each independently —C(R^{E5})═ or —S—;
m is an integer of 0, 1, or 2;
R^{E1} is hydrogen, deuterium, halo, or C₁₋₆ alkyl;
R^{E2} is hydrogen or C₁₋₆ alkyl;
each R^{E4} is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each R^{E5} is independently hydrogen or R^{E4}; and
R^{1a}, R^{1b}, R^{1c} and R^{1d} are each as defined herein;
wherein each alkyl, heteroalkyl, alkylene, heteroalkylene, alkenyl, alkenylene, heteroalkenylene, alkynyl, alkynylene, heteroalkynylene, cycloalkyl, cycloalkylene, aryl, arylene, aralkyl, aralkylene, heteroaryl, heteroarylene, heterocyclyl, and heterocyclylene is optionally substituted with one or more, in one embodiment, one, two, three, or four, substituents Q.

In certain embodiments, R^{E} is a moiety having the structure of Formula (EC-II):

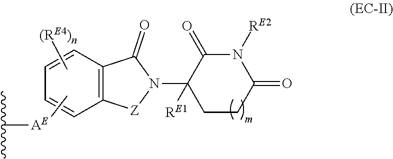

or an enantiomer, a mixture of enantiomers, a diastereomer, a mixture of two or more diastereomers, a tautomer, a mixture of two or more tautomers, or an isotopic variant thereof; wherein n is an integer of 0, 1, 2, or 3; and A^{E}, R^{E1}, R^{E2}, R^{E4}, Z, and m are each as defined herein.

In certain embodiments, R^{E} is a moiety having the structure of Formula (EC-III):

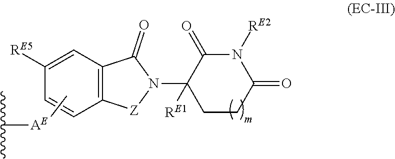

or an enantiomer, a mixture of enantiomers, a diastereomer, a mixture of two or more diastereomers, a tautomer, a mixture of two or more tautomers, or an isotopic variant thereof; wherein A^{E}, R^{E1}, R^{E2}, R^{E5}, Z, and m are each as defined herein. In one embodiment, R^{E5} is hydrogen or fluoro.

In certain embodiments, R^{E} is a moiety having the structure of Formula (EC-IV):

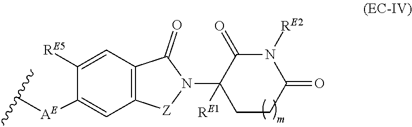

or an enantiomer, a mixture of enantiomers, a diastereomer, a mixture of two or more diastereomers, a tautomer, a mixture of two or more tautomers, or an isotopic variant thereof; wherein A^{E}, R^{E1}, R^{E2}, R^{E5}, Z, and m are each as defined herein. In one embodiment, R^{E5} is hydrogen or fluoro.

In certain embodiments, R^{E} is a moiety having the structure of Formula (EC-V):

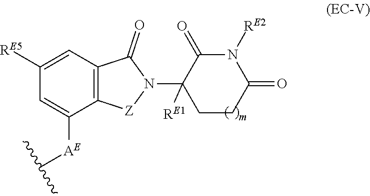

or an enantiomer, a mixture of enantiomers, a diastereomer, a mixture of two or more diastereomers, a tautomer, a mixture of two or more tautomers, or an isotopic variant thereof; wherein A^{E}, R^{E1}, R^{E2}, R^{E5}, Z, and m are each as defined herein. In one embodiment, R^{E5} is hydrogen or fluoro.

In certain embodiments, R^{E} is a moiety having the structure of Formula (EC-VI):

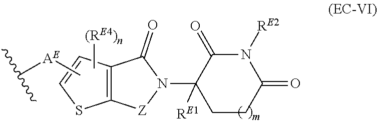

or an enantiomer, a mixture of enantiomers, a diastereomer, a mixture of two or more diastereomers, a tautomer, a mixture of two or more tautomers, or an isotopic variant thereof; wherein n is an integer of 0 or 1; and A^{E}, R^{E1}, R^{E2}, R^{E4}, Z, and m are each as defined herein.

In certain embodiments, R^{E} is a moiety having the structure of Formula (EC-VII):

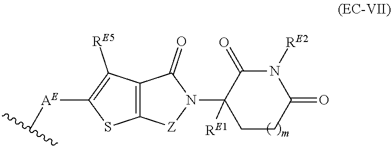

or an enantiomer, a mixture of enantiomers, a diastereomer, a mixture of two or more diastereomers, a tautomer, a mixture of two or more tautomers, or an isotopic variant thereof; wherein A^{E}, R^{E1}, R^{E2}, R^{E5}, Z, and m are each as defined herein. In one embodiment, R^{E5} is hydrogen or fluoro.

In certain embodiments, R^{E} is a moiety having the structure of Formula (EC-VIII):

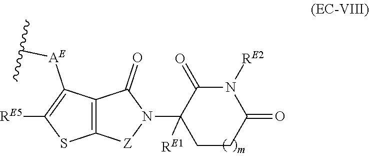

or an enantiomer, a mixture of enantiomers, a diastereomer, a mixture of two or more diastereomers, a tautomer, a mixture of two or more tautomers, or an isotopic variant thereof; wherein A^{E}, R^{E1}, R^{E2}, R^{E5}, Z, and m are each as defined herein. In one embodiment, R^{E5} is hydrogen or fluoro.

In certain embodiments, R^{E} is a moiety having the structure of Formula (EC-IX):

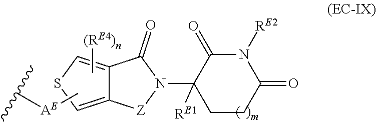

or an enantiomer, a mixture of enantiomers, a diastereomer, a mixture of two or more diastereomers, a tautomer, a mixture of two or more tautomers, or an isotopic variant thereof; wherein n is an integer of 0 or 1; and A^{E}, R^{E1}, R^{E2}, R^{E4}, Z, and m are each as defined herein.

In certain embodiments, R^{E} is a moiety having the structure of Formula (EC-X):

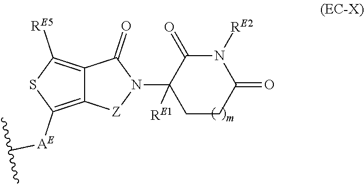

or an enantiomer, a mixture of enantiomers, a diastereomer, a mixture of two or more diastereomers, a tautomer, a mixture of two or more tautomers, or an isotopic variant thereof; wherein A^{E}, R^{E1}, R^{E2}, R^{E5}, Z, and m are each as defined herein. In one embodiment, R^{E5} is hydrogen or fluoro.

In certain embodiments, R^{E} is a moiety having the structure of Formula (EC-XI):

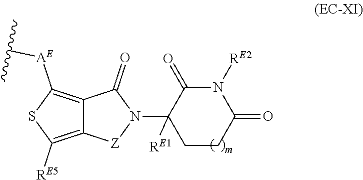

or an enantiomer, a mixture of enantiomers, a diastereomer, a mixture of two or more diastereomers, a tautomer, a mixture of two or more tautomers, or an isotopic variant thereof; wherein A^{E}, R^{E1}, R^{E2}, R^{E5}, Z, and m are each as defined herein. In one embodiment, R^{E5} is hydrogen or fluoro.

In certain embodiments, R^{E} is a moiety having the structure of Formula (EC-XII):

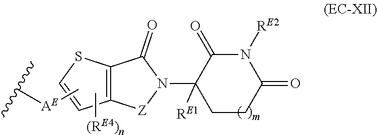

or an enantiomer, a mixture of enantiomers, a diastereomer, a mixture of two or more diastereomers, a tautomer, a mixture of two or more tautomers, or an isotopic variant thereof; wherein n is an integer of 0 or 1; and A^{E}, R^{E1}, R^{E2}, R^{E4}, Z, and m are each as defined herein.

In certain embodiments, R^{E} is a moiety having the structure of Formula (EC-XIII):

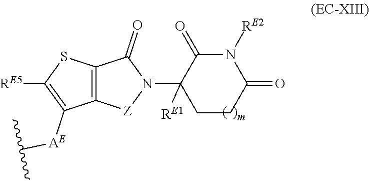

or an enantiomer, a mixture of enantiomers, a diastereomer, a mixture of two or more diastereomers, a tautomer, a mixture of two or more tautomers, or an isotopic variant thereof; wherein A^{E}, R^{E1}, R^{E2}, R^{E5}, Z, and m are each as defined herein. In one embodiment, R^{E5} is hydrogen or fluoro.

In certain embodiments, R^{E} is a moiety having the structure of Formula (EC-XIV):

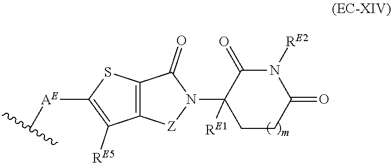

or an enantiomer, a mixture of enantiomers, a diastereomer, a mixture of two or more diastereomers, a tautomer, a mixture of two or more tautomers, or an isotopic variant thereof; wherein A^{E}, R^{E1}, R^{E2}, R^{E5}, Z, and m are each as defined herein. In one embodiment, R^{E5} is hydrogen or fluoro.

In certain embodiments, R^{E} is a moiety having the structure of Formula (EC-XV):

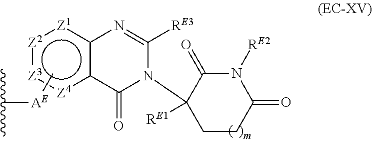

or an enantiomer, a mixture of enantiomers, a diastereomer, a mixture of two or more diastereomers, a tautomer, a mixture of two or more tautomers, or an isotopic variant thereof; wherein R^{E3} is hydrogen, deuterium, halo, or C₁₋₆ alkyl, optionally substituted with one or more, in one embodiment, one, two, three, or four, substituents Q; and A^{E}, R^{E1}, R^{E2}, Z¹, Z², Z³, Z⁴, and m are each as defined herein.

In certain embodiments, R^{E} is a moiety having the structure of Formula (EC-XVI):

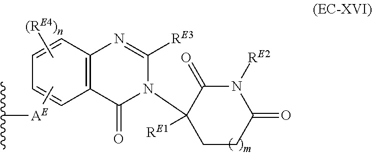

or an enantiomer, a mixture of enantiomers, a diastereomer, a mixture of two or more diastereomers, a tautomer, a mixture of two or more tautomers, or an isotopic variant thereof; wherein n is an integer of 0, 1, 2, or 3; and A^{E}, R^{E1}, R^{E2}, R^{E3}, R^{E4}, and m are each as defined herein.

In certain embodiments, R^{E} is a moiety having the structure of Formula (EC-XVII):

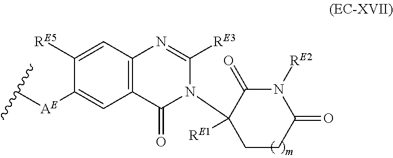

or an enantiomer, a mixture of enantiomers, a diastereomer, a mixture of two or more diastereomers, a tautomer, a mixture of two or more tautomers, or an isotopic variant thereof; wherein A^{E}, R^{E1}, R^{E2}, R^{E3}, R^{E5}, and m are each as defined herein. In one embodiment, R^{E5} is hydrogen or fluoro.

In certain embodiments, R^{E} is a moiety having the structure of Formula (EC-XVIII):

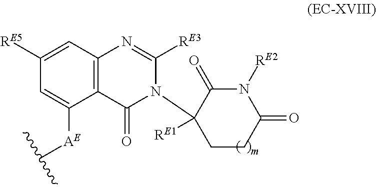

or an enantiomer, a mixture of enantiomers, a diastereomer, a mixture of two or more diastereomers, a tautomer, a mixture of two or more tautomers, or an isotopic variant thereof; wherein A^{E}, R^{E1}, R^{E2}, R^{E3}, R^{E5}, and m are each as defined herein. In one embodiment, R^{E5} is hydrogen or fluoro.

In certain embodiments, R^{E} is a moiety having the structure of Formula (EC-XIX):

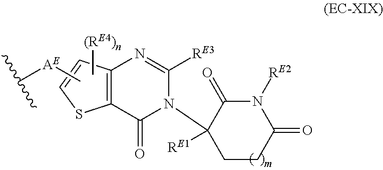

or an enantiomer, a mixture of enantiomers, a diastereomer, a mixture of two or more diastereomers, a tautomer, a mixture of two or more tautomers, or an isotopic variant thereof; wherein n is an integer of 0 or 1; and A^{E}, R^{E1}, R^{E2}, R^{E3}, R^{E4}, and m are each as defined herein.

In certain embodiments, R^{E} is a moiety having the structure of Formula (EC-XX):

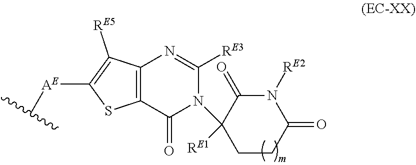

or an enantiomer, a mixture of enantiomers, a diastereomer, a mixture of two or more diastereomers, a tautomer, a mixture of two or more tautomers, or an isotopic variant thereof; wherein A^{E}, R^{E1}, R^{E2}, R^{E3}, R^{E5}, and m are each as defined herein. In one embodiment, R^{E5} is hydrogen or fluoro.

In certain embodiments, R^{E} is a moiety having the structure of Formula (EC-XXI):

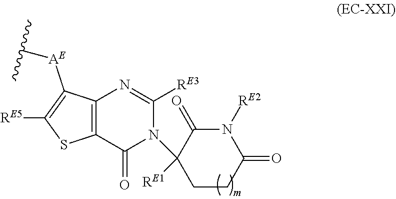

or an enantiomer, a mixture of enantiomers, a diastereomer, a mixture of two or more diastereomers, a tautomer, a mixture of two or more tautomers, or an isotopic variant thereof; wherein A^{E}, R^{E1}, R^{E2}, R^{E3}, R^{E5}, and m are each as defined herein. In one embodiment, R^{E5} is hydrogen or fluoro.

In certain embodiments, R^{E} is a moiety having the structure of Formula (EC-XXII):

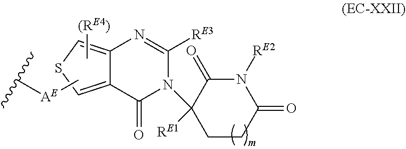

or an enantiomer, a mixture of enantiomers, a diastereomer, a mixture of two or more diastereomers, a tautomer, a mixture of two or more tautomers, or an isotopic variant thereof; wherein n is an integer of 0 or 1; and A^{E}, R^{E1}, R^{E2}, R^{E3}, R^{E4}, and m are each as defined herein.

In certain embodiments, R^{E} is a moiety having the structure of Formula (EC-XXIII):

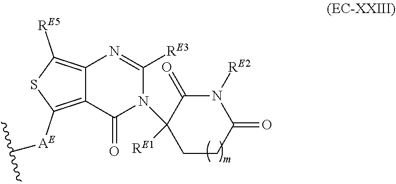

or an enantiomer, a mixture of enantiomers, a diastereomer, a mixture of two or more diastereomers, a tautomer, a mixture of two or more tautomers, or an isotopic variant thereof; wherein A^{E}, R^{E1}, R^{E2}, R^{E3}, R^{E5}, and m are each as defined herein. In one embodiment, R^{E5} is hydrogen or fluoro.

In certain embodiments, R^{E} is a moiety having the structure of Formula (EC-XXIV):

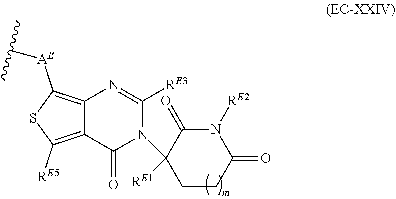

or an enantiomer, a mixture of enantiomers, a diastereomer, a mixture of two or more diastereomers, a tautomer, a mixture of two or more tautomers, or an isotopic variant thereof; wherein A^{E}, R^{E1}, R^{E2}, R^{E3}, R^{E5}, and m are each as defined herein. In one embodiment, R^{E5} is hydrogen or fluoro.

In certain embodiments, R^{E} is a moiety having the structure of Formula (EC-XXV):

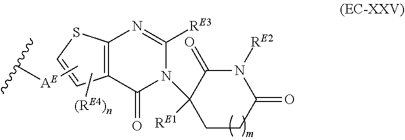

or an enantiomer, a mixture of enantiomers, a diastereomer, a mixture of two or more diastereomers, a tautomer, a mixture of two or more tautomers, or an isotopic variant thereof; wherein n is an integer of 0 or 1; and A^{E}, R^{E1}, R^{E2}, R^{E3}, R^{E4}, and m are each as defined herein.

In certain embodiments, R^{E} is a moiety having the structure of Formula (EC-XXVI):

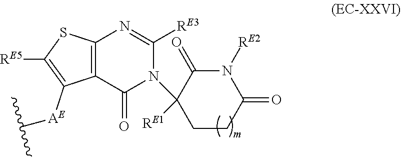

or an enantiomer, a mixture of enantiomers, a diastereomer, a mixture of two or more diastereomers, a tautomer, a mixture of two or more tautomers, or an isotopic variant thereof; wherein A^{E}, R^{E1}, R^{E2}, R^{E3}, R^{E5}, and m are each as defined herein. In one embodiment, R^{E5} is hydrogen or fluoro.

In certain embodiments, R^{E} is a moiety having the structure of Formula (EC-XXVII):

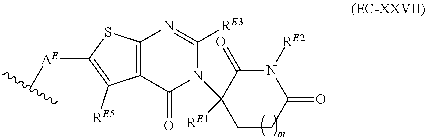

or an enantiomer, a mixture of enantiomers, a diastereomer, a mixture of two or more diastereomers, a tautomer, a mixture of two or more tautomers, or an isotopic variant thereof; wherein A^{E}, R^{E1}, R^{E2}, R^{E3}, R^{E5}, and m are each as defined herein. In one embodiment, R^{E5} is hydrogen or fluoro.

In certain embodiments, R^{E} is a moiety having the structure of Formula (EC-XXVIII):

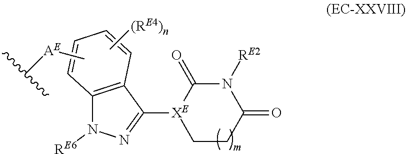

or an enantiomer, a mixture of enantiomers, a diastereomer, a mixture of two or more diastereomers, a tautomer, a mixture of two or more tautomers, or an isotopic variant thereof; wherein:
R^{E6} is (i) hydrogen; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, or three, substituents Q; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c};
X^{E} is C(R^{E1}) or N; and
A^{E}, R^{E1}, R^{E2}, R^{E4}, m, and n are each as defined herein.

In certain embodiments, R^{E} is a moiety having the structure of Formula (EC-XXIX):

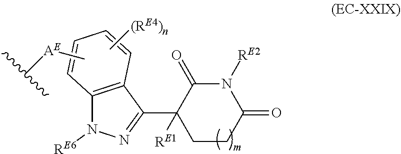

or an enantiomer, a mixture of enantiomers, a diastereomer, a mixture of two or more diastereomers, a tautomer, a mixture of two or more tautomers, or an isotopic variant thereof; wherein A^{E}, R^{E1}, R^{E2}, R^{E4}, R^{E6}, m, and n are each as defined herein.

In certain embodiments, R^{E} is a moiety having the structure of Formula (EC-XXX):

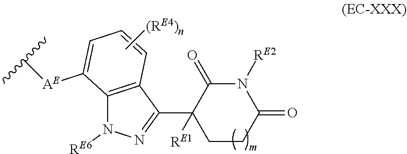

or an enantiomer, a mixture of enantiomers, a diastereomer, a mixture of two or more diastereomers, a tautomer, a mixture of two or more tautomers, or an isotopic variant thereof; wherein A^{E}, R^{E1}, R^{E2}, R^{E4}, R^{E6}, m, and n are each as defined herein.

In certain embodiments, R^{E} is a moiety having the structure of Formula (EC-XXXI):

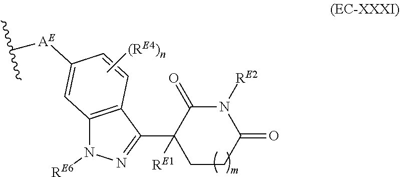

or an enantiomer, a mixture of enantiomers, a diastereomer, a mixture of two or more diastereomers, a tautomer, a mixture of two or more tautomers, or an isotopic variant thereof, wherein A^{E}, R^{E1}, R^{E2}, R^{E4}, R^{E6}, m, and n are each as defined herein.

In certain embodiments, R^{E} is a moiety having the structure of Formula (EC-XXXII):

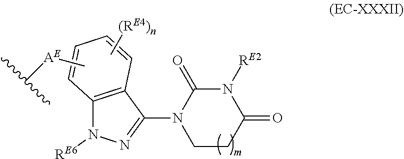

or an enantiomer, a mixture of enantiomers, a diastereomer, a mixture of two or more diastereomers, a tautomer, a mixture of two or more tautomers, or an isotopic variant thereof; wherein A^{E}, R^{E2}, R^{E4}, R^{E6}, m, and n are each as defined herein.

In certain embodiments, R^{E} is a moiety having the structure of Formula (EC-XXXV):

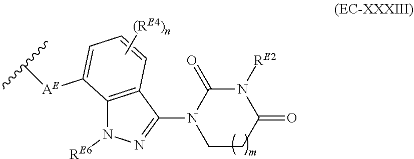

or an enantiomer, a mixture of enantiomers, a diastereomer, a mixture of two or more diastereomers, a tautomer, a mixture of two or more tautomers, or an isotopic variant thereof; wherein A^{E}, R^{E2}, R^{E4}, R^{E6}, m, and n are each as defined herein.

In certain embodiments, R^{E} is a moiety having the structure of Formula (EC-XXXIV):

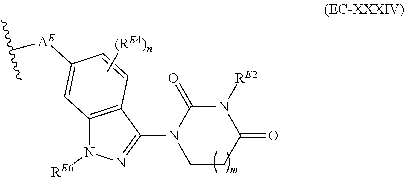

or an enantiomer, a mixture of enantiomers, a diastereomer, a mixture of two or more diastereomers, a tautomer, a mixture of two or more tautomers, or an isotopic variant thereof; wherein A^{E}, R^{E2}, R^{E4}, R^{E6}, m, and n are each as defined herein.

In certain embodiments, R^{E} is a moiety having the structure of Formula (EC-XXXV):

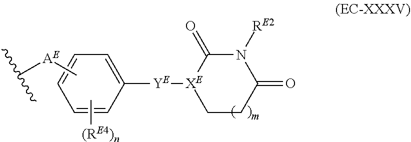

or an enantiomer, a mixture of enantiomers, a diastereomer, a mixture of two or more diastereomers, a tautomer, a mixture of two or more tautomers, or an isotopic variant thereof; wherein:
Y^{E} is a bond, C₁₋₆ alkylene, —O—, —S—, —S(O)—, —S(O₂)—, or —N(R^{E7})—;
R^{E7} is hydrogen or C₁₋₆ alkyl; and
A^{E}, R^{E2}, R^{E4}, X^{E}, m, and n are each as defined herein.

In certain embodiments, R^{E} is a moiety having the structure of Formula (EC-XXXVI):

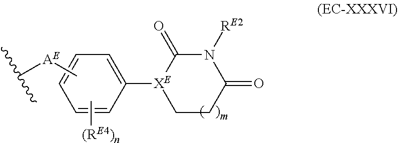

or an enantiomer, a mixture of enantiomers, a diastereomer, a mixture of two or more diastereomers, a tautomer, a mixture of two or more tautomers, or an isotopic variant thereof; wherein A^{E}, R^{E2}, R^{E4}, X^{E}, m, and n are each as defined herein.

In certain embodiments, R^{E} is a moiety having the structure of Formula (EC-XXXVII):

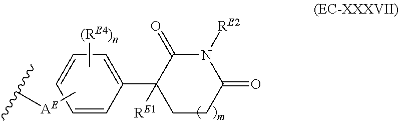

or an enantiomer, a mixture of enantiomers, a diastereomer, a mixture of two or more diastereomers, a tautomer, a mixture of two or more tautomers, or an isotopic variant thereof; wherein A^{E}, R^{E1}, R^{E2}, R^{E4}, m, and n are each as defined herein.

In certain embodiments, R^{E} is a moiety having the structure of Formula (EC-XXXVIII):

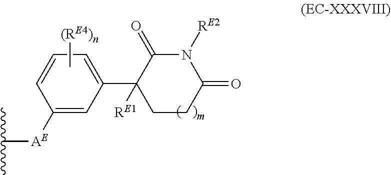

or an enantiomer, a mixture of enantiomers, a diastereomer, a mixture of two or more diastereomers, a tautomer, a mixture of two or more tautomers, or an isotopic variant thereof; wherein A^{E}, R^{E1}, R^{E2}, R^{E4}, m, and n are each as defined herein.

In certain embodiments, R^{E} is a moiety having the structure of Formula (EC-XXXIX):

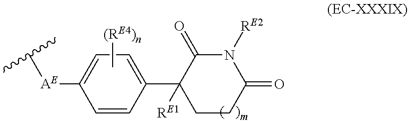

or an enantiomer, a mixture of enantiomers, a diastereomer, a mixture of two or more diastereomers, a tautomer, a mixture of two or more tautomers, or an isotopic variant thereof; wherein A^{E}, R^{E1}, R^{E2}, R^{E4}, m, and n are each as defined herein.

In certain embodiments, R^{E} is a moiety having the structure of Formula (EC-XL):

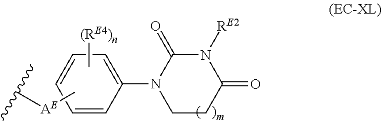

or an enantiomer, a mixture of enantiomers, a diastereomer, a mixture of two or more diastereomers, a tautomer, a mixture of two or more tautomers, or an isotopic variant thereof; wherein A^{E}, R^{E2}, R^{E4}, m, and n are each as defined herein.

In certain embodiments, R^{E} is a moiety having the structure of Formula (EC-XLI):

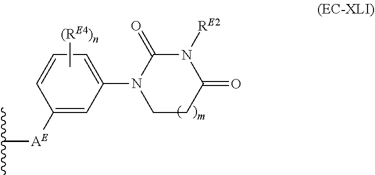

or an enantiomer, a mixture of enantiomers, a diastereomer, a mixture of two or more diastereomers, a tautomer, a mixture of two or more tautomers, or an isotopic variant thereof; wherein A^{E}, R^{E2}, R^{E4}, m, and n are each as defined herein.

In certain embodiments, R^{E} is a moiety having the structure of Formula (EC-XLII):

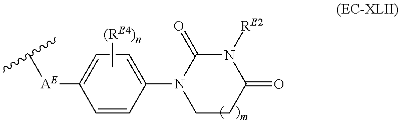

or an enantiomer, a mixture of enantiomers, a diastereomer, a mixture of two or more diastereomers, a tautomer, a mixture of two or more tautomers, or an isotopic variant thereof; wherein A^{E}, R^{E2}, R^{E4}, m, and n are each as defined herein.

In certain embodiments, R^{E} is a moiety having the structure of Formula (EC-XLIII):

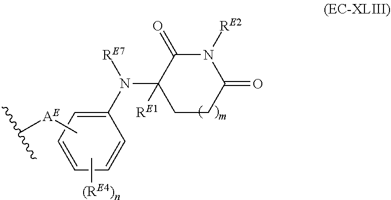

or an enantiomer, a mixture of enantiomers, a diastereomer, a mixture of two or more diastereomers, a tautomer, a mixture of two or more tautomers, or an isotopic variant thereof; wherein A^{E}, R^{E1}, R^{E2}, R^{E4}, R^{E7}, m, and n are each as defined herein.

In certain embodiments, R^{E} is a moiety having the structure of Formula (EC-XLIV):

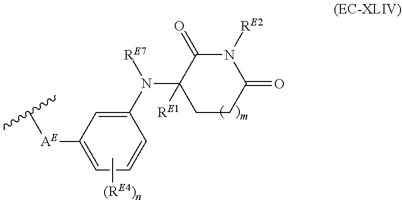

or an enantiomer, a mixture of enantiomers, a diastereomer, a mixture of two or more diastereomers, a tautomer, a mixture of two or more tautomers, or an isotopic variant thereof; wherein A^{E}, R^{E1}, R^{E2}, R^{E4}, R^{E7}, m, and n are each as defined herein.

In certain embodiments, R^{E} is a moiety having the structure of Formula (EC-XLV):

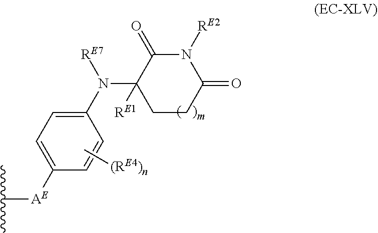

or an enantiomer, a mixture of enantiomers, a diastereomer, a mixture of two or more diastereomers, a tautomer, a mixture of two or more tautomers, or an isotopic variant thereof; wherein A^{E}, R^{E1}, R^{E2}, R^{E4}, R^{E7}, m, and n are each as defined herein.

In certain embodiments, R^{E} is a moiety of an E3 ubiquitin ligase binder disclosed in U.S. Pat. No. 9,938,302 B2; U.S. Pat. No. 10,336,771 B2; U.S. Pat. No. 10,406,165 B2; U.S. Pat. No. 10,513,515 B2; US 2019/0322682 A1; US 2020/0000814 A1; US 2020/0148663 A1; US 2020/0369679 A1; and WO 2019/173224 A1; the disclosure of each of which is incorporated herein by reference in its entirety. In certain embodiments, R^{E} is a moiety of an E3 ubiquitin ligase binder disclosed in U.S. Pat. No. 9,938,302 B2, in one embodiment, one of compounds 1 to 57 disclosed therein in cols. 108 to 137, which are incorporated herein by reference in their entireties. In certain embodiments, R^{E} is a moiety of an E3 ubiquitin ligase binder disclosed in U.S. Pat. No. 10,336,771 B2, in one embodiment, one of compounds 1 to 57 and 64 to 66 disclosed therein in cols. 113 to 161 and 169 to 172, which are incorporated herein by reference in their entireties. In certain embodiments, R^{E} is a moiety of an E3 ubiquitin ligase binder disclosed in U.S. Pat. No. 10,406,165 B2, in one embodiment, one of compounds 1 to 27 disclosed therein in cols. 40 to 64, which are incorporated herein by reference in their entireties. In certain embodiments, R^{E} is a moiety of an E3 ubiquitin ligase binder disclosed in U.S. Pat. No. 10,513,515 B2, in one embodiment, one of compounds 1 to 5, 7 to 12, 14 to 16, 19, 23, and 27 disclosed therein in cols. 97 to 104, 106 to 112, 114 to 117, 122, 126, and 132, which are incorporated herein by reference in their entireties. In certain embodiments, R^{E} is a moiety of an E3 ubiquitin ligase binder disclosed in US 2019/0322682 A1, in one embodiment, one of compounds 1 to 15 disclosed therein on pages 31 to 36, which are incorporated herein by reference in their entireties. In certain embodiments, R^{E} is a moiety of an E3 ubiquitin ligase binder disclosed in US 2020/0000814 A1, in one embodiment, one of compounds 1 to 21 disclosed therein on pages 26 to 41, which are incorporated herein by reference in their entireties. In certain embodiments, R^{E} is a moiety of an E3 ubiquitin ligase binder disclosed in US 2020/0148663 A1, in one embodiment, one of compounds 1 to 21 disclosed therein on pages 18 to 34, which are incorporated herein by reference in their entireties. In certain embodiments, R^{E} is a moiety of an E3 ubiquitin ligase binder disclosed in WO 2019/173224 A1, in one embodiment, one of compounds 1 to 3 disclosed therein on pages 62 to 65 and the compounds disclosed therein on page 78, which are incorporated herein by reference in their entireties. In certain embodiments, R^{E} is a moiety of an E3 ubiquitin ligase binder disclosed in US 2020/0369679 A1, in one embodiment, one of compounds I-1 to I-106 and II-1 to II-164 disclosed therein on pages 50 to 101, which are incorporated herein by reference in their entireties.

In certain embodiments, R^{E} is a moiety of an E3 ubiquitin ligase binder disclosed in US 2020/0199073 A1, the disclosure of which is incorporated herein by reference in its entirety. In certain embodiments, R^{E} is a moiety of an E3 ubiquitin ligase binder disclosed in US Application No. US 2020/0199073 A1, in one embodiment, one of compounds 1 to 291 disclosed therein on pages 118 to 193, which are incorporated herein by reference in their entireties.

In certain embodiments, in any one of Formulae (EC-I) to (EC-XXVII),
A^{E} is a bond, —O—, —N(R^{1b})—, C₂₋₆ alkynylene, heterocyclylene, C₁₋₆ heteroalkylene-C₆₋₁₄ arylene, or C₂₋₆ alkynylene-heterocyclylene, where each heteroalkylene, alkynylene, arylene, and heterocyclylene is optionally substituted with one or more, in one embodiment, one, two, three, or four, substituents Q;
Z, if present, is —CH₂— or —C(O)—;
m is an integer of 0, 1, or 2;
R^{E1} and R^{E2} are each hydrogen; and
R^{E5}, if present, is independently hydrogen or fluoro.

In certain embodiments, in any one of Formulae (EC-I) to (EC-XXVII),
A^{E} is a bond, —O—, —NH—, ethynediyl, piperidindiyl, piperazindiyl, (phendiyl)oxymethanediyl, or (piperidindiyl)ethynediyl;
Z, if present, is —CH₂— or —C(O)—;
m is an integer of 0, 1, or 2;
R^{E1} and R^{E2} are each hydrogen; and
R^{E5}, if present, is independently hydrogen or fluoro.

In certain embodiments, in any one of Formulae (EC-I) to (EC-XXVII),
A^{E} is a bond, —NH—, piperidin-1,3-diyl, piperidin-1,4-diyl, piperaz-1,4-diyl, (phen-1,4-diyl)oxymethanediyl, or (piperidin-1,4-diyl)ethynediyl;
Z, if present, is —CH₂— or —C(O)—;
m is an integer of 0, 1, or 2;
R^{E1} and R^{E2} are each hydrogen; and
R^{E5}, if present, is independently hydrogen or fluoro.

In one embodiment, in a compound of any one of Formulae (I) to (XVI), R^{E} is a moiety having the structure of Formula (EC-I), or an enantiomer, a mixture of enantiomers, a diastereomer, a mixture of two or more diastereomers, a tautomer, a mixture of two or more tautomers, or an isotopic variant thereof. In another embodiment, in a compound of any one of Formulae (I) to (XVI), R^{E} is a moiety having the structure of Formula (EC-II), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-III), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-IV), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-V), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-VI), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-VII), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-VIII), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-IX), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-X), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-XI), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-XII), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-XIII), or an enantiomer, a mixture of enantiomers, a diastereomer, a mixture of two or more diastereomers, a tautomer, a mixture of two or more tautomers, or an isotopic variant thereof. In still another embodiment, in a compound of any one of Formulae (I) to (XVI), R^{E} is a moiety having the structure of Formula (EC-XIV), or an enantiomer, a mixture of enantiomers, a diastereomer, a mixture of two or more diastereomers, a tautomer, a mixture of two or more tautomers, or an isotopic variant thereof.

In one embodiment, in a compound of any one of Formulae (I) to (XVI), R^{E} is a moiety having the structure of Formula (EC-XV), or an enantiomer, a mixture of enantiomers, a diastereomer, a mixture of two or more diastereomers, a tautomer, a mixture of two or more tautomers, or an isotopic variant thereof. In another embodiment, in a compound of any one of Formulae (I) to (XVI), R^{E} is a moiety having the structure of Formula (EC-XVI), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-XVII), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-XVIII), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-XIX), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-XX), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-XXI), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-XXII), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-XXIII), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-XXIV), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-XXV), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-XXVI), or an enantiomer, a mixture of enantiomers, a diastereomer, a mixture of two or more diastereomers, a tautomer, a mixture of two or more tautomers, or an isotopic variant thereof. In still another embodiment, in a compound of any one of Formulae (I) to (XVI), R^{E} is a moiety having the structure of Formula (EC-XXVII), or an enantiomer, a mixture of enantiomers, a diastereomer, a mixture of two or more diastereomers, a tautomer, a mixture of two or more tautomers, or an isotopic variant thereof.

In one embodiment, in a compound of any one of Formulae (I) to (XVI), R^{E} is a moiety having the structure of Formula (EC-XXVIII), or an enantiomer, a mixture of enantiomers, a diastereomer, a mixture of two or more diastereomers, a tautomer, a mixture of two or more tautomers, or an isotopic variant thereof. In another embodiment, in a compound of any one of Formulae (I) to (XVI), R^{E} is a moiety having the structure of Formula (EC-XXIX), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-XXX), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-XXXI), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-XXXII), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-XXXIII), or an enantiomer, a mixture of enantiomers, a diastereomer, a mixture of two or more diastereomers, a tautomer, a mixture of two or more tautomers, or an isotopic variant thereof. In still another embodiment, in a compound of any one of Formulae (I) to (XVI), R^{E} is a moiety having the structure of Formula (EC-XXXIV), or an enantiomer, a mixture of enantiomers, a diastereomer, a mixture of two or more diastereomers, a tautomer, a mixture of two or more tautomers, or an isotopic variant thereof.

In one embodiment, in a compound of any one of Formulae (I) to (XVI), R^{E} is a moiety having the structure of Formula (EC-XXXV), or an enantiomer, a mixture of enantiomers, a diastereomer, a mixture of two or more diastereomers, a tautomer, a mixture of two or more tautomers, or an isotopic variant thereof. In another embodiment, in a compound of any one of Formulae (I) to (XVI), R^{E} is a moiety having the structure of Formula (EC-XXXVI), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-XXXVII), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-XXXVIII), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-XXXIX), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-XL), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-XLI), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-XLII), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-XLIII), or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EC-XLIV), or an enantiomer, a mixture of enantiomers, a diastereomer, a mixture of two or more diastereomers, a tautomer, a mixture of two or more tautomers, or an isotopic variant thereof. In still another embodiment, in a compound of any one of Formulae (I) to (XVI), R^{E} is a moiety having the structure of Formula (EC-XLV), or an enantiomer, a mixture of enantiomers, a diastereomer, a mixture of two or more diastereomers, a tautomer, a mixture of two or more tautomers, or an isotopic variant thereof.

In certain embodiments, R^{E} is a moiety having the structure of:

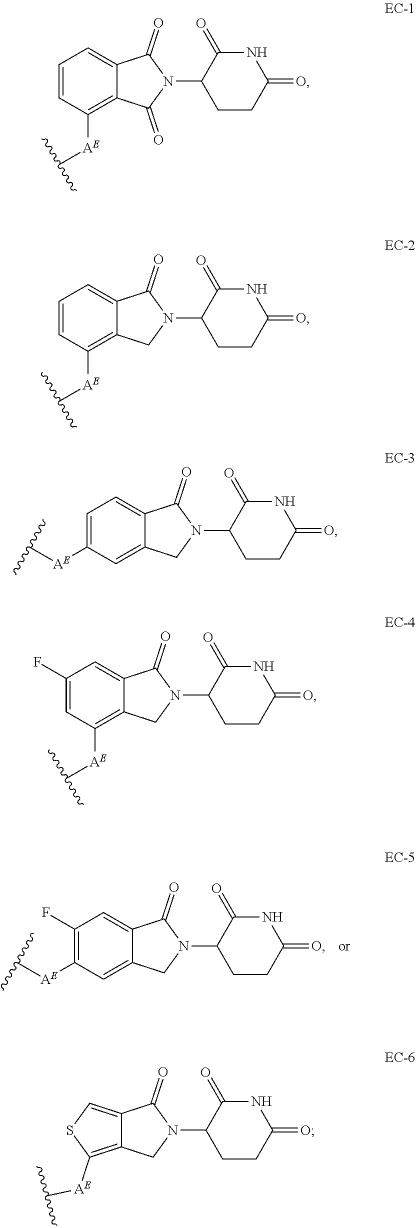

or an enantiomer, a mixture of enantiomers, a diastereomer, a mixture of two or more diastereomers, a tautomer, a mixture of two or more tautomers, or an isotopic variant thereof; wherein A^{E} is as defined herein.

In one embodiment, in any one of Formulae EC-1 to EC-6, A^{E} is a bond, —O—, —NH—, ethynediyl, piperidindiyl, piperazindiyl, (phendiyl)oxymethanediyl, or (piperidindiyl)-ethynediyl. In another embodiment, in any one of Formulae EC-1 to EC-6, A^{E} is a bond. In yet another embodiment, in any one of Formulae EC-1 to EC-6, A^{E} is —O—. In yet another embodiment, in any one of Formulae EC-1 to EC-6, A^{E} is —NH—. In yet another embodiment, in any one of Formulae EC-1 to EC-6, A^{E} is ethynediyl. In yet another embodiment, in any one of Formulae EC-1 to EC-6, A^{E} is piperidindiyl. In yet another embodiment, in any one of Formulae EC-1 to EC-6, A^{E} is piperazindiyl. In yet another embodiment, in any one of Formulae EC-1 to EC-6, A^{E} is (phendiyl)oxymethanediyl. In still another embodiment, in any one of Formulae EC-1 to EC-6, A^{E} is (piperidindiyl)ethynyl.

In one embodiment, in any one of Formulae EC-1 to EC-6, A^{E} is a bond, —O—, —NH—, ethynediyl, piperidin-1,3-diyl, piperidin-1,4-diyl, piperazin-1,4-diyl, (phen-1,4-diyl)-oxymethanediyl, or (piperidin-1,4-diyl)ethynediyl. In another embodiment, in any one of Formulae EC-1 to EC-6, A^{E} is piperidin-1,3-diyl. In yet another embodiment, in any one of Formulae EC-1 to EC-6, A^{E} is piperidin-1,4-diyl. In yet another embodiment, in any one of Formulae EC-1 to EC-6, A^{E} is piperazin-1,4-diyl. In yet another embodiment, in any one of Formulae EC-1 to EC-6, A^{E} is (phen-1,4-diyl)oxymethanediyl. In still another embodiment, in any one of Formulae EC-1 to EC-6, A^{E} is (piperidin-1,4-diyl)ethynediyl.

In one embodiment, in a compound of any one of Formulae (I) to (XVI), R^{E} is a moiety having the structure of Formula EC-1, or an enantiomer, a mixture of enantiomers, a diastereomer, a mixture of two or more diastereomers, a tautomer, a mixture of two or more tautomers, or an isotopic variant thereof. In another embodiment, in a compound of any one of Formulae (I) to (XVI), R^{E} is a moiety having the structure of Formula EC-2, or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula EC-3, or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula EC-4, or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula EC-5, or an enantiomer, a mixture of enantiomers, a diastereomer, a mixture of two or more diastereomers, a tautomer, a mixture of two or more tautomers, or an isotopic variant thereof. In still another embodiment, in a compound of any one of Formulae (I) to (XVI), R^{E} is a moiety having the structure of Formula EC-6, or an enantiomer, a mixture of enantiomers, a diastereomer, a mixture of two or more diastereomers, a tautomer, a mixture of two or more tautomers, or an isotopic variant thereof.

In certain embodiments, R^{E} is a moiety having the structure of:

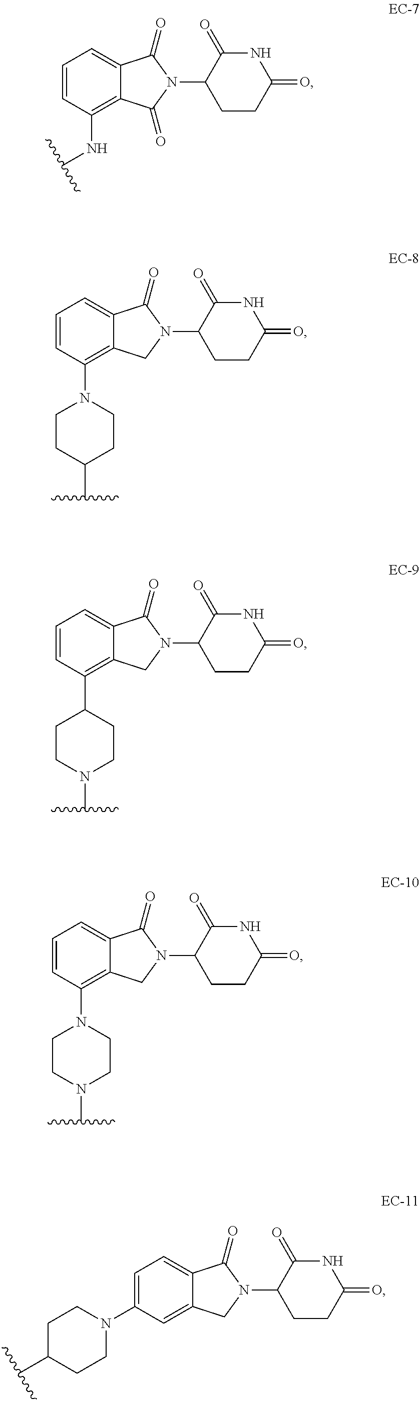 [SMILES: *Nc1cccc2c1C(=O)N(C1CCC(=O)NC1=O)C2=O ; *C1CCN(c2cccc3c2CN(C2CCC(=O)NC2=O)C3=O)CC1 ; *N1CCC(c2cccc3c2CN(C2CCC(=O)NC2=O)C3=O)CC1 ; *N1CCN(c2cccc3c2CN(C2CCC(=O)NC2=O)C3=O)CC1 ; *C1CCN(c2ccc3c(c2)CN(C2CCC(=O)NC2=O)C3=O)CC1]

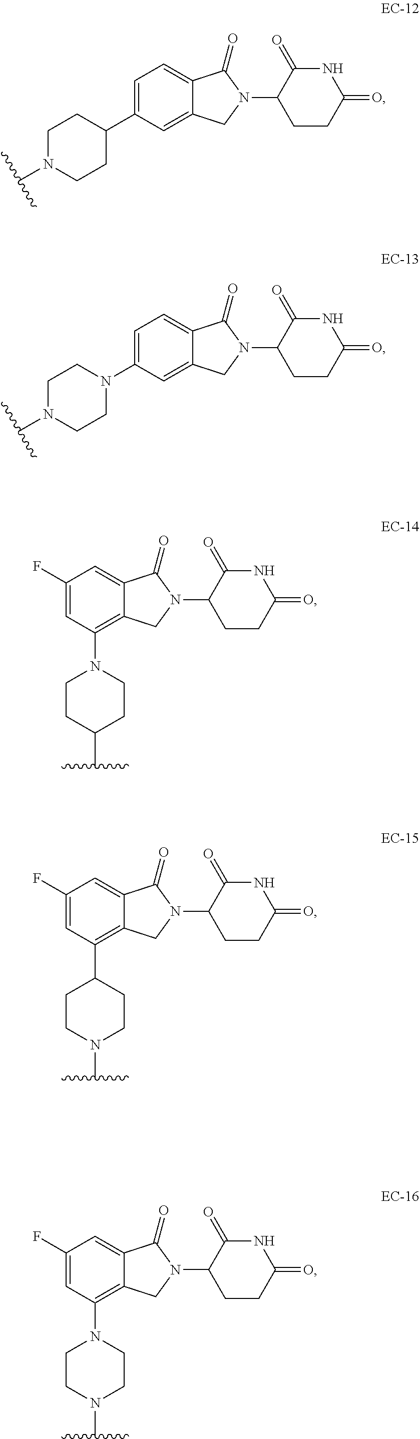 [SMILES: *N1CCC(c2ccc3c(c2)CN(C2CCC(=O)NC2=O)C3=O)CC1 ; *N1CCN(c2ccc3c(c2)CN(C2CCC(=O)NC2=O)C3=O)CC1 ; *C1CCN(c2cc(F)cc3c2CN(C2CCC(=O)NC2=O)C3=O)CC1 ; *N1CCC(c2cc(F)cc3c2CN(C2CCC(=O)NC2=O)C3=O)CC1 ; *N1CCN(c2cc(F)cc3c2CN(C2CCC(=O)NC2=O)C3=O)CC1]

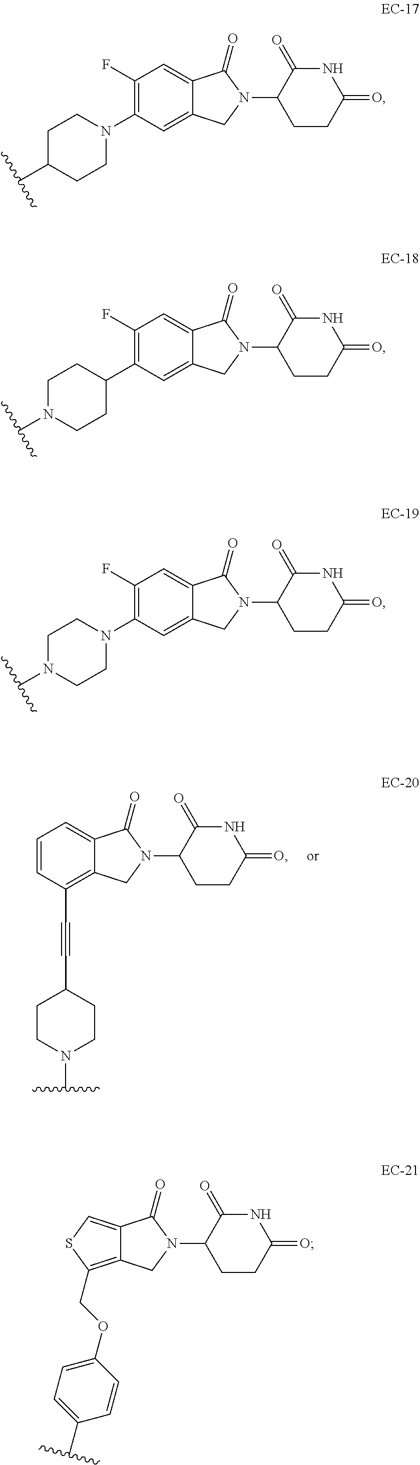 [SMILES: *C1CCN(c2cc3c(cc2F)C(=O)N(C2CCC(=O)NC2=O)C3)CC1 ; *N1CCC(c2cc3c(cc2F)C(=O)N(C2CCC(=O)NC2=O)C3)CC1 ; *N1CCN(c2cc3c(cc2F)C(=O)N(C2CCC(=O)NC2=O)C3)CC1 ; *N1CCC(C#Cc2cccc3c2CN(C2CCC(=O)NC2=O)C3=O)CC1 ; "or" ; *c1ccc(OCc2scc3c2CN(C2CCC(=O)NC2=O)C3=O)cc1]

or an enantiomer, a mixture of enantiomers, a tautomer, a mixture of two or more tautomers, or an isotopic variant thereof.

In one embodiment, in a compound of any one of Formulae (I) to (XVI), R^{E} is a moiety having the structure of Formula EC-7, or an enantiomer, a mixture of enantiomers, a tautomer, a mixture of two or more tautomers, or an isotopic variant thereof. In another embodiment, in a compound of any one of Formulae (I) to (XVI), R^{E} is a moiety having the structure of Formula EC-8, or an enantiomer, a mixture of enantiomers, a tautomer, a mixture of two or more tautomers, or an isotopic variant thereof. In yet another embodiment, in a compound of any one of Formulae (I) to (XVI), R^{E} is a moiety having the structure of Formula EC-9, or an enantiomer, a mixture of enantiomers, a tautomer, a mixture of two or more tautomers, or an isotopic variant thereof. In yet another embodiment, in a compound of any one of Formulae (I) to (XVI), R^{E} is a moiety having the structure of Formula EC-10, or an enantiomer, a mixture of enantiomers, a tautomer, a mixture of two or more tautomers, or an isotopic variant thereof. In yet another embodiment, in a compound of any one of Formulae (I) to (XVI), R^{E} is a moiety having the structure of Formula EC-11, or an enantiomer, a mixture of enantiomers, a tautomer, a mixture of two or more tautomers, or an isotopic variant thereof. In yet another embodiment, in a compound of any one of Formulae (I) to (XVI), R^{E} is a moiety having the structure of Formula EC-12, or an enantiomer, a mixture of enantiomers, a tautomer, a mixture of two or more tautomers, or an isotopic variant thereof. In yet another embodiment, in a compound of any one of Formulae (I) to (XVI), R^{E} is a moiety having the structure of Formula EC-13, or an enantiomer, a mixture of enantiomers, a tautomer, a mixture of two or more tautomers, or an isotopic variant thereof. In yet another embodiment, in a compound of any one of Formulae (I) to (XVI), R^{E} is a moiety having the structure of Formula EC-14, or an enantiomer, a mixture of enantiomers, a tautomer, a mixture of two or more tautomers, or an isotopic variant thereof. In yet another embodiment, in a compound of any one of Formulae (I) to (XVI), R^{E} is a moiety having the structure of Formula EC-15, or an enantiomer, a mixture of enantiomers, a tautomer, a mixture of two or more tautomers, or an isotopic variant thereof. In yet another embodiment, in a compound of any one of Formulae (I) to (XVI), R^{E} is a moiety having the structure of Formula EC-16, or an enantiomer, a mixture of enantiomers, a tautomer, a mixture of two or more tautomers, or an isotopic variant thereof. In yet another embodiment, in a compound of any one of Formulae (I) to (XVI), R^{E} is a moiety having the structure of Formula EC-17, or an enantiomer, a mixture of enantiomers, a tautomer, a mixture of two or more tautomers, or an isotopic variant thereof. In yet another embodiment, in a compound of any one of Formulae (I) to (XVI), R^{E} is a moiety having the structure of Formula EC-18, or an enantiomer, a mixture of enantiomers, a tautomer, a mixture of two or more tautomers, or an isotopic variant thereof. In yet another embodiment, in a compound of any one of Formulae (I) to (XVI), R^{E} is a moiety having the structure of Formula EC-19, or an enantiomer, a mixture of enantiomers, a tautomer, a mixture of two or more tautomers, or an isotopic variant thereof. In yet another embodiment, in a compound of any one of Formulae (I) to (XVI), R^{E} is a moiety having the structure of Formula EC-20, or an enantiomer, a mixture of enantiomers, a tautomer, a mixture of two or more tautomers, or an isotopic variant thereof. In still another embodiment, in a compound of any one of Formulae (I) to (XVI), R^{E} is a moiety having the structure of Formula EC-21, or an enantiomer, a mixture of enantiomers, a tautomer, a mixture of two or more tautomers, or an isotopic variant thereof.

In certain embodiments, R^{E} is a moiety having the structure of.

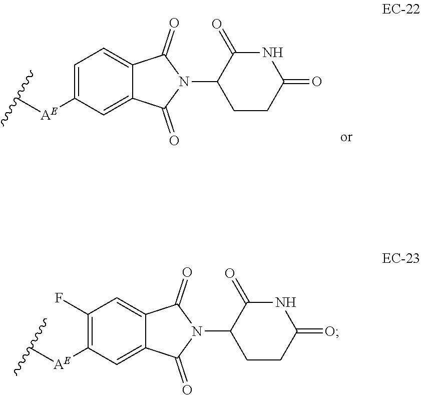

or an enantiomer, a mixture of enantiomers, a diastereomer, a mixture of two or more diastereomers, a tautomer, a mixture of two or more tautomers, or an isotopic variant thereof; wherein A^{E} is as defined herein.

In one embodiment, in Formula EC-22 or EC-23, A^{E} is a bond, —O—, —NH—, ethynediyl, piperidindiyl, piperazindiyl, (phendiyl)oxymethanediyl, or (piperidindiyl)ethynediyl. In another embodiment, in Formula EC-22 or EC-23, A^{E} is a bond. In yet another embodiment, in Formula EC-22 or EC-23, A^{E} is —O—. In yet another embodiment, in Formula EC-22 or EC-23, A^{E} is —NH—. In yet another embodiment, in Formula EC-22 or EC-23, A^{E} is ethynediyl. In yet another embodiment, in Formula EC-22 or EC-23, A^{E} is piperidindiyl. In yet another embodiment, in Formula EC-22 or EC-23, A^{E} is piperazindiyl. In yet another embodiment, in Formula EC-22 or EC-23, A^{E} is (phendiyl)oxymethanediyl. In still another embodiment, in Formula EC-22 or EC-23, A^{E} is (piperidindiyl)ethynyl.

In one embodiment, in Formula EC-22 or EC-23, A^{E} is a bond, —O—, —NH—, ethynediyl, piperidin-1,3-diyl, piperidin-1,4-diyl, piperazin-1,4-diyl, (phen-1,4-diyl)oxymethanediyl, or (piperidin-1,4-diyl)ethynediyl. In another embodiment, in Formula EC-22 or EC-23, A^{E} is piperidin-1,3-diyl. In yet another embodiment, in Formula EC-22 or EC-23, A^{E} is piperidin-1,4-diyl. In yet another embodiment, in Formula EC-22 or EC-23, A^{E} is piperazin-1,4-diyl. In yet another embodiment, in Formula EC-22 or EC-23, A^{E} is (phen-1,4-diyl)oxymethanediyl. In still another embodiment, in Formula EC-22 or EC-23, A^{E} is (piperidin-1,4-diyl)ethynediyl.

In one embodiment, in a compound of any one of Formulae (I) to (XVI), R^{E} is a moiety having the structure of Formula EC-22, or an enantiomer, a mixture of enantiomers, a diastereomer, a mixture of two or more diastereomers, a tautomer, a mixture of two or more tautomers, or an isotopic variant thereof. In another embodiment, in a compound of any one of Formulae (I) to (XVI), R^{E} is a moiety having the structure of Formula EC-23, or an enantiomer, a mixture of enantiomers, a diastereomer, a mixture of two or more diastereomers, a tautomer, a mixture of two or more tautomers, or an isotopic variant thereof.

In certain embodiments, R^{E} is a moiety having the structure of:

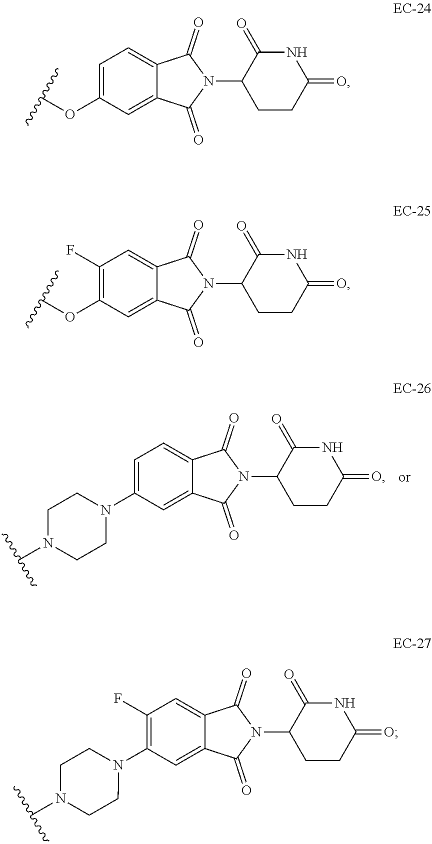 [SMILES: *Oc1ccc2c(c1)C(=O)N(C1CCC(=O)NC1=O)C2=O ; *Oc1cc2c(cc1F)C(=O)N(C1CCC(=O)NC1=O)C2=O ; *N1CCN(c2ccc3c(c2)C(=O)N(C2CCC(=O)NC2=O)C3=O)CC1 ; *N1CCN(c2cc3c(cc2F)C(=O)N(C2CCC(=O)NC2=O)C3=O)CC1]

or an enantiomer, a mixture of enantiomers, a tautomer, a mixture of two or more tautomers, or an isotopic variant thereof.

In one embodiment, in a compound of any one of Formulae (I) to (XVI), R^{E} is a moiety having the structure of Formula EC-24, or an enantiomer, a mixture of enantiomers, a diastereomer, a mixture of two or more diastereomers, a tautomer, a mixture of two or more tautomers, or an isotopic variant thereof. In another embodiment, in a compound of any one of Formulae (I) to (XVI), R^{E} is a moiety having the structure of Formula EC-25, or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula EC-26, or an enantiomer, a mixture of enantiomers, a diastereomer, a mixture of two or more diastereomers, a tautomer, a mixture of two or more tautomers, or an isotopic variant thereof. In still another embodiment, in a compound of any one of Formulae (I) to (XVI), R^{E} is a moiety having the structure of Formula EC-27, or an enantiomer, a mixture of enantiomers, a diastereomer, a mixture of two or more diastereomers, a tautomer, a mixture of two or more tautomers, or an isotopic variant thereof.

In certain embodiments, R^{E} is a moiety having the structure of.

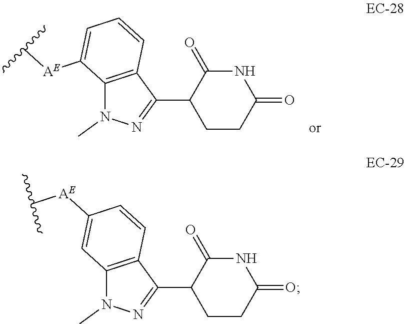

or an enantiomer, a mixture of enantiomers, a tautomer, a mixture of two or more tautomers, or an isotopic variant thereof; wherein A^{E} is as defined herein.

In one embodiment, in a compound of any one of Formulae (I) to (XVI), R^{E} is a moiety having the structure of Formula EC-28, or an enantiomer, a mixture of enantiomers, a diastereomer, a mixture of two or more diastereomers, a tautomer, a mixture of two or more tautomers, or an isotopic variant thereof. In another embodiment, in a compound of any one of Formulae (I) to (XVI), R^{E} is a moiety having the structure of Formula EC-29, or an enantiomer, a mixture of enantiomers, a diastereomer, a mixture of two or more diastereomers, a tautomer, a mixture of two or more tautomers, or an isotopic variant thereof.

In certain embodiments, R^{E} is a moiety having the structure of:

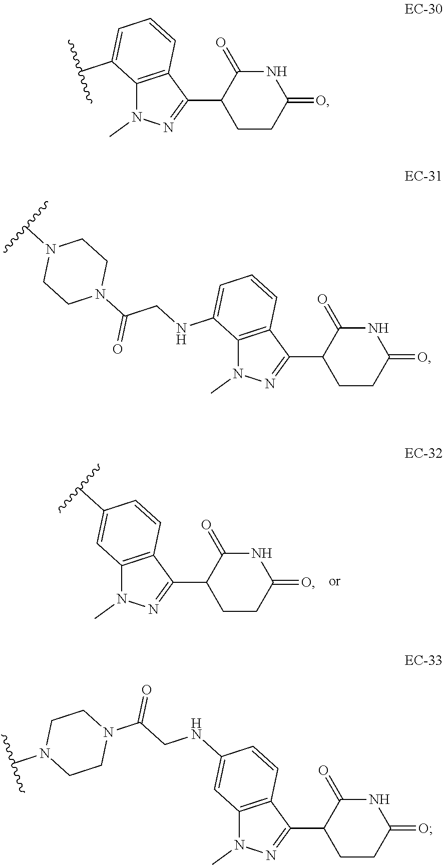 [SMILES: *c1cccc2c(C3CCC(=O)NC3=O)nn(C)c12 ; *N1CCN(C(=O)CNc2cccc3c(C4CCC(=O)NC4=O)nn(C)c23)CC1 ; *c1ccc2c(C3CCC(=O)NC3=O)nn(C)c2c1 ; "or" ; *N1CCN(C(=O)CNc2ccc3c(C4CCC(=O)NC4=O)nn(C)c3c2)CC1]

or an enantiomer, a mixture of enantiomers, a tautomer, a mixture of two or more tautomers, or an isotopic variant thereof.

In one embodiment, in a compound of any one of Formulae (I) to (XVI), R^{E} is a moiety having the structure of Formula EC-30, or an enantiomer, a mixture of enantiomers, a diastereomer, a mixture of two or more diastereomers, a tautomer, a mixture of two or more tautomers, or an isotopic variant thereof. In another embodiment, in a compound of any one of Formulae (I) to (XVI), R^{E} is a moiety having the structure of Formula EC-31, or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula EC-32, or an enantiomer, a mixture of enantiomers, a diastereomer, a mixture of two or more diastereomers, a tautomer, a mixture of two or more tautomers, or an isotopic variant thereof. In still another embodiment, in a compound of any one of Formulae (I) to (XVI), R^{E} is a moiety having the structure of Formula EC-33, or an enantiomer, a mixture of enantiomers, a diastereomer, a mixture of two or more diastereomers, a tautomer, a mixture of two or more tautomers, or an isotopic variant thereof.

In certain embodiments, R^{E} is a moiety having the structure of.

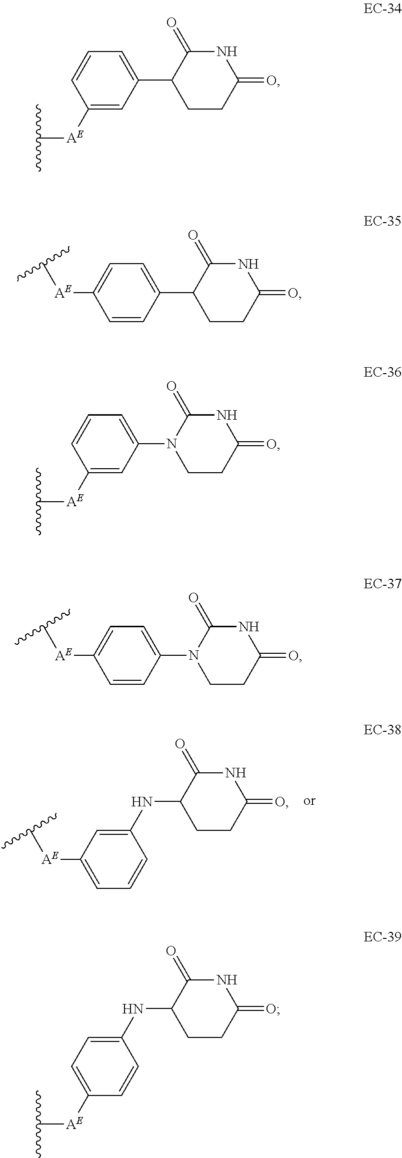

or an enantiomer, a mixture of enantiomers, a tautomer, a mixture of two or more tautomers, or an isotopic variant thereof; wherein A^{E} is as defined herein.

In one embodiment, in a compound of any one of Formulae (I) to (XVI), R^{E} is a moiety having the structure of Formula EC-34, or an enantiomer, a mixture of enantiomers, a diastereomer, a mixture of two or more diastereomers, a tautomer, a mixture of two or more tautomers, or an isotopic variant thereof. In another embodiment, in a compound of any one of Formulae (I) to (XVI), R^{E} is a moiety having the structure of Formula EC-35, or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula EC-36, or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula EC-37, or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula EC-38, or an enantiomer, a mixture of enantiomers, a diastereomer, a mixture of two or more diastereomers, a tautomer, a mixture of two or more tautomers, or an isotopic variant thereof. In still another embodiment, in a compound of any one of Formulae (I) to (XVI), R^{E} is a moiety having the structure of Formula EC-39, or an enantiomer, a mixture of enantiomers, a diastereomer, a mixture of two or more diastereomers, a tautomer, a mixture of two or more tautomers, or an isotopic variant thereof.

In certain embodiments, R^{E} is a moiety having the structure of.

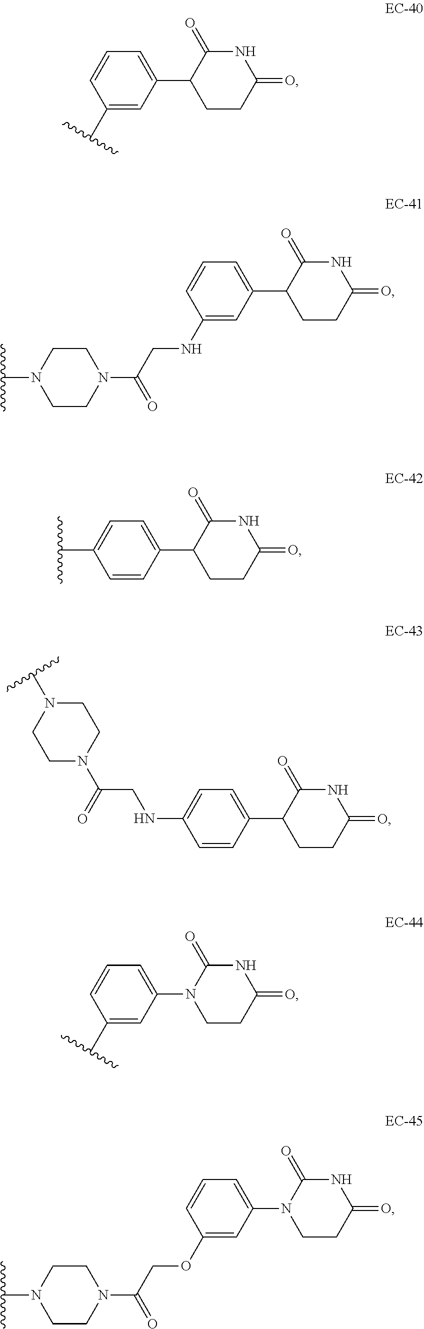 [SMILES: *c1cccc(C2CCC(=O)NC2=O)c1 ; *N1CCN(C(=O)CNc2cccc(C3CCC(=O)NC3=O)c2)CC1 ; *c1ccc(C2CCC(=O)NC2=O)cc1 ; *N1CCN(C(=O)CNc2ccc(C3CCC(=O)NC3=O)cc2)CC1 ; *c1cccc(N2CCC(=O)NC2=O)c1 ; *N1CCN(C(=O)COc2cccc(N3CCC(=O)NC3=O)c2)CC1]

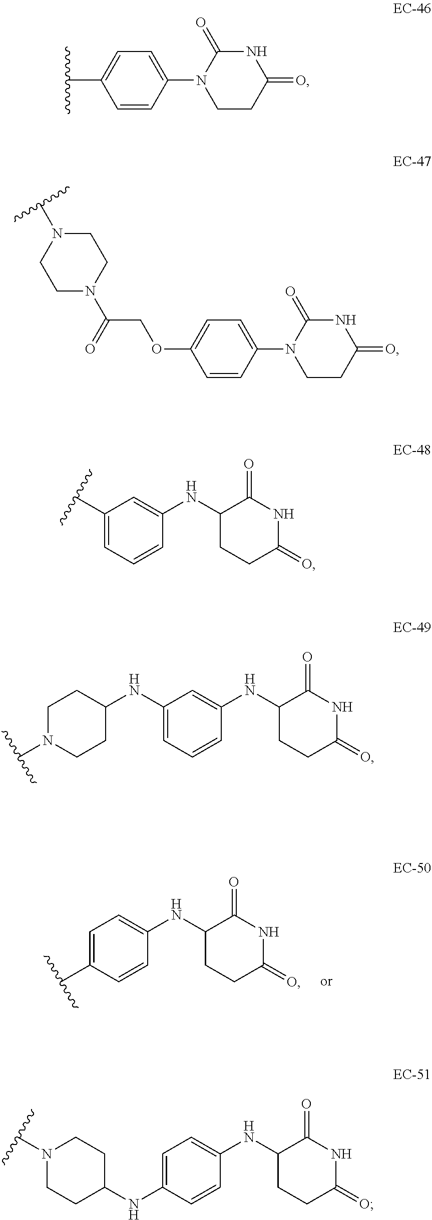

or an enantiomer, a mixture of enantiomers, a tautomer, a mixture of two or more tautomers, or an isotopic variant thereof.

In one embodiment, in a compound of any one of Formulae (I) to (XVI), R^{E} is a moiety having the structure of Formula EC-40, or an enantiomer, a mixture of enantiomers, a diastereomer, a mixture of two or more diastereomers, a tautomer, a mixture of two or more tautomers, or an isotopic variant thereof. In another embodiment, in a compound of any one of Formulae (I) to (XVI), R^{E} is a moiety having the structure of Formula EC-41, or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula EC-42, or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula EC-43, or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula EC-44, or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula EC-45, or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula EC-46, or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula EC-47, or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula EC-48, or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula EC-48, or an enantiomer, a mixture of enantiomers, a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula EC-50, or an enantiomer, a mixture of enantiomers, a diastereomer, a mixture of two or more diastereomers, a tautomer, a mixture of two or more tautomers, or an isotopic variant thereof. In still another embodiment, in a compound of any one of Formulae (I) to (XVI), R^{E} is a moiety having the structure of Formula EC-51, or an enantiomer, a mixture of enantiomers, a diastereomer, a mixture of two or more diastereomers, a tautomer, a mixture of two or more tautomers, or an isotopic variant thereof.

In certain embodiments, R^{E} is a moiety of an IAP E3 ligand.

In certain embodiments, R^{E} is a moiety having the structure of Formula (EI-I):

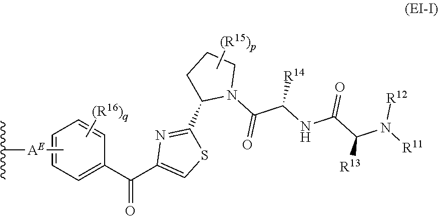

or a diastereomer, a mixture of two or more diastereomers, a tautomer, a mixture of two or more tautomers, or an isotopic variant thereof; wherein:
R^{I1} and R^{I2} are each independently hydrogen, C₁₋₆ alkyl, or C₃₋₁₀ cycloalkyl;
R^{I3} and R^{I4} are each independently C₁₋₆ alkyl, C₃₋₁₀ cycloalkyl, or heterocyclyl;
each R^{I5} and R^{I6} is independently (i) deuterium, cyano, halo, nitro, or oxo; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NRi R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
p is an integer of 0, 1, 2, 4, or 5;
q is an integer of 0, 1, 2, 3, or 4; and
each R^{1a}, R^{1b}, R^{1c}, R^{1d}, and A^{E} is as defined herein;
wherein each alkyl, heteroalkyl, alkenyl, alkynyl, cycloalkyl, aryl, aralkyl, heteroaryl, and heterocyclyl is optionally substituted with one or more substituents Q.

In certain embodiments, R^{E} is a moiety having the structure of Formula (EI-II):

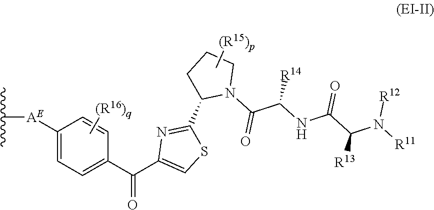

or a diastereomer, a mixture of two or more diastereomers, a tautomer, a mixture of two or more tautomers, or an isotopic variant thereof; wherein R^{I1}, R^{I2}, R^{I3}, R^{I4}, R^{I5}, R^{I6}, A^{E}, p, and q are each as defined herein.

In certain embodiments, R^{E} is a moiety having the structure of Formula (EI-III):

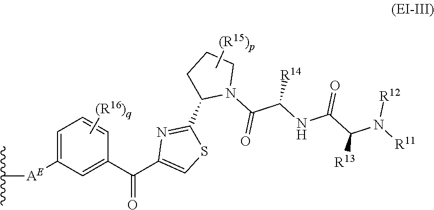

or a diastereomer, a mixture of two or more diastereomers, a tautomer, a mixture of two or more tautomers, or an isotopic variant thereof; wherein R^{I1}, R^{I2}, R^{I3}, R^{I4}, R^{I5}, R^{I6}, A^{E}, p, and q are each as defined herein.

In one embodiment, in Formula (EI-I), (EI-II), or (EI-III), R^{I1} is hydrogen; R^{I2} is C₁₋₆ alkyl; R^{I3} is C₁₋₆ alkyl; R^{I4} is C₁₋₆ alkyl or C₃₋₁₀ cycloalkyl; R^{I5} is —OR^{1a}; R^{I6} is halo; and p and q are each independently an integer of 0 or 1. In another embodiment, in Formula (EI-I), (EI-II), or (EI-III), R^{I1} is hydrogen; R^{I2} is C₁₋₆ alkyl; R^{I3} is C₁₋₆ alkyl; R^{I4} is C₃₋₁₀ cycloalkyl; R^{I5} is —OR^{1a}; R^{I6} is halo; and p and q are each independently an integer of 0 or 1. In yet another embodiment, in Formula (EI-I), (EI-II), or (EI-III), R^{I1} is hydrogen; R^{I2} and R^{I3} are each methyl; R^{I4} is cyclohexyl; R^{I5} is hydroxyl; R^{I6} is fluoro; and p and q are each independently an integer of 0 or 1.

In certain embodiments, R^{E} is a moiety having the structure of Formula (EI-IV).

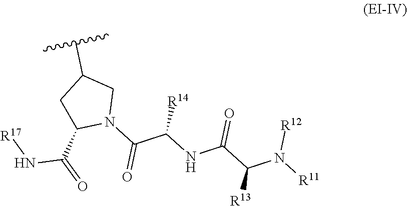

or a diastereomer, a mixture of two or more diastereomers, a tautomer, a mixture of two or more tautomers, or an isotopic variant thereof; wherein R¹⁷ is C₃₋₁₀ cycloalkyl, C₆₋₁₄ aryl, C₇₋₁₅ aralkyl, heteroaryl, or heterocyclyl, each optionally substituted with one or more substituents Q; and R^{I1}, R^{I2}, R^{I3}, and R^{I4} are each as defined herein.

In one embodiment, in Formula (EI-IV), R^{I1} is hydrogen; R^{I2} is C₁₋₆ alkyl; R^{I3} is C₁₋₆ alkyl; R^{I4} is C₁₋₆ alkyl or C₃₋₁₀ cycloalkyl; and R¹⁷ is C₃₋₁₀ cycloalkyl or C₆₋₁₄ aryl. In another embodiment, in Formula (EI-IV), R^{I1} is hydrogen; R^{I2} is C₁₋₆ alkyl; R^{I3} is C₁₋₆ alkyl; R^{I4} is C₃₋₁₀ cycloalkyl; and R^{I7} is C₃₋₁₀ cycloalkyl or C₆₋₁₄ aryl. In yet another embodiment, in Formula (EI-IV), R^{I1} is hydrogen; R^{I2} is methyl; R^{I3} is methyl; R^{I4} is cyclohexyl; and R^{I7} is 1,2,3,4-tetrahydronaphthalenyl. In still another embodiment, in Formula (EI-IV), R^{I1} is hydrogen; R^{I2} is methyl; R^{I3} is methyl; R^{I4} is cyclohexyl; and R^{I7} is 1,2,3,4-tetrahydronaphthalen-1-yl.

In certain embodiments, R^{E} is a moiety having the structure of Formula (EI-V):

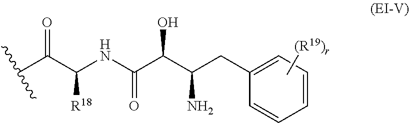

or a diastereomer, a mixture of two or more diastereomers, a tautomer, a mixture of two or more tautomers, or an isotopic variant thereof; wherein:
R^{I8} is C₁₋₆ alkyl, C₃₋₁₀ cycloalkyl, or heterocyclyl;
each R^{I9} is independently (i) deuterium, cyano, halo, nitro, or oxo; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
r is an integer of 0, 1, 2, 4, or 5; and
each R^{1a}, R^{1b}, R^{1c}, and R^{1d} is as defined herein;
wherein each alkyl, heteroalkyl, alkenyl, alkynyl, cycloalkyl, aryl, aralkyl, heteroaryl, and heterocyclyl is optionally substituted with one or more substituents Q.

In one embodiment, in Formula (EI-V), R^{I8} is C₁₋₆ alkyl or C₃₋₁₀ cycloalkyl; and r is an integer of 0 or 1. In another embodiment, in Formula (EI-V), R^{I8} is C₁₋₆ alkyl and r is an integer of 0. In yet another embodiment, in Formula (EI-V), R^{I8} is isobutyl and r is an integer of 0.

In one embodiment, in a compound of any one of Formulae (I) to (XVI), R^{E} is a moiety having the structure of Formula (EI-I), or a diastereomer, a mixture of two or more diastereomers, a tautomer, a mixture of two or more tautomers, or an isotopic variant thereof. In another embodiment, in a compound of any one of Formulae (I) to (XVI), R^{E} is a moiety having the structure of Formula (EI-II), or a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EI-III), or a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EI-IV), or a diastereomer, a mixture of two or more diastereomers, a tautomer, a mixture of two or more tautomers, or an isotopic variant thereof. In still another embodiment, in a compound of any one of Formulae (I) to (XVI), R^{E} is a moiety having the structure of Formula (EI-V), or a diastereomer, a mixture of two or more diastereomers, a tautomer, a mixture of two or more tautomers, or an isotopic variant thereof.

In certain embodiments, R^{E} is a moiety of an IAP E3 ligand having the structure of:

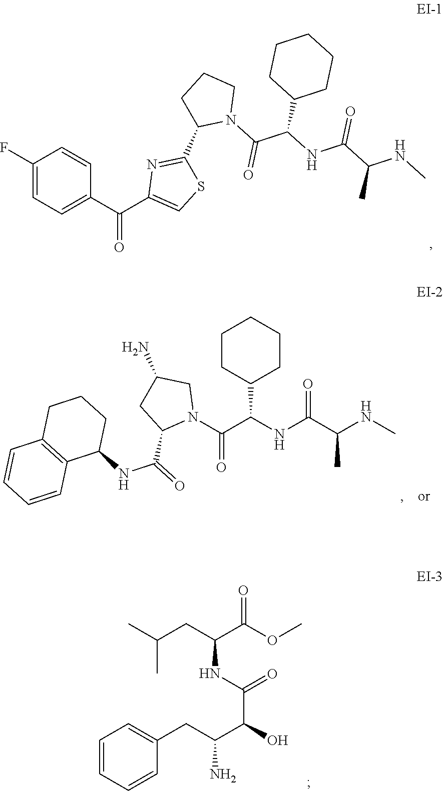 [SMILES: CN[C@@H](C)C(=O)N[C@H](C(=O)N1CCC[C@H]1c1nc(C(=O)c2ccc(F)cc2)cs1)C1CCCCC1 ; CN[C@@H](C)C(=O)N[C@H](C(=O)N1C[C@@H](N)C[C@H]1C(=O)N[C@@H]1CCCc2ccccc21)C1CCCCC1 ; COC(=O)[C@H](CC(C)C)NC(=O)[C@@H](O)[C@H](N)Cc1ccccc1]

or a diastereomer, a mixture of two or more diastereomers, a tautomer, a mixture of two or more tautomers, or an isotopic variant thereof.

In certain embodiments, R^{E} is a moiety having the structure of:

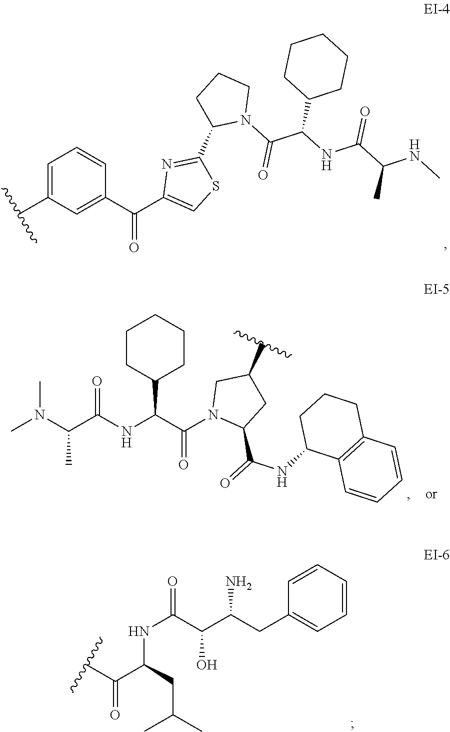

or a diastereomer, a mixture of two or more diastereomers, a tautomer, a mixture of two or more tautomers, or an isotopic variant thereof.

In one embodiment, in a compound of any one of Formulae (I) to (XVI), R^{E} is a moiety of compound EI-1, or a diastereomer, a mixture of two or more diastereomers, a tautomer, a mixture of two or more tautomers, or an isotopic variant thereof. In another embodiment, in a compound of any one of Formulae (I) to (XVI), R^{E} is a moiety of compound EI-2, or a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula EI-3, or a diastereomer, a mixture of two or more diastereomers, a tautomer, a mixture of two or more tautomers, or an isotopic variant thereof.

In one embodiment, in a compound of any one of Formulae (I) to (XVI), R^{E} is a moiety having the structure of Formula EI-4, or a diastereomer, a mixture of two or more diastereomers, a tautomer, a mixture of two or more tautomers, or an isotopic variant thereof. In another embodiment, in a compound of any one of Formulae (I) to (XVI), R^{E} is a moiety having the structure of Formula EI-5, or a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula EI-6, or a diastereomer, a mixture of two or more diastereomers, a tautomer, a mixture of two or more tautomers, or an isotopic variant thereof.

In certain embodiments, R^{E} is a moiety of an MDM2 E3 ligand.

In certain embodiments, R^{E} is a moiety having the structure of Formula (EM-I):

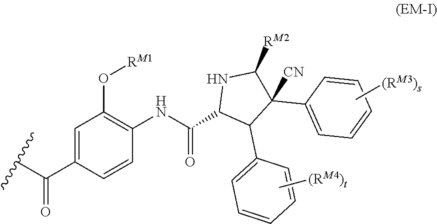

or a diastereomer, a mixture of two or more diastereomers, a tautomer, a mixture of two or more tautomers, or an isotopic variant thereof; wherein:
R^{M1} and R^{M2} are each independently C₁₋₆ alkyl or C₃₋₁₀ cycloalkyl;
each R^{M3} and R^{M4} is independently (i) deuterium, cyano, halo, nitro, or oxo; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
s and t are each independently an integer of 0, 1, 2, 3, 4, or 5; and
each R^{1a}, R^{1b}, R^{1c}, and R^{1d} is as defined herein;
wherein each alkyl, heteroalkyl, alkenyl, alkynyl, cycloalkyl, aryl, aralkyl, heteroaryl, and heterocyclyl is optionally substituted with one or more substituents Q.

In one embodiment, in Formula (EM-I), R^{M1} and R^{M2} are each independently C₁₋₆ alkyl; each R^{M3} and R^{M4} is independently deuterium or halo; and s and t are each independently an integer of 0, 1, or 2. In another embodiment, in Formula (EM-I), R^{M1} and R^{M2} are each independently methyl or 2,2-dimethylpropyl; each R^{M3} and R^{M4} is independently fluoro or chloro; and s and t are each independently an integer of 2.

In certain embodiments, R^{E} is a moiety having the structure of Formula (EM-II):

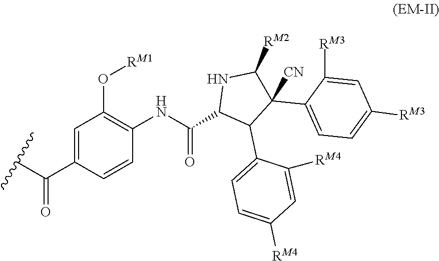

or a diastereomer, a mixture of two or more diastereomers, a tautomer, a mixture of two or more tautomers, or an isotopic variant thereof; wherein R^{M1}, R^{M2}, R^{M3}, and R^{M4} are each as defined herein.

In one embodiment, in Formula (EM-II), R^{M1} and R^{M2} are each independently C₁₋₆ alkyl; and each R^{M3} and R^{M4} is independently deuterium or halo. In another embodiment, in Formula (EM-II), R^{M1} and R^{M2} are each independently methyl or 2,2-dimethylpropyl; and each R^{M3} and R^{M4} is independently fluoro or chloro.

In certain embodiments, R^{E} is a moiety having the structure of Formula (EM-III):

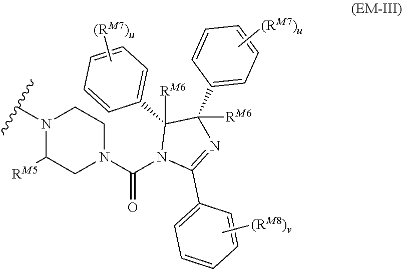

or a diastereomer, a mixture of two or more diastereomers, a tautomer, a mixture of two or more tautomers, or an isotopic variant thereof; wherein:
R^{M5} is hydrogen or oxo;
each R^{M6} is independently hydrogen, deuterium, or C₁₋₆ alkyl; and
each R^{M7} and R^{M8} is independently (i) deuterium, cyano, halo, nitro, or oxo; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each u and v is independently an integer of 0, 1, 2, 3, 4, or 5; and
each R^{1a}, R^{1b}, R^{1c}, and R^{1d} is as defined herein;
wherein each alkyl, heteroalkyl, alkenyl, alkynyl, cycloalkyl, aryl, aralkyl, heteroaryl, and heterocyclyl is optionally substituted with one or more substituents Q.

In one embodiment, in Formula (EM-III), R^{M5} is hydrogen or oxo; each R^{M6} is independently hydrogen, deuterium, or C₁₋₆ alkyl; each R^{M7} is independently halo; each R^{M8} is independently C₁₋₆ alkyl or —OC₁₋₆ alkyl; and each u and v is independently an integer of 0, 1, or 2. In another embodiment, in Formula (EM-III), R^{M5} is hydrogen or oxo; each R^{M6} is independently hydrogen or methyl; each R^{M7} is chloro; each R^{M8} is independently tert-butyl, methoxy, ethoxy, or isopropoxy; u is an integer of 0 or 1; and v is an integer of 1 or 2. In yet another embodiment, in Formula (EM-III), R^{M5} is hydrogen or oxo; each R^{M6} is independently hydrogen or methyl; each R^{M7} is chloro; each R^{M8} is independently tert-butyl, methoxy, ethoxy, or isopropoxy; u is an integer of 1; and v is an integer of 2.

In certain embodiments, R^{E} is a moiety having the structure of Formula (EM-IV):

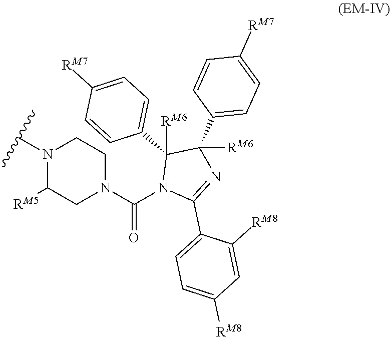

or a diastereomer, a mixture of two or more diastereomers, a tautomer, a mixture of two or more tautomers, or an isotopic variant thereof; wherein each R^{M5}, R^{M6}, R^{M7}, and R^{M8} is as defined herein.

In one embodiment, in Formula (EM-IV), R^{M5} is hydrogen or oxo; each R^{M6} is independently hydrogen, deuterium, or C₁₋₆ alkyl; each R^{M7} is independently halo; and each R^{M8} is independently C₁₋₆ alkyl or —OC₁₋₆ alkyl. In another embodiment, in Formula (EM-IV), R^{M5} is hydrogen or oxo; each R^{M6} is independently hydrogen or methyl; each R^{M7} is chloro; and each R^{M8} is independently tert-butyl, methoxy, ethoxy, or isopropoxy.

In one embodiment, in a compound of any one of Formulae (I) to (XVI), R^{E} is a moiety having the structure of Formula (EM-I), or a diastereomer, a mixture of two or more diastereomers, a tautomer, a mixture of two or more tautomers, or an isotopic variant thereof. In another embodiment, in a compound of any one of Formulae (I) to (XVI), R^{E} is a moiety having the structure of Formula (EM-II), or a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EM-III), or a diastereomer, a mixture of two or more diastereomers, a tautomer, a mixture of two or more tautomers, or an isotopic variant thereof. In still another embodiment, in a compound of any one of Formulae (I) to (XVI), R^{E} is a moiety having the structure of Formula (EM-IV), or a diastereomer, a mixture of two or more diastereomers, a tautomer, a mixture of two or more tautomers, or an isotopic variant thereof.

In certain embodiments, R^{E} is a moiety of an MDM2 E3 ligand having the structure of:

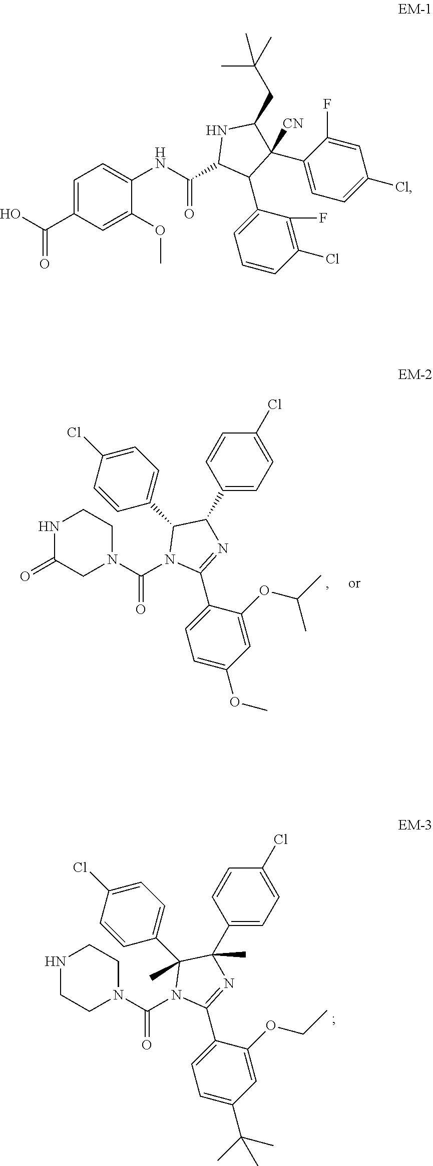

or a diastereomer, a mixture of two or more diastereomers, a tautomer, a mixture of two or more tautomers, or an isotopic variant thereof.

In one embodiment, in a compound of any one of Formulae (I) to (XVI), R^{E} is a moiety of compound EM-1, or a diastereomer, a mixture of two or more diastereomers, a tautomer, a mixture of two or more tautomers, or an isotopic variant thereof. In another embodiment, in a compound of any one of Formulae (I) to (XVI), R^{E} is a moiety of compound EM-2, or a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety of compound EM-3, or a diastereomer, a mixture of two or more diastereomers, a tautomer, a mixture of two or more tautomers, or an isotopic variant thereof.

In certain embodiments, R^{E} is a moiety having the structure of:

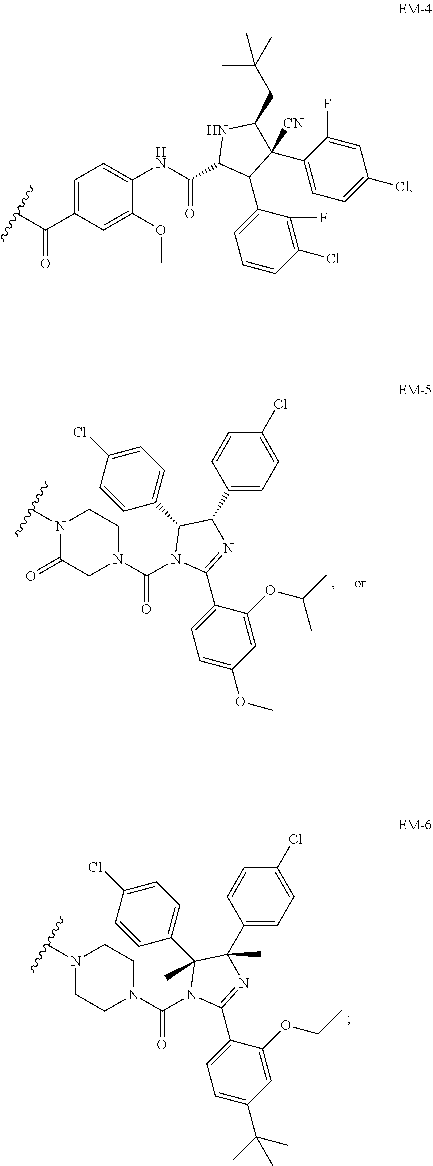

or a diastereomer, a mixture of two or more diastereomers, a tautomer, a mixture of two or more tautomers, or an isotopic variant thereof.

In one embodiment, in a compound of any one of Formulae (I) to (XVI), R^{E} is a moiety having the structure of Formula EM-4, or a diastereomer, a mixture of two or more diastereomers, a tautomer, a mixture of two or more tautomers, or an isotopic variant thereof. In another embodiment, in a compound of any one of Formulae (I) to (XVI), R^{E} is a moiety having the structure of Formula EM-5, or a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula EM-6, or a diastereomer, a mixture of two or more diastereomers, a tautomer, a mixture of two or more tautomers, or an isotopic variant thereof.

In certain embodiments, R^{E} is a moiety of a VHL E3 ligand.

In certain embodiments, R^{E} has the structure of Formula (EV-I):

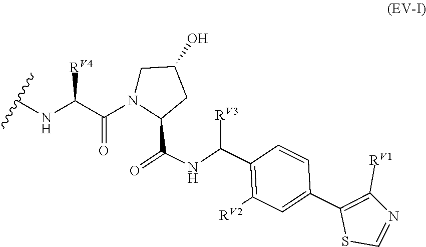

or a diastereomer, a mixture of two or more diastereomers, a tautomer, a mixture of two or more tautomers, or an isotopic variant thereof; wherein:
R^{V1}, R^{V3}, and R^{V4} are each independently hydrogen, deuterium, C₁₋₆ alkyl, or C₃₋₁₀ cycloalkyl; and
R^{V2} is hydrogen, deuterium, halo, hydroxyl, —OC₁₋₆ alkyl, or —OC₃₋₁₀ cycloalkyl;
wherein each alkyl and cycloalkyl is optionally substituted with one or more, in one embodiment, one, two, three, or four, substituents Q.

In certain embodiments, R^{E} has the structure of Formula (EV-II):

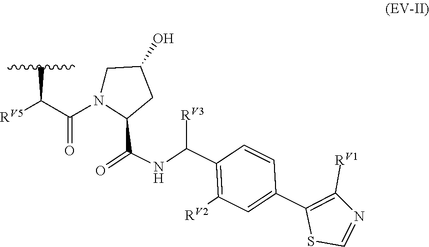

or a diastereomer, a mixture of two or more diastereomers, a tautomer, a mixture of two or more tautomers, or an isotopic variant thereof; wherein:
R^{V5} is —NHC(O)C₁₋₆ alkyl, —NHC(O)C₃₋₁₀ cycloalkyl, or heterocyclyl, wherein the alkyl, cycloalkyl, and heterocyclyl are each optionally substituted with one or more, in one embodiment, one, two, three, or four, substituents Q; and
R^{V1}, R^{V2}, and R^{V3} are each as defined herein.

In certain embodiments, R^{E} has the structure of Formula (EV-III):

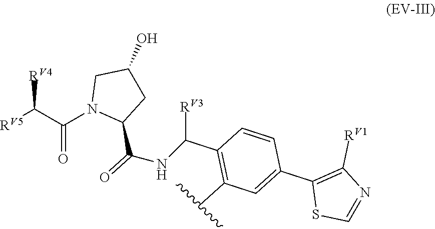

or a diastereomer, a mixture of two or more diastereomers, a tautomer, a mixture of two or more tautomers, or an isotopic variant thereof; wherein R^{V1}, R^{V3}, R^{V4}, and R^{V5} are each as defined herein.

In certain embodiments, in any one of Formulae (EV-I) to (EV-III),
R^{V1} is methyl;
R^{V2}, if present, is hydrogen;
R^{V3} is hydrogen;
R^{V4}, if present, is propyl, butyl, or cyclopropyl; and
R^{V5}, if present, is acetamido, cyclopropamido, or isoindolinyl;
wherein the propyl, butyl, cyclopropyl, acetamido, cyclopropamido, and isoindolinyl are each optionally substituted with cyano, fluoro, or trifluoromethyl.

In certain embodiments, in any one of Formulae (EV-I) to (EV-III),
R^{V1} is methyl;
R^{V2}, if present, is hydrogen;
R^{V3} is hydrogen;
R^{V4}, if present, is isopropyl, tert-butyl, cyclopropyl, 1-fluorocyclopropyl, or 1-trifuloromethylcyclopropyl; and
R^{V5}, if present, is acetamido, cyclopropamido, 1-cyanocyclopropamido, 1-fluorocyclopropamido, or 1-oxoisoindolin-2-yl.

In one embodiment, in a compound of any one of Formulae (I) to (XVI), R^{E} is a moiety having the structure of Formula (EV-I), or a diastereomer, a mixture of two or more diastereomers, a tautomer, a mixture of two or more tautomers, or an isotopic variant thereof. In another embodiment, in a compound of any one of Formulae (I) to (XVI), R^{E} is a moiety having the structure of Formula (EV-II), or a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula (EV-III), or a diastereomer, a mixture of two or more diastereomers, a tautomer, a mixture of two or more tautomers, or an isotopic variant thereof.

In certain embodiments, R^{E} is a moiety of a VHL E3 ligand having the structure of:

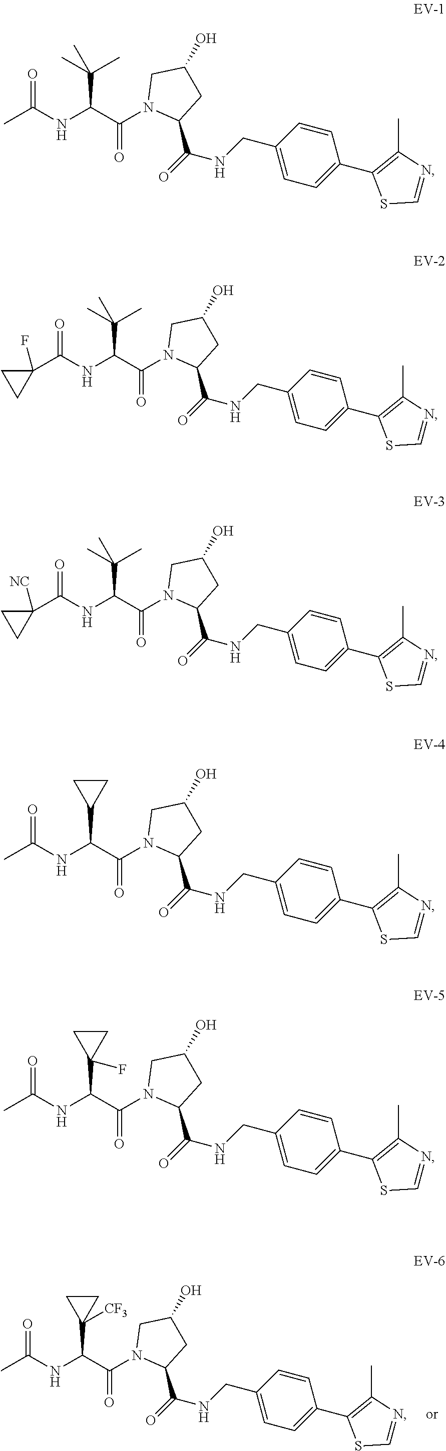

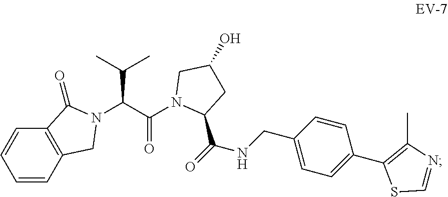 [SMILES: Cc1ncsc1-c1ccc(CNC(=O)[C@@H]2C[C@@H](O)CN2C(=O)[C@H](C(C)C)N2Cc3ccccc3C2=O)cc1]

or a diastereomer, a mixture of two or more diastereomers, a tautomer, a mixture of two or more tautomers, or an isotopic variant thereof.

In one embodiment, in a compound of any one of Formulae (I) to (XVI), R^{E} is a moiety of compound EV-1, or a diastereomer, a mixture of two or more diastereomers, a tautomer, a mixture of two or more tautomers, or an isotopic variant thereof. In another embodiment, in a compound of any one of Formulae (I) to (XVI), R^{E} is a moiety of compound EV-2, or a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety of compound EV-3, or a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety of compound EV-4, or a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety of compound EV-5, or a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety of compound EV-6, or a diastereomer, a mixture of two or more diastereomers, a tautomer, a mixture of two or more tautomers, or an isotopic variant thereof. In still another embodiment, in a compound of any one of Formulae (I) to (XVI), R^{E} is a moiety of compound EV-7, or a diastereomer, a mixture of two or more diastereomers, a tautomer, a mixture of two or more tautomers, or an isotopic variant thereof.

In certain embodiments, R^{E} is a moiety having the structure of:

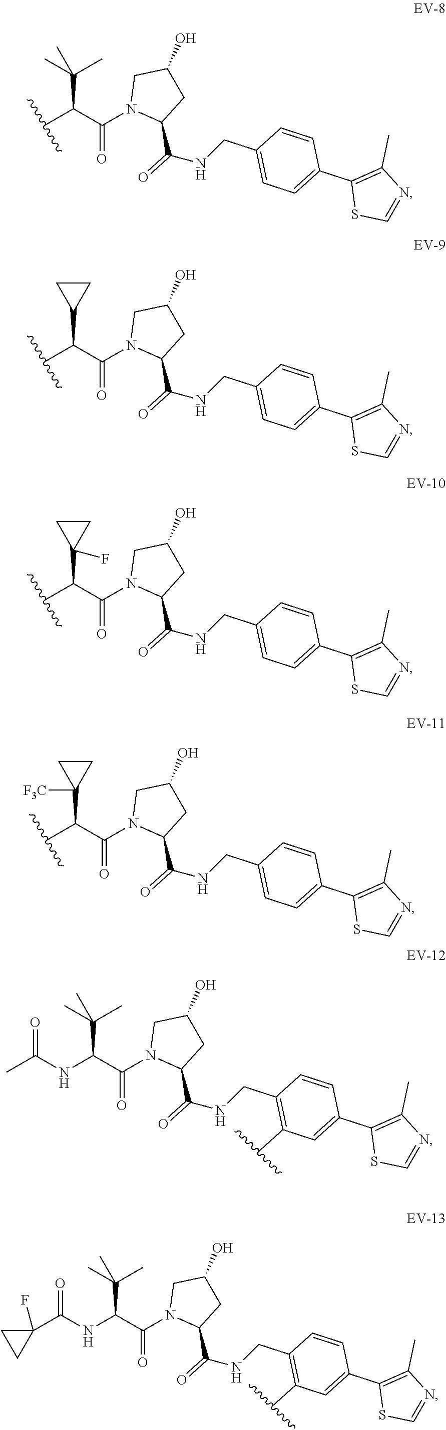 [SMILES: *[C@H](C(=O)N1C[C@H](O)C[C@H]1C(=O)NCc1ccc(-c2scnc2C)cc1)C(C)(C)C ; *[C@H](C(=O)N1C[C@H](O)C[C@H]1C(=O)NCc1ccc(-c2scnc2C)cc1)C1CC1 ; *[C@H](C(=O)N1C[C@H](O)C[C@H]1C(=O)NCc1ccc(-c2scnc2C)cc1)C1(F)CC1 ; *[C@H](C(=O)N1C[C@H](O)C[C@H]1C(=O)NCc1ccc(-c2scnc2C)cc1)C1(C(F)(F)F)CC1 ; *c1cc(-c2scnc2C)ccc1CNC(=O)[C@@H]1C[C@@H](O)CN1C(=O)[C@@H](NC(C)=O)C(C)(C)C ; *c1cc(-c2scnc2C)ccc1CNC(=O)[C@@H]1C[C@@H](O)CN1C(=O)[C@@H](NC(=O)C1(F)CC1)C(C)(C)C]

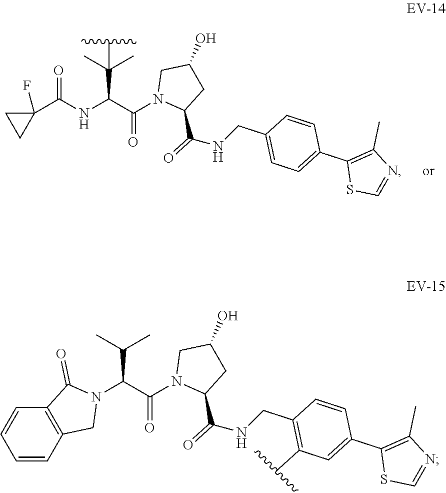 [SMILES: *C(C)(C)[C@H](NC(=O)C1(F)CC1)C(=O)N1C[C@H](O)C[C@H]1C(=O)NCc1ccc(-c2scnc2C)cc1 ; *c1cc(-c2scnc2C)ccc1CNC(=O)[C@@H]1C[C@@H](O)CN1C(=O)[C@H](C(C)C)N1Cc2ccccc2C1=O]

or a diastereomer, a mixture of two or more diastereomers, a tautomer, a mixture of two or more tautomers, or an isotopic variant thereof.

In certain embodiments, R^{E} is a moiety having the structure of:

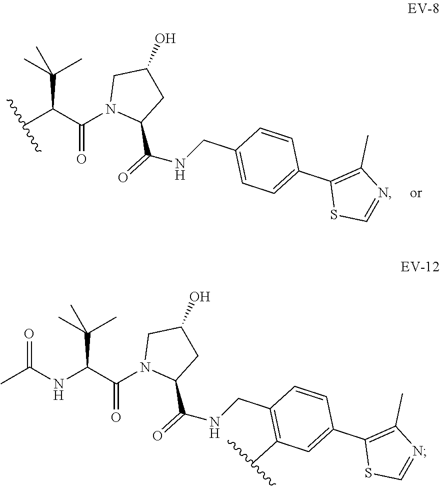 [SMILES: *[C@H](C(=O)N1C[C@H](O)C[C@H]1C(=O)NCc1ccc(-c2scnc2C)cc1)C(C)(C)C ; *c1cc(-c2scnc2C)ccc1CNC(=O)[C@@H]1C[C@@H](O)CN1C(=O)[C@@H](NC(C)=O)C(C)(C)C]

or a diastereomer, a mixture of two or more diastereomers, a tautomer, a mixture of two or more tautomers, or an isotopic variant thereof.

In one embodiment, in a compound of any one of Formulae (I) to (XVI), R^{E} is a moiety having the structure of Formula EV-8, or a diastereomer, a mixture of two or more diastereomers, a tautomer, a mixture of two or more tautomers, or an isotopic variant thereof. In another embodiment, in a compound of any one of Formulae (I) to (XVI), R^{E} is a moiety having the structure of Formula EV-9, or a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula EV-10, or a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula EV-11, or a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula EV-12, or a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula EV-13, or a diastereomer, a mixture of two or more diastereomers, a tautomer, a mixture of two or more tautomers, or an isotopic variant thereof. In yet another embodiment, in a compound of any one of Formulae (I) to (XVI), R^{E} is a moiety having the structure of Formula EV-14, or a diastereomer, a mixture of two or more diastereomers, a tautomer, a mixture of two or more tautomers, or an isotopic variant thereof. In still another embodiment, in a compound of any one of Formulae (I) to (XVI), R^{E} is a moiety having the structure of Formula EV-15, or a diastereomer, a mixture of two or more diastereomers, a tautomer, a mixture of two or more tautomers, or an isotopic variant thereof.

In one embodiment, provided herein is a compound of Formula (XVII):

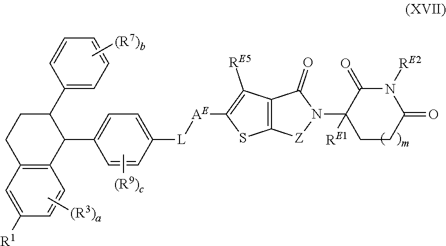

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R⁹, R^{E1}, R^{E2}, R^{E5}, A^{E}, L, Z, a, b, c, and m are each as defined herein.

In another embodiment, provided herein is a compound of Formula (XVIII):

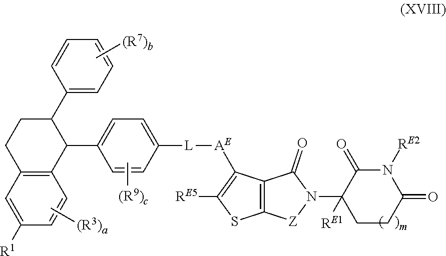

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R⁹, R^{E1}, R^{E2}, R^{E5}, A^{E}, L, Z, a, b, c, and m are each as defined herein.

In yet another embodiment, provided herein is a compound of Formula (XIX):

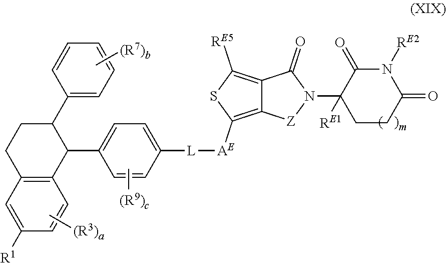

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R⁹, R^{E1}, R^{E2}, R^{E5}, A^{E}, L, Z, a, b, c, and m are each as defined herein.

In yet another embodiment, provided herein is a compound of Formula (XX):

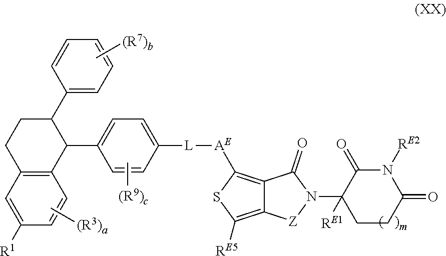

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R⁹, R^{E1}, R^{E2}, R^{E}5, A^{E}, L, Z, a, b, c, and m are each as defined herein.

In yet another embodiment, provided herein is a compound of Formula (XXI):

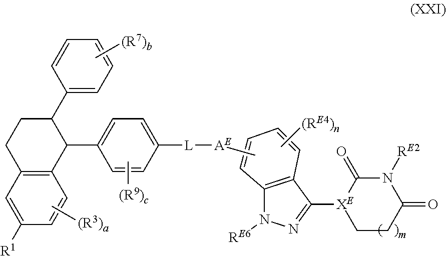

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R⁹, R^{E2}, R^{E4}, R^{E6}, A^{E}, L, X^{E}, a, b, c, m, and n are each as defined herein.

In yet another embodiment, provided herein is a compound of Formula (XXII):

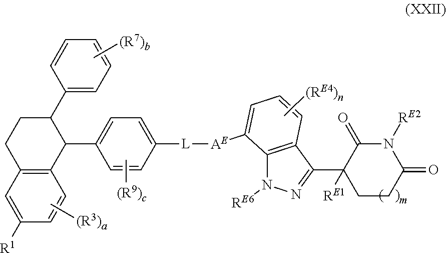

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R⁹, R^{E1}, R^{E2}, R^{E4}, R^{E6}, A^{E}, L, a, b, c, m, and n are each as defined herein.

In yet another embodiment, provided herein is a compound of Formula (XXIII):

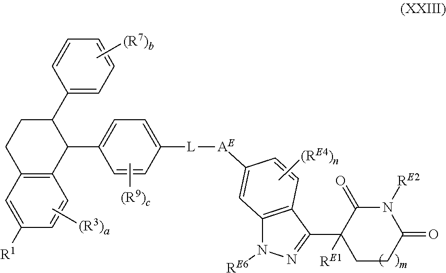

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R⁹, R^{E1}, R^{E2}, R^{E4}, R^{E6}, A^{E}, L, a, b, c, m, and n are each as defined herein.

In yet another embodiment, provided herein is a compound of Formula (XXIV):

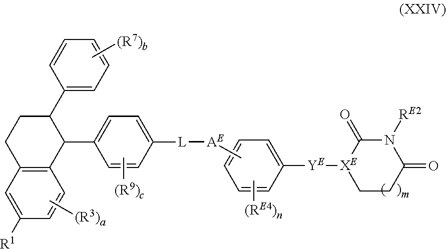

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R⁹, R^{E2}, R^{E4}, A^{E}, L, X^{E}, Y^{E}, a, b, c, m, and n are each as defined herein.

In yet another embodiment, provided herein is a compound of Formula (XXV):

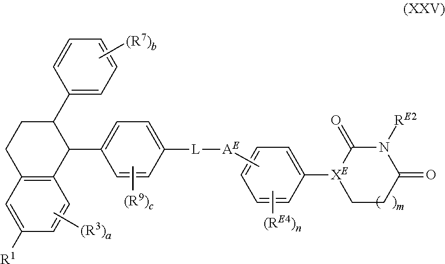

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R⁹, R^{E2}, R^{E4}, A^{E}, L, X^{E}, a, b, c, m, and n are each as defined herein.

In yet another embodiment, provided herein is a compound of Formula (XXVI):

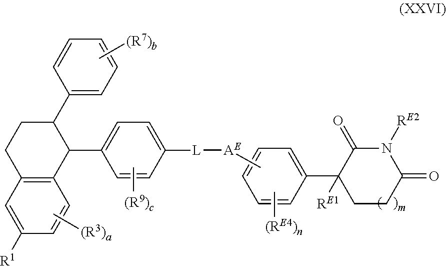

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R⁹, R^{E1}, R^{E2}, R^{E4}, A^{E}, L, a, b, c, m, and n are each as defined herein.

In yet another embodiment, provided herein is a compound of Formula (XXVII):

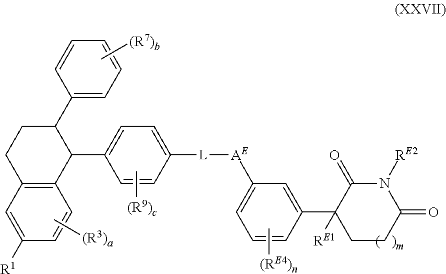

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R⁹, R^{E1}, R^{E2}, R^{E4}, A^{E}, L, a, b, c, m, and n are each as defined herein.

In yet another embodiment, provided herein is a compound of Formula (XXVIII):

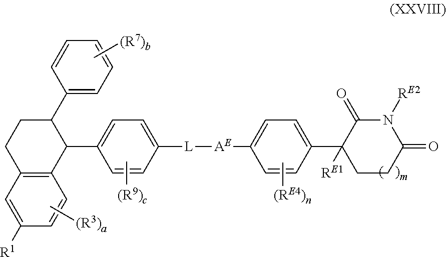

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R⁹, R^{E1}, R^{E2}, R^{E4}, A^{E}, L, a, b, c, m, and n are each as defined herein.

In yet another embodiment, provided herein is a compound of Formula (XXIX):

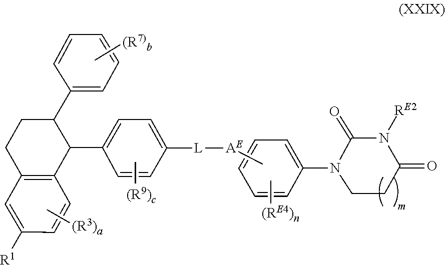

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R⁹, R^{E2}, R^{E4}, A^{E}, L, a, b, c, m, and n are each as defined herein.

In yet another embodiment, provided herein is a compound of Formula (XXX):

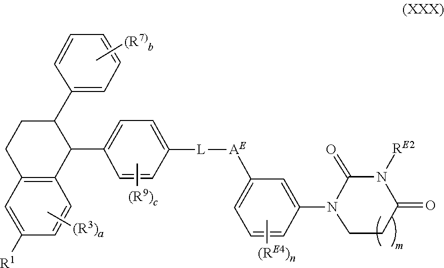

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R⁹, R^{E2}, R^{E4}, A^{E}, L, a, b, c, m, and n are each as defined herein.

In yet another embodiment, provided herein is a compound of Formula (XXXI):

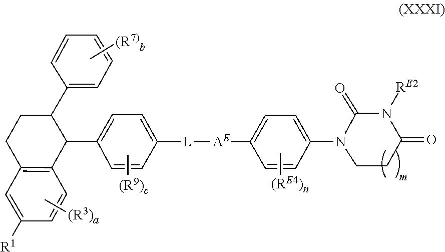

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R⁹, R^{E2}, R^{E4}, A^{E}, L, a, b, c, m, and n are each as defined herein.

In yet another embodiment, provided herein is a compound of Formula (XXXII):

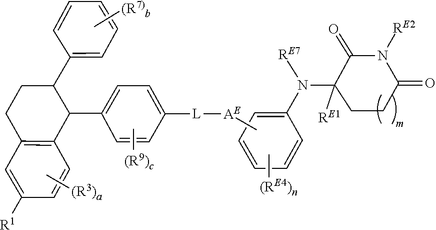

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R⁹, R^{E1}, R^{E2}, R^{E4}, R^{E7}, A^{E}, L, a, b, c, m, and n are each as defined herein.

In yet another embodiment, provided herein is a compound of Formula (XXXIII):

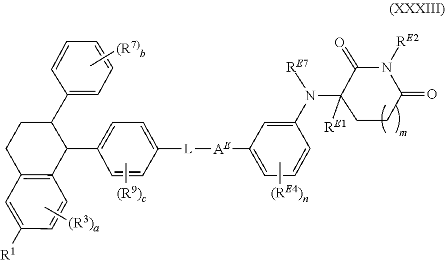

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R⁹, R^{E1}, R^{E2}, R^{E4}, R^{E7}, A^{E}, L, a, b, c, m, and n are each as defined herein.

In still another embodiment, provided herein is a compound of Formula (XXXIV):

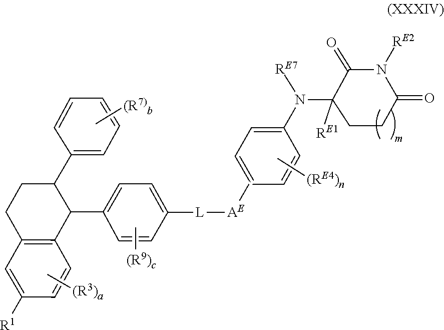

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R⁹, R^{E1}, R^{E2}, R^{E4}, R^{E7}, A^{E}, L, a, b, c, m, and n are each as defined herein.

In one embodiment, provided herein is a compound of Formula (XXXIX):

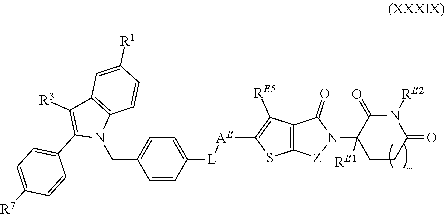

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R^{E1}, R^{E2}, R^{E5}, A^{E}, L, Z, and m are each as defined herein.

In another embodiment, provided herein is a compound of Formula (XL):

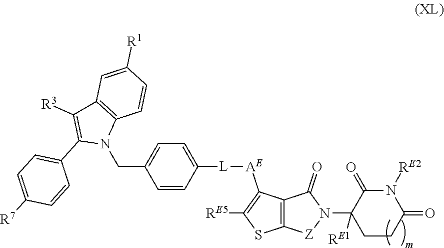

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R^{E1}, R^{E2}, R^{E5}, A^{E}, L, Z, and m are each as defined herein.

In yet another embodiment, provided herein is a compound of Formula (XLI):

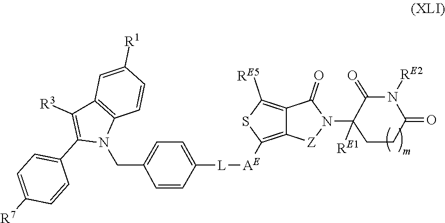

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R^{E1}, R^{E2}, R^{E5}, A^{E}, L, Z, and m are each as defined herein.

In yet another embodiment, provided herein is a compound of Formula (XLII):

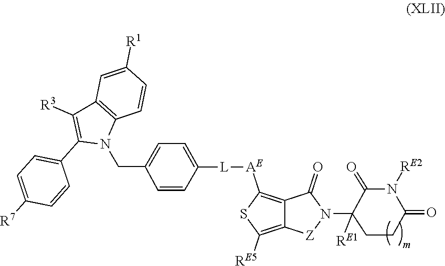

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R^{E1}, R^{E2}, R^{E}5, A^{E}, L, Z, and m are each as defined herein.

In yet another embodiment, provided herein is a compound of Formula (XLIII):

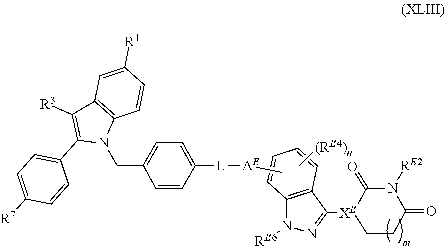

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R^{E2}, R^{E4}, R^{E6}, A^{E}, L, X^{E}, m, and n are each as defined herein.

In yet another embodiment, provided herein is a compound of Formula (XLIV):

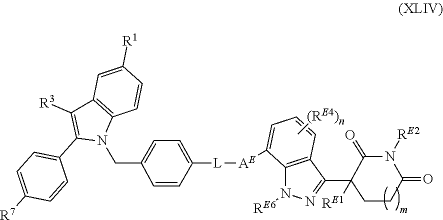

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R^{E1}, R^{E2}, R^{E4}, R^{E6}, A^{E}, L, m, and n are each as defined herein.

In yet another embodiment, provided herein is a compound of Formula (XLV):

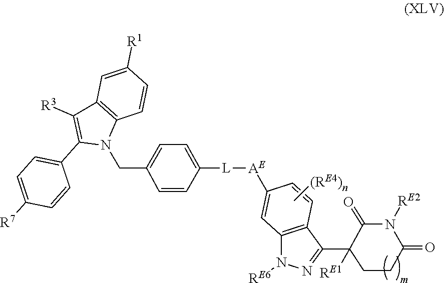

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R^{E1}, R^{E2}, R^{E4}, R^{E6}, A^{E}, L, m, and n are each as defined herein.

In yet another embodiment, provided herein is a compound of Formula (XLVI):

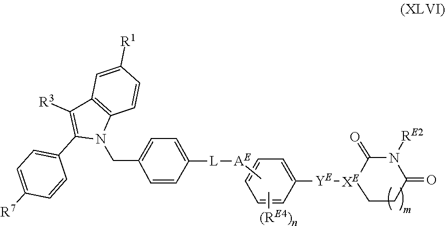

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R^{E2}, R^{E4}, A^{E}, L, X^{E}, Y^{E}, m, and n are each as defined herein.

In yet another embodiment, provided herein is a compound of Formula (XLVII):

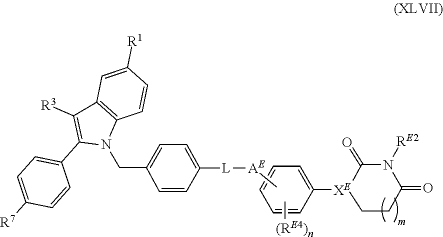

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R^{E2}, R^{E4}, A^{E}, L, X^{E}, m, and n are each as defined herein.

In yet another embodiment, provided herein is a compound of Formula (XLVIII):

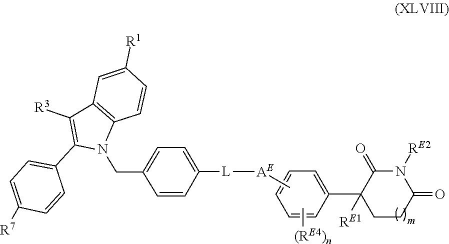

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R^{E1}, R^{E2}, R^{E4}, A^{E}, L, m, and n are each as defined herein.

In yet another embodiment, provided herein is a compound of Formula (XLIX):

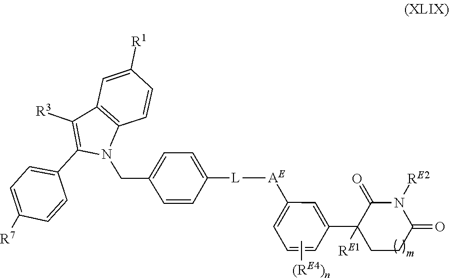

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R⁹, R^{E1}, R^{E2}, R^{E4}, A^{E}, L, a, b, c, m, and n are each as defined herein.

In yet another embodiment, provided herein is a compound of Formula (L):

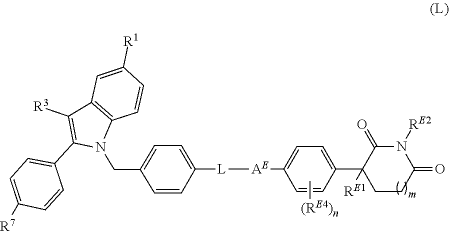

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R^{E1}, R^{E2}, R^{E4}, A^{E}, L, m, and n are each as defined herein.

In yet another embodiment, provided herein is a compound of Formula (LI):

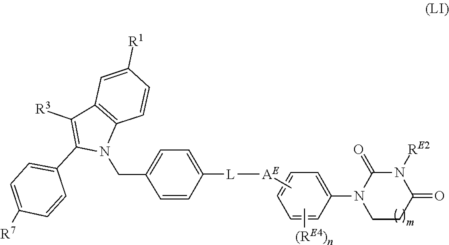

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R^{E2}, R^{E4}, A^{E}, L, m, and n are each as defined herein.

In yet another embodiment, provided herein is a compound of Formula (LII):

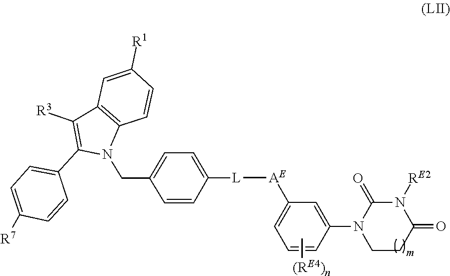

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R^{E2}, R^{E4}, A^{E}, L, m, and n are each as defined herein.

In yet another embodiment, provided herein is a compound of Formula (LIII):

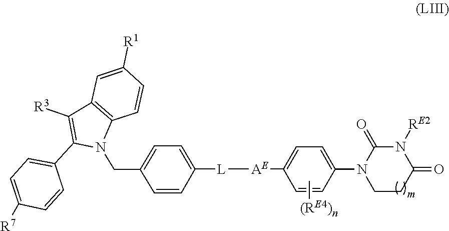

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R^{E2}, R^{E4}, A^{E}, L, m, and n are each as defined herein.

In yet another embodiment, provided herein is a compound of Formula (LIV):

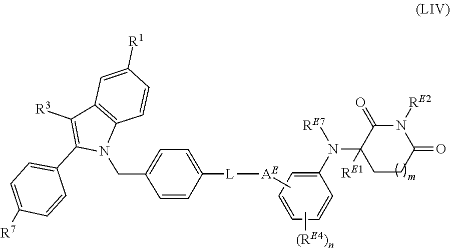

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R^{E1}, R^{E2}, R^{E4}, R^{E7}, A^{E}, L, m, and n are each as defined herein.

In yet another embodiment, provided herein is a compound of Formula (LV):

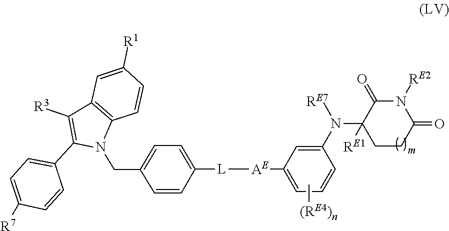

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R^{E1}, R^{E2}, R^{E4}, R^{E7}, A^{E}, L, m, and n are each as defined herein.

In still another embodiment, provided herein is a compound of Formula (LVI):

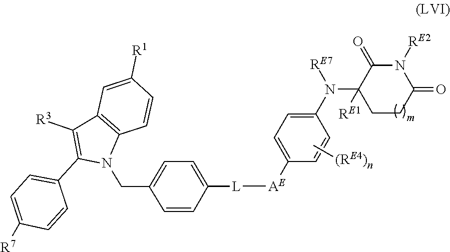

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁷, R^{E1}, R^{E2}, R^{E4}, R^{E7}, A^{E}, L, m, and n are each as defined herein.

In one embodiment, in any one of Formulae (XXXIX) to (LVI),
R¹ is hydrogen, deuterium, halo, —C(O)OR^{1a}, —OR^{1a}, or —NR^{1b}R^{1c};
R³ and R⁷ are each independently halo, C₁₋₆ alkyl, C₁₋₆ heteroalkyl, or —OR^{1a}; and
each R^{1a}, R^{1b}, and R^{1c} is as defined herein;
wherein each alkyl and heteroalkyl is optionally substituted with one or more substituents Q.

In another embodiment, in any one of Formulae (XXXIX) to (LVI),
R¹ is independently hydrogen, fluoro, iodo, —CO₂H, —OH, or —NH₂;
R³ is fluoro, chloro, hydroxyl, or methyl; and
R⁷ is fluoro, chloro, bromo, methyl, trifluoromethyl, ethyl, 2,2-filuoropropyl, isopropyl, isobutyl, t-butyl, cyclopropylmethyl, hydroxyl, methoxy, or trifluoromethoxy.

In yet another embodiment, in any one of Formulae (XXXIX) to (LVI),
R¹ is independently hydrogen, —CO₂H, or —OH;
R³ is fluoro, chloro, hydroxyl, or methyl; and
R⁷ is independently fluoro, chloro, bromo, methyl, trifluoromethyl, ethyl, 2,2-filuoropropyl, isopropyl, isobutyl, t-butyl, cyclopropylmethyl, hydroxyl, methoxy, or trifluoromethoxy.

In certain embodiments, L is a linker having the structure of —Z^{L}—(R^{L}—Z^{L})_{z}—, wherein:
each R^{L} is independently C₁₋₁₀ alkylene, C₂₋₁₀ alkenylene, C₂₋₁₀ alkynylene, C₃₋₁₀ cycloalkylene, C₆₋₁₄ arylene, heteroarylene, or heterocyclylene, each of which is optionally substituted with one or more substituents Q;
each Z^{L} is independently a bond, —C(O)—, —C(O)O—, —C(O)NR^{1b}—, —C(O)S—, —C(NR^{1a})NR^{1b}—, —C(S)—, —C(S)O—, —C(S)NR^{1b}—, —O—, —OC(O)O—, —OC(O)NR^{1b}—, —OC(O)S—, —OC(NR^{1a})NR^{1b}—, —OC(S)O—, —OC(S)NR^{1b}—, —OS(O)—, —OS(O)₂—, —OS(O)NR^{1b}—, —OS(O)₂NR^{1b}—, —NR^{1b}—, —NR^{1a}C(O)NR^{1b}—, —NR^{1a}C(O)S—, —NR^{1a}C(NR^{1d})NR^{1b}—, —NR^{1a}C(S)NR^{1b}—, —NR^{1a}S(O)NR^{1b}—, —NR^{1a}S(O)₂NR^{1b}—, —S—, —S(O)—, —S(O)₂—, —S(O)NR^{1b}—, or —S(O)₂NR^{1b}—; where each R^{1a}, R^{1b}, and R^{1d} is as defined herein; and
z is an integer of 1, 2, 3, 4, 5, 6, 7, 8, 9, or 10.

In certain embodiments, each R^{L} is independently C₁₋₁₀ alkylene, C₂₋₁₀ alkynylene, C₃₋₁₀ cycloalkylene, C₆₋₁₄ arylene, heteroarylene, or heterocyclylene, each optionally substituted with one or more substituents Q; each Z^{L} is independently a bond, —C(O)—, —C(O)NR^{1b}—, —C(NR^{1a})NR^{1b}—, —O—, —OC(O)NR^{1b}—, —NR^{1b}—, —NR^{1a}C(O)NR^{1b}—, —NR^{1a}C(NR^{1d})NR^{1b}, —NR^{1a}S(O)NR^{1b}—, —NR^{1a}S(O)₂NR^{1b}—, —S—, —S(O)—, —S(O)₂—, —S(O)NR^{1b}—, or —S(O)₂NR^{1b}—; and z is an integer of 1, 2, 3, 4, 5, 6, 7, 8, 9, or 10; where each R^{1a}, R^{1b}, and R^{1d} is as defined herein.

In certain embodiments, each R^{L} is independently C₁₋₁₀ alkylene, C₂₋₁₀ alkynylene, C₃₋₁₀ cycloalkylene, C₆₋₁₄ arylene, heteroarylene, or heterocyclylene, each optionally substituted with one or more substituents Q; each Z^{L} is independently a bond, —C(O)—, —C(O)NR^{1b}—, —O—, —OC(O)NR^{1b}—, —NR^{1b}—, —NR^{1a}C(O)NR^{1b}—, or —NR^{1a}C(NR^{1d})NR^{1b}—; and z is an integer of 1, 2, 3, 4, 5, 6, 7, or 8; where each R^{1a}, R^{1b}, and R^{1d} is as defined herein.

In certain embodiments, each R^{L} is independently methanediyl, ethanediyl, propanediyl, butanediyl, pentanediyl, hexanediyl, heptanediyl, octanediyl, nonanediyl, decanediyl, ethynediyl, cyclobutanediyl, cyclopentanediyl, cyclohexanediyl, cycloheptanediyl, bicyclo[2.2.2]octanediyl, phendiyl, pyrazoldiyl, imidazoldiyl, tetrazoldiyl, pyrimidindiyl, 5,6,7,8,9,10-hexahydrocycloocta[d]-pyridazindiyl, 1,3-dioxandiyl, piperazindiyl, piperidindiyl, or 3,9-diazaspiro[5.5]undecanediyl, each optionally substituted with one or more substituents Q; each Z^{L} is independently a bond, —C(O)—, —C(O)O—, —C(O)NH—, —OC(O)NH—, —O—, —NH—, —N(CH₃)—, or —NHC(O)NH—; and z is an integer of 1, 2, 3, 4, 5, 6, 7, or 8.

In certain embodiments, each R^{L} is independently methanediyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, ethyne-1,2-diyl, cyclobutane-1,3-diyl, cyclopentane-1,3-diyl, cyclohexane-1,3-diyl, cyclohexane-1,4-diyl, cycloheptane-1,3-diyl, cycloheptane-1,4-diyl, bicyclo[2.2.2]octane-1,4-diyl, phen-1,3-diyl, phen-1,4-diyl, pyrazol-1,3-diyl, pyrazol-1,4-diyl, imidazol-1,4-diyl, 1,2,3-triazol-1,4-diyl, pyrimidin-2,4-diyl, pyrimidin-2,5-diyl, 5,6,7,8,9,10-hexahydrocycloocta[d]-pyridazin-1,7-diyl, pyrazolidin-1,3-diyl, pyrazolidin-1,4-diyl, 1,3-dioxan-2,5-diyl, piperazin-1,4-diyl, piperidin-1,3-diyl, piperidin-1,4-diyl, or 3,9-diazaspiro[5.5]-undecane-3,9-diyl, each optionally substituted with one or more substituents Q; each Z^{L} is independently a bond, —C(O)—, —C(O)O—, —C(O)NH—, —OC(O)NH—, —O—, —NH—, —N(CH₃)—, or —NHC(O)NH—; and z is an integer of 1, 2, 3, 4, 5, 6, 7, or 8.

In certain embodiments, L is:

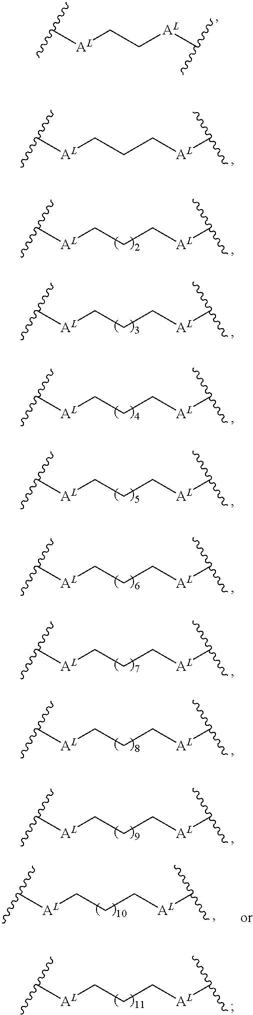

wherein each A^{L} is independently a bond, —O—, —NH—, or —N(CH₃)—.

In certain embodiments, L is:

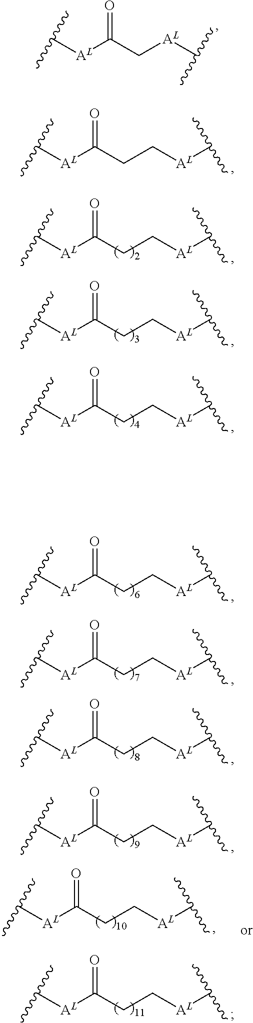

wherein each A^{L} is independently a bond, —O—, —NH—, or —N(CH₃)—.

In certain embodiments, L is:

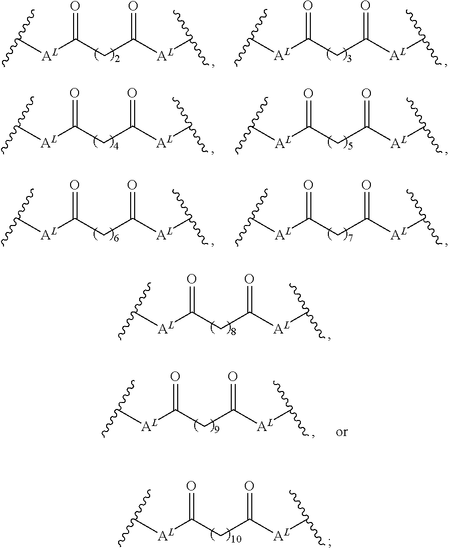

wherein each A^{L} is independently a bond, —O—, —NH—, or —N(CH₃)—.

In certain embodiments, L is:

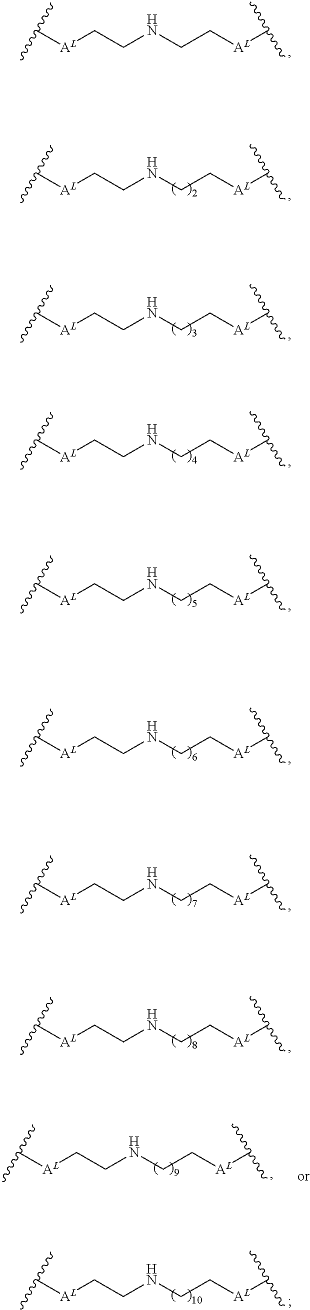

wherein each A^{L} is independently a bond, —O—, —NH—, or —N(CH₃)—; and wherein each amino (NH) group is optionally substituted with methyl.

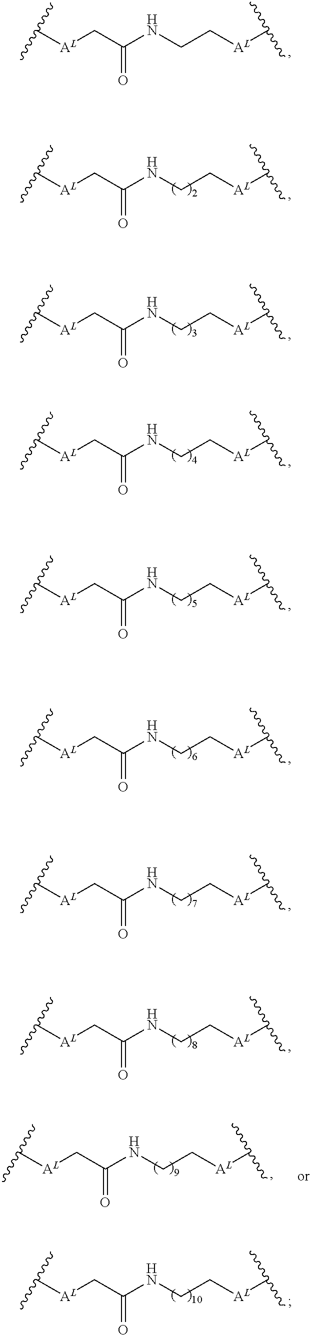

wherein each A^{L} is independently a bond, —O—, —NH—, or —N(CH₃)—; and wherein each amino (NH) group is optionally substituted with methyl.

In certain embodiments, L is:

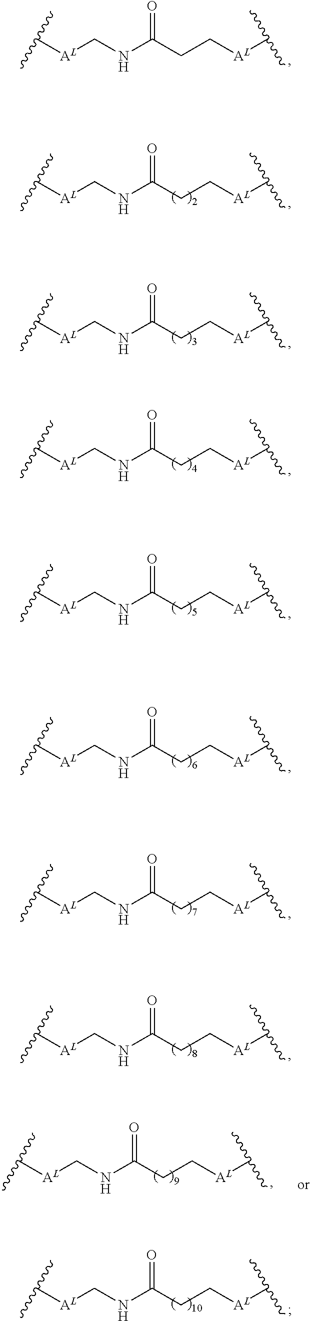

wherein each A^{L} is independently a bond, —O—, —NH—, or —N(CH₃)—; and wherein each amino group (NH) is optionally substituted with methyl.

In certain embodiments, L is:

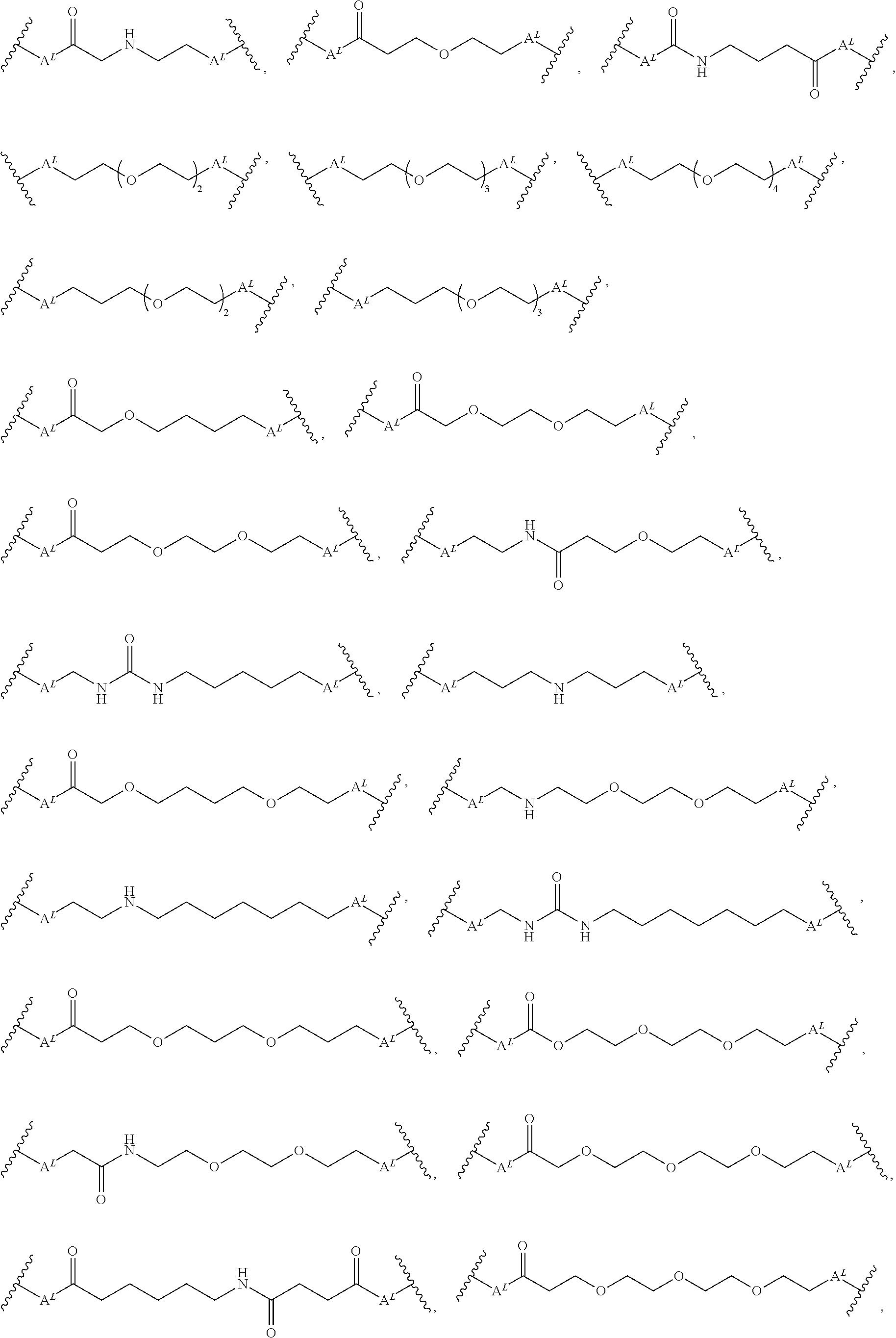

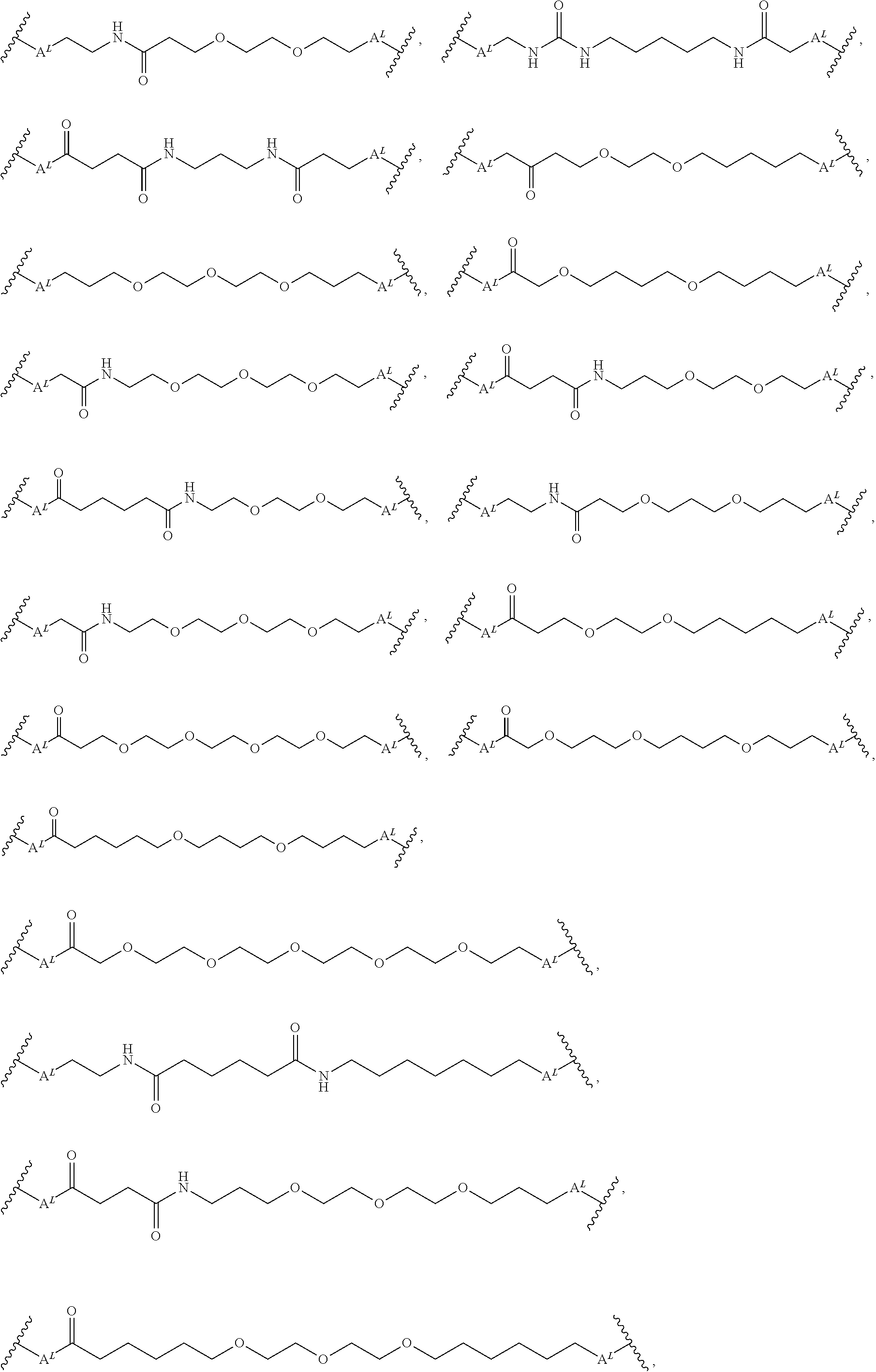

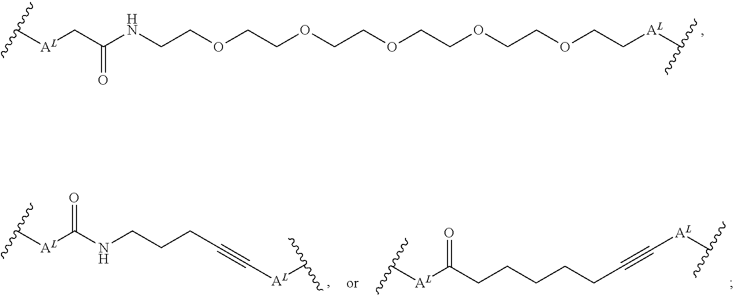

wherein each A^{L} is independently a bond, —O—, —NH—, or —N(CH₃)—; and wherein each amino (NH) group is optionally substituted with methyl.

In certain embodiments, L is:

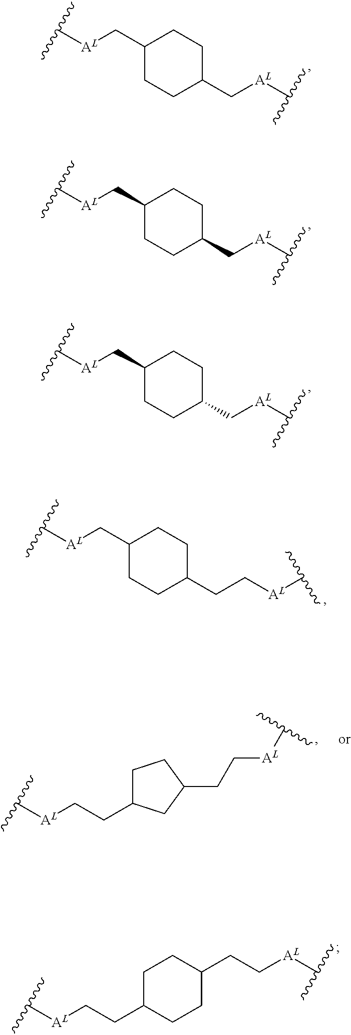

wherein each A^{L} is independently a bond, —O—, —NH—, or —N(CH₃)—.

In certain embodiments, L is:

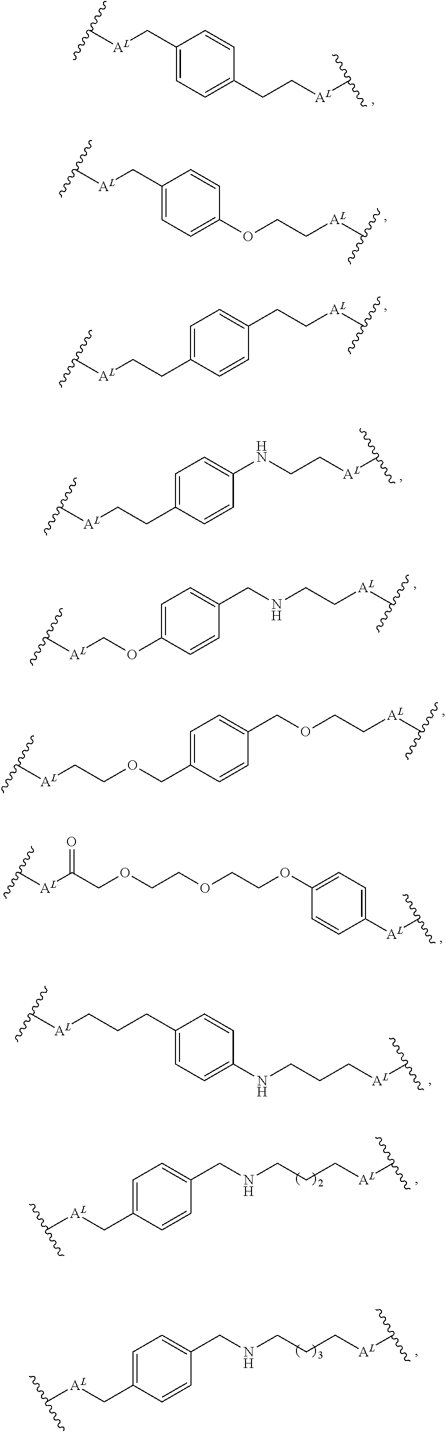

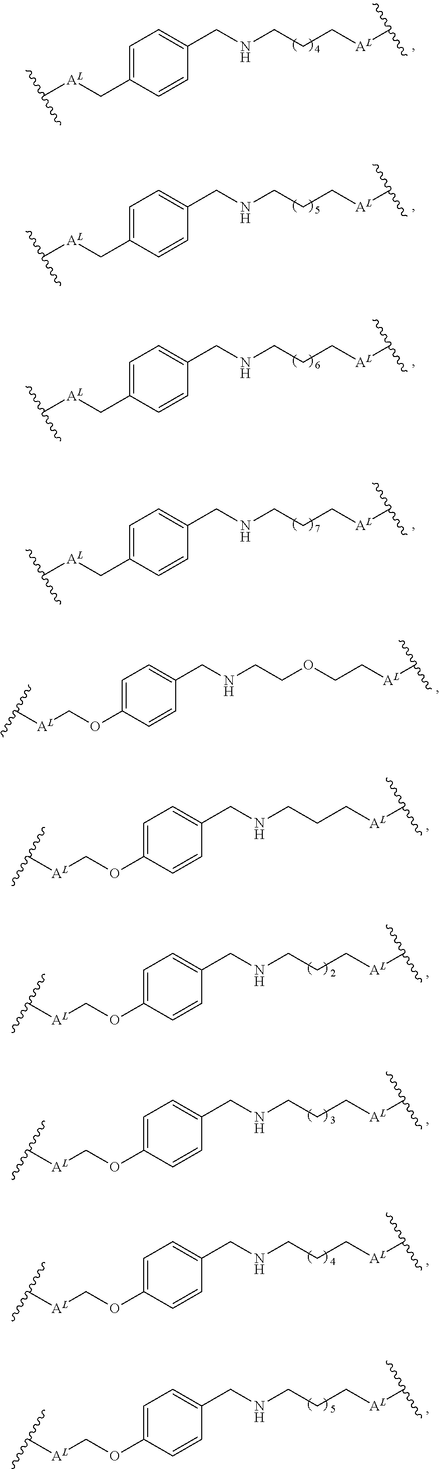

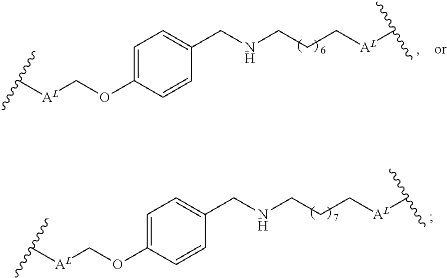

wherein each A^{L} is independently a bond, —O—, —NH—, or —N(CH₃)—; and wherein each amino group (NH) is optionally substituted with methyl.

In certain embodiments, L is:

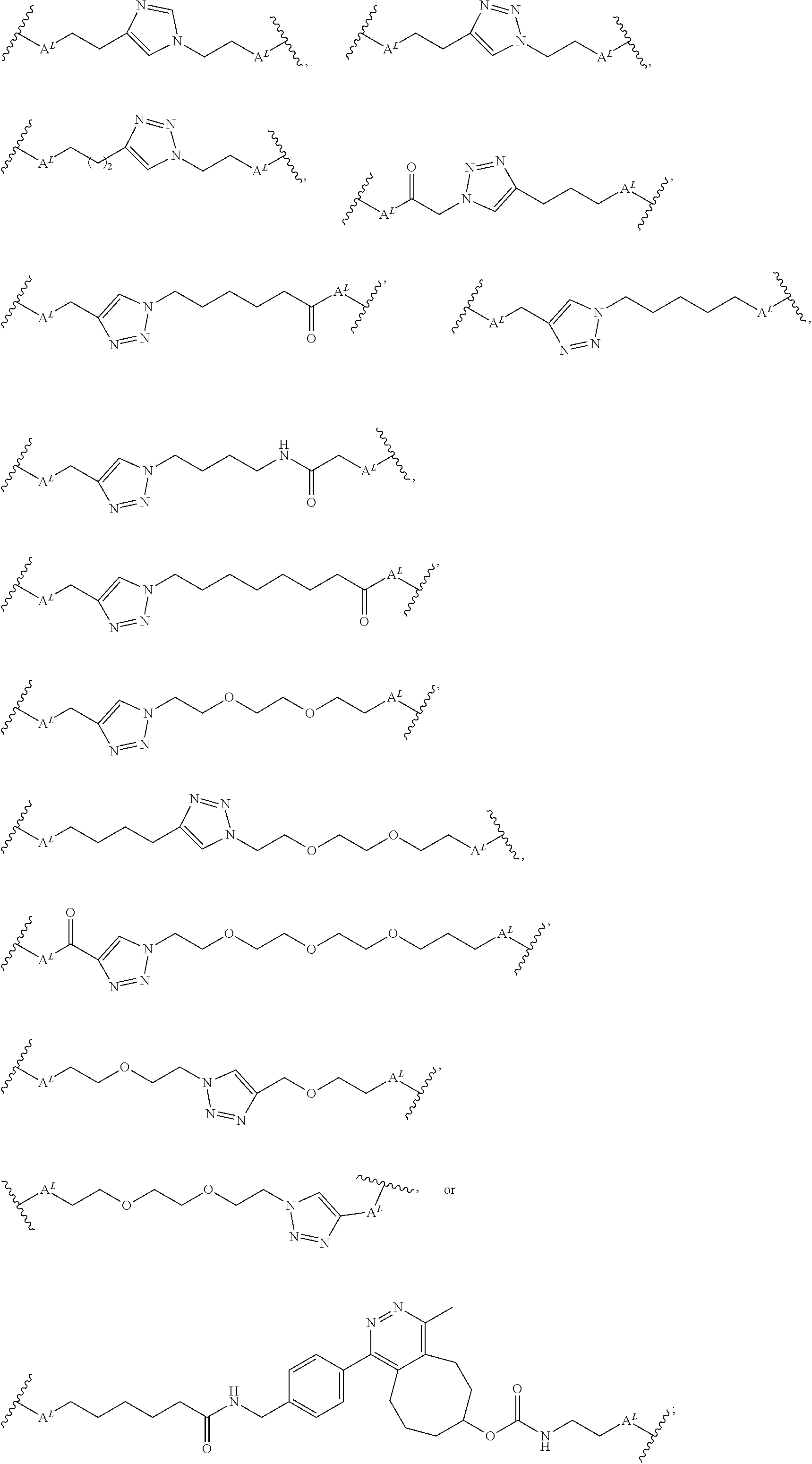

wherein each A^{L} is independently a bond, —O—, —NH—, or —N(CH₃)—; and wherein each amino group (NH) is optionally substituted with methyl.

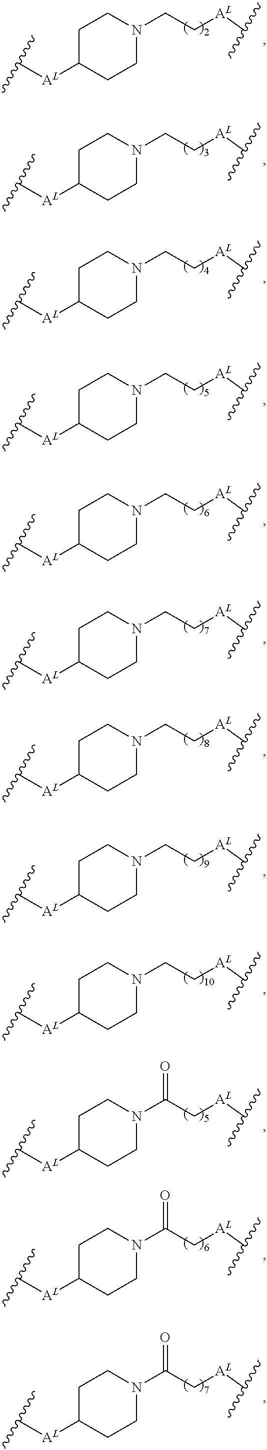

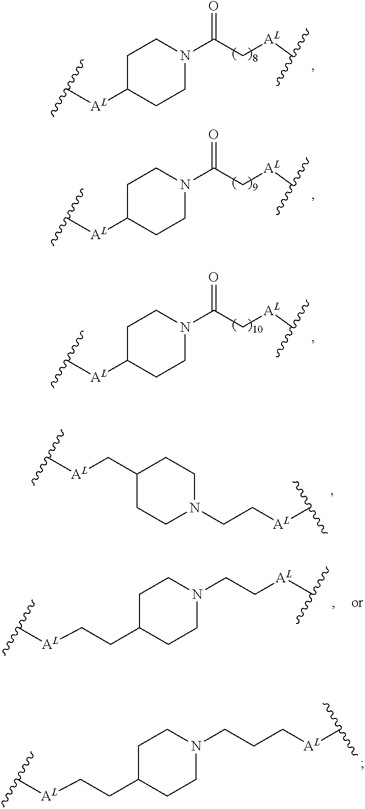

wherein each A^{L} is independently a bond, —O—, —NH—, or —N(CH₃)—.

In certain embodiments, L is:

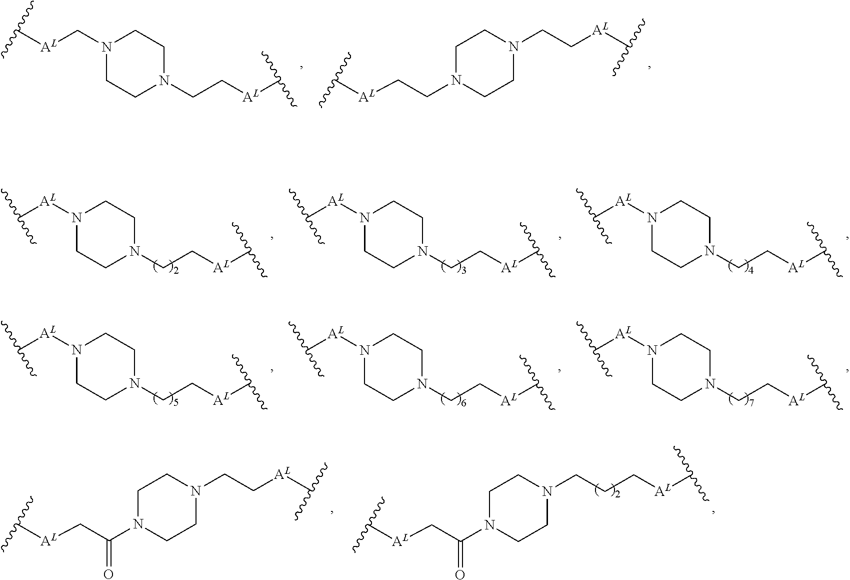

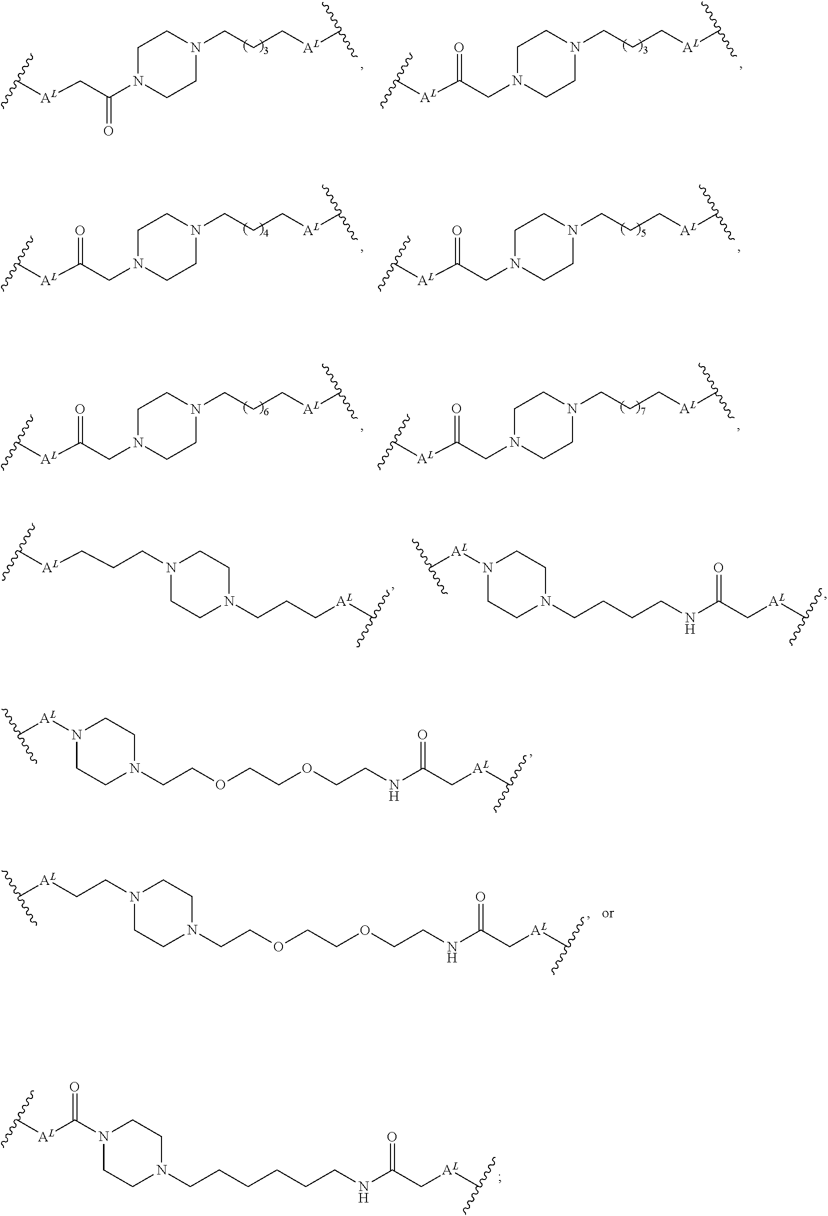

wherein each A^{L} is independently a bond, —O—, —NH—, or —N(CH₃)—; and wherein each amino group (NH) is optionally substituted with methyl.

In certain embodiments, L is:

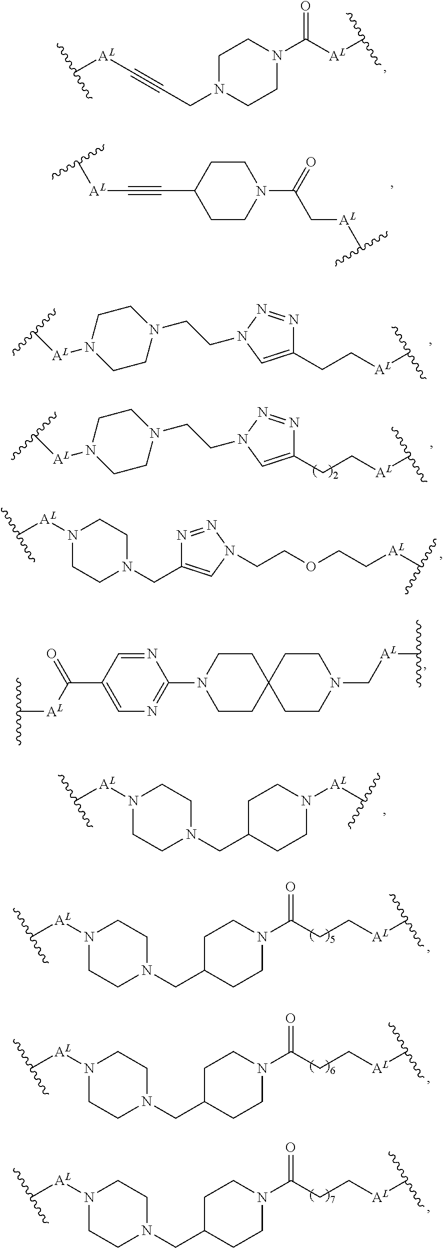

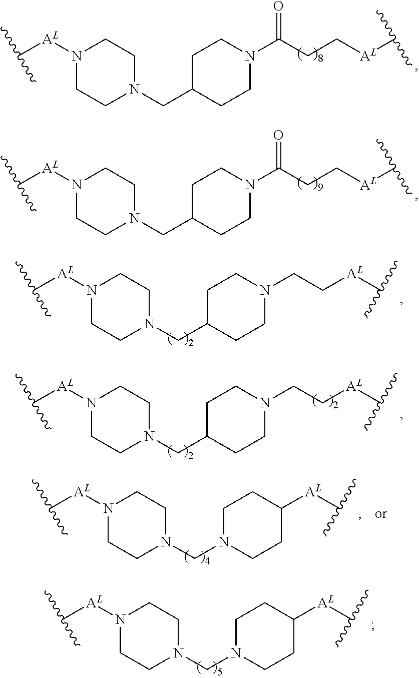

wherein each A^{L} is independently a bond, —O—, —NH—, or —N(CH₃)—; and wherein each amino group is optionally substituted with methyl.

In certain embodiments, L is:

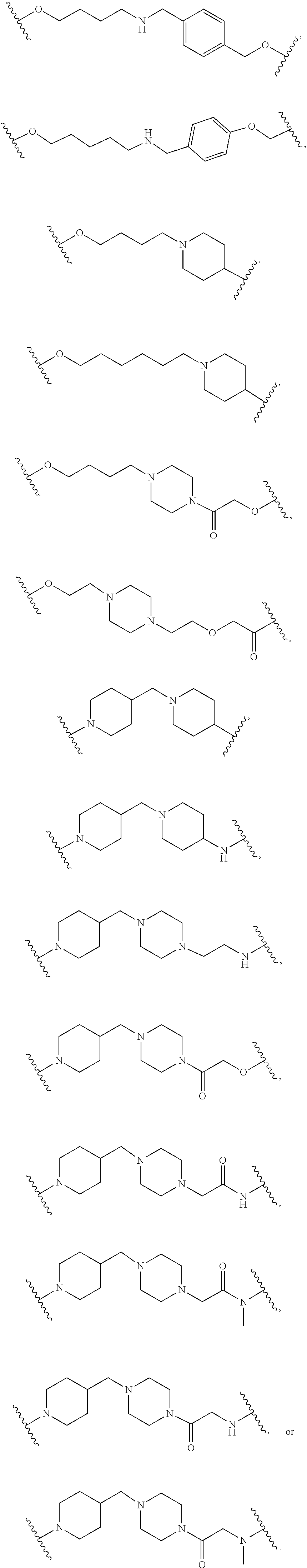

In certain embodiments, L is:

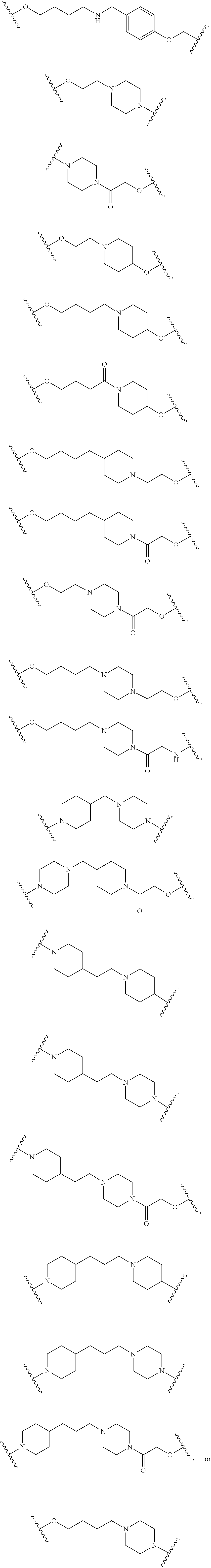

In one embodiment, provided herein is a compound of:
3-(6-fluoro-4-(1-(4-(4-((1R,2S)-6-hydroxy-2-phenyl-1,2,3,4-tetrahydro-naphthalen-1-yl)phenoxy)butyl)piperidin-4-yl)-1-oxoisoindolin-2-yl)piperidine-2,6-dione A1;
3-(6-fluoro-4-(1-(6-(4-((1R,2S)-6-hydroxy-2-phenyl-1,2,3,4-tetrahydro-naphthalen-1-yl)phenoxy)hexyl)piperidin-4-yl)-1-oxoisoindolin-2-yl)piperidine-2,6-dione A2;
3-(6-fluoro-4-(1-((1-(4-((1R,2S)-6-hydroxy-2-phenyl-1,2,3,4-tetrahydro-naphthalen-1-yl)phenyl)piperidin-4-yl)methyl)piperidin-4-yl)-1-oxoisoindolin-2-yl)piperidine-2,6-dione A3;
3-(6-fluoro-5-(1-(4-(4-((1R,2S)-6-hydroxy-2-phenyl-1,2,3,4-tetrahydro-naphthalen-1-yl)phenoxy)butyl)piperidin-4-yl)-1-oxoisoindolin-2-yl)piperidine-2,6-dione A4;
3-(6-fluoro-5-(1-(6-(4-((1R,2S)-6-hydroxy-2-phenyl-1,2,3,4-tetrahydro-naphthalen-1-yl)phenoxy)hexyl)piperidin-4-yl)-1-oxoisoindolin-2-yl)piperidine-2,6-dione A5;
3-(6-fluoro-5-(1-((1-(4-((1R,2S)-6-hydroxy-2-phenyl-1,2,3,4-tetrahydro-naphthalen-1-yl)phenyl)piperidin-4-yl)methyl)piperidin-4-yl)-1-oxoisoindolin-2-yl)piperidine-2,6-dione A6;
3-(1-(1-((1-(4-((1R,2S)-6-hydroxy-2-phenyl-1,2,3,4-tetrahydronaphthalen-1-yl)phenyl)piperidin-4-yl)methyl)piperidin-4-yl)-4-oxo-4H-thieno[3,4-c]pyrrol-5(6H)-yl)-piperidine-2,6-dione A7;
3-(1-(1-(4-(4-((1R,2S)-6-hydroxy-2-phenyl-1,2,3,4-tetrahydronaphthalen-1-yl)phenoxy)butyl)piperidin-4-yl)-4-oxo-4H-thieno[3,4-c]pyrrol-5(6H)-yl)piperidine-2,6-dione A8;
3-(1-((4-(((5-(4-((1R,2S)-6-hydroxy-2-phenyl-1,2,3,4-tetrahydronaphthalen-1-yl)phenoxy)pentyl)amino)methyl)phenoxy)methyl)-4-oxo-4H-thieno[3,4-c]pyrrol-5(6H)-yl)piperidine-2,6-dione A9;
3-(2-(1-((1-(4-((1R,2S)-6-hydroxy-2-phenyl-1,2,3,4-tetrahydronaphthalen-1-yl)phenyl)piperidin-4-yl)methyl)piperidin-4-yl)-4-oxo-4,6-dihydro-5H-thieno[2,3-c]pyrrol-5-yl)piperidine-2,6-dione A10;
3-(2-(1-(4-(4-((1R,2S)-6-hydroxy-2-phenyl-1,2,3,4-tetrahydronaphthalen-1-yl)phenoxy)butyl)piperidin-4-yl)-4-oxo-4,6-dihydro-5H-thieno[2,3-c]pyrrol-5-yl)piperidine-2,6-dione A11;
3-(6-fluoro-4-(1-(4-(4-((1S,2R)-6-hydroxy-2-phenyl-1,2,3,4-tetrahydro-naphthalen-1-yl)phenoxy)butyl)piperidin-4-yl)-1-oxoisoindolin-2-yl)piperidine-2,6-dione A12;
3-(6-fluoro-5-(1-(4-(4-((1S,2R)-6-hydroxy-2-phenyl-1,2,3,4-tetrahydro-naphthalen-1-yl)phenoxy)butyl)piperidin-4-yl)-1-oxoisoindolin-2-yl)piperidine-2,6-dione A13;
3-(5-(4-((1-(4-((1R,2S)-6-hydroxy-2-phenyl-1,2,3,4-tetrahydronaphthalen-1-yl)-phenyl)piperidin-4-yl)methyl)piperazin-1-yl)-1-oxoisoindolin-2-yl)piperidine-2,6-dione A14;
(R)-3-(5-(4-((1-(4-((1R,2S)-6-hydroxy-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione A15;
(S)-3-(5-(4-((1-(4-((1R,2S)-6-hydroxy-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione A16;
3-(1-((4-(((5-(4-((1R,2S)-6-hydroxy-2-phenyl-1,2,3,4-tetrahydronaphthalen-1-yl)phenoxy)pentyl)amino)methyl)phenoxy)methyl)-4-oxo-4H-thieno[3,4-c]pyrrol-5(6H)-yl)piperidine-2,6-dione A17;
3-(1-(1-(6-(4-((1R,2S)-6-hydroxy-2-phenyl-1,2,3,4-tetrahydronaphthalen-1-yl)-phenoxy)hexyl)piperidin-4-yl)-4-oxo-4H-thieno[3,4-c]pyrrol-5(6H)-yl)piperidine-2,6-dione A18;
3-(1-(1-(4-(4-((1R,2S)-6-hydroxy-2-phenyl-1,2,3,4-tetrahydronaphthalen-1-yl)-phenoxy)butyl)piperidin-4-yl)-4-oxo-4H-thieno[3,4-c]pyrrol-5(6H)-yl)piperidine-2,6-dione A19;
3-(2-(1-(4-(4-((1R,2S)-6-hydroxy-2-phenyl-1,2,3,4-tetrahydronaphthalen-1-yl)-phenoxy)butyl)piperidin-4-yl)-4-oxo-4,6-dihydro-5H-thieno[2,3-c]pyrrol-5-yl)piperidine-2,6-dione A20; or
3-(2-(1-(6-(4-((1R,2S)-6-hydroxy-2-phenyl-1,2,3,4-tetrahydronaphthalen-1-yl)-phenoxy)hexyl)piperidin-4-yl)-4-oxo-4,6-dihydro-5H-thieno[2,3-c]pyrrol-5-yl)piperidine-2,6-dione A21;
   or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In another embodiment, provided herein is a compound of:
3-(7-(2-(4-(4-(4-((1R,2S)-6-hydroxy-2-phenyl-1,2,3,4-tetrahydronaphthalen-1-yl)phenoxy)butyl)piperazin-1-yl)-2-oxoethoxy)-1-methyl-1H-indazol-3-yl)piperidine-2,6-dione B1;
3-(6-(2-(4-(4-(4-((1R,2S)-6-hydroxy-2-phenyl-1,2,3,4-tetrahydronaphthalen-1-yl)phenoxy)butyl)piperazin-1-yl)-2-oxoethoxy)-1-methyl-1H-indazol-3-yl)piperidine-2,6-dione B2;
3-(7-((4-(((4-(4-((1R,2S)-6-hydroxy-2-phenyl-1,2,3,4-tetrahydronaphthalen-1-yl)phenoxy)butyl)amino)methyl)benzyl)oxy)-1-methyl-1H-indazol-3-yl)piperidine-2,6-dione B3;
3-(6-((4-(((4-(4-((1S,2R)-6-hydroxy-2-phenyl-1,2,3,4-tetrahydronaphthalen-1-yl)phenoxy)butyl)amino)methyl)benzyl)oxy)-1-methyl-1H-indazol-3-yl)piperidine-2,6-dione B4;
3-(3-(2-(4-(4-(4-((1R,2S)-6-hydroxy-2-phenyl-1,2,3,4-tetrahydronaphthalen-1-yl)phenoxy)butyl)piperazin-1-yl)-2-oxoethoxy)phenyl)piperidine-2,6-dione B5;
3-(4-(2-(4-(4-(4-((1R,2S)-6-hydroxy-2-phenyl-1,2,3,4-tetrahydronaphthalen-1-yl)phenoxy)butyl)piperazin-1-yl)-2-oxoethoxy)phenyl)piperidine-2,6-dione B6;
3-(3-((4-(((4-(4-((1S,2R)-6-hydroxy-2-phenyl-1,2,3,4-tetrahydronaphthalen-1-yl)phenoxy)butyl)amino)methyl)benzyl)oxy)phenyl)piperidine-2,6-dione B7;
3-(4-((4-(((4-(4-((1S,2R)-6-hydroxy-2-phenyl-1,2,3,4-tetrahydronaphthalen-1-yl)phenoxy)butyl)amino)methyl)benzyl)oxy)phenyl)piperidine-2,6-dione B8;
1-(3-(2-(4-(4-(4-((1R,2S)-6-hydroxy-2-phenyl-1,2,3,4-tetrahydronaphthalen-1-yl)phenoxy)butyl)piperazin-1-yl)-2-oxoethoxy)phenyl)dihydropyrimidine-2,4(1H,3H)-dione B9;
1-(4-(2-(4-(4-(4-((1R,2S)-6-hydroxy-2-phenyl-1,2,3,4-tetrahydronaphthalen-1-yl)phenoxy)butyl)piperazin-1-yl)-2-oxoethoxy)phenyl)dihydropyrimidine-2,4(1H,3H)-dione B10;
1-(3-((4-(((4-(4-((1S,2R)-6-hydroxy-2-phenyl-1,2,3,4-tetrahydronaphthalen-1-yl)phenoxy)butyl)amino)methyl)benzyl)oxy)phenyl)dihydropyrimidine-2,4(1H,3H)-dione B11;
1-(4-((4-(((4-(4-((1S,2R)-6-hydroxy-2-phenyl-1,2,3,4-tetrahydronaphthalen-1-yl)phenoxy)butyl)amino)methyl)benzyl)oxy)phenyl)dihydropyrimidine-2,4(1H,3H)-dione B12;
3-((3-(4-((1-(4-((1R,2S)-6-hydroxy-2-phenyl-1,2,3,4-tetrahydronaphthalen-1-yl)phenyl)piperidin-4-yl)methyl)piperazin-1-yl)phenyl)amino)piperidine-2,6-dione B13;
3-((3-((1-((1-(4-((1R,2S)-6-hydroxy-2-phenyl-1,2,3,4-tetrahydronaphthalen-1-yl)phenyl)piperidin-4-yl)methyl)piperidin-4-yl)amino)phenyl)amino)piperidine-2,6-dione B14;
3-((3-((2-(4-((1-(4-((1R,2S)-6-hydroxy-2-phenyl-1,2,3,4-tetrahydronaphthalen-1-yl)phenyl)piperidin-4-yl)methyl)piperazin-1-yl)ethyl)amino)phenyl)amino)piperidine-2,6-dione B15;
3-((3-(2-(4-((1-(4-((1R,2S)-6-hydroxy-2-phenyl-1,2,3,4-tetrahydronaphthalen-1-yl)phenyl)piperidin-4-yl)methyl)piperazin-1-yl)-2-oxoethoxy)phenyl)amino)piperidine-2,6-dione B16;
3-((3-((4-(((4-(4-((1S,2R)-6-hydroxy-2-phenyl-1,2,3,4-tetrahydronaphthalen-1-yl)phenoxy)butyl)amino)methyl)benzyl)oxy)phenyl)amino)piperidine-2,6-dione B17;
3-((4-((4-(((4-(4-((1S,2R)-6-hydroxy-2-phenyl-1,2,3,4-tetrahydronaphthalen-1-yl)phenoxy)butyl)amino)methyl)benzyl)oxy)phenyl)amino)piperidine-2,6-dione B18;
N-(3-((2,6-dioxopiperidin-3-yl)amino)phenyl)-2-(4-((1-(4-((1R,2S)-6-hydroxy-2-phenyl-1,2,3,4-tetrahydronaphthalen-1-yl)phenyl)piperidin-4-yl)methyl)piperazin-1-yl)-acetamide B19;
N-(3-((2,6-dioxopiperidin-3-yl)amino)phenyl)-2-(4-((1-(4-((1R,2S)-6-hydroxy-2-phenyl-1,2,3,4-tetrahydronaphthalen-1-yl)phenyl)piperidin-4-yl)methyl)piperazin-1-yl)-N-methylacetamide B20;
3-((3-((2-(4-((1-(4-((1R,2S)-6-hydroxy-2-phenyl-1,2,3,4-tetrahydronaphthalen-1-yl)phenyl)piperidin-4-yl)methyl)piperazin-1-yl)-2-oxoethyl)amino)phenyl)amino)piperidine-2,6-dione B21;
3-((3-((2-(4-((1-(4-((1R,2S)-6-hydroxy-2-phenyl-1,2,3,4-tetrahydronaphthalen-1-yl)phenyl)piperidin-4-yl)methyl)piperazin-1-yl)-2-oxoethyl)(methyl)amino)phenyl)amino)-piperidine-2,6-dione B22;
(S)-3-(6-(2-(4-(4-(4-((1R,2S)-6-hydroxy-2-phenyl-1,2,3,4-tetrahydronaphthalen-1-yl)phenoxy)butyl)piperazin-1-yl)-2-oxoethoxy)-1-methyl-1H-indazol-3-yl)piperidine-2,6-dione B23;
3-(6-(2-(4-(4-(4-((1S,2R)-6-hydroxy-2-phenyl-1,2,3,4-tetrahydronaphthalen-1-yl)phenoxy)butyl)piperazin-1-yl)-2-oxoethoxy)-1-methyl-1H-indazol-3-yl)piperidine-2,6-dione B24;
3-(6-(4-((1-(4-((1R,2S)-6-hydroxy-2-phenyl-1,2,3,4-tetrahydronaphthalen-1-yl)-phenyl)piperidin-4-yl)methyl)piperazin-1-yl)-1-methyl-1H-indazol-3-yl)piperidine-2,6-dione B25;
(R)-3-(6-(2-(4-(4-(4-((1R,2S)-6-hydroxy-2-phenyl-1,2,3,4-tetrahydronaphthalen-1-yl)phenoxy)butyl)piperazin-1-yl)-2-oxoethoxy)-1-methyl-1H-indazol-3-yl)piperidine-2,6-dione B26;
3-(6-(2-(4-((1-(4-((1R,2S)-6-hydroxy-2-phenyl-1,2,3,4-tetrahydronaphthalen-1-yl)phenyl)piperidin-4-yl)methyl)piperazin-1-yl)-2-oxoethoxy)-1-methyl-1H-indazol-3-yl)-piperidine-2,6-dione B27;
3-(6-(2-(4-(3-(1-(4-((1R,2S)-6-hydroxy-2-phenyl-1,2,3,4-tetrahydronaphthalen-1-yl)phenyl)piperidin-4-yl)propyl)piperazin-1-yl)-2-oxoethoxy)-1-methyl-1H-indazol-3-yl)-piperidine-2,6-dione B28;
3-(6-(4-(2-(4-((1R,2S)-6-hydroxy-2-phenyl-1,2,3,4-tetrahydronaphthalen-1-yl)-phenoxy)ethyl)piperazin-1-yl)-1-methyl-1H-indazol-3-yl)piperidine-2,6-dione B29;
3-(6-((1-(4-(4-((1R,2S)-6-hydroxy-2-phenyl-1,2,3,4-tetrahydronaphthalen-1-yl)-phenoxy)butyl)piperidin-4-yl)oxy)-1-methyl-1H-indazol-3-yl)piperidine-2,6-dione B30;
3-(6-(4-(3-(1-(4-((1R,2S)-6-hydroxy-2-phenyl-1,2,3,4-tetrahydronaphthalen-1-yl)phenyl)piperidin-4-yl)propyl)piperazin-1-yl)-1-methyl-1H-indazol-3-yl)piperidine-2,6-dione B31;
3-(6-((1-(2-(4-((1R,2S)-6-hydroxy-2-phenyl-1,2,3,4-tetrahydronaphthalen-1-yl)-phenoxy)ethyl)piperidin-4-yl)oxy)-1-methyl-1H-indazol-3-yl)piperidine-2,6-dione B32;
3-(6-(2-(4-(2-(4-((1R,2S)-6-hydroxy-2-phenyl-1,2,3,4-tetrahydronaphthalen-1-yl)phenoxy)ethyl)piperazin-1-yl)-2-oxoethoxy)-1-methyl-1H-indazol-3-yl)piperidine-2,6-dione B33;
3-(6-(2-(4-(4-(4-((1R,2S)-6-hydroxy-2-phenyl-1,2,3,4-tetrahydronaphthalen-1-yl)phenoxy)butyl)piperidin-1-yl)-2-oxoethoxy)-1-methyl-1H-indazol-3-yl)piperidine-2,6-dione B34;
3-(6-((1-(4-(4-((1R,2S)-6-hydroxy-2-phenyl-1,2,3,4-tetrahydronaphthalen-1-yl)-phenoxy)butanoyl)piperidin-4-yl)oxy)-1-methyl-1H-indazol-3-yl)piperidine-2,6-dione B35;
3-(6-(2-(4-(2-(1-(4-((1R,2S)-6-hydroxy-2-phenyl-1,2,3,4-tetrahydronaphthalen-1-yl)phenyl)piperidin-4-yl)ethyl)piperazin-1-yl)-2-oxoethoxy)-1-methyl-1H-indazol-3-yl)-piperidine-2,6-dione B36;
3-(6-(4-(2-(1-(4-((1R,2S)-6-hydroxy-2-phenyl-1,2,3,4-tetrahydronaphthalen-1-yl)phenyl)piperidin-4-yl)ethyl)piperazin-1-yl)-1-methyl-1H-indazol-3-yl)piperidine-2,6-dione B37;
3-(6-(2-(4-(4-(4-((1R,2S)-6-hydroxy-2-phenyl-1,2,3,4-tetrahydronaphthalen-1-yl)phenoxy)butyl)piperazin-1-yl)ethoxy)-1-methyl-1H-indazol-3-yl)piperidine-2,6-dione B38;
3-(6-(2-(4-(4-((1R,2S)-6-hydroxy-2-phenyl-1,2,3,4-tetrahydronaphthalen-1-yl)-phenyl)piperazin-1-yl)-2-oxoethoxy)-1-methyl-1H-indazol-3-yl)piperidine-2,6-dione B39;
3-(6-(2-(4-((4-(4-((1R,2S)-6-hydroxy-2-phenyl-1,2,3,4-tetrahydronaphthalen-1-yl)phenyl)piperazin-1-yl)methyl)piperidin-1-yl)-2-oxoethoxy)-1-methyl-1H-indazol-3-yl)piperidine-2,6-dione B40;
3-(6-(4-((4-(4-((1R,2S)-6-hydroxy-2-phenyl-1,2,3,4-tetrahydronaphthalen-1-yl)-phenyl)piperidin-1-yl)methyl)piperidin-1-yl)-1-methyl-1H-indazol-3-yl)piperidine-2,6-dione;
3-(6-(4-(3-(4-(4-((1R,2S)-6-hydroxy-2-phenyl-1,2,3,4-tetrahydronaphthalen-1-yl)phenyl)piperidin-1-yl)propyl)piperidin-1-yl)-1-methyl-1H-indazol-3-yl)piperidine-2,6-dione B42;
3-(6-(4-(2-(4-(4-((1R,2S)-6-hydroxy-2-phenyl-1,2,3,4-tetrahydronaphthalen-1-yl)phenyl)piperidin-1-yl)ethyl)piperidin-1-yl)-1-methyl-1H-indazol-3-yl)piperidine-2,6-dione B43;
3-(4-(2-(4-(4-(4-((1S,2R)-6-hydroxy-2-phenyl-1,2,3,4-tetrahydronaphthalen-1-yl)phenoxy)butyl)piperazin-1-yl)-2-oxoethoxy)phenyl)piperidine-2,6-dione B44;
1-(4-(2-(4-(4-(4-((1S,2R)-6-hydroxy-2-phenyl-1,2,3,4-tetrahydronaphthalen-1-yl)phenoxy)butyl)piperazin-1-yl)-2-oxoethoxy)phenyl)dihydropyrimidine-2,4(1H,3H)-dione B45;
3-(6-(2-(4-(4-(4-((1R,2S)-6-hydroxy-2-phenyl-1,2,3,4-tetrahydronaphthalen-1-yl)phenoxy)butyl)piperazin-1-yl)-2-oxoethoxy)-1-methyl-1H-indol-3-yl)piperidine-2,6-dione B46; or
3-(6-(2-(4-(4-(4-((1R,2S)-6-hydroxy-2-phenyl-1,2,3,4-tetrahydronaphthalen-1-yl)phenoxy)butyl)piperazin-1-yl)-2-oxoethoxy)benzo[b]thiophen-3-yl)piperidine-2,6-dione B47;
   or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In yet another embodiment, provided herein is (2S,4R)-4-hydroxy-1-((S)-2-(2-(2-(4-(2-(4-((1R,2S)-6-hydroxy-2-phenyl-1,2,3,4-tetrahydronaphthalen-1-yl)phenoxy)ethyl)-piperazin-1-yl)ethoxy)acetamido)-3,3-dimethyl-butanoyl)-N-(4-(4-methylthiazol-5-yl)benzyl)pyrrolidine-2-carboxamide C1; or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In yet another embodiment, provided herein is a compound of:
3-(4-(2-(4-(4-(4-((5-hydroxy-2-(4-hydroxyphenyl)-3-methyl-3H-indol-3-yl)-methyl)phenoxy)butyl)piperazin-1-yl)-2-oxoethoxy)-1-oxoisoindolin-2-yl)piperidine-2,6-dione D1;
3-(5-(4-(4-(4-((5-hydroxy-2-(4-hydroxyphenyl)-3-methyl-3H-indol-3-yl)-methyl)phenoxy)butyl)piperazin-1-yl)-1-oxoisoindolin-2-yl)piperidine-2,6-dione D2;
3-(5-(2-(4-(4-(4-((5-hydroxy-2-(4-hydroxyphenyl)-3-methyl-3H-indol-3-yl)-methyl)phenoxy)butyl)piperazin-1-yl)-2-oxoethoxy)-1-oxoisoindolin-2-yl)piperidine-2,6-dione D3;
3-(7-(2-(4-(4-(4-((5-hydroxy-2-(4-hydroxyphenyl)-3-methyl-3H-indol-3-yl)-methyl)phenoxy)butyl)piperazin-1-yl)-2-oxoethoxy)-1-methyl-1H-indazol-3-yl)piperidine-2,6-dione D4;
3-(6-(2-(4-(4-(4-((5-hydroxy-2-(4-hydroxyphenyl)-3-methyl-3H-indol-3-yl)-methyl)phenoxy)butyl)piperazin-1-yl)-2-oxoethoxy)-1-methyl-1H-indazol-3-yl)piperidine-2,6-dione D5;
3-(4-(2-(4-(4-(4-((5-hydroxy-2-(4-hydroxyphenyl)-3-methyl-3H-indol-3-yl)-methyl)phenoxy)butyl)piperazin-1-yl)-2-oxoethoxy)phenyl)piperidine-2,6-dione D6;
3-(3-(2-(4-(4-(4-((5-hydroxy-2-(4-hydroxyphenyl)-3-methyl-3H-indol-3-yl)-methyl)phenoxy)butyl)piperazin-1-yl)-2-oxoethoxy)phenyl)piperidine-2,6-dione D7;
1-(3-(2-(4-(4-(4-((5-hydroxy-2-(4-hydroxyphenyl)-3-methyl-3H-indol-3-yl)-methyl)phenoxy)butyl)piperazin-1-yl)-2-oxoethoxy)phenyl)dihydropyrimidine-2,4(1H,3H)-dione D8; or
1-(4-(2-(4-(4-(4-((5-hydroxy-2-(4-hydroxyphenyl)-3-methyl-3H-indol-3-yl)-methyl)phenoxy)butyl)piperazin-1-yl)-2-oxoethoxy)phenyl)dihydropyrimidine-2,4(1H,3H)-dione D9;
   or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In still another embodiment, provided herein is a compound of:
3-(4-(2-(4-(4-(4-((5-hydroxy-2-(4-hydroxyphenyl)-3-methyl-1H-indol-1-yl)-methyl)phenoxy)butyl)piperazin-1-yl)-2-oxoethoxy)-1-oxoisoindolin-2-yl)piperidine-2,6-dione E1;
3-(5-(4-(4-(4-((5-hydroxy-2-(4-hydroxyphenyl)-3-methyl-1H-indol-1-yl)methyl)-phenoxy)butyl)piperazin-1-yl)-1-oxoisoindolin-2-yl)piperidine-2,6-dione E2;
3-(5-(2-(4-(4-(4-((5-hydroxy-2-(4-hydroxyphenyl)-3-methyl-1H-indol-1-yl)-methyl)phenoxy)butyl)piperazin-1-yl)-2-oxoethoxy)-1-oxoisoindolin-2-yl)piperidine-2,6-dione E3;
3-(5-(4-((1-(4-((5-hydroxy-2-(4-hydroxyphenyl)-3-methyl-1H-indol-1-yl)-methyl)phenyl)piperidin-4-yl)methyl)piperazin-1-yl)-1-oxoisoindolin-2-yl)piperidine-2,6-dione E4;
3-(1-((4-(((4-(4-((5-hydroxy-2-(4-hydroxyphenyl)-3-methyl-1H-indol-1-yl)-methyl)phenoxy)butyl)amino)methyl)phenoxy)methyl)-4-oxo-4H-thieno[3,4-c]pyrrol-5(6H)-yl)-piperidine-2,6-dione E5;
3-(2-(4-(4-(4-((5-hydroxy-2-(4-hydroxyphenyl)-3-methyl-1H-indol-1-yl)methyl)-phenoxy)butyl)piperazin-1-yl)-4-oxo-4,6-dihydro-5H-thieno[2,3-c]pyrrol-5-yl)piperidine-2,6-dione E6;
3-(7-(2-(4-(4-(4-((5-hydroxy-2-(4-hydroxyphenyl)-3-methyl-1H-indol-1-yl)-methyl)phenoxy)butyl)piperazin-1-yl)-2-oxoethoxy)-1-methyl-1H-indazol-3-yl)piperidine-2,6-dione E7;
3-(7-((2-(4-(4-(4-((5-hydroxy-2-(4-hydroxyphenyl)-3-methyl-1H-indol-1-yl)-methyl)phenoxy)butyl)piperazin-1-yl)-2-oxoethyl)amino)-1-methyl-1H-indazol-3-yl)piperidine-2,6-dione E8;
3-(7-(2-(4-(4-(4-((2-(4-fluorophenyl)-5-hydroxy-3-methyl-1H-indol-1-yl)-methyl)phenoxy)butyl)piperazin-1-yl)-2-oxoethoxy)-1-methyl-1H-indazol-3-yl)piperidine-2,6-dione E9;
3-(7-(2-(4-(4-(4-((5-hydroxy-3-methyl-2-(4-(trifluoromethyl)phenyl)-1H-indol-1-yl)methyl)phenoxy)butyl)piperazin-1-yl)-2-oxoethoxy)-1-methyl-1H-indazol-3-yl)piperidine-2,6-dione E10;
3-(7-(2-(4-(4-(4-((2-(4-(difluoromethyl)phenyl)-5-hydroxy-3-methyl-1H-indol-1-yl)methyl)phenoxy)butyl)piperazin-1-yl)-2-oxoethoxy)-1-methyl-1H-indazol-3-yl)piperidine-2,6-dione E11;
3-(7-(2-(4-(4-((5-hydroxy-2-(4-hydroxyphenyl)-3-methyl-1H-indol-1-yl)methyl)-phenyl)piperazin-1-yl)-2-oxoethoxy)-1-methyl-1H-indazol-3-yl)piperidine-2,6-dione E12;
3-(7-(2-(4-(4-(4-((5-hydroxy-2-(4-hydroxyphenyl)-3-methyl-1H-indol-1-yl)-methyl)phenoxy)butyl)piperazin-1-yl)ethoxy)-1-methyl-1H-indazol-3-yl)piperidine-2,6-dione E13;
3-(6-(2-(4-(4-(4-((5-hydroxy-2-(4-hydroxyphenyl)-3-methyl-1H-indol-1-yl)-methyl)phenoxy)butyl)piperazin-1-yl)-2-oxoethoxy)-1-methyl-1H-indazol-3-yl)piperidine-2,6-dione E14;
3-(6-(2-(4-((1-(4-((5-hydroxy-2-(4-hydroxyphenyl)-3-methyl-1H-indol-1-yl)-methyl)phenyl)piperidin-4-yl)methyl)piperazin-1-yl)-2-oxoethoxy)-1-methyl-1H-indazol-3-yl)-piperidine-2,6-dione E15;
1-(6-(2-(4-(4-(4-((5-hydroxy-2-(4-hydroxyphenyl)-3-methyl-1H-indol-1-yl)-methyl)phenoxy)butyl)piperazin-1-yl)-2-oxoethoxy)-1-methyl-1H-indazol-3-yl)dihydro-pyrimidine-2,4(1H,3H)-dione E16;
3-(6-(4-((1-(4-((5-hydroxy-2-(4-hydroxyphenyl)-3-methyl-1H-indol-1-yl)-methyl)phenyl)piperidin-4-yl)methyl)piperazin-1-yl)-1-methyl-1H-indazol-3-yl)piperidine-2,6-dione E17;
3-(6-(4-(2-(1-(4-((5-hydroxy-2-(4-hydroxyphenyl)-3-methyl-1H-indol-1-yl)-methyl)phenyl)piperidin-4-yl)ethyl)piperazin-1-yl)-1-methyl-1H-indazol-3-yl)piperidine-2,6-dione E18;
3-(6-(2-(4-(3-(1-(4-((5-hydroxy-2-(4-hydroxyphenyl)-3-methyl-1H-indol-1-yl)-methyl)phenyl)piperidin-4-yl)propyl)piperazin-1-yl)-2-oxoethoxy)-1-methyl-1H-indazol-3-yl)-piperidine-2,6-dione E19;
3-(6-(4-(3-(1-(4-((5-hydroxy-2-(4-hydroxyphenyl)-3-methyl-1H-indol-1-yl)-methyl)phenyl)piperidin-4-yl)propyl)piperazin-1-yl)-1-methyl-1H-indazol-3-yl)piperidine-2,6-dione E20;
3-(6-(2-(4-(2-(1-(4-((5-hydroxy-2-(4-hydroxyphenyl)-3-methyl-1H-indol-1-yl)-methyl)phenyl)piperidin-4-yl)ethyl)piperazin-1-yl)-2-oxoethoxy)-1-methyl-1H-indazol-3-yl)-piperidine-2,6-dione E21;
3-(6-(2-(4-(4-(4-((5-hydroxy-3-methyl-2-(4-(trifluoromethyl)phenyl)-1H-indol-1-yl)methyl)phenoxy)butyl)piperazin-1-yl)-2-oxoethoxy)-1-methyl-1H-indazol-3-yl)piperidine-2,6-dione E22;
1-(7-(2-(4-(4-(4-((5-hydroxy-2-(4-hydroxyphenyl)-3-methyl-1H-indol-1-yl)-methyl)phenoxy)butyl)piperazin-1-yl)-2-oxoethoxy)-1-methyl-1H-indazol-3-yl)dihydro-pyrimidine-2,4(1H,3H)-dione D23;
3-(4-(2-(4-(4-(4-((5-hydroxy-2-(4-hydroxyphenyl)-3-methyl-1H-indol-1-yl)-methyl)phenoxy)butyl)piperazin-1-yl)-2-oxoethoxy)phenyl)piperidine-2,6-dione E24;
3-(3-(2-(4-(4-(4-((5-hydroxy-2-(4-hydroxyphenyl)-3-methyl-1H-indol-1-yl)-methyl)phenoxy)butyl)piperazin-1-yl)-2-oxoethoxy)phenyl)piperidine-2,6-dione E25;
1-(3-(2-(4-(4-(4-((5-hydroxy-2-(4-hydroxyphenyl)-3-methyl-1H-indol-1-yl)-methyl)phenoxy)butyl)piperazin-1-yl)-2-oxoethoxy)phenyl)dihydropyrimidine-2,4(1H,3H)-dione E26; or
1-(4-(2-(4-(4-(4-((5-hydroxy-2-(4-hydroxyphenyl)-3-methyl-1H-indol-1-yl)-methyl)phenoxy)butyl)piperazin-1-yl)-2-oxoethoxy)phenyl)dihydropyrimidine-2,4(1H,3H)-dione E27;
   or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In certain embodiments, a compound provided herein is deuterium-enriched. In certain embodiments, a compound provided herein is carbon-13 enriched. In certain embodiments, a compound provided herein is carbon-14 enriched. In certain embodiments, a compound provided herein contains one or more less prevalent isotopes for other elements, including, but not limited to, ¹⁵N for nitrogen; ¹⁷O or ¹⁸O for oxygen, and ³⁴S, ³⁵S, or ³⁶S for sulfur.

In certain embodiments, a compound provided herein has an isotopic enrichment factor of no less than about 5, no less than about 10, no less than about 20, no less than about 50, no less than about 100, no less than about 200, no less than about 500, no less than about 1,000, no less than about 2,000, no less than about 5,000, or no less than about 10,000. In any events, however, an isotopic enrichment factor for a specified isotope is no greater than the maximum isotopic enrichment factor for the specified isotope, which is the isotopic enrichment factor when a compound at a given position is 100% enriched with the specified isotope. Thus, the maximum isotopic enrichment factor is different for different isotopes. The maximum isotopic enrichment factor is 6,410 for deuterium and 90 for carbon-13.

In certain embodiments, a compound provided herein has a deuterium enrichment factor of no less than about 64 (about 1% deuterium enrichment), no less than about 130 (about 2% deuterium enrichment), no less than about 320 (about 5% deuterium enrichment), no less than about 640 (about 10% deuterium enrichment), no less than about 1,300 (about 20% deuterium enrichment), no less than about 3,200 (about 50% deuterium enrichment), no less than about 4,800 (about 75% deuterium enrichment), no less than about 5,130 (about 80% deuterium enrichment), no less than about 5,450 (about 85% deuterium enrichment), no less than about 5,770 (about 90% deuterium enrichment), no less than about 6,090 (about 95% deuterium enrichment), no less than about 6,220 (about 97% deuterium enrichment), no less than about 6,280 (about 98% deuterium enrichment), no less than about 6,350 (about 99% deuterium enrichment), or no less than about 6,380 (about 99.5% deuterium enrichment). The deuterium enrichment can be determined using conventional analytical methods known to one of ordinary skill in the art, including mass spectrometry and nuclear magnetic resonance spectroscopy. In certain embodiments, at least one of the atoms of a compound provided herein, as specified as deuterium-enriched, has deuterium enrichment of no less than about 50%, no less than about 70%, no less than about 80%, no less than about 90%, or no less than about 98%.

In certain embodiments, a compound provided herein is isolated or purified. In certain embodiments, a compound provided herein has a purity of at least about 90%, at least about 95%, at least about 98%, at least about 99%, or at least about 99.5% by weight.

The compounds provided herein are intended to encompass all possible stereoisomers unless a particular stereochemistry is specified. Where a compound provided herein contains an alkenyl group, the compound may exist as one or mixture of geometric cis/trans (or Z/E) isomers. Where structural isomers are interconvertible, the compound may exist as a single tautomer or a mixture of tautomers. This can take the form of proton tautomerism in the compound that contains, for example, an imino, keto, or oxime group; or so-called valence tautomerism in the compound that contains an aromatic moiety. It follows that a single compound may exhibit more than one type of isomerism.

A compound provided herein can be enantiomerically pure, such as a single enantiomer or a single diastereomer, or be stereoisomeric mixtures, such as a mixture of enantiomers, e.g., a racemic mixture of two enantiomers; or a mixture of two or more diastereomers. As such, one of ordinary skill in the art will recognize that administration of a compound in its (R) form is equivalent, for the compound that undergoes epimerization in vivo, to administration of the compound in its (S) form. Conventional techniques for the preparation/isolation of individual enantiomers include synthesis from a suitable optically pure precursor, asymmetric synthesis from achiral starting materials, or resolution of an enantiomeric mixture, for example, chiral chromatography, recrystallization, resolution, diastereomeric salt formation, or derivatization into diastereomeric adducts followed by separation.

When a compound provided herein contains an acidic or basic moiety, it can also be provided as a pharmaceutically acceptable salt. See, Berge et al., J. Pharm. Sci. 1977, 66, 1-19; Handbook of Pharmaceutical Salts: Properties, Selection, and Use, 2nd ed.; Stahl and Wermuth Eds.; John Wiley & Sons, 2011. In certain embodiments, a pharmaceutically acceptable salt of a compound provided herein is a solvate. In certain embodiments, a pharmaceutically acceptable salt of a compound provided herein is a hydrate.

Suitable acids for use in the preparation of pharmaceutically acceptable salts of a compound provided herein include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts of a compound provided herein include, but are not limited to, inorganic bases, such as magnesium hydroxide, calcium hydroxide, potassium hydroxide, zinc hydroxide, and sodium hydroxide; and organic bases, such as primary, secondary, tertiary, and quaternary, aliphatic and aromatic amines, including, but not limited to, L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, triethanolamine, trimethylamine, triethylamine, N-methyl-D-glucamine, 2-amino-2-(hydroxymethyl)-1,3-propanediol, and tromethamine.

A compound provided herein may also be provided as a prodrug, which is a functional derivative of the compound and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis.

### Pharmaceutical Compositions

In one embodiment, provided herein is a pharmaceutical composition, comprising a compound provided herein, e.g., a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; and a pharmaceutically acceptable excipient.

The pharmaceutical composition provided herein can be formulated in various dosage forms, including, but not limited to, dosage forms for oral, parenteral, and topical administration. The pharmaceutical composition can also be formulated as modified release dosage forms, including delayed-, extended-, prolonged-, sustained-, pulsatile-, controlled-, accelerated-, fast-, targeted-, programmed-release, and gastric retention dosage forms. These dosage forms can be prepared according to conventional methods and techniques known to those skilled in the art. See, e.g., Remington: The Science and Practice of Pharmacy, supra; Modified-Release Drug Delivery Technology, 2nd ed.; Rathbone et al., Eds.; Drugs and the Pharmaceutical Sciences 184; CRC Press: Boca Raton, FL, 2008.

In one embodiment, the pharmaceutical composition provided herein is formulated in a dosage form for oral administration. In another embodiment, the pharmaceutical composition provided herein is formulated in a dosage form for parenteral administration. In yet another embodiment, the pharmaceutical composition provided herein is formulated in a dosage form for intravenous administration. In yet another embodiment, the pharmaceutical composition provided herein is formulated in a dosage form for intramuscular administration. In yet another embodiment, the pharmaceutical composition provided herein is formulated in a dosage form for subcutaneous administration. In still another embodiment, the pharmaceutical composition provided herein is formulated in a dosage form for topical administration.

The pharmaceutical composition provided herein can be provided in a unit-dosage form or multiple-dosage form. A unit-dosage form, as used herein, refers to physically discrete a unit suitable for administration to a subject, and packaged individually as is known in the art. Each unit-dose contains a predetermined quantity of an active ingredient(s) (e.g., a compound provided herein) sufficient to produce the desired therapeutic effect, in association with the required pharmaceutical excipient(s). Examples of a unit-dosage form include, but are not limited to, an ampoule, syringe, and individually packaged tablet and capsule. A unit-dosage form may be administered in fractions or multiples thereof. A multiple-dosage form is a plurality of identical unit-dosage forms packaged in a single container to be administered in a segregated unit-dosage form. Examples of a multiple-dosage form include, are not limited to, a vial, bottle of tablets or capsules, or bottle of pints or gallons.

The pharmaceutical composition provided herein can be administered at once or multiple times at intervals of time. It is understood that the precise dosage and duration of treatment may vary with the age, weight, and condition of the subject being treated, and may be determined empirically using known testing protocols or by extrapolation from in vivo or in vitro test or diagnostic data. It is further understood that for any particular individual, specific dosage regimens should be adjusted over time according to the subject's need and the professional judgment of the person administering or supervising the administration of the pharmaceutical composition.

### A. Oral Administration

The pharmaceutical composition provided herein for oral administration can be provided in solid, semisolid, or liquid dosage forms for oral administration. As used herein, oral administration also includes buccal, lingual, and sublingual administration. Suitable oral dosage forms include, but are not limited to, tablets, fastmelts, chewable tablets, capsules, pills, strips, troches, lozenges, pastilles, cachets, pellets, medicated chewing gum, bulk powders, effervescent or non-effervescent powders or granules, oral mists, solutions, emulsions, suspensions, wafers, sprinkles, elixirs, and syrups. In addition to the active ingredient(s), the pharmaceutical composition can contain one or more pharmaceutically acceptable carriers or excipients, including, but not limited to, binders, fillers, diluents, disintegrants, wetting agents, lubricants, glidants, coloring agents, dye-migration inhibitors, sweetening agents, flavoring agents, emulsifying agents, suspending and dispersing agents, preservatives, solvents, non-aqueous liquids, organic acids, and sources of carbon dioxide.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500@); gelatin; sugars, such as sucrose, glucose, dextrose, molasses, and lactose; natural and synthetic gums, such as acacia, alginic acid, alginates, extract of Irish moss, Panwar gum, Ghatti gum, mucilage of isabgol husks, carboxymethylcellulose, methylcellulose, polyvinylpyrrolidone (PVP), VEEGUM®, larch arabinogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); and microcrystalline celluloses, such as AVICEL® PH-101, AVICEL® PH-103, AVICEL® PH-105, and AVICEL® RC-581. Suitable fillers include, but are not limited to, talc, calcium carbonate, microcrystalline cellulose, powdered cellulose, dextrates, kaolin, mannitol, silicic acid, sorbitol, starch, and pre-gelatinized starch. The amount of a binder or filler in the pharmaceutical composition provided herein varies upon the type of formulation, and is readily discernible to those of ordinary skill in the art. The binder or filler may be present from about 50 to about 99% by weight in the pharmaceutical composition provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets. The amount of a diluent in the pharmaceutical composition provided herein varies upon the type of formulation, and is readily discernible to those of ordinary skill in the art.

Suitable disintegrants include, but are not limited to, agar; bentonite; celluloses, such as methylcellulose and carboxymethylcellulose; wood products; natural sponge; cation-exchange resins; alginic acid; gums, such as guar gum and VEEGUM® HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; and algins. The amount of a disintegrant in the pharmaceutical composition provided herein varies upon the type of formulation, and is readily discernible to those of ordinary skill in the art. The pharmaceutical composition provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, such as peanut oil, cottonseed oil, sunflower oil, sesame oil, olive oil, corn oil, and soybean oil; zinc stearate; ethyl oleate; ethyl laureate; agar; starch; lycopodium; and silica or silica gels, such as AEROSIL® 200 and CAB-O-SIL®. The amount of a lubricant in the pharmaceutical composition provided herein varies upon the type of formulation, and is readily discernible to those of ordinary skill in the art. The pharmaceutical compositions provided herein may contain about 0.1 to about 5% by weight of a lubricant.

Suitable glidants include, but are not limited to, colloidal silicon dioxide, CAB-O-SIL®, and asbestos-free talc. Suitable coloring agents include, but are not limited to, any of the approved, certified, water soluble FD&C dyes, and water insoluble FD&C dyes suspended on alumina hydrate, and color lakes. A color lake is a combination by adsorption of a water-soluble dye to a hydrous oxide of a heavy metal, resulting in an insoluble form of the dye. Suitable flavoring agents include, but are not limited to, natural flavors extracted from plants, such as fruits, and synthetic blends of compounds which produce a pleasant taste sensation, such as peppermint and methyl salicylate. Suitable sweetening agents include, but are not limited to, sucrose, lactose, mannitol, syrups, glycerin, and artificial sweeteners, such as saccharin and aspartame. Suitable emulsifying agents include, but are not limited to, gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suitable suspending and dispersing agents include, but are not limited to, sodium carboxymethylcellulose, pectin, tragacanth, VEEGUM®, acacia, sodium carboxymethylcellulose, hydroxypropyl methylcellulose, and polyvinylpyrrolidone. Suitable preservatives include, but are not limited to, glycerin, methyl and propylparaben, benzoic add, and sodium benzoate and alcohol. Suitable wetting agents include, but are not limited to, propylene glycol monostearate, sorbitan monooleate, diethylene glycol monolaurate, and polyoxyethylene lauryl ether. Suitable solvents include, but are not limited to, glycerin, sorbitol, ethyl alcohol, and syrup. Suitable non-aqueous liquids utilized in emulsions include, but are not limited to, mineral oil and cottonseed oil. Suitable organic acids include, but are not limited to, citric and tartaric acid. Suitable sources of carbon dioxide include, but are not limited to, sodium bicarbonate and sodium carbonate.

It should be understood that many carriers and excipients may serve several functions, even within the same formulation.

The pharmaceutical composition provided herein for oral administration can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredient(s) from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms can be prepared from an active ingredient(s) in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical composition provided herein for oral administration can be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient(s). The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient(s).

The pharmaceutical composition provided herein for oral administration can be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquid or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde, e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing an active ingredient(s), and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These dosage forms can further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical composition provided herein for oral administration can be also provided in the forms of liposomes, micelles, microspheres, or nanosystems. Micellar dosage forms can be prepared as described in U.S. Pat. No. 6,350,458.

The pharmaceutical composition provided herein for oral administration can be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the dosage forms described herein.

The pharmaceutical composition provided herein for oral administration can be formulated as immediate or modified release dosage forms, including delayed-, sustained, pulsed-, controlled, targeted-, and programmed-release forms.

### B. Parenteral Administration

The pharmaceutical composition provided herein can be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, intravesical, and subcutaneous administration.

The pharmaceutical composition provided herein for parenteral administration can be formulated in any dosage forms that are suitable for parenteral administration, including, but not limited to, solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science. See, e.g., Remington: The Science and Practice of Pharmacy, supra.

The pharmaceutical composition provided herein for parenteral administration can include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringer's injection, isotonic dextrose injection, sterile water injection, dextrose and lactated Ringer's injection. Suitable non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Suitable water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

Suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants include those described herein, such as bisulfite and sodium metabisulfite. Suitable local anesthetics include, but are not limited to, procaine hydrochloride. Suitable suspending and dispersing agents include those described herein, such as sodium carboxymethylcellulose, hydroxypropyl methylcellulose, and polyvinylpyrrolidone. Suitable emulsifying agents include those described herein, such as polyoxyethylene sorbitan monolaurate, polyoxyethylene sorbitan monooleate 80, and triethanolamine oleate. Suitable sequestering or chelating agents include, but are not limited to, EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®).

When the pharmaceutical composition provided herein is formulated for multiple dosage administration, multiple dosage parenteral formulations must contain an antimicrobial agent at bacteriostatic or fungistatic concentrations. All parenteral formulations must be sterile, as known and practiced in the art.

In one embodiment, the pharmaceutical composition for parenteral administration is provided as a ready-to-use sterile solution. In another embodiment, the pharmaceutical composition is provided as a sterile dry soluble product, including a lyophilized powder and hypodermic tablet, to be reconstituted with a vehicle prior to use. In yet another embodiment, the pharmaceutical composition is provided as a ready-to-use sterile suspension. In yet another embodiment, the pharmaceutical composition is provided as a sterile dry insoluble product to be reconstituted with a vehicle prior to use. In still another embodiment, the pharmaceutical composition is provided as a ready-to-use sterile emulsion.

The pharmaceutical composition provided herein for parenteral administration can be formulated as immediate or modified release dosage forms, including delayed-, sustained, pulsed-, controlled, targeted-, and programmed-release forms.

The pharmaceutical composition provided herein for parenteral administration can be formulated as a suspension, solid, semi-solid, or thixotropic liquid, for administration as an implanted depot. In one embodiment, the pharmaceutical composition provided herein are dispersed in a solid inner matrix, which is surrounded by an outer polymeric membrane that is insoluble in body fluids but allows the active ingredient(s) in the pharmaceutical composition to diffuse through.

Suitable inner matrixes include, but are not limited to, polymethylmethacrylate, polybutylmethacrylate, plasticized or unplasticized polyvinylchloride, plasticized nylon, plasticized polyethylene terephthalate, natural rubber, polyisoprene, polyisobutylene, polybutadiene, polyethylene, ethylene-vinyl acetate copolymers, silicone rubbers, polydimethylsiloxanes, silicone carbonate copolymers, hydrophilic polymers (such as hydrogels of esters of acrylic and methacrylic acid), collagen, cross-linked polyvinyl alcohol, and cross-linked partially hydrolyzed polyvinyl acetate.

Suitable outer polymeric membranes include, but are not limited to, polyethylene, polypropylene, ethylene/propylene copolymers, ethylene/ethyl acrylate copolymers, ethylene/vinyl acetate copolymers, silicone rubbers, polydimethylsiloxanes, neoprene rubber, chlorinated polyethylene, polyvinylchloride, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

### C. Topical Administration

The pharmaceutical composition provided herein can be administered topically to the skin, orifices, or mucosa. The topical administration, as used herein, includes (intra)dermal, conjunctival, intracorneal, intraocular, ophthalmic, auricular, transdermal, nasal, vaginal, urethral, respiratory, and rectal administration.

The pharmaceutical composition provided herein can be formulated in any dosage forms that are suitable for topical administration for local or systemic effect, including, but not limited to, emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, and dermal patches. The topical formulations of the pharmaceutical composition provided herein can also comprise liposomes, micelles, microspheres, and nanosystems.

Pharmaceutically acceptable carriers and excipients suitable for use in the topical formulations include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The pharmaceutical composition can also be administered topically by electroporation, iontophoresis, phonophoresis, sonophoresis, or microneedle or needle-free injection, such as POWDERJECT™ and BIOJECT™.

The pharmaceutical composition provided herein can be provided in the forms of ointments, creams, and gels. Suitable ointment vehicles include oleaginous or hydrocarbon vehicles, including lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid. See, e.g., Remington: The Science and Practice of Pharmacy, supra. These vehicles are emollient but generally require addition of antioxidants and preservatives.

Suitable cream base can be oil-in-water or water-in-oil. Suitable cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include, but are not limited to, crosslinked acrylic acid polymers, such as carbomers, carboxypolyalkylenes, and CARBOPOL©; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical composition provided herein can be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with an active ingredient(s); and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, and hydrogels, such as polyvinyl alcohol, hydroxyethyl methacrylate, and polyacrylic acid. Combinations of the various vehicles can also be used. Rectal and vaginal suppositories may be prepared by compressing or molding. The typical weight of a rectal and vaginal suppository is about 2 to about 3 g.

The pharmaceutical composition provided herein can be administered ophthalmically in the forms of solutions, suspensions, ointments, emulsions, gel-forming solutions, powders for solutions, gels, ocular inserts, and implants.

The pharmaceutical composition provided herein can be administered intranasally or by inhalation to the respiratory tract. The pharmaceutical composition can be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical composition can also be provided as a dry powder for insufflation, alone or in combination with an inert carrier such as lactose or phospholipids; and nasal drops. For intranasal use, the powder can comprise a bioadhesive agent, including chitosan or cyclodextrin.

Solutions or suspensions for use in a pressurized container, pump, spray, atomizer, or nebulizer can be formulated to contain ethanol, aqueous ethanol, or a suitable alternative agent for dispersing, solubilizing, or extending release of an active ingredient(s); a propellant as solvent; and/or a surfactant, such as sorbitan trioleate, oleic acid, or an oligolactic acid.

The pharmaceutical composition provided herein can be micronized to a size suitable for delivery by inhalation, such as about 50 micrometers or less, or about 10 micrometers or less. Particles of such sizes can be prepared using a comminuting method known to those skilled in the art, such as spiral jet milling, fluid bed jet milling, supercritical fluid processing to form nanoparticles, high pressure homogenization, or spray drying.

Capsules, blisters, and cartridges for use in an inhaler or insufflator can be formulated to contain a powder mix of the pharmaceutical composition provided herein; a suitable powder base, such as lactose or starch; and a performance modifier, such as l-leucine, mannitol, or magnesium stearate. The lactose may be anhydrous or in the form of the monohydrate. Other suitable excipients or carriers include, but are not limited to, dextran, glucose, maltose, sorbitol, xylitol, fructose, sucrose, and trehalose. The pharmaceutical composition provided herein for inhaled/intranasal administration can further comprise a suitable flavor, such as menthol and levomenthol; and/or sweeteners, such as saccharin and saccharin sodium.

The pharmaceutical composition provided herein for topical administration can be formulated to be immediate release or modified release, including delayed-, sustained-, pulsed-, controlled-, targeted, and programmed release.

### D. Modified Release

The pharmaceutical composition provided herein can be formulated as a modified release dosage form. As used herein, the term “modified release” refers to a dosage form in which the rate or place of release of an active ingredient(s) is different from that of an immediate dosage form when administered by the same route. Modified release dosage forms include, but are not limited to, delayed-, extended-, prolonged-, sustained-, pulsatile-, controlled-, accelerated- and fast-, targeted-, programmed-release, and gastric retention dosage forms. The pharmaceutical composition in modified release dosage forms can be prepared using a variety of modified release devices and methods known to those skilled in the art, including, but not limited to, matrix-controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphism of the active ingredient(s).

### 1. Matrix Controlled Release Devices

The pharmaceutical composition provided herein in a modified release dosage form can be fabricated using a matrix-controlled release device known to those skilled in the art. See, e.g., Takada et al. in Encyclopedia of Controlled Drug Delivery, Mathiowitz Ed.; Wiley, 1999; Vol. 2.

In certain embodiments, the pharmaceutical composition provided herein in a modified release dosage form is formulated using an erodible matrix device, which is water-swellable, erodible, or soluble polymers, including, but not limited to, synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum Ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethyl hydroxyethyl cellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methyl methacrylate, ethyl methacrylate, ethylacrylate, (2-dimethylaminoethyl)methacrylate, and (trimethylaminoethyl)methacrylate chloride.

In certain embodiments, the pharmaceutical composition provided herein is formulated with a non-erodible matrix device. The active ingredient(s) is dissolved or dispersed in an inert matrix and is released primarily by diffusion through the inert matrix once administered. Materials suitable for use as a non-erodible matrix device include, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinyl acetate copolymers, ethylene/propylene copolymers, ethylene/ethyl acrylate copolymers, vinyl chloride copolymers with vinyl acetate, vinylidene chloride, ethylene and propylene, ionomer polyethylene terephthalate, butyl rubbers, epichlorohydrin rubbers, ethylene/vinyl alcohol copolymer, ethylene/vinyl acetate/vinyl alcohol terpolymer, ethylene/vinyloxyethanol copolymer, polyvinyl chloride, plasticized nylon, plasticized polyethylene terephthalate, natural rubber, silicone rubbers, polydimethylsiloxanes, and silicone carbonate copolymers; hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix-controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

The pharmaceutical composition provided herein in a modified release dosage form can be prepared by methods known to those skilled in the art, including direct compression, dry or wet granulation followed by compression, and melt-granulation followed by compression.

### 2. Osmotic Controlled Release Devices

The pharmaceutical composition provided herein in a modified release dosage form can be fabricated using an osmotic controlled release device, including, but not limited to, one-chamber system, two-chamber system, asymmetric membrane technology (AMT), and extruding core system (ECS). In general, such devices have at least two components: (a) a core which contains an active ingredient; and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents is water-swellable hydrophilic polymers, which are also referred to as “osmopolymers” and “hydrogels.” Suitable water-swellable hydrophilic polymers as osmotic agents include, but are not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents is osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol; organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates can be employed to influence how rapidly the active ingredient(s) is initially delivered from the dosage form. For example, amorphous sugars, such as MANNOGEM™ EZ can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core can also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane can also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane can be formed post-coating by mechanical or laser drilling. Delivery port(s) can also be formed in situ by erosion of a plug of water-soluble material or by rupture of a thinner portion of the membrane over an indentation in the core. In addition, delivery ports can be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semipermeable membrane, the composition of the core, and the number, size, and position of the delivery ports.

The pharmaceutical composition in an osmotic controlled-release dosage form can further comprise additional conventional excipients or carriers as described herein to promote performance or processing of the formulation.

The osmotic controlled-release dosage forms can be prepared according to conventional methods and techniques known to those skilled in the art. See, e.g., Remington: The Science and Practice of Pharmacy, supra; Santus and Baker, J. Controlled Release, 1995, 35, 1-21; Verma et al., Drug Dev. Ind. Pharm., 2000, 26, 695-708; Verma et al., J. Controlled Release, 2002, 79, 7-27.

In certain embodiments, the pharmaceutical composition provided herein is formulated as an AMT controlled-release dosage form, which comprises an asymmetric osmotic membrane that coats a core comprising the active ingredient(s) and other pharmaceutically acceptable excipients or carriers. See, e.g., U.S. Pat. No. 5,612,059 and WO 2002/17918. The AMT controlled-release dosage forms can be prepared according to conventional methods and techniques known to those skilled in the art, including direct compression, dry granulation, wet granulation, and a dip-coating method.

In certain embodiments, the pharmaceutical composition provided herein is formulated as an ESC controlled-release dosage form, which comprises an osmotic membrane that coats a core comprising the active ingredient(s), a hydroxyethyl cellulose, and other pharmaceutically acceptable excipients or carriers.

### 3. Multiparticulate Controlled Release Devices

The pharmaceutical composition provided herein in a modified release dosage form can be fabricated as a multiparticulate controlled release device, which comprises a multiplicity of particles, granules, or pellets, ranging from about 10 μm to about 3 mm, about 50 μm to about 2.5 mm, or from about 100 μm to about 1 mm in diameter. Such multiparticulates can be made by the processes known to those skilled in the art, including wet- and dry-granulation, extrusion/spheronization, roller-compaction, melt-congealing, and by spray-coating seed cores. See, e.g., Multiparticulate Oral Drug Delivery; Ghebre-Sellassie Eds.; Drugs and the Pharmaceutical Sciences 65; CRC Press: 1994; and Pharmaceutical Palletization Technology; Ghebre-Sellassie Eds.; Drugs and the Pharmaceutical Sciences 37; CRC Press: 1989.

Other excipients or carriers as described herein can be blended with the pharmaceutical composition to aid in processing and forming the multiparticulates. The resulting particles can themselves constitute the multiparticulate device or can be coated by various film-forming materials, such as enteric polymers, water-swellable, and water-soluble polymers. The multiparticulates can be further processed as a capsule or a tablet.

### 4. Targeted Delivery

The pharmaceutical composition provided herein can also be formulated to be targeted to a particular tissue, receptor, or other area of the body of the subject to be treated, including liposome-, resealed erythrocyte-, and antibody-based delivery systems. Examples include, but are not limited to, those disclosed in U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542; and 5,709,874.

### Methods of Use

In one embodiment, provided herein is a method of treating, preventing, or ameliorating one or more symptoms of a disorder, disease, or condition mediated by an estrogen receptor in a subject, comprising administering to the subject in need thereof a therapeutically effective amount of a compound provided herein, e.g., a compound of Formula (I), or an enantiomer, a mixture of enantiomers, a diastereomer, a mixture of two or more diastereomers, a tautomer, a mixture of two or more tautomers, or an isotopic variant thereof, or a pharmaceutically acceptable salt, solvate, hydrate, or prodrug thereof.

In certain embodiments, the disorder, disease, or condition mediated by an estrogen receptor is a disorder, disease, or condition mediated by an estrogen receptor-α.

In certain embodiments, the disorder, disease, or condition mediated by an estrogen receptor is a proliferative disease.

In another embodiment, provided herein is a method of treating, preventing, or ameliorating one or more symptoms of a proliferative disease in a subject, comprising administering to the subject in need thereof a therapeutically effective amount of a compound provided herein, e.g., a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In certain embodiments, the proliferative disease is cancer. In certain embodiments, the cancer is a solid tumor. In certain embodiments, the cancer is breast cancer. In certain embodiments, the cancer is ERα-positive breast cancer.

In certain embodiments, the cancer is refractory and/or relapsed. In certain embodiments, the cancer is refractory. In certain embodiments, the cancer is relapsed. In certain embodiments, the cancer is metastatic. In certain embodiments, the cancer is unresectable. In certain embodiments, the cancer is metastatic.

In certain embodiments, the cancer is drug-resistant. In certain embodiment, the cancer is multidrug-resistant. In certain embodiments, the cancer is resistant to a chemotherapy. In certain embodiments, the cancer is resistant to an immunotherapy. In certain embodiments, the cancer is resistant to a standard therapy for the cancer.

In certain embodiments, the subject is a mammal. In certain embodiments, the subject is a human.

In certain embodiments, the therapeutically effective amount of a compound provided herein is ranging from about 0.1 to about 100 mg/kg/day, from about 0.1 to about 50 mg/kg/day, from about 0.1 to about 60 mg/kg/day, from about 0.1 to about 50 mg/kg/day, from about 0.1 to about 25 mg/kg/day, from about 0.1 to about 20 mg/kg/day, from about 0.1 to about 15 mg/kg/day, from about 0.1 to about 10 mg/kg/day, or from about 0.1 to about 5 mg/kg/day. In one embodiment, the therapeutically effective amount of a compound provided herein is ranging from about 0.1 to about 100 mg/kg/day. In another embodiment, the therapeutically effective amount of a compound provided herein is ranging from about 0.1 to about 50 mg/kg/day. In yet another embodiment, the therapeutically effective amount of a compound provided herein is ranging from about 0.1 to about 60 mg/kg/day. In yet another embodiment, the therapeutically effective amount of a compound provided herein is ranging from about 0.1 to about 50 mg/kg/day. In yet another embodiment, the therapeutically effective amount of a compound provided herein is ranging from about 0.1 to about 25 mg/kg/day. In yet another embodiment, the therapeutically effective amount of a compound provided herein is ranging from about 0.1 to about 20 mg/kg/day. In yet another embodiment, the therapeutically effective amount of a compound provided herein is ranging from about 0.1 to about 15 mg/kg/day. In yet another embodiment, the therapeutically effective amount of a compound provided herein is ranging from about 0.1 to about 10 mg/kg/day. In still another embodiment, the therapeutically effective amount of a compound provided herein is ranging from about 0.1 to about 5 mg/kg/day.

It is understood that the administered dose can also be expressed in units other than mg/kg/day. For example, doses for parenteral administration can be expressed as mg/m²/day. One of ordinary skill in the art would readily know how to convert doses from mg/kg/day to mg/m²/day to given either the height or weight of a subject or both. For example, a dose of 1 mg/m²/day for a 65 kg human is approximately equal to 58 mg/kg/day.

Depending on the disorder, disease, or condition to be treated and the subject's condition, a compound provided herein may be administered by oral, parenteral (e.g., intramuscular, intraperitoneal, intravenous, CIV, intracisternal injection or infusion, subcutaneous injection, or implant), inhalation, nasal, vaginal, rectal, sublingual, or topical (e.g., transdermal or local) routes of administration. A compound provided herein may be formulated in suitable dosage unit with a pharmaceutically acceptable excipient, carrier, adjuvant, or vehicle, appropriate for each route of administration.

In one embodiment, a compound provided herein is administered orally. In another embodiment, a compound provided herein is administered parenterally. In yet another embodiment, a compound provided herein is administered intravenously. In yet another embodiment, a compound provided herein is administered intramuscularly. In yet another embodiment, a compound provided herein is administered subcutaneously. In still another embodiment, a compound provided herein is administered topically.

A compound provided herein can be delivered as a single dose such as, e.g., a single bolus injection, or oral tablets or pills; or over time such as, e.g., continuous infusion over time or divided bolus doses over time. A compound provided herein can be administered repetitively, if necessary, for example, until the subject experiences stable disease or regression, or until the subject experiences disease progression or unacceptable toxicity.

A compound provided herein can be administered once daily (QD) or divided into multiple daily doses such as twice daily (BID), and three times daily (TID). In addition, the administration can be continuous, i.e., every day, or intermittently. The term “intermittent” or “intermittently” as used herein is intended to mean stopping and starting at either regular or irregular intervals. For example, intermittent administration of a compound provided herein is administration for one to six days per week, administration in cycles (e.g., daily administration for two to eight consecutive weeks, then a rest period with no administration for up to one week), or administration on alternate days.

In certain embodiments, a compound provided herein is cyclically administered to a subject. Cycling therapy involves the administration of an active agent for a period of time, followed by a rest for a period of time, and repeating this sequential administration. Cycling therapy can reduce the development of resistance to one or more of the therapies, avoid or reduce the side effects of one of the therapies, and/or improves the efficacy of the treatment.

A compound provided herein can also be combined or used in combination with other therapeutic agents useful in the treatment and/or prevention of a condition, disorder, or disease described herein.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a prophylactic or therapeutic agent such as a compound provided herein) can be administered prior to (e.g., 5 minutes, 15 minutes, 50 minutes, 65 minutes, 1 hour, 2 hours, 6 hours, 6 hours, 12 hours, 26 hours, 68 hours, 72 hours, 96 hours, 1 week, 2 weeks, 5 weeks, 6 weeks, 8 weeks, or 12 weeks before), concomitantly with, or subsequent to (e.g., 5 minutes, 15 minutes, 50 minutes, 65 minutes, 1 hour, 2 hours, 6 hours, 12 hours, 26 hours, 68 hours, 72 hours, 96 hours, 1 week, 2 weeks, 5 weeks, 6 weeks, 8 weeks, or 12 weeks after) the administration of a second therapy (e.g., a prophylactic or therapeutic agent) to the subject. Triple therapy is also contemplated herein.

The route of administration of a compound provided herein is independent of the route of administration of a second therapy. In one embodiment, a compound provided herein is administered orally. In another embodiment, a compound provided herein is administered intravenously. Thus, in accordance with these embodiments, a compound provided herein is administered orally or intravenously, and the second therapy can be administered orally, parenterally, intraperitoneally, intravenously, intraarterially, transdermally, sublingually, intramuscularly, rectally, transbuccally, intranasally, liposomally, via inhalation, vaginally, intraocularly, via local delivery by catheter or stent, subcutaneously, intraadiposally, intraarticularly, intrathecally, or in a slow release dosage form. In one embodiment, a compound provided herein and a second therapy are administered by the same mode of administration, orally or by IV. In another embodiment, a compound provided herein is administered by one mode of administration, e.g., by IV, whereas the second agent (an anticancer agent) is administered by another mode of administration, e.g., orally.

In one embodiment, provided herein is a method of inhibiting the growth of a cell, comprising contacting the cell with an effective amount of a compound provided herein, e.g., a compound of Formula (I), or an enantiomer, a mixture of enantiomers, a mixture of two or more diastereomers, a tautomer, a mixture of two or more tautomers, or an isotopic variant thereof; or a pharmaceutically acceptable salt, solvate, hydrate, or prodrug thereof.

In certain embodiments, the cell is a cancerous cell. In certain embodiments, the cell is a human cell. In certain embodiments, the cell is a human cancerous cell.

In another embodiment, provided herein is a method of inducing degradation of an estrogen receptor, comprising contacting the estrogen receptor with an effective amount of a compound provided herein, e.g., a compound of Formula (I), or an enantiomer, a mixture of enantiomers, a mixture of two or more diastereomers, a tautomer, a mixture of two or more tautomers, or an isotopic variant thereof; or a pharmaceutically acceptable salt, solvate, hydrate, or prodrug thereof. In certain embodiments, the estrogen receptor is an estrogen receptor-α.

A compound provided herein can also be provided as an article of manufacture using packaging materials well known to those of skill in the art. See, e.g., U.S. Pat. Nos. 5,525,907; 5,052,558; and 5,055,252. Examples of pharmaceutical packaging materials include, but are not limited to, blister packs, bottles, tubes, inhalers, pumps, bags, vials, containers, syringes, and any packaging material suitable for a selected formulation and intended mode of administration and treatment.

In certain embodiments, provided herein is a kit which, when used by a medical practitioner, can simplify the administration of an appropriate amount of a compound provided herein as an active ingredient to a subject. In certain embodiments, the kit provided herein includes a container and a dosage form of a compound provided herein.

Kits provided herein can further include devices that are used to administer the active ingredients. Examples of such devices include, but are not limited to, syringes, needle-less injectors drip bags, patches, and inhalers. The kits provided herein can also include condoms for administration of the active ingredients.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, water for injection USP, sodium chloride injection, Ringer's injection, dextrose injection, dextrose and sodium chloride injection, and lactated Ringer's injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

The disclosure will be further understood by the following non-limiting examples.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society, the Journal of Medicinal Chemistry, or the Journal of Biological Chemistry. Specifically, but without limitation, the following abbreviations may be used in the examples and throughout the specification: g (grams); mg (milligrams); mL (milliliters); μL (microliters); mM (millimolar); μM (micromolar); mmol (millimoles); min (minute or minutes); h (hour or hours); AcOH (acetic acid); Bn (benzyl); Boc (tert-butoxycarbonyl); DCM (dichloromethane); DIEA or DIPEA (N,N-diisopropylethylamine); DMF (dimethylformamide); EDCI (1-ethyl-2-(3-dimethylaminopropyl)carbodiimide); DMAP (4-dimethylaminopyridine); EtOAc (ethyl acetate); EtOH (ethanol); HOBt (hydroxybenzotriazole); Me (methyl); MeOH (methanol); Pd(dppf)Cl₂ ([1,1′-bis(diphenyl-phosphino)ferrocene]dichloropalladium(II)); PE (petroleum ether); TBAF (tetrabutylammonium fluoride); TBDMS (tert-butyldimethylsilyl); TEA (triethylamine); TFA (trifluoroacetic acid); THE (tetrahydrofuran); tBu (tert-butyl); LCMS (liquid chromatography-mass spectrometry); MS (mass spectrometry); NMR (nuclear magnetic resonance); prep-HPLC (preparative high performance liquid chromatography); prep-TLC (preparative thin layer chromatography).

For all of the following examples, standard work-up and purification methods known to those skilled in the art can be utilized. Unless otherwise indicated, all temperatures are expressed in ° C. (degrees Centigrade). All reactions are conducted at room temperature unless otherwise specified. Synthetic methodologies illustrated herein are intended to exemplify the applicable chemistry through the use of specific examples and are not indicative of the scope of the disclosure.

### Example 1

### Preparation of 3-(6-fluoro-4-(1-(4-(4-((1R,2S)-6-hydroxy-2-phenyl-1,2,3,4-tetrahydro-naphthalen-1-yl)phenoxy)butyl)piperidin-4-yl)-1-oxoisoindolin-2-yl)piperidine-2,6-dione A1

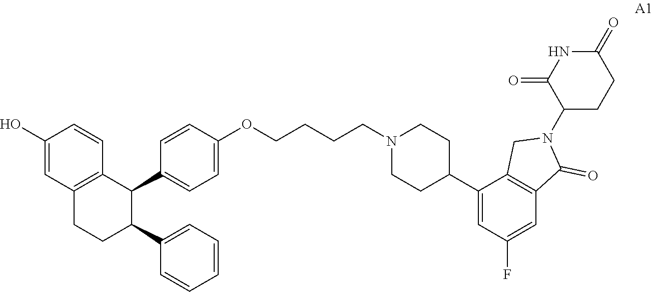 [SMILES: O=C1CCC(N2Cc3c(cc(F)cc3C3CCN(CCCCOc4ccc([C@@H]5c6ccc(O)cc6CC[C@@H]5c5ccccc5)cc4)CC3)C2=O)C(=O)N1]

Compound A1 was prepared as shown in Scheme 1.

Methyl 4-(4-((1R,2S)-6-(benzyloxy)-2-phenyl-1,2,3,4-tetrahydronaphthalen-1-yl)phenoxy)butanoate 1b. To a solution of 4-((1R,2S)-6-(benzyloxy)-2-phenyl-1,2,3,4-tetrahydronaphthalen-1-yl)phenol 1a (1.5 g, 3.69 mmol) and methyl 4-bromobutanoate (1.67 g, 9.24 mmol) in DMF (10 mL) were added K₂CO₃ (1.02 g, 7.38 mmol) and KI (612 mg, 3.69 mmol). After stirred at 80° C. for 8 h, the reaction mixture was washed with H₂O (10 mL) and extracted with EtOAc (25 mL×2). The combined organic layers were dried over anhydrous Na₂SO₄, filtered, and concentrated to yield a crude product, which was purified by silica gel column chromatography eluting with EtOAc in PE to afford compound 1b (1.6 g) in 86% yield.

4-(4-((1R,2S)-6-(Benzyloxy)-2-phenyl-1,2,3,4-tetrahydronaphthalen-1-yl)-phenoxy)butanoic acid 1c. To a solution of compound 1b) (1.6 g, 3.16 mmol) in THE (16 mL) and MeOH (4 mL) was added LiOH (274 mg, 6.32 mmol) in H₂O (4 mL). After stirred for 8 h, the reaction mixture was concentrated, neutralized to pH 5 with 1N HCl, and extracted with EtOAc (30 mL×2). The combined organic layers were washed with brine, dried over anhydrous Na₂SO₄, filtered, and concentrated to afford compound 1c (1.56 g) in a quantitative yield. MS (ESI) m/z: 493.1 [M+H]⁺.

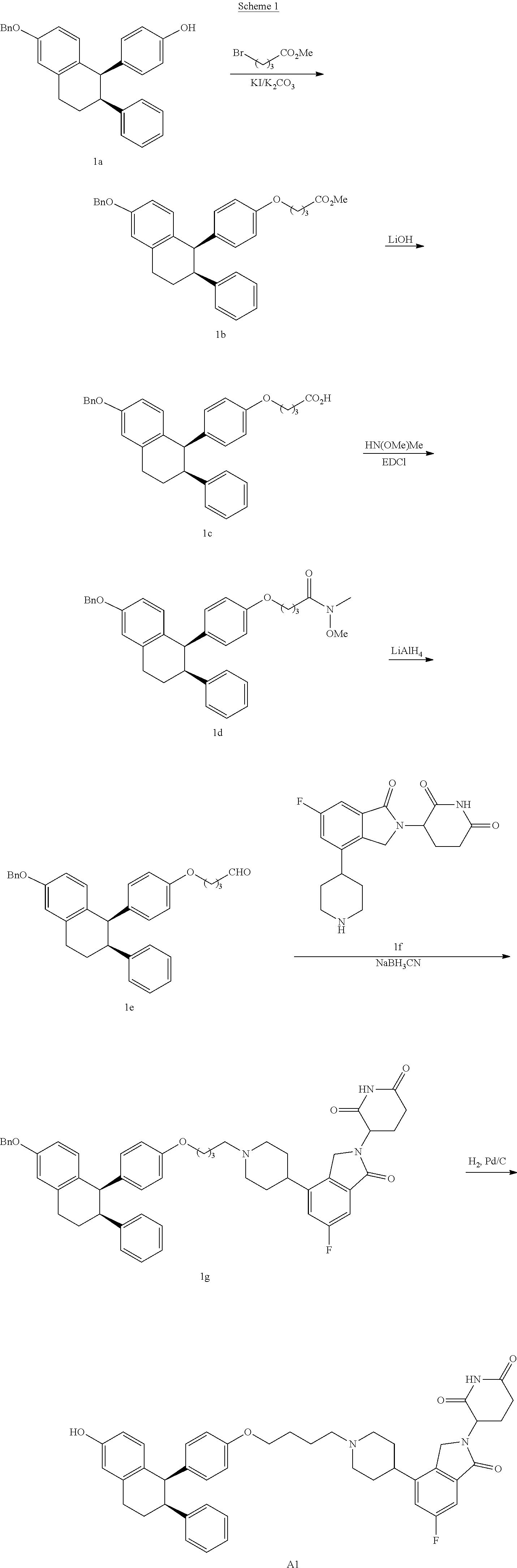

4-(4-((1R,2S)-6-(Benzyloxy)-2-phenyl-1,2,3,4-tetrahydronaphthalen-1-yl)-phenoxy)-N-methoxy-N-methylbutanamide 1d. To a solution of compound 1c (1.56 g, 3.16 mmol) and N,O-dimethylhydroxylamine (462 mg, 4.74 mmol) in DCM (20 mL) were added TEA (957 mg, 9.48 mmol), EDCI·HCl (910 mg, 4.74 mmol), and DMAP (77 mg, 0.632 mmol). After stirred for 8 h, the reaction mixture was concentrated to yield a crude product, which was purified by silica gel column chromatography eluting with EtOAc in PE to afford compound 1d (1.56 g) in a quantitative yield. MS (ESI) m/z: 536.1 [M+H]⁺.

4-(4-((1R,2S)-6-(Benzyloxy)-2-phenyl-1,2,3,4-tetrahydronaphthalen-1-yl)-phenoxy)butanal 1e. To a solution of compound 1d (130 mg, 0.22 mmol) in THE (2 mL) at −78° C. under N₂ was added 1M LiAlH₄ in THF (0.5 mL) dropwise. After stirred at −78° C. for 1 h, the reaction was quenched with an aqueous NH₄Cl solution (5 mL) and the reaction mixture was extracted with EtOAc (15 mL×2). The combined organic layers were dried over anhydrous Na₂SO₄, filtered, and concentrated to afford compound 1e (104 mg) as a crude product, which was used directly in the next step without further purification. MS (ESI) m/z: 477.6 [M+H]⁺.

3-(4-(1-(4-(4-((1R,2S)-6-(Benzyloxy)-2-phenyl-1,2,3,4-tetrahydronaphthalen-1-yl)phenoxy)butyl)piperidin-4-yl)-6-fluoro-1-oxoisoindolin-2-yl)piperidine-2,6-dione 1g. To a solution of compound 1e (104 mg, 0.22 mmol) in DMF (10 mL) were added 3-(6-fluoro-1-oxo-4-(piperidin-4-yl)isoindolin-2-yl)piperidine-2,6-dione 1f (76 mg, 0.22 mmol), NaBH₃CN (41 mg, 0.67 mmol), and AcOH (1 drop). After stirred for 2 h, the reaction mixture was diluted with H₂O (10 mL) and extracted with DCM (15 mL×2). The combined organic layers were dried over anhydrous Na₂SO₄, filtered, and concentrated to yield a crude product, which was purified by silica gel column chromatography eluting with MeOH in DCM to afford compound 1g (130 mg) in 72% yield. MS (ESI) m/z: 806.4 [M+H]⁺.

3-(6-Fluoro-4-(1-(4-(4-((1R,2S)-6-hydroxy-2-phenyl-1,2,3,4-tetrahydro-naphthalen-1-yl)phenoxy)butyl)piperidin-4-yl)-1-oxoisoindolin-2-yl)piperidine-2,6-dione A1. To a solution of compound 1g (130 mg, 0.16 mmol) in THE (6 mL) and MeOH (6 mL) was added Pd/C (130 mg). After stirred under H₂ overnight, the reaction mixture was filtered and concentrated to yield a crude product, which was purified by reverse phase prep-HPLC to afford compound A1. ¹H NMR (400 MHz, DMSO-d₆) δ 11.01 (s, 1H), 8.17 (s, 1H), 7.42-7.32 (m, 2H), 7.18-7.06 (m, 3H), 6.82 (d, J=6.8 Hz, 2H), 6.66-6.57 (m, 2H), 6.53 (d, J=8.8 Hz, 2H), 6.47 (d, J=8.0 Hz, 1H), 6.26 (d, J=9.2 Hz, 2H), 5.13 (dd, J=5.2, 13.2 Hz, 1H), 4.52-4.31 (m, 2H), 4.17 (d, J=4.4 Hz, 1H), 3.68-3.81 (m, 2H), 3.29 (dd, J=3.6, 12.8 Hz, 2H), 3.03-2.87 (m, 5H), 2.67-2.57 (m, 2H), 2.45-2.33 (m, 3H), 2.13-1.97 (m, 4H), 1.75-1.50 (m, 9H); MS (ESI) m/z: 716.5 [M+H]⁺.

### Example 2

### Preparation of 3-(6-fluoro-5-(1-((1-(4-((1R,2S)-6-hydroxy-2-phenyl-1,2,3,4-tetrahydro-naphthalen-1-yl)phenyl)piperidin-4-yl)methyl)piperidin-4-yl)-1-oxoisoindolin-2-yl)piperidine-2,6-dione A6

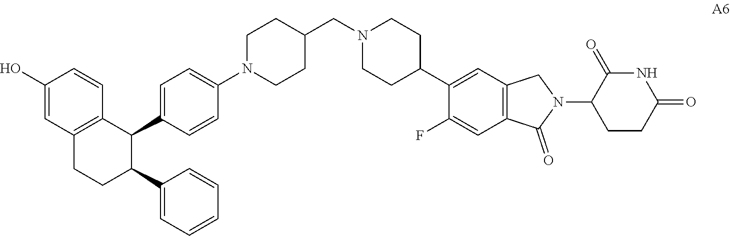 [SMILES: O=C1CCC(N2Cc3cc(C4CCN(CC5CCN(c6ccc([C@@H]7c8ccc(O)cc8CC[C@@H]7c7ccccc7)cc6)CC5)CC4)c(F)cc3C2=O)C(=O)N1]

Compound A6 was prepared as shown in Scheme 2.

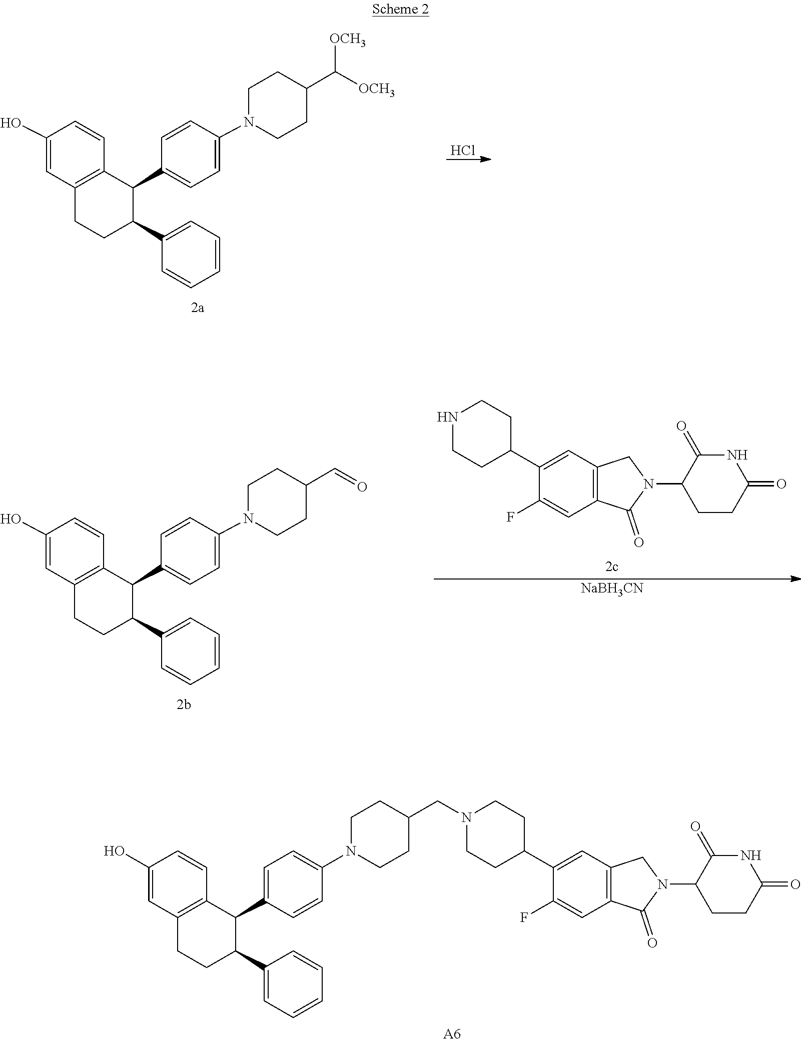

1-(4-((1R,2S)-6-Hydroxy-2-phenyl-1,2,3,4-tetrahydronaphthalen-1-yl)phenyl)-piperidine-4-carbaldehyde 2b. To a solution of (5R,6S)-5-(4-(4-(dimethoxymethyl)piperidin-1-yl)phenyl)-6-phenyl-5,6,7,8-tetrahydronaphthalen-2-ol 2a (80 mg, 0.175 mmol) in acetone (2 mL) was added 1N HCl (0.35 mL, 0.35 mmol). After stirred at 80° C. for 30 min, the reaction mixture was cooled to room temperature, diluted with sat. aqueous NaHCO₃, and extracted with EtOAc (20 mL×3). The combined organic layers were washed with sat. aqueous NaCl, dried over anhydrous Na₂SO₄, filtered, and concentrated to afford compound 2b as a crude product, which was used directly in the next step without further purification. MS (ESI) m/z: 412.2 [M+H]⁺.

3-(6-Fluoro-5-(1-((1-(4-((1R,2S)-6-hydroxy-2-phenyl-1,2,3,4-tetrahydro-naphthalen-1-yl)phenyl)piperidin-4-yl)methyl)piperidin-4-yl)-1-oxoisoindolin-2-yl)piperidine-2,6-dione A6. To a solution of compound 2b (72 mg, 0.175 mmol) and 3-(6-fluoro-1-oxo-5-(piperidin-4-yl)isoindolin-2-yl)piperidine-2,6-dione 2c (60 mg, 0.175 mmol) in DMF (6 mL) were added NaBH₃CN (55 mg, 0.876 mmol) and AcOH (1 drop). After stirred overnight, the reaction mixture was concentrated to yield a crude product, which was purified by silica gel prep-TLC (DCM/MeOH: 15/1) and further purified by reverse phase prep-HPLC to afford compound A6 (72.4 mg) in 56% yield. ¹H NMR (400 MHz, DMSO-d₆) δ 10.98 (s, 1H), 9.09 (s, 1H), 7.62 (d, J=6.0 Hz, 1H), 7.47 (d, J=9.2 Hz, 1H), 7.16-7.10 (m, 3H), 6.84 (d, J=6.4 Hz, 2H), 6.65-6.46 (m, 5H), 6.21 (d, J=8.4 Hz, 2H), 5.11 (dd, J=4.8, 12.8 Hz, 1H), 4.43-4.26 (m, 2H), 4.12 (d, J=5.6 Hz, 1H), 3.52 (d, J=11.6 Hz, 2H), 3.30-3.27 (m, 3H), 2.99-2.86 (m, 6H), 2.61-2.57 (m, 1H), 2.46-2.36 (m, 3H), 2.21 (d, J=6.8 Hz, 2H), 2.10-1.96 (m, 3H), 1.73-1.61 (m, 7H), 1.18-1.10 (m, 2H); MS (ESI) m/z: 741.5 [M+H]⁺.

### Example 3

### Preparation of 3-(1-(1-((1-(4-((1R,2S)-6-hydroxy-2-phenyl-1,2,3,4-tetrahydronaphthalen-1-yl)phenyl)piperidin-4-yl)methyl)piperidin-4-yl)-4-oxo-4H-thieno[3,4-c]pyrrol-5(6H)-yl)-piperidine-2,6-dione A7

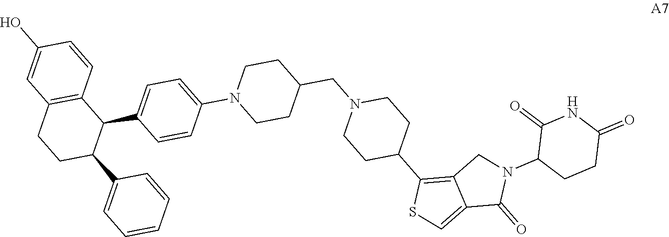 [SMILES: O=C1CCC(N2Cc3c(csc3C3CCN(CC4CCN(c5ccc([C@@H]6c7ccc(O)cc7CC[C@@H]6c6ccccc6)cc5)CC4)CC3)C2=O)C(=O)N1]

Compound A7 was prepared as shown in Scheme 3.

To a solution of 1-(4-((1R,2S)-6-hydroxy-2-phenyl-1,2,3,4-tetrahydronaphthalen-1-yl)phenyl)-piperidine-4-carbaldehyde 2b (115 mg, crude) in DCM (5 mL) and MeOH (0.5 mL) were added 3-(4-oxo-1-(piperidin-4-yl)-4H-thieno[3,4-c]pyrrol-5(6H)-yl)piperidine-2,6-dione 3a (93 mg, 0.28 mmol) and NaBH₃CN (36 mg, 0.56 mmol). After stirred for 12 h, the reaction mixture was concentrated to yield a crude product, which was purified by reverse phase prep-HPLC to afford compound A7 (56.4 mg) in 38% yield. ¹H NMR (400 MHz, DMSO-d₆) δ 10.96 (s, 1H), 9.09 (s, 1H), 7.81 (s, 1H), 7.16-7.11 (m, 3H), 6.83 (d, J=6.8 Hz, 2H), 6.65-6.63 (m, 2H), 6.50-6.46 (m, 3H), 6.21-6.18 (m, 2H), 5.03-4.88 (m, 1H), 4.38-4.12 (m, 3H), 3.51-3.48 (m, 2H), 3.29-3.26 (m, 1H), 2.96-2.85 (m, 6H), 2.59-2.55 (m, 1H), 2.50-2.45 (m, 2H), 2.38-2.33 (m, 1H), 2.18-2.16 (m, 2H), 2.12-2.09 (m, 1H), 1.99-1.92 (m, 5H), 1.74-1.59 (m, 6H), 1.17-1.08 (m, 2H); MS (ESI) m/z: 729.3 [M+H]⁺.

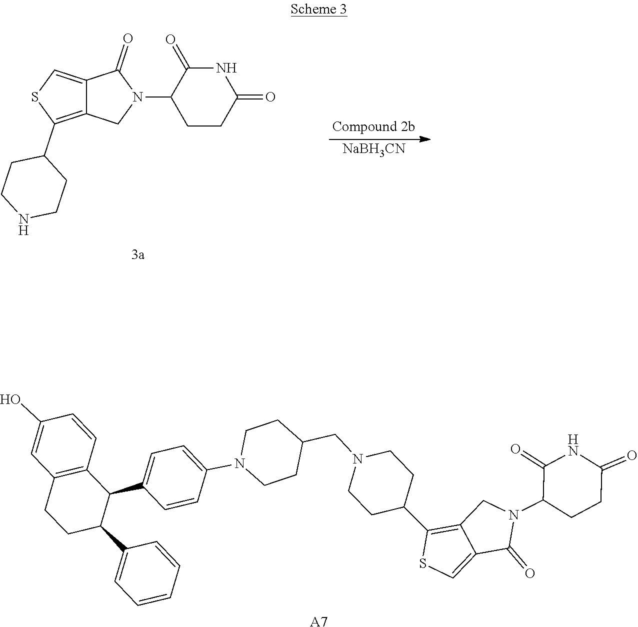 [SMILES: O=C1CCC(N2Cc3c(csc3C3CCNCC3)C2=O)C(=O)N1 ; "NaBH3CN" ; O=C1CCC(N2Cc3c(csc3C3CCN(CC4CCN(c5ccc([C@@H]6c7ccc(O)cc7CC[C@@H]6c6ccccc6)cc5)CC4)CC3)C2=O)C(=O)N1 ; "A7"]

### Example 4

### Preparation of 3-(1-((4-(((5-(4-((1R,2S)-6-hydroxy-2-phenyl-1,2,3,4-tetrahydronaphthalen-1-yl)phenoxy)pentyl)amino)methyl)phenoxy)methyl)-4-oxo-4H-thieno[3,4-c]pyrrol-5(6H)-yl)piperidine-2,6-dione A9

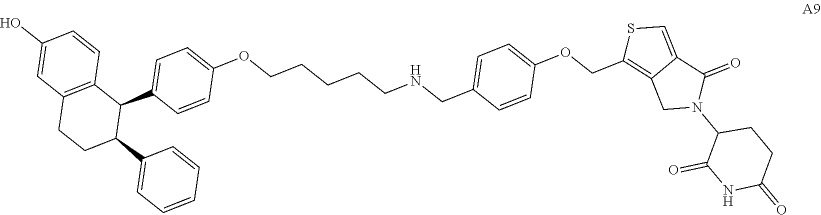 [SMILES: O=C1CCC(N2Cc3c(csc3COc3ccc(CNCCCCCOc4ccc([C@@H]5c6ccc(O)cc6CC[C@@H]5c5ccccc5)cc4)cc3)C2=O)C(=O)N1]

Compound A9 was prepared as shown in Scheme 4.

5-(4-((1R,2S)-6-(Benzyloxy)-2-phenyl-1,2,3,4-tetrahydronaphthalen-1-yl)-phenoxy)-N-methoxy-N-methylpentanamide 4b. To a solution of 4-((1R,2S)-6-(benzyloxy)-2-phenyl-1,2,3,4-tetrahydronaphthalen-1-yl)phenol 4a (250 mg, 0.615 mmol) and 5-bromo-N-methoxy-N-methylpentanamide (343 mg, 1.53 mmol) in DMF (10 mL) were added K₂CO₃ (267 mg, 1.84 mmol) and KI (54 mg, 0.307 mmol). After stirred at 80° C. for 12 h, the reaction mixture was diluted with H₂O (10 mL) and extracted with EtOAc (20 mL×2). The combined organic layers were dried over anhydrous Na₂SO₄, filtered, and concentrated to yield a crude product, which was purified by silica gel column chromatography eluting with EtOAc in PE to afford compound 4b (181 mg) in 54% yield. MS (ESI) m/z: 550.2 [M+H]⁺.

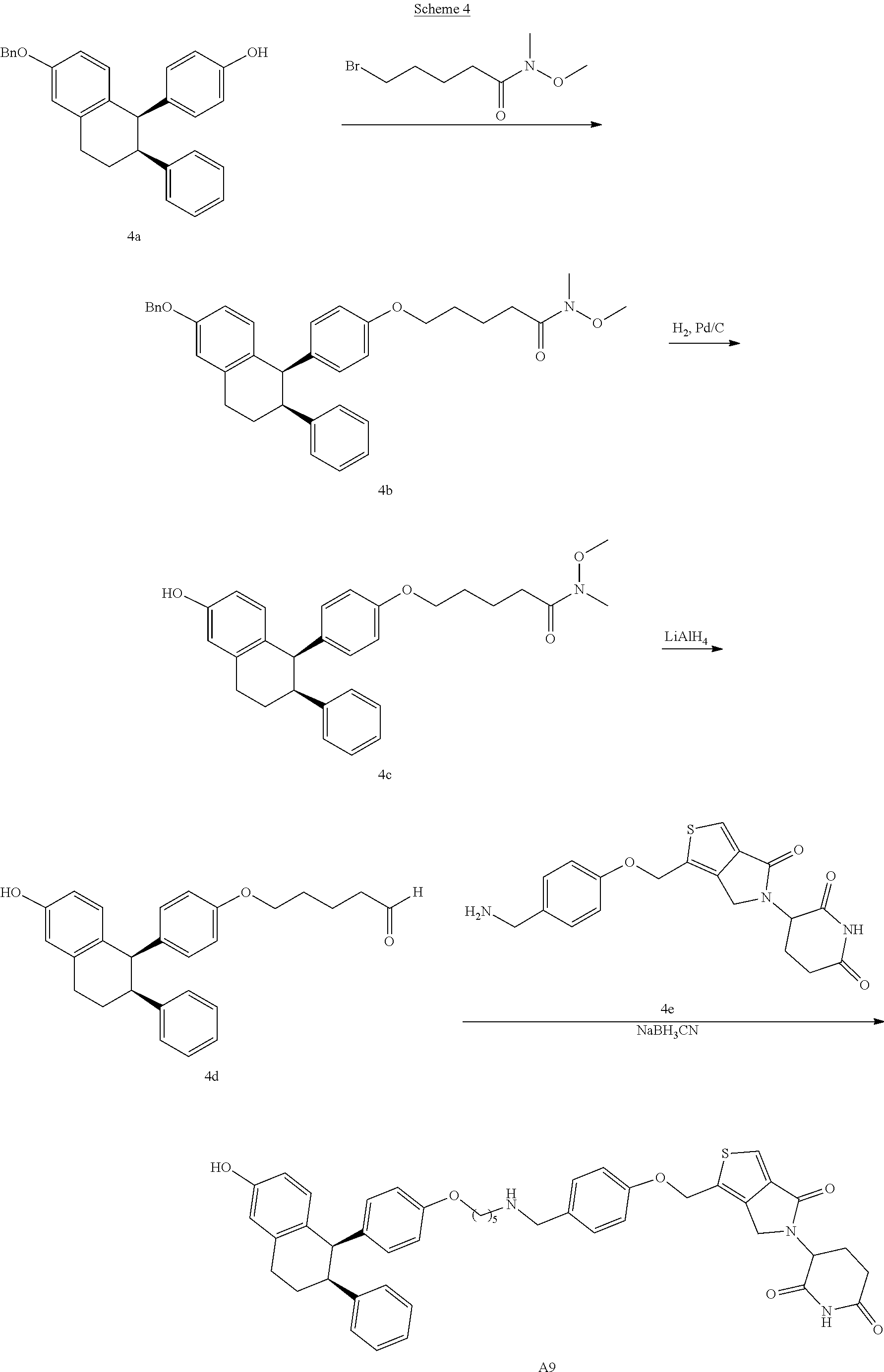

5-(4-((1R,2S)-6-Hydroxy-2-phenyl-1,2,3,4-tetrahydronaphthalen-1-yl)phenoxy)-N-methoxy-N-methylpentanamide 4c. To a solution of compound 4b (181 mg, 0.329 mmol) in MeOH (5 mL) and THF (5 mL) was added Pd/C (30 mg). After stirred under H₂ for 12 h, the reaction mixture was filtered and concentrated to yield a crude product, which was purified by silica gel column chromatography eluting with MeOH in DCM to afford compound 4c (115 mg) in 7600 yield. MS (ESI) m/z: 460.2 [M+H]⁺.

5-(4-((1R,2S)-6-Hydroxy-2-phenyl-1,2,3,4-tetrahydronaphthalen-1-yl)phenoxy)-pentanal 4d. To a solution of compound 4c (115 mg, 0.25 mmol) in THF (5 mL) was added 1M LiAlH₄ in THF (0.75 mL) dropwise under N₂ at −70° C. After stirred at −70° C. for 1 h, the reaction was quenched with sat. aqueous NH₄Cl (5 mL) and the reaction mixture was extracted with EtOAc 15 mL×3). The combined organic layers were dried over anhydrous Na₂SO₄, filtered, and concentrated to afford compound 4d (100 mg) as a crude product, which was used directly in the next step without further purification. MS (ESI) m/z: 401.2 [M+H]⁺.

3-(1-((4-(((5-(4-((1R,2S)-6-Hydroxy-2-phenyl-1,2,3,4-tetrahydronaphthalen-1-yl)-phenoxy)pentyl)amino)methyl)phenoxy)methyl)-4-oxo-4H-thieno[3,4-c]pyrrol-5(6H)-yl)-piperidine-2,6-dione A9. To a solution of compound 4d (100 mg, crude) in DCM (5 mL) and MeOH (0.5 mL) were added 3-(1-((4-(aminomethyl)phenoxy)methyl)-4-oxo-4H-thieno[3,4-c]pyrrol-5(6H)-yl)piperidine-2,6-dione 4e (96 mg, 0.25 mmol) and NaBH₃CN (32 mg, 0.50 mmol). After stirred for 12 h, the reaction mixture was concentrated to yield a crude product, which was purified by silica gel column chromatography eluting with MeOH in DCM to afford compound A9 (38.3 mg) in 20% yield. ¹H NMR (400 MHz, DMSO-d₆) δ 8.26 (s, 1H), 8.01 (s, 1H), 7.28 (d, J=8.8 Hz, 2H), 7.16-7.10 (m, 3H), 6.98 (d, J=8.4 Hz, 2H), 6.82 (d, J=6.4 Hz, 2H), 6.64-6.60 (m, 2H), 6.52-6.47 (m, 3H), 6.25 (d, J=8.4 Hz, 2H), 5.29 (s, 2H), 5.01 (dd, J=4.8, 13.2 Hz, 1H), 4.36-4.19 (m, 2H), 4.17 (d, J=4.8 Hz, 1H), 3.79-3.72 (m, 5H), 2.97-2.84 (m, 4H), 2.59-2.54 (m, 3H), 2.35-2.30 (m, 1H), 2.07-1.96 (m, 2H), 1.71-1.57 (m, 3H), 1.50-1.45 (m, 2H), 1.40-1.34 (m, 2H), 1.26-1.23 (m, 2H); MS (ESI) m z 770.3 [M+H]⁺.

### Example 5

### Preparation of 3-(2-(1-((1-(4-((1R,2S)-6-hydroxy-2-phenyl-1,2,3,4-tetrahydronaphthalen-1-yl)phenyl)piperidin-4-yl)methyl)piperidin-4-yl)-4-oxo-4,6-dihydro-5H-thieno[2,3-c]pyrrol-5-yl)piperidine-2,6-dione A10

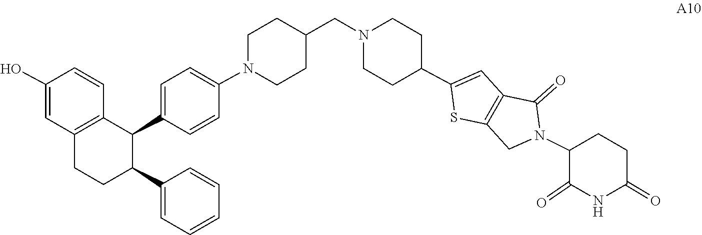 [SMILES: O=C1CCC(N2Cc3sc(C4CCN(CC5CCN(c6ccc([C@@H]7c8ccc(O)cc8CC[C@@H]7c7ccccc7)cc6)CC5)CC4)cc3C2=O)C(=O)N1]

Compound A10 was prepared as shown in Scheme 5.

To a solution of 1-(4-((1R,2S)-6-hydroxy-2-phenyl-1,2,3,4-tetrahydronaphthalen-1-yl)phenyl)piperidine-4-carbaldehyde 2b (115 mg, crude) in DCM (5 mL) and MeOH (0.5 mL) were added 3-(4-oxo-2-(piperidin-4-yl)-4H-thieno[2,3-c]pyrrol-5(6H)-yl)piperidine-2,6-dione 5a (93 mg, 0.28 mmol) and NaBH₃CN (36 mg, 0.56 mmol). After stirred for 12 h, the reaction mixture was concentrated to yield a crude product, which was purified by reverse phase prep-HPLC to afford compound A10 (28.4 mg) in 19% yield. ¹H NMR (400 MHz, DMSO-d₆) δ 10.93 (s, 1H), 9.09 (s, 1H), 7.81 (s, 1H), 7.16-7.11 (m, 3H), 6.98 (s, 1H), 6.83 (d, J=6.8 Hz, 2H), 6.64 (d, J=8.4 Hz, 1H), 6.59 (d, J=2.4 Hz, 1H), 6.52 (d, J=8.8 Hz, 2H), 6.47 (dd, J=2.4, 8.4 Hz, 1H), 6.19 (d, J=8.8 Hz, 2H), 4.97 (dd, J=5.2, 13.2 Hz, 1H), 4.48-4.31 (m, 2H), 4.12 (d, J=4.8 Hz, 1H), 3.51-3.48 (m, 2H), 3.31-3.26 (m, 1H), 2.96-2.85 (m, 6H), 2.59-2.51 (m, 2H), 2.50-2.45 (m, 1H), 2.35-2.33 (m, 1H), 2.18-2.16 (m, 3H), 2.01-1.92 (m, 5H), 1.74-1.61 (m, 6H), 1.14-1.12 (m, 2H); MS (ESI) m/z: 729.3 [M+H]⁺.

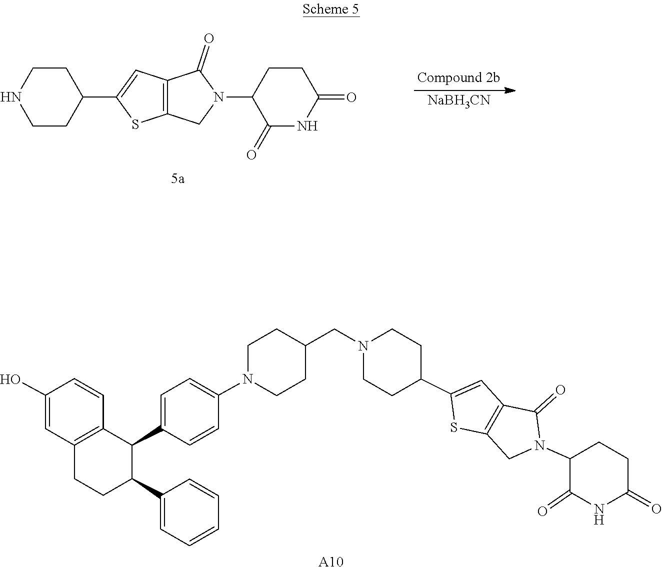 [SMILES: O=C1CCC(N2Cc3sc(C4CCNCC4)cc3C2=O)C(=O)N1 ; "NaBH3CN" ; O=C1CCC(N2Cc3sc(C4CCN(CC5CCN(c6ccc([C@@H]7c8ccc(O)cc8CC[C@@H]7c7ccccc7)cc6)CC5)CC4)cc3C2=O)C(=O)N1 ; "A10"]

### Example 6

### Preparation of 3-(7-(2-(4-(4-(4-((1R,2S)-6-hydroxy-2-phenyl-1,2,3,4-tetrahydronaphthalen-1-yl)-phenoxy)butyl)piperazin-1-yl)-2-oxoethoxy)-1-methyl-1H-indazol-3-yl)piperidine-2,6-dione B1

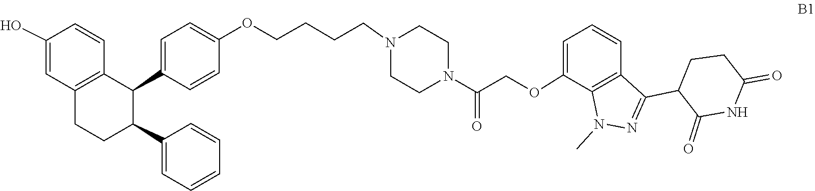 [SMILES: Cn1nc(C2CCC(=O)NC2=O)c2cccc(OCC(=O)N3CCN(CCCCOc4ccc([C@@H]5c6ccc(O)cc6CC[C@@H]5c5ccccc5)cc4)CC3)c21]

Compound B1 was prepared as shown in Scheme 6.

7-(Benzyloxy)-1-methyl-1H-indazol-3-amine 6b. To a solution of 3-(benzyloxy)-2-fluorobenzonitrile 6a (18.16 g, 80 mmol) in DMF (200 mL) were added methylhydrazine (18.42 g, 400 mmol) and Cs₂CO₃ (52.13 g, 160 mmol). After heated in a high-pressure reactor at 120° C. overnight, the reaction was quenched with H₂O (100 mL) and the reaction mixture was extracted with EtOAc (200 mL×2). The combined organic layers were dried over anhydrous Na₂SO₄, filtered, and concentrated to yield a crude product, which was purified by silica gel column chromatography eluting with EtOAc in PE to afford compound 6b (9.55 g) in 47% yield. MS (ESI) m/z: 254.2 [M+H]⁺.

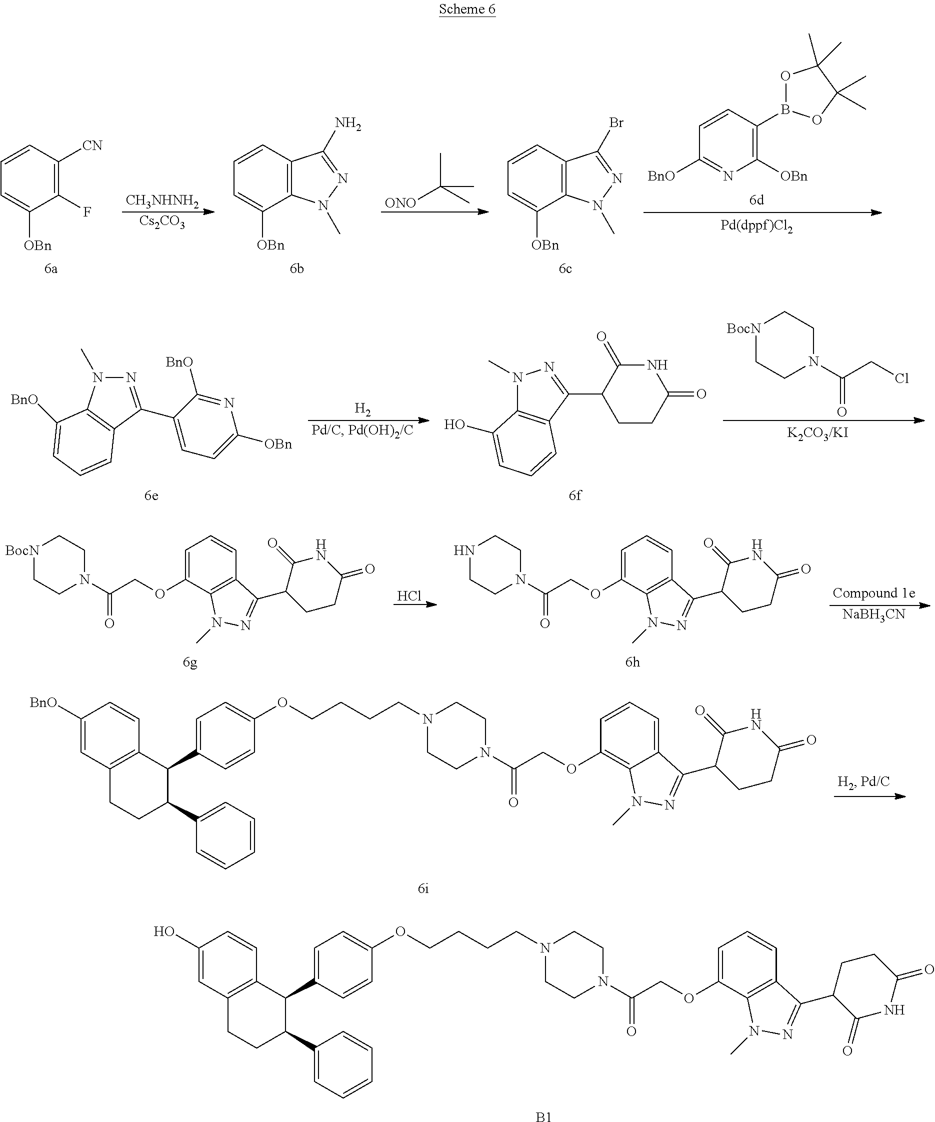

6-(Benzyloxy)-3-bromo-1-methyl-1H-indazole 6c. To a solution of compound 6b (5.0 g, 19.7 mmol) in ACN (30 mL) were added tert-butyl nitrite (3.52 mL, 29.6 mmol) and CuBr (3.674 g, 25.6 mmol). After stirred overnight, the reaction mixture was concentrated to yield a crude product, which was purified by silica gel column chromatography eluting with EtOAc in PE to afford compound 6c (1.8 g) in 21% yield. MS (ESI) m/z: 317.1 [M+H]⁺.

7-(Benzyloxy)-3-(2,6-bis(benzyloxy)pyridin-3-yl)-1-methyl-1H-indazole 6e. To a solution of compound 6c (500 mg, 1.6 mmol) and 2,6-bis(benzyloxy)-3-(4,4,5,5-tetramethyl-1,3,2-dioxaborolan-2-yl)pyridine 6d (1.0 g, 2.4 mmol) in dioxane/water (6:1) (30 mL) was added K₂CO₃ (440 mg, 8.0 mmol) under N₂, followed by addition of Pd(dppf)Cl₂ (115 mg, 0.16 mmol). After heated at 100° C. overnight, the reaction mixture was diluted with water (20 mL) and extracted with EtOAc (30 mL×3). The combined organic layers were dried over anhydrous Na₂SO₄, filtered, and concentrated to yield a crude product, which was purified by silica gel column chromatography eluting with EtOAc in PE to afford compound 6e (800 mg) in 89% yield. MS (ESI) m/z: 528.3 [M+H]⁺.

3-(7-Hydroxy-1-methyl-1H-indazol-3-yl)piperidine-2,6-dione 6f. To a solution of compound 6e (500 mg, 0.95 mmol) in MeOH (10 mL) and THE (10 mL) was added 20% Pd/C (200 mg). After stirred overnight, the reaction mixture was filtered and concentrated to afford compound 6f (200 mg) in 82% yield.

Tert-butyl 4-(2-((3-(2,6-dioxopiperidin-3-yl)-1-methyl-1H-indazol-7-yl)oxy)-acetyl)piperazine-1-carboxylate 6g. To a solution of compound 6f (300 mg, 1.158 mmol) in DMF (10 mL) were added Cs₂CO₃ (451 mg, 1.38 mmol), KI (192 mg, 1.158 mmol), and tert-butyl 4-(2-chloroacetyl)piperazine-1-carboxylate (303 mg, 1.158 mmol). After stirred overnight, the reaction was quenched with water (10 mL) and the reaction mixture was extracted with EtOAc (15 mL×2). The combined organic layers were dried over anhydrous Na₂SO₄, filtered, and concentrated to yield a crude product, which was purified by silica gel column chromatography eluting with EtOAc in PE to afford compound 6g (277 mg) in 49% yield. MS (ESI) m/z: 486.2 [M+H]⁺.

3-(1-Methyl-7-(2-oxo-2-(piperazin-1-yl)ethoxy)-1H-indazol-3-yl)piperidine-2,6-dione 6h. To a solution of compound 6g (80 mg, 0.165 mmol) in DCM (8 mL) was added HCl in EtOAc (2 mL). After stirred for 2 h, the reaction mixture was concentrated to afford compound 6h as a crude product, which was used directly in the next step without further purification. MS (ESI) m/z: 386.5 [M+H]⁺.

3-(7-(2-(4-(4-(4-((1R,2S)-6-(Benzyloxy)-2-phenyl-1,2,3,4-tetrahydronaphthalen-1-yl)phenoxy)butyl)piperazin-1-yl)-2-oxoethoxy)-1-methyl-1H-indazol-3-yl)piperidine-2,6-dione 6i. To a solution of 4-(4-((1R,2S)-6-(benzyloxy)-2-phenyl-1,2,3,4-tetrahydronaphthalen-1-yl)phenoxy)butanal 1e (79 mg, 0.165 mmol) in DMF (10 mL) were added DIEA (19 mg, 0.165 mmol) and compound 6h (crude, 0.165 mmol). After the mixture was stirred for 30 min, NaBH₃CN (31.5 mg, 0.50 mmol) was added. The reaction mixture was stirred for 12 h and then concentrated to yield a crude product, which was purified by silica gel column chromatography eluting with MeOH in DCM to afford compound 6i (60 mg) in 43% yield. MS (ESI) m/z: 846.4 [M+H]⁺.

3-(7-(2-(4-(4-(4-((1R,2S)-6-Hydroxy-2-phenyl-1,2,3,4-tetrahydronaphthalen-1-yl)phenoxy)butyl)piperazin-1-yl)-2-oxoethoxy)-1-methyl-1H-indazol-3-yl)piperidine-2,6-dione B1. To a solution of compound 6i (60 mg, 0.07 mmol) in MeOH (5 mL) and THE (5 mL) was added Pd/C (60 mg). After stirred under H₂ for 12 h, the reaction mixture was filtered and concentrated to yield a crude product, which was purified by reverse phase prep-HPLC to afford compound B1 (7.7 mg) in 15% yield. ¹HNMR (400 MHz, DMSO-d₆) δ 10.87 (s, 1H), 9.16 (s, 1H), 7.24 (d, J=8.4 Hz, 1H), 7.17-7.11 (m, 3H), 6.97 (t, J=8.0 Hz, 1H), 6.84-6.82 (m, 3H), 6.63 (d, J=8.4 Hz, 1H), 6.61 (d, J=2.0 Hz, 1H), 6.53 (d, J=8.4 Hz, 2H), 6.48 (dd, J=2.4, 8.0 Hz, 1H), 6.26 (d, J=8.8 Hz, 2H), 5.01 (s, 2H), 4.32 (dd, J=5.2, 10.0 Hz, 1H), 4.23 (s, 3H), 4.18 (d, J=5.2 Hz, 1H), 3.83 (t, J=5.6 Hz, 2H), 3.51-3.42 (m, 4H), 2.97-2.91 (m, 3H), 2.67-2.53 (m, 3H), 2.42-2.28 (m, 5H), 2.18-2.05 (m, 1H), 2.03-1.97 (m, 1H), 1.72-1.58 (m, 6H); MS (ESI) m/z: 756.5 [M+H]⁺.

### Example 7

### Preparation of 3-(6-(2-(4-(4-(4-((1R,2S)-6-hydroxy-2-phenyl-1,2,3,4-tetrahydronaphthalen-1-yl)phenoxy)butyl)piperazin-1-yl)-2-oxoethoxy)-1-methyl-1H-indazol-3-yl)piperidine-2,6-dione B2

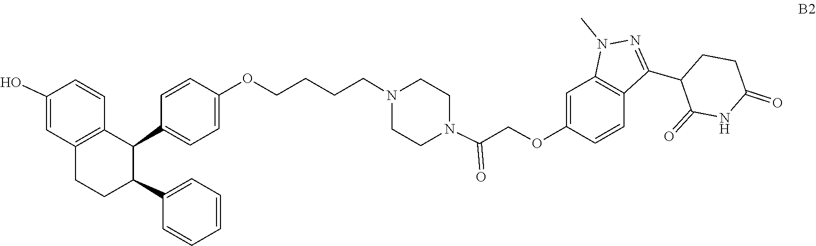 [SMILES: Cn1nc(C2CCC(=O)NC2=O)c2ccc(OCC(=O)N3CCN(CCCCOc4ccc([C@@H]5c6ccc(O)cc6CC[C@@H]5c5ccccc5)cc4)CC3)cc21]

Compound B2 was prepared as shown in Scheme 7.

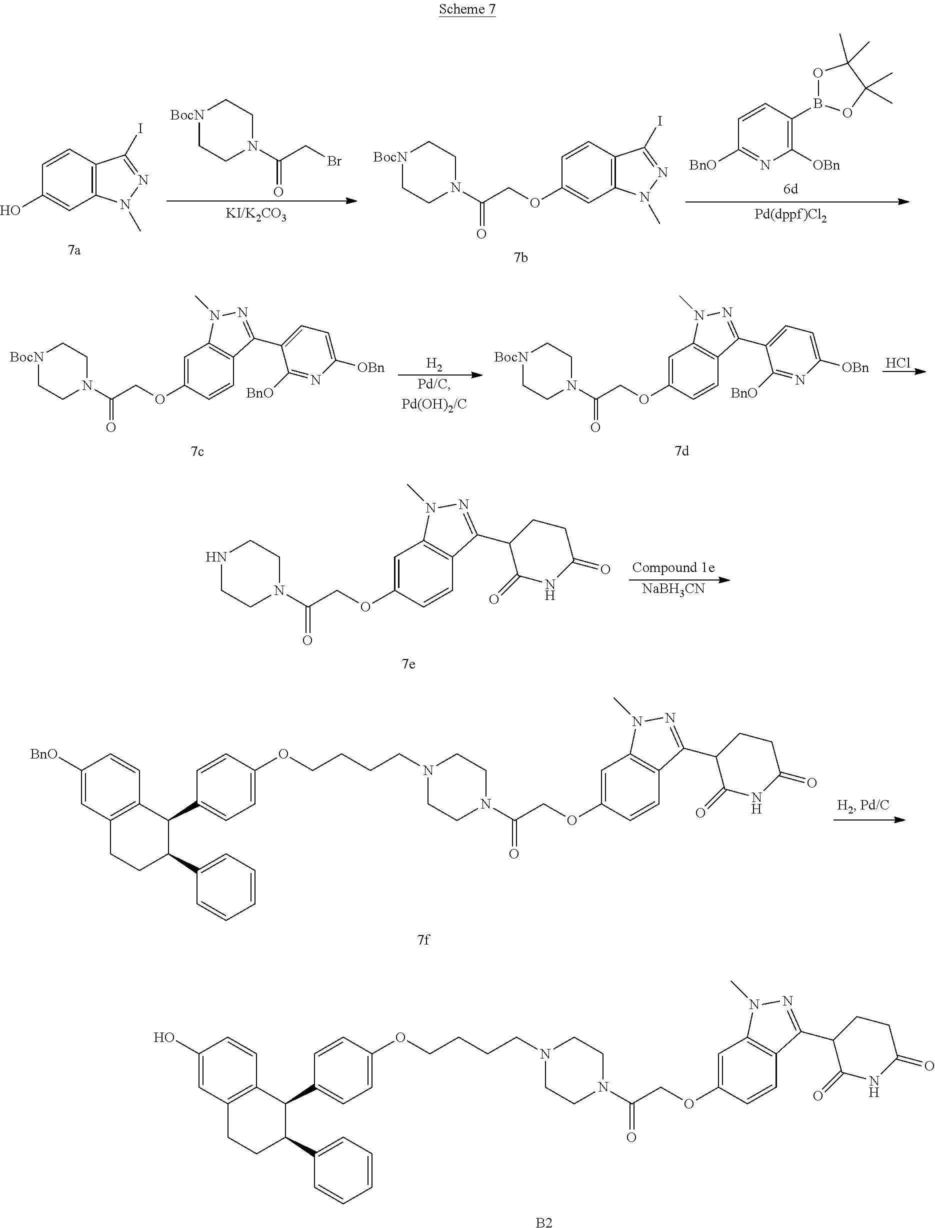

Tert-butyl 4-(2-((3-iodo-1-methyl-1H-indazol-6-yl)oxy)acetyl)piperazine-1-carboxylate 7b. To a solution of 3-iodo-1-methyl-1H-indazol-6-ol 7a (0.48 mmol) in ACN (15 mL) were added tert-butyl 4-(2-chloroacetyl)piperazine-1-carboxylate (97 mg, 0.37 mmol), K₂CO₃ (153 mg, 1.11 mmol), and KI (184 mg, 1.11 mmol). After stirred at 80° C. overnight, the reaction mixture was diluted with H₂O (15 mL) and extracted with EtOAc (40 mL×2). The combined organic layers were dried over anhydrous Na₂SO₄, filtered, and concentrated to yield crude product, which was purified by silica gel column chromatography eluting with EtOAc in PE to afford compound 7b (185 mg) in 77% yield. MS (ESI) m/z: 501.1 [M+H]⁺.

Tert-butyl 4-(2-((3-(2,6-bis(benzyloxy)pyridin-3-yl)-1-methyl-1H-indazol-6-yl)-oxy)acetyl)piperazine-1-carboxylate 7c. To a solution of compound 7b (185 mg, 0.37 mmol) in dioxane (6 mL) and H₂O (1 mL) were added K₂CO₃ (102 mg, 0.74 mmol), 2,6-bis(benzyloxy)-3-(4,4,5,5-tetramethyl-1,3,2-dioxaborolan-2-yl)pyridine 6d (200 mg, 0.48 mmol), and Pd(dppf)Cl₂ (27 mg, 0.04 mmol). After stirred at 85° C. for 4 h, the reaction mixture was diluted with H₂O (15 mL) and extracted with EtOAc (30 mL×2). The combined organic layers were dried over anhydrous Na₂SO₄, filtered, and concentrated to yield a crude product, which was purified by silica gel column chromatography eluting with EtOAc in PE to afford compound 7c (379 mg) in 79% yield. MS (ESI) m/z: 501.1 [M+H]⁺.

Tert-butyl 4-(2-((3-(2,6-dioxopiperidin-3-yl)-1-methyl-1H-indazol-6-yl)oxy)-acetyl)piperazine-1-carboxylate 7d. To a solution of compound 7c (379 mg, 0.57 mmol) in THE (10 mL) and MeOH (10 mL) were added Pd/C (75.8 mg) and Pd(OH)₂/C (75.8 mg). After stirred for 24 h under H₂, the reaction mixture was filtered and concentrated to yield a crude product, which was purified by prep-TLC (EtOAc/PE: 1/3) to afford compound 7d (208 mg) in 75% yield. MS (ESI) m/z: 486.2 [M+H]⁺.

3-(1-Methyl-6-(2-oxo-2-(piperazin-1-yl)ethoxy)-1H-indazol-3-yl)piperidine-2,6-dione 7e. To a solution of compound 7d (92 mg, 0.19 mmol) in DCM (4 mL) was added HCl in EtOAc (2 mL). The reaction mixture was stirred for 1 h and then concentrated to afford compound 7e as a crude product, which was used directly in the next step without further purification. MS (ESI) m/z: 386.2 [M+H]⁺.

3-(6-(2-(4-(4-(4-((1R,2S)-6-(Benzyloxy)-2-phenyl-1,2,3,4-tetrahydronaphthalen-1-yl)phenoxy)butyl)piperazin-1-yl)-2-oxoethoxy)-1-methyl-1H-indazol-3-yl)piperidine-2,6-dione 7f. To a solution of 4-(4-((1R,2S)-6-(benzyloxy)-2-phenyl-1,2,3,4-tetrahydronaphthalen-1-yl)phenoxy)butanal 1e (124 mg, 0.26 mmol) in DMF (10 mL) were added DIEA (33.5 mg, 0.26 mmol) and compound 7e (100 mg, 0.26 mmol). After the mixture was stirred for 30 min, NaBH₃CN (49 mg, 0.78 mmol) was added. The reaction mixture was stirred for 12 h and then concentrated to yield a crude product, which was purified by silica gel column chromatography eluting with MeOH in DCM to afford compound 7f (95 mg) in 43% yield. MS (ESI) m/z: 846.4 [M+H]⁺.

3-(6-(2-(4-(4-(4-((1R,2S)-6-Hydroxy-2-phenyl-1,2,3,4-tetrahydronaphthalen-1-yl)phenoxy)butyl)piperazin-1-yl)-2-oxoethoxy)-1-methyl-1H-indazol-3-yl)piperidine-2,6-dione B2. To a solution of compound 7f (95 mg, 0.11 mmol) in MeOH (5 mL) and THE (5 mL) was added Pd/C (95 mg). After stirred under H₂ for 12 h, the reaction mixture was filtered and concentrated to yield a crude product, which was purified by reverse phase prep-HPLC to afford compound B2 (26.5 mg) in 32% yield. ¹H NMR (400 MHz, DMSO-d₆) δ 10.86 (s, 1H), 9.11 (s, 1H), 7.57 (d, J=8.8 Hz, 1H), 7.17-7.11 (m, 3H), 7.04 (d, J=2.0 Hz, 1H), 6.83 (d, J=6.8 Hz, 2H), 6.77 (dd, J₁=8.4 Hz, J₂=2.0 Hz, 1H), 6.65-6.60 (m, 2H), 6.52 (d, J=8.4 Hz, 2H), 6.48 (dd, J₁=8.4 Hz, J₂=2.4 Hz, 1H), 6.26 (d, J=8.4 Hz, 2H), 4.86 (s, 2H), 4.31 (dd, J₁=5.2 Hz, J₂=9.6 Hz, 1H), 4.17 (d, J=4.4 Hz, 1H), 3.91 (s, 3H), 3.83 (t, J=6.0 Hz, 2H), 3.46 (s, 4H), 3.02-2.91 (m, 2H), 2.67-2.60 (m, 2H), 2.40-2.37 (m, 2H), 2.33-2.30 (m, 6H), 2.18-2.05 (m, 2H), 1.73-1.61 (m, 3H), 1.56-1.49 (m, 2H); MS (ESI) m/z: 756.4 [M+H]⁺.

### Example 8

### Preparation of 3-(7-((4-(((4-(4-((1R,2S)-6-hydroxy-2-phenyl-1,2,3,4-tetrahydronaphthalen-1-yl)-phenoxy)butyl)amino)methyl)benzyl)oxy)-1-methyl-1H-indazol-3-yl)piperidine-2,6-dione B3

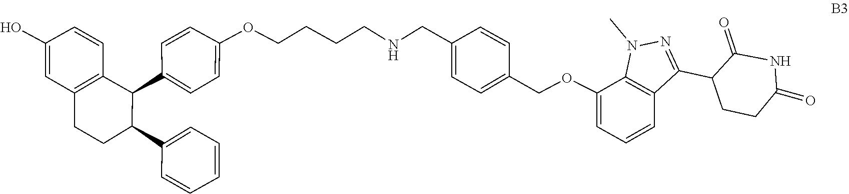 [SMILES: Cn1nc(C2CCC(=O)NC2=O)c2cccc(OCc3ccc(CNCCCCOc4ccc([C@@H]5c6ccc(O)cc6CC[C@@H]5c5ccccc5)cc4)cc3)c21]

Compound B3 is prepared as shown in Scheme 8.

### Example 9

### Preparation of 3-(6-((4-(((4-(4-((1S,2R)-6-hydroxy-2-phenyl-1,2,3,4-tetrahydronaphthalen-1-yl)phenoxy)butyl)amino)methyl)benzyl)oxy)-1-methyl-1H-indazol-3-yl)piperidine-2,6-dione B4

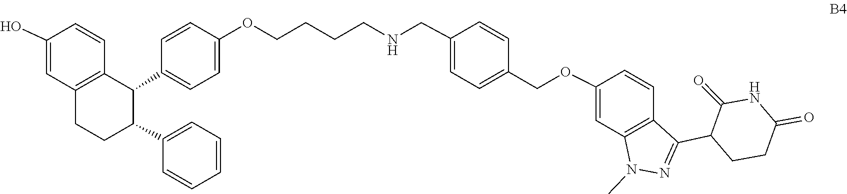 [SMILES: Cn1nc(C2CCC(=O)NC2=O)c2ccc(OCc3ccc(CNCCCCOc4ccc([C@H]5c6ccc(O)cc6CC[C@H]5c5ccccc5)cc4)cc3)cc21]

Compound B4 is prepared as shown in Scheme 9.

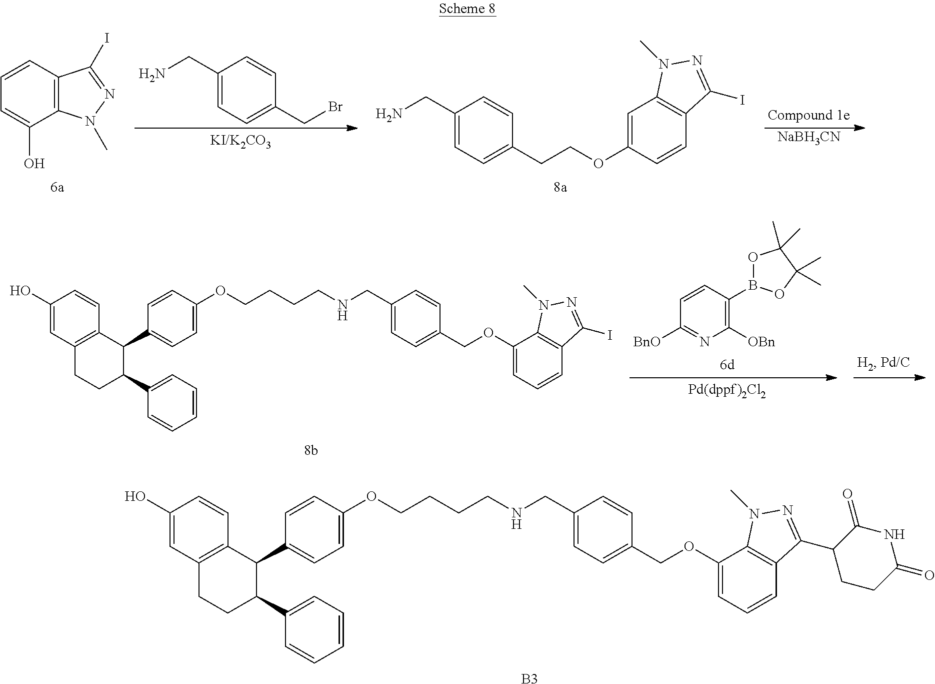

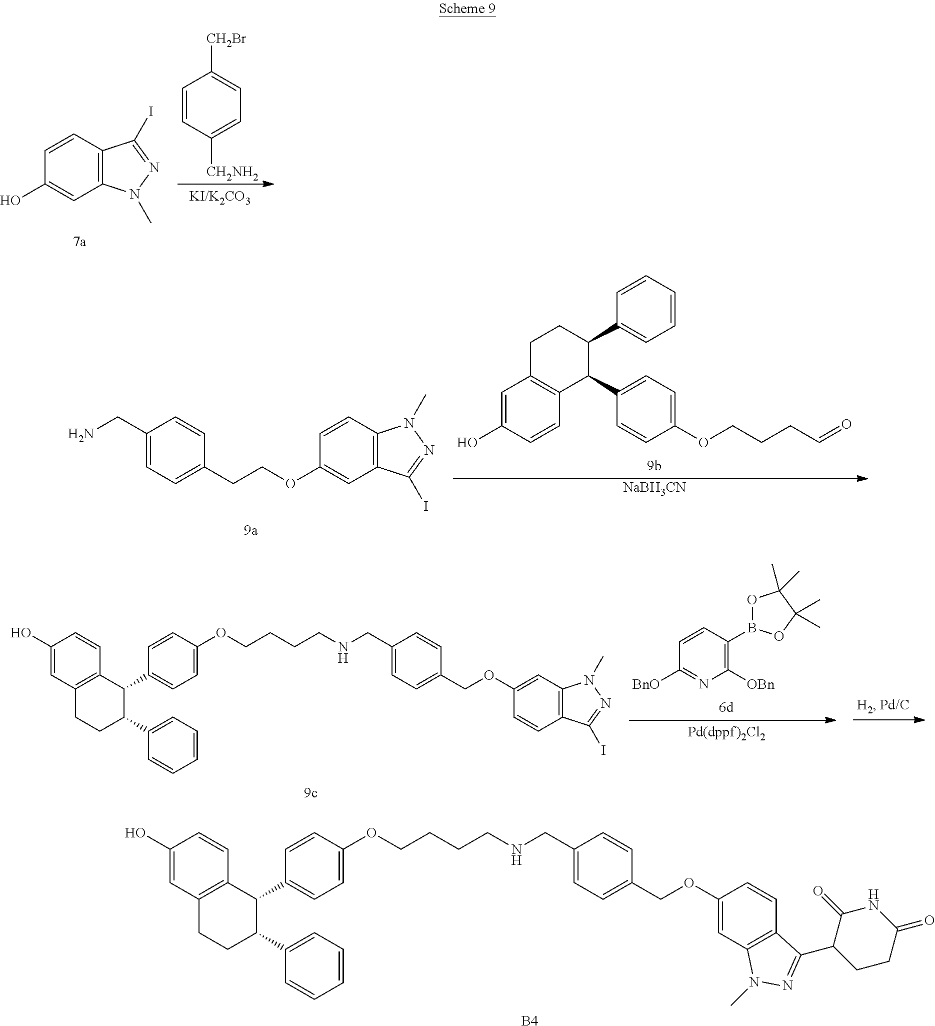

### Example 10

### Preparation of 3-(3-(2-(4-(4-(4-((1R,2S)-6-hydroxy-2-phenyl-1,2,3,4-tetrahydronaphthalen-1-yl)phenoxy)butyl)piperazin-1-yl)-2-oxoethoxy)phenyl)piperidine-2,6-dione B5

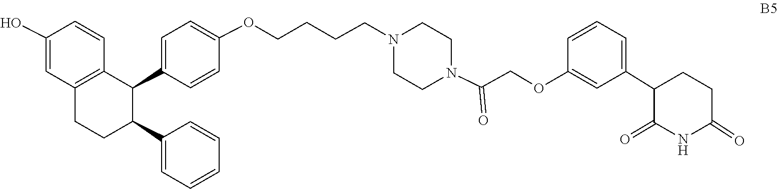 [SMILES: O=C1CCC(c2cccc(OCC(=O)N3CCN(CCCCOc4ccc([C@@H]5c6ccc(O)cc6CC[C@@H]5c5ccccc5)cc4)CC3)c2)C(=O)N1]

Compound B5 was prepared as shown in Scheme 10.

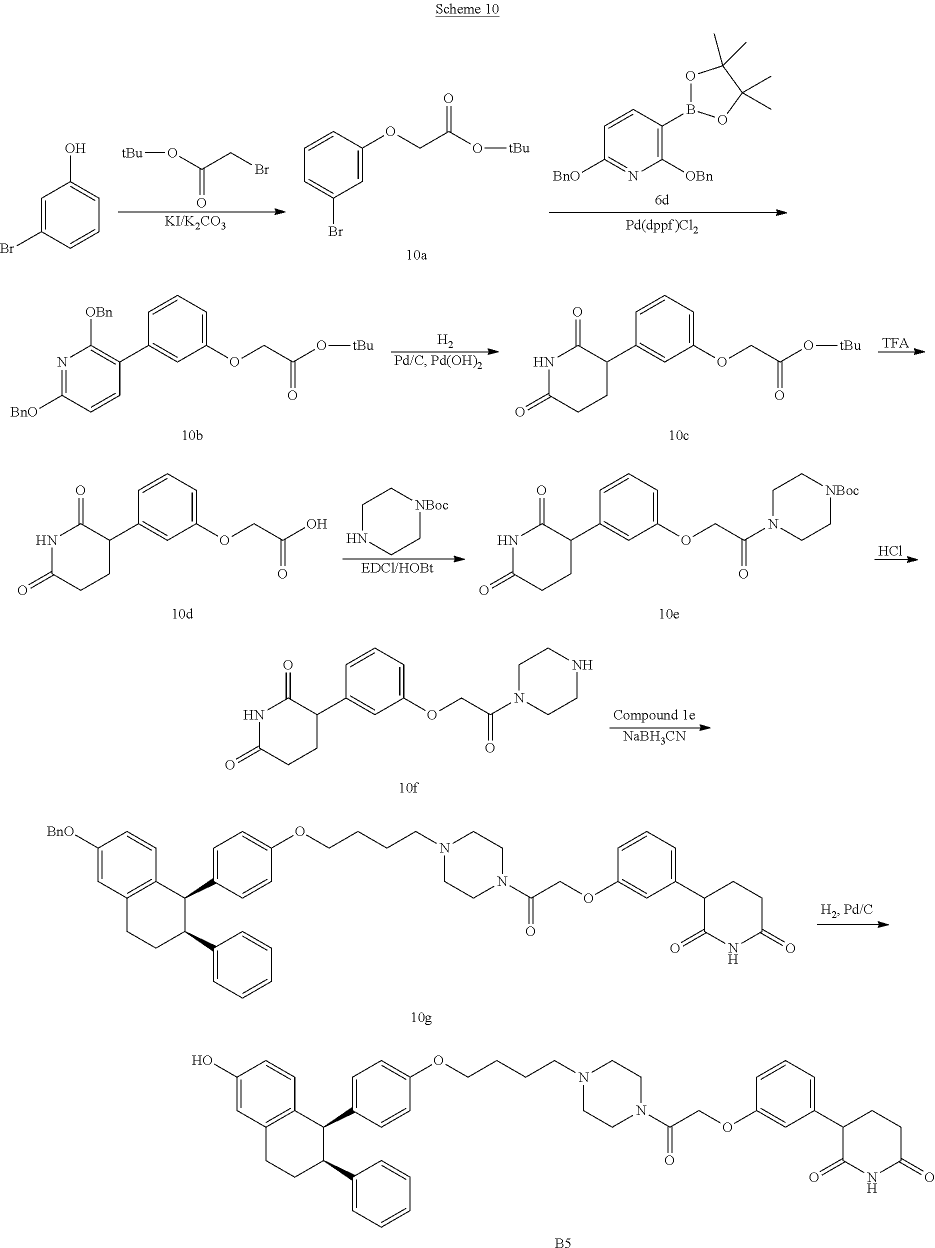

Tert-butyl 2-(3-bromophenoxy)acetate 10a. To a solution of 3-bromophenol (1.0 g, 5.78 mmol) in DMF (18 mL) were added tert-butyl 2-bromoacetate (1.7 g, 8.67 mmol), KI (96.3 mg, 0.58 mmol), and K₂CO₃ (1.6 g, 11.56 mmol). After stirred overnight, the reaction mixture was diluted with H₂O (10 mL) and extracted with EtOAc (40 mL×2). The combined organic layers were dried over anhydrous Na₂SO₄, filtered, and concentrated to afford compound 10a (1.59 g) in 96% yield. ¹H NMR (400 MHz, CDCl₃) δ 7.14 (dd, J=7.6 Hz, 16.8 Hz, 2H), 7.05 (s, 1H), 6.83 (d, J=8.0 Hz, 1H), 4.49 (s, 2H), 1.48 (s, 9H).

Tert-butyl 2-(3-(2,6-bis(benzyloxy)pyridin-3-yl)phenoxy)acetate 10b. To a solution of compound 10a (1.59 g, 5.59 mmol) in dioxane (24 mL) and H₂O (4 mL) were added 2,6-bis(benzyloxy)-3-(4,4,5,5-tetramethyl-1,3,2-dioxaborolan-2-yl) pyridine (3.00 g, 7.20 mmol), K₂CO₃ (1.53 g, 11.08 mmol), and Pd(dppf)Cl₂ (0.41 g, 0.55 mmol). After stirred at 100° C. overnight under N₂, the reaction mixture was diluted with H₂O (20 mL) and extracted with EtOAc (50 mL×3). The combined organic layers were dried over anhydrous Na₂SO₄, filtered, and concentrated to yield a crude product, which was purified by silica gel column chromatography eluting with EtOAc in PE to afford compound 10b (2.03 g) in 74% yield. MS (ESI) m/z: 498.2 [M+H]⁺.

Tert-butyl 2-(3-(2,6-dioxopiperidin-3-yl)phenoxy)acetate 10c. To a solution of compound 10b (250 mg, 0.50 mmol) in THE (5 mL) were added Pd/C (35 mg) and Pd(OH)₂ (35 mg). After stirred overnight under H₂, the reaction mixture was filtered and concentrated to afford compound 10c (152 mg) in 95% yield. MS (ESI) m/z: 337.2 [M+18]⁺.

2-(3-(2,6-Dioxopiperidin-3-yl)phenoxy)acetic acid 10d. To a solution of compound 10c (80 mg, 0.25 mmol) in DCM (4 mL) was added TFA (4 mL). After stirred overnight, the reaction mixture was concentrated to afford compound 10d (75 mg) as a crude product, which was used directly in the next step without further purification. ¹H NMR (400 MHz, DMSO-d₆) δ 10.81 (s, 1H), 7.24 (dd, J=7.2 Hz, 8.8 Hz, 1H), 6.82-6.79 (m, 3H), 4.65 (s, 2H), 3.83 (dd, J=5.2 Hz, 11.6 Hz, 1H), 2.66-2.60 (m, 1H), 2.51-2.45 (m, 1H), 2.21-2.17 (m, 1H), 2.07-2.00 (m, 1H); MS (ESI) m/z: 264.1 [M+H]⁺.

Tert-butyl 4-(2-(3-(2,6-dioxopiperidin-3-yl)phenoxy)acetyl)piperazine-1-carboxylate 10e. To a solution of compound 10d (100 mg, 0.38 mmol) and tert-butyl piperazine-1-carboxylate (70 mg, 0.38 mmol) in DMF (4 mL) were added HOBt (84 mg, 0.57 mmol), EDCI·HCl (109 mg, 0.57 mmol) and DIEA (148 mg, 1.14 mmol). After stirred for 12 h, the reaction mixture was diluted with H₂O (10 mL) and extracted with EtOAc (15 mL×2). The combined organic layers were dried over anhydrous Na₂SO₄, filtered, and concentrated to yield a crude product, which was purified by silica gel column chromatography eluting with MeOH in DCM to afford 10e (98 mg) in 60% yield. MS (ESI) m/z: 432.2[M+H]⁺.

3-(3-(2-Oxo-2-(piperazin-1-yl)ethoxy)phenyl)piperidine-2,6-dione 10f. To a solution of compound 10e (150 mg, 0.28 mmol) in DCM (4 mL) was added HCl in EtOAc (1 mL) with dropwise. After stirred for 1 h, the reaction mixture was concentrated to afford compound 10f as a crude product, which was used directly in the next step without further purification. MS (ESI) m/z: 332.2 [M+H]⁺.

3-(3-(2-(4-(4-(4-((1R,2S)-6-(Benzyloxy)-2-phenyl-1,2,3,4-tetrahydronaphthalen-1-yl)phenoxy)butyl)piperazin-1-yl)-2-oxoethoxy)phenyl)piperidine-2,6-dione 10g. To a solution of 4-(4-((1R,2S)-6-(benzyloxy)-2-phenyl-1,2,3,4-tetrahydronaphthalen-1-yl)phenoxy)butanal 1e (120 mg, crude) in DMF (8 mL) was added compound 10f (92 mg, 0.28 mmol) at 0° C. After the mixture was stirred at 0° C. for 1 h, NaBH₃CN (35 mg, 0.56 mmol) was added. The reaction mixture was stirred at room temperature for 8 h and then concentrated to yield a crude product, which was purified by silica gel column chromatography eluting with MeOH in DCM to afford 10g (144 mg) in 65% yield. MS (ESI) m/z: 793.1 [M+1]⁺.

3-(3-(2-(4-(4-(4-((1R,2S)-6-Hydroxy-2-phenyl-1,2,3,4-tetrahydronaphthalen-1-yl)phenoxy)butyl)piperazin-1-yl)-2-oxoethoxy)phenyl)piperidine-2,6-dione B5. To a solution of compound 10g (144 mg, 0.182 mmol) in THE (10 mL) was added Pd/C (40 mg). After stirred under H₂ for 8 h, the reaction mixture was filtered and concentrated to yield a crude product, which was purified by silica gel column chromatography eluting with MeOH in DCM to afford compound B5 (85 mg) in 66% yield. ¹H NMR (400 MHz, DMSO-d₆) δ 10.82 (s, 1H), 9.18 (s, 1H), 7.22 (t, J=8.8 Hz, 1H), 7.17-7.11 (m, 3H), 6.83-6.79 (m, 5H), 6.65-6.60 (m, 2H), 6.53-6.47 (m, 3H), 6.26 (d, J=8.4 Hz, 2H), 4.77 (s, 2H), 4.17 (d, J=5.2 Hz, 1H), 3.84-3.79 (m, 3H), 3.42 (s, 4H), 3.30 (dd, J=3.2, 11.6 Hz, 1H), 3.00-2.91 (m, 2H), 2.67-2.60 (m, 1H), 2.46-2.44 (m, 1H), 2.36 (s, 2H), 2.32-2.29 (m, 4H), 2.20-2.16 (m, 1H), 2.08-2.00 (m, 2H), 1.72-1.62 (m, 3H), 1.53-1.50 (m, 2H); MS (ESI) m/z: 702.5 [M+H]⁺.

### Example 11

### Preparation of 3-(4-(2-(4-(4-(4-((1R,2S)-6-hydroxy-2-phenyl-1,2,3,4-tetrahydronaphthalen-1-yl)phenoxy)butyl)piperazin-1-yl)-2-oxoethoxy)phenyl)piperidine-2,6-dione B6

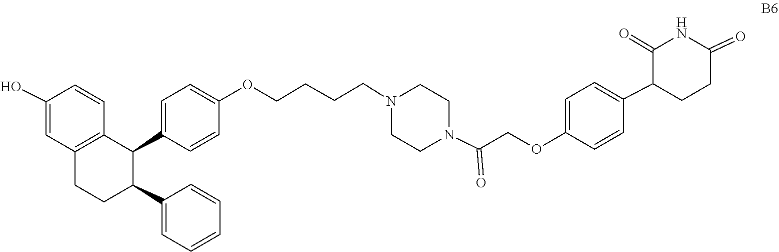 [SMILES: O=C1CCC(c2ccc(OCC(=O)N3CCN(CCCCOc4ccc([C@@H]5c6ccc(O)cc6CC[C@@H]5c5ccccc5)cc4)CC3)cc2)C(=O)N1]

Compound B6 was prepared as shown in Scheme 11.

Tert-butyl 2-(4-bromophenoxy)acetate 11a. To a solution of 4-bromophenol (1.0 g, 5.8 mmol) and tert-butyl 2-bromoacetate (1.7 g, 8.7 mmol) in DMF (10 mL) was added K₂CO₃ (2.4 g, 17.3 mmol). After stirred overnight, the reaction mixture was diluted with H₂O (20 mL) and extracted with EtOAc (30 mL×3). The combined organic layers were dried over anhydrous Na₂SO₄, filtered, and concentrated to afford compound 11a (1.7 g) as a crude product, which was used directly in the next step without further purification.

Tert-butyl 2-(4-(2,6-bis(benzyloxy)pyridin-3-yl)phenoxy)acetate 11b. To a solution of compound 11a (1.7 g, 5.8 mmol) and 2,6-bis(benzyloxy)-3-(4,4,5,5-tetramethyl-1,3,2-dioxaborolan-2-yl)pyridine 6d (2.9 g, 6.9 mmol) in dioxane (16 mL) and water (4 mL) was added K₂CO₃ (1.6 g, 11.6 mmol), followed by addition of Pd(dppf)Cl₂ (219 mg, 0.3 mmol). After heated at 100° C. overnight, the reaction mixture was diluted with water (20 mL) and extracted with EtOAc (30 mL×3). The combined organic layers were dried over anhydrous Na₂SO₄, filtered, and concentrated to yield a crude product, which was purified by silica gel column chromatography eluting with EtOAc in PE to afford compound 11b (1.7 g) in 59% yield. MS (ESI) m/z: 498.2 [M+H]⁺.

Tert-butyl 2-(4-(2,6-dioxopiperidin-3-yl)phenoxy)acetate 11c. To a solution of compound 11b (1.7 g, 3.4 mmol) in MeOH/DCM (5/1) (25 mL) was added 10% Pd/C (200 mg). After stirred overnight under H₂, the reaction mixture was filtered and concentrated to afford compound 11c (925 mg) in 85% yield. MS (ESI) m/z: 342.1 [M+Na]⁺.

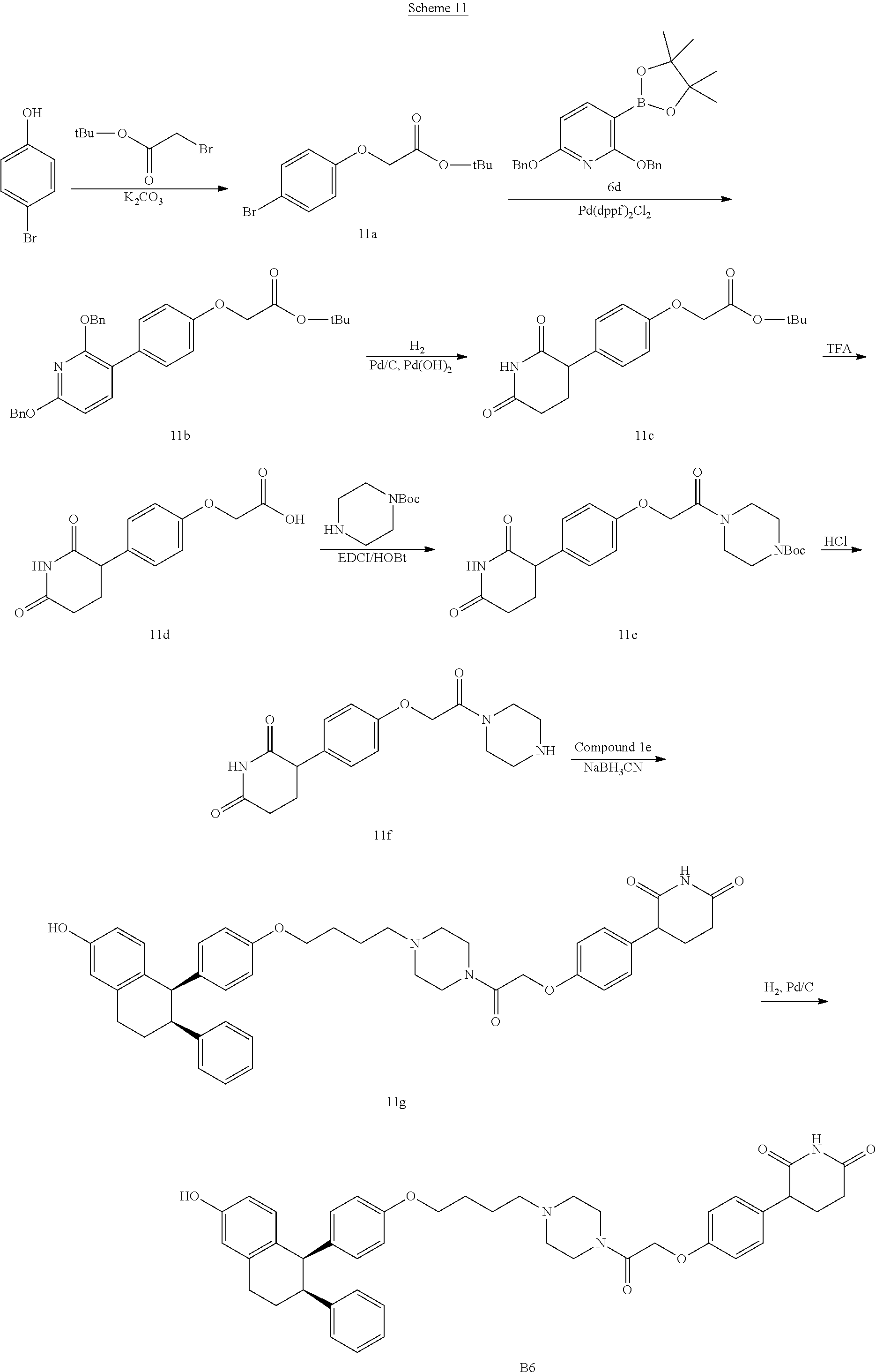

2-(4-(2,6-Dioxopiperidin-3-yl)phenoxy)acetic acid 11d. To a solution of compound 11c (200 mg, 0.63 mmol) in DCM (8 mL) was added TFA (2 mL). After stirred overnight, the reaction mixture was concentrated, diluted with water (5 mL), adjusted to pH 9 with Na₂CO₃, and extracted with DCM (5 mL×3). The aqueous phase was adjusted to pH 4 with 1N HCl. The resulting precipitates were collected by filtration, washed with water, and dried to afford compound 11d (103 mg) in 62% yield. ¹H NMR (400 MHz, DMSO-d₆) δ 12.97 (s, 1H), 10.78 (s, 1H), 7.13 (d, J=8.8 Hz, 2H), 6.86 (d, J=8.8 Hz, 2H), 4.65 (s, 2H), 3.80-3.76 (m, 1H), 2.68-2.61 (m, 1H), 2.51-2.45 (m, 1H), 2.18-2.14 (m, 1H), 2.03-1.98 (m, 1H); MS (ESI) m/z: 264.1 [M+H]⁺.

Tert-butyl 4-(2-(4-(2,6-dioxopiperidin-3-yl)phenoxy)acetyl)piperazine-1-carboxylate 11e. To a solution of compound 11d (70 mg, 0.38 mmol) in DMF (4 mL) were added HOBt (84 mg, 0.57 mmol), EDCI·HCl (109 mg, 0.57 mmol) and DIEA (148 mg, 1.14 mmol). After stirred for 12 h, the reaction mixture was diluted with H₂O (10 mL) and extracted with EtOAc (15 mL×2). The combined organic layers were dried over anhydrous Na₂SO₄, filtered, and concentrated to yield a crude product, which was purified by silica gel column chromatography eluting with MeOH in DCM to afford compound 11e (98 mg) in 60% yield. MS (ESI) m/z: 432.2 [M+H]⁺.

3-(4-(2-Oxo-2-(piperazin-1-yl)ethoxy)phenyl)piperidine-2,6-dione 11f. To a solution of compound 11e (100 mg, 0.23 mmol) in DCM (5 mL) was added HCl in EtOAc (2 mL) dropwise. After stirred for 2 h, the reaction mixture was concentrated to afford compound 11f as a crude product, which was used directly in the next step without further purification. MS (ESI) m/z: 332.1 [M+H]⁺.

3-(4-(2-(4-(4-(4-((1R,2S)-6-(Benzyloxy)-2-phenyl-1,2,3,4-tetrahydronaphthalen-1-yl)phenoxy)butyl)piperazin-1-yl)-2-oxoethoxy)phenyl)piperidine-2,6-dione 11g. To a solution of 4-(4-((1R,2S)-6-(benzyloxy)-2-phenyl-1,2,3,4-tetrahydronaphthalen-1-yl)phenoxy)butanal 1e (100 mg, 0.21 mmol) in DCM (8 mL) and MeOH (2 mL) were added 3-(4-(2-oxo-2-(piperazin-1-yl)ethoxy)phenyl)piperidine-2,6-dione 11f (110 mg, 0.23 mmol) and NaBH₃CN (39 mg, 0.63 mmol). After stirred for 8 h, the reaction mixture was concentrated to yield a crude product, which was purified by silica gel column chromatography eluting with MeOH in DCM to afford 11g (85 mg) 51% yield. MS (ESI) m/z: 792.1 [M+H]⁺.

3-(4-(2-(4-(4-(4-((1R,2S)-6-Hydroxy-2-phenyl-1,2,3,4-tetrahydronaphthalen-1-yl)phenoxy)butyl)piperazin-1-yl)-2-oxoethoxy)phenyl)piperidine-2,6-dione B6. To a solution of compound 11g (85 mg, 0.107 mmol) in THE (10 mL) was added Pd/C (85 mg). After stirred at under H₂ for 8 h, the reaction mixture was filtered and concentrated to yield a crude product, which was purified by silica gel column chromatography eluting with MeOH in DCM to afford compound B6 (42 mg) in 59% yield. ¹H NMR (400 MHz, DMSO-d₆) δ 10.79 (s, 1H), 9.11 (s, 1H), 7.17-7.10 (m, 5H), 6.87-6.82 (m, 4H), 6.65-6.60 (m, 2H), 6.54-6.49 (m, 3H), 6.26 (d, J=8.8 Hz, 2H), 4.78 (s, 2H), 4.18 (d, J=4.8 Hz, 1H), 3.84-3.76 (m, 3H), 3.42 (s, 4H), 3.28-3.25 (m, 1H), 2.98-2.91 (m, 2H), 2.65-2.61 (m, 1H), 2.49-2.28 (m, 7H), 2.17-2.11 (m, 2H), 2.02-1.99 (m, 1H), 1.68-1.63 (m, 3H), 1.55-1.51 (m, 2H); MS (ESI) m/z: 702.5 [M+H]⁺.

### Example 12

### Preparation of 3-(3-((4-(((4-(4-((1S,2R)-6-hydroxy-2-phenyl-1,2,3,4-tetrahydronaphthalen-1-yl)phenoxy)butyl)amino)methyl)benzyl)oxy)phenyl)piperidine-2,6-dione B7

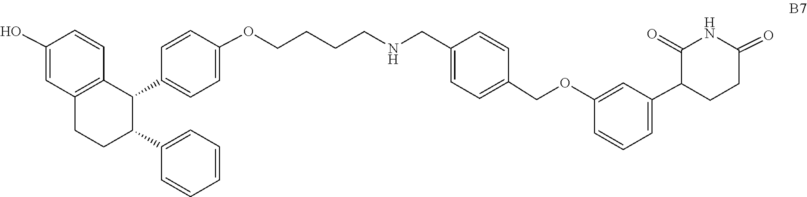 [SMILES: O=C1CCC(c2cccc(OCc3ccc(CNCCCCOc4ccc([C@H]5c6ccc(O)cc6CC[C@H]5c5ccccc5)cc4)cc3)c2)C(=O)N1]

Compound B7 is prepared as shown in Scheme 12.

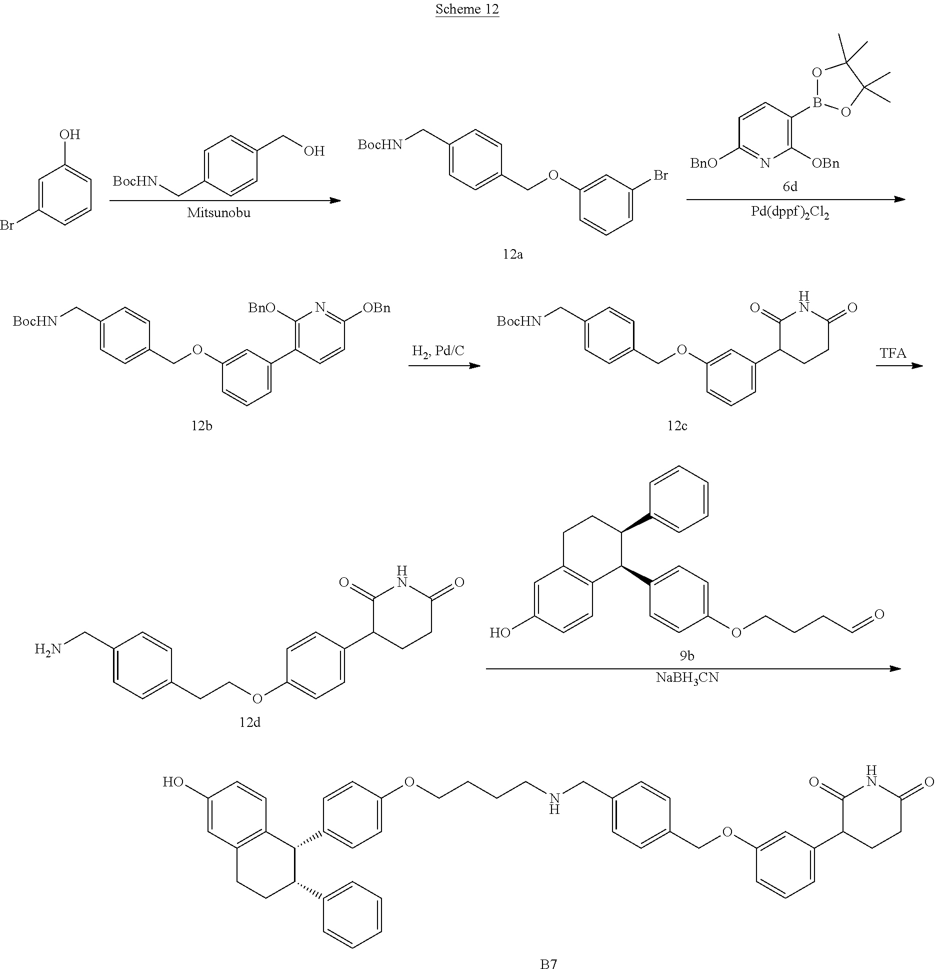

### Example 13

### Preparation of 3-(4-((4-(((4-(4-((1S,2R)-6-hydroxy-2-phenyl-1,2,3,4-tetrahydronaphthalen-1-yl)phenoxy)butyl)amino)methyl)benzyl)oxy)phenyl)piperidine-2,6-dione B8

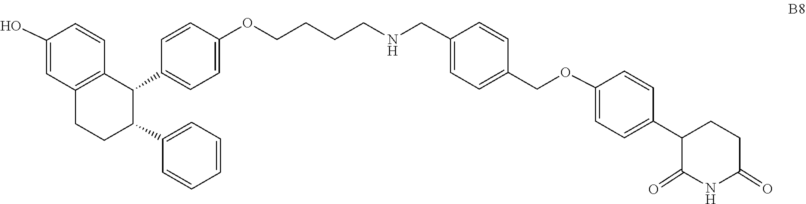 [SMILES: O=C1CCC(c2ccc(OCc3ccc(CNCCCCOc4ccc([C@H]5c6ccc(O)cc6CC[C@H]5c5ccccc5)cc4)cc3)cc2)C(=O)N1]

Compound B8 is prepared as shown in Scheme 13.

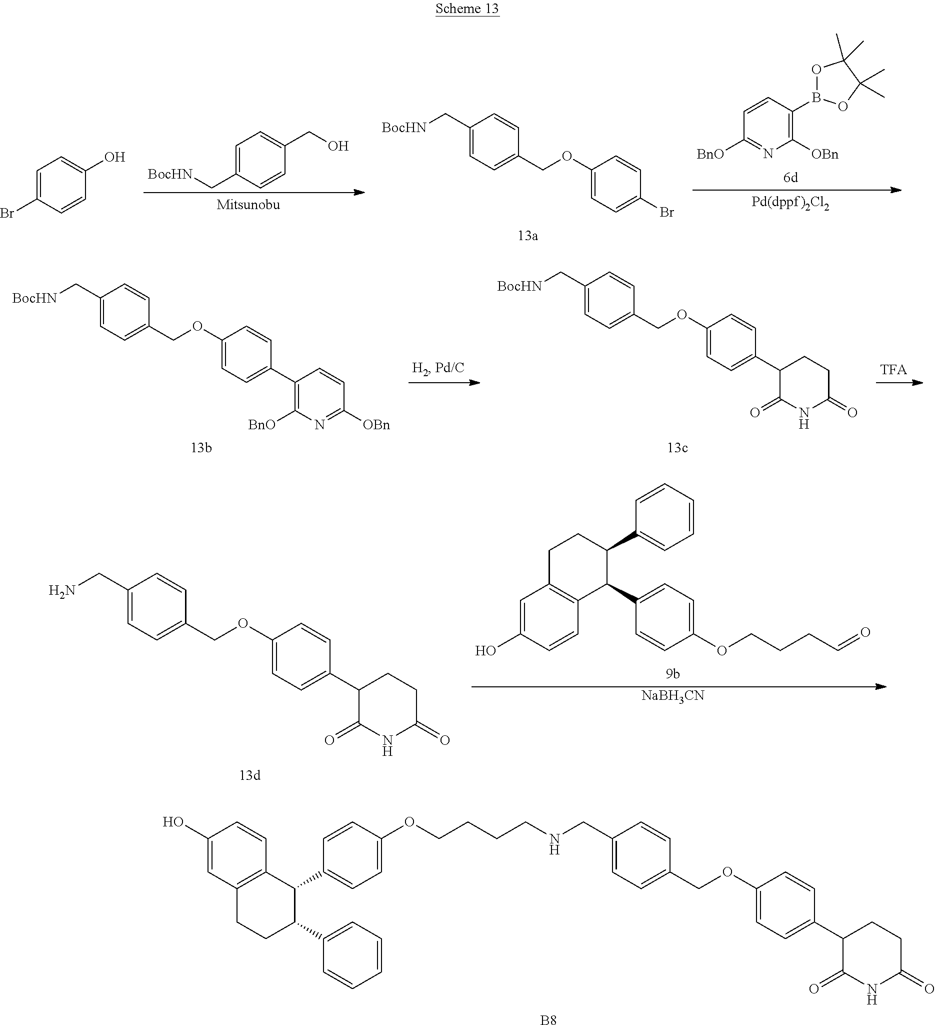

### Example 14

### Preparation of 1-(3-(2-(4-(4-(4-((1R,2S)-6-hydroxy-2-phenyl-1,2,3,4-tetrahydronaphthalen-1-yl)phenoxy)butyl)piperazin-1-yl)-2-oxoethoxy)phenyl)dihydropyrimidine-2,4(1H,3H)-dione B9

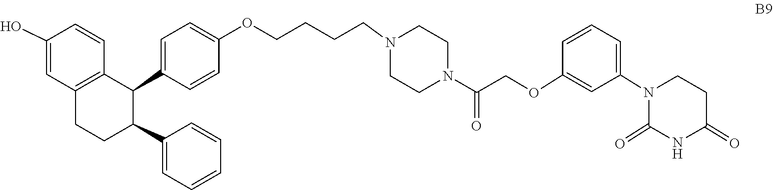 [SMILES: O=C1CCN(c2cccc(OCC(=O)N3CCN(CCCCOc4ccc([C@@H]5c6ccc(O)cc6CC[C@@H]5c5ccccc5)cc4)CC3)c2)C(=O)N1]

Compound B9 was prepared as shown in Scheme 14.

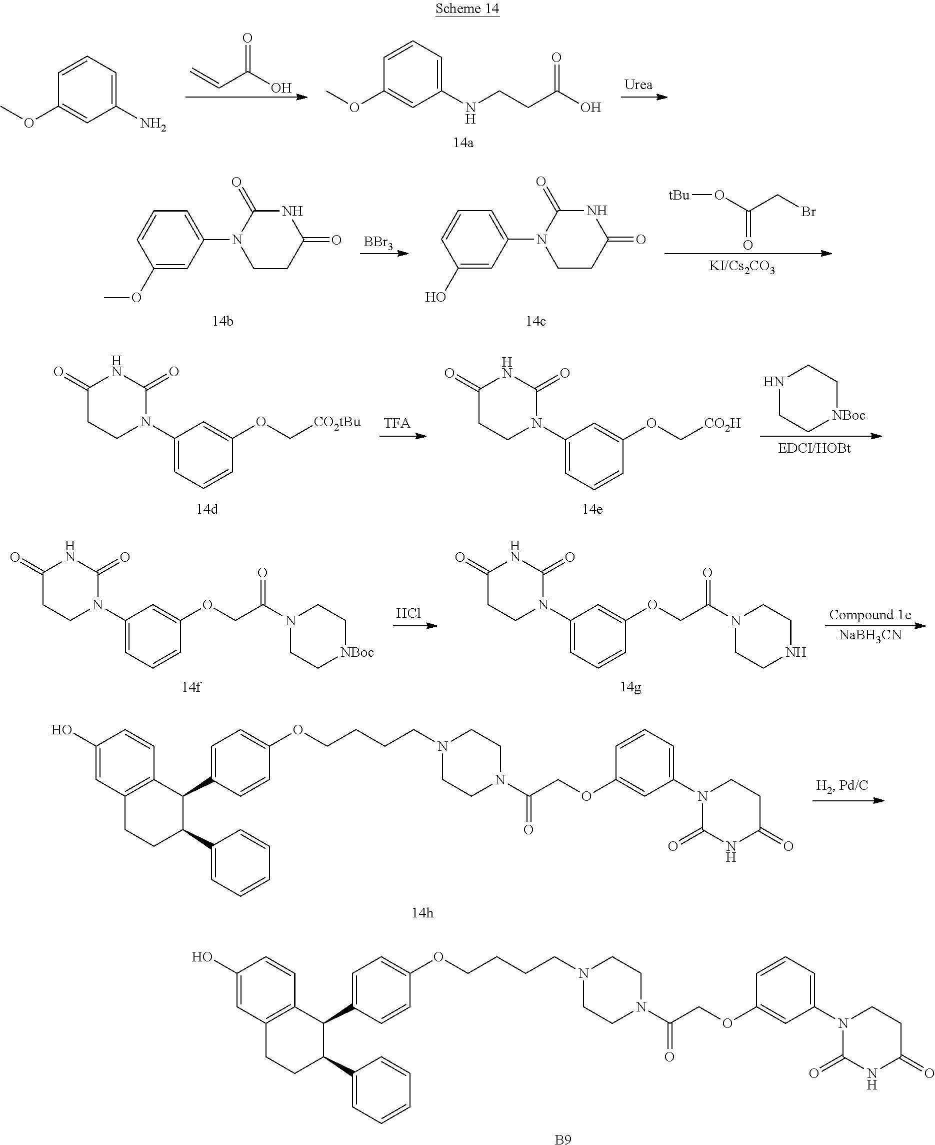

3-((3-Methoxyphenyl)amino)propanoic acid 14a. To a solution of 3-methoxyaniline (25 g, 0.2 mol) in toluene (400 mL) was added acrylic acid (22 g, 0.3 mol). The reaction mixture was stirred at 100° C. overnight and then concentrated to afford compound 14a (40 g) as a crude product, which was used directly in the next step without further purification. MS (ESI) m/z: 196.1 [M+H]⁺.

1-(3-Methoxyphenyl)dihydropyrimidine-2,4(1H,3H)-dione 14b. To a solution of compound 14a (40 g, crude) in AcOH (300 mL) was added urea (40 g, 0.66 mol). After stirred at 120° C. overnight, the reaction mixture was concentrated to yield a crude product, which was triturated with EtOAc to afford compound 14b (22.3 g) in 50% yield. MS (ESI) m/z: 221.1 [M+H]⁺.

1-(3-Hydroxyphenyl)dihydropyrimidine-2,4(1H,3H)-dione 14c. To a solution of compound 14b (5 g, 20 mmol) in DCM (50 mL) at −70° C. was added BBr₃ (157 mL, 220 mmol) dropwise over 30 min. After stirred for 2 h, the reaction was quenched with water (30 mL) and the reaction mixture was extracted with EtOAc (50 mL×2). The combined organic layers were dried over anhydrous Na₂SO₄, filtered, and concentrated to afford compound 14c (4 g) in 97% yield. MS (ESI) m/z: 207.2 [M+H]⁺.

Tert-butyl 2-(3-(2,4-dioxotetrahydropyrimidin-1(2H)-yl)phenoxy)acetate 14d. To a solution of compound 14c (500 mg, 2.42 mmol) in DMF (5 mL) were added Cs₂CO₃ (1.18 g, 3.63 mmol), KI (200 mg, 1.2 mmol), and tert-butyl 2-bromoacetate (378 mg, 1.9 mmol). After stirred overnight, the reaction was quenched with water (10 mL) and the reaction mixture was extracted with EtOAc (30 mL×2). The combined organic layers were dried over anhydrous Na₂SO₄, filtered, and concentrated to yield a crude product, which was purified by silica gel column chromatography eluting with EtOAc in PE to afford compound 14d (300 mg) in 38% yield. MS (ESI) m/z: 265.1 [M-Boc]⁺.

2-(3-(2,4-Dioxotetrahydropyrimidin-1(2H)-yl)phenoxy)acetic acid 14e. To a solution of compound 14d (150 mg, 0.47 mmol) in DCM (3 mL) was added TFA (1 mL). After stirred overnight, the reaction mixture was concentrated to yield a crude product, which was purified by reverse phase prep-HPLC to afford compound 14e (98 mg) in 79% yield. ¹H NMR (400 MHz, DMSO-d₆) δ 10.35 (s, 1H), 7.28 (t, J=8.4 Hz, 1H), 6.94-6.92 (m, 2H), 6.80-6.77 (m, 1H), 4.67 (s, 2H), 3.77 (t, J=6.8 Hz, 2H), 2.70 (t, J=6.8 Hz, 2H); MS (ESI) m/z: 265.1 [M+H]⁺.

Tert-butyl 4-(2-(3-(2,4-dioxotetrahydropyrimidin-1(2H)-yl)phenoxy)acetyl)-piperazine-1-carboxylate 14f. To a solution of compound 14e (150 mg, 0.57 mmol) and tert-butyl piperazine-1-carboxylate (106 mg, 0.57 mmol) in DMF were added HOBt (115 mg, 0.86 mmol), EDCI·HCl (165 mg, 0.86 mmol), and DIEA (220 mg, 1.17 mmol). After stirred overnight, the reaction mixture was diluted with H₂O (10 mL) and extracted with EtOAc (15 mL×2). The combined organic layers were dried over anhydrous Na₂SO₄, filtered, and concentrated to yield a crude product, which was purified by silica gel column chromatography eluting with MeOH in DCM to afford compound 14f (172 mg) in 70% yield. MS (ESI) m/z: 433.2 [M+H]⁺.

3-(4-(2-Oxo-2-(piperazin-1-yl)ethoxy)phenyl)piperidine-2,6-dione 14g. To a solution of compound 14f (100 mg, 0.23 mmol) in DCM (8 mL) were added HCl in EtOAc (2 mL) dropwise. After stirred for 2 h, the reaction mixture was concentrated to afford compound 14g (80 mg) as a crude product, which was used directly in the next step without further purification. MS (ESI) m/z: 333.1 [M+H]⁺.

1-(3-(2-(4-(4-(4-((1R,2S)-6-(Benzyloxy)-2-phenyl-1,2,3,4-tetrahydronaphthalen-1-yl)phenoxy)butyl)piperazin-1-yl)-2-oxoethoxy)phenyl)dihydropyrimidine-2,4(1H,3H)-dione 14h. To a solution of 4-(4-((1R,2S)-6-(benzyloxy)-2-phenyl-1,2,3,4-tetrahydronaphthalen-1-yl)phenoxy)butanal 1e (100 mg, crude) in DCM (5 mL) and MeOH (0.5 mL) were added compound 14g (93 mg, 0.28 mmol) and NaBH₃CN (36 mg, 0.56 mmol). After stirred for 12 h, the reaction mixture was concentrated to yield a crude product, which was purified by silica gel column chromatography eluting with MeOH in DCM to afford compound 14h (167 mg) in 75% yield. MS (ESI) m/z: 793.3 [M+H]⁺.

1-(3-(2-(4-(4-(4-((1R,2S)-6-Hydroxy-2-phenyl-1,2,3,4-tetrahydronaphthalen-1-yl)phenoxy)butyl)piperazin-1-yl)-2-oxoethoxy)phenyl)dihydropyrimidine-2,4(1H,3H)-dione B9. To a solution of compound 14h (167 mg, 0.20 mmol) in MeOH (3 mL) and THE (3 mL) was added Pd/C (33 mg). After stirred under H₂ for 12 h, the reaction mixture was filtered and concentrated to yield a crude product, which was purified by reverse phase prep-HPLC to afford compound B9 (56.4 mg) in 38% yield. ¹H NMR (400 MHz, DMSO-d₆) δ 10.37 (s, 1H), 10.24 (s, 1H), 9.13 (s, 1H), 7.28 (t, J=8.0 Hz, 1H), 7.15-7.11 (m, 3H), 6.94-6.91 (m, 2H), 6.84-6.80 (m, 3H), 6.64-6.61 (m, 2H), 6.54 (d, J=8.8 Hz, 2H), 6.48 (dd, J=2.4, 8.0 Hz, 1H), 6.27 (d, J=8.4 Hz, 2H), 4.88 (d, J=14.0 Hz, 2H), 4.40-4.37 (m, 1H), 4.19 (d, J=4.8 Hz, 2H), 3.85 (t, J=6.0 Hz, 2H), 3.77 (t, J=6.4 Hz, 2H), 3.49 (d, J=12.4 Hz, 2H), 3.32-3.28 (m, 2H), 3.13-3.01 (m, 4H), 2.96-2.85 (m, 3H), 2.70 (t, J=6.8 Hz, 2H), 2.03-1.97 (m, 1H), 1.79-1.68 (m, 5H); MS (ESI) m/z: 703.3 [M+H]⁺.

### Example 15

### Preparation of 1-(4-(2-(4-(4-(4-((1R,2S)-6-hydroxy-2-phenyl-1,2,3,4-tetrahydronaphthalen-1-yl)phenoxy)butyl)piperazin-1-yl)-2-oxoethoxy)phenyl)dihydropyrimidine-2,4(1H,3H)-dione B10

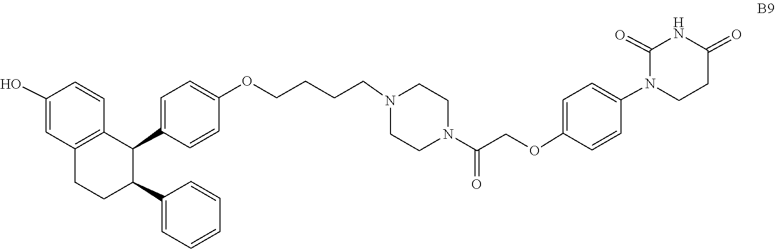 [SMILES: O=C1CCN(c2ccc(OCC(=O)N3CCN(CCCCOc4ccc([C@@H]5c6ccc(O)cc6CC[C@@H]5c5ccccc5)cc4)CC3)cc2)C(=O)N1]

Compound B10 was prepared as shown in Scheme 15.

3-((4-Methoxyphenyl)amino)propanoic acid 15a. To a solution of 4-methoxyaniline (40 g, 0.325 mol) in toluene (400 mL) was added acrylic acid (35.1 g, 0.488 mol). After stirred at 100° C. overnight, the reaction mixture was concentrated to afford compound 15a (63.3 g) as a crude product, which was used directly in the next step without further purification. MS (ESI) m/z: 196.1 [M+H]⁺.

1-(4-Methoxyphenyl)dihydropyrimidine-2,4(1H,3H)-dione 15b. To a solution of compound 15a (63.3 g, crude) in AcOH (500 mL) was added urea (64.4 g, 1.073 mol). After stirred at 120° C. overnight, the reaction mixture was concentrated to yield a crude product, which was triturated with EtOAc to afford compound 15b (50 g) in 70% yield. MS (ESI) m/z: 221.2 [M+H]⁺.

1-(4-Hydroxyphenyl)dihydropyrimidine-2,4(1H,3H)-dione 15c. To a solution of compound 15b (10 g, 45.45 mmol) in DCM (100 mL) was added BBr₃ (227 mL, 227.2 mmol) dropwise over 30 min. After stirred for 2 h, the reaction was quenched with H₂O (100 mL) and the reaction mixture was extracted with EtOAc (150 mL×2). The combined organic layers were dried over anhydrous Na₂SO₄, filtered, and concentrated to afford 15c (9 g) in 96% yield. MS (ESI) m/z: 207.2 [M+H]⁺.

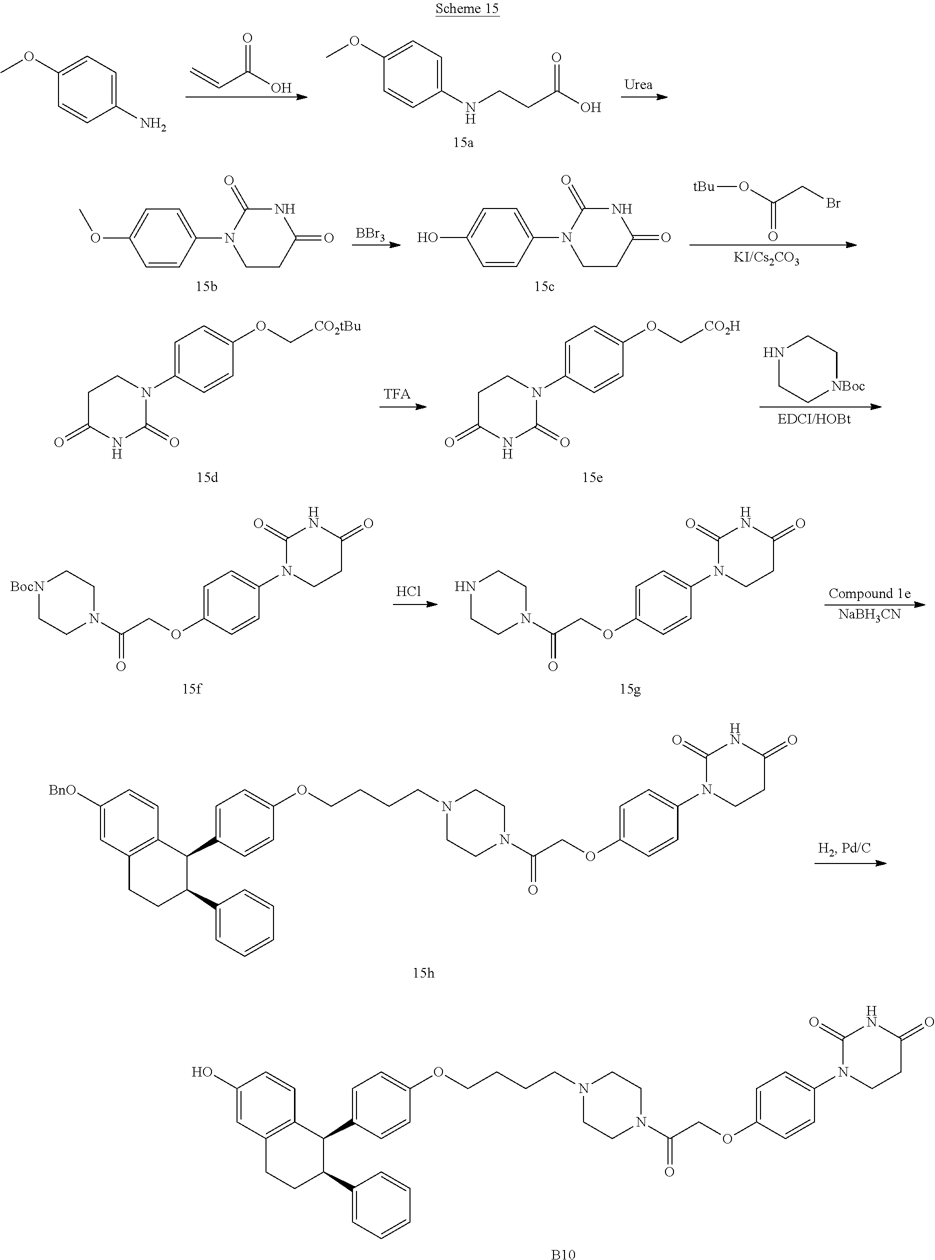

Tert-butyl 2-(4-(2,4-dioxotetrahydropyrimidin-1(2H)-yl)phenoxy)acetate 15d. To a solution of compound 15c (1 g, 4.85 mmol) in DMF (10 mL) were added Cs₂CO₃ (1.9 g, 5.82 mmol), KI (403 mg, 2.43 mmol), and tert-butyl 2-bromoacetate (946 mg, 4.85 mmol). After stirred for 8 h, the reaction was quenched with H₂O (20 mL) and the reaction mixture was extracted with EtOAc (30 mL×2). The combined organic layers were dried over anhydrous Na₂SO₄, filtered, and concentrated to yield a crude product, which was purified by silica gel column chromatography eluting with EtOAc in PE to afford compound 15d (330 mg) in 21% yield. MS (ESI) m/z: 265.1 [M-55]⁺.

2-(4-(2,4-Dioxotetrahydropyrimidin-1(2H)-yl)phenoxy)acetic acid 15e. To a solution of compound 15d (330 mg, 1.03 mmol) in DCM (2 mL) was added TFA (3 mL). After stirred overnight, the reaction mixture was concentrated and co-evaporated with toluene to afford compound 15e (210 mg) in 77% yield. ¹H NMR (400 MHz, DMSO-d₆) δ 10.29 (s, 1H), 7.23 (d, J=9.2 Hz, 2H), 6.92 (d, J=9.2 Hz, 2H), 4.68 (s, 2H), 3.72 (t, J=6.8 Hz, 2H), 2.69 (t, J=6.8 Hz, 2H); MS (ESI) m/z: 265.1 [M+H]⁺.

Tert-butyl 4-(2-(4-(2,4-dioxotetrahydropyrimidin-1(2H)-yl)phenoxy)acetyl)-piperazine-1-carboxylate 15f. To a solution of compound 15e (150 mg, 0.57 mmol) and tert-butyl piperazine-1-carboxylate (106 mg, 0.57 mmol) in DMF (4 mL) were added HOBt (115 mg, 0.86 mmol), EDCI·HCl (165 mg, 0.86 mmol) and DIEA (220 mg, 1.71 mmol). After stirred for 12 h, the reaction mixture was diluted with H₂O (10 mL) and extracted with EtOAc (15 mL×2). The combined organic layers were dried over anhydrous Na₂SO₄, filtered, and concentrated to yield a crude product, which was purified by silica gel column chromatography eluting with MeOH in DCM to afford compound 15f (172 mg) in 70% yield. MS (ESI) m/z: 433.2 [M+H]⁺.

1-(4-(2-Oxo-2-(piperazin-1-yl)ethoxy)phenyl)dihydropyrimidine-2,4(1H,3H)-dione 15g. To a solution of compound 15f (100 mg, 0.23 mmol) in DCM (8 mL) was added HCl in EtOAc (2 mL) dropwise. After stirred for 2 h, the reaction mixture was concentrated to afford compound 15g (80 mg, crude). MS (ESI) m/z: 333.1 [M+H]⁺.

1-(4-(2-(4-(4-(4-((1R,2S)-6-(Benzyloxy)-2-phenyl-1,2,3,4-tetrahydronaphthalen-1-yl)phenoxy)butyl)piperazin-1-yl)-2-oxoethoxy)phenyl)dihydropyrimidine-2,4(1H,3H)-dione 15h. To a solution of 4-(4-((1R,2S)-6-(benzyloxy)-2-phenyl-1,2,3,4-tetrahydronaphthalen-1-yl)phenoxy)butanal 1e (120 mg, 0.252 mmol) in DCM (4 mL) and MeOH (0.4 mL) was added compound 15g (84 mg, 0.252 mmol). After the mixture was stirred for 30 min, NaBH₃CN (32 mg, 0.504 mmol) was added. The reaction mixture was stirred for 12 h and then concentrated to yield a crude product, which was purified by silica gel column chromatography eluting with MeOH in DCM to afford compound 15h (141 mg) in 71% yield. MS (ESI) m/z: 793.3 [M+H]⁺.

1-(4-(2-(4-(4-(4-((1R,2S)-6-Hydroxy-2-phenyl-1,2,3,4-tetrahydronaphthalen-1 yl)phenoxy)butyl)piperazin-1-yl)-2-oxoethoxy)phenyl)dihydropyrimidine-2,4(1H,3H)-dione B10. To a solution of compound 15h (141 mg, 0.178 mmol) in MeOH (4 mL) and THE (4 mL) was added Pd/C (110 mg). After stirred under H₂ for 12 h, the reaction mixture was filtered and concentrated to yield a crude product, which was purified by reverse phase prep-HPLC to afford compound B10 (54.1 mg) in 43% yield. ¹H NMR (400 MHz, DMSO-d₆) δ 10.29 (s, 1H), 10.19 (s, 1H), 9.12 (s, 1H), 7.22 (d, J=8.8 Hz, 2H), 7.17-7.11 (m, 3H), 6.94 (d, J=9.2 Hz, 2H), 6.83 (d, J=6.8 Hz, 2H), 6.64-6.61 (m, 2H), 6.54 (d, J=8.8 Hz, 2H), 6.49-6.47 (m, 1H), 6.27 (d, J=8.8 Hz, 2H), 4.88 (d, J=8.8 Hz, 2H), 4.38 (d, J=16.4 Hz, 1H), 4.18 (d, J=4.8 Hz, 1H), 4.01 (d, J=13.6 Hz, 1H), 3.84 (t, J=11.6 Hz, 2H), 3.71 (t, J=6.8 Hz, 2H), 3.49 (d, J=11.2 Hz, 3H), 3.11-2.93 (m, 8H), 2.69 (t, J=6.8 Hz, 2H), 2.14-1.95 (m, 2H), 1.79-1.67 (m, 6H); MS (ESI) m/z: 703.3 [M+H]⁺.

### Example 16

### Preparation of 1-(3-((4-(((4-(4-((1S,2R)-6-hydroxy-2-phenyl-1,2,3,4-tetrahydronaphthalen-1-yl)phenoxy)butyl)amino)methyl)benzyl)oxy)phenyl)dihydropyrimidine-2,4(1H,3H)-dione B11

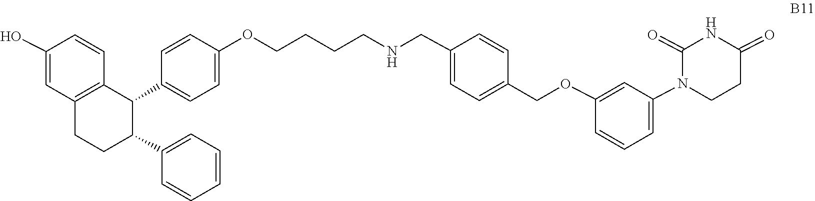 [SMILES: O=C1CCN(c2cccc(OCc3ccc(CNCCCCOc4ccc([C@H]5c6ccc(O)cc6CC[C@H]5c5ccccc5)cc4)cc3)c2)C(=O)N1]

Compound B11 is prepared as shown in Scheme 16.

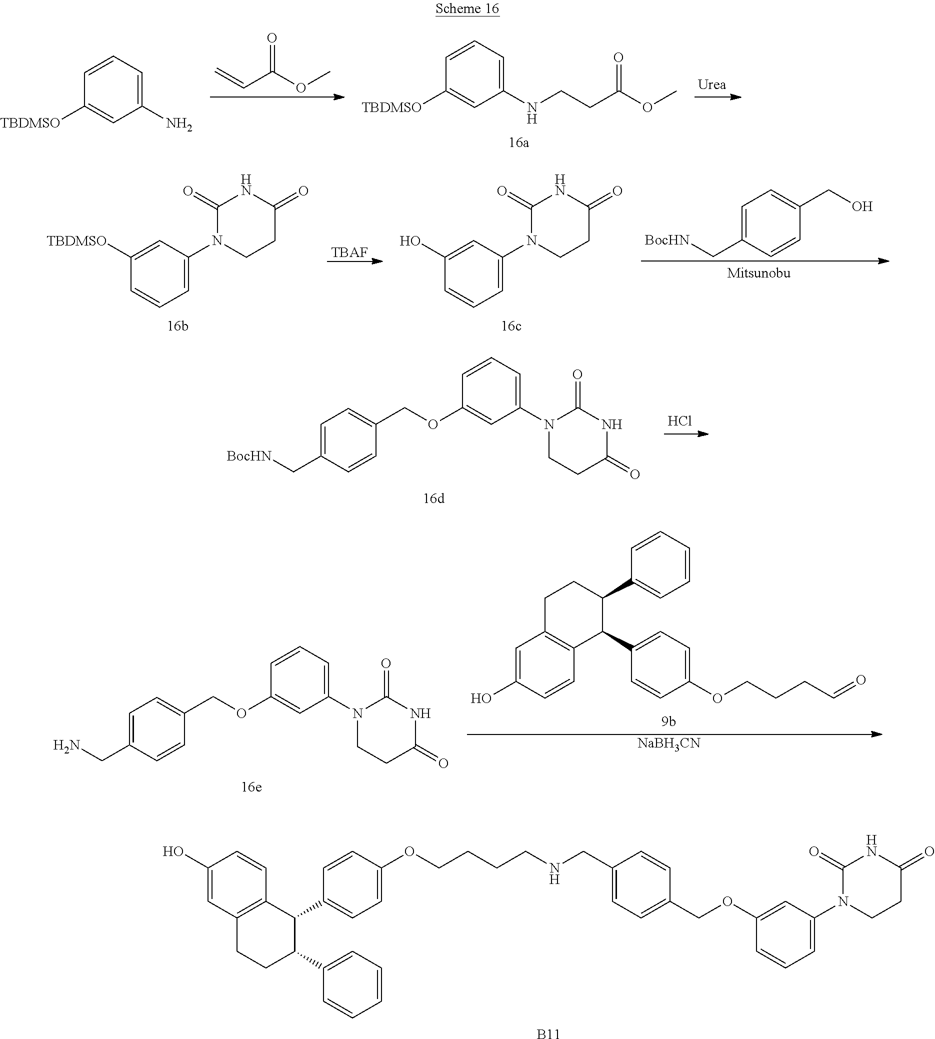

### Example 17

### Preparation of 1-(4-((4-(((4-(4-((1S,2R)-6-hydroxy-2-phenyl-1,2,3,4-tetrahydronaphthalen-1-yl)phenoxy)butyl)amino)methyl)benzyl)oxy)phenyl)dihydropyrimidine-2,4(1H,3H)-dione B12

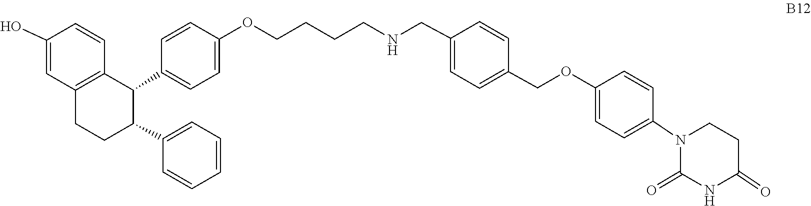 [SMILES: O=C1CCN(c2ccc(OCc3ccc(CNCCCCOc4ccc([C@H]5c6ccc(O)cc6CC[C@H]5c5ccccc5)cc4)cc3)cc2)C(=O)N1]

Compound B12 is prepared as shown in Scheme 17.

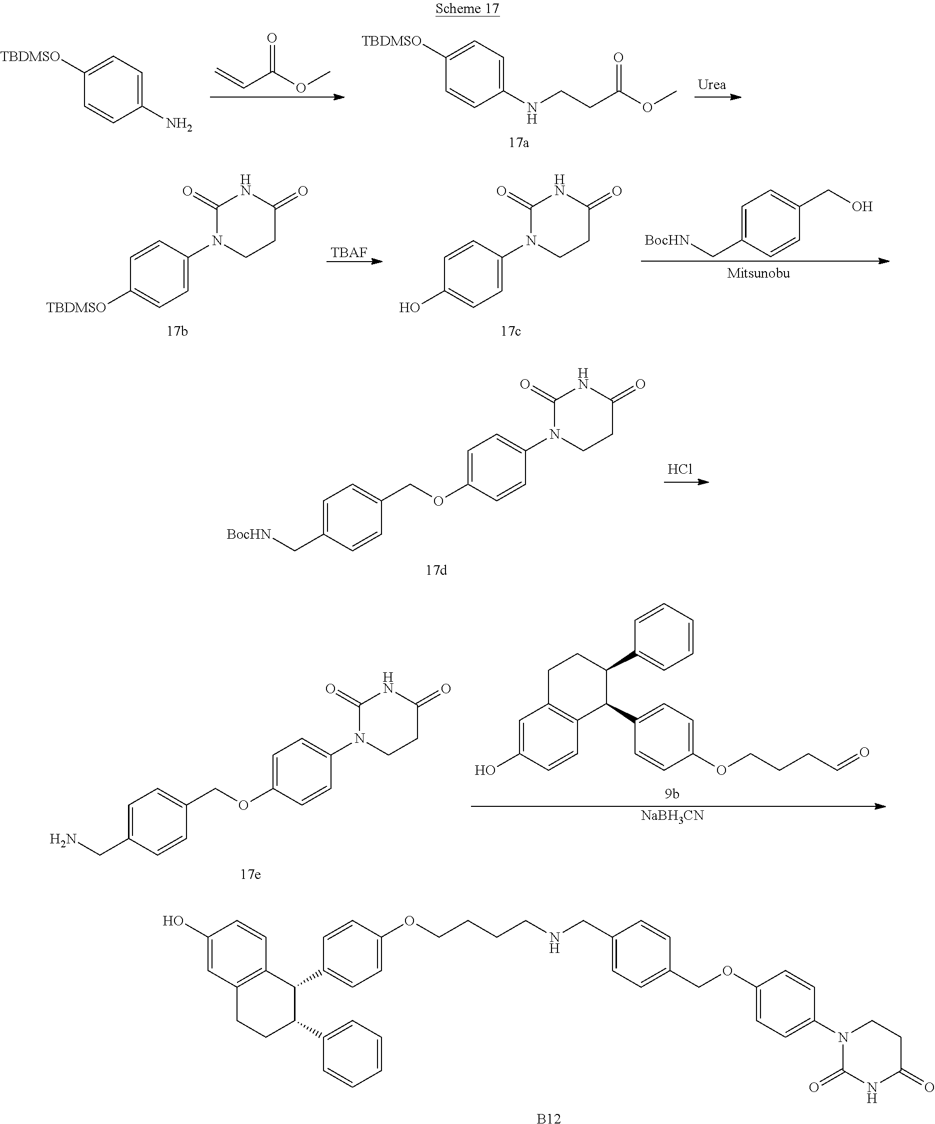

### Example 18

### Preparation of 3-((3-(4-((1-(4-((1R,2S)-6-hydroxy-2-phenyl-1,2,3,4-tetrahydronaphthalen-1-yl)phenyl)piperidin-4-yl)methyl)piperazin-1-yl)phenyl)amino)piperidine-2,6-dione B13

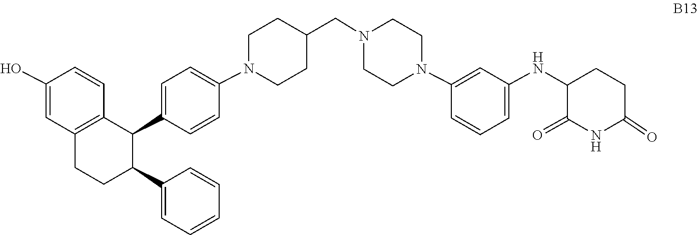 [SMILES: O=C1CCC(Nc2cccc(N3CCN(CC4CCN(c5ccc([C@@H]6c7ccc(O)cc7CC[C@@H]6c6ccccc6)cc5)CC4)CC3)c2)C(=O)N1]

Compound B13 was prepared as shown in Scheme 18.

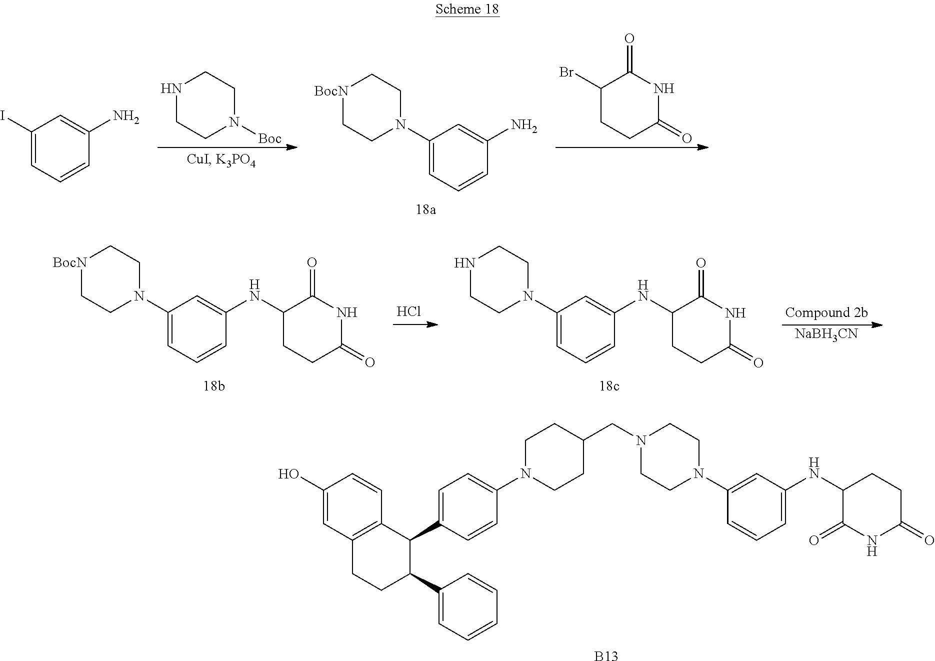

Tert-butyl 4-(3-aminophenyl)piperazine-1-carboxylate 18a. To a solution of 3-iodoaniline (5 g, 22.8 mmol) in 2-propanol (18 mL) were added tert-butyl piperazine-1-carboxylate (5.1 g, 27.4 mmol), CuI (217 mg, 1.1 mmol), K₃PO₄ (9.7 g, 45.6 mmol), and ethylene glycol (2 mL). After stirred at 80° C. for 2 d, the reaction mixture was diluted with H₂O (10 mL) and extracted with EtOAc (15 mL×3). The combined organic layers were dried over anhydrous Na₂SO₄, filtered, and concentrated to yield a crude product, which was purified by silica gel column chromatography eluting with EtOAc in PE to afford compound 18a (1.8 g) in 29% yield. MS (ESI) m z 278.2 [M+H]⁺.

Tert-butyl 4-(3-((2,6-dioxopiperidin-3-yl)amino)phenyl)piperazine-1-carboxylate 18b. To a solution of compound 18a (1.8 g, 6.5 mmol) in DMF (20 mL) were added tert-butyl (3-aminophenyl)carbamate (1.24 g, 6.5 mmol) and NaHCO₃ (540 mg, 6.5 mmol). After stirred at 80° C. overnight, the reaction mixture was diluted with H₂O (20 mL) and extracted with EtOAc (30 mL×2). The combined organic layers were dried over anhydrous Na₂SO₄, filtered, and concentrated to yield a crude product, which was purified by silica gel column chromatography eluting with MeOH in DCM to afford compound 18b (1.7 g) in 67% yield. MS (ESI) m/z: 389.2 [M+H]⁺.

3-((3-(Piperazin-1-yl)phenyl)amino)piperidine-2,6-dione 18c. To a solution of compound 18b (150 mg, 0.39 mmol) in dimethoxyethane (10 mL) was added HCl in EtOAc (3 mL). After stirred for 2 h, the reaction mixture was concentrated to afford compound 18c (113 mg) as a crude product, which was used directly in the next step without further purification. MS (ESI) m/z: 289.2 [M+H]⁺.

3-((3-(4-((1-(4-((1R,2S)-6-Hydroxy-2-phenyl-1,2,3,4-tetrahydronaphthalen-1-yl)-phenyl)piperidin-4-yl)methyl)piperazin-1-yl)phenyl)amino)piperidine-2,6-dione B13. To a solution of 1-(4-((1R,2S)-6-hydroxy-2-phenyl-1,2,3,4-tetrahydronaphthalen-1-yl)phenyl)-piperidine-4-carbaldehyde 2b (82 mg, 0.20 mmol) in DMF (3 mL) were added compound 18c (58 mg, 0.20 mmol), DIEA (26 mg, 0.20 mmol), NaBH₃CN (37.8 mg, 0.60 mmol), and AcOH (1 drop). After stirred overnight, the reaction mixture was concentrated to yield a crude product, which was purified by reverse phase prep-HPLC to afford compound B13 (34.2 mg) in 25% yield. ¹H NMR (400 MHz, DMSO-d₆) δ 10.81 (s, 1H), 10.73 (s, 1H), 7.42 (s, 2H), 7.18-7.11 (m, 3H), 6.98 (t, J=8.4 Hz, 1H), 6.84-6.82 (m, 2H), 6.67-6.65 (m, 2H), 6.53-6.51 (m, 3H), 6.35 (s, 1H), 6.29-6.24 (m, 2H), 4.38-4.31 (m, 2H), 3.77-3.72 (m, 4H), 3.69-3.60 (m, 2H), 3.57-3.49 (m, 2H), 3.47-3.41 (m, 2H), 3.37-3.27 (m, 2H), 3.18-3.08 (m, 4H), 3.01-2.90 (m, 2H), 2.79-2.70 (m, 1H), 2.61-2.56 (m, 1H), 2.27-2.11 (m, 1H), 2.03-1.92 (m, 4H), 1.74-1.24 (m, 4H); MS (ESI) m/z: 684.5 [M+H]⁺.

### Example 19

### Preparation of 3-((3-((1-((1-(4-((1R,2S)-6-hydroxy-2-phenyl-1,2,3,4-tetrahydronaphthalen-1-yl)phenyl)piperidin-4-yl)methyl)piperidin-4-yl)amino)phenyl)amino)piperidine-2,6-dione B14

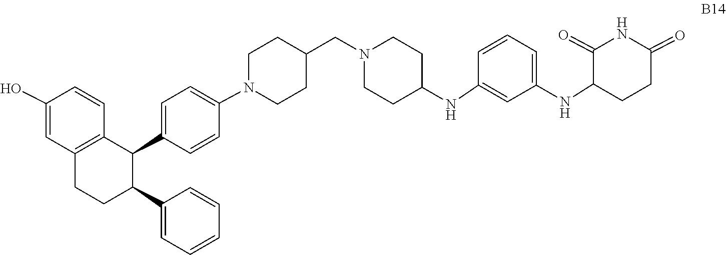 [SMILES: O=C1CCC(Nc2cccc(NC3CCN(CC4CCN(c5ccc([C@@H]6c7ccc(O)cc7CC[C@@H]6c6ccccc6)cc5)CC4)CC3)c2)C(=O)N1]

Compound B14 was prepared as shown in Scheme 19.

Tert-butyl (3-((2,6-dioxopiperidin-3-yl)amino)phenyl)carbamate 19a. To a solution of 3-bromopiperidine-2,6-dione (4.5 g, 23.4 mmol) in DMF (27 mL) were added tert-butyl (3-aminophenyl)carbamate (4.9 g, 23.4 mmol) and NaHCO₃ (2 g, 23.4 mmol). After stirred at 80° C. overnight, the reaction mixture was diluted with water (10 mL) and extracted with EtOAc (20 mL×2). The combined organic layers were dried over anhydrous Na₂SO₄, filtered, and concentrated to yield a crude product, which was purified by silica gel column chromatography eluting with MeOH in DCM to afford compound 19a (6.6 g) in 88% yield. MS (ESI) m/z: 320.1 [M+H]⁺.

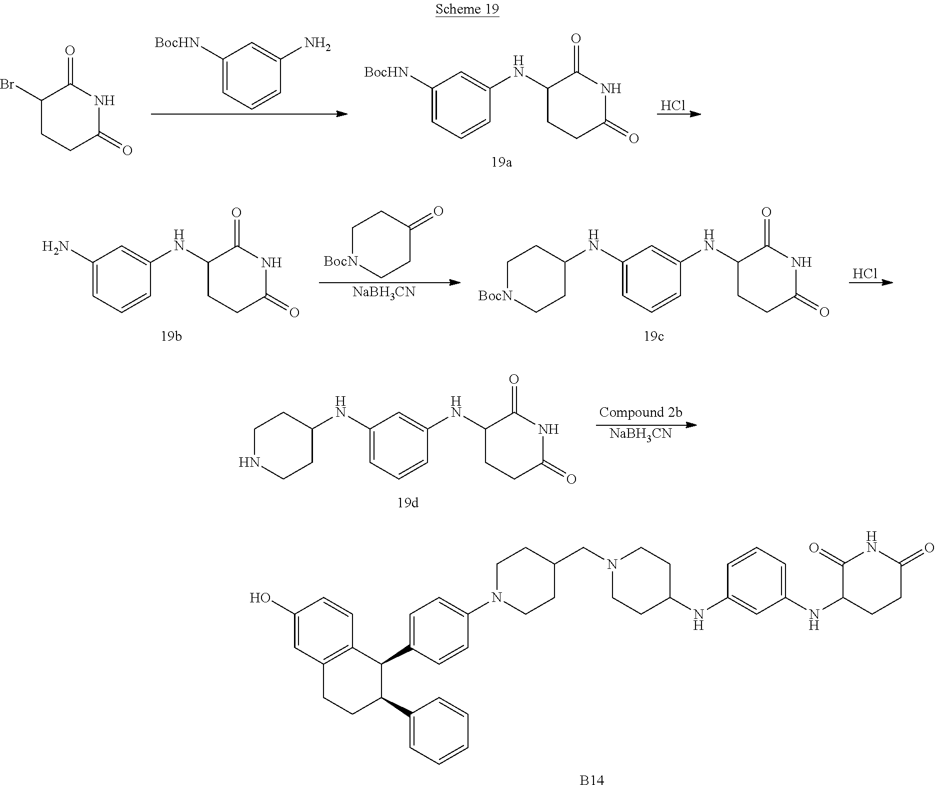

3-((3-Aminophenyl)amino)piperidine-2,6-dione 19b. To a solution of compound 19a (6.6 g, 20.5 mmol) in dimethoxyethane (40 mL) was added HCl in EtOAc (20 mL). After stirred for 2 h, the reaction mixture was concentrated to afford compound 19b (4.5 g) in a quantitative yield. MS (ESI) m/z: 220.1 [M+H]⁺.

Tert-butyl 4-((3-((2,6-dioxopiperidin-3-yl)amino)phenyl)amino)piperidine-1-carboxylate 19c. To a solution of compound 19b (4.5 g, 20.5 mmol) in DMF (45 mL) were added tert-butyl 4-oxopiperidine-1-carboxylate (7.1 g, 35.4 mmol), NaBH₃CN (3.8 g, 61.5 mmol), and AcOH (1 drop). After the reaction was complete, the reaction mixture was diluted with water (10 mL) and extracted with EtOAc (40 mL×2). The combined organic layers were dried over anhydrous Na₂SO₄, filtered, and concentrated to yield a crude product, which was purified by silica gel column chromatography eluting with MeOH in DCM to afford compound 19c (4.5 g) in 54% yield. MS (ESI) m/z: 403.2 [M+H]⁺.

3-((3-(Piperidin-4-ylamino)phenyl)amino)piperidine-2,6-dione 19d. To a solution of compound 19c (100 mg, 0.248 mmol) in DCM (15 mL) was added HCl in EtOAc (3 mL). After stirred for 2 h, the reaction mixture was concentrated to afford compound 19d (80 mg) as a crude product, which was used directly in the next step without further purification. MS (ESI) m/z: 303.2 [M+H]⁺.

3-((3-((1-((1-(4-((1R,2S)-6-Hydroxy-2-phenyl-1,2,3,4-tetrahydronaphthalen-1-yl)phenyl)piperidin-4-yl)methyl)piperidin-4-yl)amino)phenyl)amino)piperidine-2,6-dione B14. To a solution of 1-(4-((1R,2S)-6-hydroxy-2-phenyl-1,2,3,4-tetrahydronaphthalen-1-yl)phenyl)-piperidine-4-carbaldehyde 2b (89 mg, 0.22 mmol) in DMF (3 mL) were added compound 19d (66 mg, 0.22 mmol), DIEA (29 mg, 0.22 mmol), NaBH₃CN (138 mg, 0.66 mmol), and AcOH (1 drop). After stirred overnight, the reaction mixture was concentrated to yield a crude product, which was purified by reverse phase prep-HPLC to afford B14 (68 mg) in 44% yield. ¹H NMR (400 MHz, DMSO-d₆) δ 10.76 (s, 1H), 9.11 (s, 1H), 8.15 (s, 1H), 7.16-7.09 (m, 3H), 6.83 (d, J=6.8 Hz, 2H), 6.76 (t, J=8.0 Hz, 1H), 6.64 (d, J=8.0 Hz, 1H), 6.59 (s, 1H), 6.53-6.47 (m, 3H), 6.19 (d, J=8.4 Hz, 2H), 5.91-5.86 (m, 3H), 5.45 (d, J=7.2 Hz, 1H), 5.06 (s, 1H), 4.21-4.12 (m, 2H), 3.51-3.48 (m, 2H), 3.30-3.27 (m, 3H), 3.14-3.07 (m, 1H), 3.01-2.90 (m, 2H), 2.84-2.81 (m, 2H), 2.76-2.67 (m, 1H), 2.59-2.54 (m, 1H), 2.19-2.02 (m, 6H), 1.88-1.82 (m, 3H), 1.73-1.70 (m, 3H), 1.63-1.54 (m, 1H), 1.39-1.31 (m, 2H), 1.16-1.08 (m, 2H); MS (ESI) m/z: 698.6 [M+H]⁺.

### Example 20

### Preparation of 3-((3-(2-(4-((1-(4-((1R,2S)-6-hydroxy-2-phenyl-1,2,3,4-tetrahydronaphthalen-1-yl)phenyl)piperidin-4-yl)methyl)piperazin-1-yl)-2-oxoethoxy)phenyl)amino)piperidine-2,6-dione B16

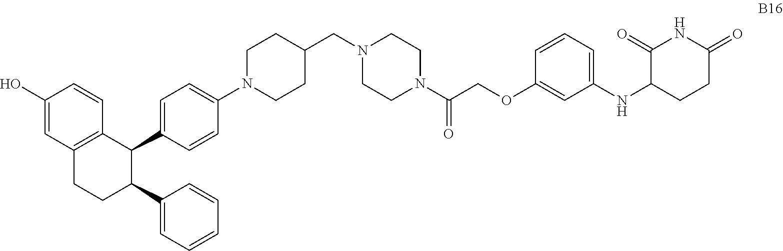 [SMILES: O=C1CCC(Nc2cccc(OCC(=O)N3CCN(CC4CCN(c5ccc([C@@H]6c7ccc(O)cc7CC[C@@H]6c6ccccc6)cc5)CC4)CC3)c2)C(=O)N1]

Compound B16 was prepared as shown in Scheme 20.

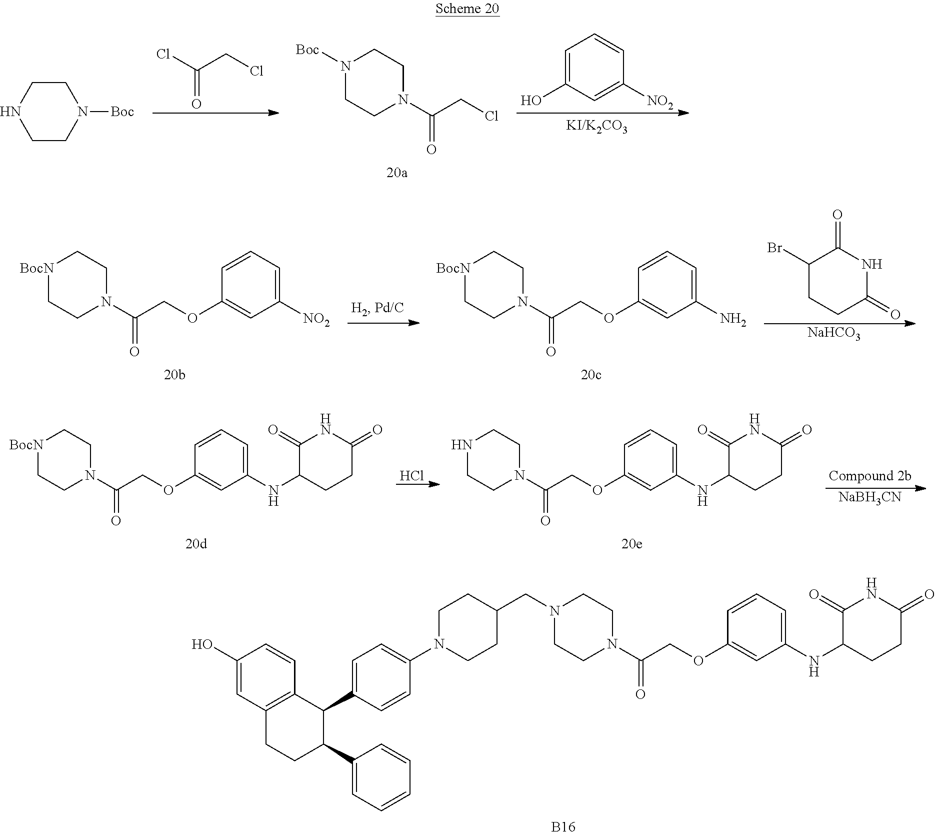

Tert-butyl 4-(2-chloroacetyl)piperazine-1-carboxylate 20a. To a solution of tert-butyl piperazine-1-carboxylate (5 g, 26.9 mmol) and TEA (5.4 g, 53.8 mmol) in DCM (50 mL) was added 2-chloroacetyl chloride (3.6 g, 32.3 mmol) dropwise at 0° C. After stirred at 25° C. for 2 h, the reaction mixture was concentrated to yield a crude product, which was purified by silica gel column chromatography eluting with MeOH in DCM to afford compound 20a (6.5 g) in 92% yield. MS (ESI) m/z: 263.1 [M+H]⁺.

Tert-butyl 4-(2-(3-nitrophenoxy)acetyl)piperazine-1-carboxylate 20b. To a solution of compound 20a (3 g, 11.5 mmol) in DMF (30 mL) were added 3-nitrophenol (1.91 g, 13.7 mmol), K₂CO₃ (3.16 g, 22.9 mmol), and KI (950 mg, 5.7 mmol). After stirred at 50° C. overnight, the reaction mixture was diluted with H₂O (20 mL) and extracted with EtOAc (50 mL×2). The combined organic layers were dried over anhydrous Na₂SO₄, filtered, and concentrated to yield a crude product, which was purified by silica gel column chromatography eluting with EtOAc in PE to afford compound 20b (3.9 g). MS (ESI) m/z: 366.1 [M+H]⁺.

Tert-butyl 4-(2-(3-aminophenoxy)acetyl)piperazine-1-carboxylate 20c. To a solution of compound 20b (3.9 g, 10.7 mmol) in MeOH (30 mL) was added Pd/C (500 mg). After stirred under H₂ for 1 h, the reaction mixture was concentrated to afford compound 20c (3.4 g) as a crude product, which was used directly in the next step without further purification. MS (ESI) m/z: 336.2 [M+H]⁺.

Tert-butyl 4-(2-(3-((2,6-dioxopiperidin-3-yl)amino)phenoxy)acetyl)piperazine-1-carboxylate 20d. To a solution of compound 20c (3.4 g, 10.1 mmol) in DMF (30 mL) were added 3-bromopiperidine-2,6-dione (2.3 g, 12.2 mmol) and Na₂CO₃ (1.7 g, 20.3 mmol). After stirred at 80° C. overnight, the reaction mixture was diluted with H₂O (10 mL) and extracted with EtOAc (30 mL×2). The combined organic layers were washed with H₂O (30 mL), dried over anhydrous Na₂SO₄, filtered, and concentrated to yield a crude product, which was purified by silica gel column chromatography eluting with EtOAc in PE to afford compound 20d (2.1 g) in 67% yield. MS (ESI) m/z: 447.2 [M+H]⁺.

3-((3-(2-Oxo-2-(piperazin-1-yl)ethoxy)phenyl)amino)piperidine-2,6-dione 20e. To a solution of compound 20d (49 mg, 0.109 mmol) in DCM (4 mL) was added HCl in EtOAc (1 mL). After stirred for 1 h, the reaction mixture was concentrated to afford compound 20e as a crude product, which was used directly in the next step without further purification. MS (ESI) m/z: 347.2 [M+H]⁺.

3-((3-(2-(4-((1-(4-((1R,2S)-6-Hydroxy-2-phenyl-1,2,3,4-tetrahydronaphthalen-1-yl)phenyl)piperidin-4-yl)methyl)piperazin-1-yl)-2-oxoethoxy)phenyl)amino)piperidine-2,6-dione B16. To a solution of 1-(4-((1R,2S)-6-hydroxy-2-phenyl-1,2,3,4-tetrahydronaphthalen-1-yl)phenyl)piperidine-4-carbaldehyde 2b (45 mg, 0.109 mmol) in DCM (4 mL) were added DIEA (1 drop), compound 20e (38 mg, 0.109 mmol), AcOH (1 drop), and NaBH₃CN (14 mg, 0.218 mmol). After stirred overnight, the reaction mixture was concentrated to yield a crude product, which was purified by reverse phase prep-HPLC to afford compound B16 (34.2 mg) in 75% yield. ¹H NMR (DMSO-d₆, 400 MHz) δ 10.77 (s, 1H), 9.08 (s, 1H), 8.17 (s, 1H), 7.16-7.10 (m, 3H), 6.95 (t, J=8.0 Hz, 1H), 6.82 (d, J=6.0 Hz, 2H), 6.63 (d, J=8.4 Hz, 1H), 6.59 (d, J=2.0 Hz, 1H), 6.52 (d, J=2 Hz, 2H), 6.47 (dd, J=2.4, 8.4 Hz, 1H), 6.28 (dd, J=1.6, 8.0 Hz, 1H), 6.23 (t, J=2.0 Hz, 1H), 6.19 (d, J=8.8 Hz, 2H), 6.13 (dd, J=2.4, 8.4 Hz, 1H), 5.83 (d, J=7.6 Hz, 1H), 4.66 (s, 2H), 4.32-4.26 (m, 1H), 4.12 (d, J=4.0 Hz, 1H), 3.50-3.43 (m, 6H), 3.26-3.25 (m, 1H), 2.99-2.90 (m, 2H), 3.26-3.25 (m, 2H), 2.60-2.53 (m, 2H), 2.45-2.43 (m, 1H), 2.35-2.29 (m, 4H), 2.15-2.08 (m, 3H), 1.90-1.79 (m, 1H), 1.79-1.45 (m, 4H), 1.13-1.11 (m, 2H); MS (ESI) m/z: 742.3 [M+H]⁺.

### Example 21

### Preparation of 3-((3-((4-(((4-(4-((1S,2R)-6-hydroxy-2-phenyl-1,2,3,4-tetrahydronaphthalen-1-yl)phenoxy)butyl)amino)methyl)benzyl)oxy)phenyl)amino)piperidine-2,6-dione B17

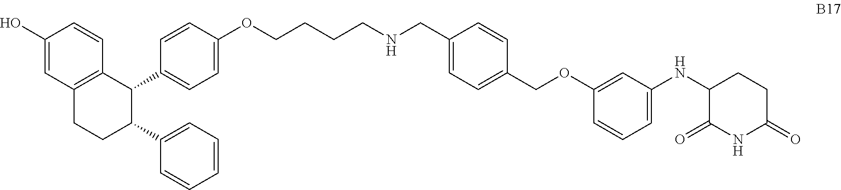 [SMILES: O=C1CCC(Nc2cccc(OCc3ccc(CNCCCCOc4ccc([C@H]5c6ccc(O)cc6CC[C@H]5c5ccccc5)cc4)cc3)c2)C(=O)N1]

Compound B17 is prepared as shown in Scheme 21.

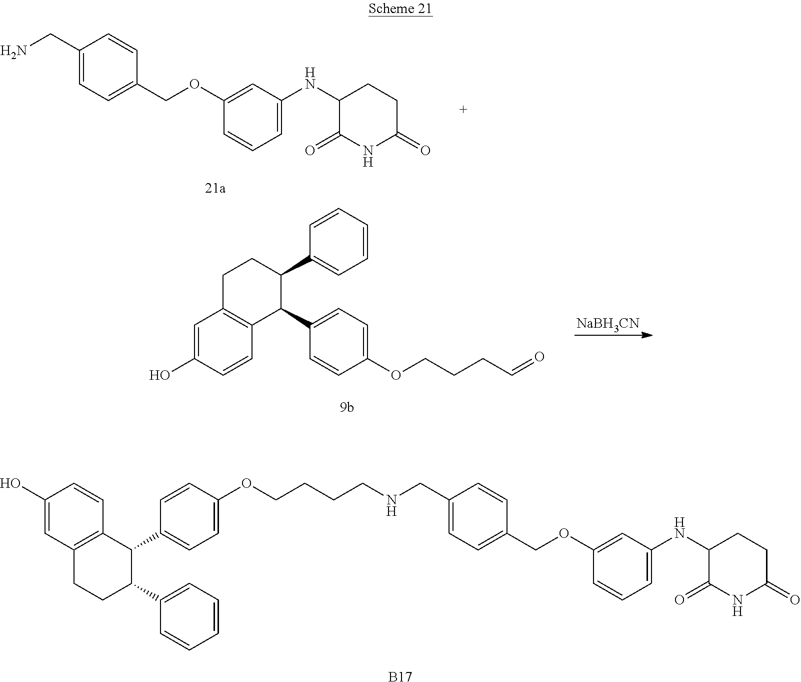 [SMILES: NCc1ccc(COc2cccc(NC3CCC(=O)NC3=O)c2)cc1 ; O=CCCCOc1ccc([C@H]2c3ccc(O)cc3CC[C@H]2c2ccccc2)cc1 ; "NaBH3CN" ; O=C1CCC(Nc2cccc(OCc3ccc(CNCCCCOc4ccc([C@H]5c6ccc(O)cc6CC[C@H]5c5ccccc5)cc4)cc3)c2)C(=O)N1 ; "B17"]

### Example 22

### Preparation of N-(3-((2,6-dioxopiperidin-3-yl)amino)phenyl)-2-(4-((1-(4-((1R,2S)-6-hydroxy-2-phenyl-1,2,3,4-tetrahydronaphthalen-1-yl)phenyl)piperidin-4-yl)methyl)piperazin-1-yl)-acetamide B19

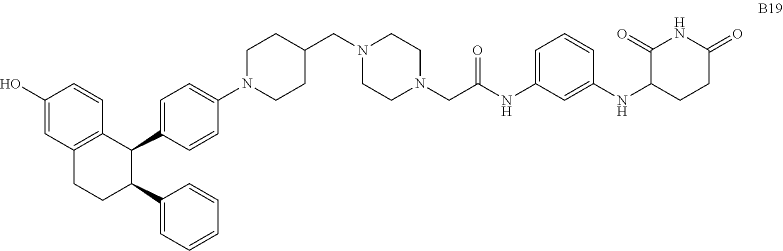 [SMILES: O=C1CCC(Nc2cccc(NC(=O)CN3CCN(CC4CCN(c5ccc([C@@H]6c7ccc(O)cc7CC[C@@H]6c6ccccc6)cc5)CC4)CC3)c2)C(=O)N1]

Compound B19 was prepared as shown in Scheme 22.

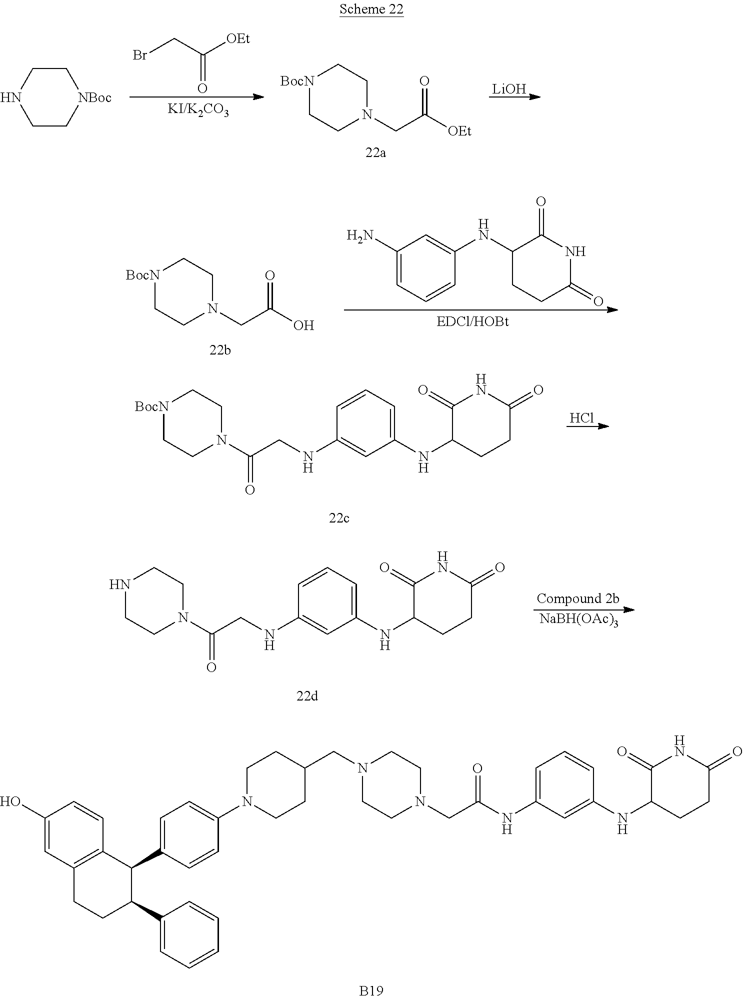

Tert-butyl 4-(2-ethoxy-2-oxoethyl)piperazine-1-carboxylate 22a. To a solution of tert-butyl piperazine-1-carboxylate (5 g, 26.9 mmol) and ethyl 2-bromoacetate (5.4 g, 32.2 mmol) in DMF (50 mL) were added K₂CO₃ (7.4 g, 53.7 mmol) and KI (4.4 g, 26.88 mmol). After stirred at 25° C. overnight, the reaction mixture was extracted with EtOAc (50 mL×3). The combined organic layers were concentrated to yield a crude product, which was purified by silica gel column chromatography eluting with MeOH in DCM to afford compound 22a (6.5 g) in 89% yield. MS (ESI) m/z: 273.2 [M+H]⁺.

2-(4-(Tert-butoxycarbonyl)piperazin-1-yl)acetic acid 22b. To a solution of compound 22a (3 g, 11.03 mmol) in THE (30 mL), MeOH (10 mL), and H₂O (10 mL) was added LiOH (926 mg, 22.6 mmol). After stirred at 25° C. overnight, the reaction mixture was concentrated and neutralized to pH 6 with 2N HCl. The resulting precipitates were collected by filtration and dried to afford compound 22b (2.2 g) as a crude product, which was used directly in the next step without further purification. MS (ESI) m/z: 245.1 [M+H]⁺.

Tert-butyl 4-(2-((3-((2,6-dioxopiperidin-3-yl)amino)phenyl)amino)-2-oxoethyl)-piperazine-1-carboxylate 22c. To a solution of compound 22b (1 g, 4.1 mmol) and 3-((3-aminophenyl)amino)piperidine-2,6-dione (987 mg, 4.51 mmol) in DMF (10 mL) were added HOBt (936 mg, 6.15 mmol), EDCI·HCl (1.2 g, 6.15 mmol), and DIEA (1.6 g, 12.3 mmol). After stirred overnight, the reaction mixture was diluted with H₂O (10 mL) and extracted with EtOAc (20 mL×3). The combined organic layers were dried over anhydrous Na₂SO₄, filtered, and concentrated to afford compound 22c (1 g) as a crude product, which was used directly in the next step without further purification. MS (ESI) m/z: 446.2 [M+H]⁺.

N-(3-((2,6-Dioxopiperidin-3-yl)amino)phenyl)-2-(piperazin-1-yl)acetamide 22d. To a solution of compound 22c (93 mg, 0.209 mmol) in DCM (4 mL) was added HCl in EtOAc (1 mL). After stirred for 1 h, the reaction mixture was concentrated to afford compound 22d as a crude product, which was used directly in the next step without further purification. MS (ESI) m/z: 346.1 [M+H]⁺.

N-(3-((2,6-Dioxopiperidn-3-yl)amino)phenyl)-2-(4-((1-(4-((1R,2S)-6-hydroxy-2-phenyl-1,2,3,4-tetrahydronaphthalen-1-yl)phenyl)piperidin-4-yl)methyl)piperazin-1-yl)-acetamide B19. To a solution of 1-(4-((1R,2S)-6-hydroxy-2-phenyl-1,2,3,4-tetrahydro-naphthalen-1-yl)phenyl)piperidine-4-carbaldehyde 2b (72 mg, 0.175 mmol) in DMF (4 mL) were added DIEA (1 drop), compound 22d (73 mg, 0.210 mmol), AcOH (1 drop), and NaBH(OAc)₃ (112 mg, 0.326 mmol). After stirred at room temperature overnight, the reaction mixture was concentrated to yield a crude product, which was purified by reverse phase prep-HPLC to afford compound B19 (29.6 mg) in 23% yield. ¹H NMR (400 MHz, DMSO-d₆) δ 10.76 (s, 1H), 9.36 (s, 1H), 9.08 (s, 1H), 8.16 (s, 1H), 7.16-7.10 (m, 3H), 7.00-6.96 (m, 2H), 6.83-6.79 (m, 3H), 6.63 (d, J=8.4 Hz, 1H), 6.60-6.59 (m, 1H), 6.51 (d, J=8.4 Hz, 2H), 6.47 (dd, J=2.4, 8.4 Hz, 1H), 6.38 (dd, J=1.6, 8.0 Hz, 1H), 6.19 (d, J=8.4 Hz, 2H), 5.86 (d, J=8.0 Hz, 1H), 4.28-4.21 (m, 1H), 4.12 (d, J=4.8 Hz, 1H), 3.50-3.47 (m, 2H), 3.31-3.13 (m, 4H), 3.05 (s, 2H), 2.96-2.90 (m, 2H), 2.77-2.56 (m, 1H), 2.44-2.32 (m, 9H), 2.14-2.07 (m, 5H), 1.72-1.69 (m, 3H), 1.15-1.07 (m, 2H); MS (ESI) m/z: 741.4 [M+H]⁺.

### Example 23

### Preparation of 3-((3-((2-(4-((1-(4-((1R,2S)-6-hydroxy-2-phenyl-1,2,3,4-tetrahydronaphthalen-1-yl)phenyl)piperidin-4-yl)methyl)piperazin-1-yl)-2-oxoethyl)amino)phenyl)amino)piperidine-2,6-dione B21

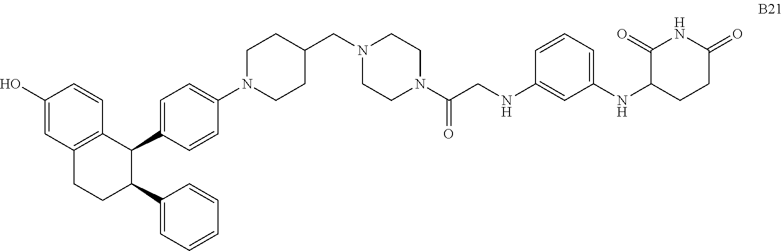 [SMILES: O=C1CCC(Nc2cccc(NCC(=O)N3CCN(CC4CCN(c5ccc([C@@H]6c7ccc(O)cc7CC[C@@H]6c6ccccc6)cc5)CC4)CC3)c2)C(=O)N1]

Compound B21 is prepared as shown in Scheme 23.

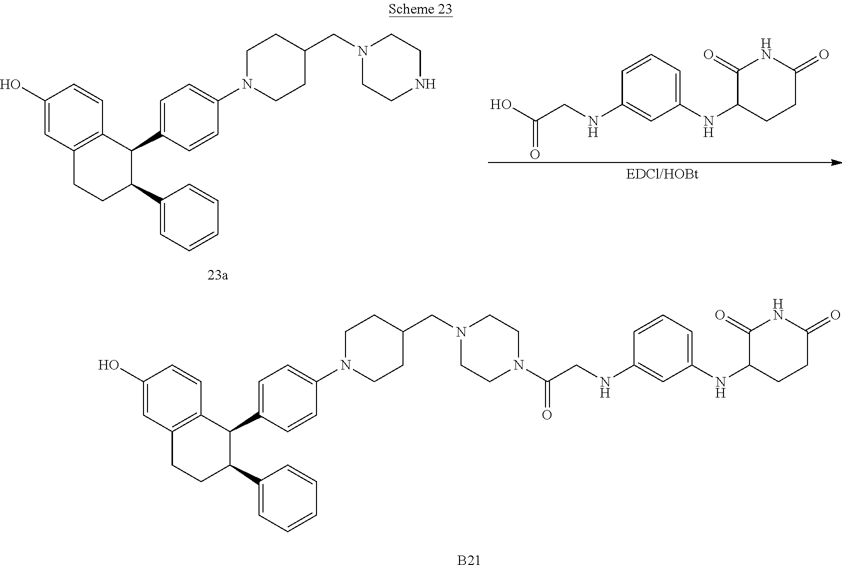

### Example 24

### Preparation of (2S,4R)-4-hydroxy-1-((S)-2-(2-(2-(4-(2-(4-((1R,2S)-6-hydroxy-2-phenyl-1,2,3,4-tetrahydronaphthalen-1-yl)phenoxy)ethyl)piperazin-1-yl)ethoxy)acetamido)-3,3-dimethyl-butanoyl)-N-(4-(4-methylthiazol-5-yl)benzyl)pyrrolidine-2-carboxamide C1

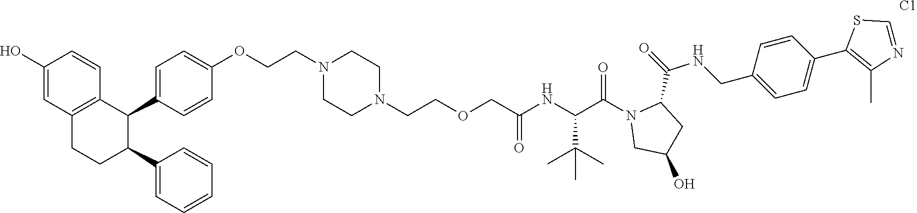

Compound C1 was prepared as shown in Scheme 24.

Tert-butyl 2-(4-((1R,2S)-6-(benzyloxy)-2-phenyl-1,2,3,4-tetrahydronaphthalen-1-yl)phenoxy)acetate 24b. To a solution of 4-((1R,2S)-6-(benzyloxy)-2-phenyl-1,2,3,4-tetrahydro-naphthalen-1-yl)phenol 24a (206 mg, 0.49 mmol) and tert-butyl 2-bromoacetate (198 mg, 1.01 mmol) in DMF (8 mL) were added K₂CO₃ (140 mg, 1.01 mmol) and KI (84 mg, 0.507 mmol). After stirred at 80° C. overnight, the reaction mixture was diluted with H₂O (10 mL) and extracted with EtOAc (20 mL×3). The combined organic layers were washed with sat. aqueous NaCl, dried over anhydrous Na₂SO₄, filtered, and concentrated to yield a crude product, which was purified by silica gel column chromatography eluting with EtOAc in PE to afford compound 24b (245 mg) in 96% yield. MS (ESI) m/z: 538.3 [M+NH₄]⁺.

2-(4-((1R,2S)-6-(Benzyloxy)-2-phenyl-1,2,3,4-tetrahydronaphthalen-1-yl)-phenoxy)acetic acid 24c. To a solution of compound 24b (245 mg, 0.471 mmol) in THE (4 mL), MeOH (1 mL), and H₂O (1 mL) was added LiOH (40 mg, 0.942 mmol). After stirred overnight, the reaction mixture was diluted with H₂O (5 mL) and then neutralized to pH 6 with 1N HCl. The resulting precipitates were collected by filtration and dried to afford compound 24c (194 mg) in 89% yield. MS (ESI) m/z: 482.2 [M+NH_{4]}⁺.

2-(4-((1R,2S)-6-(Benzyloxy)-2-phenyl-1,2,3,4-tetrahydronaphthalen-1-yl)-phenoxy)-N-methoxy-N-methylacetamide 24d. To a solution of compound 24c (401 mg, 0.86 mmol) and N,O-dimethylhydroxylamine hydrochloride (93 mg, 0.95 mmol) in DCM (8 mL) were added DIEA (223 mg, 1.73 mmol), DMAP (11 mg, 0.086 mmol), and EDCI (249 mg, 1.29 mmol). After stirred for 12 h, the reaction mixture was diluted with H₂O (20 mL) and extracted with EtOAc (20 mL×3). The combined organic layers were washed with sat. aqueous NaCl, dried over anhydrous Na₂SO₄, filtered, and concentrated to yield a crude product, which was purified by silica gel column chromatography eluting with EtOAc in PE to afford compound 24d (321 mg) in 74% yield. MS (ESI) m/z: 5082 [M+H]⁺.

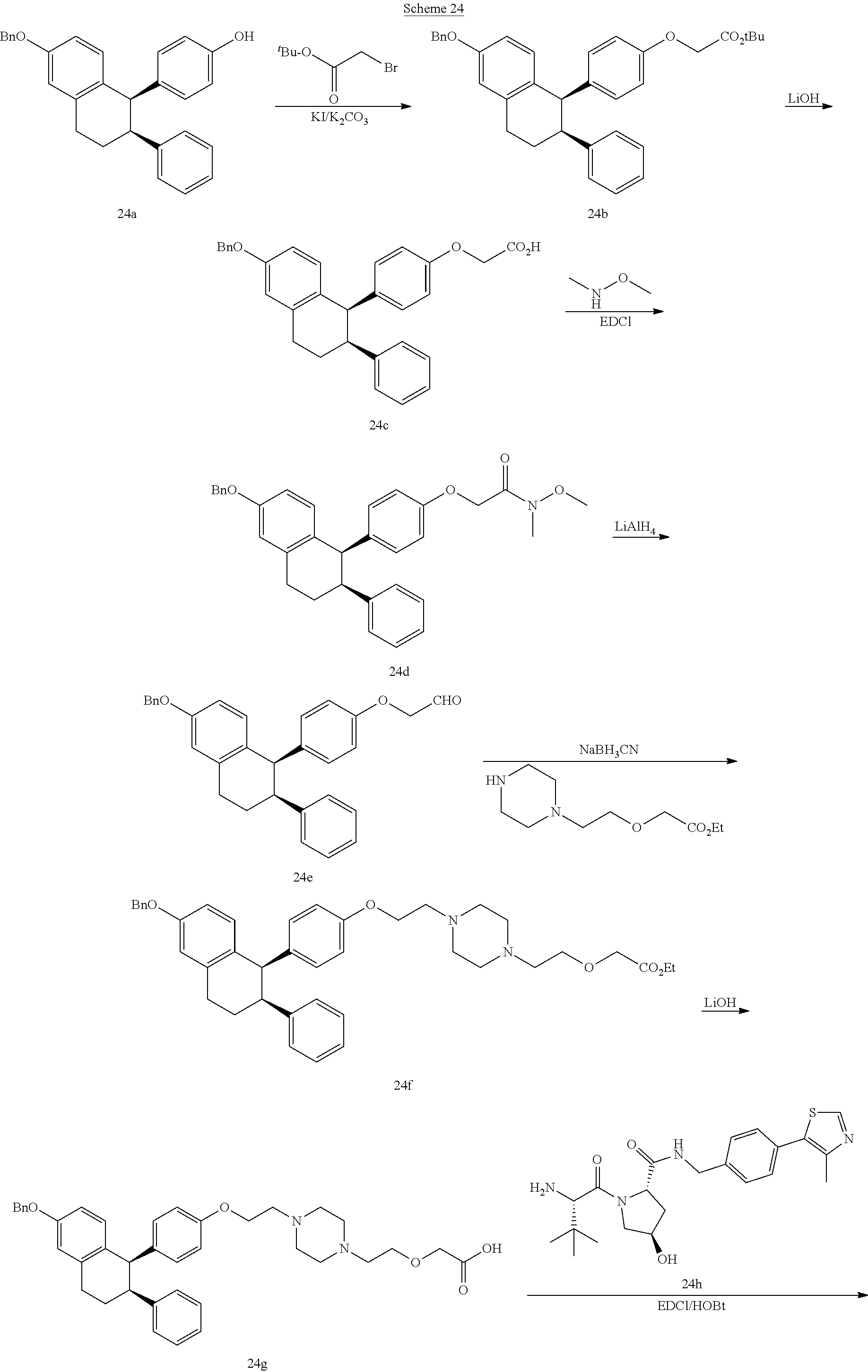

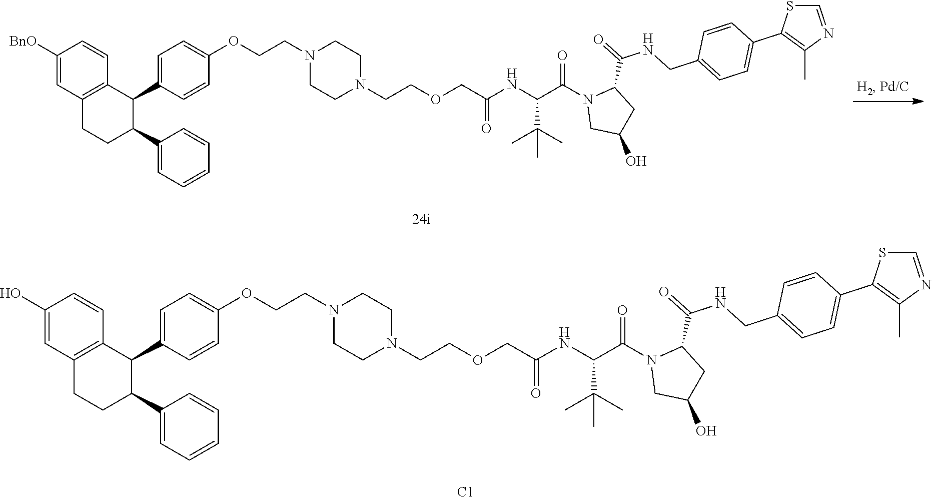 [SMILES: Cc1ncsc1-c1ccc(CNC(=O)[C@@H]2C[C@@H](O)CN2C(=O)[C@@H](NC(=O)COCCN2CCN(CCOc3ccc([C@@H]4c5ccc(OCc6ccccc6)cc5CC[C@@H]4c4ccccc4)cc3)CC2)C(C)(C)C)cc1 ; Cc1ncsc1-c1ccc(CNC(=O)[C@@H]2C[C@@H](O)CN2C(=O)[C@@H](NC(=O)COCCN2CCN(CCOc3ccc([C@@H]4c5ccc(O)cc5CC[C@@H]4c4ccccc4)cc3)CC2)C(C)(C)C)cc1 ; "C1"]

2-(4-((1R,2S)-6-(Benzyloxy)-2-phenyl-1,2,3,4-tetrahydronaphthalen-1-yl)-phenoxy)acetaldehyde 24e. To a solution of compound 24d (263 mg, 0.518 mmol) in THE (8 mL) at −70° C. was added 1M LiAlH₄ in THE (1.5 mL) dropwise. After stirred at −70° C. for 30 min, the reaction was quenched with sat. aqueous NH₄Cl (10 mL) and the reaction mixture was extracted with EtOAc (20 mL×2). The combined organic layers were dried over anhydrous Na₂SO₄, filtered, and concentrated to give afford 24e (232 mg) as a crude product, which was used directly in the next step without further purification. MS (ESI) m/z: 466.2 [M+NH₄]⁺.

Ethyl 2-(2-(4-(2-(4-((1R,2S)-6-(benzyloxy)-2-phenyl-1,2,3,4-tetrahydro-naphthalen-1-yl)phenoxy)ethyl)piperazin-1-yl)ethoxy)acetate 24f. To a solution of compound 24e (232 mg, 0.517 mmol) and ethyl 2-(2-(piperazin-1-yl) ethoxy)acetate (224 mg, 1.03 mmol) in DCM (8 mL) and MeOH (2 mL) were added NaBH₃CN (163 mg, 2.59 mmol) and AcOH (1 drop). After stirred overnight, the reaction mixture was concentrated to yield a crude product, which was purified by silica gel column chromatography eluting with MeOH in DCM to afford compound 24f (160 mg) in 48% yield. MS (ESI) m/z: 649.3 [M+H]⁺.

2-(2-(4-(2-(4-((1R,2S)-6-(Benzyloxy)-2-phenyl-1,2,3,4-tetrahydronaphthalen-1-yl)phenoxy)ethyl)piperazin-1-yl)ethoxy)acetic acid 24g. To a solution of compound 24f (160 mg, 0.247 mmol) in THE (4 mL), MeOH (1 mL), and H₂O (1 mL) was added LiOH (21 mg, 0.493 mmol). After stirred overnight, the reaction mixture was concentrated, diluted with H₂O (3 mL), and neutralized to pH 3 with 1N HCl. The resulting precipitates were collected by filtration and dried to afford compound 24g (153 mg) as a crude product, which was used directly in the next step without further purification. MS (ESI) m/z: 621.3 [M+H]⁺.

(2S,4R)-1-((S)-2-(2-(2-(4-(2-(4-((1R,2S)-6-(Benzyloxy)-2-phenyl-1,2,3,4-tetrahydronaphthalen-1-yl)phenoxy)ethyl)piperazin-1-yl)ethoxy)acetamido)-3,3-dimethyl-butanoyl)-4-hydroxy-N-(4-(4-methylthiazol-5-yl)benzyl)pyrrolidine-2-carboxamide 24i. To a solution of compound 24g (87 mg, 0.14 mmol) and (2S,4R)-1-((S)-2-amino-3,3-dimethyl-butanoyl)-4-hydroxy-N-(4-(4-methylthiazol-5-yl)benzyl)pyrrolidine-2-carboxamide 24h (60 mg, 0.14 mmol) in DMF (8 mL) were added DIEA (54 mg, 0.42 mmol), HOBt (28 mg, 0.21 mmol), and EDCI (40 mg, 0.21 mmol). After stirred overnight, the reaction mixture was concentrated to yield a crude product, which was purified by prep-TLC (DCM/MeOH, 15/1) to afford compound 24i (90 mg) in 63% yield. MS (ESI) m/z: 1033.5 [M+H]⁺.

(2S,4R)-4-Hydroxy-1-((S)-2-(2-(2-(4-(2-(4-((1R,2S)-6-hydroxy-2-phenyl-1,2,3,4-tetrahydronaphthalen-1-yl)phenoxy)ethyl)piperazin-1-yl)ethoxy)acetamido)-3,3-dimethyl-butanoyl)-N-(4-(4-methylthiazol-5-yl)benzyl)pyrrolidine-2-carboxamide C1. To a solution of compound 24i (50 mg, 0.048 mmol) in THE (4 mL) and MeOH (4 mL) were added Pd/C (50 mg) and Pd(OH)₂/C (50 mg). After stirred under H₂ for 12 h, the reaction mixture was filtered and concentrated to yield a crude product, which was purified by reverse phase prep-HPLC to afford compound C1 (2.1 mg) in 4.6% yield. ¹H NMR (400 MHz, DMSO-d₆) δ 9.14 (s, 1H), 8.95 (s, 1H), 8.59 (t, J=6.0 Hz, 1H), 7.41-7.34 (m, 4H), 7.19-7.09 (m, 4H), 6.83 (d, J=6.8 Hz, 2H), 6.64-6.60 (m, 2H), 6.51-6.47 (m, 3H), 6.26 (d, J=8.4 Hz, 2H), 5.32 (t, J=4.8 Hz, 1H), 5.16 (s, 1H), 4.56 (d, J=9.6 Hz, 1H), 4.46-4.35 (m, 2H), 4.26-4.16 (m, 2H), 3.94-3.83 (m, 3H), 3.68-3.56 (m, 5H), 3.01-2.87 (m, 4H), 2.67 (d, J=1.6 Hz, 1H), 2.55-2.54 (m, 2H), 2.45-2.33 (m, 6H), 2.09-1.99 (m, 5H), 1.96-1.87 (m, 1H), 1.72-1.69 (m, 1H), 1.47-1.44 (m, 1H), 0.93 (s, 9H); MS (ESI) m/z: 943.2 [M+H]⁺.

### Example 25

### Preparation of 3-(4-(2-(4-(4-(4-((5-hydroxy-2-(4-hydroxyphenyl)-3-methyl-3H-indol-3-yl)-methyl)phenoxy)butyl)piperazin-1-yl)-2-oxoethoxy)-1-oxoisoindolin-2-yl)piperidine-2,6-dione D1

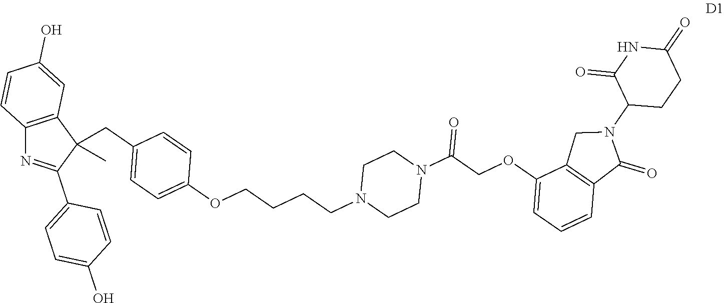 [SMILES: CC1(Cc2ccc(OCCCCN3CCN(C(=O)COc4cccc5c4CN(C4CCC(=O)NC4=O)C5=O)CC3)cc2)C(c2ccc(O)cc2)=Nc2ccc(O)cc21]

Compound D1 was prepared as shown in Schemes 25A and 25B.

5-(Benzyloxy)-2-(4-(benzyloxy)phenyl)-3-methyl-1H-indole 25a. To a solution of 1-(4-(benzyloxy)phenyl)propan-1-one (1.9 g, 8.0 mmol) and (4-(benzyloxy)phenyl)hydrazine (2.0 g, 8.0 mmol) in EtOH (20 mL) was added 5 drops of conc. HCl. After refluxed for 2 h, the reaction mixture was cooled to room temperature and the resulting precipitates were collected by filtration and washed with cold EtOH and PE to afford compound 25a (2.8 g) in 84% yield. ¹H NMR (400 MHz, DMSO-d₆) δ 10.87 (s, 1H), 7.57 (d, J=8.8 Hz, 2H), 7.49 (d, J=7.2 Hz, 4H), 7.43-7.30 (m, 6H), 7.21 (d, J=8.8 Hz, 1H), 7.14 (d, J=8.8 Hz, 2H), 7.08 (d, J=2.0 Hz, 1H), 6.79 (dd, J=2.4, 8.8 Hz, 1H), 5.17 (s, 2H), 5.12 (s, 2H), 2.33 (s, 3H).

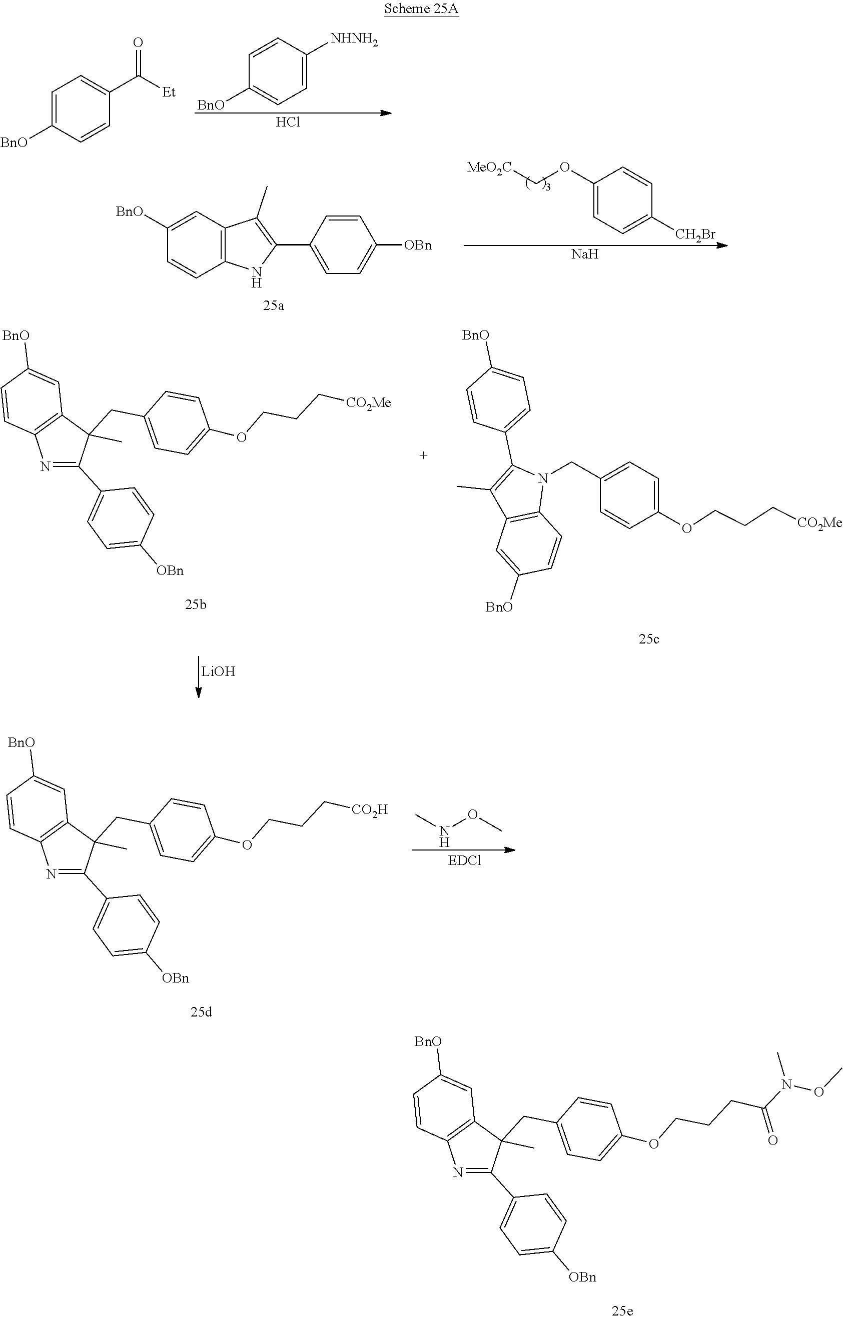

Methyl 4-(4-((5-(benzyloxy)-2-(4-(benzyloxy)phenyl)-3-methyl-3H-indol-3-yl)methyl)phenoxy)butanoatemethyl 25b and 4-(4-((5-(benzyloxy)-2-(4-(benzyloxy)phenyl)-3-methyl-1H-indol-1-yl)methyl)phenoxy)butanoate 25c. To a solution of compound 25a (5.93 g, 14.15 mmol) in DMF (40 mL) at 0° C. was added NaH (849 mg, 21.23 mmol). After the mixture was stirred at 0° C. for 10 min, methyl 4-(4-(bromomethyl)phenoxy)butanoate (4.86 g, 16.98 mmol) in DMF (5 mL) was added. The reaction mixture was stirred at 0° C. for 20 min, and then diluted with H₂O (10 mL) and extracted with EtOAc (40 mL×2). The combined organic layers were washed with H₂O (30 mL), dried over anhydrous Na₂SO₄, filtered, and concentrated to yield a crude product, which was purified by silica gel column chromatography eluting with EtOAc in PE to afford compound 25b (2.6 g) in 30% yield and compound 25c (3.9 g) in 44% yield. Compound 25b: ¹H NMR (400 MHz, DMSO-d₆) δ 7.48-7.47 (m, 4H), 7.43-7.29 (m, 8H), 7.20 (d, J=8.8 Hz, 1H), 7.13-7.11 (m, 3H), 6.81 (dd, J=2.4, 8.8 Hz, 1H), 6.76-6.71 (m, 4H), 5.15-5.12 (m, 6H), 3.87 (t, J=6.4 Hz, 2H), 2.39 (t, J=7.6 Hz, 2H), 2.16 (s, 3H), 1.93-1.86 (m, 2H); MS (ESI) m/z: 655.3 [M+H]⁺.

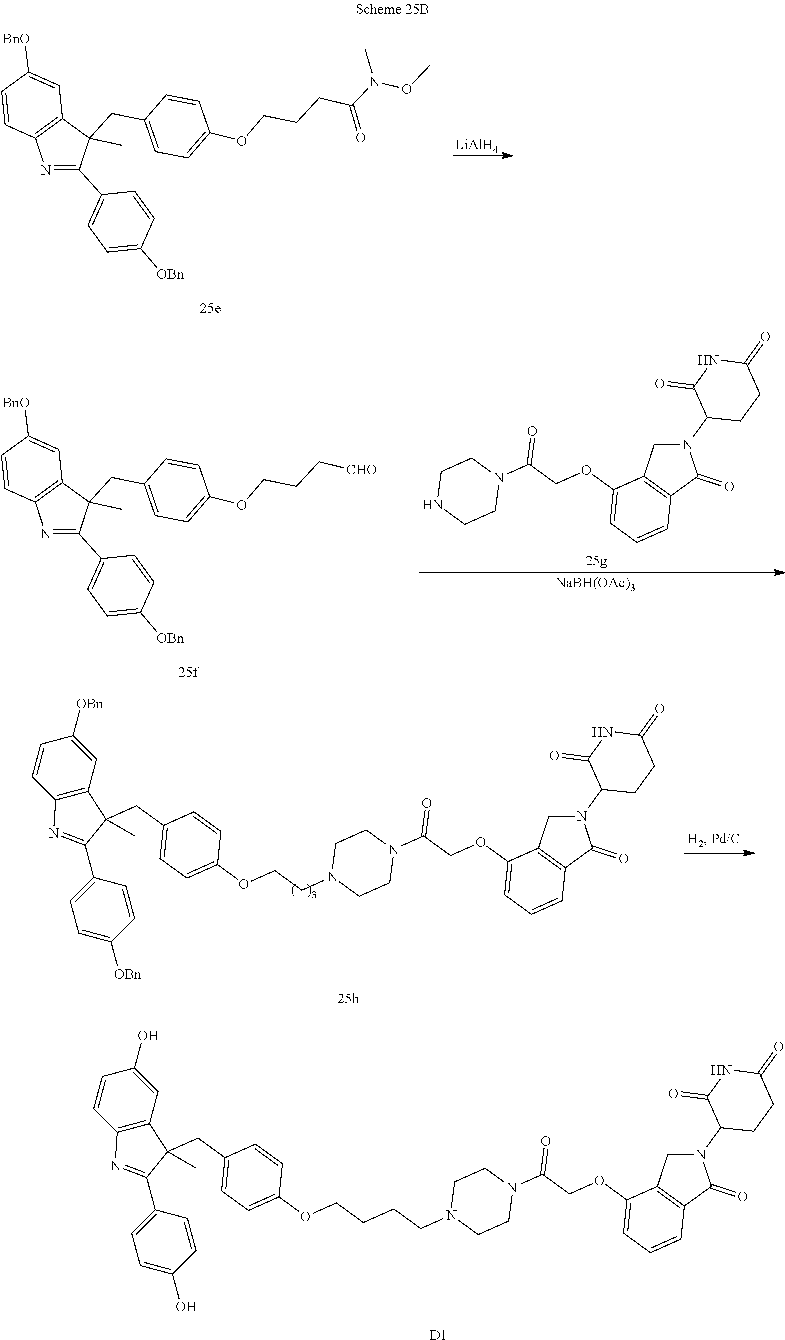

4-(4-((5-(Benzyloxy)-2-(4-(benzyloxy)phenyl)-3-methyl-3H-indol-3-yl)methyl)-phenoxy)butanoic acid 25d. To a solution of compound 25b (2.495 g, 3.99 mmol) in THE (20 mL), MeOH (5 mL), and H₂O (5 mL) at 0° C. was added LiOH—H₂O (343 mg, 7.98 mmol). After stirred at room temperature overnight and then cooled to 0° C., the reaction mixture was neutralized to pH 5 with 2N HCl and extracted with EtOAc (40 mL×2). The combined organic layers were washed with H₂O (20 mL), dried over anhydrous Na₂SO₄, filtered, and concentrated to afford compound 25d (2.43 g) as a crude product, which was used directly in the next step without further purification. MS (ESI) m/z: 612.3 [M+H]⁺.

4-(4-((5-(Benzyloxy)-2-(4-(benzyloxy)phenyl)-3-methyl-3H-indol-3-yl)methyl)-phenoxy)-N-methoxy-N-methylbutanamide 25e. To a solution of compound 25d (2.43 g, 3.98 mmol) in DCM (40 mL) at 0° C. were added N,O-dimethylhydroxylamine hydrochloride (536 mg, 5.97 mmol), EDCI·HCl (1.14 g, 5.97 mmol), TEA (1.2 g, 11.94 mmol), and DMAP (97 mg, 0.796 mmol). After stirred overnight, the reaction mixture was diluted with H₂O (10 mL) and extracted with EtOAc (40 mL×2). The combined organic layers were washed with H₂O (20 mL), dried over anhydrous Na₂SO₄, filtered, and concentrated to yield a crude product, which was purified by silica gel column chromatography eluting with EtOAc in PE to afford compound 25e (1.84 g) in 71% yield. ¹H NMR (400 MHz, DMSO-d₆) δ 8.20 (d, J=11.2 Hz, 2H), 7.55-7.53 (m, 4H), 7.48-7.37 (m, 2H), 7.29-7.27 (m, 2H), 7.19 (d, J=11.6 Hz, 2H), 6.92 (dd, J=2.4, 11.2 Hz, 1H), 6.44-6.36 (m, 4H), 5.25 (s, 2H), 5.21 (s, 2H), 3.79 (t, J=8.4 Hz, 2H), 3.62 (s, 3H), 3.51-3.33 (m, 4H), 3.09 (s, 3H), 2.48 (t, J=9.6 Hz, 2H), 1.86-1.82 (m, 2H), 1.62 (s, 3H); MS (ESI) m/z: 655.3 [M+H]⁺.

4-(4-((5-(Benzyloxy)-2-(4-(benzyloxy)phenyl)-3-methyl-3H-indol-3-yl)methyl)-phenoxy)butanal 25f. To a solution of compound 25e (120 mg, 0.18 mmol) in THE (4 mL) at −70° C. under N₂ was added 1M LiAlH₄ in THF (0.54 mL) dropwise. After stirred at −70° C. for 1 h, the reaction was quenched with sat. aqueous NH₄Cl (5 mL) and the reaction mixture was extracted with EtOAc (10 mL×2). The combined organic layers were dried over anhydrous Na₂SO₄, filtered, and concentrated to afford compound 25f (107 mg) as a crude product, which was used directly in the next step without further purification. MS (ESI) m/z: 596.2 [M+H]⁺.

3-(4-(2-(4-(4-(4-((5-(Benzyloxy)-2-(4-(benzyloxy)phenyl)-3-methyl-3H-indol-3-yl)methyl)phenoxy)butyl)piperazin-1-yl)-2-oxoethoxy)-1-oxoisoindolin-2-yl)piperidine-2,6-dione 25h. To a solution of compound 25f (0.18 mmol) in DCM (4 mL) and MeOH (1 mL) were added 3-(1-oxo-4-(2-oxo-2-(piperazin-1-yl)ethoxy)isoindolin-2-yl)piperidine-2,6-dione 25g (89 mg, 0.18 mmol) and NaBH(OAc)₃ (76 mg, 0.36 mmol), followed by addition of AcOH (1 drop). After stirred overnight, the reaction mixture was concentrated to yield a crude product, which was purified by silica gel column chromatography eluting with MeOH in DCM to afford compound 25h (90 mg) in 52% yield. MS (ESI) m/z: 966.4 [M+H]⁺.

3-(4-(2-(4-(4-(4-((5-Hydroxy-2-(4-hydroxyphenyl)-3-methyl-3H-indol-3-yl)-methyl)phenoxy)butyl)piperazin-1-yl)-2-oxoethoxy)-1-oxoisoindolin-2-yl)piperidine-2,6-dione D1. To a solution of compound 25h (90 mg, 0.093 mmol) in THE (5 mL) was added Pd/C (50 mg). After stirred under H₂ overnight, the reaction mixture was filtered and concentrated to yield a crude product, which was purified by reverse phase prep-HPLC to afford compound D1 (34 mg) in 47% yield. ¹H NMR (400 MHz, DMSO-d₆) δ 10.98 (s, 1H), 8.16 (s, 1H), 8.03 (d, J=9.2 Hz, 2H), 7.45 (t, J=8.0 Hz, 1H), 7.32 (d, J=7.2 Hz, 1H), 7.16-7.10 (m, 2H), 6.9-6.84 (m, 3H), 6.63 (dd, J=2.0, 8.0 Hz, 1H), 6.50-6.36 (m, 4H), 5.10 (dd, J=5.2, 13.2 Hz, 1H), 4.99 (s, 2H), 4.41-4.24 (m, 2H), 3.77 (t, J=6.0 Hz, 2H), 3.28-3.25 (m, 4H), 2.94-2.86 (m, 1H), 2.61-2.51 (m, 3H), 2.49-2.43 (m, 1H), 2.40-2.22 (m, 5H), 2.01-1.99 (m, 1H), 1.62-1.57 (m, 2H), 1.54 (s, 3H), 1.52-1.46 (m, 2H); MS (ESI) m/z: 786.5 [M+H]⁺.

### Example 26

### Preparation of 3-(7-(2-(4-(4-(4-((5-hydroxy-2-(4-hydroxyphenyl)-3-methyl-3H-indol-3-yl)-methyl)phenoxy)butyl)piperazin-1-yl)-2-oxoethoxy)-1-methyl-1H-indazol-3-yl)piperidine-2,6-dione D4

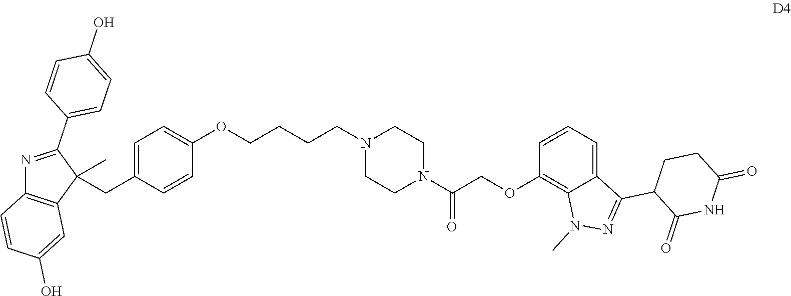 [SMILES: Cn1nc(C2CCC(=O)NC2=O)c2cccc(OCC(=O)N3CCN(CCCCOc4ccc(CC5(C)C(c6ccc(O)cc6)=Nc6ccc(O)cc65)cc4)CC3)c21]

Compound D4 was prepared as shown in Scheme 26.

3-(7-(2-(4-(4-(4-((5-(Benzyloxy)-2-(4-(benzyloxy)phenyl)-3-methyl-3H-indol-3-yl)methyl)phenoxy)butyl)piperazin-1-yl)-2-oxoethoxy)-1-methyl-1H-indazol-3-yl)piperidine-2,6-dione 26a. To a solution of 4-(4-((5-(benzyloxy)-2-(4-(benzyloxy)phenyl)-3-methyl-3H-indol-3-yl)methyl)phenoxy)butanal 25f (0.229 mmol) in DCM (8 mL) and MeOH (2 mL) were added 3-(1-methyl-7-(2-oxo-2-(piperazin-1-yl)ethoxy)-1H-indazol-3-yl)piperidine-2,6-dione 6h (0.229 mmol) and NaBH(OAc)₃ (97 mg, 0.458 mmol). After stirred for 8 h, the reaction mixture was concentrated to yield a crude product, which was purified by silica gel column chromatography eluting with MeOH in DCM to afford compound 26a (140 mg) in 63% yield. MS (ESI) m/z: 965.1 [M+H]⁺.

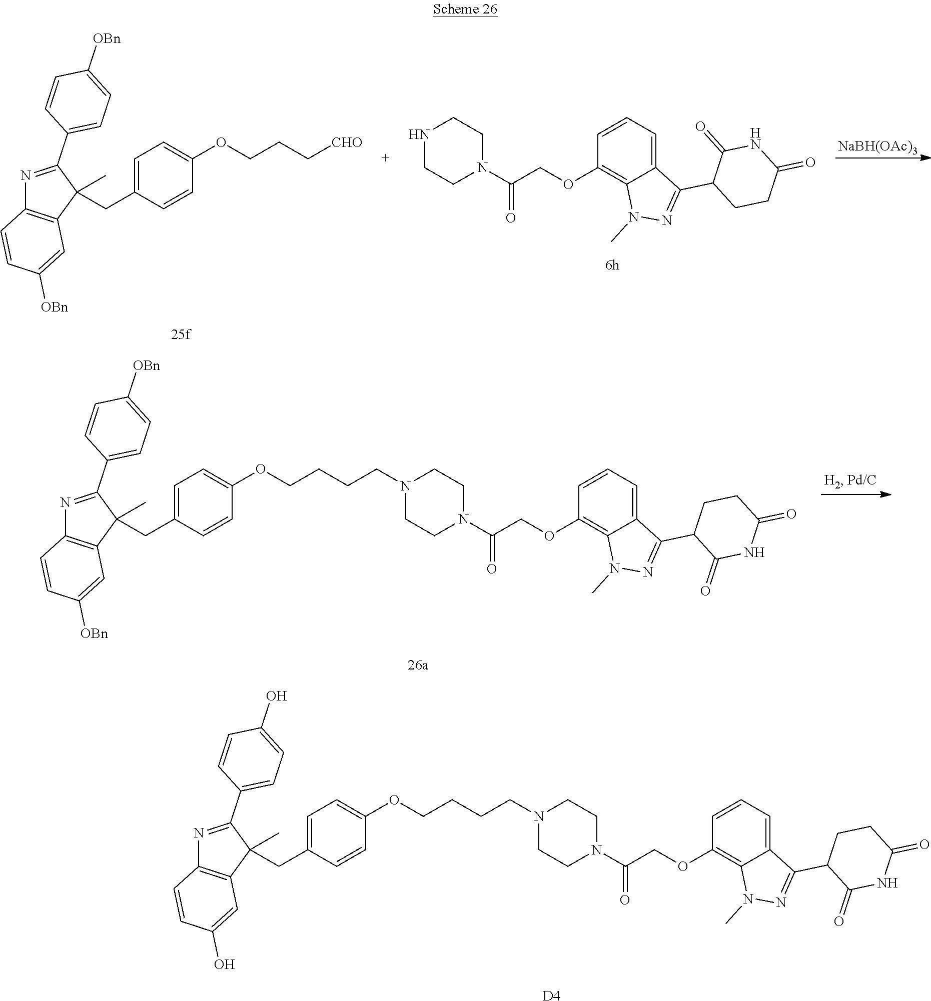 [SMILES: CC1(Cc2ccc(OCCCC=O)cc2)C(c2ccc(OCc3ccccc3)cc2)=Nc2ccc(OCc3ccccc3)cc21 ; Cn1nc(C2CCC(=O)NC2=O)c2cccc(OCC(=O)N3CCNCC3)c21 ; Cn1nc(C2CCC(=O)NC2=O)c2cccc(OCC(=O)N3CCN(CCCCOc4ccc(CC5(C)C(c6ccc(OCc7ccccc7)cc6)=Nc6ccc(OCc7ccccc7)cc65)cc4)CC3)c21 ; "NaBH(OAc)3" ; Cn1nc(C2CCC(=O)NC2=O)c2cccc(OCC(=O)N3CCN(CCCCOc4ccc(CC5(C)C(c6ccc(O)cc6)=Nc6ccc(O)cc65)cc4)CC3)c21 ; "D4"]

3-(7-(2-(4-(4-(4-((5-Hydroxy-2-(4-hydroxyphenyl)-3-methyl-3H-indol-3-yl)-methyl)phenoxy)butyl)piperazin-1-yl)-2-oxoethoxy)-1-methyl-1H-indazol-3-yl)piperidine-2,6-dione D4. To a solution of compound 26a (140 mg, 0.145 mmol) in THE (10 mL) was added Pd/C (40 mg). After stirred under H₂ for 8 h, the reaction mixture was filtered and concentrated to yield a crude product, which was purified by silica gel column chromatography eluting with MeOH in DCM to afford compound D4 (32.7 mg) in 29% yield. ¹H NMR (400 MHz, DMSO-d₆) δ 10.87 (s, 1H), 9.95 (s, 1H), 9.32 (s, 1H), 8.13 (s, 1H), 8.03 (d, J=8.8 Hz, 2H), 7.24 (d, J=8.4 Hz, 1H), 7.12 (d, J=8.0 Hz, 1H), 6.96 (t, J=7.6 Hz, 1H), 6.90-6.87 (m, 3H), 6.83 (d, J=7.6 Hz, 1H), 6.63 (dd, J=2.0, 8.0 Hz, 1H), 6.64-6.41 (m, 4H), 5.00 (s, 2H), 4.33 (dd, J=4.8, 9.6 Hz, 1H), 4.23 (s, 3H), 3.77 (t, J=6.0 Hz, 2H), 3.47-3.40 (m, 6H), 3.28-3.25 (m, 2H), 2.67-2.57 (m, 3H), 2.39-2.31 (m, 4H), 2.20-2.14 (m, 1H), 1.63-1.59 (m, 2H), 1.54-1.49 (m, 5H); MS (ESI) m/z: 785.5 [M+H]⁺.

### Example 27

### Preparation of 3-(4-(2-(4-(4-(4-((5-hydroxy-2-(4-hydroxyphenyl)-3-methyl-1H-indol-1-yl)-methyl)phenoxy)butyl)piperazin-1-yl)-2-oxoethoxy)-1-oxoisoindolin-2-yl)piperidine-2,6-dione E1

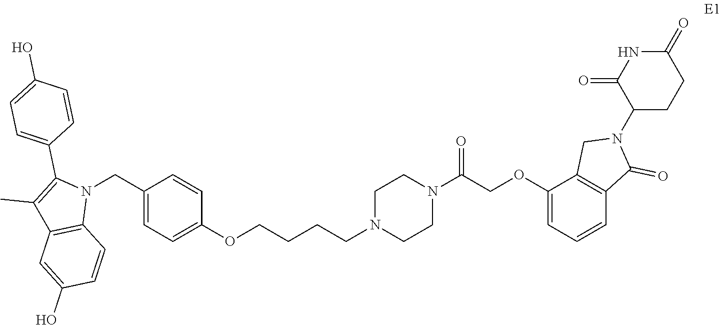 [SMILES: Cc1c(-c2ccc(O)cc2)n(Cc2ccc(OCCCCN3CCN(C(=O)COc4cccc5c4CN(C4CCC(=O)NC4=O)C5=O)CC3)cc2)c2ccc(O)cc12]

Compound E1 was prepared as shown in Scheme 27.

4-(4-((5-(Benzyloxy)-2-(4-(benzyloxy)phenyl)-3-methyl-1H-indol-1-yl)methyl)-phenoxy)butanal 27b. To a solution of 4-(4-((5-(benzyloxy)-2-(4-(benzyloxy)phenyl)-3-methyl-1H-indol-1-yl)methyl)phenoxy)-N-methoxy-N-methylbutanamide 27a (150 mg, 0.23 mmol) in THE (3 mL) at −70° C. under N₂ was added 1M LiAlH₄ in THF (0.69 mL) dropwise. After stirred at −70° C. for 1 h, the reaction was quenched with sat. aqueous NH₄Cl (5 mL) and the reaction mixture was extracted with EtOAc (10 mL×2). The combined organic layers were dried over anhydrous Na₂SO₄, filtered, and concentrated to afford compound 27b (137 mg) as a crude product, which was used directly in the next step without further purification. MS (ESI) m/z: 596.2 [M+H]⁺.

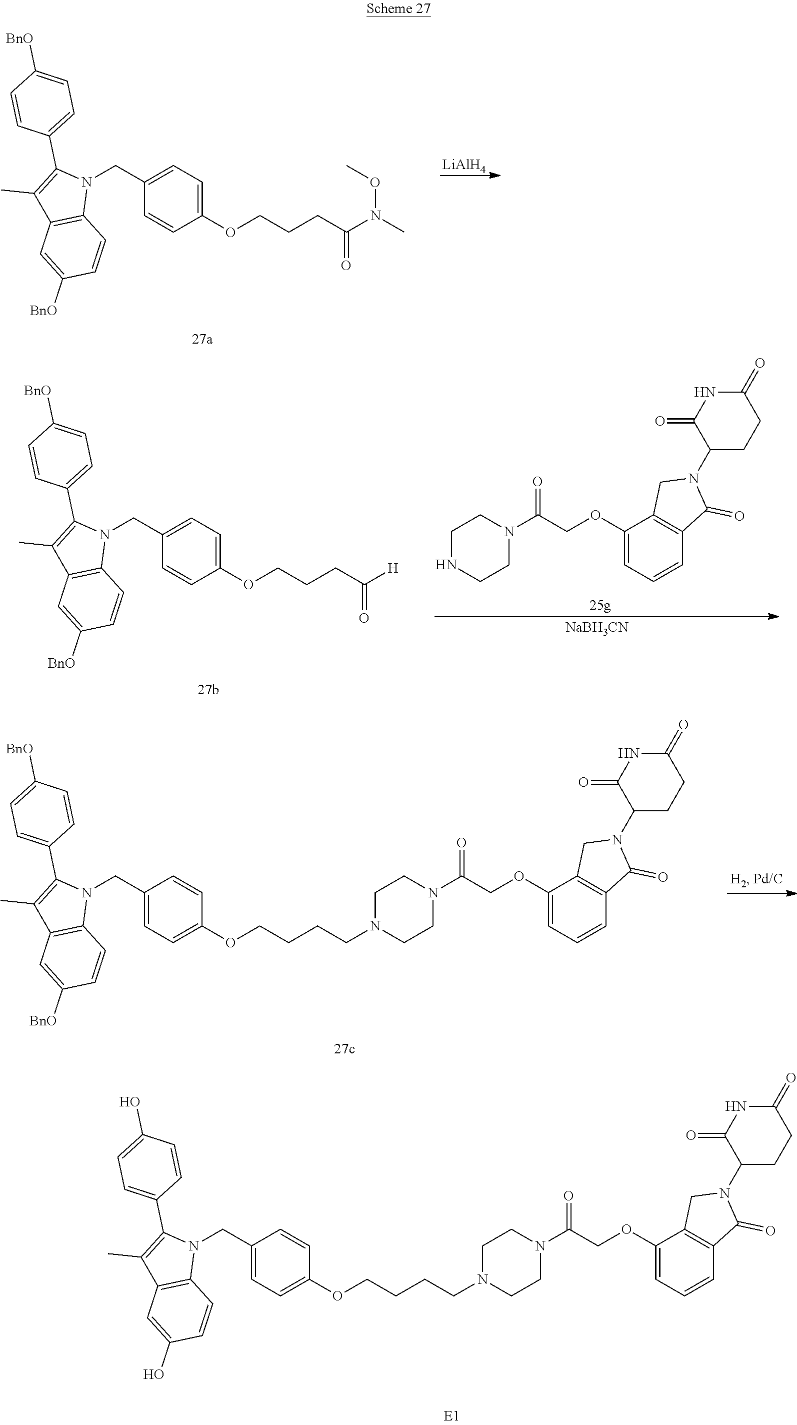

3-(4-(2-(4-(4-(4-((5-(Benzyloxy)-2-(4-(benzyloxy)phenyl)-3-methyl-1H-indol-1-yl)methyl)phenoxy)butyl)piperazin-1-yl)-2-oxoethoxy)-1-oxoisoindolin-2-yl)piperidine-2,6-dione 27c. To a solution of compound 27b (137 mg, 0.23 mmol) in DCM (4 mL) and MeOH (1 mL) were added 3-(1-oxo-4-(2-oxo-2-(piperazin-1-yl)ethoxy)isoindolin-2-yl)piperidine-2,6-dione 25g (89 mg, 0.23 mmol) and NaBH₃CN (43 mg, 0.69 mmol), followed by addition of AcOH (1 drop). After stirred overnight, the reaction mixture was concentrated to yield a crude product, which was purified by silica gel column chromatography eluting with MeOH in DCM to afford compound 27c (166 mg) in 65% yield. MS (ESI) m/z: 966.4 [M+H]⁺.

3-(4-(2-(4-(4-(4-((5-Hydroxy-2-(4-hydroxyphenyl)-3-methyl-1H-indol-1-yl)-methyl)phenoxy)butyl)piperazin-1-yl)-2-oxoethoxy)-1-oxoisoindolin-2-yl)piperidine-2,6-dione E1. To a solution of compound 27c (166 mg, 0.17 mmol) in THE (5 mL) was added Pd/C (100 mg). After stirred under H₂ overnight, the reaction mixture was filtered and concentrated to yield a crude product, which was purified by reverse phase prep-HPLC to afford compound E1 (6.9 mg) in 5% yield. ¹H NMR (400 MHz, DMSO-d₆) δ 10.98 (s, 1H), 9.66 (s, 1H), 8.68 (s, 1H), 7.45 (t, J=7.6 Hz, 1H), 7.32 (d, J=7.6 Hz, 1H), 7.17-7.12 (m, 3H), 7.06 (d, J=8.8 Hz, 1H), 6.85 (d, J=8.8 Hz, 2H), 6.80 (d, J=2.4 Hz, 1H), 6.77-6.72 (m, 4H), 6.57 (dd, J=2.0, 8.8 Hz, 1H), 5.13-5.08 (m, 3H), 5.00 (s, 2H), 4.41-4.24 (m, 2H), 3.88 (t, J=6.0 Hz, 2H), 3.48-3.39 (m, 4H), 2.67-2.56 (m, 2H), 2.44-2.28 (m, 7H), 2.10 (s, 3H), 2.01-1.98 (m, 1H), 1.69-1.64 (m, 2H), 1.55-1.52 (m, 2H); MS (ESI) m/z: 786.5 [M+H]⁺.

### Example 28

### Preparation of 3-(5-(4-((1-(4-((5-hydroxy-2-(4-hydroxyphenyl)-3-methyl-1H-indol-1-yl)-methyl)phenyl)piperidin-4-yl)methyl)piperazin-1-yl)-1-oxoisoindolin-2-yl)piperidine-2,6-dione E4

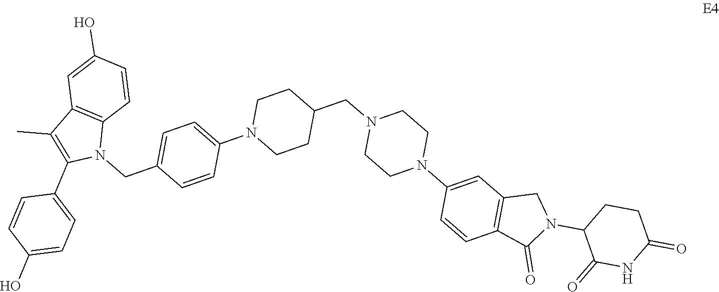 [SMILES: Cc1c(-c2ccc(O)cc2)n(Cc2ccc(N3CCC(CN4CCN(c5ccc6c(c5)CN(C5CCC(=O)NC5=O)C6=O)CC4)CC3)cc2)c2ccc(O)cc12]

Compound E4 was prepared as shown in Scheme 28.

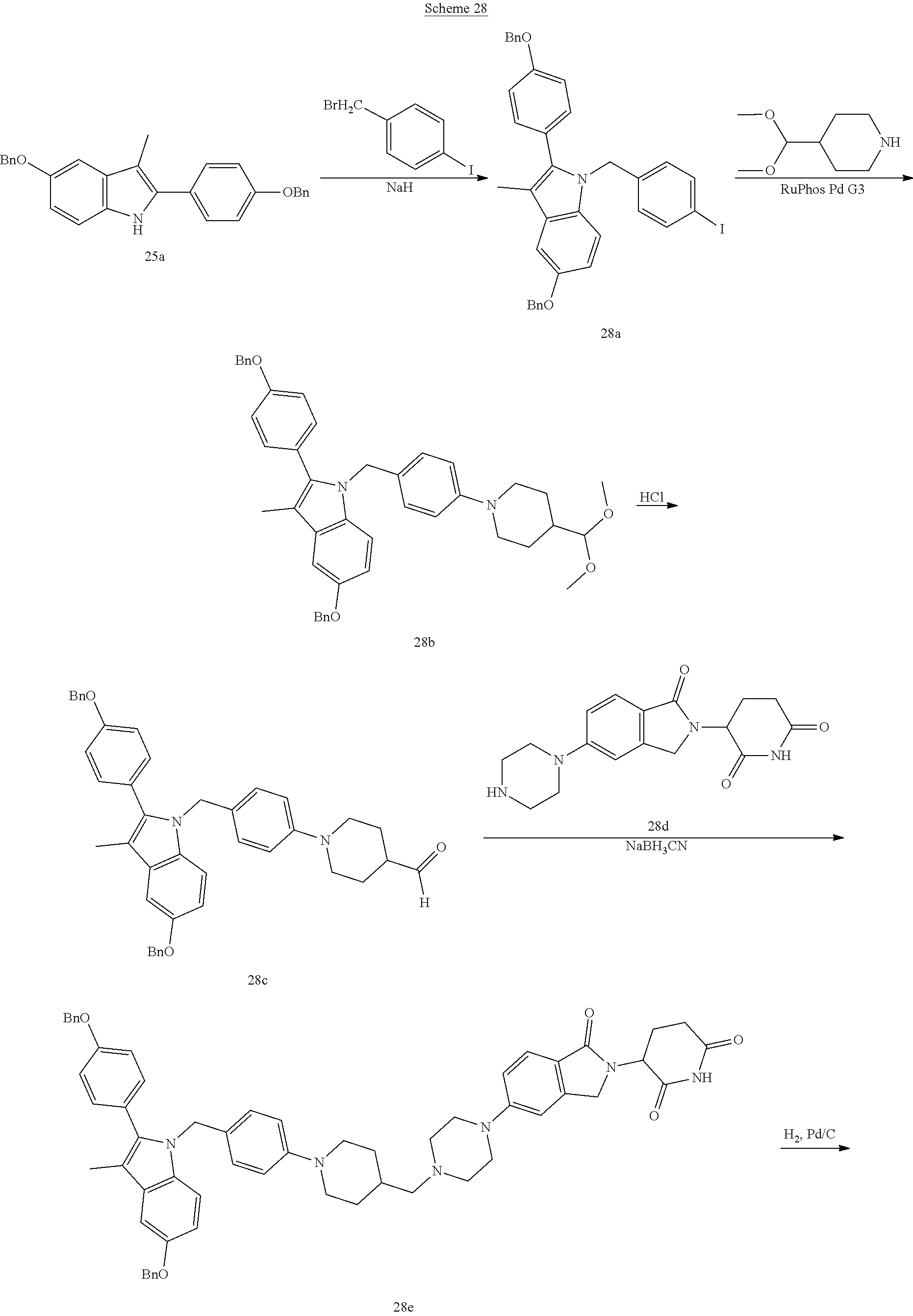

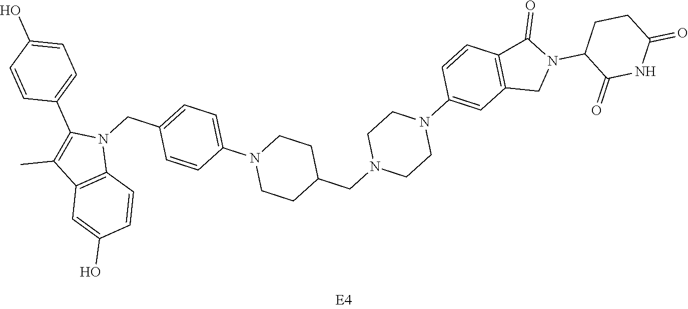 [SMILES: Cc1c(-c2ccc(O)cc2)n(Cc2ccc(N3CCC(CN4CCN(c5ccc6c(c5)CN(C5CCC(=O)NC5=O)C6=O)CC4)CC3)cc2)c2ccc(O)cc12]

5-(Benzyloxy)-2-(4-(benzyloxy)phenyl)-1-(4-iodobenzyl)-3-methyl-1H-indole 28a. To a solution of 5-(benzyloxy)-2-(4-(benzyloxy)phenyl)-3-methyl-1H-indole 25a (1 g, 2.39 mmol) in DMF (10 mL) at 0° C. was added NaH (144 mg, 3.59 mmol). After the mixture was stirred at 0° C. for 10 min, 1-(bromomethyl)-4-iodobenzene (852 mg, 2.87 mmol) in DMF (1 mL) was added. The reaction mixture was stirred at 0° C. for 10 min, and then diluted with H₂O (10 mL) and extracted with EtOAc (20 mL×2). The combined organic layers were washed with H₂O (10 mL), dried over anhydrous Na₂SO₄, filtered, and concentrated to yield a crude product, which was purified by silica gel column chromatography eluting with EtOAc in PE to afford compound 28a (556 mg) in 37% yield. ¹H NMR (400 MHz, DMSO-d₆) δ 7.53 (d, J=8.4 Hz, 2H), 7.49 (d, J=7.2 Hz, 2H), 7.46-7.34 (m, 8H), 7.19 (d, J=8.4 Hz, 2H), 7.14 (d, J=2.4 Hz, 1H), 7.02-6.99 (m, 3H), 6.88 (dd, J=2.4, 8.8 Hz, 1H), 6.67 (d, J=8.4 Hz, 2H), 5.13 (s, 2H), 5.10-5.09 (m, 4H), 2.24 (s, 3H); MS (ESI) m/z: 636.1 [M+H]⁺.

5-(Benzyloxy)-2-(4-(benzyloxy)phenyl)-1-(4-(4-(dimethoxymethyl)piperidin-1-yl)benzyl)-3-methyl-1H-indole 28b. To a solution of compound 28a (300 mg, 0.472 mmol) in dioxane (15 mL) were added 4-(dimethoxymethyl)piperidine (232 mg, 1.42 mmol), Cs₂CO₃ (306 mg, 0.944 mmol), RuPhos Pd G3 (79 mg, 0.0944 mmol), and RuPhos (88 mg, 0.19 mmol). After heated at 100° C. overnight under N₂, the reaction mixture was filtered and concentrated to yield a crude product, which was purified by silica gel column chromatography eluting with EtOAc in PE to afford 28b (250 mg) in 80% yield. MS (ESI) m/z: 667.4 [M+H]⁺.

1-(4-((5-(Benzyloxy)-2-(4-(benzyloxy)phenyl)-3-methyl-1H-indol-1-yl)methyl)-phenyl)piperidine-4-carbaldehyde 28c. To a solution of compound 28b (250 mg, 0.375 mmol) in acetone (10 mL) was added 2N HCl (0.75 mmol, 0.38 mL). After stirred at 70° C. for 30 min, the reaction mixture was concentrated, diluted with H₂O (10 mL), and extracted with EtOAc (20 mL×2). The combined organic layers were dried over anhydrous Na₂SO₄, filtered, and concentrated to afford compound 28c (209 mg) in 81% yield. MS (ESI) m/z: 621.3 [M+H]⁺.

3-(5-(4-((1-(4-((5-(Benzyloxy)-2-(4-(benzyloxy)phenyl)-3-methyl-1H-indol-1-yl)methyl)phenyl)piperidin-4-yl)methyl)piperazin-1-yl)-1-oxoisoindolin-2-yl)piperidine-2,6-dione 28e. To a solution of 3-(1-oxo-5-(piperazin-1-yl)isoindolin-2-yl)piperidine-2,6-dione 28d (144 mg, 0.337 mmol) in DCM (16 mL) and MeOH (4 mL) at 0° C. were added compound 28c (209 mg, 0.337 mmol) and NaBH₃CN (64 mg, 1.011 mmol). After stirred overnight, the reaction mixture was concentrated to yield a crude product, which was purified by silica gel column chromatography eluting with MeOH in DCM to afford compound 28e (250 mg) in 80% yield. MS (ESI) m/z: 933.5 [M+H]⁺.

3-(5-(4-((1-(4-((5-Hydroxy-2-(4-hydroxyphenyl)-3-methyl-1H-indol-1-yl)-methyl)phenyl)piperidin-4-yl)methyl)piperazin-1-yl)-1-oxoisoindolin-2-yl)piperidine-2,6-dione E4. To a solution of compound 28e (250 mg, 0.267 mmol) in THE (30 mL) was added Pd/C (200 mg). After stirred for 48 h under H₂, the reaction mixture was filtered and concentrated to yield a crude product, which was purified by reverse phase prep-HPLC to afford compound E4 (63 mg) in 32% yield. ¹H NMR (400 MHz, DMSO-d₆) δ 10.95 (s, 1H), 9.70 (s, 1H), 8.66 (s, 1H), 8.15 (s, 1H), 7.51 (d, J=8.8 Hz, 1H), 7.17 (d, J=8.4 Hz, 2H), 7.08-7.04 (m, 3H), 6.86 (d, J=8.4 Hz, 2H), 6.79 (d, J=2.0 Hz, 1H), 6.75-6.68 (m, 4H), 6.56 (dd, J=2.4, 8.4 Hz, 1H), 5.09-5.02 (m, 3H), 4.34-4.17 (m, 2H), 3.57 (d, J=12.4 Hz, 2H), 3.32-3.27 (m, 4H), 2.93-2.87 (m, 1H), 2.59-2.50 (m, 7H), 2.38-2.34 (m, 1H), 2.19 (d, J=6.8 Hz, 2H), 2.10 (s, 3H), 1.97-1.94 (m, 1H), 1.76 (d J=12.0 Hz, 2H), 1.66-1.63 (m, 1H), 1.19-1.11 (m, 2H); MS (ESI) m/z: 776.1 [M+Na]⁺.

### Example 29

### Preparation of 3-(1-((4-(((4-(4-((5-hydroxy-2-(4-hydroxyphenyl)-3-methyl-1H-indol-1-yl)-methyl)phenoxy)butyl)amino)methyl)phenoxy)methyl)-4-oxo-4H-thieno[3,4-c]pyrrol-5(6H)-yl)-piperidine-2,6-dione E5

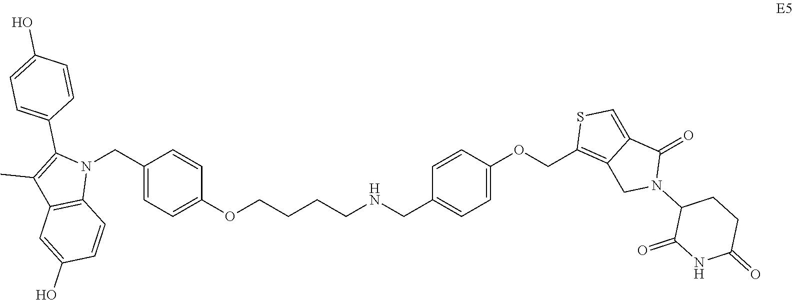 [SMILES: Cc1c(-c2ccc(O)cc2)n(Cc2ccc(OCCCCNCc3ccc(OCc4scc5c4CN(C4CCC(=O)NC4=O)C5=O)cc3)cc2)c2ccc(O)cc12]

Compound E5 was prepared as shown in Scheme 29.

4-(4-((5-Hydroxy-2-(4-hydroxyphenyl)-3-methyl-1H-indol-1-yl)methyl)-phenoxy)-N-methoxy-N-methylbutanamide 29a. To a solution of 4-(4-((5-(benzyloxy)-2-(4-(benzyloxy)phenyl)-3-methyl-1H-indol-1-yl)methyl)phenoxy)-N-methoxy-N-methylbutanamide 27a (150 mg, 0.229 mmol) in THE (10 mL) and MeOH (10 mL) was added Pd/C (45 mg) and Pd(OH)₂/C (45 mg) under N₂. After stirred under H₂ overnight, the reaction mixture was filtered and concentrated to yield a crude product, which was purified by silica gel column chromatography eluting with MeOH in DCM to afford compound 29a (70 mg) in 64% yield. MS (ESI) m/z: 475.2 [M+H]⁺.

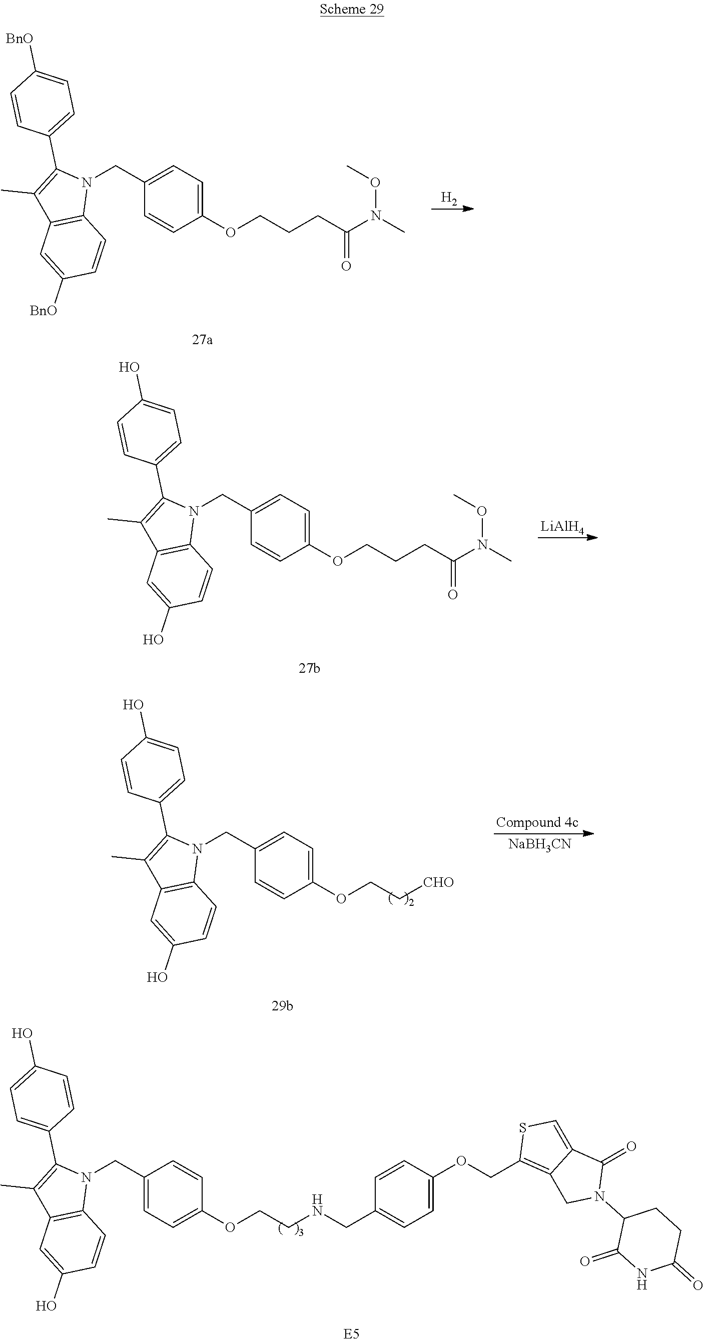

4-(4-((5-Hydroxy-2-(4-hydroxyphenyl)-3-methyl-1H-indol-1-yl)methyl)-phenoxy)butanal 29b. To a solution of compound 29a (70 mg, 0.148 mmol) in THE (10 mL) was added 1M LiAlH₄ in THE (0.37 mL, 0.369 mmol) dropwise at −70° C. under N₂. After stirred at −70° C. for 1 h, the reaction was quenched with sat. aqueous NH₄Cl (10 mL) and the reaction mixture was extracted with EtOAc (15 mL×3). The combined organic layers were washed with brine, dried over anhydrous Na₂SO₄, filtered, and concentrated to afford compound 29b (61 mg) as a crude product, which was used directly in the next step without further purification. MS (ESI) m/z: 416.2 [M+H]⁺.

3-(1-((4-(((4-(4-((5-Hydroxy-2-(4-hydroxyphenyl)-3-methyl-1H-indol-1-yl)-methyl)phenoxy)butyl)amino)methyl)phenoxy)methyl)-4-oxo-4H-thieno[3,4-c]pyrrol-5(6H)-yl)-piperidine-2,6-dione E5. To a solution of 3-(1-((4-(aminomethyl)phenoxy)methyl)-4-oxo-4H-thieno[3,4-c]pyrrol-5(6H)-yl)piperidine-2,6-dione 4c (57 mg) in DCM/MeOH (4/1, 2 mL) was added DIEA (2 drops) to adjust pH to 7, followed by addition of compound 29b (61 mg, crude) in DCM/MeOH (4/1, 2 mL) and AcOH (1 drop). After the mixture was stirred for 15 min, NaBH₃CN (94 mg, 0.444 mmol) was added. The reaction mixture was stirred overnight and then concentrated to yield a crude product, which was purified by silica gel column chromatography eluting with MeOH in DCM and further purified by reverse phase prep-HPLC to afford compound E5 (21.8 mg) in 19% yield. ¹H NMR (400 MHz, DMSO-d₆) δ 10.99 (s, 1H), 9.69 (s, 1H), 8.84 (brs, 2H), 8.70 (s, 1H), 8.03 (s, 1H), 7.43 (d, J=7.2 Hz, 2H), 7.16 (d, J=8.4 Hz, 2H), 7.07 (t, J=8.0 Hz, 3H), 6.86 (d, J=8.0 Hz, 2H), 6.80 (d, J=2.0 Hz, 1H), 6.78-6.71 (m, 4H), 6.57 (dd, J=1.6, 8.8 Hz, 1H), 5.34 (s, 2H), 5.11 (s, 2H), 5.03 (dd, J=4.8, 13.2 Hz, 1H), 4.38-4.20 (m, 2H), 4.08-4.00 (m, 2H), 3.87 (t, J=5.2 Hz, 2H), 2.96-2.81 (m, 3H), 2.64-2.53 (m, 1H), 2.40-2.25 (m, 1H), 2.10 (s, 3H), 2.02-1.93 (m, 1H), 1.78-1.63 (m, 4H); MS (ESI) m/z: 785.3 [M+H]⁺.

### Example 30

### Preparation of 3-(7-(2-(4-(4-(4-((2-(4-fluorophenyl)-5-hydroxy-3-methyl-1H-indol-1-yl)-methyl)phenoxy)butyl)piperazin-1-yl)-2-oxoethoxy)-1-methyl-1H-indazol-3-yl)piperidine-2,6-dione E9

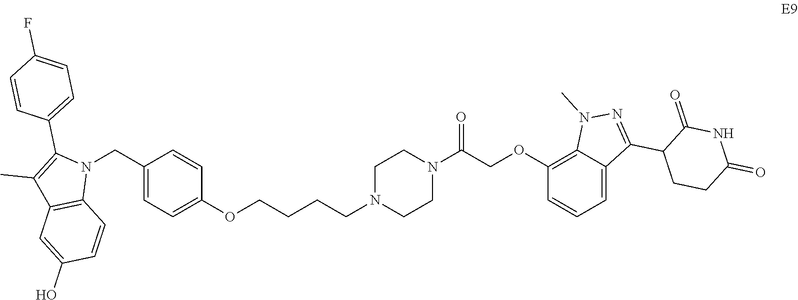 [SMILES: Cc1c(-c2ccc(F)cc2)n(Cc2ccc(OCCCCN3CCN(C(=O)COc4cccc5c(C6CCC(=O)NC6=O)nn(C)c45)CC3)cc2)c2ccc(O)cc12]

Compound E9 was prepared as shown in Scheme 30.

5-(Benzyloxy)-2-(4-fluorophenyl)-3-methyl-1H-indole 30a. To a solution of 1-(4-fluorophenyl)propan-1-one (6.4 g, 42 mmol) and (4-(benzyloxy)phenyl)hydrazine (9 g, 42 mmol) in EtOH (80 mL) was added 10 drops of conc. HCl. After refluxed for 2 h, the reaction mixture was cooled to room temperature. The resulting precipitates were collected by filtration and washed with cold EtOH and then petroleum ether to afford compound 30a (2 g) in 14% yield. MS (ESI) m/z: 332.1 [M+H]⁺.

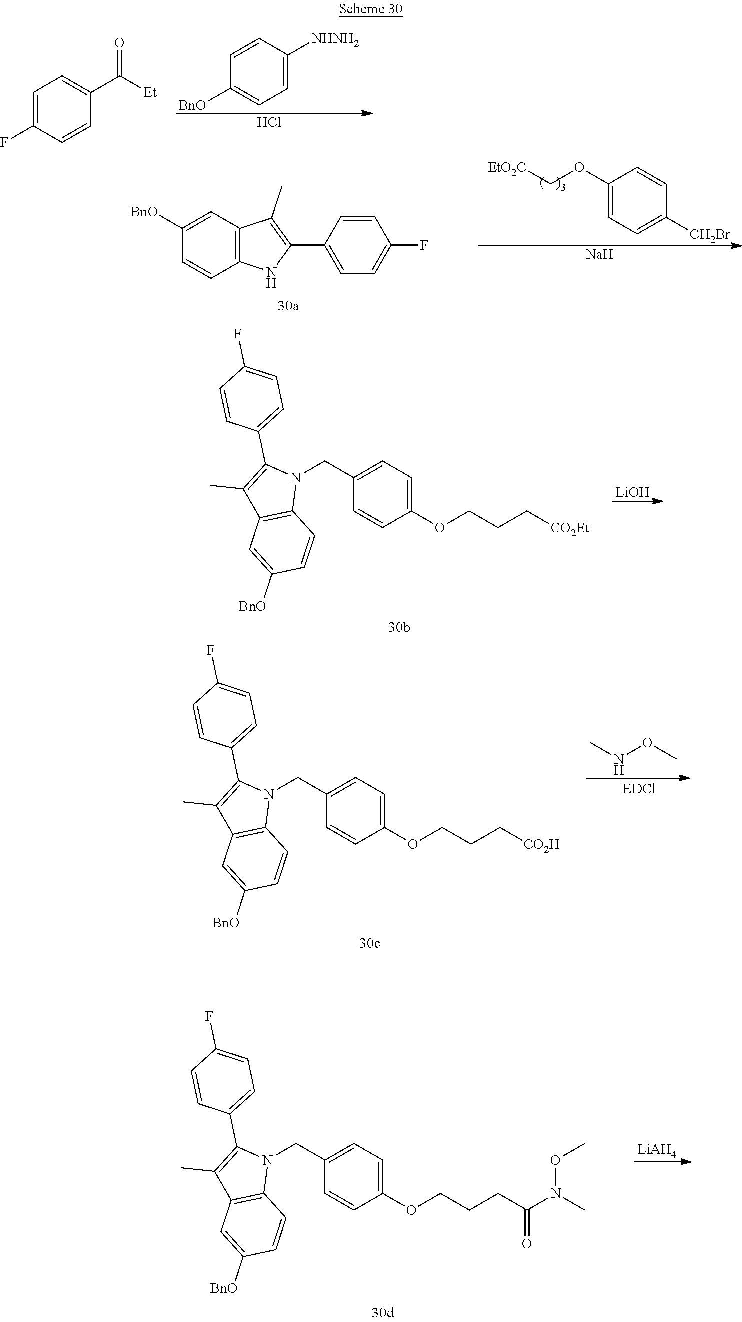

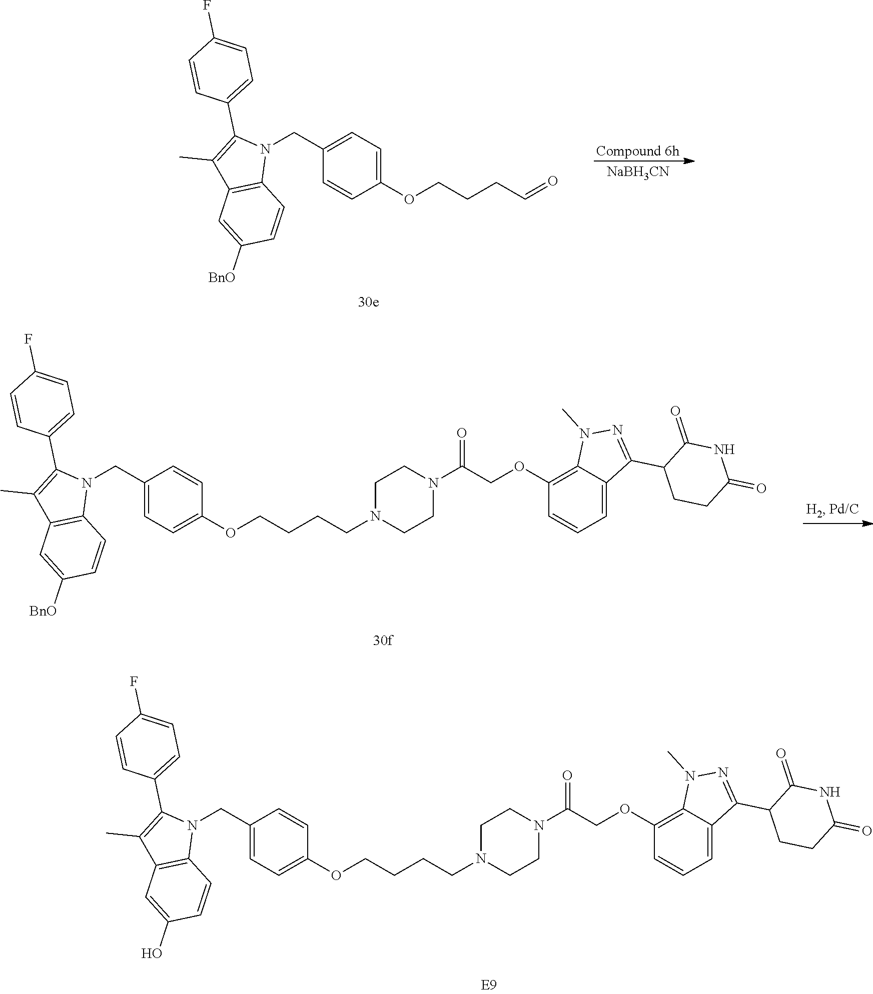 [SMILES: Cc1c(-c2ccc(F)cc2)n(Cc2ccc(OCCCC=O)cc2)c2ccc(OCc3ccccc3)cc12 ; "Compound 6h" ; "NaBH3CN" ; Cc1c(-c2ccc(F)cc2)n(Cc2ccc(OCCCCN3CCN(C(=O)COc4cccc5c(C6CCC(=O)NC6=O)nn(C)c45)CC3)cc2)c2ccc(OCc3ccccc3)cc12 ; Cc1c(-c2ccc(F)cc2)n(Cc2ccc(OCCCCN3CCN(C(=O)COc4cccc5c(C6CCC(=O)NC6=O)nn(C)c45)CC3)cc2)c2ccc(O)cc12]

Ethyl 4-(4-((5-(benzyloxy)-2-(4-fluorophenyl)-3-methyl-1H-indol-1-yl)methyl)-phenoxy)butanoate 30b. To a solution of compound 30a (770 mg, 2.33 mmol) in DMF (20 mL) was added NaH (79 mg, 3.45 mmol). After the mixture was stirred at 0° C. for 30 min, ethyl 4-(4-(bromomethyl)phenoxy)butanoate (666 mg, 2.33 mmol) was added. The reaction mixture was stirred at room temperature for 1 h, and then washed with H₂O (10 mL) and extracted with EtOAc (20 mL×2). The combined organic layers were dried over anhydrous Na₂SO₄, filtered, and concentrated to yield a crude product, which was purified by silica gel column chromatography eluting with EtOAc in PE to afford compound 30b (600 mg) in 47% yield. MS (ESI) m/z: 552.2 [M+H]⁺.

4-(4-((5-(Benzyloxy)-2-(4-fluorophenyl)-3-methyl-1H-indol-1-yl)methyl)-phenoxy)butanoic acid 30c. To a solution of compound 30b (600 mg, 1.09 mmol) in THE (4 mL) and MeOH (1 mL) was added LiOH (91.5 mg, 2.18 mmol) in H₂O (1 mL). After stirred for 8 h, the reaction mixture was concentrated, neutralized to pH 5 with 1N HCl, and extracted with EtOAc (10 mL×2). The combined organic layers were washed with brine, dried over anhydrous Na₂SO₄, filtered, and concentrated to afford compound 30c (560 mg) in a quantitative yield. MS (ESI) m/z: 524.2 [M+H]⁺.

4-(4-((5-(Benzyloxy)-2-(4-fluorophenyl)-3-methyl-1H-indol-1-yl)methyl)-phenoxy)-N-methoxy-N-methylbutanamide 30d. To a solution of compound 30c (560 mg, 1.1 mmol) and N,O-dimethylhydroxylamine (216 mg, 2.2 mmol) in DCM (10 mL) were added TEA (333 mg, 3.3 mmol), EDCI·HCl (315 mg, 1.65 mmol), and DMAP (13 mg, 0.11 mmol). After stirred for 8 h, the reaction was concentrated to yield a crude product, which was purified by silica gel column chromatography eluting with EtOAc in PE to afford compound 30d (500 mg) in 80% yield. MS (ESI) m/z: 567.3 [M+H]⁺.

4-(4-((5-(Benzyloxy)-2-(4-fluorophenyl)-3-methyl-1H-indol-1-yl)methyl)-phenoxy)butanal 30e. To a solution of compound 30d (200 mg, 0.35 mmol) in THE (4 mL) was added 1M LiAlH₄ in THE (0.7 mL, 0.70 mmol) dropwise under N₂ at −70° C. After stirred at −70° C. for 1 h, the reaction was quenched with sat. aqueous NH₄Cl (5 mL) and the reaction mixture was extracted with EtOAc (15 mL×3). The combined organic layers were dried over anhydrous Na₂SO₄, filtered, and concentrated to afford compound 30e as a crude compound, which was used directly in the next step without further purification. MS (ESI) m/z: 508.2 [M+H]⁺.

3-(7-(2-(4-(4-(4-((5-(Benzyloxy)-2-(4-fluorophenyl)-3-methyl-1H-indol-1-yl)-methyl)phenoxy)butyl)piperazin-1-yl)-2-oxoethoxy)-1-methyl-1H-indazol-3-yl)piperidine-2,6-dione 30f. To a solution of compound 30e (crude, 0.35 mmol) in DCM (4 mL) and MeOH (1 mL) was added 3-(1-methyl-7-(2-oxo-2-(piperazin-1-yl)ethoxy)-1H-indazol-3-yl)piperidine-2,6-dione 6h (135 mg, 0.35 mmol). After the mixture was stirred for 30 min, NaBH₃CN (44 mg, 0.7 mmol) was added. The reaction mixture was stirred for 8 h and then concentrated to yield a crude product, which was purified by silica gel column chromatography eluting with MeOH in DCM to afford compound 30f (222 mg) in 72% yield. MS (ESI) m/z: 877.4 [M+Na]⁺.

3-(7-(2-(4-(4-(4-((2-(4-Fluorophenyl)-5-hydroxy-3-methyl-1H-indol-1-yl)-methyl)phenoxy)butyl)piperazin-1-yl)-2-oxoethoxy)-1-methyl-1H-indazol-3-yl)piperidine-2,6-dione E9. To a solution of compound 30f (222 mg, 0.25 mmol) in MeOH (2 mL) and THE (2 mL) was added Pd/C (100 mg). After stirred under H₂ for 12 h, the reaction mixture was filtered and concentrated to yield a crude product, which was purified by reverse phase prep-HPLC to afford compound E9 (51.8 mg) in 26% yield. ¹H NMR (400 MHz, DMSO-d₆) δ 10.87 (s, 1H), 8.74 (s, 1H), 8.14 (s, 1H), 7.41-7.38 (m, 2H), 7.31 (t, J=9.2 Hz, 2H), 7.23 (d, J=8.0 Hz, 1H), 7.13 (d, J=8.8 Hz, 1H), 6.96 (t, J=7.6 Hz, 1H), 6.83-6.81 (m, 2H), 6.75-6.71 (m, 4H), 6.62 (dd, J=2.4, 8.8 Hz, 1H), 5.12 (s, 2H), 4.99 (s, 2H), 4.32 (dd, J=5.2, 10.0 Hz, 1H), 4.23 (s, 3H), 3.87 (t, J=6.4 Hz, 2H), 3.45-3.41 (m, 4H), 2.67-2.57 (m, 2H), 2.41-2.36 (m, 2H), 2.33-2.30 (m, 5H), 2.19-2.14 (m, 1H), 2.11 (s, 3H), 1.68-1.63 (m, 2H), 1.56-1.51 (m, 2H); MS (ESI) m/z: 787.2 [M+H]⁺.

### Example 31

### Preparation of 3-(6-(2-(4-((1-(4-((5-hydroxy-2-(4-hydroxyphenyl)-3-methyl-1H-indol-1-yl)-methyl)phenyl)piperidin-4-yl)methyl)piperazin-1-yl)-2-oxoethoxy)-1-methyl-1H-indazol-3-yl)-piperidine-2,6-dione E15

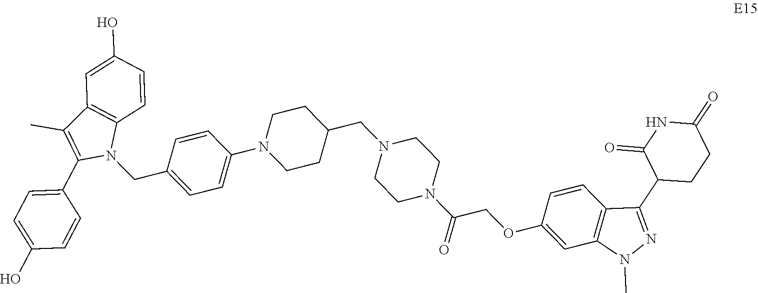 [SMILES: Cc1c(-c2ccc(O)cc2)n(Cc2ccc(N3CCC(CN4CCN(C(=O)COc5ccc6c(C7CCC(=O)NC7=O)nn(C)c6c5)CC4)CC3)cc2)c2ccc(O)cc12]

Compound E15 was prepared as shown in Scheme 31.

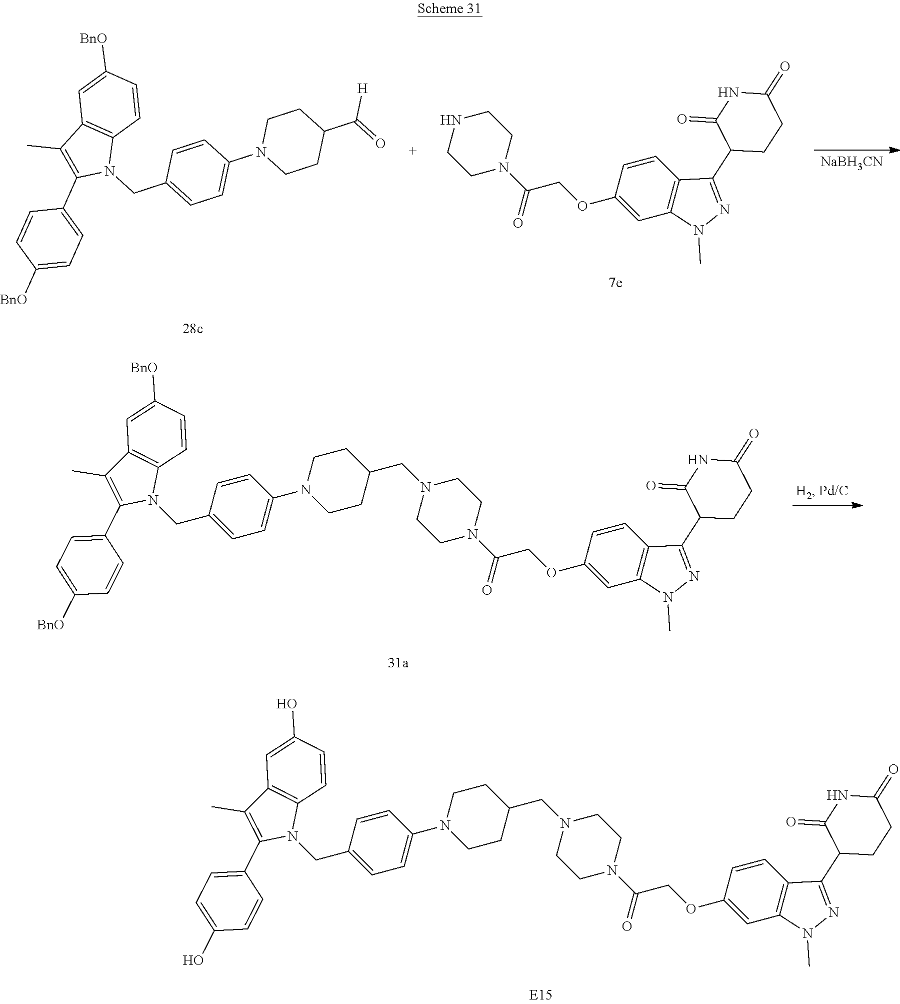 [SMILES: Cc1c(-c2ccc(OCc3ccccc3)cc2)n(Cc2ccc(N3CCC(C=O)CC3)cc2)c2ccc(OCc3ccccc3)cc12 ; Cn1nc(C2CCC(=O)NC2=O)c2ccc(OCC(=O)N3CCNCC3)cc21 ; "NaBH3CN" ; Cc1c(-c2ccc(OCc3ccccc3)cc2)n(Cc2ccc(N3CCC(CN4CCN(C(=O)COc5ccc6c(C7CCC(=O)NC7=O)nn(C)c6c5)CC4)CC3)cc2)c2ccc(OCc3ccccc3)cc12 ; Cc1c(-c2ccc(O)cc2)n(Cc2ccc(N3CCC(CN4CCN(C(=O)COc5ccc6c(C7CCC(=O)NC7=O)nn(C)c6c5)CC4)CC3)cc2)c2ccc(O)cc12]

3-(6-(2-(4-((1-(4-((5-(Benzyloxy)-2-(4-(benzyloxy)phenyl)-3-methyl-1H-indol-1-yl)methyl)phenyl)piperidin-4-yl)methyl)piperazin-1-yl)-2-oxoethoxy)-1-methyl-1H-indazol-3-yl)piperidine-2,6-dione 31a. To a solution of 1-(4-((5-(benzyloxy)-2-(4-(benzyloxy)phenyl)-3-methyl-1H-indol-1-yl)methyl)-phenyl)piperidine-4-carbaldehyde 28c (100 mg, 0.161 mmol) in DCM (4 mL) and MeOH (1 mL) at 0° C. were added 3-(1-methyl-6-(2-oxo-2-(piperazin-1-yl)ethoxy)-1H-indazol-3-yl)piperidine-2,6-dione 7e (62 mg, 0.161 mmol) and NaBH₃CN (20 mg, 0.322 mmol). After stirred at room temperature overnight, the reaction mixture was concentrated to yield a crude product, which was purified by silica gel column chromatography eluting with MeOH in DCM to afford compound 31a (131 mg) in 82% yield. MS (ESI) m/z: 990.5 [M+H]⁺.

3-(6-(2-(4-((1-(4-((5-Hydroxy-2-(4-hydroxyphenyl)-3-methyl-1H-indol-1-yl)-methyl)phenyl)piperidin-4-yl)methyl)piperazin-1-yl)-2-oxoethoxy)-1-methyl-1H-indazol-3-yl)-piperidine-2,6-dione E15. To a solution of compound 31a (131 mg, 0.132 mmol) in THE (30 mL) was added Pd/C (100 mg). After stirred for 48 h under H₂, the reaction mixture was filtered and concentrated to yield a crude product, which was purified with reverse phase prep-HPLC to afford compound E15 (25.5 mg) in 24% yield. ¹H NMR (400 MHz, DMSO-d₆) δ 10.86 (s, 1H), 9.68 (brs, 1H), 8.68 (brs, 1H), 7.57 (d, J=8.8 Hz, 1H), 7.17 (d, J=8.8 Hz, 2H), 7.07-7.04 (m, 2H), 6.86 (d, J=8.8 Hz, 2H), 6.80-6.68 (m, 6H), 6.56 (dd, J=2.4, 8.8 Hz, 1H), 5.06 (s, 2H), 4.86 (s, 2H), 4.30 (dd, J=2.4, 8.8 Hz, 1H), 3.91 (s, 3H), 3.60-3.53 (m, 4H), 2.69-2.53 (m, 5H), 2.42-2.29 (m, 5H), 2.20-2.13 (m, 3H), 2.10 (s, 3H), 2.05-1.94 (m, 1H), 1.74 (d, J=11.6 Hz, 2H), 1.66-1.53 (m, 1H), 1.20-1.09 (m, 2H); MS (ESI) m/z: 810.2 [M+H]⁺. crude

### Example 32

### Preparation of 3-(4-(2-(4-(4-(4-((5-hydroxy-2-(4-hydroxyphenyl)-3-methyl-1H-indol-1-yl)-methyl)phenoxy)butyl)piperazin-1-yl)-2-oxoethoxy)phenyl)piperidine-2,6-dione E24

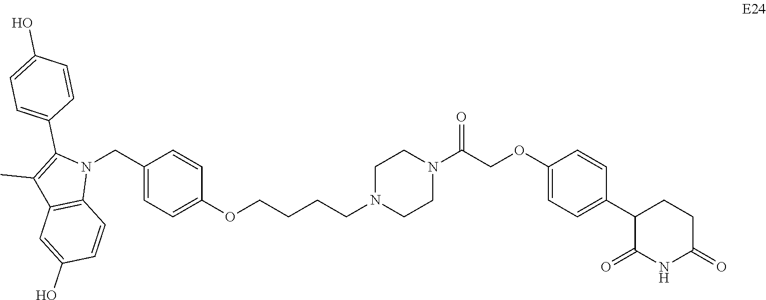 [SMILES: Cc1c(-c2ccc(O)cc2)n(Cc2ccc(OCCCCN3CCN(C(=O)COc4ccc(C5CCC(=O)NC5=O)cc4)CC3)cc2)c2ccc(O)cc12]

Compound E24 was prepared as shown in Scheme 32.

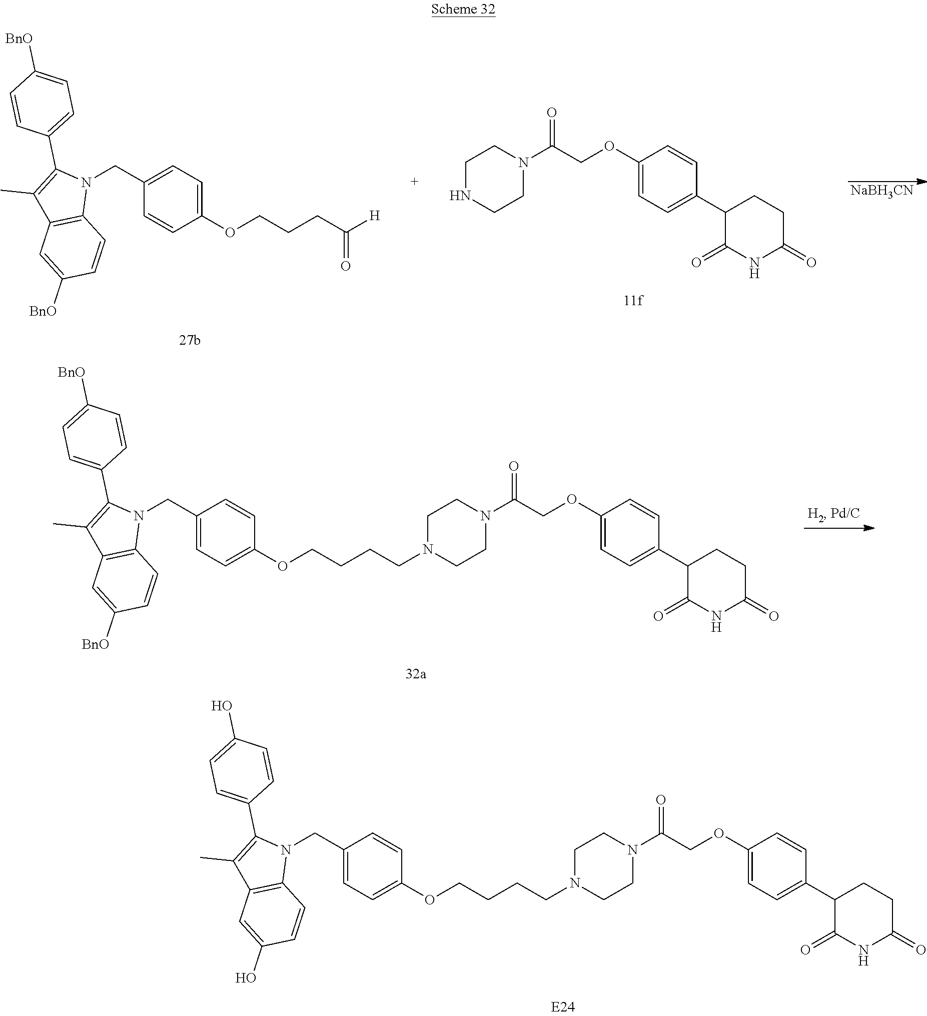 [SMILES: Cc1c(-c2ccc(OCc3ccccc3)cc2)n(Cc2ccc(OCCCC=O)cc2)c2ccc(OCc3ccccc3)cc12 ; O=C1CCC(c2ccc(OCC(=O)N3CCNCC3)cc2)C(=O)N1 ; "NaBH3CN" ; Cc1c(-c2ccc(OCc3ccccc3)cc2)n(Cc2ccc(OCCCCN3CCN(C(=O)COc4ccc(C5CCC(=O)NC5=O)cc4)CC3)cc2)c2ccc(OCc3ccccc3)cc12 ; Cc1c(-c2ccc(O)cc2)n(Cc2ccc(OCCCCN3CCN(C(=O)COc4ccc(C5CCC(=O)NC5=O)cc4)CC3)cc2)c2ccc(O)cc12]

3-(4-(2-(4-(4-(4-((5-(Benzyloxy)-2-(4-(benzyloxy)phenyl)-3-methyl-1H-indol-1-yl)methyl)phenoxy)butyl)piperazin-1-yl)-2-oxoethoxy)phenyl)piperidine-2,6-dione 32a. To a solution of 3-(4-(2-oxo-2-(piperazin-1-yl)ethoxy)phenyl)piperidine-2,6-dione hydrochloride 11f (100 mg, crude) in DCM (4 mL) and MeOH (1 mL) were added 4-(4-((5-(benzyloxy)-2-(4-(benzyloxy)phenyl)-3-methyl-1H-indol-1-yl)methyl)phenoxy)butanal 27b (90 mg, crude) and NaBH₃CN (20 mg, 0.33 mmol), followed by addition of AcOH (1 drop). After stirred for 8 h, the reaction mixture was concentrated to yield a crude product, which was purified by silica gel column chromatography eluting with MeOH in DCM to afford compound 32a (130 mg) in 87% yield. MS (ESI) m/z: 911.0 [M+H]⁺.

3-(4-(2-(4-(4-(4-((5-Hydroxy-2-(4-hydroxyphenyl)-3-methyl-1H-indol-1-yl)-methyl)phenoxy)butyl)piperazin-1-yl)-2-oxoethoxy)phenyl)piperidine-2,6-dione E24. To a solution of compound 32a (130 mg, 0.14 mmol) in THE (5 mL) and MeOH (5 mL) was added Pd/C (70 mg). After stirred under H₂ for 48 h, the reaction mixture was filtered and concentrated to yield a crude product, which was purified by reverse phase prep-HPLC to afford compound E24 (26.2 mg) in 26% yield. ¹H NMR (400 MHz, DMSO-d₆) δ 10.78 (s, 1H), 9.69 (s, 1H), 8.68 (s, 1H), 8.18 (s, 1H), 7.17-7.10 (m, 4H), 7.07-7.05 (m, 1H), 6.87-6.84 (m, 4H), 6.80 (d, J=13.2 Hz, 1H), 6.77-6.72 (m, 4H), 6.57 (dd, J=2.4, 8.8 Hz, 1H), 5.10 (s, 2H), 4.77 (s, 2H), 3.88 (t, J=6.0 Hz, 2H), 3.78 (dd, J=5.2, 11.6 Hz, 1H), 3.42 (t, J=4.4 Hz, 4H), 2.68-2.61 (m, 2H), 2.40-2.34 (m, 2H), 2.32-2.29 (m, 4H), 2.17-2.13 (m, 1H), 2.10 (s, 3H), 2.02-1.99 (m, 1H), 1.68-1.65 (m, 2H), 1.56-1.50 (m, 2H); MS (ESI) m/z: 731.5 [M+H]⁺.

### Example 33

### Preparation of 1-(4-(2-(4-(4-(4-((5-hydroxy-2-(4-hydroxyphenyl)-3-methyl-1H-indol-1-yl)-methyl)phenoxy)butyl)piperazin-1-yl)-2-oxoethoxy)phenyl)dihydropyrimidine-2,4(1H,3H)-dione E27

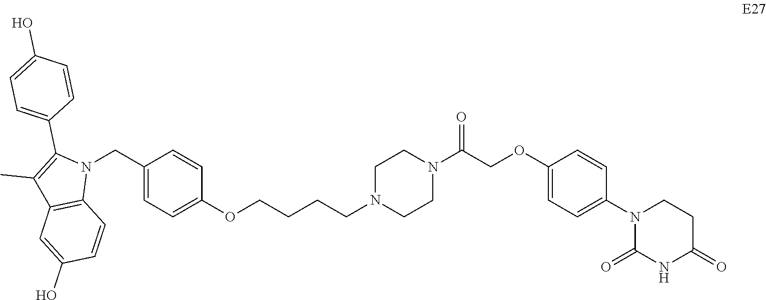 [SMILES: Cc1c(-c2ccc(O)cc2)n(Cc2ccc(OCCCCN3CCN(C(=O)COc4ccc(N5CCC(=O)NC5=O)cc4)CC3)cc2)c2ccc(O)cc12]

Compound E27 was prepared as shown in Scheme 33.

1-(4-(2-(4-(4-(4-((5-(Benzyloxy)-2-(4-(benzyloxy)phenyl)-3-methyl-1H-indol-1-yl)methyl)phenoxy)butyl)piperazin-1-yl)-2-oxoethoxy)phenyl)dihydropyrimidine-2,4(1H,3H)-dione 33a. To a solution of 4-(4-((5-(benzyloxy)-2-(4-(benzyloxy)phenyl)-3-methyl-1H-indol-1-yl)methyl)phenoxy)butanal 27b (170 mg, crude) in DCM (5 mL) and MeOH (0.5 mL) was added 1-(4-(2-oxo-2-(piperazin-1-yl)ethoxy)phenyl)dihydropyrimidine-2,4(1H,3H)-dione 15g (102 mg, 0.30 mmol), followed by addition of NaBH(OAc)₃ (130 mg, 0.60 mmol). After stirred for 12 h, the reaction mixture was concentrated to yield a crude product, which was purified by silica gel column chromatography eluting with MeOH in DCM to afford compound 33a (147 mg) in 53% yield. MS (ESI) m/z: 912.4 [M+H]⁺.

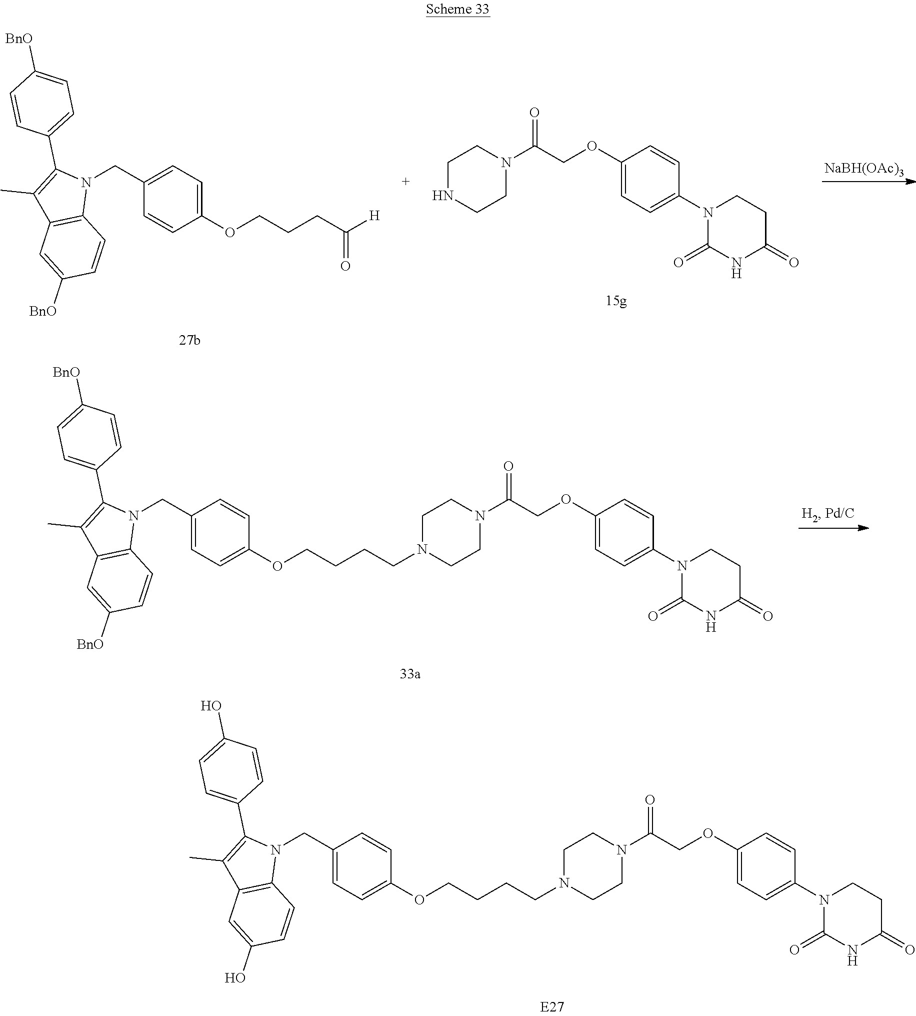 [SMILES: Cc1c(-c2ccc(OCc3ccccc3)cc2)n(Cc2ccc(OCCCC=O)cc2)c2ccc(OCc3ccccc3)cc12 ; O=C1CCN(c2ccc(OCC(=O)N3CCNCC3)cc2)C(=O)N1 ; Cc1c(-c2ccc(OCc3ccccc3)cc2)n(Cc2ccc(OCCCCN3CCN(C(=O)COc4ccc(N5CCC(=O)NC5=O)cc4)CC3)cc2)c2ccc(OCc3ccccc3)cc12 ; "NaBH(OAc)3" ; Cc1c(-c2ccc(O)cc2)n(Cc2ccc(OCCCCN3CCN(C(=O)COc4ccc(N5CCC(=O)NC5=O)cc4)CC3)cc2)c2ccc(O)cc12]

1-(4-(2-(4-(4-(4-((5-Hydroxy-2-(4-hydroxyphenyl)-3-methyl-1H-indol-1-yl)-methyl)phenoxy)butyl)piperazin-1-yl)-2-oxoethoxy)phenyl)dihydropyrimidine-2,4(1H,3H)-dione E27. To a solution of compound 33a (147 mg, 0.16 mmol) in MeOH (4 mL) and THE (4 mL) was added Pd/C (30 mg). After stirred under H₂ for 12 h, the reaction mixture was filtered and concentrated to yield a crude product, which was purified by reverse phase prep-HPLC to afford compound E27 (23.8 mg) in 20% yield. ¹H NMR (400 MHz, DMSO-d₆) δ 10.28 (s, 1H), 9.67 (brs, 1H), 8.71 (brs, 1H), 7.23-7.19 (m, 2H), 7.16-7.14 (m, 2H), 7.06 (d, J=8.8 Hz, 1H), 6.93-6.89 (m, 2H), 6.86-6.82 (m, 2H), 6.80 (d, J=2.4 Hz, 1H), 6.76-6.71 (m, 4H), 6.57 (dd, J=2.4, 8.8 Hz, 1H), 5.09 (s, 2H), 4.79 (s, 2H), 3.87 (t, J=6.4 Hz, 2H), 3.70 (t, J=6.4 Hz, 2H), 3.43-3.41 (m, 4H), 2.68 (t, J=6.8 Hz, 2H), 2.41-2.25 (m, 6H), 2.09 (s, 3H), 1.70-1.63 (m, 2H), 1.56-1.49 (m, 2H); MS (ESI) m/z: 732.5 [M+H]⁺.

The following compounds were prepared similarly according to the synthetic procedures or methodologies exemplified herein.

3-(6-Fluoro-4-(1-(6-(4-((1R,2S)-6-hydroxy-2-phenyl-1,2,3,4-tetrahydro-naphthalen-1-yl)phenoxy)hexyl)piperidin-4-yl)-1-oxoisoindolin-2-yl)piperidine-2,6-dione A2. ¹H NMR (400 MHz, DMSO-d₆) δ 11.02 (s, 1H), 10.02 (brs, 1H), 9.12 (s, 1H), 7.42-7.31 (m, 2H), 7.16-7.11 (m, 3H), 6.83 (d, J=6.8 Hz, 2H), 6.64-6.47 (m, 5H), 6.27 (d, J=8.8 Hz, 2H), 5.17 (dd, J=4.8, 12.8 Hz, 1H), 4.55-4.34 (m, 2H), 4.18 (d, J=4.8 Hz, 1H), 3.81 (t, J=4.8 Hz, 2H), 3.55-3.47 (m, 1H), 3.31-3.24 (m, 4H), 3.01-2.89 (m, 6H), 2.64-2.57 (m, 1H), 2.40-2.32 (m, 1H), 2.12-1.86 (m, 6H), 1.77-1.63 (m, 5H), 1.40-1.33 (m, 4H); MS (ESI) m/z: 744.5 [M+H]⁺.

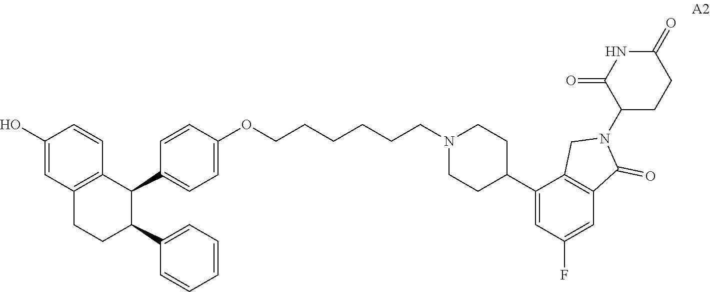 [SMILES: O=C1CCC(N2Cc3c(cc(F)cc3C3CCN(CCCCCCOc4ccc([C@@H]5c6ccc(O)cc6CC[C@@H]5c5ccccc5)cc4)CC3)C2=O)C(=O)N1]

3-(6-Fluoro-4-(1-((1-(4-((1R,2S)-6-hydroxy-2-phenyl-1,2,3,4-tetrahydro-naphthalen-1-yl)phenyl)piperidin-4-yl)methyl)piperidin-4-yl)-1-oxoisoindolin-2-yl)piperidine-2,6-dione A3. ¹H NMR (400 MHz, DMSO-d₆) δ 11.34 (s, 1H), 9.32 (s, 1H), 7.52-7.46 (m, 2H), 7.26-7.23 (m, 3H), 6.95 (d, J=6.8 Hz, 2H), 6.77-6.59 (m, 5H), 6.33 (d, J=8.4 Hz, 2H), 5.25 (dd, J=4.8, 12.8 Hz, 1H), 4.65-4.44 (m, 2H), 4.25 (d, J=4.0 Hz, 1H), 3.84-3.75 (m, 5H), 3.41-3.38 (m, 1H), 3.12-2.98 (m, 5H), 2.74-2.70 (m, 1H), 2.58-2.52 (m, 3H), 2.36 (d, J=6.8 Hz, 2H), 2.19-2.15 (m, 3H), 1.86-1.74 (m, 7H), 1.27-1.25 (m, 2H); MS (ESI) m/z: 741.5 [M+H]⁺.

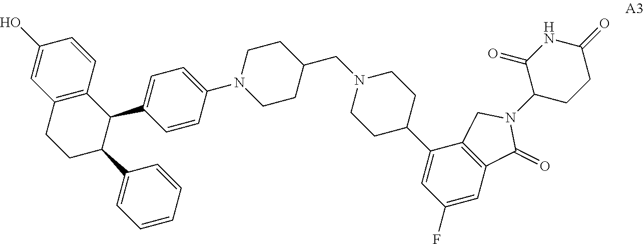 [SMILES: O=C1CCC(N2Cc3c(cc(F)cc3C3CCN(CC4CCN(c5ccc([C@@H]6c7ccc(O)cc7CC[C@@H]6c6ccccc6)cc5)CC4)CC3)C2=O)C(=O)N1]

3-(6-Fluoro-5-(1-(4-(4-((1R,2S)-6-hydroxy-2-phenyl-1,2,3,4-tetrahydro-naphthalen-1-yl)phenoxy)butyl)piperidin-4-yl)-1-oxoisoindolin-2-yl)piperidine-2,6-dione A4. ¹H NMR (400 MHz, DMSO-d₆) δ 10.99 (s, 1H), 9.12 (s, 1H), 7.60 (d, J=6.4 Hz, 1H), 7.46 (d, J=9.2 Hz, 1H), 7.16-7.10 (m, 3H), 6.82 (d, J=7.6 Hz, 2H), 6.63 (d, J=8.4 Hz, 1H), 6.61 (d, J=2.4 Hz, 1H), 6.53 (d, J=8.8 Hz, 2H), 6.48-6.47 (m, 1H), 6.26 (d, J=8.4 Hz, 2H), 5.10 (dd, J=5.2, 13.2 Hz, 1H), 4.43-4.26 (m, 2H), 4.17 (d, J=5.2 Hz, 1H), 3.84-3.82 (m, 2H), 2.99-2.87 (m, 6H), 2.61-2.57 (m, 1H), 2.41-2.37 (m, 1H), 2.36-2.31 (m, 2H), 2.20-2.10 (m, 1H), 2.05-1.91 (m, 4H), 1.72-1.64 (m, 7H), 1.56-1.54 (m, 2H); MS (ESI) m/z: 716.5 [M+H]⁺.

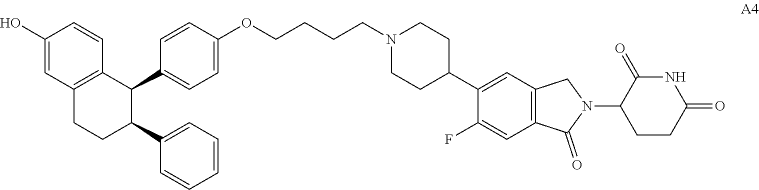 [SMILES: O=C1CCC(N2Cc3cc(C4CCN(CCCCOc5ccc([C@@H]6c7ccc(O)cc7CC[C@@H]6c6ccccc6)cc5)CC4)c(F)cc3C2=O)C(=O)N1]

3-(6-Fluoro-5-(1-(6-(4-((1R,2S)-6-hydroxy-2-phenyl-1,2,3,4-tetrahydro-naphthalen-1-yl)phenoxy)hexyl)piperidin-4-yl)-1-oxoisoindolin-2-yl)piperidine-2,6-dione A5. ¹H NMR (400 MHz, DMSO-d₆) δ 10.99 (s, 1H), 9.98 (s, 1H), 9.12 (s, 1H), 7.54-7.49 (m, 2H), 7.17-7.11 (m, 3H), 6.83 (d, J=6.8 Hz, 2H), 6.64-6.47 (m, 5H), 6.27 (d, J=8.8 Hz, 2H), 5.12 (dd, J=4.8, 12.8 Hz, 1H), 4.46-4.29 (m, 2H), 4.18 (d, J=4.8 Hz, 1H), 3.81 (t, J=4.8 Hz, 2H), 3.57-3.51 (m, 1H), 3.26-3.25 (m, 4H), 3.00-2.86 (m, 6H), 2.62-2.58 (m, 1H), 2.41-2.36 (m, 1H), 2.09-1.90 (m, 6H), 1.71-1.63 (m, 5H), 1.40-1.33 (m, 4H); MS (ESI) m/z: 744.5 [M+H]⁺.

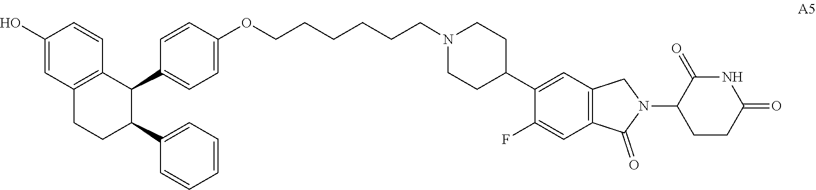 [SMILES: O=C1CCC(N2Cc3cc(C4CCN(CCCCCCOc5ccc([C@@H]6c7ccc(O)cc7CC[C@@H]6c6ccccc6)cc5)CC4)c(F)cc3C2=O)C(=O)N1]

3-(6-Fluoro-4-(1-(4-(4-((1S,2R)-6-hydroxy-2-phenyl-1,2,3,4-tetrahydro-naphthalen-1-yl)phenoxy)butyl)piperidin-4-yl)-1-oxoisoindolin-2-yl)piperidine-2,6-dione A12. ¹H NMR (400 MHz, DMSO-d₆) δ 10.99 (s, 1H), 9.11 (s, 1H), 7.39-7.35 (m, 2H), 7.17-7.08 (m, 3H), 6.81 (d, J=6.8 Hz, 2H), 6.64-6.61 (m, 2H), 6.53 (d, J=8.8 Hz, 2H), 6.49-6.46 (m, 1H), 6.26 (d, J=8.8 Hz, 2H), 5.13 (dd, J=4.8, 12.8 Hz, 1H), 4.52-4.31 (m, 2H), 4.17 (d, J=4.8 Hz, 1H), 3.86-3.82 (m, 2H), 3.31-3.29 (m, 2H), 3.02-2.87 (m, 5H), 2.67-2.58 (m, 2H), 2.49-2.32 (m, 2H), 2.12-1.99 (m, 4H), 1.74-1.63 (m, 7H), 1.58-1.55 (m, 2H); MS (ESI) m/z: 716.5 [M+H]⁺.

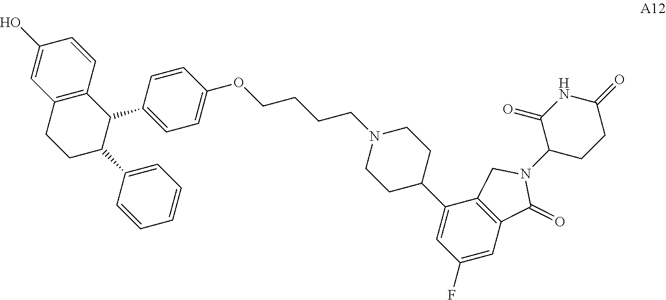

3-(6-Fluoro-5-(1-(4-(4-((1S,2R)-6-hydroxy-2-phenyl-1,2,3,4-tetrahydro-naphthalen-1-yl)phenoxy)butyl)piperidin-4-yl)-1-oxoisoindolin-2-yl)piperidine-2,6-dione A13. ¹H NMR (400 MHz, DMSO-d₆) δ 10.99 (s, 1H), 9.12 (s, 1H), 7.57 (s, 1H), 7.49 (d, J=9.2 Hz, 1H), 7.17-7.11 (m, 3H), 6.82 (d, J=6.8 Hz, 2H), 6.64-6.61 (m, 2H), 6.54 (d, J=8.8 Hz, 2H), 6.48 (dd, J=2.0, 8.4 Hz, 1H), 6.27 (d, J=8.8 Hz, 2H), 5.11 (dd, J=4.8, 13.2 Hz, 1H), 4.45-4.28 (m, 2H), 4.18 (d, J=4.8 Hz, 1H), 3.85-3.82 (m, 2H), 3.30-3.29 (m, 2H), 3.02-2.87 (m, 5H), 2.62-2.51 (m, 1H), 2.44-2.32 (m, 3H), 2.11-2.00 (m, 4H), 1.83-1.68 (m, 9H); MS (ESI) m/z: 716.5 [M+H]⁺.

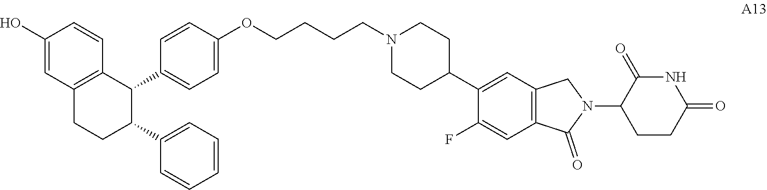 [SMILES: O=C1CCC(N2Cc3cc(C4CCN(CCCCOc5ccc([C@H]6c7ccc(O)cc7CC[C@H]6c6ccccc6)cc5)CC4)c(F)cc3C2=O)C(=O)N1]

3-(5-(4-((1-(4-((1R,2S)-6-Hydroxy-2-phenyl-1,2,3,4-tetrahydronaphthalen-1-yl)-phenyl)piperidin-4-yl)methyl)piperazin-1-yl)-1-oxoisoindolin-2-yl)piperidine-2,6-dione A14. ¹H NMR (400 MHz, DMSO-d₆) δ 10.93 (s, 1H), 9.10 (s, 1H), 7.52 (d, J=8.8 Hz, 1H), 7.16-7.10 (m, 3H), 7.05-7.03 (m, 2H), 6.84 (d, J=8.4 Hz, 2H), 6.59 (s, 1H), 6.52 (d, J=8.4 Hz, 2H), 6.48 (d, J=8.0 Hz, 1H), 6.20 (d, J=8.4 Hz, 2H), 5.06 (dd, J=5.2, 13.6 Hz, 1H), 4.34-4.18 (m, 2H), 4.11 (d, J=4.4 Hz, 1H), 3.52-3.51 (m, 4H), 3.28-3.21 (m, 6H), 2.97-2.86 (m, 4H), 2.63-2.60 (m, 1H), 2.38-2.33 (m, 1H), 2.19 (d, J=6.4 Hz, 2H), 2.09-1.95 (m, 2H), 1.76-1.65 (m, 4H), 1.23-1.14 (m, 4H); MS (ESI) m/z: 724.5 [M+H]⁺.

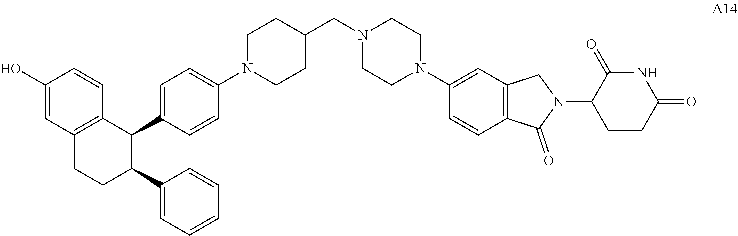 [SMILES: O=C1CCC(N2Cc3cc(N4CCN(CC5CCN(c6ccc([C@@H]7c8ccc(O)cc8CC[C@@H]7c7ccccc7)cc6)CC5)CC4)ccc3C2=O)C(=O)N1]

(R)-3-(5-(4-((1-(4-((1R,2S)-6-Hydroxy-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione A15. ¹H NMR (400 MHz, DMSO-d₆) δ 10.93 (s, 1H), 9.11 (s, 1H), 7.52 (d, J=7.6 Hz, 1H), 7.14-7.05 (m, 5H), 6.84 (d, J=6.8 Hz, 2H), 6.65-6.47 (m, 5H), 6.21 (d, J=8.8 Hz, 2H), 5.05 (dd, J=4.8, 12.8 Hz, 1H), 4.34-4.13 (m, 3H), 3.52-3.49 (m, 2H), 3.28-3.25 (m, 6H), 2.95-2.86 (m, 3H), 2.60-2.55 (m, 2H), 2.50-2.34 (m, 5H), 2.19 (d, J=6.8 Hz, 2H), 2.12-1.96 (m, 2H), 1.75-1.61 (m, 4H), 1.20-1.14 (m, 2H); MS (ESI) m/z: 724.5 [M+H]⁺.

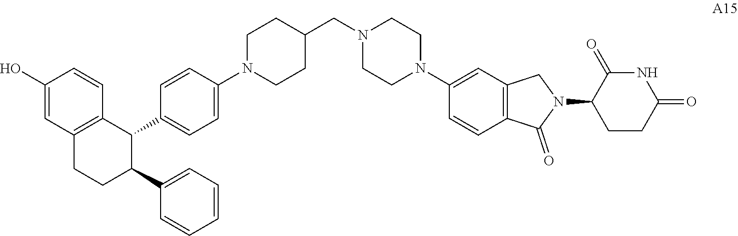 [SMILES: O=C1CC[C@@H](N2Cc3cc(N4CCN(CC5CCN(c6ccc([C@H]7c8ccc(O)cc8CC[C@@H]7c7ccccc7)cc6)CC5)CC4)ccc3C2=O)C(=O)N1]

(S)-3-(5-(4-((1-(4-((1R,2S)-6-Hydroxy-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione A16. ¹H NMR (400 MHz, DMSO-d₆) δ 10.93 (s, 1H), 9.08 (s, 1H), 7.52 (d, J=7.6 Hz, 1H), 7.16-7.03 (m, 5H), 6.84 (d, J=6.8 Hz, 2H), 6.64 (d, J=8.4 Hz, 1H), 6.60 (d, J=2.4 Hz, 1H), 6.52 (d, J=8.8 Hz, 2H), 6.48 (d, J=2.4, 8.4 Hz, 1H), 6.21 (d, J=8.8 Hz, 2H), 5.05 (dd, J=4.8, 12.8 Hz, 1H), 4.34-4.18 (m, 2H), 4.13 (d, J=4.8 Hz, 1H), 3.51-3.49 (m, 2H), 3.28-3.27 (m, 6H), 2.96-2.86 (m, 3H), 2.60-2.55 (m, 1H), 2.49-2.39 (m, 5H), 2.38-2.33 (m, 1H), 2.19 (d, J=6.8 Hz, 2H), 2.12-1.94 (m, 2H), 1.76-1.61 (m, 4H), 1.19-1.14 (m, 2H); MS (ESI) m/z: 724.2 [M+H]⁺.

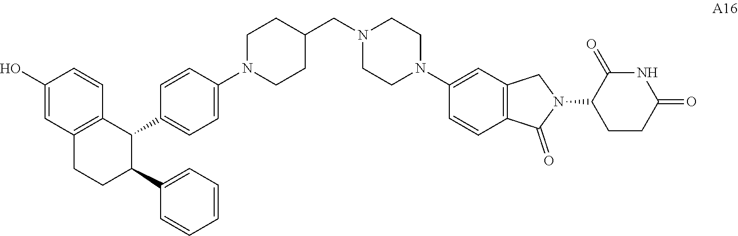

3-(1-((4-(((5-(4-((1R,2S)-6-Hydroxy-2-phenyl-1,2,3,4-tetrahydronaphthalen-1-yl)phenoxy)pentyl)amino)methyl)phenoxy)methyl)-4-oxo-4H-thieno[3,4-c]pyrrol-5(6H)-yl)piperidine-2,6-dione A17. ¹H NMR (400 MHz, DMSO-d₆) δ 8.25 (s, 1H), 8.01 (s, 1H), 7.29 (d, J=8.4 Hz, 2H), 7.16-7.08 (m, 3H), 6.99 (d, J=8.8 Hz, 2H), 6.82 (d, J=6.4 Hz, 2H), 6.64-6.60 (m, 2H), 6.52-6.46 (m, 3H), 6.25 (d, J=8.4 Hz, 2H), 5.29 (s, 2H), 5.01 (dd, J=5.2, 13.2 Hz, 1H), 4.36-4.20 (m, 2H), 4.17 (d, J=5.2 Hz, 1H), 3.80-3.76 (m, 5H), 3.01-2.84 (m, 4H), 2.61-2.56 (m, 3H), 2.35-2.30 (m, 1H), 2.05-1.96 (m, 2H), 1.71-1.58 (m, 3H), 1.53-1.46 (m, 2H), 1.38-1.33 (m, 2H), 1.26-1.23 (m, 2H); MS (ESI) m/z: 770.4 [M+H]⁺.

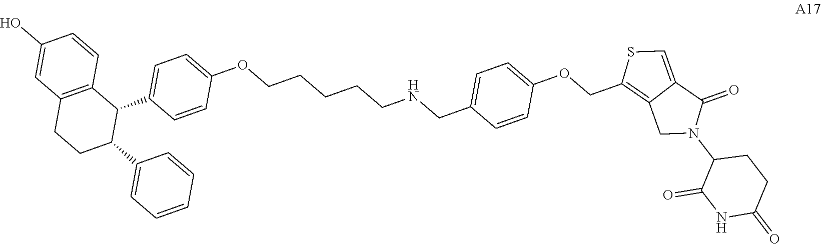 [SMILES: O=C1CCC(N2Cc3c(csc3COc3ccc(CNCCCCCOc4ccc([C@H]5c6ccc(O)cc6CC[C@H]5c5ccccc5)cc4)cc3)C2=O)C(=O)N1]

3-(1-(1-(6-(4-((1R,2S)-6-Hydroxy-2-phenyl-1,2,3,4-tetrahydronaphthalen-1-yl)-phenoxy)hexyl)piperidin-4-yl)-4-oxo-4H-thieno[3,4-c]pyrrol-5(6H)-yl)piperidine-2,6-dione A18. ¹H NMR (400 MHz, DMSO-d₆) δ 10.95 (s, 1H), 9.10 (brs, 1H), 8.16 (s, 1H), 7.81 (s, 1H), 7.16-7.10 (m, 3H), 6.82 (d, J=6.4 Hz, 2H), 6.63 (d, J=8.4 Hz, 1H), 6.60 (d, J=2.4 Hz, 1H), 6.51 (d, J=8.8 Hz, 2H), 6.47 (dd, J=2.8, 8.4 Hz, 1H), 6.25 (d, J=8.8 Hz, 2H), 5.00 (dd, J=5.2, 13.6 Hz, 1H), 4.38-4.18 (m, 2H), 4.15 (d, J=2.8 Hz, 1H), 3.79 (t, J=6.4 Hz, 2H), 3.29-3.21 (m, 2H), 2.97-2.80 (m, 6H), 2.59-2.54 (m, 1H), 2.36-2.27 (m, 3H), 2.09-1.92 (m, 6H), 1.71-1.57 (m, 5H), 1.44-1.24 (m, 7H); MS (ESI) m/z: 732.2 [M+H]⁺.

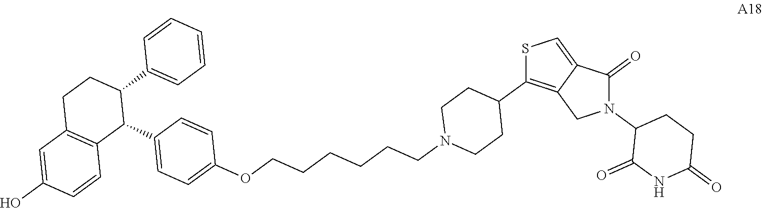 [SMILES: O=C1CCC(N2Cc3c(csc3C3CCN(CCCCCCOc4ccc([C@@H]5c6ccc(O)cc6CC[C@@H]5c5ccccc5)cc4)CC3)C2=O)C(=O)N1]

3-(1-(1-(4-(4-((1R,2S)-6-Hydroxy-2-phenyl-1,2,3,4-tetrahydronaphthalen-1-yl)-phenoxy)butyl)piperidin-4-yl)-4-oxo-4H-thieno[3,4-c]pyrrol-5(6H)-yl)piperidine-2,6-dione A19. ¹H NMR (400 MHz, DMSO-d₆) δ 10.96 (s, 1H), 9.18 (s, 1H), 8.25 (s, 1H), 7.81 (s, 1H), 7.16-7.11 (m, 3H), 6.84-6.81 (m, 2H), 6.63 (d, J=8.0 Hz, 1H), 6.60 (d, J=2.4 Hz, 1H), 6.52 (d, J=8.8 Hz, 2H), 6.47 (dd J=2.4, 8.0 Hz, 1H), 6.25 (d, J=8.0 Hz, 2H), 5.00 (dd, J=5.2, 13.2 Hz, 1H), 4.37-4.17 (m, 2H), 4.16 (s, 1H), 3.84-3.81 (m, 2H), 3.32-3.28 (m, 2H), 2.96-2.75 (m, 6H), 2.59-2.55 (m, 1H), 2.38-2.28 (m, 3H), 2.08-2.07 (m, 1H), 1.95-1.92 (m, 5H), 1.71-1.50 (m, 7H); MS (ESI) m/z: 704.4 [M+H]⁺.

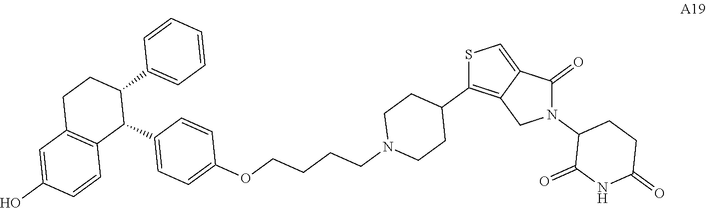 [SMILES: O=C1CCC(N2Cc3c(csc3C3CCN(CCCCOc4ccc([C@@H]5c6ccc(O)cc6CC[C@@H]5c5ccccc5)cc4)CC3)C2=O)C(=O)N1]

3-(2-(1-(4-(4-((1R,2S)-6-Hydroxy-2-phenyl-1,2,3,4-tetrahydronaphthalen-1-yl)-phenoxy)butyl)piperidin-4-yl)-4-oxo-4,6-dihydro-5H-thieno[2,3-c]pyrrol-5-yl)piperidine-2,6-dione A20. ¹H NMR (400 MHz, DMSO-d₆) δ 10.94 (s, 1H), 9.13 (s, 1H), 7.17-7.11 (m, 3H), 7.03 (s, 1H), 6.83 (d, J=6.8 Hz, 2H), 6.64 (d, J=8.8 Hz, 1H), 6.61 (d, J=2.4 Hz, 1H), 6.53 (d, J=8.4 Hz, 2H), 6.48 (dd, J=2.4, 8.0 Hz, 1H), 6.27 (d, J=8.8 Hz, 2H), 4.98 (dd, J=5.2, 13.2 Hz, 1H), 4.50-4.33 (m, 2H), 4.18 (d, J=4.8 Hz, 1H), 3.84-3.81 (m, 2H), 3.43-3.31 (m, 3H), 3.06-2.80 (m, 6H), 2.67-2.56 (m, 3H), 2.40-2.28 (m, 1H), 2.10-2.07 (m, 3H), 1.99-1.96 (m, 1H), 1.87-1.78 (m, 2H), 1.76-1.68 (m, 5H); MS (ESI) m/z: 704.2 [M+H]⁺.

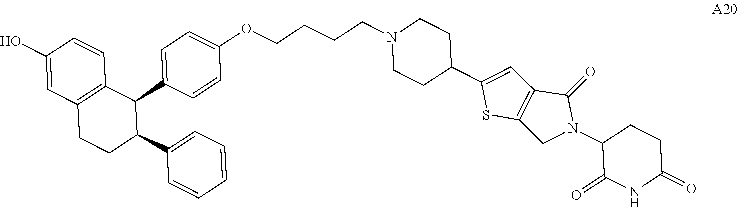 [SMILES: O=C1CCC(N2Cc3sc(C4CCN(CCCCOc5ccc([C@@H]6c7ccc(O)cc7CC[C@@H]6c6ccccc6)cc5)CC4)cc3C2=O)C(=O)N1]

3-(2-(1-(6-(4-((1R,2S)-6-Hydroxy-2-phenyl-1,2,3,4-tetrahydronaphthalen-1-yl)-phenoxy)hexyl)piperidin-4-yl)-4-oxo-4,6-dihydro-5H-thieno[2,3-c]pyrrol-5-yl)piperidine-2,6-dione A21. ¹H NMR (400 MHz, DMSO-d₆) δ 10.94 (s, 1H), 9.12 (s, 1H), 7.17-7.10 (m, 3H), 6.98 (s, 1H), 6.82 (d, J=7.2 Hz, 2H), 6.63 (d, J=8.8 Hz, 2H), 6.47 (dd, J=2.0, 8.0 Hz, 1H), 6.25 (d, J=8.8 Hz, 2H), 4.98 (dd, J=5.2, 13.2 Hz, 1H), 4.48-4.30 (m, 2H), 4.17 (d, J=4.8 Hz, 1H), 3.79 (t, J=6.0 Hz, 2H), 2.97-2.81 (m, 6H), 2.59-2.55 (m, 1H), 2.35-2.30 (m, 1H), 2.28-2.24 (m, 2H), 2.10-2.02 (m, 1H), 1.96-1.90 (m, 4H), 1.71-1.59 (m, 5H), 1.44-1.26 (m, 8H); MS (ESI) m/z: 732.1 [M+H]⁺.

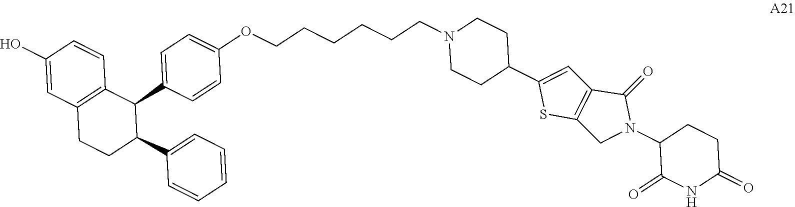 [SMILES: O=C1CCC(N2Cc3sc(C4CCN(CCCCCCOc5ccc([C@@H]6c7ccc(O)cc7CC[C@@H]6c6ccccc6)cc5)CC4)cc3C2=O)C(=O)N1]

3-((3-((2-(4-((1-(4-((1R,2S)-6-Hydroxy-2-phenyl-1,2,3,4-tetrahydronaphthalen-1-yl)phenyl)piperidin-4-yl)methyl)piperazin-1-yl)ethyl)amino)phenyl)amino)piperidine-2,6-dione B15. ¹H NMR (400 MHz, DMSO-d₆) δ 10.75 (s, 1H), 9.10 (s, 1H), 7.20-7.13 (m, 3H), 6.84-6.75 (m, 3H), 6.65-6.60 (m, 2H), 6.56-6.47 (m, 3H), 6.19 (d, J=8.0 Hz, 2H), 5.91-5.86 (m, 3H), 5.47 (d, J=7.2 Hz, 1H), 4.99 (s, 1H), 4.24-4.18 (m, 1H), 4.12 (d, J=4.0 Hz, 1H), 3.50-3.47 (m, 2H), 3.06-3.03 (m, 2H), 2.96-2.90 (m, 2H), 2.72-2.68 (m, 1H), 2.63-2.59 (m, 1H), 2.38-2.32 (m, 4H), 2.12-2.06 (m, 4H), 2.03-1.96 (m, 2H), 1.88-1.79 (m, 1H), 1.72-1.69 (m, 3H), 1.62-1.52 (m, 1H), 1.49-1.42 (m, 1H), 1.30-1.20 (m, 6H), 1.15-1.08 (m, 2H); MS (ESI) m/z: 727.6 [M+H]⁺.

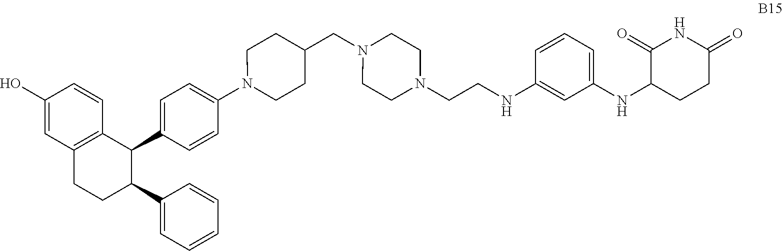 [SMILES: O=C1CCC(Nc2cccc(NCCN3CCN(CC4CCN(c5ccc([C@@H]6c7ccc(O)cc7CC[C@@H]6c6ccccc6)cc5)CC4)CC3)c2)C(=O)N1]

N-(3-((2,6-Dioxopiperidin-3-yl)amino)phenyl)-2-(4-((1-(4-((1R,2S)-6-hydroxy-2-phenyl-1,2,3,4-tetrahydronaphthalen-1-yl)phenyl)piperidin-4-yl)methyl)piperazin-1-yl)-N-methylacetamide B20. ¹H NMR (400 MHz, DMSO-d₆) δ 10.78 (s, 1H), 9.07 (s, 1H), 8.20 (s, 1H), 7.15-7.08 (m, 4H), 6.82 (d, J=6.4 Hz, 2H), 6.63 (d, J=8.4 Hz, 1H), 6.59-6.57 (m, 2H), 6.50 (d, J=8.8 Hz, 2H), 6.46 (dd, J=2.8, 8.4 Hz, 2H), 6.19 (d, J=8.4 Hz, 2H), 6.04 (d, J=7.2 Hz, 1H), 4.38-4.32 (m, 1H), 4.11 (d, J=4.8 Hz, 1H), 3.48-3.37 (m, 4H), 3.26-3.22 (m, 2H), 3.09 (s, 3H), 2.99-2.85 (m, 4H), 2.77-2.66 (m, 1H), 2.59-2.54 (m, 1H), 2.42-2.26 (m, 8H), 2.07-2.06 (m, 2H), 2.00-1.95 (m, 1H), 1.89-1.82 (m, 1H), 1.71-1.66 (m, 3H), 1.53-1.50 (m, 1H), 1.12-1.04 (m, 2H); MS (ESI) m/z: 755.5 [M+H]⁺.

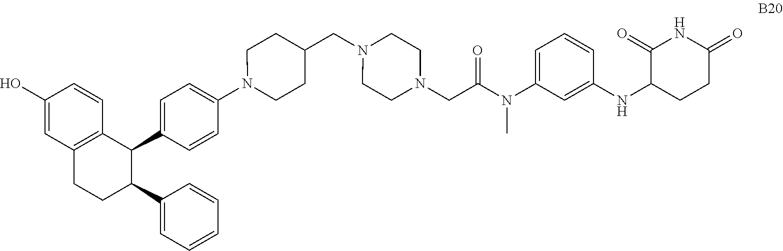 [SMILES: CN(C(=O)CN1CCN(CC2CCN(c3ccc([C@@H]4c5ccc(O)cc5CC[C@@H]4c4ccccc4)cc3)CC2)CC1)c1cccc(NC2CCC(=O)NC2=O)c1]

(S)-3-(6-(2-(4-(4-(4-((1R,2S)-6-Hydroxy-2-phenyl-1,2,3,4-tetrahydronaphthalen-1-yl)phenoxy)butyl)piperazin-1-yl)-2-oxoethoxy)-1-methyl-1H-indazol-3-yl)piperidine-2,6-dione B23. ¹H NMR (400 MHz, DMSO-d₆) δ 10.86 (s, 1H), 9.11 (s, 1H), 7.57 (d, J=8.8 Hz, 1H), 7.17-7.11 (m, 3H), 7.04 (d, J=2.0 Hz, 1H), 6.82 (d, J=6.8 Hz, 2H), 6.77 (dd, J=2.0, 9.2 Hz, 1H), 6.63 (d, J=8.0 Hz, 1H), 6.60 (d, J=2.4 Hz, 1H), 6.52 (d, J=8.8 Hz, 2H), 6.48 (dd, J=2.0, 8.0 Hz, 1H), 6.25 (d, J=8.8 Hz, 2H), 4.86 (s, 2H), 4.30 (dd, J=5.2, 9.6 Hz, 1H), 4.17 (d, J=4.8 Hz, 1H), 3.91 (s, 3H), 3.83 (t, J=6.4 Hz, 2H), 3.46 (s, 4H), 3.29-3.28 (m, 1H), 3.02-2.89 (m, 2H), 2.68-2.57 (m, 2H), 2.40-2.37 (m, 2H), 2.33-2.30 (m, 5H), 2.20-2.03 (m, 2H), 1.73-1.62 (m, 3H), 1.56-1.49 (m, 2H); MS (ESI) m/z: 756.2 [M+H]⁺.

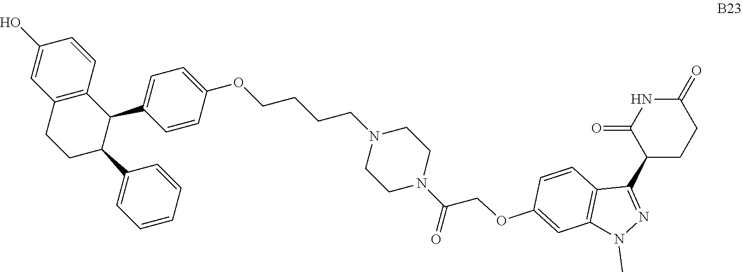 [SMILES: Cn1nc([C@@H]2CCC(=O)NC2=O)c2ccc(OCC(=O)N3CCN(CCCCOc4ccc([C@@H]5c6ccc(O)cc6CC[C@@H]5c5ccccc5)cc4)CC3)cc21]

3-(6-(2-(4-(4-(4-((1S,2R)-6-Hydroxy-2-phenyl-1,2,3,4-tetrahydronaphthalen-1-yl)phenoxy)butyl)piperazin-1-yl)-2-oxoethoxy)-1-methyl-1H-indazol-3-yl)piperidine-2,6-dione B24. ¹H NMR (400 MHz, DMSO-d₆) δ 10.87 (s, 1H), 7.57 (d, J=8.8 Hz, 1H), 7.16-7.09 (m, 3H), 7.05 (s, 1H), 6.82 (d, J=8.4 Hz, 2H), 6.78 (dd, J=1.6, 9.2 Hz, 1H), 6.65-6.61 (m, 2H), 6.53-6.47 (m, 3H), 6.26 (d, J=8.4 Hz, 2H), 4.87 (s, 2H), 4.30 (dd, J=4.8, 9.2 Hz, 1H), 4.17 (d, J=4.8 Hz, 1H), 3.91 (s, 3H), 3.83 (t, J=6.0 Hz, 2H), 3.64-3.46 (m, 4H), 3.30 (dd, J=3.6, 12.4 Hz, 1H), 3.02-2.88 (m, 2H), 2.68-2.57 (m, 2H), 2.40-2.33 (m, 2H), 2.31-2.30 (m, 5H), 2.18-2.06 (m, 2H), 1.71-1.62 (m, 3H), 1.56-1.51 (m, 2H); MS (ESI) m/z: 756.5 [M+H]⁺.

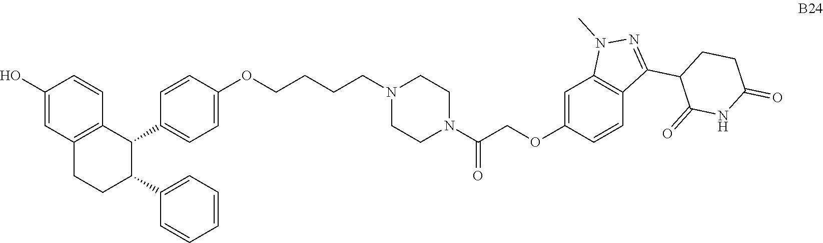 [SMILES: Cn1nc(C2CCC(=O)NC2=O)c2ccc(OCC(=O)N3CCN(CCCCOc4ccc([C@H]5c6ccc(O)cc6CC[C@H]5c5ccccc5)cc4)CC3)cc21]

3-(6-(4-((1-(4-((1R,2S)-6-Hydroxy-2-phenyl-1,2,3,4-tetrahydronaphthalen-1-yl)-phenyl)piperidin-4-yl)methyl)piperazin-1-yl)-1-methyl-1H-indazol-3-yl)piperidine-2,6-dione B25. ¹H NMR (400 MHz, DMSO-d₆) δ 10.83 (s, 1H), 8.20 (s, 1H), 7.48 (d, J=8.8 Hz, 1H), 7.19-7.05 (m, 3H), 6.90 (dd, J=2.4, 9.2 Hz, 1H), 6.86-6.79 (m, 3H), 6.54 (d, J=8.4 Hz, 1H), 6.61 (d, J=2.4 Hz, 1H), 6.53 (d, J=8.4 Hz, 2H), 6.47 (dd, J=2.4, 8.0 Hz, 1H), 6.20 (d, J=8.8 Hz, 2H), 4.25 (dd, J=5.2, 9.2 Hz, 1H), 4.12 (d, J=4.8 Hz, 1H), 3.88 (s, 3H), 3.54-3.46 (m, 3H), 3.29-3.16 (m, 8H), 2.97-2.86 (m, 2H), 2.67-2.58 (m, 2H), 2.40-2.24 (m, 2H), 2.21-1.97 (m, 5H), 1.80-1.61 (m, 4H), 1.20-1.11 (m, 2H); MS (ESI) m/z: 723.3 [M+H]⁺.

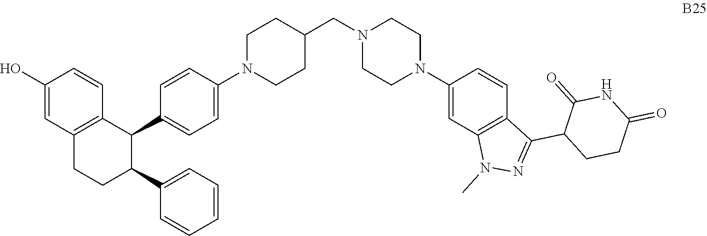 [SMILES: Cn1nc(C2CCC(=O)NC2=O)c2ccc(N3CCN(CC4CCN(c5ccc([C@@H]6c7ccc(O)cc7CC[C@@H]6c6ccccc6)cc5)CC4)CC3)cc21]

(R)-3-(6-(2-(4-(4-(4-((1R,2S)-6-Hydroxy-2-phenyl-1,2,3,4-tetrahydronaphthalen-1-yl)phenoxy)butyl)piperazin-1-yl)-2-oxoethoxy)-1-methyl-1H-indazol-3-yl)piperidine-2,6-dione B26. ¹H NMR (400 MHz, DMSO-d₆) δ 10.86 (s, 1H), 9.11 (s, 1H), 7.57 (d, J=8.8 Hz, 1H), 7.17-7.11 (m, 3H), 7.04 (s, 1H), 6.82 (d, J=6.8 Hz, 2H), 6.77 (d, J=8.4 Hz, 1H), 6.65-6.61 (m, 2H), 6.51 (d, J=8.4 Hz, 2H), 6.48 (d, J=8.0 Hz, 1H), 6.26 (d, J=8.8 Hz, 2H), 4.86 (s, 2H), 4.30 (dd, J=5.2, 9.6 Hz, 1H), 4.17 (d, J=4.8 Hz, 1H), 3.91 (s, 3H), 3.83 (t, J=6.4 Hz, 2H), 3.46 (s, 4H), 3.29-3.28 (m, 1H), 3.02-2.89 (m, 2H), 2.68-2.57 (m, 2H), 2.40-2.37 (m, 2H), 2.33-2.30 (m, 5H), 2.20-2.03 (m, 2H), 1.72-1.49 (m, 5H); MS (ESI) m/z: 756.2 [M+H]⁺.

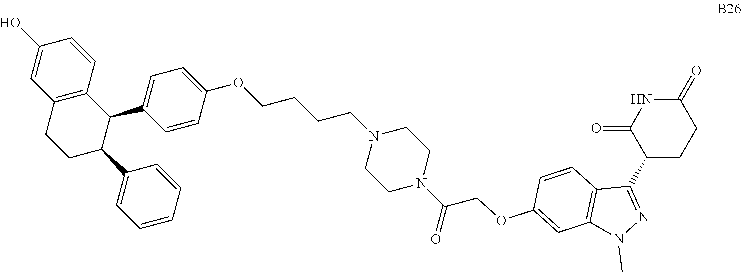 [SMILES: Cn1nc([C@H]2CCC(=O)NC2=O)c2ccc(OCC(=O)N3CCN(CCCCOc4ccc([C@@H]5c6ccc(O)cc6CC[C@@H]5c5ccccc5)cc4)CC3)cc21]

3-(6-(2-(4-((1-(4-((1R,2S)-6-Hydroxy-2-phenyl-1,2,3,4-tetrahydronaphthalen-1-yl)phenyl)piperidin-4-yl)methyl)piperazin-1-yl)-2-oxoethoxy)-1-methyl-1H-indazol-3-yl)-piperidine-2,6-dione B27. ¹H NMR (400 MHz, DMSO-d₆) δ 10.88 (s, 1H), 9.11 (s, 1H), 8.16 (s, 1H), 7.57 (d, J=8.4 Hz, 1H), 7.16-7.05 (m, 4H), 6.83 (d, J=6.8 Hz, 2H), 6.77 (dd, J=1.6, 8.8 Hz, 1H), 6.64 (d, J=8.4 Hz, 1H), 6.60-6.59 (m, 1H), 6.53 (d, J=8.4 Hz, 2H), 6.47 (dd, J=2.4, 8.4 Hz, 1H), 6.20 (d, J=8.8 Hz, 2H), 4.87 (s, 2H), 4.30 (dd, J=5.2, 9.6 Hz, 1H), 4.12 (d, J=4.8 Hz, 1H), 3.91 (s, 3H), 3.51-3.48 (m, 7H), 3.34-3.27 (m, 2H), 3.01-2.86 (m, 2H), 2.70-2.60 (m, 2H), 2.50-2.40 (m, 2H), 2.32-2.31 (m, 3H), 2.17-2.04 (m, 4H), 1.75-1.72 (m, 3H), 1.69-1.60 (m, 1H), 1.17-1.09 (m, 2H); MS (ESI) m/z: 781.2 [M+H]⁺.

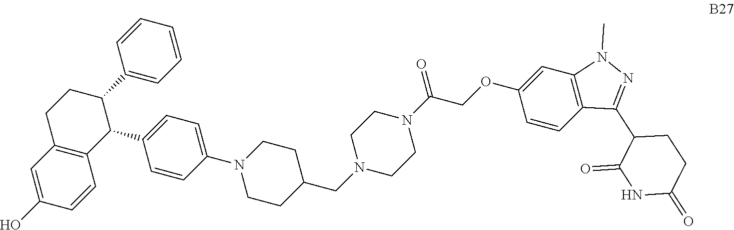 [SMILES: Cn1nc(C2CCC(=O)NC2=O)c2ccc(OCC(=O)N3CCN(CC4CCN(c5ccc([C@@H]6c7ccc(O)cc7CC[C@@H]6c6ccccc6)cc5)CC4)CC3)cc21]

3-(6-(2-(4-(3-(1-(4-((1R,2S)-6-Hydroxy-2-phenyl-1,2,3,4-tetrahydronaphthalen-1-yl)phenyl)piperidin-4-yl)propyl)piperazin-1-yl)-2-oxoethoxy)-1-methyl-1H-indazol-3-yl)-piperidine-2,6-dione B28. ¹H NMR (400 MHz, DMSO-d₆) δ 10.86 (s, 1H), 9.09 (brs, 1H), 8.16 (s, 1H), 7.57 (d, J=8.8 Hz, 1H), 7.16-7.08 (m, 3H), 7.04 (d, J=2.0 Hz, 1H), 6.83 (d, J=6.4 Hz, 2H), 6.77 (dd, J=2.0 Hz, 9.2 Hz, 1H), 6.63 (d, J=8.4 Hz, 1H), 6.59 (d, J=2.4 Hz, 1H), 6.51 (d, J=8.4 Hz, 2H), 6.47 (dd, J=2.4, 8.4 Hz, 1H), 6.19 (d, J=8.4 Hz, 2H), 4.86 (s, 2H), 4.30 (dd, J=4.8 Hz, 9.6 Hz, 1H), 4.12 (d, J=4.8 Hz, 1H), 3.91 (s, 3H), 3.55-3.40 (m, 6H), 3.27 (dd, J=2.8 Hz, 12.4 Hz, 2H), 3.03-2.85 (m, 2H), 2.70-2.54 (m, 2H), 2.48-2.37 (m, 3H), 2.37-2.22 (m, 5H), 2.21-2.02 (m, 2H), 1.74-1.59 (m, 3H), 1.54-1.39 (m, 2H), 1.36-1.06 (m, 5H); MS (ESI) m/z: 809.3 [M+H]⁺.

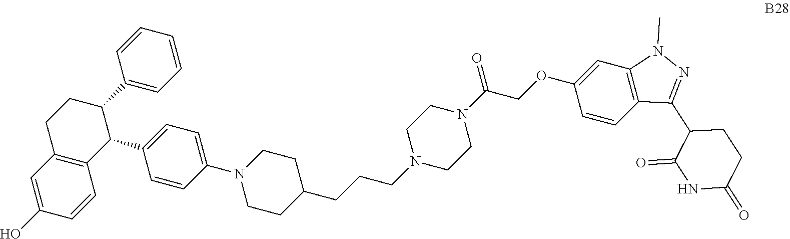 [SMILES: Cn1nc(C2CCC(=O)NC2=O)c2ccc(OCC(=O)N3CCN(CCCC4CCN(c5ccc([C@@H]6c7ccc(O)cc7CC[C@@H]6c6ccccc6)cc5)CC4)CC3)cc21]

3-(6-(4-(2-(4-((1R,2S)-6-Hydroxy-2-phenyl-1,2,3,4-tetrahydronaphthalen-1-yl)-phenoxy)ethyl)piperazin-1-yl)-1-methyl-1H-indazol-3-yl)piperidine-2,6-dione B29. ¹H NMR (400 MHz, DMSO-d₆) δ 10.83 (s, 1H), 9.20 (brs, 1H), 8.15 (s, 1H), 7.48 (d, J=8.8 Hz, 1H), 7.17-7.09 (m, 3H), 6.92-6.82 (m, 4H), 6.65-6.55 (m, 4H), 6.48 (dd, J=2.4 Hz, 8.4 Hz, 1H), 6.27 (d, J=8.4 Hz, 2H), 4.25 (dd, J=4.8 Hz, 8.8 Hz, 1H), 4.18 (d, J=4.8 Hz, 1H), 3.98 (t, J=5.6 Hz, 2H), 3.88 (s, 3H), 3.21-3.19 (m, 5H), 3.01-2.91 (m, 2H), 2.69 (t, J=5.6 Hz, 2H), 2.64-2.55 (m, 6H), 2.32-2.26 (m, 1H), 2.18-2.07 (m, 2H), 1.73-1.68 (m, 1H); MS (ESI) m/z: 670.2 [M+H]⁺.

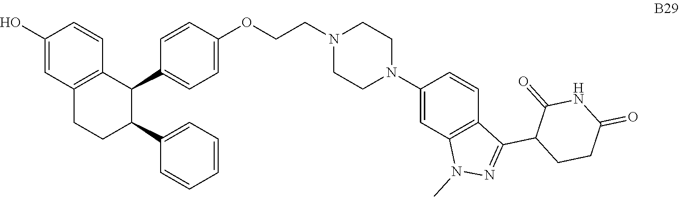 [SMILES: Cn1nc(C2CCC(=O)NC2=O)c2ccc(N3CCN(CCOc4ccc([C@@H]5c6ccc(O)cc6CC[C@@H]5c5ccccc5)cc4)CC3)cc21]

3-(6-((1-(4-(4-((1R,2S)-6-Hydroxy-2-phenyl-1,2,3,4-tetrahydronaphthalen-1-yl)-phenoxy)butyl)piperidin-4-yl)oxy)-1-methyl-1H-indazol-3-yl)piperidine-2,6-dione B30. ¹H NMR (400 MHz, DMSO-d₆) δ 10.84 (s, 1H), 9.11 (brs, 1H), 8.19 (s, 1H), 7.54 (d, J=8.8 Hz, 1H), 7.16-7.12 (m, 3H), 7.08 (d, J=2.0 Hz, 1H), 6.82 (d, J=6.8 Hz, 2H), 6.73 (dd, J=2.0 Hz, 8.8 Hz, 1H), 6.65-6.60 (m, 2H), 6.53 (d, J=8.8 Hz, 2H), 6.47 (dd, J=2.4 Hz, 8.0 Hz, 1H), 6.26 (d, J=8.8 Hz, 2H), 4.50-4.48 (m, 1H), 4.28 (dd, J=4.8 Hz, 9.2 Hz, 1H), 4.17 (d, J=4.8 Hz, 1H), 3.91 (s, 3H), 3.83 (t, J=6.4 Hz, 2H), 2.97-2.95 (m, 2H), 2.70-2.59 (m, 4H), 2.34-2.30 (m, 4H), 2.24-2.19 (m, 3H), 2.02-1.95 (m, 4H), 1.66-1.63 (m, 4H), 1.54-1.50 (m, 2H); MS (ESI) m/z: 713.2 [M+H]⁺.

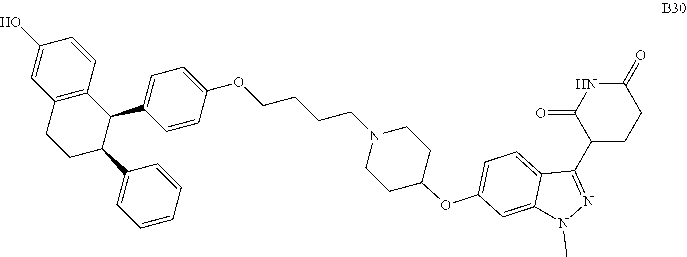 [SMILES: Cn1nc(C2CCC(=O)NC2=O)c2ccc(OC3CCN(CCCCOc4ccc([C@@H]5c6ccc(O)cc6CC[C@@H]5c5ccccc5)cc4)CC3)cc21]

3-(6-(4-(3-(1-(4-((1R,2S)-6-Hydroxy-2-phenyl-1,2,3,4-tetrahydronaphthalen-1-yl)phenyl)piperidin-4-yl)propyl)piperazin-1-yl)-1-methyl-1H-indazol-3-yl)piperidine-2,6-dione B31. ¹H NMR (400 MHz, DMSO-d₆) δ 10.84 (s, 1H), 9.12 (s, 1H), 8.18 (s, 1H), 7.49 (d, J=8.8 Hz, 1H), 7.16-7.05 (m, 3H), 6.91 (d, J=9.2 Hz, 1H), 6.83-6.82 (m, 3H), 6.64 (d, J=8.0 Hz, 1H), 6.59 (d, J=1.6 Hz, 1H), 6.52 (d, J=8.0 Hz, 2H), 6.47 (dd, J=2.4, 8.4 Hz, 1H), 6.19 (d, J=8.4 Hz, 2H), 4.25 (dd, J=5.2, 9.6 Hz, 1H), 4.12 (d, J=4.4 Hz, 1H), 3.88 (s, 3H), 3.51-3.49 (m, 4H), 3.30-3.21 (m, 7H), 3.00-2.90 (m, 2H), 2.67-2.59 (m, 2H), 2.47-2.44 (m, 2H), 2.33-2.29 (m, 2H), 2.18-2.06 (m, 2H), 1.71-1.68 (m, 3H), 1.51-1.48 (m, 2H), 1.30-1.11 (m, 6H); MS (ESI) m/z: 751.3 [M+H]⁺.

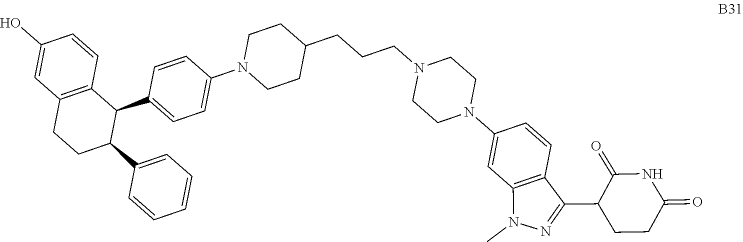 [SMILES: Cn1nc(C2CCC(=O)NC2=O)c2ccc(N3CCN(CCCC4CCN(c5ccc([C@@H]6c7ccc(O)cc7CC[C@@H]6c6ccccc6)cc5)CC4)CC3)cc21]

3-(6-((1-(2-(4-((1R,2S)-6-Hydroxy-2-phenyl-1,2,3,4-tetrahydronaphthalen-1-yl)-phenoxy)ethyl)piperidin-4-yl)oxy)-1-methyl-1H-indazol-3-yl)piperidine-2,6-dione B32. ¹H NMR (400 MHz, DMSO-d₆) δ 10.85 (s, 1H), 9.13 (brs, 1H), 8.16 (s, 1H), 7.54 (d, J=8.8 Hz, 1H), 7.17-7.07 (m, 4H), 6.83 (d, J=6.8 Hz, 2H), 6.73 (dd, J=2.0 Hz, 9.2 Hz, 1H), 6.65-6.61 (m, 2H), 6.54 (d, J=8.8 Hz, 2H), 6.48 (dd, J=2.4 Hz, 8.0 Hz, 1H), 6.27 (d, J=8.8 Hz, 2H), 4.52-4.49 (m, 1H), 4.28 (dd, J=5.2 Hz, 9.6 Hz, 1H), 4.17 (d, J=5.2 Hz, 1H), 3.94-3.91 (m, 5H), 3.02-2.91 (m, 3H), 2.78-2.76 (m, 2H), 2.67-2.56 (m, 4H), 2.38-2.28 (m, 3H), 2.17-1.97 (m, 4H), 1.73-1.61 (m, 3H); MS (ESI) m/z: 685.2 [M+H]⁺.

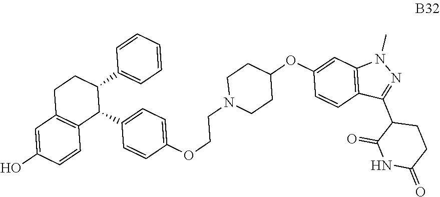 [SMILES: Cn1nc(C2CCC(=O)NC2=O)c2ccc(OC3CCN(CCOc4ccc([C@@H]5c6ccc(O)cc6CC[C@@H]5c5ccccc5)cc4)CC3)cc21]

3-(6-(2-(4-(2-(4-((1R,2S)-6-Hydroxy-2-phenyl-1,2,3,4-tetrahydronaphthalen-1-yl)phenoxy)ethyl)piperazin-1-yl)-2-oxoethoxy)-1-methyl-1H-indazol-3-yl)piperidine-2,6-dione B33. ¹H NMR (400 MHz, DMSO-d₆) δ 10.85 (s, 1H), 9.11 (brs, 1H), 7.57 (d, J=8.8 Hz, 1H), 7.17-7.10 (m, 3H), 7.04 (d, J=2.0 Hz, 1H), 6.83 (d, J=6.8 Hz, 2H), 6.77 (dd, J=2.0 Hz, 8.8 Hz, 1H), 6.64 (d, J=8.0 Hz, 1H), 6.61 (d, J=2.4 Hz, 1H), 6.56 (d, J=8.8 Hz, 2H), 6.48 (dd, J=2.8 Hz, 8.4 Hz, 1H), 6.27 (d, J=8.8 Hz, 2H), 4.86 (s, 2H), 4.30 (dd, J=5.2 Hz, 9.6 Hz, 1H), 4.18 (d, J=4.8 Hz, 1H), 3.94 (t, J=5.6 Hz, 2H), 3.90 (s, 3H), 3.46-3.42 (m, 4H), 2.97-2.91 (m, 2H), 2.67-2.59 (m, 4H), 2.58-2.51 (m, 3H), 2.43 (s, 2H), 2.34-2.29 (m, 1H), 2.18-2.06 (m, 2H), 1.73-1.69 (m, 1H); MS (ESI) m/z: 728.2 [M+H]⁺.

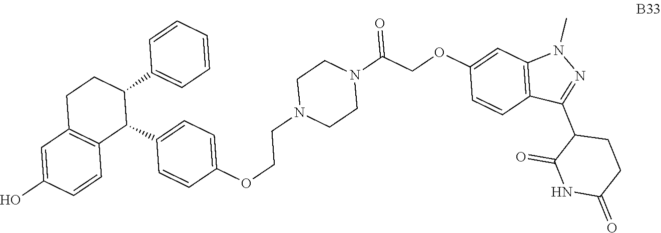 [SMILES: Cn1nc(C2CCC(=O)NC2=O)c2ccc(OCC(=O)N3CCN(CCOc4ccc([C@@H]5c6ccc(O)cc6CC[C@@H]5c5ccccc5)cc4)CC3)cc21]

3-(6-(2-(4-(4-(4-((1R,2S)-6-Hydroxy-2-phenyl-1,2,3,4-tetrahydronaphthalen-1-yl)phenoxy)butyl)piperidin-1-yl)-2-oxoethoxy)-1-methyl-1H-indazol-3-yl)piperidine-2,6-dione B34. ¹H NMR (400 MHz, DMSO-d₆) δ 10.85 (s, 1H), 9.10 (s, 1H), 7.56 (d, J=8.8 Hz, 1H), 7.16-7.10 (m, 3H), 7.03 (d, J=1.6 Hz, 1H), 6.82 (d, J=6.4 Hz, 2H), 6.76 (dd, J=2.0 Hz, 8.0 Hz, 1H), 6.63 (d, J=8.4 Hz, 1H), 6.60 (d, J=2.4 Hz, 1H), 6.51 (d, J=8.8 Hz, 2H), 6.48 (dd, J=2.4 Hz, 8.4 Hz, 1H), 6.25 (d, J=8.4 Hz, 2H), 4.84 (d, J=4.4 Hz, 2H), 4.33 (s, 1H), 4.29 (dd, J=5.2 Hz, 9.2 Hz, 1H), 4.17 (d, J=4.8 Hz, 1H), 3.90 (s, 3H), 3.89-3.83 (m, 1H), 3.80 (t, J=6.4 Hz, 2H), 3.04-2.91 (m, 3H), 2.65-2.56 (m, 3H), 2.35-2.27 (m, 1H), 2.19-2.09 (m, 1H), 2.07-1.92 (m, 1H), 1.77-1.56 (m, 5H), 1.56-1.32 (m, 3H), 1.32-1.18 (m, 3H), 1.18-1.04 (m, 1H), 1.01-0.88 (m, 1H); MS (ESI) m/z: 755.2 [M+H]⁺.

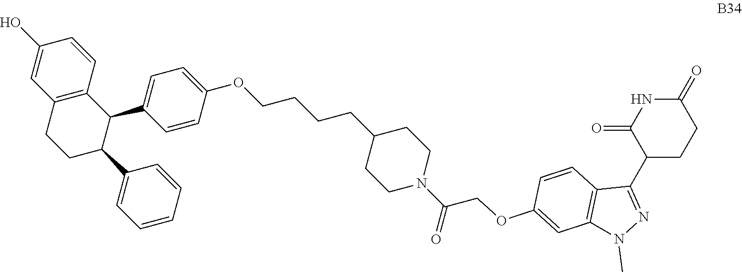 [SMILES: Cn1nc(C2CCC(=O)NC2=O)c2ccc(OCC(=O)N3CCC(CCCCOc4ccc([C@@H]5c6ccc(O)cc6CC[C@@H]5c5ccccc5)cc4)CC3)cc21]

3-(6-((1-(4-(4-((1R,2S)-6-Hydroxy-2-phenyl-1,2,3,4-tetrahydronaphthalen-1-yl)-phenoxy)butanoyl)piperidin-4-yl)oxy)-1-methyl-1H-indazol-3-yl)piperidine-2,6-dione B35. ¹H NMR (400 MHz, DMSO-d₆) δ 10.86 (s, 1H), 9.11 (s, 1H), 7.56 (d, J=8.8 Hz, 1H), 7.06-7.10 (m, 4H), 6.82 (d, J=6.8 Hz, 2H), 6.75 (d, J=8.4 Hz, 1H), 6.63 (d, J=8.0 Hz, 1H), 6.60 (d, J=2.0 Hz, 1H), 6.53 (d, J=8.4 Hz, 2H), 6.47 (dd, J=2.4 Hz, 8.4 Hz, 1H), 6.26 (d, J=8.8 Hz, 2H), 4.74-4.71 (m, 1H), 4.29 (dd, J=5.2 Hz, 9.2 Hz, 1H), 4.17 (d, J=4.8 Hz, 1H), 3.92 (s, 3H), 3.88-3.82 (m, 2H), 3.72-3.68 (m, 1H), 3.32-3.23 (m, 2H), 2.97-2.91 (m, 2H), 2.65-2.61 (m, 2H), 2.45 (t, J=7.2 Hz, 2H), 2.33-2.29 (m, 1H), 2.18-1.97 (m, 5H), 1.87 (t, J=6.8 Hz, 2H), 1.71-1.52 (m, 4H); MS (ESI) m/z: 727.2 [M+H]⁺.

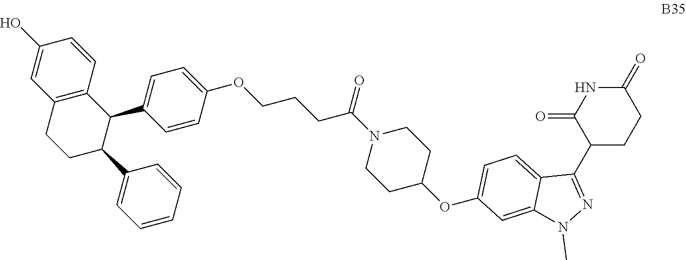 [SMILES: Cn1nc(C2CCC(=O)NC2=O)c2ccc(OC3CCN(C(=O)CCCOc4ccc([C@@H]5c6ccc(O)cc6CC[C@@H]5c5ccccc5)cc4)CC3)cc21]

3-(6-(2-(4-(2-(1-(4-((1R,2S)-6-Hydroxy-2-phenyl-1,2,3,4-tetrahydronaphthalen-1-yl)phenyl)piperidin-4-yl)ethyl)piperazin-1-yl)-2-oxoethoxy)-1-methyl-1H-indazol-3-yl)-piperidine-2,6-dione B36. ¹H NMR (400 MHz, DMSO-d₆) δ 10.87 (s, 1H), 9.10 (brs, 1H), 8.16 (s, 1H), 7.57 (d, J=8.8 Hz, 1H), 7.24-7.07 (m, 3H), 7.05-7.03 (m, 1H), 6.84-6.80 (m, 2H), 6.77 (dd, J=2.4 Hz, 8.8 Hz, 1H), 6.64 (d, J=8.4 Hz, 1H), 6.59 (d, J=2.0 Hz, 1H), 6.52 (d, J=8.8 Hz, 2H), 6.47 (dd, J=2.4 Hz, 8.8 Hz, 1H), 6.19 (d, J=9.2 Hz, 2H), 4.86 (s, 2H), 4.30 (dd, J=4.8 Hz, 9.6 Hz, 1H), 4.12 (d, J=4.8 Hz, 1H), 3.91 (s, 3H), 3.55-3.41 (m, 6H), 3.27-3.21 (m, 2H), 3.05-2.89 (m, 1H), 2.86 (d, J=6.8 Hz, 1H), 2.71-2.55 (m, 1H), 2.48-2.39 (m, 3H), 2.37-2.25 (m, 4H), 2.21-1.94 (m, 3H), 1.69 (d, J=7.6 Hz, 3H), 1.45-1.30 (m, 3H), 1.21-1.07 (m, 2H)c; MS (ESI) m/z: 795.3 [M+H]⁺.

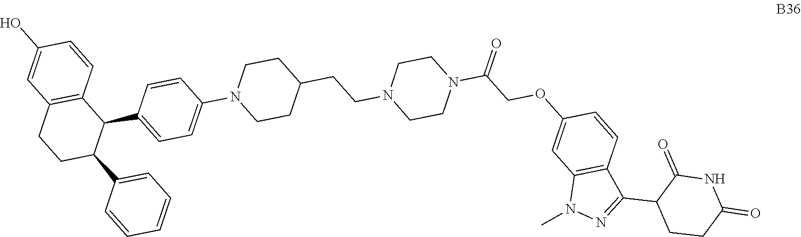 [SMILES: Cn1nc(C2CCC(=O)NC2=O)c2ccc(OCC(=O)N3CCN(CCC4CCN(c5ccc([C@@H]6c7ccc(O)cc7CC[C@@H]6c6ccccc6)cc5)CC4)CC3)cc21]

3-(6-(4-(2-(1-(4-((1R,2S)-6-Hydroxy-2-phenyl-1,2,3,4-tetrahydronaphthalen-1-yl)phenyl)piperidin-4-yl)ethyl)piperazin-1-yl)-1-methyl-1H-indazol-3-yl)piperidine-2,6-dione B37. ¹H NMR (400 MHz, DMSO-d₆) δ 10.83 (s, 1H), 9.08 (brs, 1H), 8.16 (s, 1H), 7.49 (d, J=8.8 Hz, 1H), 7.22-7.07 (m, 3H), 6.92 (dd, J=1.6, 9.2 Hz, 1H), 6.90-6.82 (m, 3H), 6.64 (d, J=8.4 Hz, 1H), 6.59 (d, J=2.4 Hz, 1H), 6.52 (d, J=8.8 Hz, 2H), 6.47 (d, J=2.4, 8.4 Hz, 1H), 6.19 (d, J=8.8 Hz, 2H), 4.26 (dd, J=4.8, 8.8 Hz, 1H), 4.12 (d, J=4.8 Hz, 1H), 3.88 (s, 3H), 3.49 (d, J=10.8 Hz, 2H), 3.27-3.24 (m, 1H), 3.24-3.10 (m, 4H), 3.02-2.84 (m, 2H), 2.68-2.58 (m, 2H), 2.57-2.53 (m, 4H), 2.45-2.41 (m, 1H), 2.37 (t, J=6.4 Hz, 2H), 2.34-2.23 (m, 1H), 2.20-1.94 (m, 3H), 1.77-1.63 (m, 3H), 1.50-1.33 (m, 3H), 1.22-1.12 (m, 2H); MS (ESI) m/z: 737.2 [M+H]⁺.

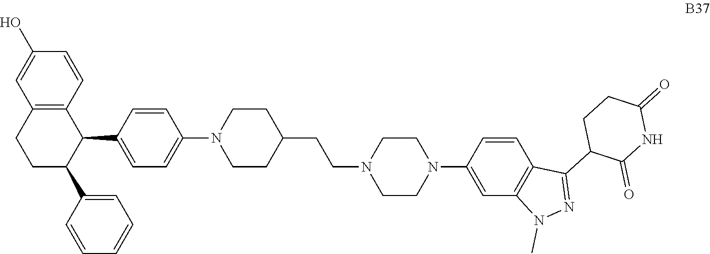 [SMILES: Cn1nc(C2CCC(=O)NC2=O)c2ccc(N3CCN(CCC4CCN(c5ccc([C@@H]6c7ccc(O)cc7CC[C@@H]6c6ccccc6)cc5)CC4)CC3)cc21]

3-(6-(2-(4-(4-(4-((1R,2S)-6-Hydroxy-2-phenyl-1,2,3,4-tetrahydronaphthalen-1-yl)phenoxy)butyl)piperazin-1-yl)ethoxy)-1-methyl-1H-indazol-3-yl)piperidine-2,6-dione B38. ¹H NMR (400 MHz, DMSO-d₆) δ 10.88 (s, 1H), 8.19 (s, 1H), 7.57 (d, J=11.6 Hz, 1H), 7.17-7.11 (m, 4H), 6.85 (d, J=9.6 Hz, 2H), 6.75 (dd, J=2.0 Hz, 11.6 Hz, 1H), 6.68-6.63 (m, 2H), 6.56-6.50 (m, 3H), 6.28 (d, J=11.2 Hz, 2H), 6.32 (dd, J=6.8 Hz, 12.0 Hz, 1H), 4.18-4.16 (m, 3H), 3.95 (s, 3H), 3.84 (t, J=8.0 Hz, 2H), 3.35-3.31 (m, 1H), 2.99-2.93 (m, 2H), 2.76 (t, J=7.2 Hz, 2H), 2.68-2.62 (m, 2H), 2.62-2.51 (m, 4H), 2.52-2.46 (m, 4H), 2.42-2.41 (m, 2H), 2.35-2.30 (t, J=8.8 Hz, 2H), 2.21-2.02 (m, 2H), 1.75-1.64 (m, 2H), 1.55-1.51 (m, 2H); MS (ESI) m/z: 742.3 [M+H]⁺.

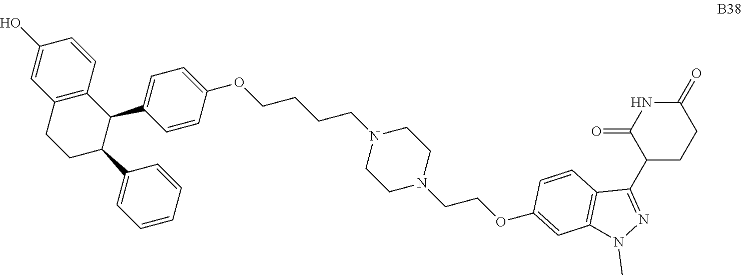 [SMILES: Cn1nc(C2CCC(=O)NC2=O)c2ccc(OCCN3CCN(CCCCOc4ccc([C@@H]5c6ccc(O)cc6CC[C@@H]5c5ccccc5)cc4)CC3)cc21]

3-(6-(2-(4-(4-((1R,2S)-6-Hydroxy-2-phenyl-1,2,3,4-tetrahydronaphthalen-1-yl)-phenyl)piperazin-1-yl)-2-oxoethoxy)-1-methyl-1H-indazol-3-yl)piperidine-2,6-dione B39. ¹H NMR (400 MHz, DMSO-d₆) δ 10.86 (s, 1H), 9.11 (s, 1H), 7.58 (d, J=7.6 Hz, 1H), 7.18-7.10 (m, 3H), 7.06 (d, J=2.0 Hz, 1H), 6.88-6.82 (m, 2H), 6.78 (dd, J=2.0 Hz, 8.8 Hz, 1H), 6.64 (d, J=8.4 Hz, 1H), 6.62-6.53 (m, 3H), 6.48 (dd, J=2.0 Hz, 8.4 Hz, 1H), 6.24 (d, J=8.8 Hz, 2H), 4.90 (s, 2H), 4.30 (dd, J=5.2 Hz, 9.6 Hz, 1H), 4.15 (d, J=4.8 Hz, 1H), 3.90 (s, 3H), 3.57-3.56 (m, 4H), 3.07-2.98 (m, 6H), 2.67-2.51 (m, 3H), 2.32-2.27 (m, 1H), 2.13-2.01 (m, 2H), 1.73-1.69 (m, 1H); MS (ESI) m/z: 684.1 [M+H]⁺.

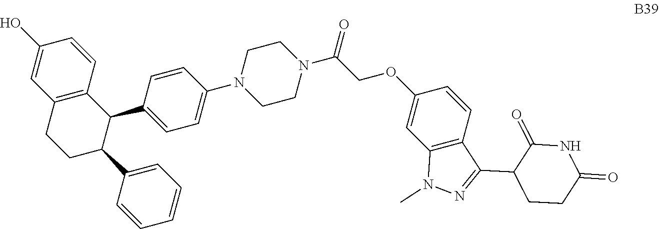 [SMILES: Cn1nc(C2CCC(=O)NC2=O)c2ccc(OCC(=O)N3CCN(c4ccc([C@@H]5c6ccc(O)cc6CC[C@@H]5c5ccccc5)cc4)CC3)cc21]

3-(6-(2-(4-((4-(4-((1R,2S)-6-Hydroxy-2-phenyl-1,2,3,4-tetrahydronaphthalen-1-yl)phenyl)piperazin-1-yl)methyl)piperidin-1-yl)-2-oxoethoxy)-1-methyl-1H-indazol-3-yl)piperidine-2,6-dione B40. ¹H NMR (400 MHz, DMSO-d₆) δ 10.87 (s, 1H), 9.10 (s, 1H), 8.13 (s. 1H), 7.57 (d, J=8.8 Hz, 1H), 7.16-7.10 (m, 3H), 7.04 (d, J=2.0 Hz, 1H), 6.83 (d, J=6.8 Hz, 2H), 6.77 (dd, J=2.0 Hz, 8.8 Hz, 1H), 6.64 (d, J=8.4 Hz, 1H), 6.60 (d, J=2.0 Hz, 1H), 6.53 (d, J=8.8 Hz, 2H), 6.48 (dd, J=2.0 Hz, 8.4 Hz, 1H), 6.22 (d, J=8.8 Hz, 2H), 4.85-4.84 (m, 2H), 4.32-4.28 (m, 2H), 4.14 (d, J=4.8 Hz, 1H), 3.91 (s, 3H), 3.90-3.84 (m, 1H), 3.33-3.30 (m, 4H), 3.05-2.92 (m, 7H), 2.67-2.52 (m, 3H), 2.33-2.28 (m, 2H), 2.19-2.07 (m, 4H), 1.81-1.72 (m, 4H), 1.14-1.11 (m, 1H), 0.96-0.93 (m, 1H); MS (ESI) m/z: 781.3 [M+H]⁺.

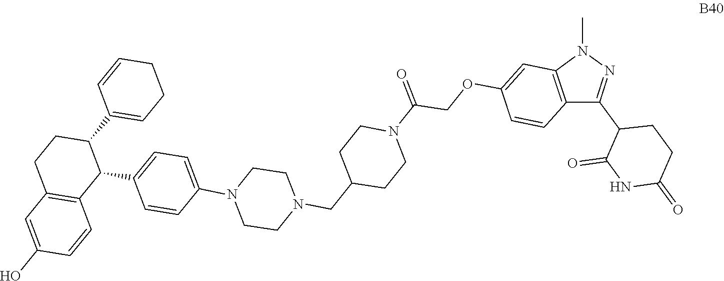 [SMILES: Cn1nc(C2CCC(=O)NC2=O)c2ccc(OCC(=O)N3CCC(CN4CCN(c5ccc([C@@H]6c7ccc(O)cc7CC[C@@H]6C6=CCCC=C6)cc5)CC4)CC3)cc21]

3-(6-(4-((4-(4-((1R,2S)-6-Hydroxy-2-phenyl-1,2,3,4-tetrahydronaphthalen-1-yl)-phenyl)piperidin-1-yl)methyl)piperidin-1-yl)-1-methyl-1H-indazol-3-yl)piperidine-2,6-dione B41. ¹H NMR (400 MHz, DMSO-d₆) δ 10.85 (s, 1H), 9.14 (brs, 1H), 8.17 (s, 1H), 7.47 (d, J=8.8 Hz, 1H), 7.16-7.08 (m, 3H), 6.89 (dd, J=1.6 Hz, 8.8 Hz, 1H), 6.86-6.79 (m, 5H), 6.66 (d, J=8.4 Hz, 1H), 6.61 (d, J=2.4 Hz, 1H), 6.48 (dd, J=2.4 Hz, 8.0 Hz, 1H), 6.29 (d, J=8.4 Hz, 2H), 4.24 (dd, J=5.2 Hz, 9.2 Hz, 1H), 4.20 (d, J=4.8 Hz, 1H), 3.88 (s, 3H), 3.77 (d, J=12.0 Hz, 2H), 2.99-2.90 (m, 4H), 2.71 (t, J=11.6 Hz, 2H), 2.64-2.58 (m, 2H), 2.38-2.22 (m, 2H), 2.20-1.87 (m, 6H), 1.80 (d, J=11.6 Hz, 2H), 1.75-1.41 (m, 5H), 1.34-1.12 (m, 4H); MS (ESI) m/z: 722.2 [M+H]⁺.

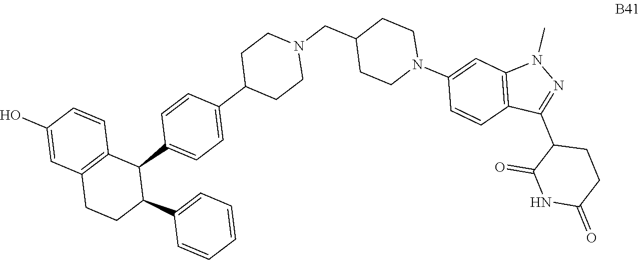 [SMILES: Cn1nc(C2CCC(=O)NC2=O)c2ccc(N3CCC(CN4CCC(c5ccc([C@@H]6c7ccc(O)cc7CC[C@@H]6c6ccccc6)cc5)CC4)CC3)cc21]

3-(6-(4-(3-(4-(4-((1R,2S)-6-Hydroxy-2-phenyl-1,2,3,4-tetrahydronaphthalen-1-yl)phenyl)piperidin-1-yl)propyl)piperidin-1-yl)-1-methyl-1H-indazol-3-yl)piperidine-2,6-dione B42. ¹H NMR (400 MHz, DMSO-d₆) δ 10.85 (s, 1H), 9.13 (brs, 1H), 8.18 (s, 1H), 7.46 (d, J=9.2 Hz, 1H), 7.13-7.10 (m, 3H), 6.89 (dd, J=1.2 Hz, 8.8 Hz, 1H), 6.85-6.75 (m, 5H), 6.67-6.60 (m, 2H), 6.48 (dd, J=2.4 Hz, 8.4 Hz, 1H), 6.29 (d, J=8.4 Hz, 2H), 4.24 (dd, J=5.2 Hz, 9.2 Hz, 1H), 4.19 (d, J=4.8 Hz, 1H), 3.88 (s, 3H), 3.77 (d, J=12.0 Hz, 2H), 3.04-2.85 (m, 4H), 2.74-2.64 (m, 2H), 2.64-2.57 (m, 2H), 2.38-2.22 (m, 4H), 2.20-2.04 (m, 2H), 2.03-1.91 (m, 3H), 1.80-1.60 (m, 5H), 1.58-1.35 (m, 5H), 1.32-1.23 (m, 4H); MS (ESI) m/z: 750.2 [M+H]⁺.

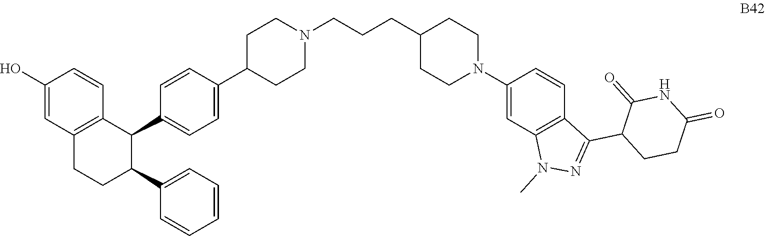 [SMILES: Cn1nc(C2CCC(=O)NC2=O)c2ccc(N3CCC(CCCN4CCC(c5ccc([C@@H]6c7ccc(O)cc7CC[C@@H]6c6ccccc6)cc5)CC4)CC3)cc21]

3-(6-(4-(2-(4-(4-((1R,2S)-6-Hydroxy-2-phenyl-1,2,3,4-tetrahydronaphthalen-1-yl)phenyl)piperidin-1-yl)ethyl)piperidin-1-yl)-1-methyl-1H-indazol-3-yl)piperidine-2,6-dione B43. ¹H NMR (400 MHz, DMSO-d₆) δ 10.83 (s, 1H), 9.11 (s, 1H), 7.46 (d, J=9.2 Hz, 1H), 7.14-7.10 (m, 3H), 6.90 (d, J=1.6 Hz, 1H), 6.88-6.78 (m, 5H), 6.66-6.61 (m, 2H), 6.48 (dd, J=2.4 Hz, 8.4 Hz, 1H), 6.29 (d, J=8.0 Hz, 2H), 4.24 (dd, J=5.2 Hz, 9.2 Hz, 1H), 4.19 (d, J=4.8 Hz, 1H), 3.88 (s, 3H), 3.75 (d, J=12.4 Hz, 2H), 2.99-2.92 (m, 4H), 2.69 (t, J=10.8 Hz, 2H), 2.65-2.57 (m, 2H), 2.40-2.30 (m, 4H), 2.25-2.05 (m, 2H), 2.00-1.90 (m, 2H), 1.90-1.40 (m, 11H), 1.35-1.25 (m, 2H); MS (ESI) m/z: 736.2 [M+H]⁺.

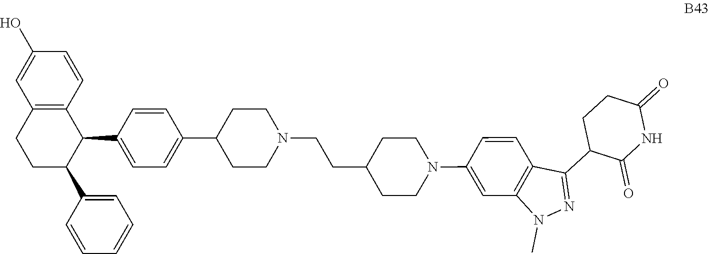 [SMILES: Cn1nc(C2CCC(=O)NC2=O)c2ccc(N3CCC(CCN4CCC(c5ccc([C@@H]6c7ccc(O)cc7CC[C@@H]6c6ccccc6)cc5)CC4)CC3)cc21]

3-(4-(2-(4-(4-(4-((1S,2R)-6-Hydroxy-2-phenyl-1,2,3,4-tetrahydronaphthalen-1-yl)phenoxy)butyl)piperazin-1-yl)-2-oxoethoxy)phenyl)piperidine-2,6-dione B44. ¹H NMR (400 MHz, DMSO-d₆) δ 10.79 (s, 1H), 9.11 (s, 1H), 7.17-7.10 (m, 5H), 6.86 (d, J=8.4 Hz, 2H), 6.82 (d, J=6.4 Hz, 2H), 6.64 (d, J=8.0 Hz, 2H), 6.61 (d, J=2.0 Hz, 1H), 6.52 (d, J=8.4 Hz, 2H), 6.47 (dd, J=2.4, 8.4 Hz, 1H), 6.25 (d, J=8.8 Hz, 2H), 4.78 (s, 2H), 4.17 (d, J=5.2 Hz, 1H), 3.83 (t, J=6.0 Hz, 2H), 3.79-3.76 (m, 1H), 3.42 (s, 4H), 3.28-3.25 (m, 1H), 2.98-2.96 (m, 2H), 2.65-2.61 (m, 1H), 2.46-2.31 (m, 7H), 2.17-2.11 (m, 2H), 2.02-1.99 (m, 1H), 1.68-1.63 (m, 3H), 1.54-1.53 (m, 2H); MS (ESI) m/z: 702.5 [M+H]⁺.

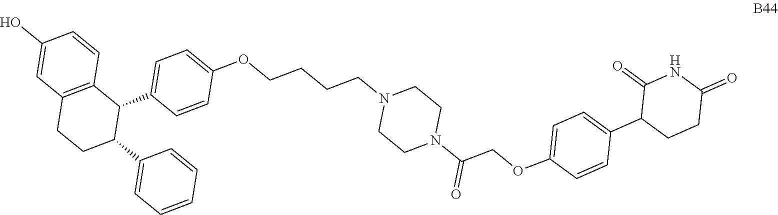 [SMILES: O=C1CCC(c2ccc(OCC(=O)N3CCN(CCCCOc4ccc([C@H]5c6ccc(O)cc6CC[C@H]5c5ccccc5)cc4)CC3)cc2)C(=O)N1]

1-(4-(2-(4-(4-(4-((1S,2R)-6-Hydroxy-2-phenyl-1,2,3,4-tetrahydronaphthalen-1-yl)phenoxy)butyl)piperazin-1-yl)-2-oxoethoxy)phenyl)dihydropyrimidine-2,4(1H,3H)-dione B45. ¹H NMR (400 MHz, DMSO-d₆) δ 10.28 (s, 1H), 9.10 (s, 1H), 7.21 (d, J=8.8 Hz, 2H), 7.17-7.10 (m, 3H), 6.91 (d, J=9.2 Hz, 2H), 6.82 (d, J=8.8 Hz, 2H), 6.63 (d, J=8.4 Hz, 1H), 6.61 (d, J=2.4 Hz, 1H), 6.52 (d, J=8.8 Hz, 2H), 6.47 (dd, J=2.4, 8.4 Hz, 1H), 6.26 (d, J=8.8 Hz, 2H), 4.80 (s, 2H), 4.17 (d, J=4.8 Hz, 1H), 3.82 (t, J=6.4 Hz, 2H), 3.70 (t, J=6.8 Hz, 2H), 3.42 (s, 4H), 3.29 (s, 1H), 3.03-2.91 (m, 2H), 2.68 (t, J=6.8 Hz, 2H), 3.36-2.30 (m, 5H), 2.10-1.99 (m, 1H), 1.71-1.52 (m, 6H); MS (ESI) m/z: 703.5 [M+H]⁺.

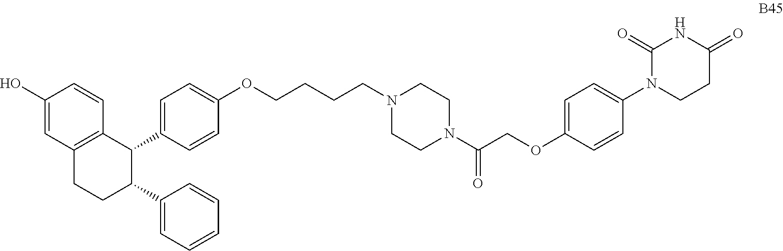 [SMILES: O=C1CCN(c2ccc(OCC(=O)N3CCN(CCCCOc4ccc([C@H]5c6ccc(O)cc6CC[C@H]5c5ccccc5)cc4)CC3)cc2)C(=O)N1]

3-(6-(2-(4-(4-(4-((1R,2S)-6-Hydroxy-2-phenyl-1,2,3,4-tetrahydronaphthalen-1-yl)phenoxy)butyl)piperazin-1-yl)-2-oxoethoxy)-1-methyl-1H-indol-3-yl)piperidine-2,6-dione B46. ¹H NMR (400 MHz, DMSO-d₆) δ 10.76 (s, 1H), 10.32 (s, 1H), 9.13 (s, 1H), 7.37 (d, J=8.8 Hz, 1H), 7.17-7.11 (m, 3H), 7.07 (s, 1H), 6.98 (d, J=2.0 Hz, 1H), 6.83 (d, J=6.4 Hz, 2H), 6.71 (dd, J=2.4, 7.2 Hz, 1H), 6.63 (d, J=8.4 Hz, 1H), 6.61 (d, J=2.4 Hz, 1H), 6.54 (d, J=8.8 Hz, 2H), 6.48 (dd, J=2.4, 8.0 Hz, 1H), 6.27 (d, J=8.8 Hz, 2H), 4.86 (d, J=3.6 Hz, 2H), 4.42 (d, J=12.8 Hz, 1H), 4.19 (d, J=4.8 Hz, 1H), 4.12-4.07 (m, 1H), 4.03 (dd, J=5.2, 10.0 Hz, 1H), 3.85 (t, J=5.6 Hz, 2H), 3.69 (s, 3H), 3.51 (d, J=9.2 Hz, 3H), 3.39-3.29 (m, 2H), 3.13-2.92 (m, 7H), 2.67-2.53 (m, 1H), 2.49-2.06 (m, 3H), 1.80-1.24 (m, 5H); MS (ESI) m/z: 755.2 [M+H]⁺.

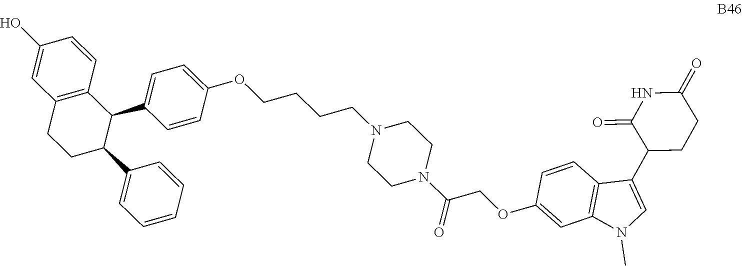 [SMILES: Cn1cc(C2CCC(=O)NC2=O)c2ccc(OCC(=O)N3CCN(CCCCOc4ccc([C@@H]5c6ccc(O)cc6CC[C@@H]5c5ccccc5)cc4)CC3)cc21]

3-(6-(2-(4-(4-(4-((1R,2S)-6-Hydroxy-2-phenyl-1,2,3,4-tetrahydronaphthalen-1-yl)phenoxy)butyl)piperazin-1-yl)-2-oxoethoxy)benzo[b]thiophen-3-yl)piperidine-2,6-dione B47. ¹H NMR (400 MHz, DMSO-d₆) δ 10.93 (s, 1H), 9.11 (s, 1H), 8.17 (s, 1H), 7.67 (d, J=8.8 Hz, 1H), 7.47 (s, 1H), 7.20 (s, 1H), 7.17-7.10 (m, 3H), 6.98 (dd, J=2.0, 8.4 Hz, 1H), 6.83 (d, J=7.2 Hz, 2H), 6.64 (d, J=8.0 Hz, 1H), 6.60 (d, J=2.0 Hz, 1H), 6.52 (d, J=8.4 Hz, 2H), 6.48 (dd, J=2.0, 8.0 Hz, 1H), 6.25 (d, J=8.4 Hz, 2H), 4.85 (s, 1H), 4.24 (dd, J=4.8, 10.0 Hz, 1H), 4.17 (d, J=5.2 Hz, 1H), 3.81 (t, J=6.0 Hz, 2H), 3.44 (s, 4H), 2.97-2.93 (m, 2H), 2.70-2.66 (m, 1H), 2.58-2.57 (m, 1H), 2.38 (s, 2H), 2.33-2.25 (m, 6H), 2.13-2.05 (m, 1H), 1.73-1.62 (m, 4H), 1.56-1.48 (m, 2H); MS (ESI) m/z: 758.1 [M+H]⁺.

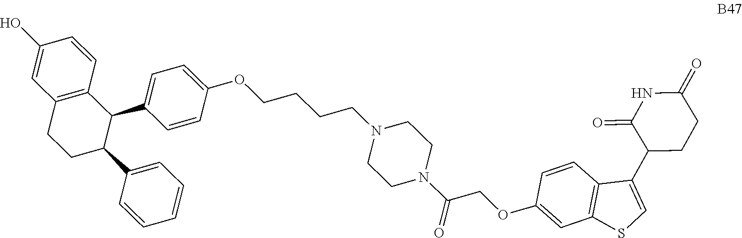 [SMILES: O=C1CCC(c2csc3cc(OCC(=O)N4CCN(CCCCOc5ccc([C@@H]6c7ccc(O)cc7CC[C@@H]6c6ccccc6)cc5)CC4)ccc23)C(=O)N1]

3-(5-(4-(4-(4-((5-Hydroxy-2-(4-hydroxyphenyl)-3-methyl-3H-indol-3-yl)-methyl)phenoxy)butyl)piperazin-1-yl)-1-oxoisoindolin-2-yl)piperidine-2,6-dione D2. ¹H NMR (400 MHz, DMSO-d₆) δ 10.93 (s, 1H), 9.96 (s, 1H), 9.33 (s, 1H), 8.03 (d, J=8.8 Hz, 2H), 7.51 (d, J=8.8 Hz, 1H), 7.12 (d, J=8.4 Hz, 1H), 7.06-7.02 (m, 2H), 6.90-6.86 (m, 3H), 6.63 (dd, J=2.0, 8.0 Hz, 1H), 6.44 (q, J=8.8 Hz, 4H), 5.04 (dd, J=4.8, 13.2 Hz, 1H), 4.34-4.18 (m, 2H), 3.78 (t, J=6.0 Hz, 2H), 3.44-3.41 (m, 2H), 3.27-3.19 (m, 8H), 2.94-2.84 (m, 1H), 2.61-2.50 (m, 1H), 2.39-2.32 (m, 3H), 2.03-1.93 (m, 1H), 1.67-1.58 (m, 2H), 1.57-1.48 (m, 5H); MS (ESI) m/z: 728.5 [M+H]⁺.

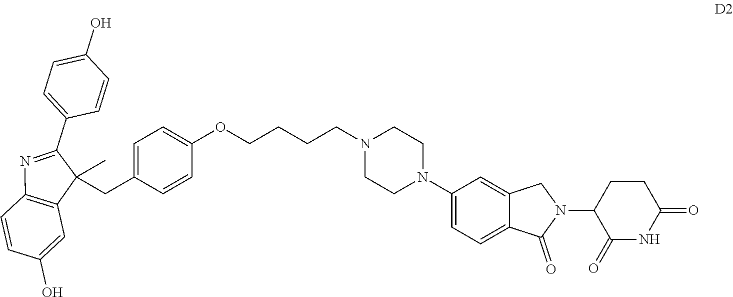 [SMILES: CC1(Cc2ccc(OCCCCN3CCN(c4ccc5c(c4)CN(C4CCC(=O)NC4=O)C5=O)CC3)cc2)C(c2ccc(O)cc2)=Nc2ccc(O)cc21]

3-(5-(2-(4-(4-(4-((5-Hydroxy-2-(4-hydroxyphenyl)-3-methyl-3H-indol-3-yl)-methyl)phenoxy)butyl)piperazin-1-yl)-2-oxoethoxy)-1-oxoisoindolin-2-yl)piperidine-2,6-dione D3. ¹H NMR (400 MHz, DMSO-d₆) δ 10.96 (s, 1H), 9.96 (s, 1H), 9.33 (s, 1H), 8.03 (d, J=8.4 Hz, 2H), 7.62 (d, J=8.4 Hz, 1H), 7.13-7.11 (m, 2H), 7.05-7.02 (m, 1H), 6.89-6.87 (m, 3H), 6.63 (dd, J=2.0, 8.0 Hz, 1H), 6.46-6.40 (m, 4H), 5.07 (dd, J=5.2, 13.2 Hz, 1H), 4.93 (s, 2H), 4.40-4.22 (m, 2H), 3.77 (t, J=6.8 Hz, 2H), 3.41-3.39 (m, 4H), 3.25-3.23 (m, 1H), 2.94-2.86 (m, 1H), 2.67-2.57 (m, 1H), 2.40-2.34 (m, 4H), 2.30-2.23 (m, 4H), 2.01-1.93 (m, 1H), 1.62-1.57 (m, 2H), 1.54 (s, 3H), 1.50-1.46 (m, 2H); MS (ESI) m/z: 786.5 [M+H]⁺.

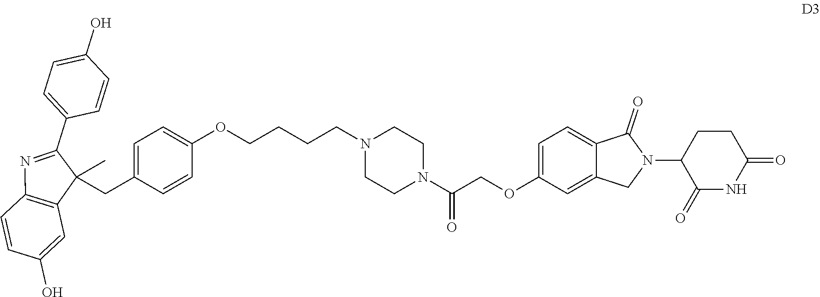 [SMILES: CC1(Cc2ccc(OCCCCN3CCN(C(=O)COc4ccc5c(c4)CN(C4CCC(=O)NC4=O)C5=O)CC3)cc2)C(c2ccc(O)cc2)=Nc2ccc(O)cc21]

3-(6-(2-(4-(4-(4-((5-Hydroxy-2-(4-hydroxyphenyl)-3-methyl-3H-indol-3-yl)-methyl)phenoxy)butyl)piperazin-1-yl)-2-oxoethoxy)-1-methyl-1H-indazol-3-yl)piperidine-2,6-dione D5. ¹H NMR (400 MHz, DMSO-d₆) δ 10.86 (s, 1H), 9.97 (s, 1H), 9.35 (s, 1H), 8.16 (s, 1H), 8.03 (d, J=8.8 Hz, 2H), 7.57 (d, J=8.8 Hz, 1H), 7.12 (d, J=8.0 Hz, 1H), 7.05-7.04 (m, 1H), 6.90-6.87 (m, 3H), 6.77 (dd, J=2.0, 8.8 Hz, 1H), 6.63 (dd, J=2.4, 8.0 Hz, 1H), 6.46-6.41 (m, 4H), 4.87 (s, 2H), 4.30 (dd, J=4.8, 9.2 Hz, 1H), 3.91 (s, 3H), 3.77 (t, J=6.4 Hz, 2H), 3.57-3.53 (m, 4H), 3.44-3.25 (m, 4H), 2.68-2.60 (m, 2H), 2.38-2.33 (m, 2H), 2.31-2.28 (m, 4H), 1.63-1.60 (m, 2H), 1.58 (s, 3H), 1.54-1.48 (m, 6H); MS (ESI) m/z: 785.5 [M+H]⁺.

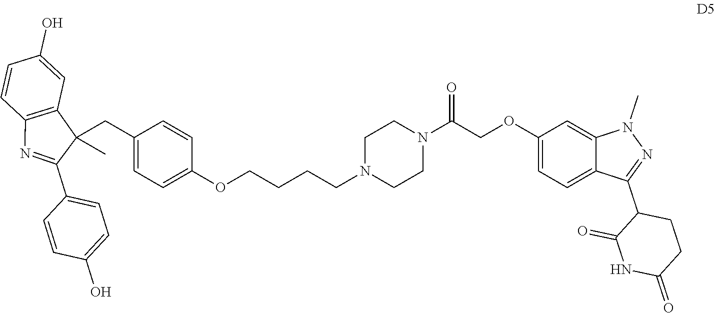 [SMILES: Cn1nc(C2CCC(=O)NC2=O)c2ccc(OCC(=O)N3CCN(CCCCOc4ccc(CC5(C)C(c6ccc(O)cc6)=Nc6ccc(O)cc65)cc4)CC3)cc21]

3-(4-(2-(4-(4-(4-((5-Hydroxy-2-(4-hydroxyphenyl)-3-methyl-3H-indol-3-yl)-methyl)phenoxy)butyl)piperazin-1-yl)-2-oxoethoxy)phenyl)piperidine-2,6-dione D6. ¹H NMR (400 MHz, DMSO-d₆) δ 10.78 (s, 1H), 9.94 (s, 1H), 9.32 (s, 1H), 8.03 (d, J=8.4 Hz, 2H), 7.11 (d, J=8.0 Hz, 3H), 6.89-6.85 (m, 5H), 6.63 (dd, J=2.0, 8.4 Hz, 1H), 6.48-6.43 (m, 4H), 4.77 (s, 2H), 3.78-3.76 (m, 3H), 3.41-3.39 (m, 2H), 3.25-3.24 (m, 1H), 2.68-2.61 (m, 2H), 2.35-2.33 (m, 2H), 2.29-2.26 (m, 4H), 2.16-2.11 (m, 1H), 2.09-2.08 (m, 2H), 1.90 (s, 2H), 1.62-1.57 (m, 2H), 1.54 (s, 3H), 1.51-1.46 (m, 2H); MS (ESI) m/z: 731.5 [M+H]⁺.

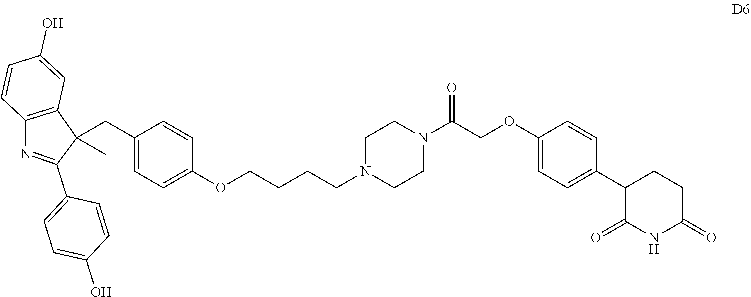 [SMILES: CC1(Cc2ccc(OCCCCN3CCN(C(=O)COc4ccc(C5CCC(=O)NC5=O)cc4)CC3)cc2)C(c2ccc(O)cc2)=Nc2ccc(O)cc21]

3-(3-(2-(4-(4-(4-((5-Hydroxy-2-(4-hydroxyphenyl)-3-methyl-3H-indol-3-yl)-methyl)phenoxy)butyl)piperazin-1-yl)-2-oxoethoxy)phenyl)piperidine-2,6-dione D7. ¹H NMR (400 MHz, DMSO-d₆) δ 10.80 (s, 1H), 9.98 (s, 1H), 9.34 (s, 1H), 8.02 (d, J=8.8 Hz, 2H), 7.19 (t, J=8.4 Hz, 1H), 7.08 (d, J=8.0 Hz, 1H), 6.85 (d, J=8.8 Hz, 3H), 6.79-6.76 (m, 3H), 6.59 (dd, J=2.0, 7.6 Hz, 1H), 6.43-6.38 (m, 4H), 4.74 (s, 2H), 3.76 (dd, J=4.4, 11.6 Hz, 1H), 3.72 (t, J=6.8 Hz, 2H), 3.41-3.40 (m, 2H), 3.12-3.04 (m, 1H), 2.65-2.58 (m, 1H), 2.43-2.40 (m, 1H), 2.31 (s, 2H), 2.26 (t, J=6.0 Hz, 4H), 2.20-2.12 (m, 2H), 2.02-1.95 (m, 3H), 1.61-1.54 (m, 2H), 1.51 (s, 3H), 1.47-1.42 (m, 2H); MS (ESI) m/z: 731.5 [M+H]⁺.

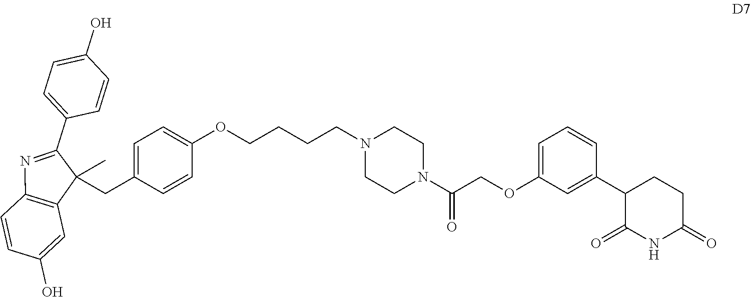 [SMILES: CC1(Cc2ccc(OCCCCN3CCN(C(=O)COc4cccc(C5CCC(=O)NC5=O)c4)CC3)cc2)C(c2ccc(O)cc2)=Nc2ccc(O)cc21]

1-(3-(2-(4-(4-(4-((5-Hydroxy-2-(4-hydroxyphenyl)-3-methyl-3H-indol-3-yl)-methyl)phenoxy)butyl)piperazin-1-yl)-2-oxoethoxy)phenyl)dihydropyrimidine-2,4(1H,3H)-dione D8. ¹H NMR (400 MHz, DMSO-d₆) δ 10.36 (s, 1H), 9.75 (brs, 2H), 8.09 (d, J=8.8 Hz, 2H), 7.28 (t, J=8.0 Hz, 1H), 7.15 (d, J=8.4 Hz, 1H), 6.98 (d, J=2.0 Hz, 1H), 6.96-6.92 (m, 4H), 6.82-6.80 (m, 1H), 6.69 (dd, J=2.0, 8.0 Hz, 1H), 6.48-6.40 (m, 4H), 4.87 (d, J=10.4 Hz, 2H), 4.43-4.32 (m, 1H), 4.02-4.01 (m, 1H), 3.81-3.75 (m, 6H), 3.52-3.48 (m, 4H), 3.36-3.33 (m, 2H), 3.17-3.00 (m, 2H), 2.70 (t, J=6.8 Hz, 2H), 1.73-1.64 (m, 4H), 1.61 (s, 3H); MS (ESI) m/z: 732.5 [M+H]⁺.

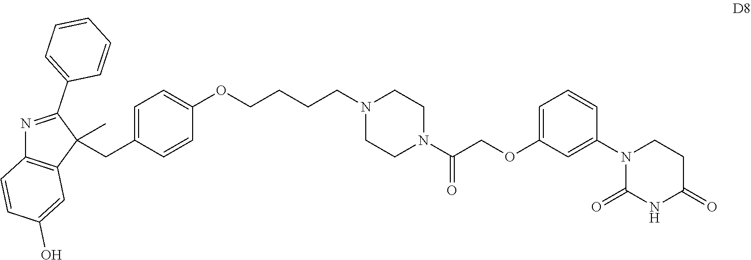 [SMILES: CC1(Cc2ccc(OCCCCN3CCN(C(=O)COc4cccc(N5CCC(=O)NC5=O)c4)CC3)cc2)C(c2ccccc2)=Nc2ccc(O)cc21]

1-(4-(2-(4-(4-(4-((5-Hydroxy-2-(4-hydroxyphenyl)-3-methyl-3H-indol-3-yl)-methyl)phenoxy)butyl)piperazin-1-yl)-2-oxoethoxy)phenyl)dihydropyrimidine-2,4(1H,3H)-dione D9. ¹H NMR (400 MHz, DMSO-d₆) δ 10.28 (s, 1H), 9.97 (s, 1H), 9.36 (s, 1H), 8.02 (d, J=8.8 Hz, 2H), 7.21 (d, J=8.8 Hz, 2H), 7.11 (d, J=8.4 Hz, 1H), 6.92-6.84 (m, 5H), 6.64-6.61 (m, 1H), 6.45-6.40 (m, 4H), 4.80 (s, 2H), 3.76 (t, J=6.0 Hz, 2H), 3.70 (t, J=6.8 Hz, 2H), 3.42-3.39 (m, 5H), 3.24 (s, 1H), 2.68 (t, J=6.8 Hz, 2H), 2.36-2.33 (m, 2H), 2.28 (t, J=6.8 Hz, 4H), 1.62-1.57 (m, 2H), 1.53 (s, 3H), 1.50-1.46 (m, 2H); MS (ESI) m/z: 732.3 [M+H]⁺.

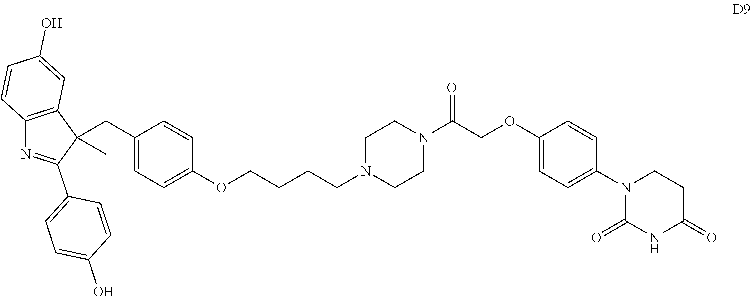 [SMILES: CC1(Cc2ccc(OCCCCN3CCN(C(=O)COc4ccc(N5CCC(=O)NC5=O)cc4)CC3)cc2)C(c2ccc(O)cc2)=Nc2ccc(O)cc21]

3-(5-(4-(4-(4-((5-Hydroxy-2-(4-hydroxyphenyl)-3-methyl-1H-indol-1-yl)methyl)-phenoxy)butyl)piperazin-1-yl)-1-oxoisoindolin-2-yl)piperidine-2,6-dione E2. ¹H NMR (400 MHz, DMSO-d₆) δ 10.93 (s, 1H), 9.64 (s, 1H), 8.67 (s, 1H), 7.51 (d, J=9.2 Hz, 1H), 7.15 (d, J=8.4 Hz, 2H), 7.07-7.02 (m, 3H), 6.85 (d, J=8.8 Hz, 2H), 6.80 (d, J=2.4 Hz, 1H), 6.75 (s, 4H), 6.57 (dd, J=2.4, 8.8 Hz, 1H), 5.10 (s, 2H), 5.04 (dd, J=5.2, 13.2 Hz, 1H), 4.34-4.18 (m, 2H), 3.89 (t, J=6.0 Hz, 2H), 3.28-3.23 (m, 6H), 2.95-2.84 (m, 1H), 2.61-2.52 (m, 1H), 2.38-2.31 (m, 3H), 2.10 (s, 3H), 2.03-1.94 (m, 3H), 1.72-1.64 (m, 2H), 1.60-1.52 (m, 2H); MS (ESI) m/z: 728.5 [M+H]⁺.

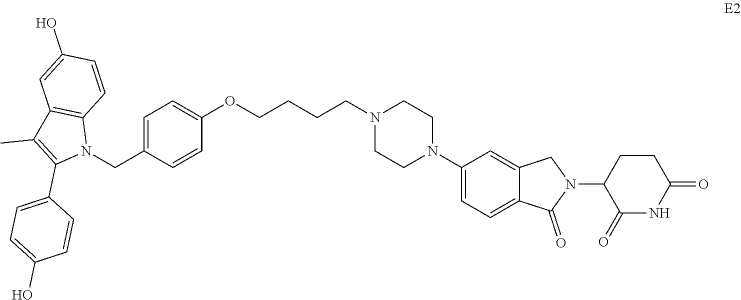 [SMILES: Cc1c(-c2ccc(O)cc2)n(Cc2ccc(OCCCCN3CCN(c4ccc5c(c4)CN(C4CCC(=O)NC4=O)C5=O)CC3)cc2)c2ccc(O)cc12]

3-(5-(2-(4-(4-(4-((5-Hydroxy-2-(4-hydroxyphenyl)-3-methyl-1H-indol-1-yl)-methyl)phenoxy)butyl)piperazin-1-yl)-2-oxoethoxy)-1-oxoisoindolin-2-yl)piperidine-2,6-dione E3. ¹H NMR (400 MHz, DMSO-d₆) δ 10.96 (s, 1H), 9.68 (s, 1H), 8.69 (s, 1H), 7.62 (d, J=8.4 Hz, 1H), 7.16 (d, J=8.4 Hz, 1H), 7.12 (s, 1H), 7.07-7.02 (m, 2H), 6.86 (d, J=8.4 Hz, 2H), 6.80 (d, J=2.0 Hz, 1H), 6.77-6.72 (m, 4H), 6.58 (dd, J=2.0, 8.8 Hz, 1H), 5.10-5.05 (m, 3H), 4.93 (s, 2H), 4.40-4.23 (m, 2H), 3.88 (t, J=6.4 Hz, 2H), 3.43-3.24 (m, 4H), 2.95-2.86 (m, 1H), 2.65-2.61 (m, 1H), 2.43-2.38 (m, 3H), 2.35-2.30 (m, 4H), 2.10 (s, 3H), 1.99-1.97 (m, 1H), 1.68-1.63 (m, 2H), 1.56-1.51 (m, 2H); MS (ESI) m/z: 786.5 [M+H]⁺.

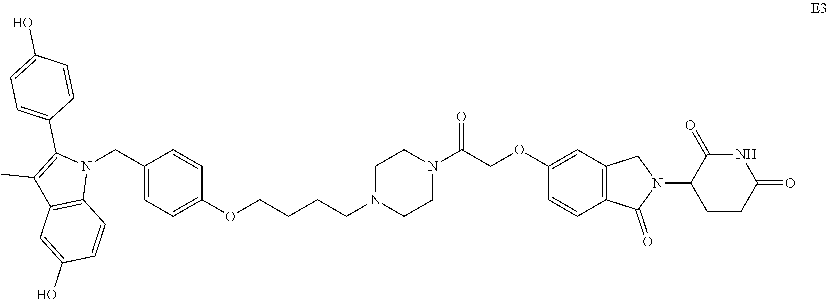 [SMILES: Cc1c(-c2ccc(O)cc2)n(Cc2ccc(OCCCCN3CCN(C(=O)COc4ccc5c(c4)CN(C4CCC(=O)NC4=O)C5=O)CC3)cc2)c2ccc(O)cc12]

3-(2-(4-(4-(4-((5-Hydroxy-2-(4-hydroxyphenyl)-3-methyl-1H-indol-1-yl)methyl)-phenoxy)butyl)piperazin-1-yl)-4-oxo-4,6-dihydro-5H-thieno[2,3-c]pyrrol-5-yl)piperidine-2,6-dione E6. ¹H NMR (400 MHz, DMSO-d₆) δ 10.82 (s, 1H), 9.56 (s, 1H), 8.59 (s, 1H), 7.07 (d, J=8.4 Hz, 2H), 6.97 (d, J=8.8 Hz, 1H), 6.76 (d, J=8.8 Hz, 2H), 6.70 (d, J=2.0 Hz, 1H), 6.68-6.64 (m, 4H), 6.47 (dd, J=2.4, 8.4 Hz, 1H), 6.19 (s, 1H), 5.00 (s, 2H), 4.87 (dd, J=5.2, 9.6 Hz, 1H), 4.31-4.15 (m, 2H), 3.79 (t, J=6.0 Hz, 2H), 3.40-3.34 (m, 4H), 3.09-3.04 (m, 4H), 2.80-2.72 (m, 1H), 2.57-2.45 (m, 3H), 2.24-2.20 (m, 1H), 2.00 (s, 3H), 1.91-1.85 (m, 1H), 1.58-1.50 (m, 4H); MS (ESI) m/z: 734.1 [M+H]⁺.

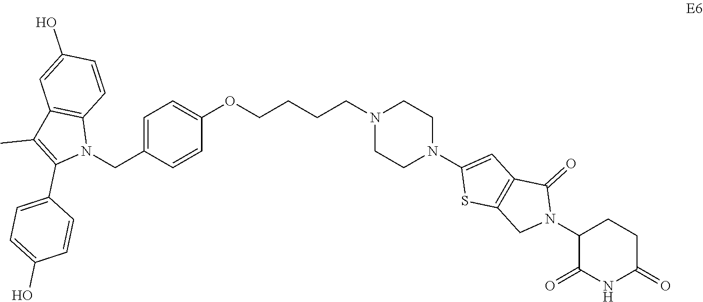 [SMILES: Cc1c(-c2ccc(O)cc2)n(Cc2ccc(OCCCCN3CCN(c4cc5c(s4)CN(C4CCC(=O)NC4=O)C5=O)CC3)cc2)c2ccc(O)cc12]

3-(7-(2-(4-(4-(4-((5-Hydroxy-2-(4-hydroxyphenyl)-3-methyl-1H-indol-1-yl)-methyl)phenoxy)butyl)piperazin-1-yl)-2-oxoethoxy)-1-methyl-1H-indazol-3-yl)piperidine-2,6-dione E7. ¹H NMR (400 MHz, DMSO-d₆) δ 10.87 (s, 1H), 9.65 (s, 1H), 8.68 (s, 1H), 7.23 (d, J=8.0 Hz, 1H), 7.16 (d, J=8.4 Hz, 2H), 7.06 (d, J=8.8 Hz, 1H), 6.96 (t, J=7.6 Hz, 1H), 6.85 (d, J=8.4 Hz, 2H), 6.82 (d, J=7.6 Hz, 1H), 6.80 (d, J=2.0 Hz, 1H), 6.77-6.72 (m, 4H), 6.57 (dd, J=2.4, 8.8 Hz, 1H), 5.10 (s, 2H), 4.99 (s, 2H), 4.33 (dd, J=5.2, 10.0 Hz, 1H), 4.23 (s, 3H), 3.88 (t, J=6.4 Hz, 2H), 3.46 (s, 4H), 2.67-2.60 (m, 2H), 2.38-2.30 (m, 7H), 2.19-2.14 (m, 1H), 2.10 (s, 3H), 1.69-1.65 (m, 2H), 1.55-1.51 (m, 2H); MS (ESI) m/z: 785.5 [M+H]⁺.

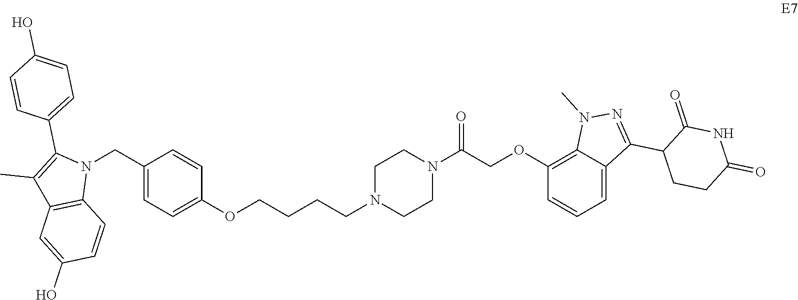 [SMILES: Cc1c(-c2ccc(O)cc2)n(Cc2ccc(OCCCCN3CCN(C(=O)COc4cccc5c(C6CCC(=O)NC6=O)nn(C)c45)CC3)cc2)c2ccc(O)cc12]

3-(7-((2-(4-(4-(4-((5-Hydroxy-2-(4-hydroxyphenyl)-3-methyl-1H-indol-1-yl)-methyl)phenoxy)butyl)piperazin-1-yl)-2-oxoethyl)amino)-1-methyl-1H-indazol-3-yl)piperidine-2,6-dione E8. ¹H NMR (400 MHz, DMSO-d₆) δ 10.84 (s, 1H), 9.66 (brs, 1H), 8.68 (brs, 1H), 8.15 (s, 1H), 7.15 (d, J=8.4 Hz, 2H), 7.06 (d, J=8.4 Hz, 1H), 6.95 (d, J=7.6 Hz, 1H), 6.89 (d, J=7.6 Hz, 1H), 6.85 (d, J=8.4 Hz, 2H), 6.80 (d, J=2.4 Hz, 1H), 6.78-6.70 (m, 4H), 6.57 (dd, J=2.4 Hz, 8.8 Hz, 1H), 6.42 (d, J=7.6 Hz, 1H), 5.71 (t, J=4.4 Hz, 1H), 5.10 (s, 2H), 4.28 (s, 3H), 4.26 (dd, J=5.2 Hz, 9.2 Hz, 1H), 3.98 (d, J=4.4 Hz, 2H), 3.88 (t, J=6.4 Hz, 2H), 3.56-3.47 (s, 4H), 2.65-2.59 (m, 2H), 2.40 (t, J=4.0 Hz, 2H), 2.34-2.24 (m, 5H), 2.16-2.12 (m, 1H), 2.08 (s, 3H), 1.70-1.64 (m, 2H), 1.57-1.50 (m, 2H); MS (ESI) m/z: 782.1 [M+H]⁺.

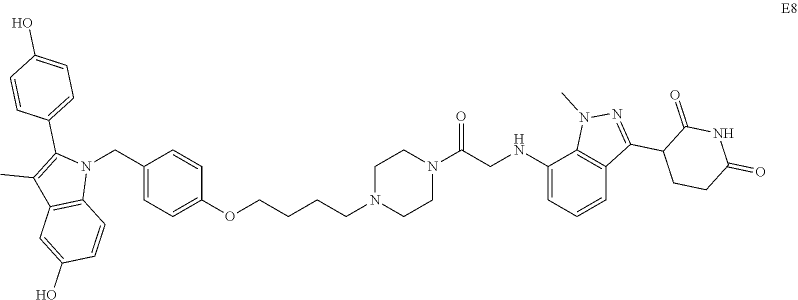 [SMILES: Cc1c(-c2ccc(O)cc2)n(Cc2ccc(OCCCCN3CCN(C(=O)CNc4cccc5c(C6CCC(=O)NC6=O)nn(C)c45)CC3)cc2)c2ccc(O)cc12]

3-(7-(2-(4-(4-(4-((5-Hydroxy-3-methyl-2-(4-(trifluoromethyl)phenyl)-1H-indol-1-yl)methyl)phenoxy)butyl)piperazin-1-yl)-2-oxoethoxy)-1-methyl-1H-indazol-3-yl)piperidine-2,6-dione E10. ¹H NMR (400 MHz, DMSO-d₆) δ 10.87 (s, 1H), 8.81 (brs, 1H), 7.84 (d, J=8.4 Hz, 2H), 7.60 (d, J=8.0 Hz, 2H), 7.23 (d, J=9.2 Hz, 1H), 7.18 (d, J=8.8 Hz, 1H), 6.96 (t, v=8.0 Hz, 1H), 6.86 (d, J=2.0 Hz, 1H), 6.82 (d, J=7.6 Hz, 1H), 6.72 (s, 4H), 6.66 (dd, J=2.0 Hz, 8.4 Hz, 1H), 5.17 (s, 2H), 4.99 (s, 2H), 4.32 (dd, J=4.8 Hz, 9.6 Hz, 1H), 4.22 (s, 3H), 3.87 (t, J=6.4 Hz, 2H), 3.49-3.41 (m, 4H), 2.73-2.56 (m, 2H), 2.40-2.36 (m, 2H), 2.33-2.29 (m, 4H), 2.19-2.13 (m, 4H), 2.03-1.96 (m, 1H), 1.70-1.62 (m, 2H), 1.57-1.49 (m, 2H); MS (ESI) m/z: 837.1 [M+H]⁺.

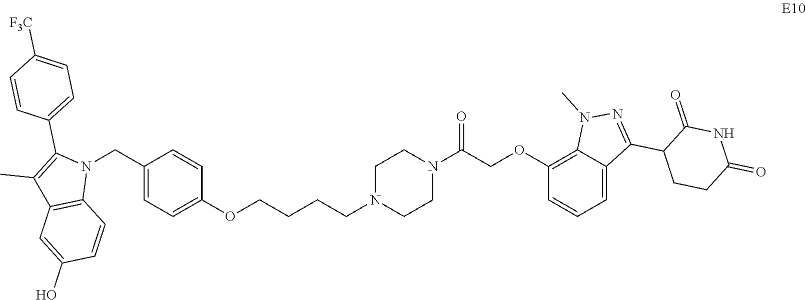 [SMILES: Cc1c(-c2ccc(C(F)(F)F)cc2)n(Cc2ccc(OCCCCN3CCN(C(=O)COc4cccc5c(C6CCC(=O)NC6=O)nn(C)c45)CC3)cc2)c2ccc(O)cc12]

3-(7-(2-(4-(4-(4-((2-(4-(Difluoromethyl)phenyl)-5-hydroxy-3-methyl-1H-indol-1-yl)methyl)phenoxy)butyl)piperazin-1-yl)-2-oxoethoxy)-1-methyl-1H-indazol-3-yl)piperidine-2,6-dione E11. ¹H NMR (400 MHz, DMSO-d₆) δ 10.88 (s, 1H), 8.78 (s, 1H), 7.68 (d, J=8.0 Hz, 2H), 7.52 (d, J=7.6 Hz, 2H), 7.23 (d, J=8.0 Hz, 1H), 7.15 (d, J=8.8 Hz, 1H), 7.10 (t, J=55.6 Hz, 1H), 6.96 (t, J=7.6 Hz, 1H), 6.85 (d, J=2.0 Hz, 1H), 6.82 (d, J=7.6 Hz, 1H), 6.73 (s, 4H), 6.64 (dd, J=2.0 Hz, 8.8 Hz, 1H), 5.16 (s, 2H), 5.00 (s, 2H), 4.31 (dd, J=5.2 Hz, 10.0 Hz, 1H), 4.22 (s, 3H), 3.87 (t, J=6.4 Hz, 2H), 3.50-3.40 (m, 4H), 2.67-2.60 (m, 2H), 2.38-2.31 (m, 7H), 2.18-2.17 (m, 1H), 2.15 (s, 3H), 1.68-1.63 (m, 2H), 1.54-1.50 (m, 2H); MS (ESI) m/z: 819.1 [M+H]⁺.

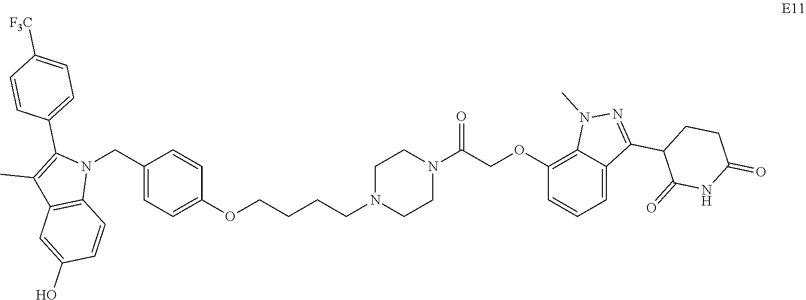 [SMILES: Cc1c(-c2ccc(C(F)(F)F)cc2)n(Cc2ccc(OCCCCN3CCN(C(=O)COc4cccc5c(C6CCC(=O)NC6=O)nn(C)c45)CC3)cc2)c2ccc(O)cc12]

3-(7-(2-(4-(4-((5-Hydroxy-2-(4-hydroxyphenyl)-3-methyl-1H-indol-1-yl)methyl)-phenyl)piperazin-1-yl)-2-oxoethoxy)-1-methyl-1H-indazol-3-yl)piperidine-2,6-dione E12. ¹H NMR (400 MHz, DMSO-d₆) δ 10.87 (s, 1H), 9.64 (s, 1H), 8.66 (s, 1H), 7.23 (d, J=8.4 Hz, 1H), 7.16 (d, J=8.4 Hz, 2H), 7.06 (d, J=8.8 Hz, 1H), 6.96 (t, J=8.0 Hz, 1H), 6.86-6.83 (m, 3H), 6.79-6.78 (m, 3H), 6.75-6.70 (m, 2H), 6.57 (dd, J=2.4 Hz, 8.4 Hz, 1H), 5.08 (s, 2H), 5.04 (s, 2H), 4.32 (dd, J=5.2 Hz, 9.6 Hz, 1H), 4.22 (s, 3H), 3.60-3.55 (m, 4H), 3.11-3.03 (m, 4H), 2.66-2.61 (m, 1H), 2.35-2.30 (m, 1H), 2.18-2.13 (m, 1H), 2.10 (s, 3H), 2.02-1.95 (m, 1H); MS (ESI) m/z: 713.3 [M+H]⁺.

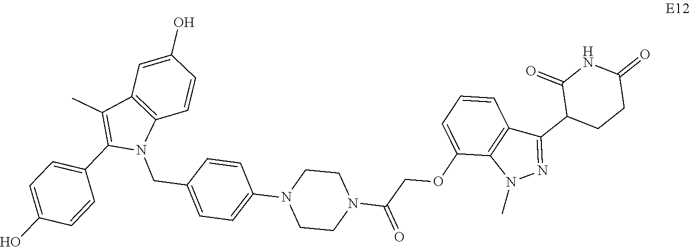 [SMILES: Cc1c(-c2ccc(O)cc2)n(Cc2ccc(N3CCN(C(=O)COc4cccc5c(C6CCC(=O)NC6=O)nn(C)c45)CC3)cc2)c2ccc(O)cc12]

3-(7-(2-(4-(4-(4-((5-Hydroxy-2-(4-hydroxyphenyl)-3-methyl-1H-indol-1-yl)-methyl)phenoxy)butyl)piperazin-1-yl)ethoxy)-1-methyl-1H-indazol-3-yl)piperidine-2,6-dione E13. ¹H NMR (400 MHz, DMSO-d₆) δ 10.86 (s, 1H), 9.86 (brs, 1H), 8.67 (brs, 1H), 8.16 (s, 1H), 7.21 (d, J=8.4 Hz, 1H), 7.15 (d, J=8.4 Hz, 2H), 7.05 (d, J=8.8 Hz, 1H), 6.97 (t, J=8.0 Hz, 1H), 6.85-6.83 (m, 3H), 6.79 (d, J=2.0 Hz, 1H), 6.78-6.68 (m, 4H), 6.57 (dd, J=2.0 Hz, 8.4 Hz, 1H), 5.09 (s, 2H), 4.31 (dd, J=5.2 Hz, 9.6 Hz, 1H), 4.21 (t, J=5.2 Hz, 2H), 4.17 (s, 3H), 3.86 (t, J=6.4 Hz, 2H), 3.64-3.47 (m, 2H), 3.13-3.06 (m, 1H), 2.78 (t, J=5.2 Hz, 2H), 2.66-2.55 (m, 3H), 2.36-2.55 (m, 7H), 2.17-2.13 (m, 1H), 2.09 (s, 3H), 1.66-1.62 (m, 2H), 1.51-1.47 (m, 2H); MS (ESI) m/z: 771.2 [M+H]⁺.

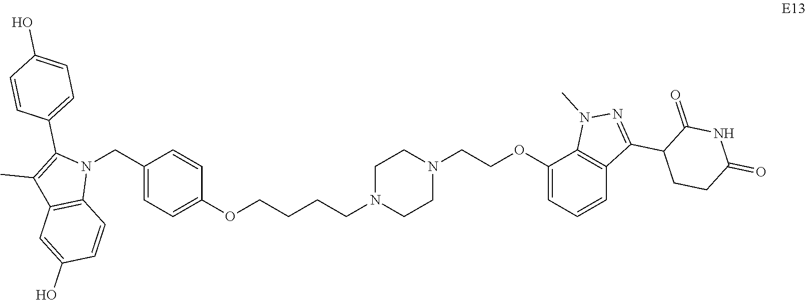 [SMILES: Cc1c(-c2ccc(O)cc2)n(Cc2ccc(OCCCCN3CCN(CCOc4cccc5c(C6CCC(=O)NC6=O)nn(C)c45)CC3)cc2)c2ccc(O)cc12]

3-(6-(2-(4-(4-(4-((5-Hydroxy-2-(4-hydroxyphenyl)-3-methyl-1H-indol-1-yl)-methyl)phenoxy)butyl)piperazin-1-yl)-2-oxoethoxy)-1-methyl-1H-indazol-3-yl)piperidine-2,6-dione E14. ¹H NMR (400 MHz, DMSO-d₆) δ 10.87 (s, 1H), 9.67 (s, 1H), 8.68 (s, 1H), 8.17 (s, 1H), 7.57 (d, J=8.8 Hz, 1H), 7.16 (d, J=8.8 Hz, 2H), 7.06 (d, J=12.8 Hz, 2H), 6.85 (d, J=8.8 Hz, 2H), 6.80-6.75 (m, 6H), 6.57 (dd, J=2.4, 8.8 Hz, 1H), 5.10 (s, 2H), 4.86 (s, 2H), 4.30 (dd, J=5.2, 9.6 Hz, 1H), 3.91 (s, 3H), 3.89-3.86 (m, 2H), 3.56-3.46 (m, 4H), 2.67-2.60 (m, 2H), 2.51-2.50 (m, 2H), 2.40-2.37 (m, 5H), 2.33-2.30 (m, 1H), 2.13 (s, 3H), 1.70-1.64 (m, 2H), 1.57-1.50 (m, 2H); MS (ESI) m/z: 785.4 [M+H]⁺.

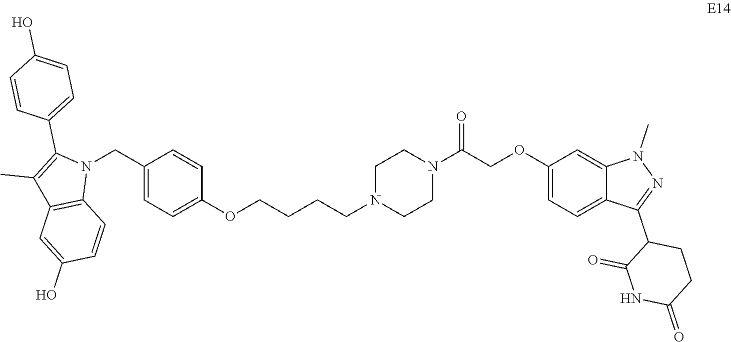 [SMILES: Cc1c(-c2ccc(O)cc2)n(Cc2ccc(OCCCCN3CCN(C(=O)COc4ccc5c(C6CCC(=O)NC6=O)nn(C)c5c4)CC3)cc2)c2ccc(O)cc12]

1-(6-(2-(4-(4-(4-((5-Hydroxy-2-(4-hydroxyphenyl)-3-methyl-1H-indol-1-yl)-methyl)phenoxy)butyl)piperazin-1-yl)-2-oxoethoxy)-1-methyl-1H-indazol-3-yl)dihydro-pyrimidine-2,4(1H,3H)-dione E16. ¹H NMR (400 MHz, DMSO-d₆) δ 10.54 (s, 1H), 9.71 (s, 1H), 8.71 (s, 1H), 8.18 (s, 1H), 7.52 (d, J=9.2 Hz, 1H), 7.16 (d, J=8.8 Hz, 2H), 7.07-7.04 (m, 2H), 6.85 (d, J=8.8 Hz, 2H), 6.85-6.74 (m, 6H), 6.57 (dd, J=2.4, 8.4 Hz, 1H), 5.10 (s, 2H), 4.87 (s, 2H), 3.91-3.86 (m, 7H), 3.46 (d, J=2.0 Hz, 4H), 2.74 (t, J=6.4 Hz, 2H), 2.40-2.30 (m, 6H), 2.10 (s, 3H), 1.69-1.65 (m, 2H), 1.55-1.51 (m, 2H); MS (ESI) m/z: 786.5 [M+H]⁺.

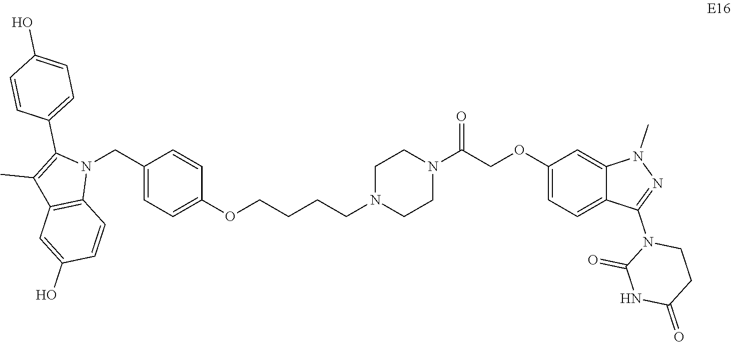 [SMILES: Cc1c(-c2ccc(O)cc2)n(Cc2ccc(OCCCCN3CCN(C(=O)COc4ccc5c(N6CCC(=O)NC6=O)nn(C)c5c4)CC3)cc2)c2ccc(O)cc12]

3-(6-(4-((1-(4-((5-Hydroxy-2-(4-hydroxyphenyl)-3-methyl-1H-indol-1-yl)-methyl)phenyl)piperidin-4-yl)methyl)piperazin-1-yl)-1-methyl-1H-indazol-3-yl)piperidine-2,6-dione E17. ¹H NMR (400 MHz, DMSO-d₆) δ 10.84 (s, 1H), 8.18 (s, 1H), 7.49 (d, J=9.2 Hz, 1H), 7.17 (d, J=8.4 Hz, 2H), 7.06 (d, J=8.8 Hz, 1H), 6.92-6.79 (m, 5H), 6.72 (q, J=8.8 Hz, 4H), 6.57 (dd, J=2.4, 8.8 Hz, 1H), 5.06 (s, 2H), 4.25 (dd, J=5.2, 9.2 Hz, 1H), 3.88 (s, 3H), 3.61-3.53 (m, 4H), 3.24-3.17 (m, 4H), 2.65-2.53 (m, 5H), 2.35-2.14 (m, 4H), 2.10 (s, 3H), 2.04-1.93 (m, 1H), 1.76 (d, J=12.0 Hz, 2H), 1.69-1.60 (m, 1H), 1.21-1.10 (m, 2H); MS (ESI) m/z: 752.3 [M+H]⁺.

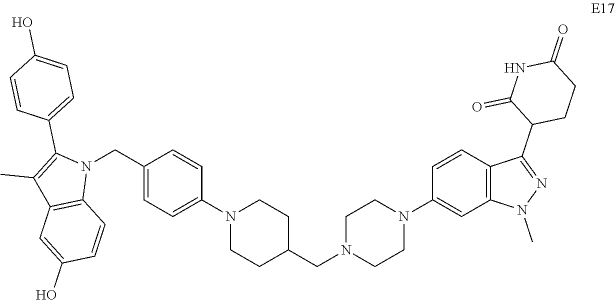 [SMILES: Cc1c(-c2ccc(O)cc2)n(Cc2ccc(N3CCC(CN4CCN(c5ccc6c(C7CCC(=O)NC7=O)nn(C)c6c5)CC4)CC3)cc2)c2ccc(O)cc12]

3-(6-(4-(2-(1-(4-((5-Hydroxy-2-(4-hydroxyphenyl)-3-methyl-1H-indol-1-yl)-methyl)phenyl)piperidin-4-yl)ethyl)piperazin-1-yl)-1-methyl-1H-indazol-3-yl)piperidine-2,6-dione E18. ¹H NMR (400 MHz, DMSO-d₆) δ 10.83 (s, 1H), 9.66 (brs, 1H), 8.66 (brs, 1H), 8.16 (s, 1H), 7.49 (d, J=8.8 Hz, 1H), 7.17 (d, J=8.4 Hz, 2H), 7.06 (d, J=8.4 Hz, 1H), 6.90 (dd, J=2.0 Hz, 9.2 Hz, 1H), 6.88-6.84 (m, 2H), 6.82 (d, J=1.6 Hz, 1H), 6.79 (d, J=2.0 Hz, 1H), 6.71 (q, J=8.8 Hz, 4H), 6.57 (dd, J=2.4 Hz, 8.4 Hz, 1H), 5.06 (s, 2H), 4.25 (dd, J=5.2 Hz, 9.2 Hz, 1H), 3.88 (s, 3H), 3.56 (d, J=12.0 Hz, 2H), 3.41-3.34 (m, 4H), 3.24-3.17 (m, 4H), 2.65-2.53 (m, 5H), 2.35-2.23 (m, 3H), 2.20-2.11 (m, 1H), 2.10 (s, 3H), 2.04-1.93 (m, 1H), 1.72 (d, J=11.6 Hz, 2H), 1.49-1.39 (m, 3H); MS (ESI) m/z: 767.2 [M+H]⁺.

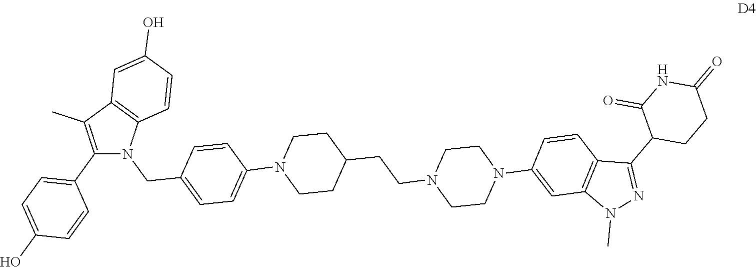 [SMILES: Cc1c(-c2ccc(O)cc2)n(Cc2ccc(N3CCC(CCN4CCN(c5ccc6c(C7CCC(=O)NC7=O)nn(C)c6c5)CC4)CC3)cc2)c2ccc(O)cc12]

3-(6-(2-(4-(3-(1-(4-((5-Hydroxy-2-(4-hydroxyphenyl)-3-methyl-1H-indol-1-yl)-methyl)phenyl)piperidin-4-yl)propyl)piperazin-1-yl)-2-oxoethoxy)-1-methyl-1H-indazol-3-yl)-piperidine-2,6-dione E19. ¹H NMR (400 MHz, DMSO-d₆) δ 10.86 (s, 1H), 9.65 (brs, 1H), 8.66 (brs, 1H), 8.16 (s, 1H), 7.57 (d, J=9.2 Hz, 1H), 7.16 (d, J=8.4 Hz, 2H), 7.08-7.03 (m, 2H), 6.85 (d, J=8.4 Hz, 2H), 6.81-6.75 (m, 2H), 6.71 (q, J=8.8 Hz, 4H), 6.57 (dd, J=2.4 Hz, 8.8 Hz, 1H), 5.05 (s, 2H), 4.86 (s, 2H), 4.30 (dd, J=5.2 Hz, 9.6 Hz, 1H), 3.91 (s, 3H), 3.56 (d, J=12.8 Hz, 2H), 3.51-3.43 (m, 4H), 2.68-2.54 (m, 2H), 2.43-2.38 (m, 2H), 2.37-2.24 (m, 6H), 2.20-2.12 (m, 1H), 2.10 (s, 3H), 2.04-1.95 (m, 1H), 1.69 (d, J=10.4 Hz, 2H), 1.50-1.41 (m, 3H), 1.35-1.29 (m, 2H), 1.22-1.10 (m, 2H); MS (ESI) m/z: 838.2 [M+H]⁺.

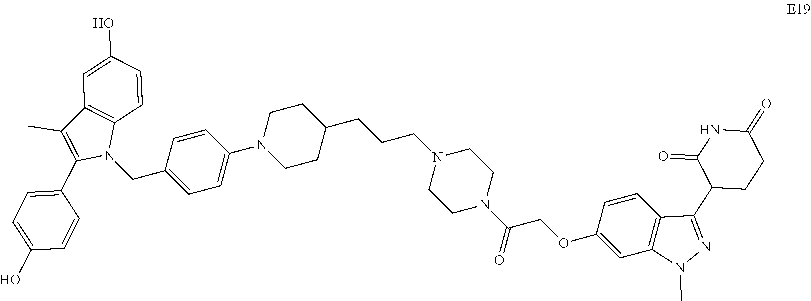 [SMILES: Cc1c(-c2ccc(O)cc2)n(Cc2ccc(N3CCC(CCCN4CCN(C(=O)COc5ccc6c(C7CCC(=O)NC7=O)nn(C)c6c5)CC4)CC3)cc2)c2ccc(O)cc12]

3-(6-(4-(3-(1-(4-((5-Hydroxy-2-(4-hydroxyphenyl)-3-methyl-1H-indol-1-yl)-methyl)phenyl)piperidin-4-yl)propyl)piperazin-1-yl)-1-methyl-1H-indazol-3-yl)piperidine-2,6-dione E20. ¹H NMR (400 MHz, DMSO-d₆) δ 10.84 (s, 1H), 9.68 (brs, 1H), 8.67 (brs, 1H), 8.16 (s, 1H), 7.49 (d, J=8.8 Hz, 1H), 7.17 (d, J=8.4 Hz, 2H), 7.06 (d, J=8.8 Hz, 1H), 6.92 (dd, J=2.0 Hz, 8.8 Hz, 1H), 6.88-6.81 (m, 3H), 6.79 (d, J=2.0 Hz, 1H), 6.71 (q, J=8.8 Hz, 4H), 6.57 (dd, J=2.4 Hz, 8.8 Hz, 1H), 5.06 (s, 2H), 4.25 (dd, J=5.2 Hz, 9.2 Hz, 1H), 3.88 (s, 3H), 3.60-3.53 (m, 4H), 3.24-3.17 (m, 4H), 2.68-2.53 (m, 7H), 2.37-2.24 (m, 3H), 2.21-2.12 (m, 1H), 2.10 (s, 3H), 1.70 (d, J=11.6 Hz, 2H), 1.55-1.44 (m, 1H), 1.39-1.30 (m, 2H), 1.21-1.10 (m, 2H); MS (ESI) m/z: 780.3 [M+H]⁺.

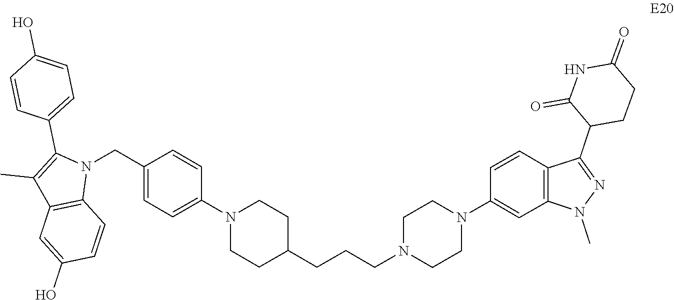 [SMILES: Cc1c(-c2ccc(O)cc2)n(Cc2ccc(N3CCC(CCCN4CCN(c5ccc6c(C7CCC(=O)NC7=O)nn(C)c6c5)CC4)CC3)cc2)c2ccc(O)cc12]

3-(6-(2-(4-(2-(1-(4-((5-Hydroxy-2-(4-hydroxyphenyl)-3-methyl-1H-indol-1-yl)-methyl)phenyl)piperidin-4-yl)ethyl)piperazin-1-yl)-2-oxoethoxy)-1-methyl-1H-indazol-3-yl)-piperidine-2,6-dione E21. ¹H NMR (400 MHz, DMSO-d₆) δ 11.10 (s, 1H), 10.86 (s, 1H), 9.73 (brs, 1H), 8.57 (brs, 1H), 7.59 (d, J=8.8 Hz, 1H), 7.17 (d, J=8.4 Hz, 2H), 7.09 (d, J=1.6 Hz, 1H), 7.06 (d, J=8.4 Hz, 1H), 6.87-6.79 (m, 6H), 6.58 (dd, J=2.4, 8.8 Hz, 1H), 5.33 (s, 2H), 4.96 (d, J=2.4 Hz, 2H), 4.41 (d, J=11.2 Hz, 1H), 4.31 (dd, J=5.2, 9.2 Hz, 1H), 4.08 (d, J=13.2 Hz, 1H), 3.93 (s, 3H), 3.57-3.50 (m, 4H), 3.24-3.08 (m, 6H), 2.99-2.92 (m, 2H), 2.71-2.59 (m, 2H), 2.33-2.30 (m, 1H), 2.21-2.14 (m, 1H), 2.11 (s, 3H), 2.01-1.96 (m, 1H), 1.90-1.82 (m, 2H), 1.77-1.57 (m, 4H); MS (ESI) m/z: 824.4 [M+H]⁺.

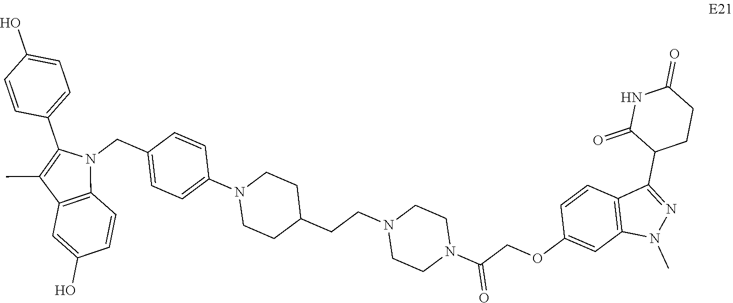 [SMILES: Cc1c(-c2ccc(O)cc2)n(Cc2ccc(N3CCC(CCN4CCN(C(=O)COc5ccc6c(C7CCC(=O)NC7=O)nn(C)c6c5)CC4)CC3)cc2)c2ccc(O)cc12]

3-(6-(2-(4-(4-(4-((5-Hydroxy-3-methyl-2-(4-(trifluoromethyl)phenyl)-1H-indol-1-yl)methyl)phenoxy)butyl)piperazin-1-yl)-2-oxoethoxy)-1-methyl-1H-indazol-3-yl)piperidine-2,6-dione E22. ¹H NMR (400 MHz, DMSO-d₆) δ 10.87 (s, 1H), 8.84 (brs, 1H), 7.84 (d, J=8.0 Hz, 1H), 7.64-7.55 (m, 3H), 7.18 (d, J=9.2 Hz, 1H), 7.04 (d, J=2.0 Hz, 1H), 6.87 (d, J=7.6 Hz, 1H), 6.77 (dd, J=2.4 Hz, 9.2 Hz, 1H), 6.76-6.69 (m, 4H), 6.66 (dd, J=2.4 Hz, 8.4 Hz, 1H), 5.17 (s, 2H), 4.86 (s, 2H), 4.30 (dd, J=5.2 Hz, 9.6 Hz, 1H), 3.91 (s, 3H), 3.87 (t, J=6.0 Hz, 2H), 3.49-3.41 (m, 4H), 2.73-2.56 (m, 2H), 2.43-2.36 (m, 2H), 2.35-2.27 (m, 5H), 2.20-2.12 (m, 4H), 1.70-1.62 (m, 2H), 1.57-1.49 (m, 2H); MS (ESI) m/z: 837.2 [M+H]⁺.

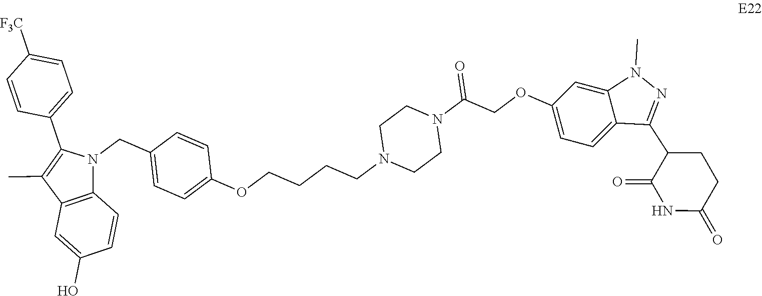 [SMILES: Cc1c(-c2ccc(C(F)(F)F)cc2)n(Cc2ccc(OCCCCN3CCN(C(=O)COc4ccc5c(C6CCC(=O)NC6=O)nn(C)c5c4)CC3)cc2)c2ccc(O)cc12]

1-(7-(2-(4-(4-(4-((5-Hydroxy-2-(4-hydroxyphenyl)-3-methyl-1H-indol-1-yl)-methyl)phenoxy)butyl)piperazin-1-yl)-2-oxoethoxy)-1-methyl-1H-indazol-3-yl)dihydro-pyrimidine-2,4(1H,3H)-dione E23. ¹H NMR (400 MHz, DMSO-d₆) δ 10.53 (s, 1H), 9.64 (s, 1H), 8.67 (s, 1H), 7.17-7.15 (m, 3H), 7.06 (d, J=8.8 Hz, 1H), 6.96 (t, J=8.0 Hz, 1H), 6.86-6.84 (m, 3H), 6.80 (d, J=2.4 Hz, 1H), 6.77-6.72 (m, 4H), 6.57 (dd, J=2.4, 8.8 Hz, 1H), 5.10 (s, 2H), 5.01 (s, 2H), 4.23 (s, 3H), 3.90-3.87 (m, 4H), 3.50-3.42 (m, 4H), 2.75 (t, J=6.8 Hz, 2H), 2.40-2.38 (m, 2H), 2.33-2.30 (m, 4H), 2.10 (s, 3H), 1.69-1.63 (m, 2H), 1.57-1.51 (m, 2H); MS (ESI) m/z: 786.5 [M+H]⁺.

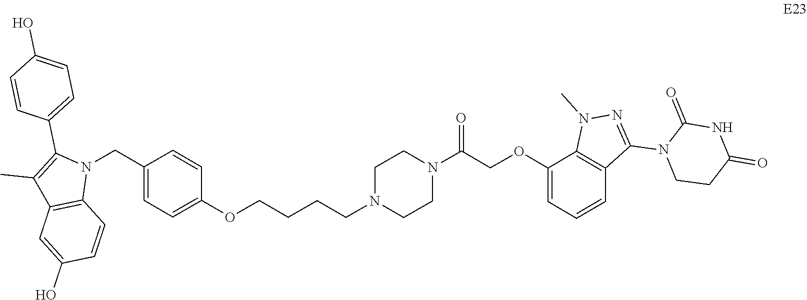 [SMILES: Cc1c(-c2ccc(O)cc2)n(Cc2ccc(OCCCCN3CCN(C(=O)COc4cccc5c(N6CCC(=O)NC6=O)nn(C)c45)CC3)cc2)c2ccc(O)cc12]

3-(3-(2-(4-(4-(4-((5-Hydroxy-2-(4-hydroxyphenyl)-3-methyl-1H-indol-1-yl)-methyl)phenoxy)butyl)piperazin-1-yl)-2-oxoethoxy)phenyl)piperidine-2,6-dione E25. ¹H NMR (400 MHz, DMSO-d₆) δ 10.82 (s, 1H), 9.66 (s, 1H), 8.67 (s, 1H), 8.14 (s, 1H), 7.22 (t, J=8.8 Hz, 1H), 7.15 (d, J=8.4 Hz, 1H), 6.84 (d, J=8.4 Hz, 2H), 6.80-6.76 (m, 4H), 6.74-6.72 (m, 4H), 6.56 (dd, J=2.0, 8.4 Hz, 1H), 5.10 (s, 2H), 4.76 (s, 2H), 3.88 (t, J=6.0 Hz, 2H), 3.81 (dd, J=5.2, 11.2 Hz, 1H), 3.43-3.3 (m, 4H), 2.69-2.60 (m, 2H), 2.36-2.29 (m, 6H), 2.20-2.14 (m, 1H), 2.10 (s, 3H), 2.06-2.00 (m, 1H), 1.68-1.63 (m, 2H), 1.56-1.49 (m, 2H); MS (ESI) m/z: 731.4 [M+H]⁺.

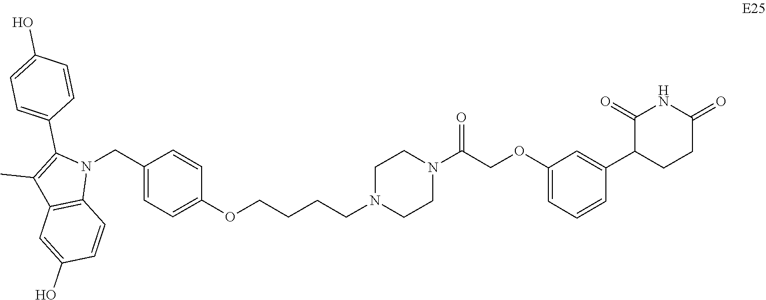

1-(3-(2-(4-(4-(4-((5-Hydroxy-2-(4-hydroxyphenyl)-3-methyl-1H-indol-1-yl)-methyl)phenoxy)butyl)piperazin-1-yl)-2-oxoethoxy)phenyl)dihydropyrimidine-2,4(1H,3H)-dione E26. ¹H NMR (400 MHz, DMSO-d₆) δ 10.36 (s, 1H), 10.15 (brs, 1H), 9.67 (s, 1H), 8.68 (s, 1H), 7.28 (t, J=8.0 Hz, 1H), 7.16 (d, J=8.4 Hz, 2H), 7.06 (d, J=8.4 Hz, 1H), 6.94-6.91 (m, 2H), 6.85 (t, J=8.0 Hz, 2H), 6.82-6.79 (m, 2H), 6.78-6.73 (m, 4H), 6.57 (dd, J=2.4, 8.8 Hz, 1H), 5.11 (s, 2H), 4.91-4.82 (m, 2H), 4.38 (d, J=11.2 Hz, 1H), 4.03-4.00 (m, 1H), 3.91 (t, J=5.6 Hz, 2H), 3.76 (t, J=6.8 Hz, 2H), 3.48 (d, J=11.2 Hz, 3H), 3.12-2.89 (m, 5H), 2.69 (t, J=6.8 Hz, 2H), 2.10 (s, 3H), 1.79-1.66 (m, 4H); MS (ESI) m/z: 732.5 [M+H]⁺.

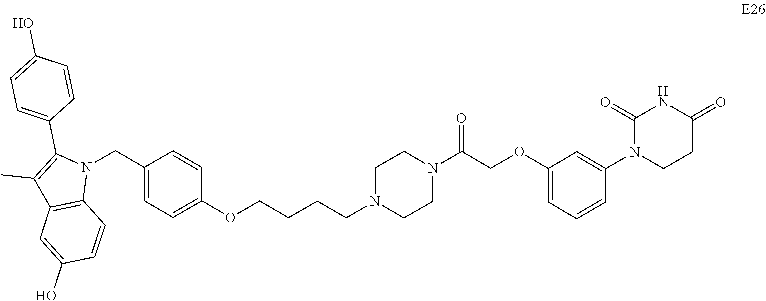

The following compounds are prepared similarly according to the synthetic procedures or methodologies exemplified herein.

3-(1-(1-(4-(4-((1R,2S)-6-Hydroxy-2-phenyl-1,2,3,4-tetrahydronaphthalen-1-yl)phenoxy)butyl)piperidin-4-yl)-4-oxo-4H-thieno[3,4-c]pyrrol-5(6H)-yl)piperidine-2,6-dione A8.

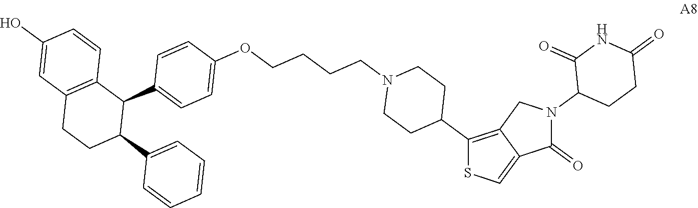

3-(2-(1-(4-(4-((1R,2S)-6-Hydroxy-2-phenyl-1,2,3,4-tetrahydronaphthalen-1-yl)phenoxy)butyl)piperidin-4-yl)-4-oxo-4,6-dihydro-5H-thieno[2,3-c]pyrrol-5-yl)piperidine-2,6-dione A11.

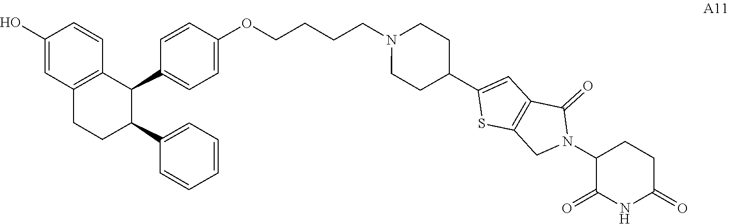

3-((4-((4-(((4-(4-((1S,2R)-6-Hydroxy-2-phenyl-1,2,3,4-tetrahydronaphthalen-1-yl)phenoxy)butyl)amino)methyl)benzyl)oxy)phenyl)amino)piperidine-2,6-dione B18.

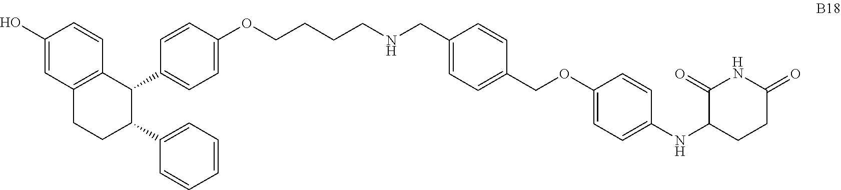

3-((3-((2-(4-((1-(4-((1R,2S)-6-Hydroxy-2-phenyl-1,2,3,4-tetrahydronaphthalen-1-yl)phenyl)piperidin-4-yl)methyl)piperazin-1-yl)-2-oxoethyl)(methyl)amino)phenyl)amino)-piperidine-2,6-dione B22.

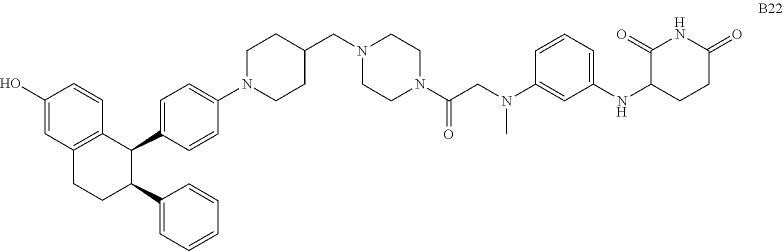

### Example B1

### Cell Viability Assay

A compound provided herein was evaluated for its anticancer activity in MCF7 and T47D cells. MCF7 and T47D cells were cultured at 37° C. in a humidified 5% C02 environment in MEM or RPMI, supplemented with 10% FBS with insulin, respectively. The cells were plated in 384-well microplates at a density of 1,000 cells per well in their respective maintenance media. The cells were treated with the compound by a 10-point dose response curve for 6 days, during which, the medium and compound were refreshed once. After the 6-day treatment, the cell viability was assessed using CELLTITER-GLO (CTG) reagent. Luminescence was measured with a microplate reader. The results are summarized in Table 1.

TABEL 1
Inhibition of Cancerous Cells

| | IC₅₀ (nM) | |
|---|---|---|
| Compound | MCF-7 | T47D |
| A1 | <0.01 | 0.24 |
| A2 | 0.72 | 0.61 |
| A3 | 0.2 | 0.44 |
| A4 | 0.06 | 0.08 |
| A5 | 0.37 | 0.22 |
| A6 | 4 | 0.47 |
| A7 | 145 | 0.18 |
| A9 | 1.4 | |
| A10 | 0.82 | |
| A14 | 1.1 | 0.86 |
| A15 | 1.2 | 0.96 |
| A16 | 0.68 | 0.22 |
| A17 | 1155 | |
| A18 | 0.28 | |
| A19 | 2.2 | 0.07 |
| A20 | 0.05 | |
| B2 | 0.25 | 0.01 |
| B5 | 1.2 | 0.48 |
| B6 | 0.22 | 0.18 |
| B9 | 1.7 | 0.49 |
| B10 | 1.6 | 0.76 |
| B13 | 16 | 4.6 |
| B14 | 0.73 | 1.2 |
| B16 | 5 | 2.6 |
| B19 | 2.5 | 0.96 |
| B20 | 5.7 | 0.93 |
| B23 | 0.22 | |
| B24 | 66 | |
| B25 | 1.2 | |
| B26 | 0.13 | |
| B27 | 0.99 | |
| B44 | 107 | |
| B45 | 88 | |
| D3 | 569 | |
| D4 | | 84 |
| D5 | 136 | |
| D7 | 101 | 44 |
| D15 | 12 | 4.2 |
| D23 | 1.7 | |
| E1 | 19 | 0.84 |
| E2 | 0.45 | 0.25 |
| E3 | 4.5 | 3.2 |
| E4 | 0.38 | 0.17 |
| E5 | 0.09 | 0.02 |
| E6 | 0.22 | |
| E7 | 1.1 | 0.64 |
| E14 | 0.33 | |
| E15 | 0.27 | |
| E16 | 1.8 | 1.3 |
| E17 | 0.8 | |
| E18 | 0.36 | |
| E24 | 1.7 | |
| E25 | 5 | |
| E26 | 3.8 | |
| E27 | 5.6 | 3.2 |

### Example B2

### Protein Degradation Assay

A compound provided herein was assessed for its activity in degrading an ERα in MCF-7 cells via a western blot. The MCF7 cells were cultured at 37° C. in a humidified 5% C02 environment in MEM supplemented with 10% FBS with insulin. The cells were seeded in 12-well plates at a density of 300,000 cells per well in maintenance media. Cells were treated with the compound at predetermined concentrations (e.g., 1 nM, 10 nM, 100 nM, and 1 μM) for 6 h. The cells were then washed in PBS and the whole cells were lysed in a cell lysis buffer and incubated for 15 minutes on ice. Cell debris was removed by high-speed centrifugation and the cleared whole cell lysates were prepared for further analysis by a western blot. The whole cell protein extracts were separated on SDS-polyacrylamide gels, transferred to nitrocellulose membranes, and probed with primary antibodies, including anti-ERα antibody and GAPDH antibody for loading control. The membranes were subsequently washed and probed with secondary antibodies for chemiluminescence, followed by imaging on a BIORAD CHEMIDOC Imaging System. The results are summarized in Table 2, where “A” represents a percentage degradation value of no less than 50%; “B” represents a percentage degradation value of less than 50% and no less than 25%; “C” represents a percentage degradation value of less than 25% and no less than 10%; and “D” represents a percentage degradation value of less than 10%.

TABEL 2
Effect on ERα Protein Degradation

| | Protein Degradation | | |
|---|---|---|---|
| Compound | 100 nM | 10 nM | 1 nM |
| A1 | B | C | C |
| A2 | D | D | D |
| A3 | D | D | D |
| A4 | D | D | D |
| A5 | A | A | A |
| A6 | D | B | B |
| A7 | D | D | D |
| A9 | B | B | B |
| A10 | D | D | D |
| A12 | C | C | C |
| A13 | C | B | C |
| A14 | A | A | A |
| A15 | D | C | D |
| A16 | A | A | B |
| A17 | D | C | B |
| A19 | D | D | D |
| A20 | D | D | D |
| B2 | C | B | B |
| B5 | D | D | D |
| B6 | C | C | C |
| B9 | D | D | D |
| B10 | D | D | D |
| B13 | D | D | D |
| B14 | D | D | D |
| B16 | C | C | C |
| B19 | D | D | D |
| B20 | D | C | B |
| B23 | D | B | B |
| B24 | C | C | C |
| B25 | D | B | C |
| B27 | D | B | D |
| B44 | C | C | B |
| B45 | C | C | C |
| D1 | D | D | D |
| D2 | D | D | D |
| D3 | D | D | D |
| D4 | C | D | D |
| D5 | C | D | C |
| D7 | D | D | D |
| D8 | D | D | D |
| D9 | D | D | D |
| D15 | D | D | C |
| D23 | D | C | C |
| E1 | B | B | C |
| E2 | D | D | D |
| E3 | B | B | B |
| E4 | C | B | B |
| E5 | D | D | D |
| E6 | D | D | D |
| E7 | A | A | B |
| E14 | D | B | B |
| E16 | D | C | C |
| E24 | D | D | D |
| E25 | D | D | D |
| E26 | D | D | D |
| E27 | D | D | D |

The examples set forth above are provided to give those of ordinary skill in the art with a complete disclosure and description of how to make and use the claimed embodiments and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed is:

1. A compound of Formula (I):
R^{T}-L-R^{E} (I)
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof,
wherein:
L is a linker;
R^{E} is an E3 ubiquitin ligase binding moiety; and
R^{T} is a moiety having the structure of any one of Formulae (i) to (xv): 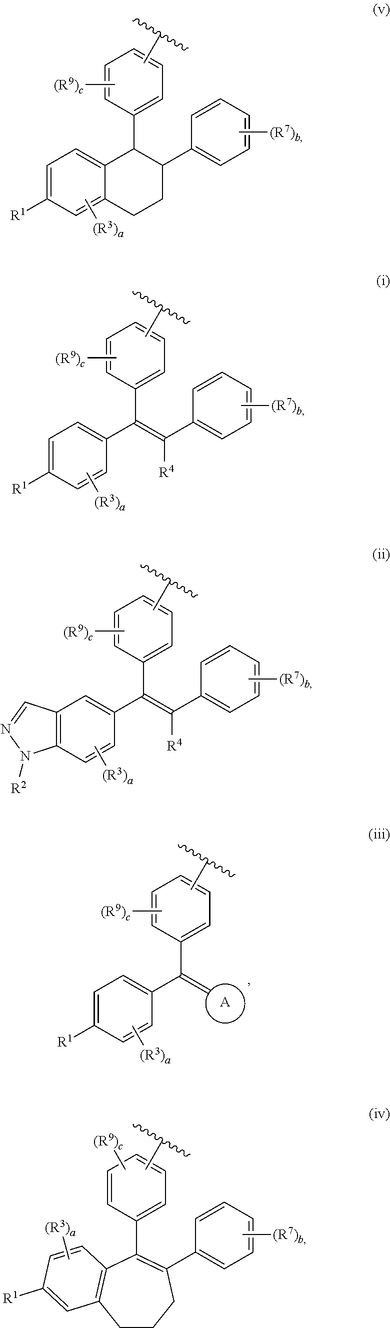 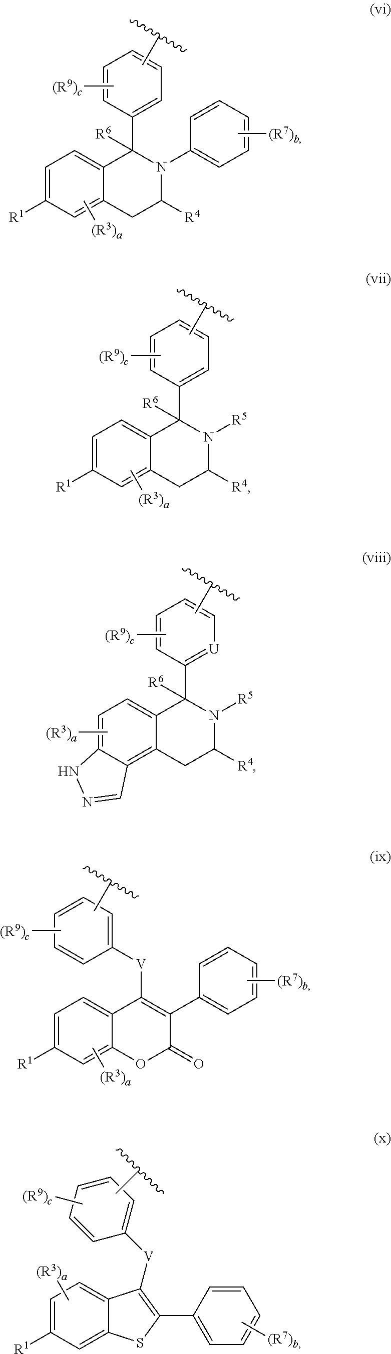 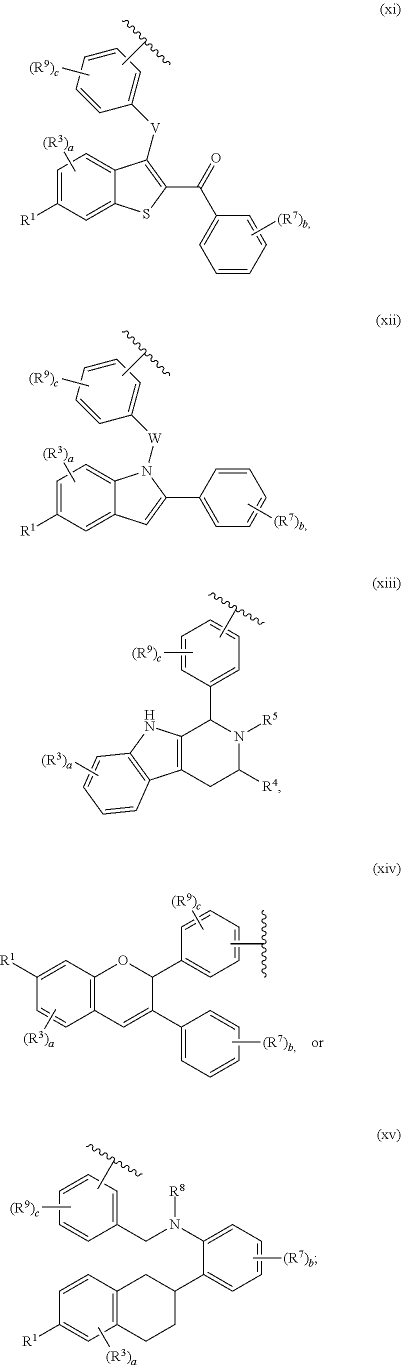
wherein:
ring A is C₃₋₁₀ cycloalkylene or heterocyclylene;
U is —CH═ or —N=;
each V is independently —O—, —S—, —N(R^{1a})—, —C(O)—, or C₁₋₆ alkylene;
W is —O—, —N(R^{1a})—, —C(O)—, or C₁₋₆ alkylene;
each R¹ is independently hydrogen, deuterium, halo, —C(O)OR^{1a}, —OR^{1a}, or —NR^{1b}R^{1c};
each R², R⁵, and R⁸ is independently (i) hydrogen; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c};
each R³, R⁷, and R⁹ is independently (i) hydrogen, deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each R⁴ and R⁶ is independently hydrogen, deuterium, C₁₋₆ alkyl, or C₁₋₆ heteroalkyl;
each a and c is independently an integer of 0, 1, 2, 3, or 4;
each b is independently an integer of 0, 1, 2, 3, 4, or 5; and
each R^{1a}, R^{1b}, R^{1c}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
wherein each alkyl, alkylene, heteroalkyl, alkenyl, alkynyl, cycloalkyl, cycloalkylene, aryl, aralkyl, heteroaryl, heterocyclyl, and heterocyclylene is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{b})OR^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OP(O)(OR^{f})OR^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

2. The compound of claim 1, having the structure of Formula (II): 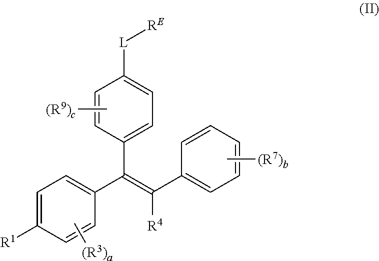
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

3. The compound of claim 1, having the structure of Formula (III): 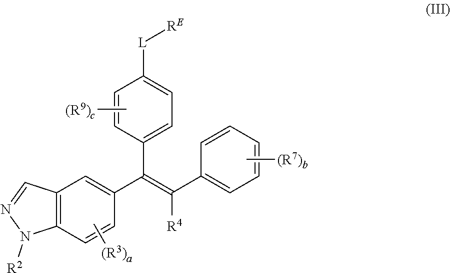
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

4. The compound of any one of claims 1 to 3, wherein R⁴ is C₁₋₆ alkyl or C₁₋₆ heteroalkyl, each optionally substituted with one or more substituents Q.

5. The compound of any one of claims 1 to 4, wherein R⁴ is ethyl or 2-chloroethyl.

6. The compound of any one of claims 1 and 3 to 5, wherein R² is hydrogen or C₁₋₆ alkyl, optionally substituted with one or more substituents Q.

7. The compound of any one of claims 1 and 3 to 6, wherein R² is hydrogen or methyl.

8. The compound of claim 1, having the structure of Formula (IV): 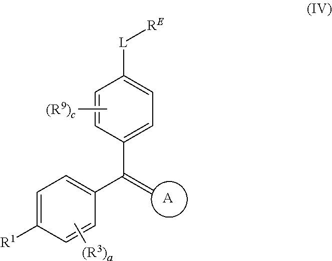
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

9. The compound of claim 1 or 8, wherein ring A is C₃₋₁₀ cycloalkylene, optionally substituted with one or more substituents Q.

10. The compound of claim 1, 8, or 9, wherein ring A is cyclohexan-1,1-diyl, bicyclo[3.3.1]nonan-9,9-diyl, or adamantan-2,2-diyl, each optionally substituted with one or more substituents Q.

11. The compound of claim 1, having the structure of Formula (V): 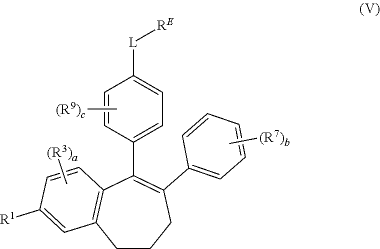
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

12. The compound of claim 1, having the structure of Formula (VI): 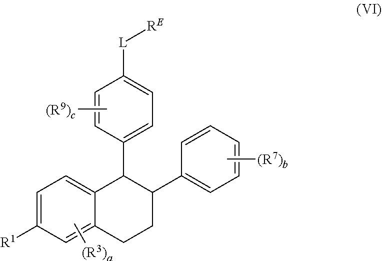
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

13. The compound of claim 1, having the structure of Formula (VII): 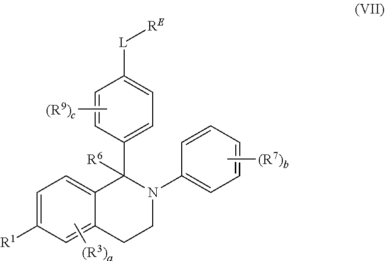
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

14. The compound of claim 1, having the structure of Formula (VIII): 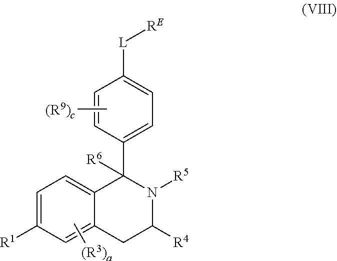
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

15. The compound of claim 1, having the structure of Formula (IX): 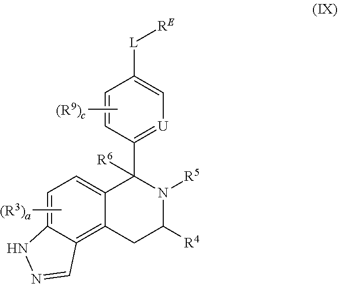
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

16. The compound of claim 1 or 15, wherein U is —CH═.

17. The compound of claim 1 or 15, wherein U is —N=.

18. The compound of any one of claims 1 and 14 to 17, wherein R⁴ is hydrogen, deuterium, or C₁₋₆ alkyl, optionally substituted with one or more substituents Q.

19. The compound of any one of claims 1 and 14 to 18, wherein R⁴ is hydrogen or methyl.

20. The compound of any one of claims 1 and 13 to 19, wherein R⁶ is hydrogen, deuterium, or C₁₋₆ alkyl, optionally substituted with one or more substituents Q.

21. The compound of any one of claims 1 and 13 to 20, wherein R⁶ is hydrogen or methyl.

22. The compound of claim 1, having the structure of Formula (X): 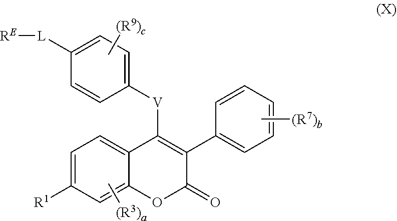
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

23. The compound of claim 1, having the structure of Formula (XI): 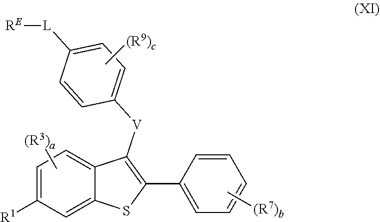
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

24. The compound of claim 1, having the structure of Formula (XII): 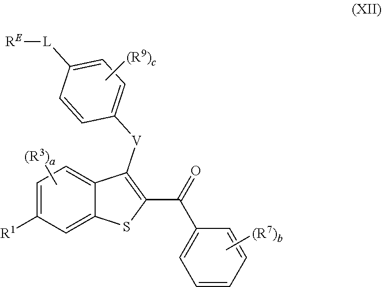
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

25. The compound of any one of claims 1 and 22 to 24, wherein V is —O—, —C(O)—, or C₁₋₆ alkylene, optionally substituted with one or more substituents Q.

26. The compound of any one of claims 1 and 22 to 25, wherein V is —O—.

27. The compound of any one of claims 1 and 22 to 25, wherein V is —C(O)—.

28. The compound of any one of claims 1 and 22 to 25, wherein V is methanediyl.

29. The compound of claim 1, having the structure of Formula (XIII): 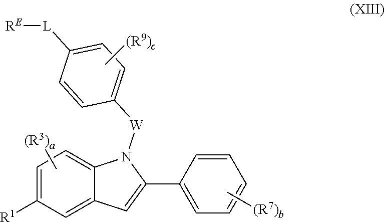
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

30. The compound of claim 1 or 29, wherein W is —C(O)— or C₁₋₆ alkylene, optionally substituted with one or more substituents Q.

31. The compound of claim 1, 29, or 30, wherein W is —C(O)— or methanediyl.

32. The compound of claim 1, having the structure of Formula (XIV): 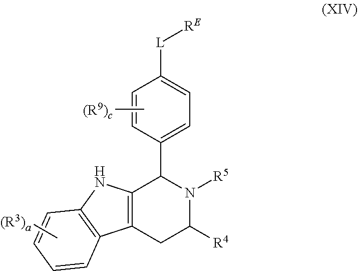
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

33. The compound of claim 1 or 32, wherein R⁴ is hydrogen, deuterium, or C₁₋₆ alkyl, optionally substituted with one or more substituents Q.

34. The compound of claim 1, 32, or 33, wherein R⁴ is hydrogen or methyl.

35. The compound of claim 1, having the structure of Formula (XV): 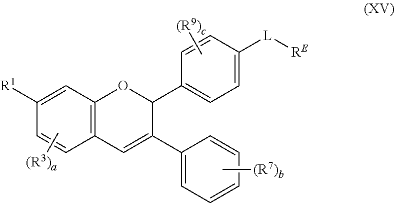
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

36. The compound of claim 1, having the structure of Formula (XVI): 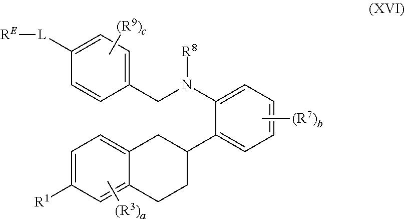
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

37. The compound of claim 1 or 36, wherein R⁸ is hydrogen or C₁₋₆ alkyl, optionally substituted with one or more substituents Q.

38. The compound of claim 1, 35, or 36, wherein R⁸ is hydrogen or ethyl.

39. The compound of any one of claims 1, 2, 4 to 14, 18 to 31, and 35 to 38, wherein R¹ is hydrogen, halo, —C(O)OR^{1a}, —OR^{1a}, or —NR^{1b}R^{1c}.

40. The compound of any one of claims 1, 2, 4 to 14, 18 to 31, and 35 to 39, wherein R¹ is hydrogen, fluoro, iodo, —CO₂H, —OH, or —NH₂.

41. The compound of any one of claims 1, 2, 4 to 14, 18 to 31, and 35 to 40, wherein R¹ is hydrogen.

42. The compound of any one of claims 1, 2, 4 to 14, 18 to 31, and 35 to 40, wherein R¹ is —CO₂H.

43. The compound of any one of claims 1, 2, 4 to 14, 18 to 31, and 35 to 40, wherein R¹ is —OH.

44. The compound of any one of claims 1 to 43, wherein a is an integer of 0, 1, or 2.

45. The compound of any one of claims 1 to 44, wherein each R³ is independently halo, C₁₋₆ alkyl, C₁₋₆ heteroalkyl, or —OR^{1a}, wherein the alkyl and heteroalkyl are optionally substituted with one or more substituents Q.

46. The compound of any one of claims 1 to 45, wherein each R³ is independently fluoro, chloro, hydroxyl, or methyl.

47. The compound of any one of claims 1, 14 to 21, 32 to 34, and 39 to 46, wherein R⁵ is C₁₋₆ alkyl or C₁₋₆ heteroalkyl, optionally substituted with one or more substituents Q.

48. The compound of any one of claims 1, 14 to 21, 32 to 34, and 39 to 46, wherein R⁵ is 2,2,2-trifluoroethyl, 2,2-difluoropropyl, 2,2-difluoro-3-hydroxylpropyl, isobutyl, 2-fluoroisobutyl, or benzyl.

49. The compound of any one of claims 1 to 7, 11 to 13, 18 to 31, and 35 to 46, wherein b is an integer of 0, 1, 2, or 3.

50. The compound of any one of claims 1 to 7, 11 to 13, 18 to 31, 35 to 46, and 49, wherein each R⁷ is independently halo, C₁₋₆ alkyl, C₁₋₆ heteroalkyl, or —OR^{1a}, wherein the alkyl and heteroalkyl are optionally substituted with one or more substituents Q.

51. The compound of any one of claims 1 to 7, 11 to 13, 18 to 31, 35 to 46, 49, and 50, wherein each R⁷ is independently fluoro, chloro, bromo, methyl, trifluoromethyl, ethyl, 2,2-filuoropropyl, isopropyl, isobutyl, t-butyl, cyclopropylmethyl, hydroxyl, methoxy, or trifluoromethoxy.

52. The compound of any one of claims 1 to 51, wherein c is an integer of 0, 1, or 2.

53. The compound of any one of claims 1 to 52, wherein each R⁹ is independently halo, C₁₋₆ alkyl, C₁₋₆ heteroalkyl, or —OR^{1a}, wherein the alkyl and heteroalkyl are optionally substituted with one or more substituents Q.

54. The compound of any one of claims 1 to 53, wherein each R⁹ is independently fluoro, methyl, or methoxy.

55. The compound of claim 1, wherein R^{T} is:

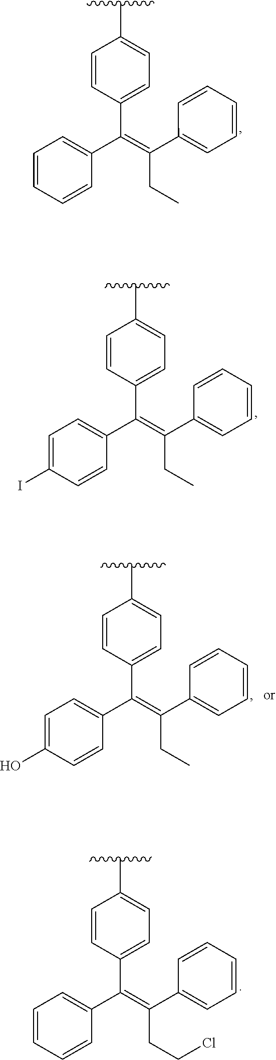 [SMILES: *c1ccc(/C(=C(/CC)c2ccccc2)c2ccccc2)cc1 ; *c1ccc(/C(=C(/CC)c2ccccc2)c2ccc(I)cc2)cc1 ; *c1ccc(/C(=C(/CC)c2ccccc2)c2ccc(O)cc2)cc1 ; *c1ccc(/C(=C(/CCCl)c2ccccc2)c2ccccc2)cc1]

56. The compound of claim 1, wherein R^{T} is:

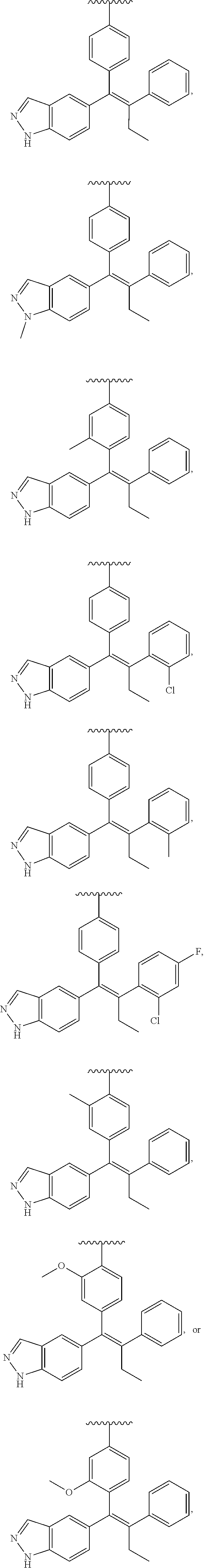

57. The compound of claim 1, wherein R^{T} is:

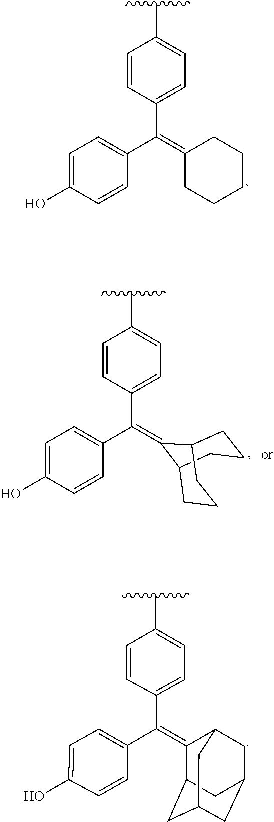 [SMILES: *c1ccc(C(=C2CCCCC2)c2ccc(O)cc2)cc1 ; *c1ccc(C(=C2C3CCCC2CCC3)c2ccc(O)cc2)cc1 ; *c1ccc(C(=C2C3CC4CC(C3)CC2C4)c2ccc(O)cc2)cc1]

58. The compound of claim 1, wherein R^{T} is:

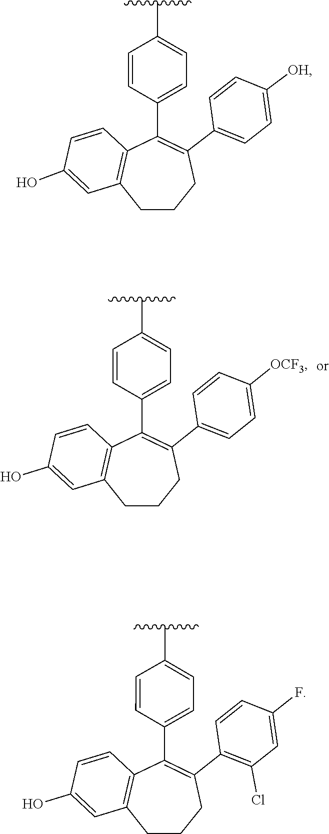 [SMILES: *c1ccc(C2=C(c3ccc(O)cc3)CCCc3cc(O)ccc32)cc1 ; *c1ccc(C2=C(c3ccc(OC(F)(F)F)cc3)CCCc3cc(O)ccc32)cc1 ; *c1ccc(C2=C(c3ccc(F)cc3Cl)CCCc3cc(O)ccc32)cc1]

59. The compound of claim 1, wherein R^{T} is:

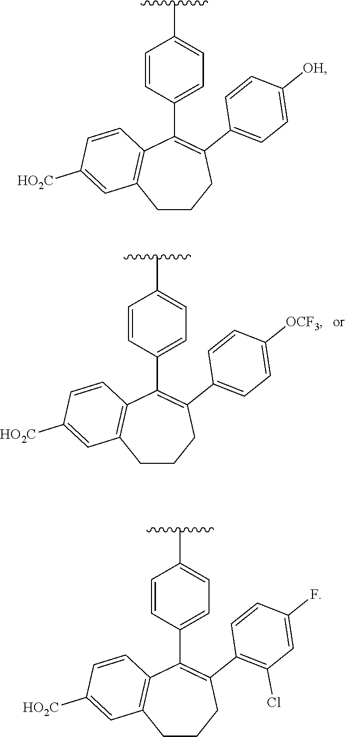 [SMILES: *c1ccc(C2=C(c3ccc(O)cc3)CCCc3cc(C(=O)O)ccc32)cc1 ; *c1ccc(C2=C(c3ccc(OC(F)(F)F)cc3)CCCc3cc(C(=O)O)ccc32)cc1 ; *c1ccc(C2=C(c3ccc(F)cc3Cl)CCCc3cc(C(=O)O)ccc32)cc1]

60. The compound of claim 1, wherein R^{T} is:

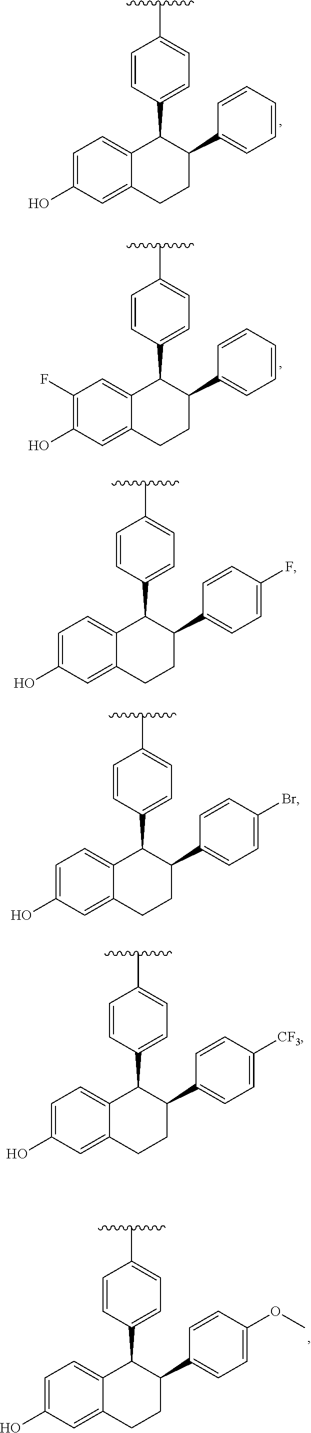

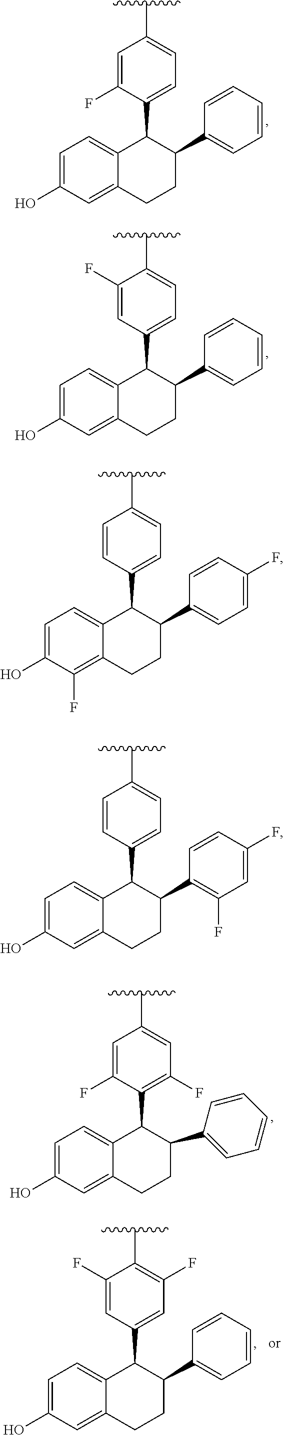

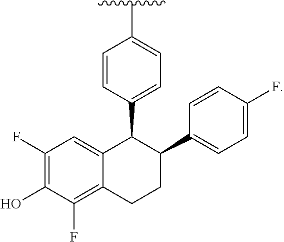 [SMILES: *c1ccc([C@@H]2c3cc(F)c(O)c(F)c3CC[C@@H]2c2ccc(F)cc2)cc1]

61. The compound of claim 1, wherein R^{T} is:

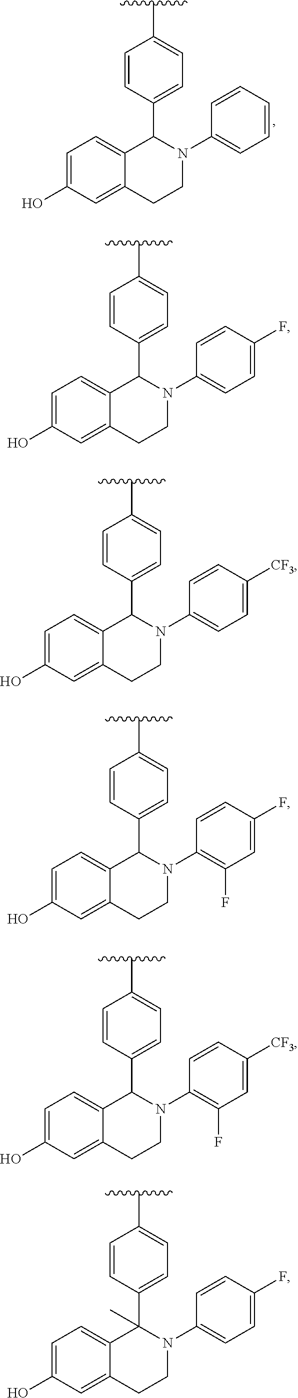

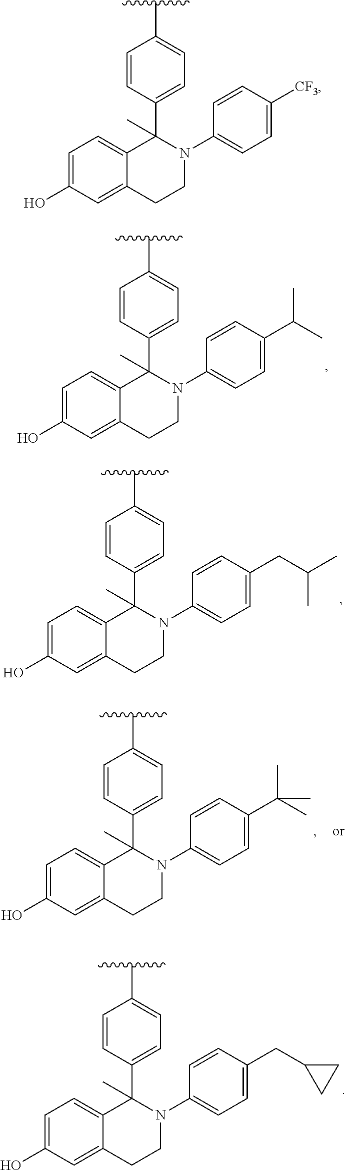

62. The compound of claim 1, wherein R^{T} is:

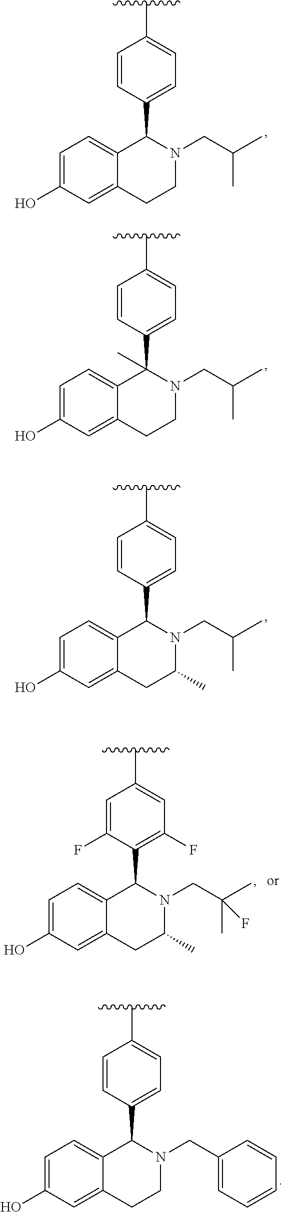

63. The compound of claim 1, wherein R^{T} is:

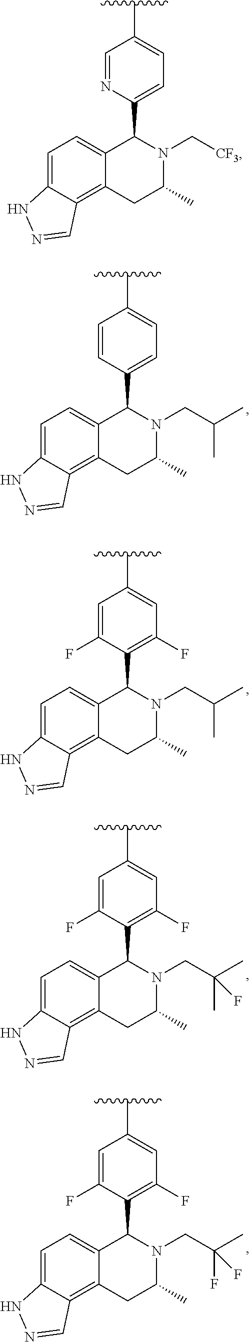 [SMILES: *c1ccc([C@@H]2c3ccc4[nH]ncc4c3C[C@@H](C)N2CC(F)(F)F)nc1 ; *c1ccc([C@@H]2c3ccc4[nH]ncc4c3C[C@@H](C)N2CC(C)C)cc1 ; *c1cc(F)c([C@@H]2c3ccc4[nH]ncc4c3C[C@@H](C)N2CC(C)C)c(F)c1 ; *c1cc(F)c([C@@H]2c3ccc4[nH]ncc4c3C[C@@H](C)N2CC(C)(C)F)c(F)c1 ; *c1cc(F)c([C@@H]2c3ccc4[nH]ncc4c3C[C@@H](C)N2CC(C)(F)F)c(F)c1]

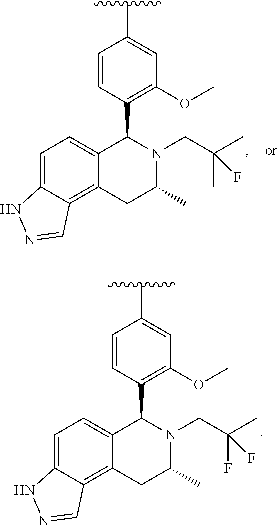 [SMILES: *c1ccc([C@@H]2c3ccc4[nH]ncc4c3C[C@@H](C)N2CC(C)(C)F)c(OC)c1 ; *c1ccc([C@@H]2c3ccc4[nH]ncc4c3C[C@@H](C)N2CC(C)(F)F)c(OC)c1]

64. The compound of claim 1, wherein R^{T} is:

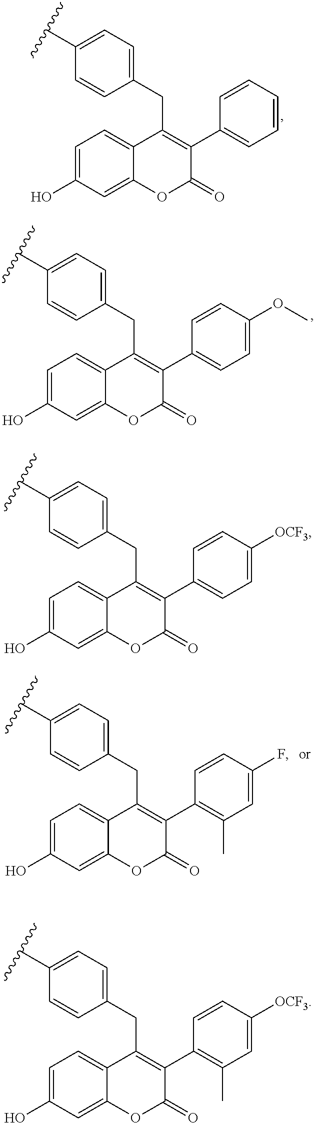 [SMILES: *c1ccc(Cc2c(-c3ccccc3)c(=O)oc3cc(O)ccc23)cc1 ; *c1ccc(Cc2c(-c3ccc(OC)cc3)c(=O)oc3cc(O)ccc23)cc1 ; *c1ccc(Cc2c(-c3ccc(OC(F)(F)F)cc3)c(=O)oc3cc(O)ccc23)cc1 ; *c1ccc(Cc2c(-c3ccc(F)cc3C)c(=O)oc3cc(O)ccc23)cc1 ; *c1ccc(Cc2c(-c3ccc(OC(F)(F)F)cc3C)c(=O)oc3cc(O)ccc23)cc1]

65. The compound of claim 1, wherein R^{T} is:

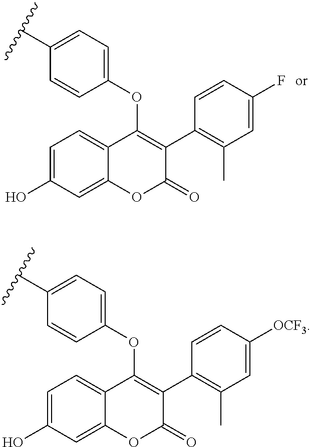 [SMILES: *c1ccc(Oc2c(-c3ccc(F)cc3C)c(=O)oc3cc(O)ccc23)cc1 ; *c1ccc(Oc2c(-c3ccc(OC(F)(F)F)cc3C)c(=O)oc3cc(O)ccc23)cc1]

66. The compound of claim 1, wherein R^{T} is:

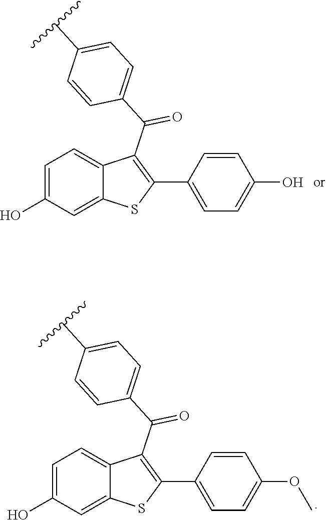 [SMILES: *c1ccc(C(=O)c2c(-c3ccc(O)cc3)sc3cc(O)ccc23)cc1 ; *c1ccc(C(=O)c2c(-c3ccc(OC)cc3)sc3cc(O)ccc23)cc1]

67. The compound of claim 1, wherein R^{T} is:

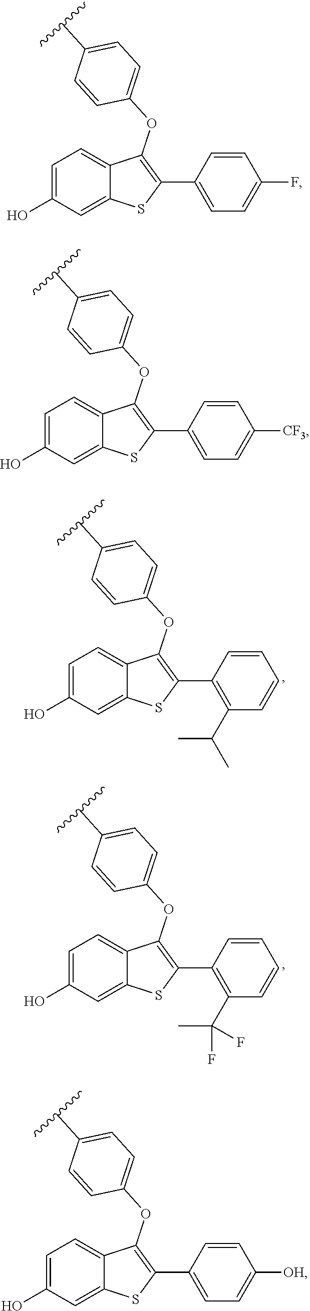 [SMILES: *c1ccc(Oc2c(-c3ccc(F)cc3)sc3cc(O)ccc23)cc1 ; *c1ccc(Oc2c(-c3ccc(C(F)(F)F)cc3)sc3cc(O)ccc23)cc1 ; *c1ccc(Oc2c(-c3ccccc3C(C)C)sc3cc(O)ccc23)cc1 ; *c1ccc(Oc2c(-c3ccccc3C(C)(F)F)sc3cc(O)ccc23)cc1 ; *c1ccc(Oc2c(-c3ccc(O)cc3)sc3cc(O)ccc23)cc1]

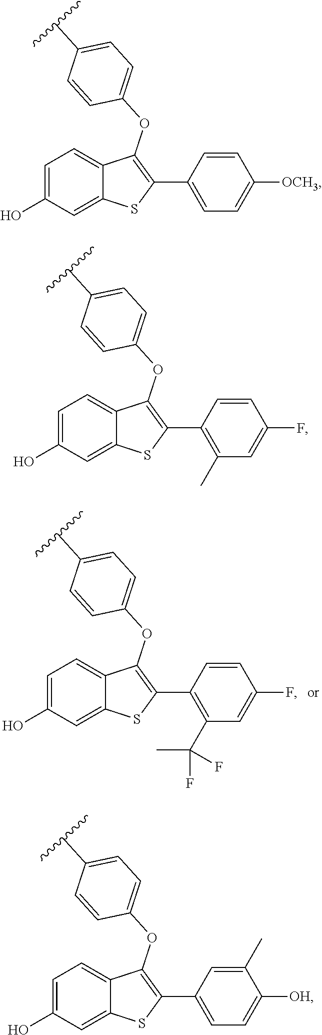

68. The compound of claim 1, wherein R^{T} is:

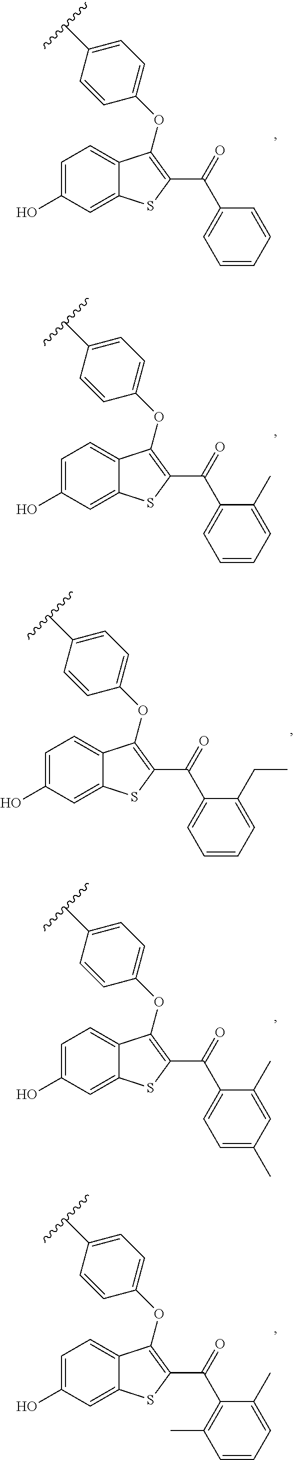 [SMILES: *c1ccc(Oc2c(C(=O)c3ccccc3)sc3cc(O)ccc23)cc1 ; *c1ccc(Oc2c(C(=O)c3ccccc3C)sc3cc(O)ccc23)cc1 ; *c1ccc(Oc2c(C(=O)c3ccccc3CC)sc3cc(O)ccc23)cc1 ; *c1ccc(Oc2c(C(=O)c3ccc(C)cc3C)sc3cc(O)ccc23)cc1 ; *c1ccc(Oc2c(C(=O)c3c(C)cccc3C)sc3cc(O)ccc23)cc1]

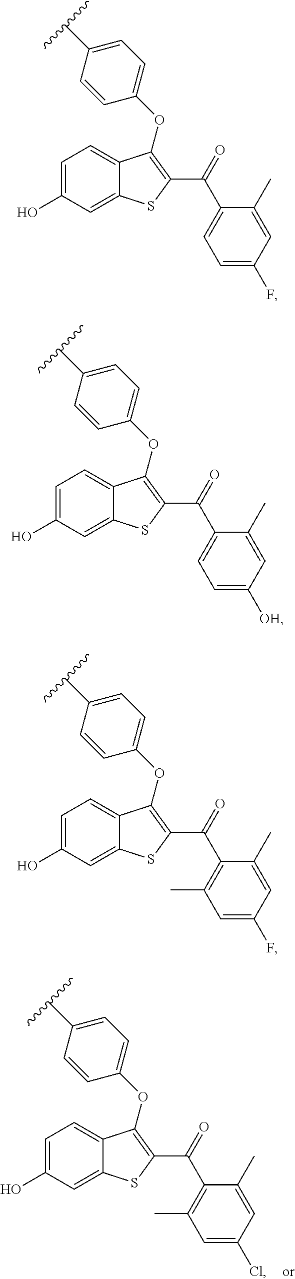 [SMILES: *c1ccc(Oc2c(C(=O)c3ccc(F)cc3C)sc3cc(O)ccc23)cc1 ; *c1ccc(Oc2c(C(=O)c3ccc(O)cc3C)sc3cc(O)ccc23)cc1 ; *c1ccc(Oc2c(C(=O)c3c(C)cc(F)cc3C)sc3cc(O)ccc23)cc1 ; *c1ccc(Oc2c(C(=O)c3c(C)cc(Cl)cc3C)sc3cc(O)ccc23)cc1]

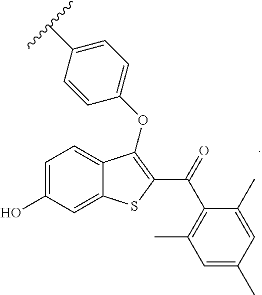 [SMILES: *c1ccc(Oc2c(C(=O)c3c(C)cc(C)cc3C)sc3cc(O)ccc23)cc1]

69. The compound of claim 1, wherein R^{T} is

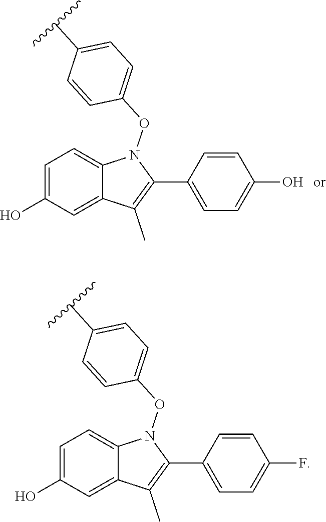 [SMILES: *c1ccc(On2c(-c3ccc(O)cc3)c(C)c3cc(O)ccc32)cc1 ; *c1ccc(On2c(-c3ccc(F)cc3)c(C)c3cc(O)ccc32)cc1]

70. The compound of claim 1, wherein R^{T} is:

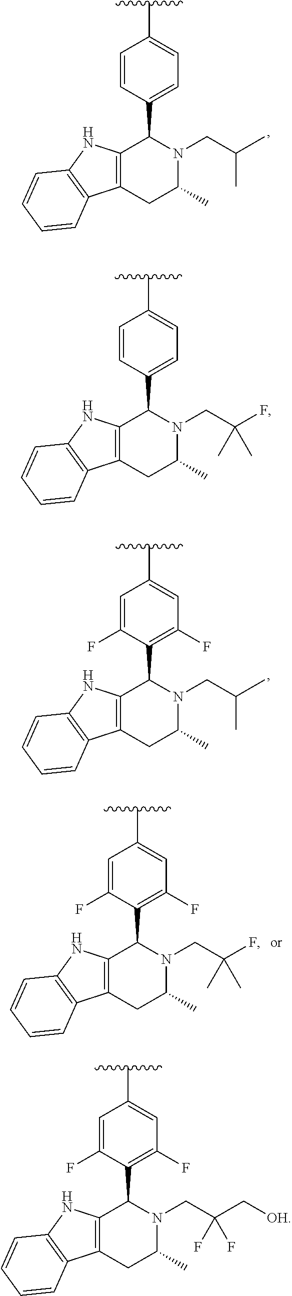 [SMILES: *c1ccc([C@@H]2c3[nH]c4ccccc4c3C[C@@H](C)N2CC(C)C)cc1 ; *c1ccc([C@@H]2c3[nH]c4ccccc4c3C[C@@H](C)N2CC(C)(C)F)cc1 ; *c1cc(F)c([C@@H]2c3[nH]c4ccccc4c3C[C@@H](C)N2CC(C)C)c(F)c1 ; *c1cc(F)c([C@@H]2c3[nH]c4ccccc4c3C[C@@H](C)N2CC(C)(C)F)c(F)c1 ; *c1cc(F)c([C@@H]2c3[nH]c4ccccc4c3C[C@@H](C)N2CC(F)(F)CO)c(F)c1]

71. The compound of claim 1, wherein R^{T} is:

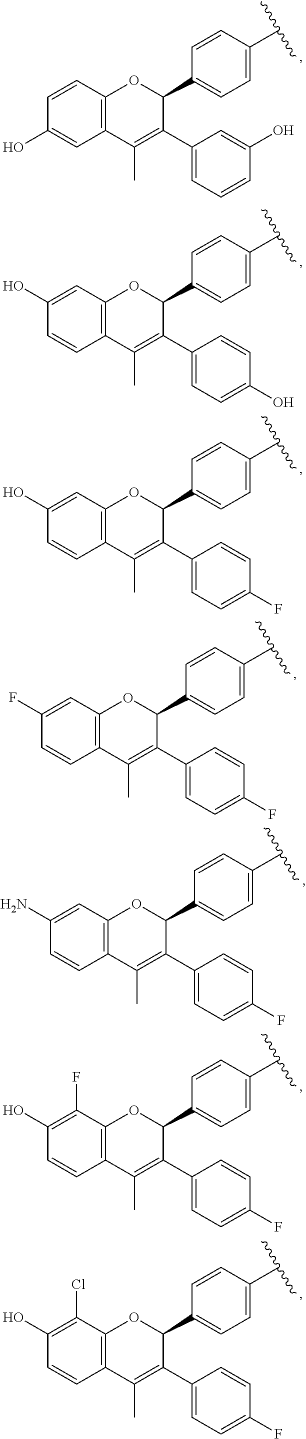

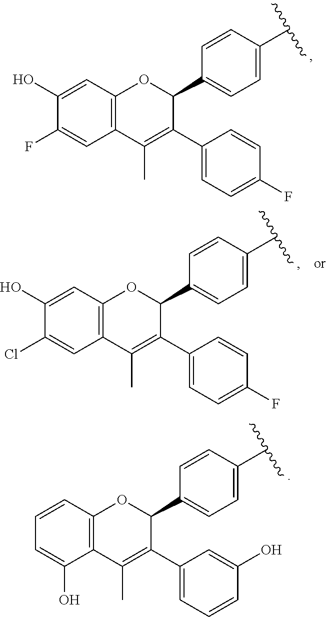

72. The compound of claim 1, wherein R^{T} is

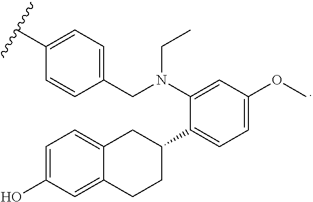 [SMILES: *c1ccc(CN(CC)c2cc(OC)ccc2[C@@H]2CCc3cc(O)ccc3C2)cc1]

73. The compound of any one of claims 1 to 72, wherein R^{E} is a moiety of a cereblon (CRBN) E3 ligand, an inhibitors-of-apoptosis protein (IAP) E3 ligand, a mouse double minute 2 homolog (MDM2) E3 ligand, or a von Hippel-Lindau (VHL) E3 ligand.

74. The compound of any one of claims 1 to 73, wherein R^{E} is a moiety of a cereblon (CRBN) E3 ligand.

75. The compound of any one of claims 1 to 74, wherein R^{E} is a moiety having the structure of Formula (EC-I): 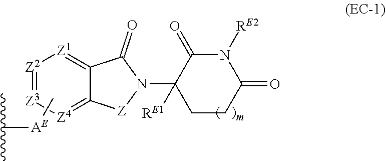
or an enantiomer, a mixture of enantiomers, a diastereomer, a mixture of two or more diastereomers, a tautomer, a mixture of two or more tautomers, or an isotopic variant thereof; wherein:
A^{E} is a bond, —O—, —N(R^{1b})—, —S—, C₁₋₆ alkylene, C₁₋₆ heteroalkylene, C₂₋₆ alkenylene, C₂₋₆ heteroalkenylene, C₂₋₆ alkynylene, C₂₋₆ heteroalkynylene, C₃₋₁₀ cycloalkylene, C₆₋₁₄ arylene, C₇₋₁₅ aralkylene, heteroarylene, heterocyclylene, C₁₋₆ heteroalkylene-C₆₋₁₄ arylene, C₁₋₆ heteroalkylene-heterocyclylene, or C₂₋₆ alkynylene-heterocyclylene;
Z is —CH₂— or —C(O)—;
one of Z¹, Z², Z³, and Z⁴ is —C═ and the remaining three of Z¹, Z², Z³, and Z⁴ are each independently —C(R^{E5})=; or Z¹ is a bond; one of Z², Z³, and Z⁴ is —C═, and the remaining two of Z², Z³, and Z⁴ are each independently —C(R^{E5})═ or —S—;
m is an integer of 0, 1, or 2;
R^{E1} is hydrogen, deuterium, halo, or C₁₋₆ alkyl;
R^{E2} is hydrogen or C₁₋₆ alkyl; and
each R^{E4} is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each R^{E5} is independently hydrogen or R^{E4};
wherein each alkyl, heteroalkyl, alkylene, heteroalkylene, alkenyl, alkenylene, heteroalkenylene, alkynyl, alkynylene, heteroalkynylene, cycloalkyl, cycloalkylene, aryl, arylene, aralkyl, aralkylene, heteroaryl, heteroarylene, heterocyclyl, and heterocyclylene is optionally substituted with one or more, in one embodiment, one, two, three, or four, substituents Q.

76. The compound of claim 75, wherein R^{E} is a moiety having the structure of Formula (EC-IV): 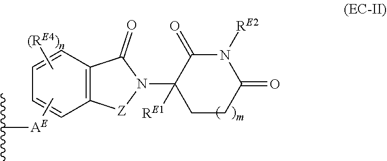
or an enantiomer, a mixture of enantiomers, a diastereomer, a mixture of two or more diastereomers, a tautomer, a mixture of two or more tautomers, or an isotopic variant thereof; wherein n is an integer of 0, 1, 2, or 3.

77. The compound of claim 75 or 76, wherein R^{E} is a moiety having the structure of Formula (EC-IV): 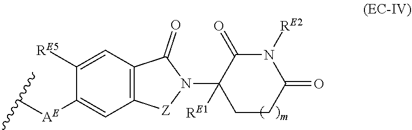
or an enantiomer, a mixture of enantiomers, a diastereomer, a mixture of two or more diastereomers, a tautomer, a mixture of two or more tautomers, or an isotopic variant thereof.

78. The compound of claim 75 or 76, wherein R^{E} is a moiety having the structure of Formula (EC-V): 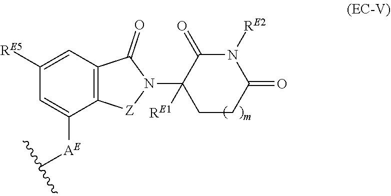
or an enantiomer, a mixture of enantiomers, a diastereomer, a mixture of two or more diastereomers, a tautomer, a mixture of two or more tautomers, or an isotopic variant thereof.

79. The compound of claim 75, wherein R^{E} is a moiety having the structure of Formula (EC-VI): 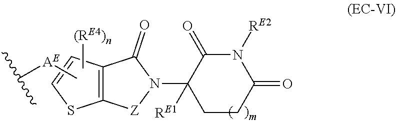
or an enantiomer, a mixture of enantiomers, a diastereomer, a mixture of two or more diastereomers, a tautomer, a mixture of two or more tautomers, or an isotopic variant thereof; wherein n is an integer of 0 or 1.

80. The compound of claim 75, wherein R^{E} is a moiety having the structure of Formula (EC-IX): 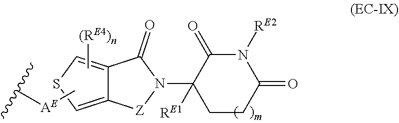
or an enantiomer, a mixture of enantiomers, a diastereomer, a mixture of two or more diastereomers, a tautomer, a mixture of two or more tautomers, or an isotopic variant thereof; wherein n is an integer of 0 or 1.

81. The compound of claim 75 or 80, wherein R^{E} is a moiety having the structure of Formula (EC-X): 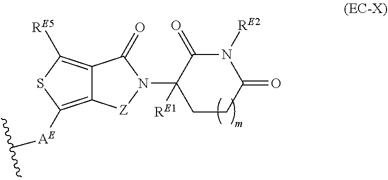
or an enantiomer, a mixture of enantiomers, a diastereomer, a mixture of two or more diastereomers, a tautomer, a mixture of two or more tautomers, or an isotopic variant thereof.

82. The compound of claim 75, wherein R^{E} is a moiety having the structure of Formula (EC-XII): 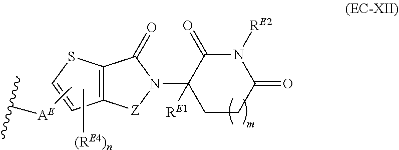
or an enantiomer, a mixture of enantiomers, a diastereomer, a mixture of two or more diastereomers, a tautomer, a mixture of two or more tautomers, or an isotopic variant thereof; wherein n is an integer of 0 or 1.

83. The compound of any one of claims 75 to 82, wherein Z is —CH₂—.

84. The compound of any one of claims 75 to 82, wherein Z is —C(O)—.

85. The compound of any one of claims 77, 78, 81, 83, and 84, wherein R^{E5} is hydrogen or fluoro.

86. The compound of any one of claims 1 to 74, wherein R^{E} is a moiety having the structure of Formula (EC-XV): 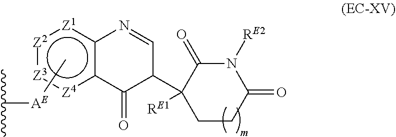
or an enantiomer, a mixture of enantiomers, a diastereomer, a mixture of two or more diastereomers, a tautomer, a mixture of two or more tautomers, or an isotopic variant thereof; wherein:
A^{E} is a bond, —O—, —N(R^{1b})—, —S—, C₁₋₆ alkylene, C₁₋₆ heteroalkylene, C₂₋₆ alkenylene, C₂₋₆ heteroalkenylene, C₂₋₆ alkynylene, C₂₋₆ heteroalkynylene, C₃₋₁₀ cycloalkylene, C₆₋₁₄ arylene, C₇₋₁₅ aralkylene, heteroarylene, heterocyclylene, C₁₋₆ heteroalkylene-C₆₋₁₄ arylene, C₁₋₆ heteroalkylene-heterocyclylene, or C₂₋₆ alkynylene-heterocyclylene;
one of Z¹, Z², Z³, and Z⁴ is —C═ and the remaining three of Z¹, Z², Z³, and Z⁴ are each independently —C(R^{E5})═; or Z¹ is a bond; one of Z², Z³, and Z⁴ is —C═, and the remaining two of Z², Z³, and Z⁴ are each independently —C(R^{E5})═ or —S—;
m is an integer of 0, 1, or 2;
R^{E1} is hydrogen, deuterium, halo, or C₁₋₆ alkyl;
R^{E2} is hydrogen or C₁₋₆ alkyl; and
R^{E3} is hydrogen, deuterium, halo, or C₁₋₆ alkyl;
each R^{E4} is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each R^{E5} is independently hydrogen or R^{E4};
wherein each alkyl, heteroalkyl, alkylene, heteroalkylene, alkenyl, alkenylene, heteroalkenylene, alkynyl, alkynylene, heteroalkynylene, cycloalkyl, cycloalkylene, aryl, arylene, aralkyl, aralkylene, heteroaryl, heteroarylene, heterocyclyl, and heterocyclylene is optionally substituted with one or more, in one embodiment, one, two, three, or four, substituents Q.

87. The compound of any one of claims 75 to 86, wherein R^{E2} is hydrogen.

88. The compound of any one of claims 75 to 87, wherein R^{E2} is hydrogen.

89. The compound of any one of claims 75 to 88, wherein m is an integer of 0.

90. The compound of any one of claims 75 to 88, wherein m is an integer of 1.

91. The compound of any one of claims 75 to 88, wherein m is an integer of 2.

92. The compound of any one of claims 1 to 75, wherein R^{E} is a moiety having the structure of: 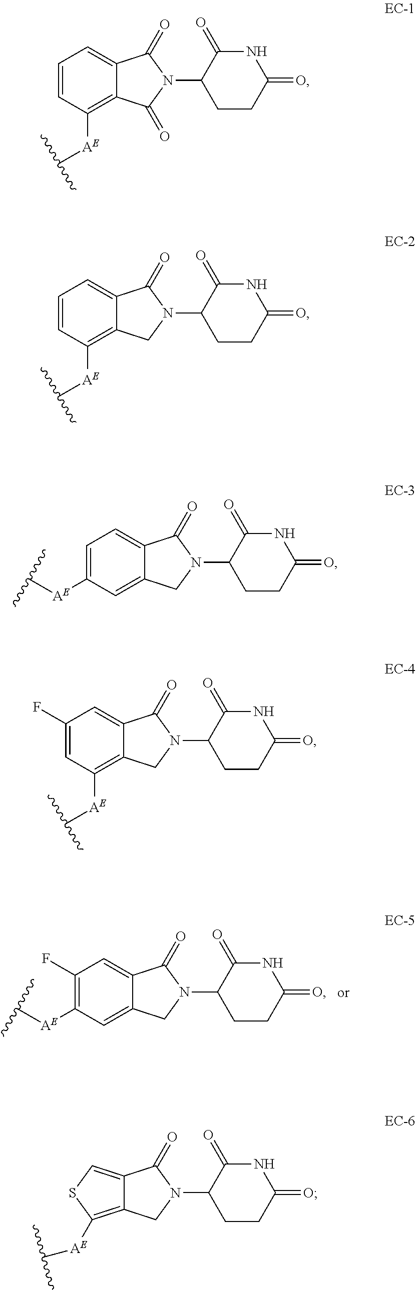
or an enantiomer, a mixture of enantiomers, a diastereomer, a mixture of two or more diastereomers, a tautomer, a mixture of two or more tautomers, or an isotopic variant thereof; wherein A^{E} is a bond, —O—, —N(R^{1b})—, —S—, C₁₋₆ alkylene, C₁₋₆ heteroalkylene, C₂₋₆ alkenylene, C₂₋₆ heteroalkenylene, C₂₋₆ alkynylene, C₂₋₆ heteroalkynylene, C₃₋₁₀ cycloalkylene, C₆₋₁₄ arylene, C₇₋₁₅ aralkylene, heteroarylene, heterocyclylene, C₁₋₆ heteroalkylene-C₆₋₁₄ arylene, or C₂₋₆ alkynylene-heterocyclylene; and wherein each alkylene, heteroalkylene, alkenylene, heteroalkenylene, alkynylene, heteroalkynylene, cycloalkylene, arylene, aralkylene, heteroarylene, and heterocyclylene is optionally substituted with one or more substituents Q.

93. The compound of any one of claims 75 to 92, wherein A^{E} is a bond, —O—, —N(R^{1b})—, C₂₋₆ alkynylene, heterocyclylene, C₁₋₆ heteroalkylene-C₆₋₁₄ arylene, or C₂₋₆ alkynylene-heterocyclylene, where each heteroalkylene, alkynylene, arylene, and heterocyclylene is optionally substituted with one or more substituents Q;

94. The compound of any one of claims 75 to 93, wherein A^{E} is a bond, —O—, —NH—, ethynediyl, piperidindiyl, piperazindiyl, (phendiyl)oxymethanediyl, or (piperidindiyl)ethynediyl.

95. The compound of any one of claims 75 to 94, wherein A^{E} is a bond, —NH—, piperidin-1,4-diyl, piperaz-1,4-diyl, (phen-1,4-diyl)oxymethanediyl, or (piperidin-1,4-diyl)ethynediyl.

96. The compound of any one of claims 1 to 75, wherein R^{E} is a moiety having the structure of: 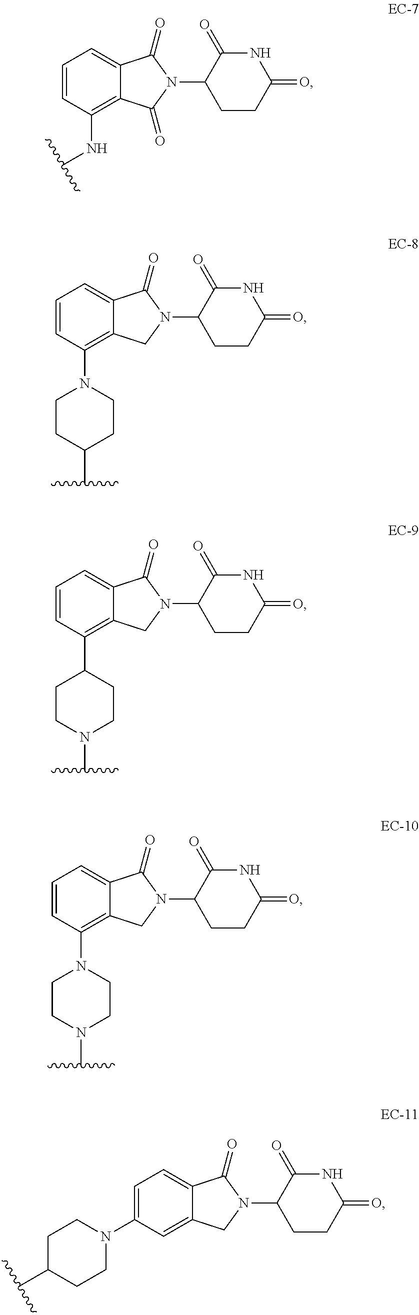 [SMILES: *Nc1cccc2c1C(=O)N(C1CCC(=O)NC1=O)C2=O ; *C1CCN(c2cccc3c2CN(C2CCC(=O)NC2=O)C3=O)CC1 ; *N1CCC(c2cccc3c2CN(C2CCC(=O)NC2=O)C3=O)CC1 ; *N1CCN(c2cccc3c2CN(C2CCC(=O)NC2=O)C3=O)CC1 ; *C1CCN(c2ccc3c(c2)CN(C2CCC(=O)NC2=O)C3=O)CC1] 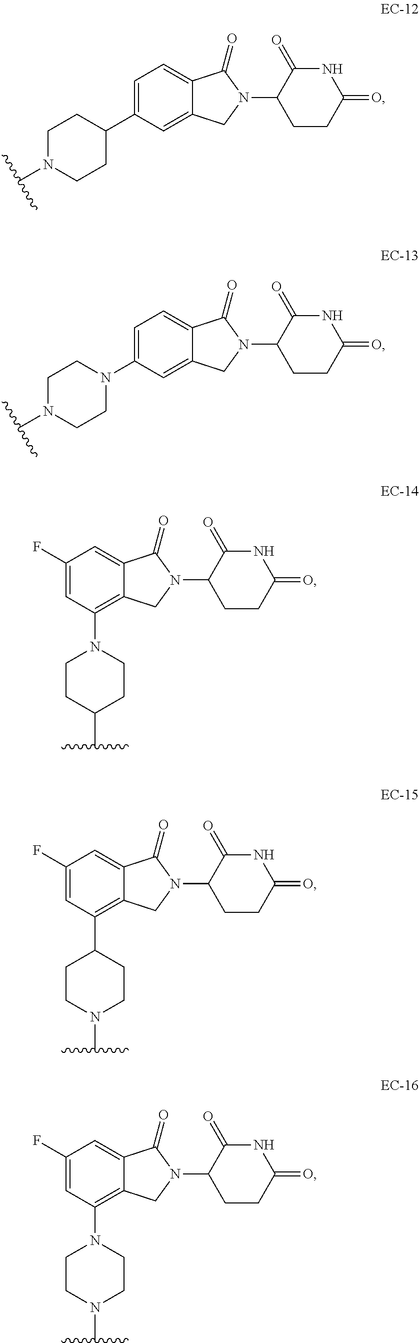 [SMILES: *N1CCC(c2ccc3c(c2)CN(C2CCC(=O)NC2=O)C3=O)CC1 ; *N1CCN(c2ccc3c(c2)CN(C2CCC(=O)NC2=O)C3=O)CC1 ; *C1CCN(c2cc(F)cc3c2CN(C2CCC(=O)NC2=O)C3=O)CC1 ; *N1CCC(c2cc(F)cc3c2CN(C2CCC(=O)NC2=O)C3=O)CC1 ; *N1CCN(c2cc(F)cc3c2CN(C2CCC(=O)NC2=O)C3=O)CC1] 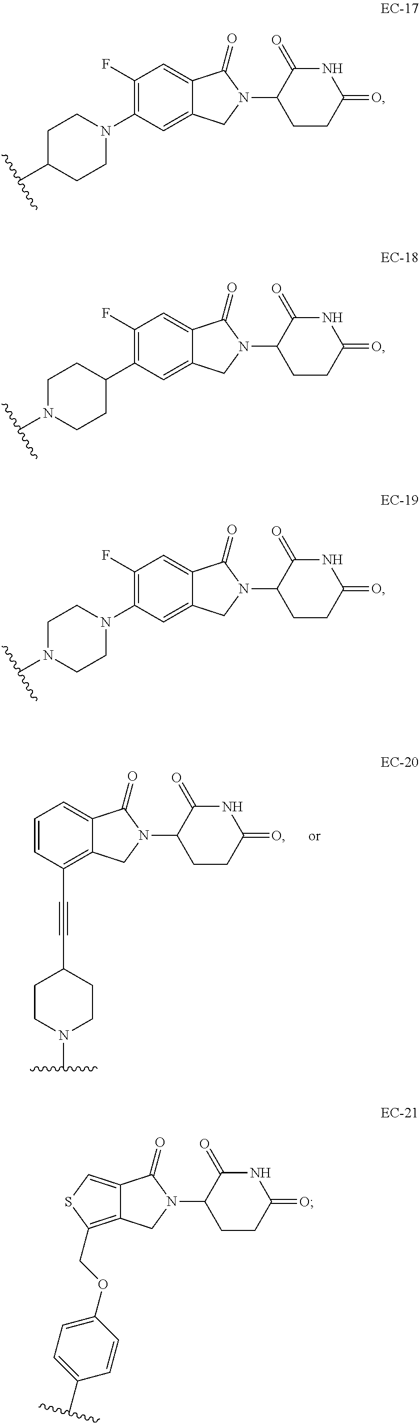 [SMILES: *C1CCN(c2cc3c(cc2F)C(=O)N(C2CCC(=O)NC2=O)C3)CC1 ; *N1CCC(c2cc3c(cc2F)C(=O)N(C2CCC(=O)NC2=O)C3)CC1 ; *N1CCN(c2cc3c(cc2F)C(=O)N(C2CCC(=O)NC2=O)C3)CC1 ; *N1CCC(C#Cc2cccc3c2CN(C2CCC(=O)NC2=O)C3=O)CC1 ; *c1ccc(OCc2scc3c2CN(C2CCC(=O)NC2=O)C3=O)cc1]
or an enantiomer, a mixture of enantiomers, a tautomer, a mixture of two or more tautomers, or an isotopic variant thereof.

97. The compound of any one of claims 1 to 75 and 96, wherein R^{E} is a moiety having the structure of: 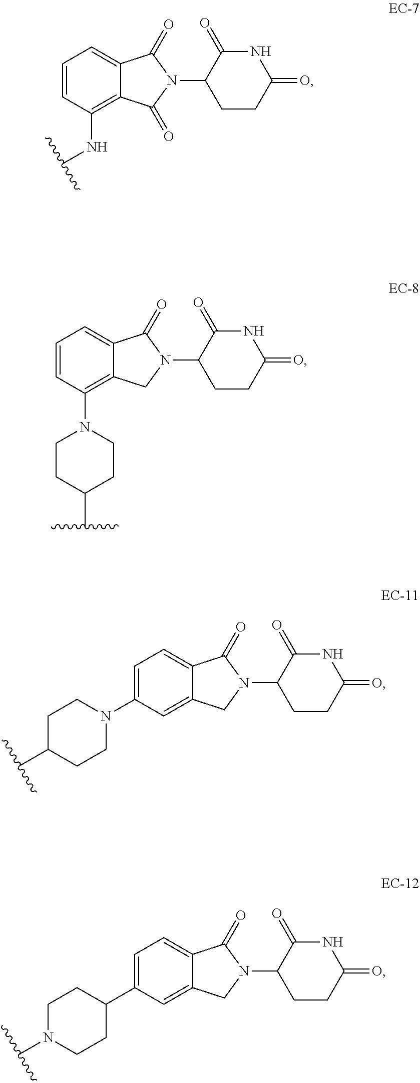 [SMILES: *Nc1cccc2c1C(=O)N(C1CCC(=O)NC1=O)C2=O ; *C1CCN(c2cccc3c2CN(C2CCC(=O)NC2=O)C3=O)CC1 ; *C1CCN(c2ccc3c(c2)CN(C2CCC(=O)NC2=O)C3=O)CC1 ; *N1CCC(c2ccc3c(c2)CN(C2CCC(=O)NC2=O)C3=O)CC1] 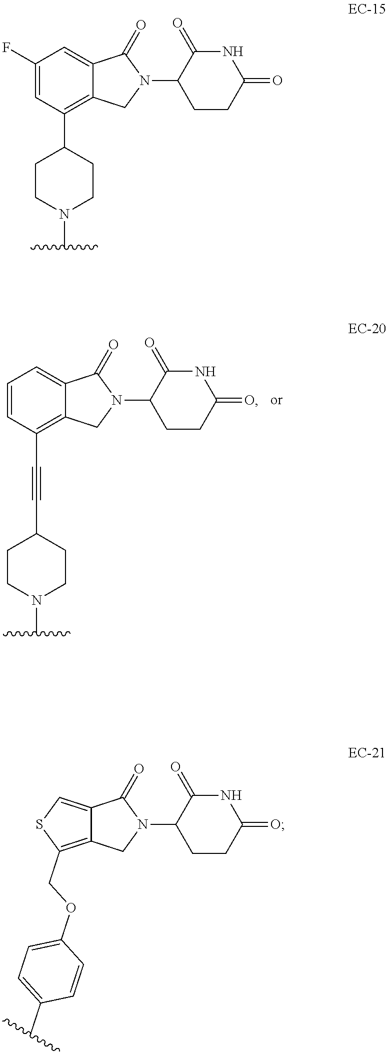 [SMILES: *N1CCC(c2cc(F)cc3c2CN(C2CCC(=O)NC2=O)C3=O)CC1 ; *N1CCC(C#Cc2cccc3c2CN(C2CCC(=O)NC2=O)C3=O)CC1 ; *c1ccc(OCc2scc3c2CN(C2CCC(=O)NC2=O)C3=O)cc1]
or an enantiomer, a mixture of enantiomers, a tautomer, a mixture of two or more tautomers, or an isotopic variant thereof.

98. The compound of any one of claims 1 to 75 and 96, wherein R^{E} is a moiety having the structure of: 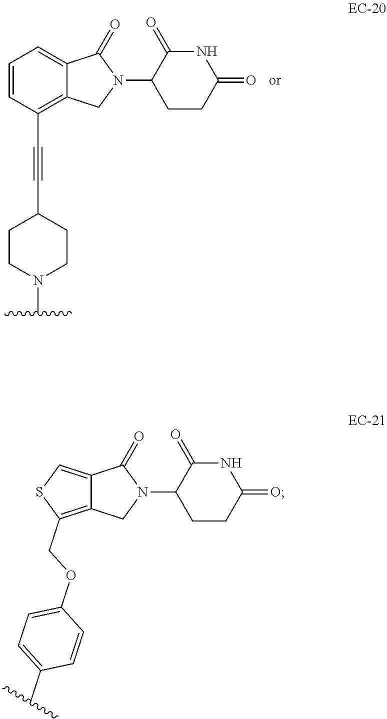 [SMILES: *N1CCC(C#Cc2cccc3c2CN(C2CCC(=O)NC2=O)C3=O)CC1 ; *c1ccc(OCc2scc3c2CN(C2CCC(=O)NC2=O)C3=O)cc1]
or an enantiomer, a mixture of enantiomers, a tautomer, a mixture of two or more tautomers, or an isotopic variant thereof.

99. The compound of any one of claims 1 to 74, wherein R^{E} is a moiety having the structure of: 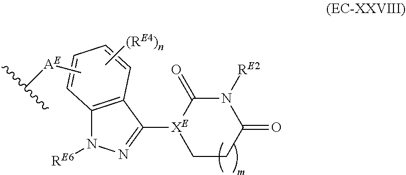
or an enantiomer, a mixture of enantiomers, a diastereomer, a mixture of two or more diastereomers, a tautomer, a mixture of two or more tautomers, or an isotopic variant thereof; wherein:
A^{E} is a bond, —O—, —N(R^{1b})—, —S—, C₁₋₆ alkylene, C₁₋₆ heteroalkylene, C₂₋₆ alkenylene, C₂₋₆ heteroalkenylene, C₂₋₆ alkynylene, C₂₋₆ heteroalkynylene, C₃₋₁₀ cycloalkylene, C₆₋₁₄ arylene, C₇₋₁₅ aralkylene, heteroarylene, heterocyclylene, C₁₋₆ heteroalkylene-C₆₋₁₄ arylene, C₁₋₆ heteroalkylene-heterocyclylene, or C₂₋₆ alkynylene-heterocyclylene;
X^{E} is C(R^{E}1) or N;
R^{E1} is hydrogen, deuterium, halo, or C₁₋₆ alkyl;
R^{E2} is hydrogen or C₁₋₆ alkyl;
each R^{E4} is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{E6} is (i) hydrogen; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, or three, substituents Q; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c};
m is an integer of 0, 1, or 2; and
n is an integer of 0, 1, 2, or 3;
wherein each alkyl, heteroalkyl, alkylene, heteroalkylene, alkenyl, alkenylene, heteroalkenylene, alkynyl, alkynylene, heteroalkynylene, cycloalkyl, cycloalkylene, aryl, arylene, aralkyl, aralkylene, heteroaryl, heteroarylene, heterocyclyl, and heterocyclylene is optionally substituted with one or more, in one embodiment, one, two, three, or four, substituents Q.

100. The compound of claim 99, wherein R^{E} is a moiety having the structure of Formula (EC-XXIX): 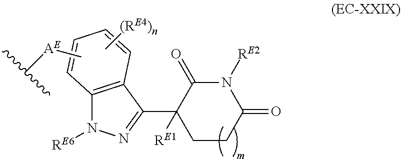
or an enantiomer, a mixture of enantiomers, a diastereomer, a mixture of two or more diastereomers, a tautomer, a mixture of two or more tautomers, or an isotopic variant thereof.

101. The compound of claim 99 or 100, wherein R^{E} is a moiety having the structure of Formula (EC-XXX): 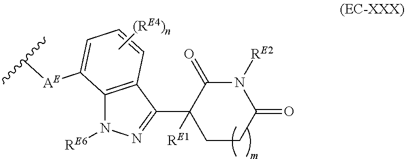
or an enantiomer, a mixture of enantiomers, a diastereomer, a mixture of two or more diastereomers, a tautomer, a mixture of two or more tautomers, or an isotopic variant thereof.

102. The compound of claim 99 or 100, wherein R^{E} is a moiety having the structure of Formula (EC-XXXI): 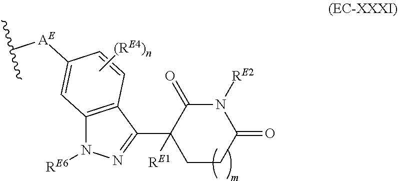
or an enantiomer, a mixture of enantiomers, a diastereomer, a mixture of two or more diastereomers, a tautomer, a mixture of two or more tautomers, or an isotopic variant thereof.

103. The compound of any one of claims 99 to 102, wherein R^{E1} is hydrogen.

104. The compound of claim 99, wherein R^{E} is a moiety having the structure of Formula (EC-XXXII): 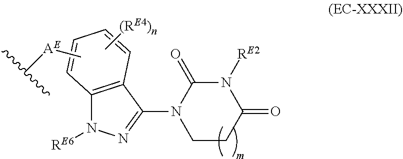
or an enantiomer, a mixture of enantiomers, a diastereomer, a mixture of two or more diastereomers, a tautomer, a mixture of two or more tautomers, or an isotopic variant thereof.

105. The compound of claim 99 or 104, wherein R^{E} is a moiety having the structure of Formula (EC-XXXIII): 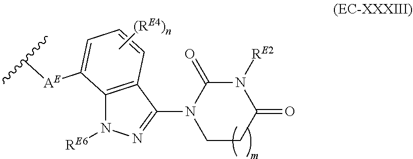
or an enantiomer, a mixture of enantiomers, a diastereomer, a mixture of two or more diastereomers, a tautomer, a mixture of two or more tautomers, or an isotopic variant thereof.

106. The compound of claim 99 or 104, wherein R^{E} is a moiety having the structure of Formula (EC-XXXIV): 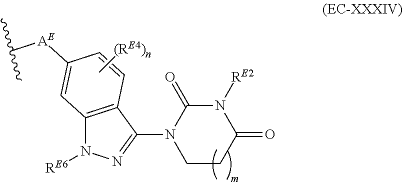
or an enantiomer, a mixture of enantiomers, a diastereomer, a mixture of two or more diastereomers, a tautomer, a mixture of two or more tautomers, or an isotopic variant thereof.

107. The compound of any one of claims 99 to 106, wherein R^{E2} is hydrogen.

108. The compound of any one of claims 99 to 107, wherein R^{E6} is C₁₋₆ alkyl, C₁₋₆ heteroalkyl.

109. The compound of any one of claims 99 to 108, wherein R^{E6} is methyl.

110. The compound of any one of claims 1 to 74 and 99, wherein R^{E} is a moiety having the structure of: 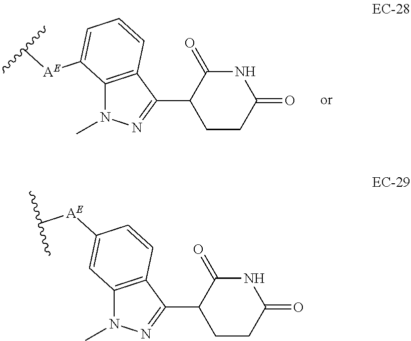
or an enantiomer, a mixture of enantiomers, a diastereomer, a mixture of two or more diastereomers, a tautomer, a mixture of two or more tautomers, or an isotopic variant thereof; wherein A^{E} is a bond, —O—, —N(R^{1b})—, —S—, C₁₋₆ alkylene, C₁₋₆ heteroalkylene, C₂₋₆ alkenylene, C₂₋₆ heteroalkenylene, C₂₋₆ alkynylene, C₂₋₆ heteroalkynylene, C₃₋₁₀ cycloalkylene, C₆₋₁₄ arylene, C₇₋₁₅ aralkylene, heteroarylene, heterocyclylene, C₁₋₆ heteroalkylene-C₆₋₁₄ arylene, C₁₋₆ heteroalkylene-heterocyclylene, or C₂₋₆ alkynylene-heterocyclylene; and wherein each alkylene, heteroalkylene, alkenylene, heteroalkenylene, alkynylene, heteroalkynylene, cycloalkylene, arylene, aralkylene, heteroarylene, and heterocyclylene is optionally substituted with one or more substituents Q.

111. The compound of any one of claims 1 to 74 and 110, wherein R^{E} is a moiety having the structure of: 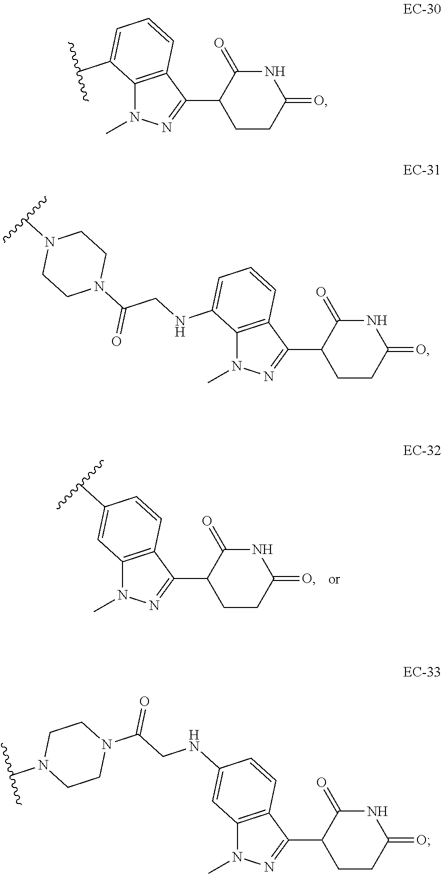 [SMILES: *c1cccc2c(C3CCC(=O)NC3=O)nn(C)c12 ; *N1CCN(C(=O)CNc2cccc3c(C4CCC(=O)NC4=O)nn(C)c23)CC1 ; *c1ccc2c(C3CCC(=O)NC3=O)nn(C)c2c1 ; *N1CCN(C(=O)CNc2ccc3c(C4CCC(=O)NC4=O)nn(C)c3c2)CC1]
or an enantiomer, a mixture of enantiomers, a tautomer, a mixture of two or more tautomers, or an isotopic variant thereof.

112. The compound of any one of claims 1 to 74, wherein R^{E} is a moiety having the structure of: 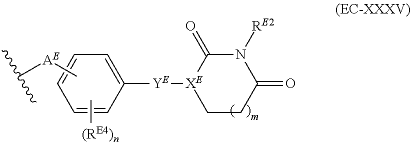
or an enantiomer, a mixture of enantiomers, a diastereomer, a mixture of two or more diastereomers, a tautomer, a mixture of two or more tautomers, or an isotopic variant thereof; wherein:
A^{E} is a bond, —O—, —N(R^{1b})—, —S—, C₁₋₆ alkylene, C₁₋₆ heteroalkylene, C₂₋₆ alkenylene, C₂₋₆ heteroalkenylene, C₂₋₆ alkynylene, C₂₋₆ heteroalkynylene, C₃₋₁₀ cycloalkylene, C₆₋₁₄ arylene, C₇₋₁₅ aralkylene, heteroarylene, heterocyclylene, C₁₋₆ heteroalkylene-C₆₋₁₄ arylene, C₁₋₆ heteroalkylene-heterocyclylene, or C₂₋₆ alkynylene-heterocyclylene;
X^{E} is C(R^{E1}) or N;
Y^{E} is a bond, C₁₋₆ alkylene, —O—, —S—, —S(O)—, —S(O₂)—, or —N(R^{E7})—;
R^{E1} is hydrogen, deuterium, halo, or C₁₋₆ alkyl;
R^{E2} is hydrogen or C₁₋₆ alkyl;
each R^{E4} is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{E7} is hydrogen or C₁₋₆ alkyl;
m is an integer of 0, 1, or 2; and
n is an integer of 0, 1, 2, 3, or 4;
wherein each alkyl, heteroalkyl, alkylene, heteroalkylene, alkenyl, alkenylene, heteroalkenylene, alkynyl, alkynylene, heteroalkynylene, cycloalkyl, cycloalkylene, aryl, arylene, aralkyl, aralkylene, heteroaryl, heteroarylene, heterocyclyl, and heterocyclylene is optionally substituted with one or more, in one embodiment, one, two, three, or four, substituents Q.

113. The compound of claim 112, wherein R^{E} is a moiety having the structure of Formula (EC-XXXVI): 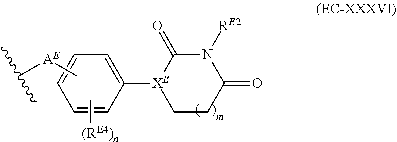
or an enantiomer, a mixture of enantiomers, a diastereomer, a mixture of two or more diastereomers, a tautomer, a mixture of two or more tautomers, or an isotopic variant thereof.

114. The compound of claim 112 or 113, wherein R^{E} is a moiety having the structure of Formula (EC-XXXVII): 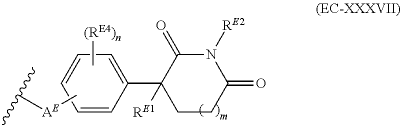
or an enantiomer, a mixture of enantiomers, a diastereomer, a mixture of two or more diastereomers, a tautomer, a mixture of two or more tautomers, or an isotopic variant thereof.

115. The compound of any one of claims 112 to 114, wherein R^{E} is a moiety having the structure of Formula (EC-XXXVIII): 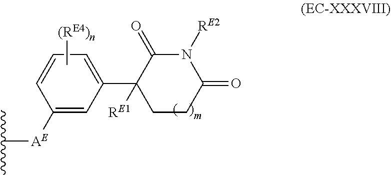
or an enantiomer, a mixture of enantiomers, a diastereomer, a mixture of two or more diastereomers, a tautomer, a mixture of two or more tautomers, or an isotopic variant thereof.

116. The compound of any one of claims 112 to 114, wherein R^{E} is a moiety having the structure of Formula (EC-XXXI): 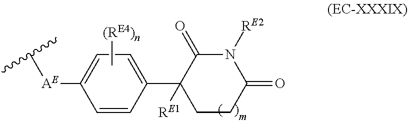
or an enantiomer, a mixture of enantiomers, a diastereomer, a mixture of two or more diastereomers, a tautomer, a mixture of two or more tautomers, or an isotopic variant thereof.

117. The compound of any one of claims 112 to 116, wherein R^{E1} is hydrogen.

118. The compound of claim 112 or 113, wherein R^{E} is a moiety having the structure of Formula (EC-XLI): 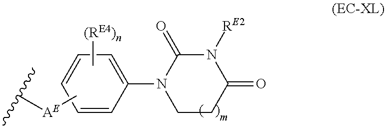
or an enantiomer, a mixture of enantiomers, a diastereomer, a mixture of two or more diastereomers, a tautomer, a mixture of two or more tautomers, or an isotopic variant thereof.

119. The compound of claim 112, 113, or 118, wherein R^{E} is a moiety having the structure of Formula (EC-XLI): 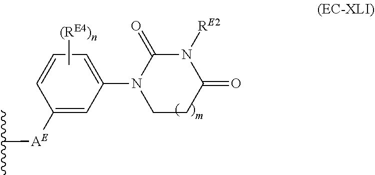
or an enantiomer, a mixture of enantiomers, a diastereomer, a mixture of two or more diastereomers, a tautomer, a mixture of two or more tautomers, or an isotopic variant thereof.

120. The compound of claim 112, 113, or 118, wherein R^{E} is a moiety having the structure of Formula (EC-XLII): 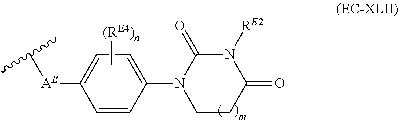
or an enantiomer, a mixture of enantiomers, a diastereomer, a mixture of two or more diastereomers, a tautomer, a mixture of two or more tautomers, or an isotopic variant thereof.

121. The compound of claim 112, wherein R^{E} is a moiety having the structure of Formula (EC-XLIII): 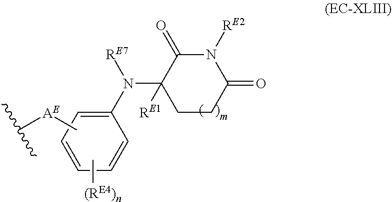
or an enantiomer, a mixture of enantiomers, a diastereomer, a mixture of two or more diastereomers, a tautomer, a mixture of two or more tautomers, or an isotopic variant thereof.

122. The compound of claim 112 or 121, wherein R^{E} is a moiety having the structure of Formula (EC-XLIV): 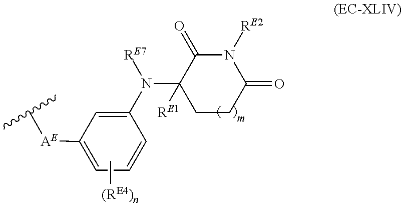
or an enantiomer, a mixture of enantiomers, a diastereomer, a mixture of two or more diastereomers, a tautomer, a mixture of two or more tautomers, or an isotopic variant thereof.

123. The compound of claim 112 or 121, wherein R^{E} is a moiety having the structure of Formula (EC-XLV): 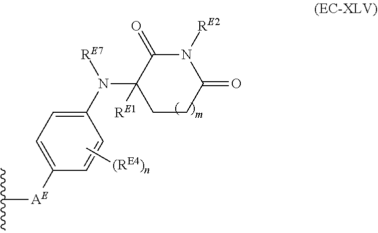
or an enantiomer, a mixture of enantiomers, a diastereomer, a mixture of two or more diastereomers, a tautomer, a mixture of two or more tautomers, or an isotopic variant thereof.

124. The compound of any one of claims 121 to 123, wherein R^{E1} is hydrogen.

125. The compound of any one of claims 121 to 124, wherein R^{E7} is hydrogen.

126. The compound of any one of claims 112 to 125, wherein R^{E2} is hydrogen.

127. The compound of any one of claims 1 to 74 and 112, wherein R^{E} is a moiety having the structure of: 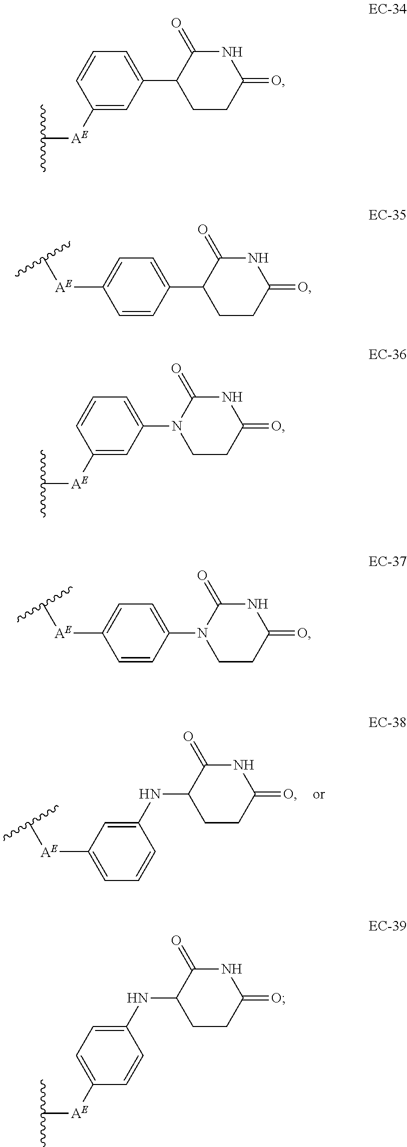
or an enantiomer, a mixture of enantiomers, a diastereomer, a mixture of two or more diastereomers, a tautomer, a mixture of two or more tautomers, or an isotopic variant thereof; wherein A^{E} is a bond, —O—, —N(R^{1b})—, —S—, C₁₋₆ alkylene, C₁₋₆ heteroalkylene, C₂₋₆ alkenylene, C₂₋₆ heteroalkenylene, C₂₋₆ alkynylene, C₂₋₆ heteroalkynylene, C₃₋₁₀ cycloalkylene, C₆₋₁₄ arylene, C₇₋₁₅ aralkylene, heteroarylene, heterocyclylene, C₁₋₆ heteroalkylene-C₆₋₁₄ arylene, C₁₋₆ heteroalkylene-heterocyclylene, or C₂₋₆ alkynylene-heterocyclylene; and wherein each alkylene, heteroalkylene, alkenylene, heteroalkenylene, alkynylene, heteroalkynylene, cycloalkylene, arylene, aralkylene, heteroarylene, and heterocyclylene is optionally substituted with one or more substituents Q.

128. The compound of any one of claims 1 to 74 and 112, wherein R^{E} is a moiety having the structure of: 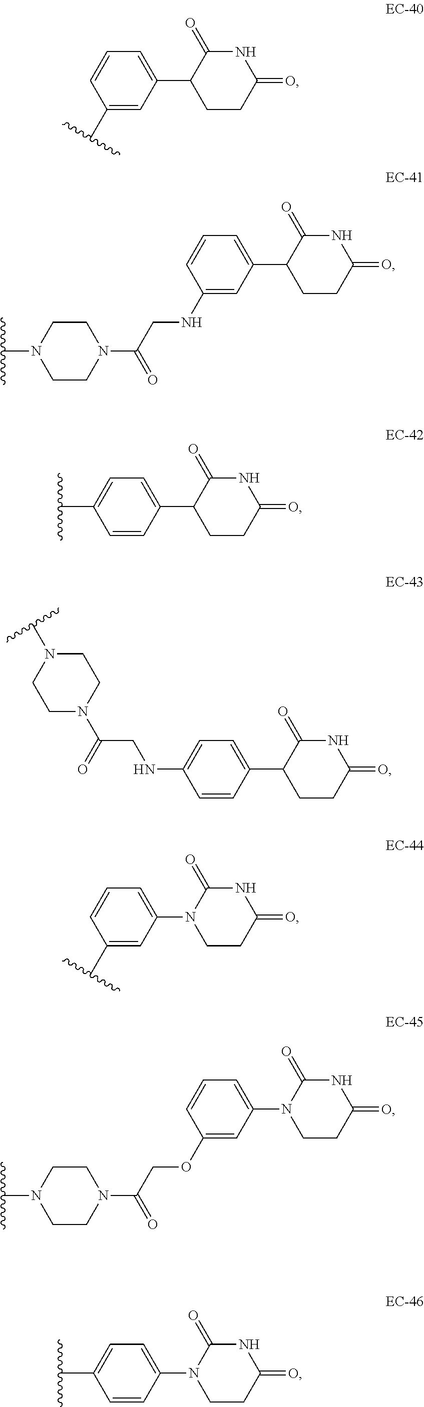 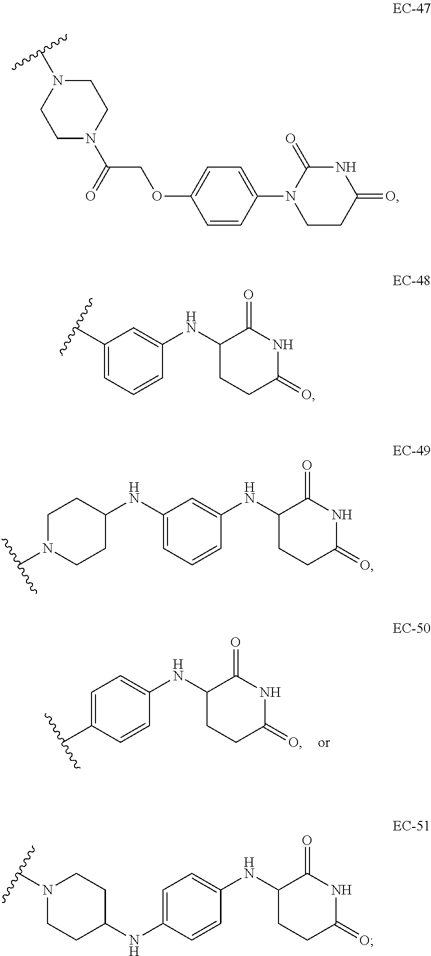 [SMILES: *N1CCN(C(=O)COc2ccc(N3CCC(=O)NC3=O)cc2)CC1 ; *c1cccc(NC2CCC(=O)NC2=O)c1 ; *N1CCC(Nc2cccc(NC3CCC(=O)NC3=O)c2)CC1 ; *c1ccc(NC2CCC(=O)NC2=O)cc1 ; *N1CCC(Nc2ccc(NC3CCC(=O)NC3=O)cc2)CC1]
or an enantiomer, a mixture of enantiomers, a tautomer, a mixture of two or more tautomers, or an isotopic variant thereof.

129. The compound of any one of claims 1 to 73, wherein R^{E} is a moiety of an IAP E3 ligand.

130. The compound of any one of claims 1 to 73 and 129, wherein R^{E} is a moiety having the structure of Formula (EI-I): 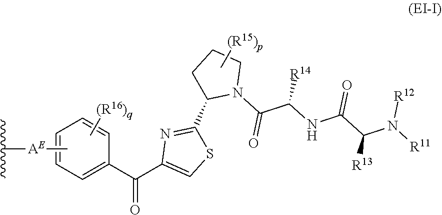
or an enantiomer, a mixture of enantiomers, a diastereomer, a mixture of two or more diastereomers, a tautomer, a mixture of two or more tautomers, or an isotopic variant thereof; wherein:
A^{E} is a bond, —O—, —N(R^{1b})—, —S—, C₁₋₆ alkylene, C₁₋₆ heteroalkylene, C₂₋₆ alkenylene, C₂₋₆ heteroalkenylene, C₂₋₆ alkynylene, C₂₋₆ heteroalkynylene, C₃₋₁₀ cycloalkylene, C₆₋₁₄ arylene, C₇₋₁₅ aralkylene, heteroarylene, heterocyclylene, C₁₋₆ heteroalkylene-C₆₋₁₄ arylene, C₁₋₆ heteroalkylene-heterocyclylene, or C₂₋₆ alkynylene-heterocyclylene;
R^{I1} and R^{I2} are each independently hydrogen, C₁₋₆ alkyl, or C₃₋₁₀ cycloalkyl;
R^{I3} and R^{I4} are each independently C₁₋₆ alkyl, C₃₋₁₀ cycloalkyl, or heterocyclyl;
each R^{I5} and R^{I6} is independently (i) deuterium, cyano, halo, nitro, or oxo; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
p is an integer of 0, 1, 2, 4, or 5; and
q is an integer of 0, 1, 2, or 4;
wherein each alkylene, heteroalkylene, alkenylene, heteroalkenylene, alkynylene, heteroalkynylene, cycloalkylene, arylene, aralkylene, heteroarylene, and heterocyclylene is optionally substituted with one or more substituents Q.

131. The compound of any one of claims 1 to 73 and 129, wherein R^{E} is a moiety having the structure of Formula (EI-IV): 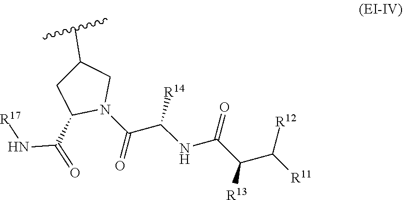
or an enantiomer, a mixture of enantiomers, a diastereomer, a mixture of two or more diastereomers, a tautomer, a mixture of two or more tautomers, or an isotopic variant thereof; wherein:
R^{I1} and R^{I2} are each independently hydrogen, C₁₋₆ alkyl, or C₃₋₁₀ cycloalkyl;
R^{I3} and R^{I4} are each independently C₁₋₆ alkyl, C₃₋₁₀ cycloalkyl, or heterocyclyl; and
R^{I7} is C₃₋₁₀ cycloalkyl, C₆₋₁₄ aryl, C₇₋₁₅ aralkyl, heteroaryl, or heterocyclyl;
wherein each alkyl, cycloalkyl, aryl, aralkyl, heteroaryl, and heterocyclyl is optionally substituted with one or more substituents Q.

132. The compound of any one of claims 1 to 73 and 129, wherein R^{E} is a moiety having the structure of Formula (EI-V): 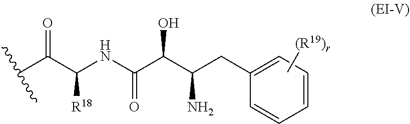
or an enantiomer, a mixture of enantiomers, a diastereomer, a mixture of two or more diastereomers, a tautomer, a mixture of two or more tautomers, or an isotopic variant thereof; wherein:
R⁸ is C₁₋₆ alkyl, C₃₋₁₀ cycloalkyl, or heterocyclyl;
each R¹⁹ is independently (i) deuterium, cyano, halo, nitro, or oxo; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; and
r is an integer of 0, 1, 2, 4, or 5;
wherein each alkyl, heteroalkyl, alkenyl, alkynyl, cycloalkyl, aryl, aralkyl, heteroaryl, and heterocyclyl is optionally substituted with one or more substituents Q.

133. The compound of any one of claims 1 to 73 and 129, wherein R^{E} is a moiety of an IAP E3 ligand having the structure of: 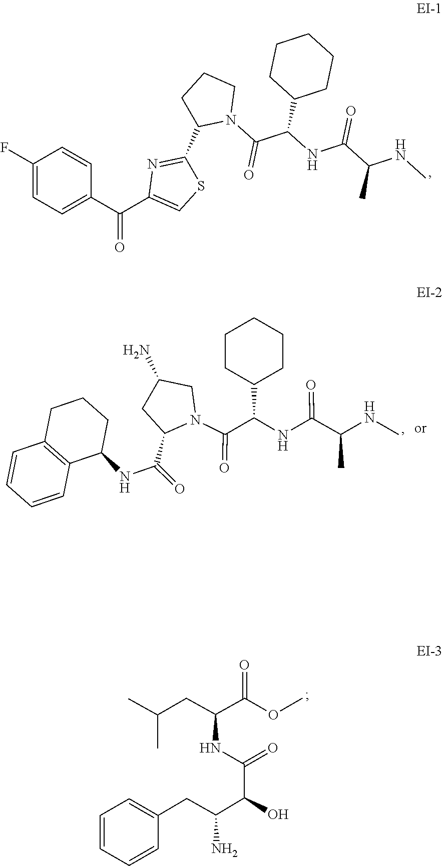
or a diastereomer, a mixture of two or more diastereomers, a tautomer, a mixture of two or more tautomers, or an isotopic variant thereof.

134. The compound of any one of claims 1 to 73, 129, and 133, wherein R^{E} is a moiety having the structure of: 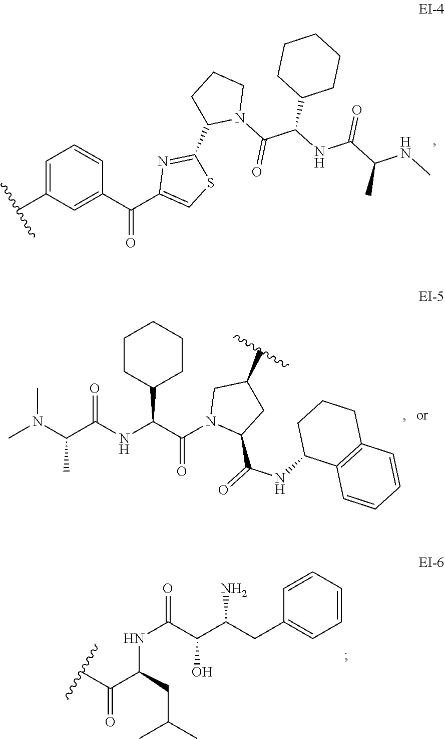
or a diastereomer, a mixture of two or more diastereomers, a tautomer, a mixture of two or more tautomers, or an isotopic variant thereof.

135. The compound of any one of claims 1 to 73, wherein R^{E} is a moiety of an MDM2 E3 ligand.

136. The compound of any one of claims 1 to 73 and 135, wherein R^{E} is a moiety of an MDM2 E3 ligand having the structure of Formula (EM-I): 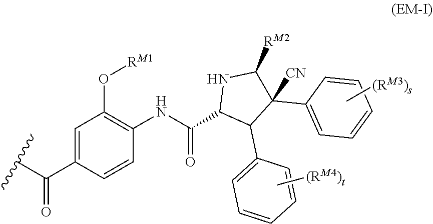
or an enantiomer, a mixture of enantiomers, a diastereomer, a mixture of two or more diastereomers, a tautomer, a mixture of two or more tautomers, or an isotopic variant thereof; wherein:
R^{M1} and R^{M2} are each independently C₁₋₆ alkyl or C₃₋₁₀ cycloalkyl;
each R^{M3} and R^{M4} is independently (i) deuterium, cyano, halo, nitro, or oxo; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NRda)NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; and
s and t are each independently an integer of 0, 1, 2, 3, 4, or 5;
wherein each alkyl, heteroalkyl, alkenyl, alkynyl, cycloalkyl, aryl, aralkyl, heteroaryl, and heterocyclyl is optionally substituted with one or more substituents Q.

137. The compound of any one of claims 1 to 73 and 135, wherein R^{E} is a moiety of an MDM2 E3 ligand having the structure of Formula (EM-III): 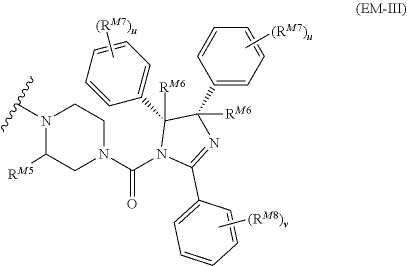
or an enantiomer, a mixture of enantiomers, a diastereomer, a mixture of two or more diastereomers, a tautomer, a mixture of two or more tautomers, or an isotopic variant thereof; wherein:
R^{M5} is hydrogen or oxo;
each R^{M6} is independently hydrogen, deuterium, or C₁₋₆ alkyl; and
each R^{M7} and R^{MS} is independently (i) deuterium, cyano, halo, nitro, or oxo; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; and
each u and v is independently an integer of 0, 1, 2, 3, 4, or 5;
wherein each alkyl, heteroalkyl, alkenyl, alkynyl, cycloalkyl, aryl, aralkyl, heteroaryl, and heterocyclyl is optionally substituted with one or more substituents Q.

138. The compound of any one of claims 1 to 73 and 135, wherein R^{E} is a moiety of an MDM2 E3 ligand having the structure of: 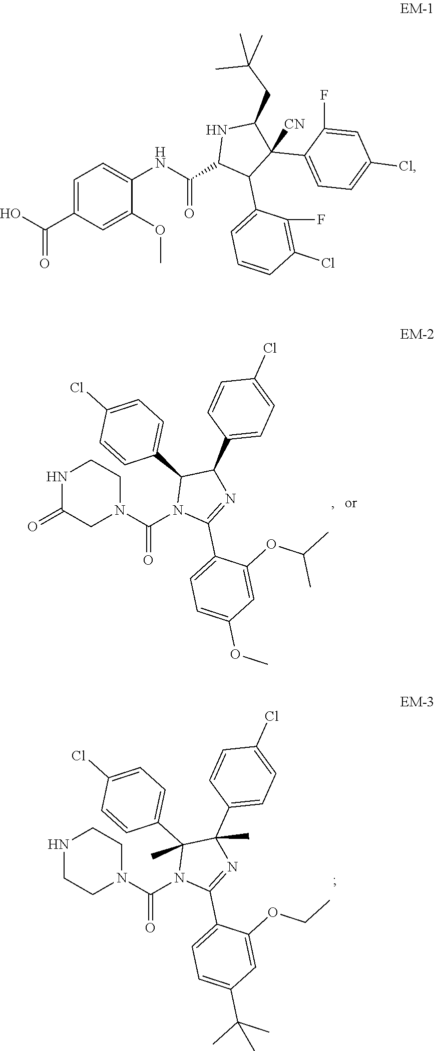
or a diastereomer, a mixture of two or more diastereomers, a tautomer, a mixture of two or more tautomers, or an isotopic variant thereof.

139. The compound of any one of claims 1 to 73, 135, and 138, wherein R^{E} is a moiety having the structure of: 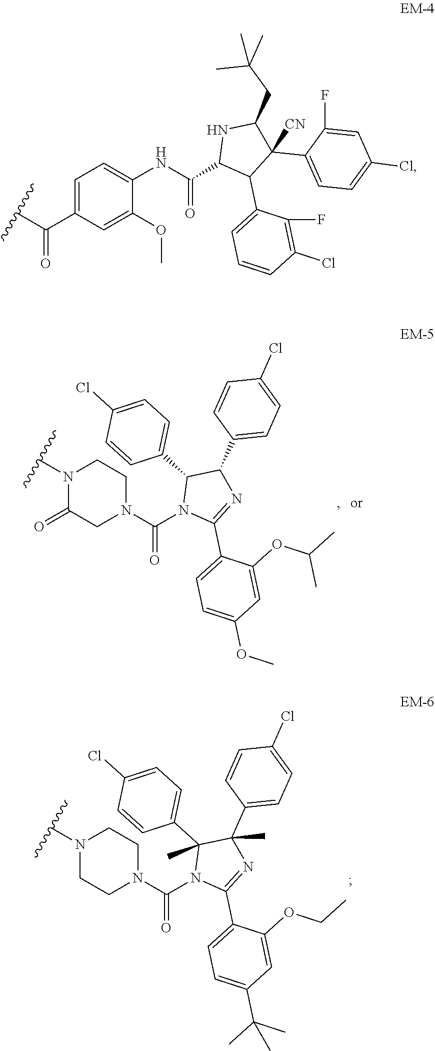
or a diastereomer, a mixture of two or more diastereomers, a tautomer, a mixture of two or more tautomers, or an isotopic variant thereof.

140. The compound of any one of claims 1 to 73, wherein R^{E} is a moiety of a VHL E3 ligand.

141. The compound of any one of claims 1 to 73 and 140, wherein R^{E} has the structure of Formula (EV-I): 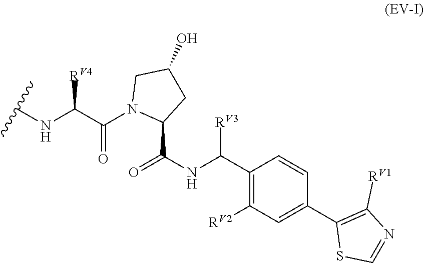
or a diastereomer, a mixture of two or more diastereomers, a tautomer, a mixture of two or more tautomers, or an isotopic variant thereof; wherein:
R^{V1}, R^{V3}, and R^{V4} are each independently hydrogen, deuterium, C₁₋₆ alkyl, or C₃₋₁₀ cycloalkyl; and
R^{V2} is hydrogen, deuterium, halo, hydroxyl, —OC₁₋₆ alkyl, or —OC₃₋₁₀ cycloalkyl;
wherein each alkyl and cycloalkyl is optionally substituted with one or more, in one embodiment, one, two, three, or four, substituents Q.

142. The compound of any one of claims 1 to 73 and 140, wherein R^{E} has the structure of Formula (EV-II): 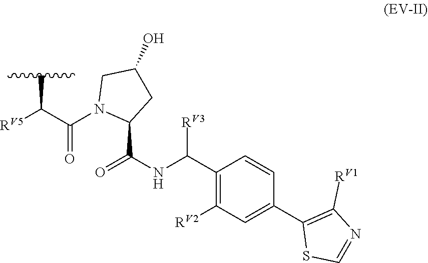
or a diastereomer, a mixture of two or more diastereomers, a tautomer, a mixture of two or more tautomers, or an isotopic variant thereof; wherein:
R^{V1} and R^{V3} are each independently hydrogen, deuterium, C₁₋₆ alkyl, or C₃₋₁₀ cycloalkyl;
R^{V2} is hydrogen, deuterium, halo, hydroxyl, —OC₁₋₆ alkyl, or —OC₃₋₁₀ cycloalkyl; and
R^{V5} is —NHC(O)C₁₋₆ alkyl, —NHC(O)C₃₋₁₀ cycloalkyl, or heterocyclyl;
wherein each alkyl, cycloalkyl, and heterocyclyl is optionally substituted with one or more, in one embodiment, one, two, three, or four, substituents Q.

143. The compound of any one of claims 1 to 73 and 140, wherein R^{E} has the structure of Formula (EV-III): 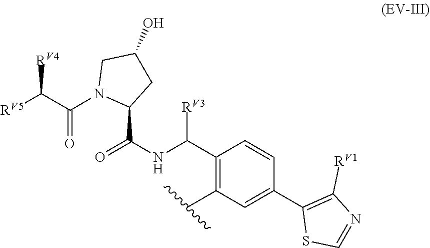
or a diastereomer, a mixture of two or more diastereomers, a tautomer, a mixture of two or more tautomers, or an isotopic variant thereof; wherein:
R^{V1}, R^{V3}, and R^{V4} are each independently hydrogen, deuterium, C₁₋₆ alkyl, or C₃₋₁₀ cycloalkyl; and
R^{V5} is —NHC(O)C₁₋₆ alkyl, —NHC(O)C₃₋₁₀ cycloalkyl, or heterocyclyl;
wherein each alkyl, cycloalkyl, and heterocyclyl is optionally substituted with one or more, in one embodiment, one, two, three, or four, substituents Q.

144. The compound of any one of claims 141 to 143, wherein R^{V1} is methyl.

145. The compound of claim 141, 142, or 144, wherein R^{V2} is hydrogen.

146. The compound of any one of claims 141 to 145, wherein R^{V3} is hydrogen.

147. The compound of any one of claims 141 and 143 to 146, wherein R^{V4} is propyl, butyl, or cyclopropyl, each of which is optionally substituted with cyano, fluoro, or trifluoromethyl.

148. The compound of any one of claims 141 and 143 to 147, wherein R^{V4} is isopropyl, tert-butyl, cyclopropyl, 1-fluorocyclopropyl, or 1-trifuloromethylcyclopropyl.

149. The compound of any one of claims 142 to 148, wherein R^{V5} is acetamido, cyclopropamido, or isoindolinyl, each of which is optionally substituted with cyano, fluoro, or trifluoromethyl.

150. The compound of any one of claims 142 to 149, wherein R^{V5} is acetamido, cyclopropamido, 1-cyanocyclopropamido, 1-fluorocyclopropamido, or 1-oxoisoindolin-2-yl.

151. The compound of any one of claims 1 to 73 and 140, wherein R^{E} is a moiety of a VHL E3 ligand having the structure of: 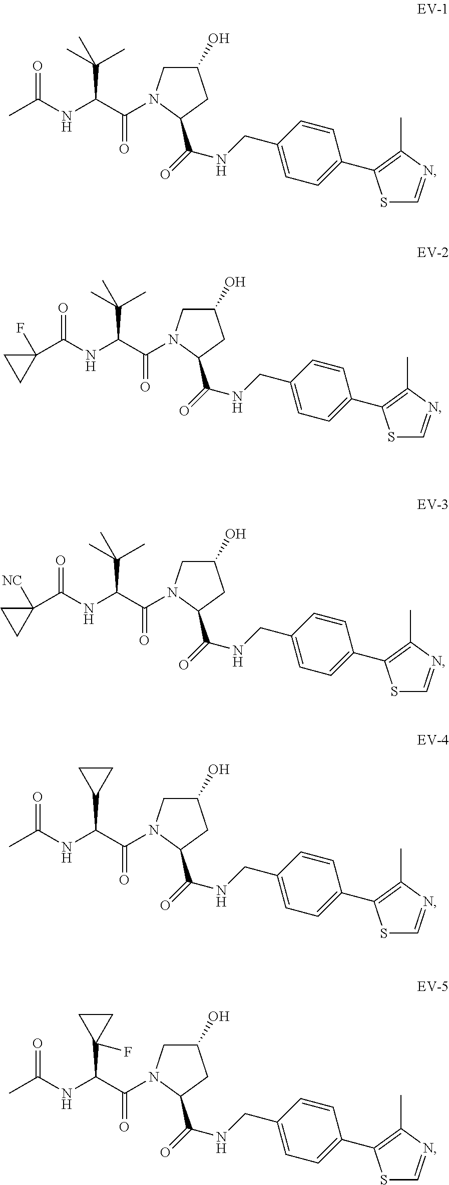 [SMILES: CC(=O)N[C@H](C(=O)N1C[C@H](O)C[C@H]1C(=O)NCc1ccc(-c2scnc2C)cc1)C(C)(C)C ; Cc1ncsc1-c1ccc(CNC(=O)[C@@H]2C[C@@H](O)CN2C(=O)[C@@H](NC(=O)C2(F)CC2)C(C)(C)C)cc1 ; Cc1ncsc1-c1ccc(CNC(=O)[C@@H]2C[C@@H](O)CN2C(=O)[C@@H](NC(=O)C2(C#N)CC2)C(C)(C)C)cc1 ; CC(=O)N[C@H](C(=O)N1C[C@H](O)C[C@H]1C(=O)NCc1ccc(-c2scnc2C)cc1)C1CC1 ; CC(=O)N[C@H](C(=O)N1C[C@H](O)C[C@H]1C(=O)NCc1ccc(-c2scnc2C)cc1)C1(F)CC1] 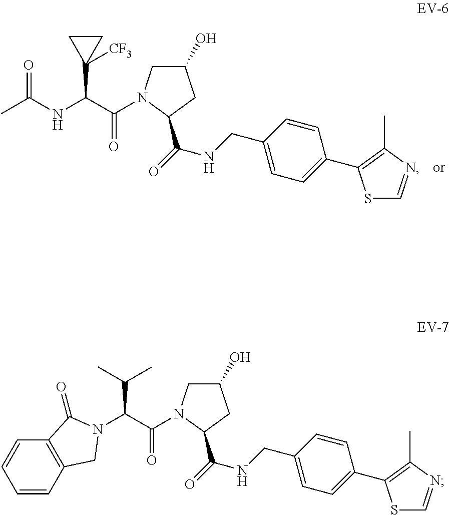 [SMILES: CC(=O)N[C@H](C(=O)N1C[C@H](O)C[C@H]1C(=O)NCc1ccc(-c2scnc2C)cc1)C1(C(F)(F)F)CC1 ; Cc1ncsc1-c1ccc(CNC(=O)[C@@H]2C[C@@H](O)CN2C(=O)[C@H](C(C)C)N2Cc3ccccc3C2=O)cc1]
or a diastereomer, a mixture of two or more diastereomers, a tautomer, a mixture of two or more tautomers, or an isotopic variant thereof.

152. The compound of any one of claims 1 to 73, 140, and 151, wherein R^{E} is a moiety having the structure of: 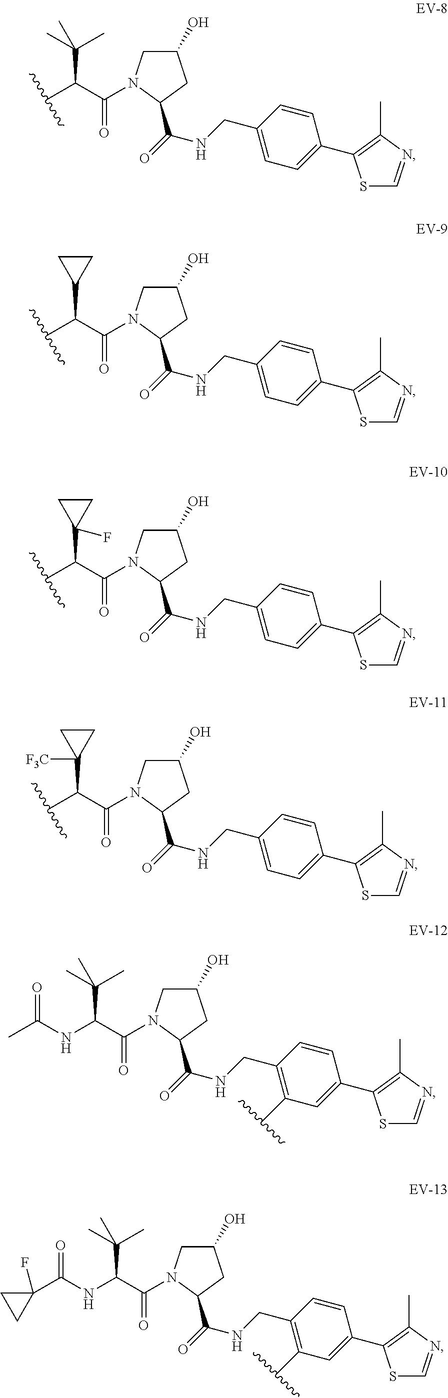 [SMILES: *[C@H](C(=O)N1C[C@H](O)C[C@H]1C(=O)NCc1ccc(-c2scnc2C)cc1)C(C)(C)C ; *[C@H](C(=O)N1C[C@H](O)C[C@H]1C(=O)NCc1ccc(-c2scnc2C)cc1)C1CC1 ; *[C@H](C(=O)N1C[C@H](O)C[C@H]1C(=O)NCc1ccc(-c2scnc2C)cc1)C1(F)CC1 ; *[C@H](C(=O)N1C[C@H](O)C[C@H]1C(=O)NCc1ccc(-c2scnc2C)cc1)C1(C(F)(F)F)CC1 ; *c1cc(-c2scnc2C)ccc1CNC(=O)[C@@H]1C[C@@H](O)CN1C(=O)[C@@H](NC(C)=O)C(C)(C)C ; *c1cc(-c2scnc2C)ccc1CNC(=O)[C@@H]1C[C@@H](O)CN1C(=O)[C@@H](NC(=O)C1(F)CC1)C(C)(C)C] 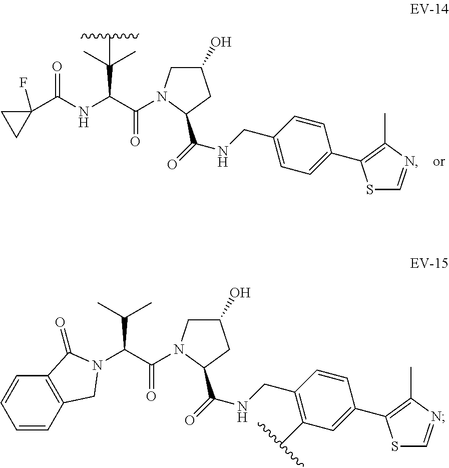 [SMILES: *C(C)(C)[C@H](NC(=O)C1(F)CC1)C(=O)N1C[C@H](O)C[C@H]1C(=O)NCc1ccc(-c2scnc2C)cc1 ; *c1cc(-c2scnc2C)ccc1CNC(=O)[C@@H]1C[C@@H](O)CN1C(=O)[C@H](C(C)C)N1Cc2ccccc2C1=O]
or a diastereomer, a mixture of two or more diastereomers, a tautomer, a mixture of two or more tautomers, or an isotopic variant thereof.

153. The compound of any one of claims 1 to 73, 140, 151, and 152, wherein R^{E} is a moiety having the structure of: 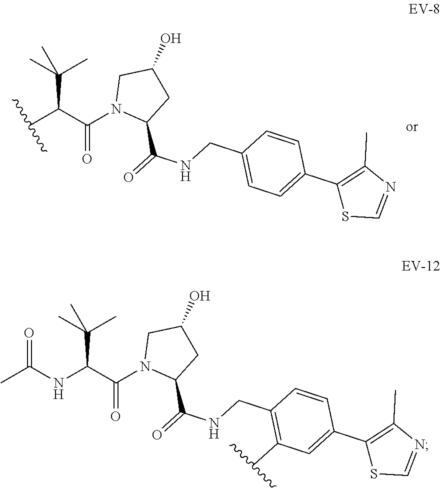 [SMILES: *[C@H](C(=O)N1C[C@H](O)C[C@H]1C(=O)NCc1ccc(-c2scnc2C)cc1)C(C)(C)C ; *c1cc(-c2scnc2C)ccc1CNC(=O)[C@@H]1C[C@@H](O)CN1C(=O)[C@@H](NC(C)=O)C(C)(C)C]
or a diastereomer, a mixture of two or more diastereomers, a tautomer, a mixture of two or more tautomers, or an isotopic variant thereof.

154. The compound of claim 79, wherein the compound has the structure of Formula (XVII): 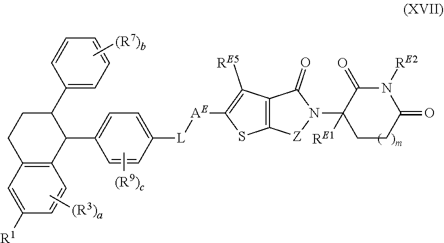
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

155. The compound of claim 79, wherein the compound has the structure of Formula (XVIII): 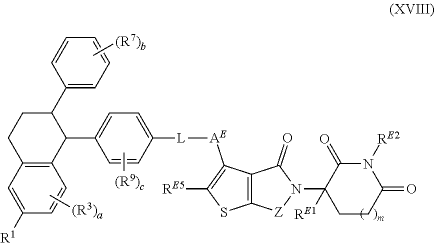
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

156. The compound of claim 80, wherein the compound has the structure of Formula (XIX): 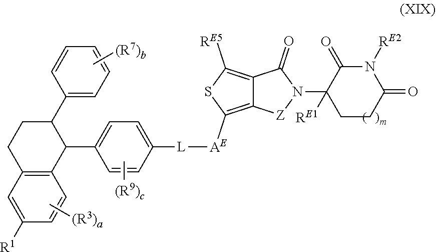
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

157. The compound of claim 80, wherein the compound has the structure of Formula (XX): 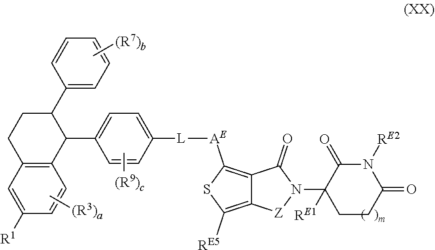
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

158. The compound of claim 99, wherein the compound has the structure of Formula 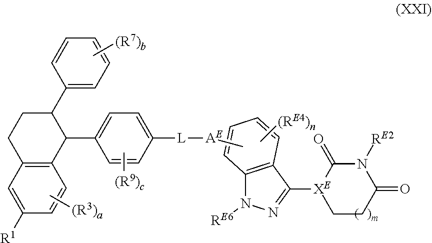
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

159. The compound of claim 99 or 158, wherein the compound has the structure of Formula (XXII): 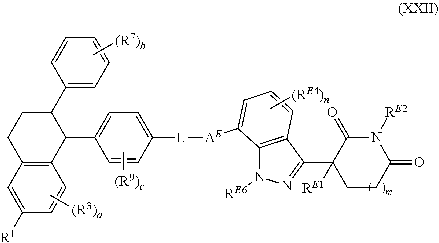
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

160. The compound of claim 99 or 158, wherein the compound has the structure of Formula (XXIII): 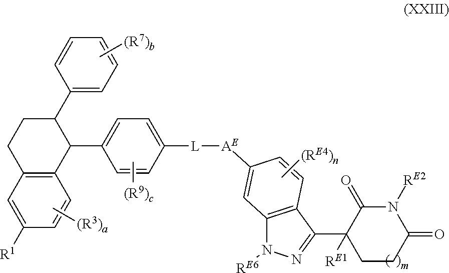
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

161. The compound of claim 112, wherein the compound has the structure of Formula (XXIV): 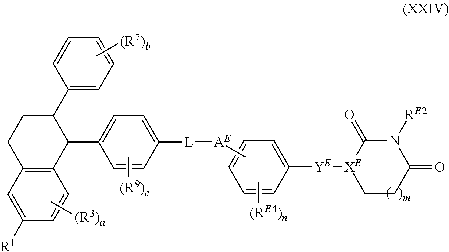
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

162. The compound of claim 112 or 161, wherein the compound has the structure of Formula (XXV): 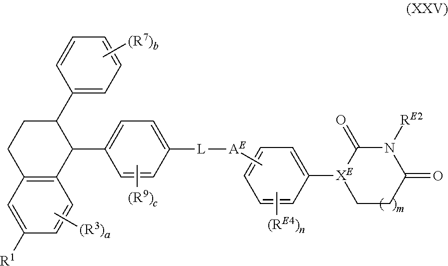
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

163. The compound of claim 112, 161, or 162, wherein the compound has the structure of Formula (XXVI): 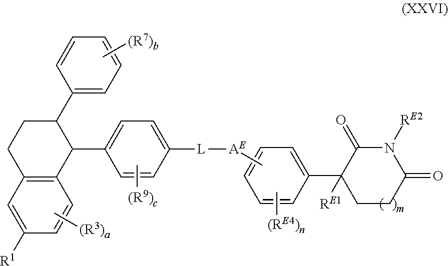
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

164. The compound of any one of claims 112 and 161 to 163, wherein the compound has the structure of Formula (XXVII): 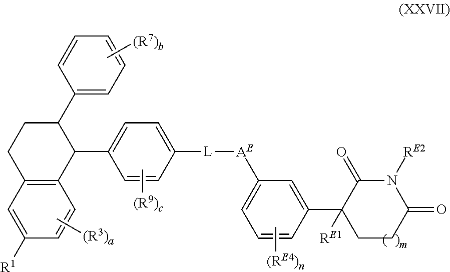
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

165. The compound of any one of claims 112 and 161 to 163, wherein the compound has the structure of Formula (XXVIII): 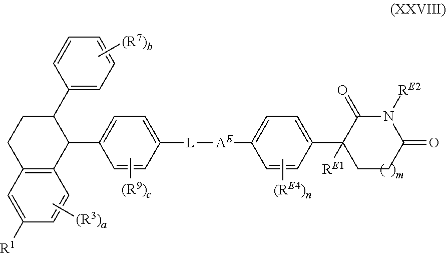
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

166. The compound of claim 112, 161, or 162, wherein the compound has the structure of Formula (XXIX): 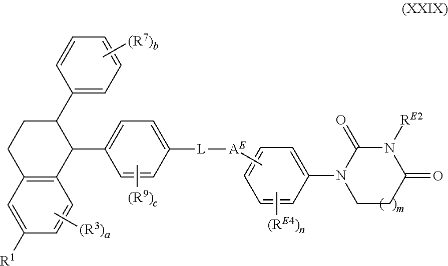
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

167. The compound of claim 112, 161, 162, or 166, wherein the compound has the structure of Formula (XXX): 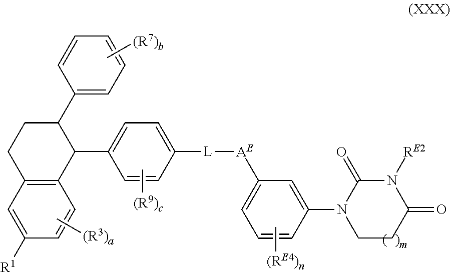
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

168. The compound of claim 112, 161, 162, or 166, wherein the compound has the structure of Formula (XXXI): 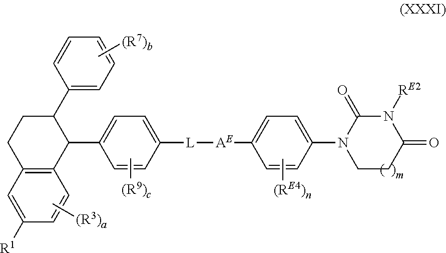
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

169. The compound of claim 112 or 161, wherein the compound has the structure of Formula (XXXII): 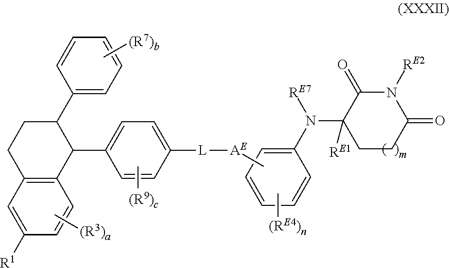
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

170. The compound of claim 112, 161, or 169, wherein the compound has the structure of Formula (XXXIII): 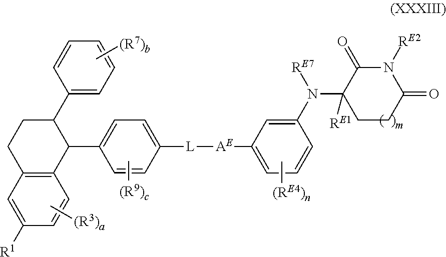
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

171. The compound of claim 112, 161, or 169, wherein the compound has the structure of Formula (XXXIV): 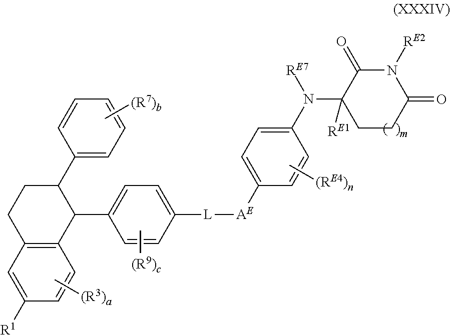
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

172. The compound of claim 79, wherein the compound has the structure of Formula (XXXIX): 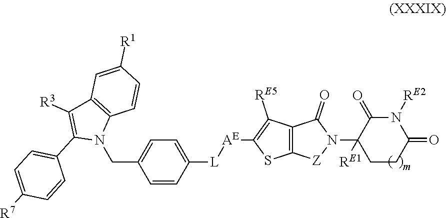
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

173. The compound of claim 79, wherein the compound has the structure of Formula 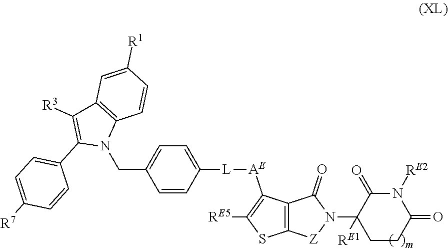
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

174. The compound of claim 80, wherein the compound has the structure of Formula (XLI): 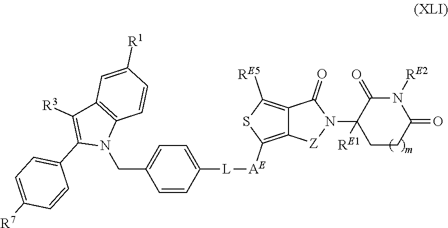
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

175. The compound of claim 80, wherein the compound has the structure of Formula (XLII): 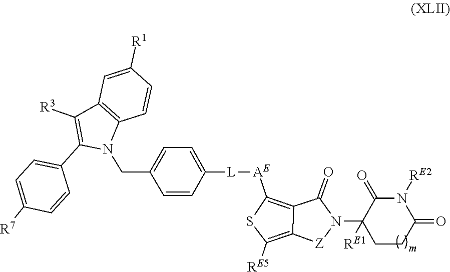
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

176. The compound of claim 99, wherein the compound has the structure of Formula (XLIII): 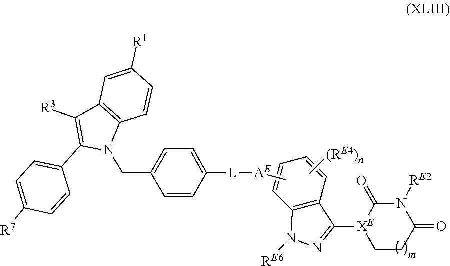
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

177. The compound of claim 99 or 176, wherein the compound has the structure of Formula (XLIV): 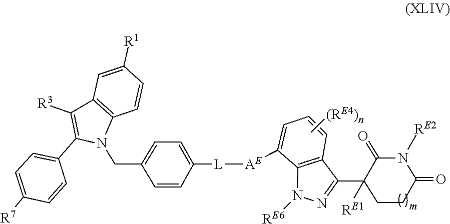
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

178. The compound of claim 99 or 176, wherein the compound has the structure of Formula (XLV): 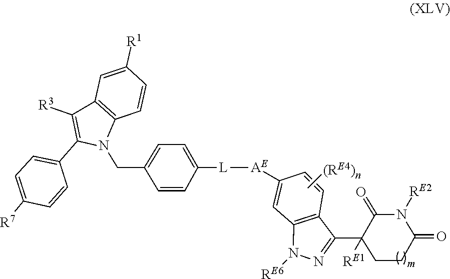
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

179. The compound of claim 112, wherein the compound has the structure of Formula (XLVI): 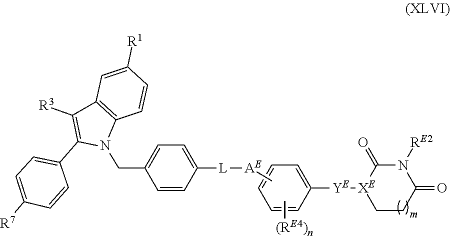
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

180. The compound of claim 112 or 179, wherein the compound has the structure of Formula (XLVII): 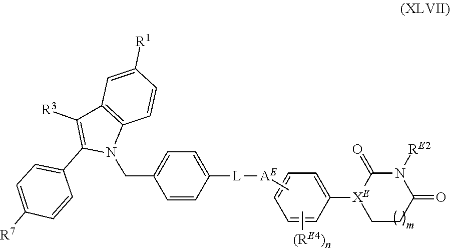
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

181. The compound of claim 112, 179, or 180, wherein the compound has the structure of Formula (XLVIII): 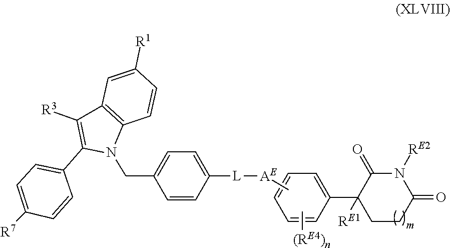
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

182. The compound of any one of claims 112 and 179 to 181, wherein the compound has the structure of Formula (XLIX): 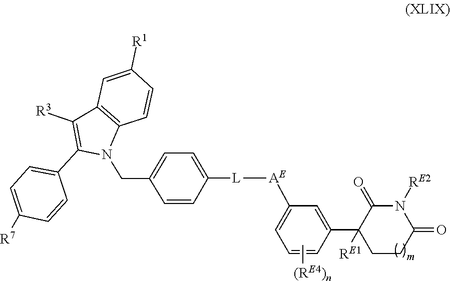
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

183. The compound of any one of claims 112 and 179 to 181, wherein the compound has the structure of Formula (L): 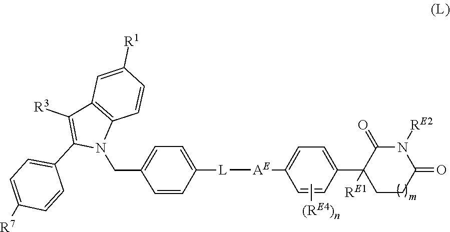
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

184. The compound of claim 112, 179, or 180, wherein the compound has the structure of Formula (LI): 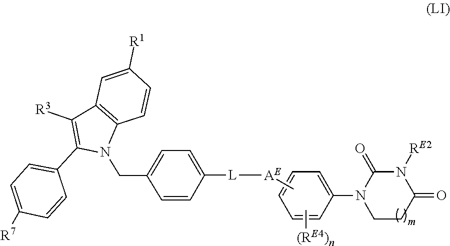
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

185. The compound of claim 112, 179, 180, or 184, wherein the compound has the structure of Formula (LII): 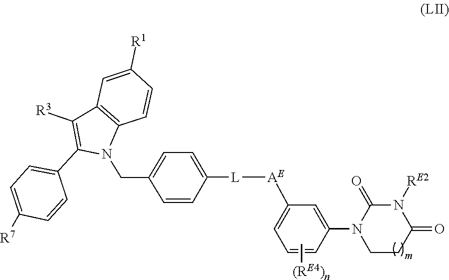
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

186. The compound of claim 112, 179, 180, or 184, wherein the compound has the structure of Formula (LIII): 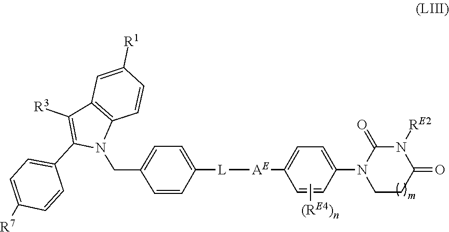
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

187. The compound of claim 112 or 179, wherein the compound has the structure of Formula (LIV): 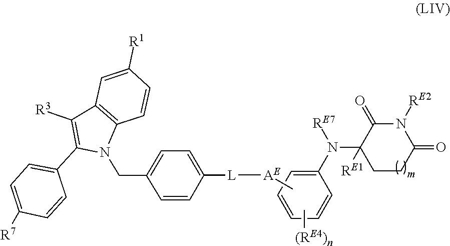
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

188. The compound of claim 112, 179, or 187, wherein the compound has the structure of Formula (LV): 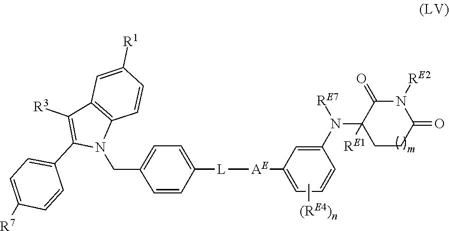
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

189. The compound of claim 112, 179, or 187, wherein the compound has the structure of Formula (LVI): 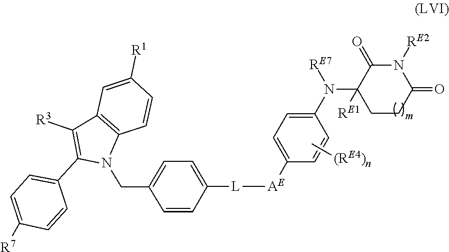
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

190. The compound of any one of claims 1 to 189, wherein L has the structure of:
—Z^{L}—(R^{L}—Z^{L})_{z}—;
wherein:
each R^{L} is independently C₁₋₁₀ alkylene, C₂₋₁₀ alkenylene, C₂₋₁₀ alkynylene, C₃₋₁₀ cycloalkylene, C₆₋₁₄ arylene, heteroarylene, or heterocyclylene, each of which is optionally substituted with one or more substituents Q;
each Z^{L} is independently a bond, —C(O)—, —C(O)O—, —C(O)NR^{1b}—, —C(O)S—, —C(NR^{1a})NR^{1b}—, —C(S)—, —C(S)O—, —C(S)NR^{1b}—, —O—, —OC(O)O—, —OC(O)NR^{1b}—, —OC(O)S—, —OC(NR^{1a})NR^{1b}—, —OC(S)—, —OC(S)O—, —OC(S)NR^{1b}—, —OS(O)—, —OS(O)₂—, —OS(O)NR^{1b}—, —OS(O)₂NR^{1b}—, —NR^{1b}—, —NR^{1a}C(O)NR^{1b}—, —NR^{1a}C(O)S—, —NR^{1a}C(NR^{1d})NR^{1b}—, —NR^{1a}C(S)NR^{1b}—, —NR^{1a}S(O)NR^{1b}—, —NR^{1a}S(O)₂NR^{1b}—, —S—, —S(O)—, —S(O)₂—, —S(O)NR^{1b}—, or —S(O)₂NR^{1b}—; and
z is an integer of 1, 2, 3, 4, 5, 6, 7, 8, 9, or 10.

191. The compound of claim 190, wherein each R^{L} is independently C₁₋₁₀ alkylene, C₂₋₁₀ alkynylene, C₃₋₁₀ cycloalkylene, C₆₋₁₄ arylene, heteroarylene, or heterocyclylene, each of which is optionally substituted with one or more substituents Q.

192. The compound of claim 190 or 191, wherein each R^{L} is independently methanediyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, ethyne-1,2-diyl, cyclobutane-1,3-diyl, cyclopentane-1,3-diyl, cyclohexane-1,3-diyl, cyclohexane-1,4-diyl, cycloheptane-1,3-diyl, cycloheptane-1,4-diyl, bicyclo[2.2.2]octane-1,4-diyl, phen-1,3-diyl, phen-1,4-diyl, pyrazol-1,3-diyl, pyrazol-1,4-diyl, imidazol-1,4-diyl, 1,2,3-triazol-1,4-diyl, pyrimidin-2,4-diyl, pyrimidin-2,5-diyl, 5,6,7,8,9,10-hexahydrocycloocta[d]-pyridazin-1,7-diyl, pyrazolidin-1,3-diyl, pyrazolidin-1,4-diyl, 1,3-dioxan-2,5-diyl, piperazin-1,4-diyl, piperidin-1,3-diyl, piperidin-1,4-diyl, or 3,9-diazaspiro[5.5]-undecane-3,9-diyl, each of which is optionally substituted with one or more substituents Q.

193. The compound of any one of claims 190 to 192, wherein each Z^{L} is independently a bond, —C(O)—, —C(O)NR^{1b}—, —O—, —OC(O)NR^{1b}—, —NR^{1b}—, or —NR^{1a}C(O)NR^{1b}—.

194. The compound of any one of claims 190 to 193, wherein each Z^{L} is independently a bond, —C(O)—, —C(O)O—, —C(O)NH—, —OC(O)NH—, —O—, —NH—, —N(CH₃)—, or —NHC(O)NH—.

195. The compound of any one of claims 190 to 194, wherein z is an integer of 1, 2, 3, or 4.

196. A compound of:
3-(6-fluoro-4-(1-(4-(4-((1R,2S)-6-hydroxy-2-phenyl-1,2,3,4-tetrahydro-naphthalen-1-yl)phenoxy)butyl)piperidin-4-yl)-1-oxoisoindolin-2-yl)piperidine-2,6-dione A1;
3-(6-fluoro-4-(1-(6-(4-((1R,2S)-6-hydroxy-2-phenyl-1,2,3,4-tetrahydro-naphthalen-1-yl)phenoxy)hexyl)piperidin-4-yl)-1-oxoisoindolin-2-yl)piperidine-2,6-dione A2;
3-(6-fluoro-4-(1-((1-(4-((1R,2S)-6-hydroxy-2-phenyl-1,2,3,4-tetrahydro-naphthalen-1-yl)phenyl)piperidin-4-yl)methyl)piperidin-4-yl)-1-oxoisoindolin-2-yl)piperidine-2,6-dione A3;
3-(6-fluoro-5-(1-(4-(4-((1R,2S)-6-hydroxy-2-phenyl-1,2,3,4-tetrahydro-naphthalen-1-yl)phenoxy)butyl)piperidin-4-yl)-1-oxoisoindolin-2-yl)piperidine-2,6-dione A4;
3-(6-fluoro-5-(1-(6-(4-((1R,2S)-6-hydroxy-2-phenyl-1,2,3,4-tetrahydro-naphthalen-1-yl)phenoxy)hexyl)piperidin-4-yl)-1-oxoisoindolin-2-yl)piperidine-2,6-dione A5;
3-(6-fluoro-5-(1-((1-(4-((1R,2S)-6-hydroxy-2-phenyl-1,2,3,4-tetrahydro-naphthalen-1-yl)phenyl)piperidin-4-yl)methyl)piperidin-4-yl)-1-oxoisoindolin-2-yl)piperidine-2,6-dione A6;
3-(1-(1-((1-(4-((1R,2S)-6-hydroxy-2-phenyl-1,2,3,4-tetrahydronaphthalen-1-yl)phenyl)piperidin-4-yl)methyl)piperidin-4-yl)-4-oxo-4H-thieno[3,4-c]pyrrol-5(6H)-yl)-piperidine-2,6-dione A7;
3-(1-(1-(4-(4-((1R,2S)-6-hydroxy-2-phenyl-1,2,3,4-tetrahydronaphthalen-1-yl)phenoxy)butyl)piperidin-4-yl)-4-oxo-4H-thieno[3,4-c]pyrrol-5(6H)-yl)piperidine-2,6-dione A8;
3-(1-((4-(((5-(4-((1R,2S)-6-hydroxy-2-phenyl-1,2,3,4-tetrahydronaphthalen-1-yl)phenoxy)pentyl)amino)methyl)phenoxy)methyl)-4-oxo-4H-thieno[3,4-c]pyrrol-5(6H)-yl)piperidine-2,6-dione A9;
3-(2-(1-((1-(4-((1R,2S)-6-hydroxy-2-phenyl-1,2,3,4-tetrahydronaphthalen-1-yl)phenyl)piperidin-4-yl)methyl)piperidin-4-yl)-4-oxo-4,6-dihydro-5H-thieno[2,3-c]pyrrol-5-yl)piperidine-2,6-dione A10;
3-(2-(1-(4-(4-((1R,2S)-6-hydroxy-2-phenyl-1,2,3,4-tetrahydronaphthalen-1-yl)phenoxy)butyl)piperidin-4-yl)-4-oxo-4,6-dihydro-5H-thieno[2,3-c]pyrrol-5-yl)piperidine-2,6-dione A11;
3-(6-fluoro-4-(1-(4-(4-((1S,2R)-6-hydroxy-2-phenyl-1,2,3,4-tetrahydro-naphthalen-1-yl)phenoxy)butyl)piperidin-4-yl)-1-oxoisoindolin-2-yl)piperidine-2,6-dione A12;
3-(6-fluoro-5-(1-(4-(4-((1S,2R)-6-hydroxy-2-phenyl-1,2,3,4-tetrahydro-naphthalen-1-yl)phenoxy)butyl)piperidin-4-yl)-1-oxoisoindolin-2-yl)piperidine-2,6-dione A13;
3-(5-(4-((1-(4-((1R,2S)-6-hydroxy-2-phenyl-1,2,3,4-tetrahydronaphthalen-1-yl)-phenyl)piperidin-4-yl)methyl)piperazin-1-yl)-1-oxoisoindolin-2-yl)piperidine-2,6-dione A14;
(R)-3-(5-(4-((1-(4-((1R,2S)-6-hydroxy-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione A15;
(S)-3-(5-(4-((1-(4-((1R,2S)-6-hydroxy-2-phenyl-1,2,3,4-tetrahydronaphthalen-1-yl)phenyl)piperidin-4-yl)methyl)piperazin-1-yl)-1-oxoisoindolin-2-yl)piperidine-2,6-dione A16;
3-(1-((4-(((5-(4-((1R,2S)-6-hydroxy-2-phenyl-1,2,3,4-tetrahydronaphthalen-1-yl)phenoxy)pentyl)amino)methyl)phenoxy)methyl)-4-oxo-4H-thieno[3,4-c]pyrrol-5(6H)-yl)piperidine-2,6-dione A17;
3-(1-(1-(6-(4-((1R,2S)-6-hydroxy-2-phenyl-1,2,3,4-tetrahydronaphthalen-1-yl)-phenoxy)hexyl)piperidin-4-yl)-4-oxo-4H-thieno[3,4-c]pyrrol-5(6H)-yl)piperidine-2,6-dione A18;
3-(1-(1-(4-(4-((1R,2S)-6-hydroxy-2-phenyl-1,2,3,4-tetrahydronaphthalen-1-yl)-phenoxy)butyl)piperidin-4-yl)-4-oxo-4H-thieno[3,4-c]pyrrol-5(6H)-yl)piperidine-2,6-dione A19;
3-(2-(1-(4-(4-((1R,2S)-6-hydroxy-2-phenyl-1,2,3,4-tetrahydronaphthalen-1-yl)-phenoxy)butyl)piperidin-4-yl)-4-oxo-4,6-dihydro-5H-thieno[2,3-c]pyrrol-5-yl)piperidine-2,6-dione A20;
3-(2-(1-(6-(4-((1R,2S)-6-hydroxy-2-phenyl-1,2,3,4-tetrahydronaphthalen-1-yl)-phenoxy)hexyl)piperidin-4-yl)-4-oxo-4,6-dihydro-5H-thieno[2,3-c]pyrrol-5-yl)piperidine-2,6-dione A21;
3-(7-(2-(4-(4-(4-((1R,2S)-6-hydroxy-2-phenyl-1,2,3,4-tetrahydronaphthalen-1-yl)phenoxy)butyl)piperazin-1-yl)-2-oxoethoxy)-1-methyl-1H-indazol-3-yl)piperidine-2,6-dione B1;
3-(6-(2-(4-(4-(4-((1R,2S)-6-hydroxy-2-phenyl-1,2,3,4-tetrahydronaphthalen-1-yl)phenoxy)butyl)piperazin-1-yl)-2-oxoethoxy)-1-methyl-1H-indazol-3-yl)piperidine-2,6-dione B2;
3-(7-((4-(((4-(4-((1R,2S)-6-hydroxy-2-phenyl-1,2,3,4-tetrahydronaphthalen-1-yl)phenoxy)butyl)amino)methyl)benzyl)oxy)-1-methyl-1H-indazol-3-yl)piperidine-2,6-dione B3;
3-(6-((4-(((4-(4-((1S,2R)-6-hydroxy-2-phenyl-1,2,3,4-tetrahydronaphthalen-1-yl)phenoxy)butyl)amino)methyl)benzyl)oxy)-1-methyl-1H-indazol-3-yl)piperidine-2,6-dione B4;
3-(3-(2-(4-(4-(4-((1R,2S)-6-hydroxy-2-phenyl-1,2,3,4-tetrahydronaphthalen-1-yl)phenoxy)butyl)piperazin-1-yl)-2-oxoethoxy)phenyl)piperidine-2,6-dione B5;
3-(4-(2-(4-(4-(4-((1R,2S)-6-hydroxy-2-phenyl-1,2,3,4-tetrahydronaphthalen-1-yl)phenoxy)butyl)piperazin-1-yl)-2-oxoethoxy)phenyl)piperidine-2,6-dione B6;
3-(3-((4-(((4-(4-((1S,2R)-6-hydroxy-2-phenyl-1,2,3,4-tetrahydronaphthalen-1-yl)phenoxy)butyl)amino)methyl)benzyl)oxy)phenyl)piperidine-2,6-dione B7;
3-(4-((4-(((4-(4-((1S,2R)-6-hydroxy-2-phenyl-1,2,3,4-tetrahydronaphthalen-1-yl)phenoxy)butyl)amino)methyl)benzyl)oxy)phenyl)piperidine-2,6-dione B8;
1-(3-(2-(4-(4-(4-((1R,2S)-6-hydroxy-2-phenyl-1,2,3,4-tetrahydronaphthalen-1-yl)phenoxy)butyl)piperazin-1-yl)-2-oxoethoxy)phenyl)dihydropyrimidine-2,4(1H,3H)-dione B9;
1-(4-(2-(4-(4-(4-((1R,2S)-6-hydroxy-2-phenyl-1,2,3,4-tetrahydronaphthalen-1-yl)phenoxy)butyl)piperazin-1-yl)-2-oxoethoxy)phenyl)dihydropyrimidine-2,4(1H,3H)-dione B10;
1-(3-((4-(((4-(4-((1S,2R)-6-hydroxy-2-phenyl-1,2,3,4-tetrahydronaphthalen-1-yl)phenoxy)butyl)amino)methyl)benzyl)oxy)phenyl)dihydropyrimidine-2,4(1H,3H)-dione B11;
1-(4-((4-(((4-(4-((1S,2R)-6-hydroxy-2-phenyl-1,2,3,4-tetrahydronaphthalen-1-yl)phenoxy)butyl)amino)methyl)benzyl)oxy)phenyl)dihydropyrimidine-2,4(1H,3H)-dione B12;
3-((3-(4-((1-(4-((1R,2S)-6-hydroxy-2-phenyl-1,2,3,4-tetrahydronaphthalen-1-yl)phenyl)piperidin-4-yl)methyl)piperazin-1-yl)phenyl)amino)piperidine-2,6-dione B13;
3-((3-((1-((1-(4-((1R,2S)-6-hydroxy-2-phenyl-1,2,3,4-tetrahydronaphthalen-1-yl)phenyl)piperidin-4-yl)methyl)piperidin-4-yl)amino)phenyl)amino)piperidine-2,6-dione B14;
3-((3-((2-(4-((1-(4-((1R,2S)-6-hydroxy-2-phenyl-1,2,3,4-tetrahydronaphthalen-1-yl)phenyl)piperidin-4-yl)methyl)piperazin-1-yl)ethyl)amino)phenyl)amino)piperidine-2,6-dione B15;
3-((3-(2-(4-((1-(4-((1R,2S)-6-hydroxy-2-phenyl-1,2,3,4-tetrahydronaphthalen-1-yl)phenyl)piperidin-4-yl)methyl)piperazin-1-yl)-2-oxoethoxy)phenyl)amino)piperidine-2,6-dione B16;
3-((3-((4-(((4-(4-((1S,2R)-6-hydroxy-2-phenyl-1,2,3,4-tetrahydronaphthalen-1-yl)phenoxy)butyl)amino)methyl)benzyl)oxy)phenyl)amino)piperidine-2,6-dione B17;
3-((4-((4-(((4-(4-((1S,2R)-6-hydroxy-2-phenyl-1,2,3,4-tetrahydronaphthalen-1-yl)phenoxy)butyl)amino)methyl)benzyl)oxy)phenyl)amino)piperidine-2,6-dione B18;
N-(3-((2,6-dioxopiperidin-3-yl)amino)phenyl)-2-(4-((1-(4-((1R,2S)-6-hydroxy-2-phenyl-1,2,3,4-tetrahydronaphthalen-1-yl)phenyl)piperidin-4-yl)methyl)piperazin-1-yl)-acetamide B19;
N-(3-((2,6-dioxopiperidin-3-yl)amino)phenyl)-2-(4-((1-(4-((1R,2S)-6-hydroxy-2-phenyl-1,2,3,4-tetrahydronaphthalen-1-yl)phenyl)piperidin-4-yl)methyl)piperazin-1-yl)-N-methylacetamide B20;
3-((3-((2-(4-((1-(4-((1R,2S)-6-hydroxy-2-phenyl-1,2,3,4-tetrahydronaphthalen-1-yl)phenyl)piperidin-4-yl)methyl)piperazin-1-yl)-2-oxoethyl)amino)phenyl)amino)piperidine-2,6-dione B21;
3-((3-((2-(4-((1-(4-((1R,2S)-6-hydroxy-2-phenyl-1,2,3,4-tetrahydronaphthalen-1-yl)phenyl)piperidin-4-yl)methyl)piperazin-1-yl)-2-oxoethyl)(methyl)amino)phenyl)amino)-piperidine-2,6-dione B22;
(S)-3-(6-(2-(4-(4-(4-((1R,2S)-6-hydroxy-2-phenyl-1,2,3,4-tetrahydronaphthalen-1-yl)phenoxy)butyl)piperazin-1-yl)-2-oxoethoxy)-1-methyl-1H-indazol-3-yl)piperidine-2,6-dione B23;
3-(6-(2-(4-(4-(4-((1S,2R)-6-hydroxy-2-phenyl-1,2,3,4-tetrahydronaphthalen-1-yl)phenoxy)butyl)piperazin-1-yl)-2-oxoethoxy)-1-methyl-1H-indazol-3-yl)piperidine-2,6-dione B24;
3-(6-(4-((1-(4-((1R,2S)-6-hydroxy-2-phenyl-1,2,3,4-tetrahydronaphthalen-1-yl)-phenyl)piperidin-4-yl)methyl)piperazin-1-yl)-1-methyl-1H-indazol-3-yl)piperidine-2,6-dione B25;
(R)-3-(6-(2-(4-(4-(4-((1R,2S)-6-hydroxy-2-phenyl-1,2,3,4-tetrahydronaphthalen-1-yl)phenoxy)butyl)piperazin-1-yl)-2-oxoethoxy)-1-methyl-1H-indazol-3-yl)piperidine-2,6-dione B26;
3-(6-(2-(4-((1-(4-((1R,2S)-6-hydroxy-2-phenyl-1,2,3,4-tetrahydronaphthalen-1-yl)phenyl)piperidin-4-yl)methyl)piperazin-1-yl)-2-oxoethoxy)-1-methyl-1H-indazol-3-yl)-piperidine-2,6-dione B27;
3-(6-(2-(4-(3-(1-(4-((1R,2S)-6-hydroxy-2-phenyl-1,2,3,4-tetrahydronaphthalen-1-yl)phenyl)piperidin-4-yl)propyl)piperazin-1-yl)-2-oxoethoxy)-1-methyl-1H-indazol-3-yl)-piperidine-2,6-dione B28;
3-(6-(4-(2-(4-((1R,2S)-6-hydroxy-2-phenyl-1,2,3,4-tetrahydronaphthalen-1-yl)-phenoxy)ethyl)piperazin-1-yl)-1-methyl-1H-indazol-3-yl)piperidine-2,6-dione B29;
3-(6-((1-(4-(4-((1R,2S)-6-hydroxy-2-phenyl-1,2,3,4-tetrahydronaphthalen-1-yl)-phenoxy)butyl)piperidin-4-yl)oxy)-1-methyl-1H-indazol-3-yl)piperidine-2,6-dione B30;
3-(6-(4-(3-(1-(4-((1R,2S)-6-hydroxy-2-phenyl-1,2,3,4-tetrahydronaphthalen-1-yl)phenyl)piperidin-4-yl)propyl)piperazin-1-yl)-1-methyl-1H-indazol-3-yl)piperidine-2,6-dione B31;
3-(6-((1-(2-(4-((1R,2S)-6-hydroxy-2-phenyl-1,2,3,4-tetrahydronaphthalen-1-yl)-phenoxy)ethyl)piperidin-4-yl)oxy)-1-methyl-1H-indazol-3-yl)piperidine-2,6-dione B32;
3-(6-(2-(4-(2-(4-((1R,2S)-6-hydroxy-2-phenyl-1,2,3,4-tetrahydronaphthalen-1-yl)phenoxy)ethyl)piperazin-1-yl)-2-oxoethoxy)-1-methyl-1H-indazol-3-yl)piperidine-2,6-dione B33;
3-(6-(2-(4-(4-(4-((1R,2S)-6-hydroxy-2-phenyl-1,2,3,4-tetrahydronaphthalen-1-yl)phenoxy)butyl)piperidin-1-yl)-2-oxoethoxy)-1-methyl-1H-indazol-3-yl)piperidine-2,6-dione B34;
3-(6-((1-(4-(4-((1R,2S)-6-hydroxy-2-phenyl-1,2,3,4-tetrahydronaphthalen-1-yl)-phenoxy)butanoyl)piperidin-4-yl)oxy)-1-methyl-1H-indazol-3-yl)piperidine-2,6-dione B35;
3-(6-(2-(4-(2-(1-(4-((1R,2S)-6-hydroxy-2-phenyl-1,2,3,4-tetrahydronaphthalen-1-yl)phenyl)piperidin-4-yl)ethyl)piperazin-1-yl)-2-oxoethoxy)-1-methyl-1H-indazol-3-yl)-piperidine-2,6-dione B36;
3-(6-(4-(2-(1-(4-((1R,2S)-6-hydroxy-2-phenyl-1,2,3,4-tetrahydronaphthalen-1-yl)phenyl)piperidin-4-yl)ethyl)piperazin-1-yl)-1-methyl-1H-indazol-3-yl)piperidine-2,6-dione B37;
3-(6-(2-(4-(4-(4-((1R,2S)-6-hydroxy-2-phenyl-1,2,3,4-tetrahydronaphthalen-1-yl)phenoxy)butyl)piperazin-1-yl)ethoxy)-1-methyl-1H-indazol-3-yl)piperidine-2,6-dione B38;
3-(6-(2-(4-(4-((1R,2S)-6-hydroxy-2-phenyl-1,2,3,4-tetrahydronaphthalen-1-yl)-phenyl)piperazin-1-yl)-2-oxoethoxy)-1-methyl-1H-indazol-3-yl)piperidine-2,6-dione B39;
3-(6-(2-(4-((4-(4-((1R,2S)-6-hydroxy-2-phenyl-1,2,3,4-tetrahydronaphthalen-1-yl)phenyl)piperazin-1-yl)methyl)piperidin-1-yl)-2-oxoethoxy)-1-methyl-1H-indazol-3-yl)piperidine-2,6-dione B40;
3-(6-(4-((4-(4-((1R,2S)-6-hydroxy-2-phenyl-1,2,3,4-tetrahydronaphthalen-1-yl)-phenyl)piperidin-1-yl)methyl)piperidin-1-yl)-1-methyl-1H-indazol-3-yl)piperidine-2,6-dione;
3-(6-(4-(3-(4-(4-((1R,2S)-6-hydroxy-2-phenyl-1,2,3,4-tetrahydronaphthalen-1-yl)phenyl)piperidin-1-yl)propyl)piperidin-1-yl)-1-methyl-1H-indazol-3-yl)piperidine-2,6-dione B42;
3-(6-(4-(2-(4-(4-((1R,2S)-6-hydroxy-2-phenyl-1,2,3,4-tetrahydronaphthalen-1-yl)phenyl)piperidin-1-yl)ethyl)piperidin-1-yl)-1-methyl-1H-indazol-3-yl)piperidine-2,6-dione B43;
3-(4-(2-(4-(4-(4-((1S,2R)-6-hydroxy-2-phenyl-1,2,3,4-tetrahydronaphthalen-1-yl)phenoxy)butyl)piperazin-1-yl)-2-oxoethoxy)phenyl)piperidine-2,6-dione B44;
1-(4-(2-(4-(4-(4-((1S,2R)-6-hydroxy-2-phenyl-1,2,3,4-tetrahydronaphthalen-1-yl)phenoxy)butyl)piperazin-1-yl)-2-oxoethoxy)phenyl)dihydropyrimidine-2,4(1H,3H)-dione B45;
3-(6-(2-(4-(4-(4-((1R,2S)-6-hydroxy-2-phenyl-1,2,3,4-tetrahydronaphthalen-1-yl)phenoxy)butyl)piperazin-1-yl)-2-oxoethoxy)-1-methyl-1H-indol-3-yl)piperidine-2,6-dione B46;
3-(6-(2-(4-(4-(4-((1R,2S)-6-hydroxy-2-phenyl-1,2,3,4-tetrahydronaphthalen-1-yl)phenoxy)butyl)piperazin-1-yl)-2-oxoethoxy)benzo[b]thiophen-3-yl)piperidine-2,6-dione B47;
(2S,4R)-4-hydroxy-1-((S)-2-(2-(2-(4-(2-(4-((1R,2S)-6-hydroxy-2-phenyl-1,2,3,4-tetrahydronaphthalen-1-yl)phenoxy)ethyl)-piperazin-1-yl)ethoxy)acetamido)-3,3-dimethyl-butanoyl)-N-(4-(4-methylthiazol-5-yl)benzyl)pyrrolidine-2-carboxamide C1;
3-(4-(2-(4-(4-(4-((5-hydroxy-2-(4-hydroxyphenyl)-3-methyl-3H-indol-3-yl)-methyl)phenoxy)butyl)piperazin-1-yl)-2-oxoethoxy)-1-oxoisoindolin-2-yl)piperidine-2,6-dione D1;
3-(5-(4-(4-(4-((5-hydroxy-2-(4-hydroxyphenyl)-3-methyl-3H-indol-3-yl)-methyl)phenoxy)butyl)piperazin-1-yl)-1-oxoisoindolin-2-yl)piperidine-2,6-dione D2;
3-(5-(2-(4-(4-(4-((5-hydroxy-2-(4-hydroxyphenyl)-3-methyl-3H-indol-3-yl)-methyl)phenoxy)butyl)piperazin-1-yl)-2-oxoethoxy)-1-oxoisoindolin-2-yl)piperidine-2,6-dione D3;
3-(7-(2-(4-(4-(4-((5-hydroxy-2-(4-hydroxyphenyl)-3-methyl-3H-indol-3-yl)-methyl)phenoxy)butyl)piperazin-1-yl)-2-oxoethoxy)-1-methyl-1H-indazol-3-yl)piperidine-2,6-dione D4;
3-(6-(2-(4-(4-(4-((5-hydroxy-2-(4-hydroxyphenyl)-3-methyl-3H-indol-3-yl)-methyl)phenoxy)butyl)piperazin-1-yl)-2-oxoethoxy)-1-methyl-1H-indazol-3-yl)piperidine-2,6-dione D5;
3-(4-(2-(4-(4-(4-((5-hydroxy-2-(4-hydroxyphenyl)-3-methyl-3H-indol-3-yl)-methyl)phenoxy)butyl)piperazin-1-yl)-2-oxoethoxy)phenyl)piperidine-2,6-dione D6;
3-(3-(2-(4-(4-(4-((5-hydroxy-2-(4-hydroxyphenyl)-3-methyl-3H-indol-3-yl)-methyl)phenoxy)butyl)piperazin-1-yl)-2-oxoethoxy)phenyl)piperidine-2,6-dione D7;
1-(3-(2-(4-(4-(4-((5-hydroxy-2-(4-hydroxyphenyl)-3-methyl-3H-indol-3-yl)-methyl)phenoxy)butyl)piperazin-1-yl)-2-oxoethoxy)phenyl)dihydropyrimidine-2,4(1H,3H)-dione D8;
1-(4-(2-(4-(4-(4-((5-hydroxy-2-(4-hydroxyphenyl)-3-methyl-3H-indol-3-yl)-methyl)phenoxy)butyl)piperazin-1-yl)-2-oxoethoxy)phenyl)dihydropyrimidine-2,4(1H,3H)-dione D9;
3-(4-(2-(4-(4-(4-((5-hydroxy-2-(4-hydroxyphenyl)-3-methyl-1H-indol-1-yl)-methyl)phenoxy)butyl)piperazin-1-yl)-2-oxoethoxy)-1-oxoisoindolin-2-yl)piperidine-2,6-dione E1;
3-(5-(4-(4-(4-((5-hydroxy-2-(4-hydroxyphenyl)-3-methyl-1H-indol-1-yl)methyl)-phenoxy)butyl)piperazin-1-yl)-1-oxoisoindolin-2-yl)piperidine-2,6-dione E2;
3-(5-(2-(4-(4-(4-((5-hydroxy-2-(4-hydroxyphenyl)-3-methyl-1H-indol-1-yl)-methyl)phenoxy)butyl)piperazin-1-yl)-2-oxoethoxy)-1-oxoisoindolin-2-yl)piperidine-2,6-dione E3;
3-(5-(4-((1-(4-((5-hydroxy-2-(4-hydroxyphenyl)-3-methyl-1H-indol-1-yl)-methyl)phenyl)piperidin-4-yl)methyl)piperazin-1-yl)-1-oxoisoindolin-2-yl)piperidine-2,6-dione E4;
3-(1-((4-(((4-(4-((5-hydroxy-2-(4-hydroxyphenyl)-3-methyl-1H-indol-1-yl)-methyl)phenoxy)butyl)amino)methyl)phenoxy)methyl)-4-oxo-4H-thieno[3,4-c]pyrrol-5(6H)-yl)-piperidine-2,6-dione E5;
3-(2-(4-(4-(4-((5-hydroxy-2-(4-hydroxyphenyl)-3-methyl-1H-indol-1-yl)methyl)-phenoxy)butyl)piperazin-1-yl)-4-oxo-4,6-dihydro-5H-thieno[2,3-c]pyrrol-5-yl)piperidine-2,6-dione E6;
3-(7-(2-(4-(4-(4-((5-hydroxy-2-(4-hydroxyphenyl)-3-methyl-1H-indol-1-yl)-methyl)phenoxy)butyl)piperazin-1-yl)-2-oxoethoxy)-1-methyl-1H-indazol-3-yl)piperidine-2,6-dione E7;
3-(7-((2-(4-(4-(4-((5-hydroxy-2-(4-hydroxyphenyl)-3-methyl-1H-indol-1-yl)-methyl)phenoxy)butyl)piperazin-1-yl)-2-oxoethyl)amino)-1-methyl-1H-indazol-3-yl)piperidine-2,6-dione E8;
3-(7-(2-(4-(4-(4-((2-(4-fluorophenyl)-5-hydroxy-3-methyl-1H-indol-1-yl)-methyl)phenoxy)butyl)piperazin-1-yl)-2-oxoethoxy)-1-methyl-1H-indazol-3-yl)piperidine-2,6-dione E9;
3-(7-(2-(4-(4-(4-((5-hydroxy-3-methyl-2-(4-(trifluoromethyl)phenyl)-1H-indol-1-yl)methyl)phenoxy)butyl)piperazin-1-yl)-2-oxoethoxy)-1-methyl-1H-indazol-3-yl)piperidine-2,6-dione E10;
3-(7-(2-(4-(4-(4-((2-(4-(difluoromethyl)phenyl)-5-hydroxy-3-methyl-1H-indol-1-yl)methyl)phenoxy)butyl)piperazin-1-yl)-2-oxoethoxy)-1-methyl-1H-indazol-3-yl)piperidine-2,6-dione E11;
3-(7-(2-(4-(4-((5-hydroxy-2-(4-hydroxyphenyl)-3-methyl-1H-indol-1-yl)methyl)-phenyl)piperazin-1-yl)-2-oxoethoxy)-1-methyl-1H-indazol-3-yl)piperidine-2,6-dione E12;
3-(7-(2-(4-(4-(4-((5-hydroxy-2-(4-hydroxyphenyl)-3-methyl-1H-indol-1-yl)-methyl)phenoxy)butyl)piperazin-1-yl)ethoxy)-1-methyl-1H-indazol-3-yl)piperidine-2,6-dione E13;
3-(6-(2-(4-(4-(4-((5-hydroxy-2-(4-hydroxyphenyl)-3-methyl-1H-indol-1-yl)-methyl)phenoxy)butyl)piperazin-1-yl)-2-oxoethoxy)-1-methyl-1H-indazol-3-yl)piperidine-2,6-dione E14;
3-(6-(2-(4-((1-(4-((5-hydroxy-2-(4-hydroxyphenyl)-3-methyl-1H-indol-1-yl)-methyl)phenyl)piperidin-4-yl)methyl)piperazin-1-yl)-2-oxoethoxy)-1-methyl-1H-indazol-3-yl)-piperidine-2,6-dione E15;
1-(6-(2-(4-(4-(4-((5-hydroxy-2-(4-hydroxyphenyl)-3-methyl-1H-indol-1-yl)-methyl)phenoxy)butyl)piperazin-1-yl)-2-oxoethoxy)-1-methyl-1H-indazol-3-yl)dihydro-pyrimidine-2,4(1H,3H)-dione E16;
3-(6-(4-((1-(4-((5-hydroxy-2-(4-hydroxyphenyl)-3-methyl-1H-indol-1-yl)-methyl)phenyl)piperidin-4-yl)methyl)piperazin-1-yl)-1-methyl-1H-indazol-3-yl)piperidine-2,6-dione E17;
3-(6-(4-(2-(1-(4-((5-hydroxy-2-(4-hydroxyphenyl)-3-methyl-1H-indol-1-yl)-methyl)phenyl)piperidin-4-yl)ethyl)piperazin-1-yl)-1-methyl-1H-indazol-3-yl)piperidine-2,6-dione E18;
3-(6-(2-(4-(3-(1-(4-((5-hydroxy-2-(4-hydroxyphenyl)-3-methyl-1H-indol-1-yl)-methyl)phenyl)piperidin-4-yl)propyl)piperazin-1-yl)-2-oxoethoxy)-1-methyl-1H-indazol-3-yl)-piperidine-2,6-dione E19;
3-(6-(4-(3-(1-(4-((5-hydroxy-2-(4-hydroxyphenyl)-3-methyl-1H-indol-1-yl)-methyl)phenyl)piperidin-4-yl)propyl)piperazin-1-yl)-1-methyl-1H-indazol-3-yl)piperidine-2,6-dione E20;
3-(6-(2-(4-(2-(1-(4-((5-hydroxy-2-(4-hydroxyphenyl)-3-methyl-1H-indol-1-yl)-methyl)phenyl)piperidin-4-yl)ethyl)piperazin-1-yl)-2-oxoethoxy)-1-methyl-1H-indazol-3-yl)-piperidine-2,6-dione E21;
3-(6-(2-(4-(4-(4-((5-hydroxy-3-methyl-2-(4-(trifluoromethyl)phenyl)-1H-indol-1-yl)methyl)phenoxy)butyl)piperazin-1-yl)-2-oxoethoxy)-1-methyl-1H-indazol-3-yl)piperidine-2,6-dione E22;
1-(7-(2-(4-(4-(4-((5-hydroxy-2-(4-hydroxyphenyl)-3-methyl-1H-indol-1-yl)-methyl)phenoxy)butyl)piperazin-1-yl)-2-oxoethoxy)-1-methyl-1H-indazol-3-yl)dihydro-pyrimidine-2,4(1H,3H)-dione D23;
3-(4-(2-(4-(4-(4-((5-hydroxy-2-(4-hydroxyphenyl)-3-methyl-1H-indol-1-yl)-methyl)phenoxy)butyl)piperazin-1-yl)-2-oxoethoxy)phenyl)piperidine-2,6-dione E24;
3-(3-(2-(4-(4-(4-((5-hydroxy-2-(4-hydroxyphenyl)-3-methyl-1H-indol-1-yl)-methyl)phenoxy)butyl)piperazin-1-yl)-2-oxoethoxy)phenyl)piperidine-2,6-dione E25;
1-(3-(2-(4-(4-(4-((5-hydroxy-2-(4-hydroxyphenyl)-3-methyl-1H-indol-1-yl)-methyl)phenoxy)butyl)piperazin-1-yl)-2-oxoethoxy)phenyl)dihydropyrimidine-2,4(1H,3H)-dione E26; or
1-(4-(2-(4-(4-(4-((5-hydroxy-2-(4-hydroxyphenyl)-3-methyl-1H-indol-1-yl)-methyl)phenoxy)butyl)piperazin-1-yl)-2-oxoethoxy)phenyl)dihydropyrimidine-2,4(1H,3H)-dione E27;
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

197. A pharmaceutical composition comprising the compound of any one of claims 1 to 196, or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof, and a pharmaceutically acceptable excipient.

198. The pharmaceutical composition of claim 197, wherein the composition is in single dosage form.

199. The pharmaceutical composition of claim 197 or 198, wherein the composition is in an oral, parenteral, or intravenous dosage form.

200. The pharmaceutical composition of claim 199, wherein the composition is formulated in an oral dosage form.

201. The pharmaceutical composition of claim 200, wherein the oral dosage form is a tablet or capsule.

202. A method of treating, preventing, or ameliorating one or more symptoms of a disorder, disease, or condition mediated by an estrogen receptor in a subject, comprising administering to the subject in need thereof a therapeutically effective amount of a compound of any one of claims 1 to 196 or a pharmaceutical composition of any one of claims 197 to 201.

203. The method of claim 202, wherein the disorder, disease, or condition mediated by an estrogen receptor is a proliferative disease.

204. A method of treating, preventing, or ameliorating one or more symptoms of a proliferative disease in a subject, comprising administering to the subject in need thereof a therapeutically effective amount of a compound of any one of claims 1 to 196 or a pharmaceutical composition of any one of claims 197 to 201.

205. The method of claim 203 or 204, wherein the proliferative disease is cancer.

206. The method of any one of claims 203 to 205, wherein the proliferative disease is breast cancer.

207. The method of any one of claims 203 to 206, wherein the proliferative disease is ERα+ breast cancer.

208. The method of any one of claims 203 to 207, wherein the cancer is relapsed or refractory.

209. The method of any one of claims 203 to 208, wherein the cancer is metastatic.

210. The method of any one of claims 203 to 209, wherein the cancer is drug-resistant.

211. The method of any one of claims 202 to 210, wherein the subject is a human.

212. A method of inhibiting the growth of a cell, comprising contacting the cell with an effective amount of a compound of a compound of any one of claims 1 to 178 or a pharmaceutical composition of any one of claims 179 to 183.

213. The method of claim 194, wherein the cell is a cancerous cell.

214. A method of inducing degradation of an estrogen receptor, comprising contacting the estrogen receptor with an effective amount of a compound of a compound of any one of claims 1 to 196 or a pharmaceutical composition of any one of claims 197 to 201.

215. The method of claim 201, wherein the estrogen receptor is an estrogen receptor-α.
